(19) United States
(12) Patent Application Publication
(10) Pub. No.: US 2026/0144881 A1
(43) Pub. Date: May 28, 2026

(54) HPK1 TARGETING COMPOUNDS AND USES THEREOF

(71) Applicant: Arvinas Operations, Inc., New Haven, CT (US)

(72) Inventors: Albert M. DeBerardinis, West Hartford, CT (US); Jesus Raul Medina, Gilbertsville, PA (US); Alexey Ishchenko, Walpole, MA (US); Hanqing Dong, Madison, CT (US); Song Yang, Springfield, NJ (US); William Leslie Corwin, Canton, CT (US); Arman Fathizadeh, Amarillo, TX (US)

(21) Appl. No.: 19/361,211

(22) Filed: Oct. 17, 2025

Related U.S. Application Data
(63) Continuation of application No. PCT/US2025/048229, filed on Sep. 26, 2025.
(60) Provisional application No. 63/699,528, filed on Sep. 26, 2024.

(51) Int. Cl. A61K 47/55

(52) U.S. Cl. CPC A61K 47/55

(57) ABSTRACT

Provided are compounds of the Formula (I):

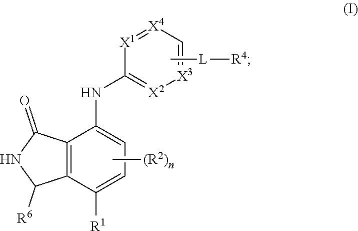

or pharmaceutically acceptable salts thereof, which are useful for the degradation of HPK1 and in the treatment of a variety of HPK1 mediated conditions or diseases, such as cancer.

### RELATED APPLICATIONS

This application is a continuation application of International Application No. PCT/US2025/048229, filed on Sep. 26, 2026, which in turn claims priority to U.S. Provisional Application No. 63/699,528, filed on Sep. 26, 2024. The entire contents of each of the foregoing applications are expressly incorporated herein by reference.

### FIELD OF THE INVENTION

The present disclosure relates to degraders of HPK1, and pharmaceutically acceptable salts thereof, compositions of these compounds, processes for their preparation, and their use in the treatment of diseases.

### BACKGROUND

Hematopoietic Progenitor Kinase 1 (HPK1) also known as MAP4K1, is a serine-threonine kinase from the germinal center family of kinases, which regulates the function of a number of immune cells, including T cells, B cells, and dendritic cells. HPK1 functions as a negative immune regulator of activation signals generated by the T cell antigen receptor (TCR) and B-cell signaling. In T-cells, following T-cell receptor activation, HPK1 is recruited to the plasma membrane where it phosphorylates the adapter protein SH2 domain-containing leukocyte protein, down-regulating signaling events required for T cell activation and proliferation, and suppressing the innate immune response. HPK1 inhibitors block inhibitory signals that would reduce the ability of T cells to clear the tumor and has been recognized as a novel antitumor immunotherapy target. HPK1 inhibition and knockdown shows enhanced T-cell signaling, cytokine production, and in vivo tumor growth inhibition. The need for new HPK1 compounds which target HPK1 are therefore needed to treat cancer and other conditions responsive to the inhibition or degradation of HPK1.

### SUMMARY

Provided herein are compounds that modulate hematopoietic progenitor kinase (HPK1). In certain embodiments, the compounds described herein are inhibitors of HPK1. In certain embodiments, the compounds described herein are degraders of HPK1. In embodiments, the compounds are of Formula (I):

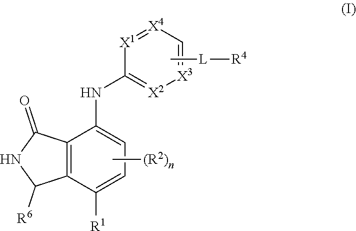

and pharmaceutically acceptable salt and compositions thereof, wherein X¹, X², X³, X⁴, L, R¹, R², R⁴, R⁶, and n are as defined herein.

Also described herein are pharmaceutical compositions comprising one or more compounds described herein and a pharmaceutically acceptable excipient.

In certain embodiments, described herein are methods treating cancer, comprising administering a therapeutically effective amount of one or more compounds described herein, or a pharmaceutical composition thereof, to a subject in need thereof.

In certain embodiments, described herein are methods of inhibiting the activity of hematopoietic progenitor kinase (HPK1), comprising administering a therapeutically effective amount one or more compounds described herein, or a pharmaceutical composition thereof, to a subject.

In certain embodiments, the compounds of the present disclosure have improved pharmacokinetic properties, such as improved oral bioavailability. In certain embodiments, the compounds of the present disclosure have improved HPK1 degradation activity.

### BRIEF DESCRIPTION OF THE DRAWINGS

FIG. 1 shows the percentage of responders in the in vivo efficacy test of elected compounds in a CT-26 mouse model.

### DETAILED DESCRIPTION

### 1. General Description of Compounds

In embodiments, provided herein is a compound of Formula (I):

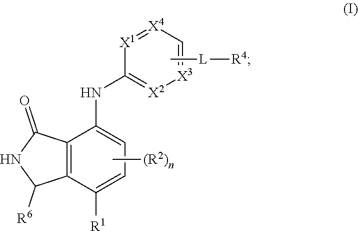

or a pharmaceutically acceptable salt thereof, wherein:
R¹ is C₃₋₆cycloalkyl, phenyl, naphthyl, 4- to 12-membered heterocyclyl or 5- to 10-membered heteroaryl, each of which is optionally substituted by one or more R⁵;
n is 0, 1, or 2;
each R² is independently halo, C₁₋₄alkyl, C₁₋₄haloalkyl, C₃₋₆cyloalkyl, —OH, C₁₋₄alkoxy, C₁₋₄haloalkoxy, —N(C₁₋₄alkyl)₂, —NH₂, —NH(C₁₋₄alkyl), or cyano;
X¹, X², X³ and X⁴ are each independently N or —CR³, provided that at least two of X¹, X², X³ and X⁴ are —CR³;
each R³ is independently hydrogen, halo, —OR^{O3a}, —NR^{N3a}R^{N3b}, cyano, C₁₋₄alkyl, C₁₋₄haloalkyl, C₂alkynyl, —C(O)R^{3a}, —SO₂R^{3a}, —NR^{N3a}SO₂R^{3a}, C₃₋₆cycloalkyl, or 5- to 6-membered monocyclic heterocyclyl, wherein the C₁₋₄alkyl is optionally substituted by one or more R^{3a};
each R^{3a} is independently —OR^{O3a}, —NR^{N3a}R^{N3b}, or 5- to 6-membered monocyclic heterocyclyl;
R^{O3a}, R^{N3a}, and R^{N3b} are each independently hydrogen, C₁₋₄alkyl, or C₁₋₄haloalkyl;
R⁴ is:

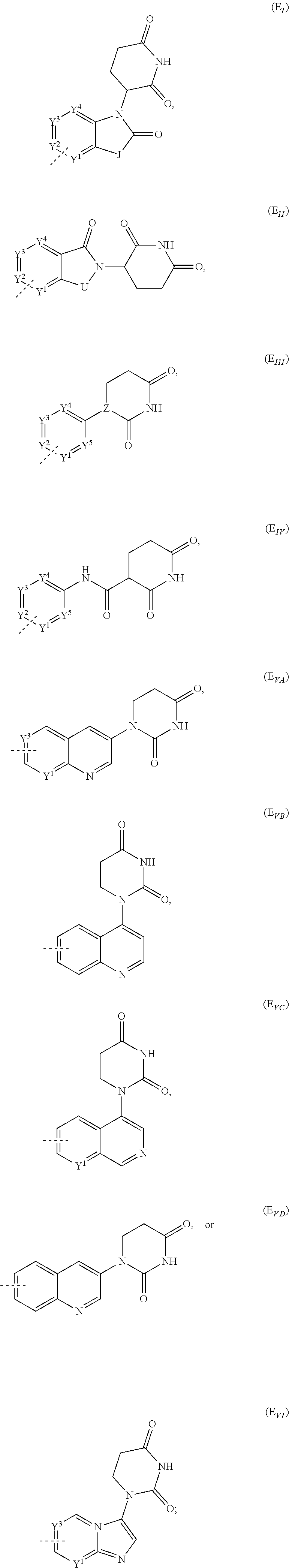

J is NR^{R1} or C(R^{E2})₂;
U is C═O or C(R^{E2})₂
R^{E1} is C₁₋₄alkyl, C₁₋₄haloalkyl, or C₃₋₆cycloalkyl;
each R^{E2} is independently hydrogen, C₁₋₄alkyl, or C₁₋₄haloalkyl;
Z is N or CH;
each of Y¹, Y², Y³, Y⁴, and Y⁵ is independently CR^{Y} or N;
each R^{Y} is independently hydrogen, halo, C₁₋₃alkyl, C₁₋₃haloalkyl, C₁₋₃alkoxy, or C₁₋₃haloalkoxy
each R⁵ is independently halo, C₁₋₄alkyl, C₁₋₄haloalkyl, —OH, C₁₋₄alkoxy, C₁₋₄haloalkoxy, —N(C₁₋₄alkyl)₂, —NH₂, —NH(C₁₋₄alkyl), or cyano;
R⁶ is hydrogen, halo, C₁₋₄alkyl, or C₁₋₄haloalkyl; and
L is:

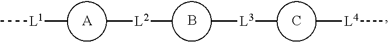

wherein:
Ring A, Ring B, and Ring C are each absent or a 4- to 10-membered carbocyclyl, 4- to 12-membered heterocyclyl, phenyl, or 5- to 10-membered heteroaryl, each of which are optionally substituted by one or more R^{L};
each R^{L} is independently halo, C₁₋₄alkyl, or C₁₋₄haloalkyl;
L¹, L², L³, and L⁴ are each absent or C₁₋₄alkylene, wherein the C₁₋₄alkylene is optionally substituted by one or more R^{L1}, and wherein one or more C in the C₁₋₄alkylene is optionally replaced by O; and
each R^{L1} is independently halo, or two R^{L1} groups, together with the carbon to which they are attached, form oxo or C₃₋₆cycloalkyl.

In embodiments, provided herein is a compound of Formula (I):

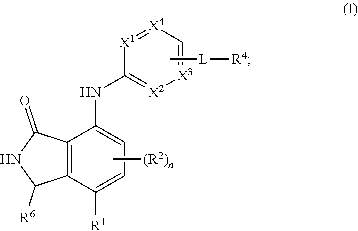

or a pharmaceutically acceptable salt thereof, wherein:
R¹ is C₃₋₆cycloalkyl, phenyl, 4- to 12-membered heterocyclyl or 5- to 10-membered heteroaryl, each of which is optionally substituted by one or more R⁵;
n is 0, 1, or 2;
each R² is independently halo, C₁₋₄alkyl, C₁₋₄haloalkyl, —OH, C₁₋₄alkoxy, C₁₋₄haloalkoxy, —N(C₁₋₄alkyl)₂, —NH₂, —NH(C₁₋₄alkyl), or cyano;
X¹, X², X³ and X⁴ are each independently N or —CR³, provided that at least two of X¹, X², X³ and X⁴ are —CR³;
each R³ is independently hydrogen, halo, —OR^{O3a}, —NR^{N3a}R^{N3b}, cyano, C₁₋₄alkyl, C₁-4haloalkyl, C₂alkynyl, —C(O)R^{3a}, —SO₂R^{3a}, —NR^{N3a}SO₂R^{3a}, C₃₋₆cycloalkyl, or 5- to 6-membered monocyclic heterocyclyl, wherein the C₁₋₄alkyl is optionally substituted by one or more R^{3a};
each R^{3a} is independently —OR^{O3a}, —NR^{N3a}R^{N3b}, or 5- to 6-membered monocyclic heterocyclyl;
R^{O3a}, R^{N3a}, and R^{N3b} are each independently hydrogen, C₁₋₄alkyl, or C₁₋₄haloalkyl;
R⁴ is:

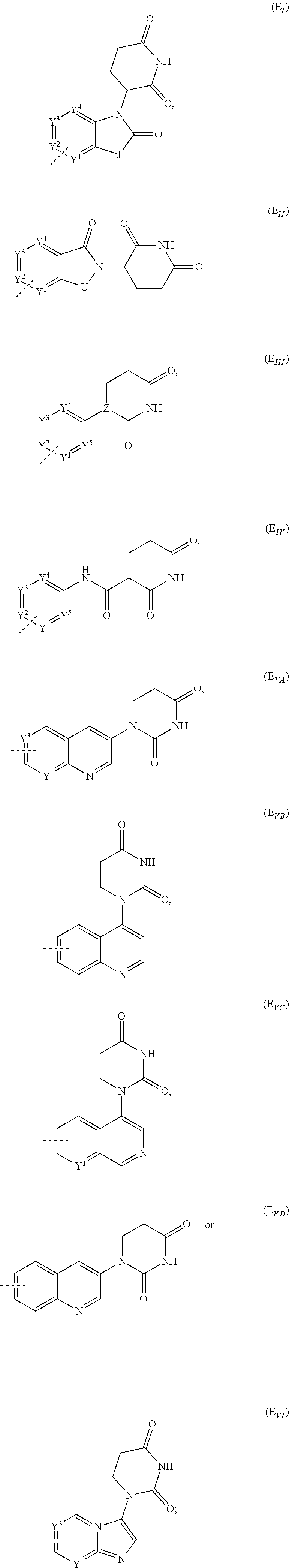

J is NR^{R1} or C(R^{E2})₂;
U is C═O or C(R^{E2})₂
R^{E1} IS C₁₋₄alkyl or C₃₋₆cycloalkyl;
each R^{E2} is independently hydrogen, C₁₋₄alkyl, or C₁₋₄haloalkyl;
Z is N or CH;
each of Y¹, Y², Y³, Y⁴, and Y⁵ is independently CR^{Y} or N;
each R is independently hydrogen, halo, C₁₋₃alkyl, C₁₋₃haloalkyl, C₁₋₃alkoxy, or C₁₋₃haloalkoxy
each R⁵ is independently halo, C₁₋₄alkyl, C₁₋₄haloalkyl, —OH, C₁₋₄alkoxy, C₁₋₄haloalkoxy, —N(C₁₋₄alkyl)₂, —NH₂, —NH(C₁₋₄alkyl), or cyano;
R⁶ is hydrogen, halo, C₁₋₄alkyl, or C₁₋₄haloalkyl; and
L is:
   wherein:

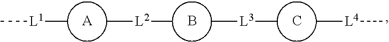

Ring A, Ring B, and Ring C are each absent or a 4- to 10-membered carbocyclyl, 4- to 12-membered heterocyclyl, phenyl, or 5- to 10-membered heteroaryl, each of which are optionally substituted by one or more R^{L};
each R^{L} is independently halo, C₁₋₄alkyl, or C₁₋₄haloalkyl;
L¹, L², L³, and L⁴ are each absent or C₁₋₄alkylene, wherein the C₁₋₄alkylene is optionally substituted by one or more R^{L1}, and wherein one or more C in the C₁₋₄alkylene is optionally replaced by O; and
each R^{L1} is independently halo, or two R^{L1} groups, together with the carbon to which they are attached, form oxo or C₃₋₆cycloalkyl.

### 2. Definitions

In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element, unless otherwise indicated.

All transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of shall be closed or semi-closed transitional phrases, respectively, as set forth in the United States Patent Office Manual of Patent Examining Procedures, Section 2111.03.

When a range of carbon atoms is used herein, for example, C₁-C₆ or C₁₋₆, all ranges, as well as individual numbers of carbon atoms are encompassed. For example, “C₁₋₃” includes C₁₋₃, C₁₋₂, C₂₋₃, C₁, C₂, and C₃.

The terms “halo” and “halogen” refer to an atom selected from fluorine (fluoro, F), chlorine (chloro, Cl), bromine (bromo, Br), and iodine (iodo, I).

The term “alkyl” when used alone or as part of a larger moiety, such as “haloalkyl”, “hydroxyalkyl” and the like, means saturated straight-chain or branched monovalent hydrocarbon radical having, unless otherwise specified, from 1 to 20 carbon atoms such as C₁₋₁₀, C₁₋₆, or C₁₋₄. A C₁₋₆ alkyl includes e.g., methyl, ethyl, propyl (e.g., n-propyl, isopropyl), butyl (e.g., n-butyl, tert-butyl, sec-butyl, iso-butyl), pentyl (e.g., n-pentyl, 3-pentanyl, amyl, neopentyl, 3-methyl-2-butanyl, tertiary amyl), and hexyl (e.g., n-hexyl). It will be understood that when specified, optional substituents on an alkyl group may be present on any substitutable position.

The term “alkylene” refers to a bivalent alkyl group.

The term “haloalkyl” includes mono, poly, and perhaloalkyl groups where the halogens are independently selected from fluorine, chlorine, bromine, and iodine.

The term “hydroxyalkyl” includes mono, poly, and perhydroxy alkyl groups where one or more hydrogen atoms are replaced by OH.

The term “alkoxy” refers to an alkyl radical attached through an oxygen linking atom, represented by —Oalkyl. Non-limiting examples include methoxy, ethoxy, propoxy, 2-propoxy, butoxy, tert-butoxy, pentyloxy, and hexyloxy. It will be understood that when specified, optional substituents on an alkoxy group may be present on any substitutable position

The term “haloalkoxy” includes mono, poly, and perhaloalkoxy groups where the halogens are independently selected from fluorine, chlorine, bromine, and iodine.

The term “heteroaryl” refers to, unless otherwise specified, a 5-16 membered aromatic radical containing 1-4 heteroatoms selected from N, O, and S. In some instances, nitrogen atoms in a heteroaryl may be quarternized. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, benzooxazolyl, benzooxodiazolyl, indazolyl, benzimidazolyl, benzthiazolyl, benzothiopheneyl, quinolinyl, quinazolinyl, quinoxalinyl, pyrrolopyridinyl, pyrrolopyrimidinyl, pyrrolopyridinyl, thienopyridinyl, thienopyrimidinyl, indolizinyl, purinyl, cinnolinyl, naphthyridinyl, and pteridinyl. It will be understood that when specified, optional substituents on a heteroaryl group may be present on any substitutable position and, include, e.g., the position at which the heteroaryl is attached (where valency permits).

The term “heterocyclyl” means, unless otherwise specified, a 4- to 12-membered saturated or partially unsaturated heterocyclic ring containing 1 to 4 heteroatoms independently selected from N, O, and S. A heterocyclyl ring can be attached to its pendant group at any heteroatom or carbon atom that results in a stable structure. A heterocyclyl group may be mono- or bicyclic (e.g., a bridged, fused, or spiro bicyclic ring). Examples of monocyclic saturated or partially unsaturated heterocyclic radicals include, without limitation, azetidinyl, tetrahydrofuranyl, tetrahydrothienyl, terahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, tetrahydropyrimidinyl, dihydrooxadizolyl, and dihydroisoxazolyl. Bi-cyclic heterocyclyl groups include, e.g., unsaturated heterocyclic radicals fused to another unsaturated heterocyclic radical, cycloalkyl, aryl, or heteroaryl ring, such as for example, benzodioxolyl, dihydrobenzodioxinyl, dihydrobenzofuranyl, dihydrobenzothiophenyl, 5-oxa-2,6-diazaspiro[3.4]oct-6-enyl, 6-thia-2,7-diazaspiro[3.4]octanyl, 2,6-diazaspiro[3.3]heptanyl, spiro[indoline-3,3′-pyrrolidine]-yl, thiochromanyl, 7-azaspiro[3.5]nonanyl, 2,7-diazaspiro[3.5]nonanyl, 3-azaspiro[5.5]undecayl, 2-azaspiro[3.3]heptanyl, 3,9-diazaspiro[5.5]undecanyl, 6-azaspiro[3.4]octanyl and the like. It will be understood that when specified, optional substituents on a heterocyclyl group may be present on any substitutable position and, include, e.g., the position at which the heterocyclyl group is attached.

The term “spiro” refers to two rings that shares one ring atom (e.g., carbon).

The term “fused” refers to two rings that share two adjacent ring atoms with one another.

The term “bridged” refers to two rings that share three adjacent ring atoms with one another.

The term “cycloalkyl” refers to a saturated cyclic aliphatic monocyclic or bicyclic ring system, as described herein, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl or cycloaliphatic group may be present on any substitutable position and, include, e.g., the position at which the cycloalkyl group is attached.

The term “optionally substituted” means that one or more hydrogens of the designated moiety may be replaced with a suitable substituent. Unless otherwise indicated, an “optionally substituted” group may have a suitable substituent at each substitutable position of the group as valency permits. Optional substituents include, but are not limited to, one or more groups selected from cyano (—CN), halo, imino (═NH), nitro (—NO₂), oxo (═O), —C(O)Rⁱ, —C(O)ORⁱ, —C(O)NRⁱⁱRⁱⁱⁱ, —C(O)SRⁱ, —C(NRⁱ)NRⁱⁱRⁱⁱⁱ, —C(S)Rⁱ, —C(S)ORⁱ, —C(S)NRⁱⁱRⁱⁱⁱ, —ORⁱ, —OC(O)Rⁱ, —OC(O)ORⁱ, —OC(O)NRⁱⁱRⁱⁱⁱ, —OC(O)SRⁱ, —OC(NRⁱ)NRⁱⁱRⁱⁱⁱ, —OC(S)Rⁱ, —OC(S)ORⁱ, —OC(S)NRⁱⁱRⁱⁱⁱ, —OP(O)(ORⁱⁱ)ORⁱⁱⁱ, —OS(O)Rⁱ, —OS(O)₂Rⁱ, —OS(O)NRⁱⁱRⁱⁱⁱ, —OS(O)₂NRⁱⁱRⁱⁱⁱ, —NRⁱⁱRⁱⁱⁱ, —NRⁱC(O)R^{iv}, —NRⁱC(O)OR^{iv}, —NRⁱC(O)NRⁱⁱRⁱⁱⁱ, —NR^{a}C(O)SR^{iv}, —NRⁱC(NR^{iv})NRⁱⁱRⁱⁱⁱ, —NRⁱC(S)R^{iv}, —NRⁱC(S)OR^{iv}, —NRⁱC(S)NRⁱⁱRⁱⁱⁱ, —NRⁱS(O)R^{iv}, —NRⁱS(O)₂R^{iv}, —NRⁱS(O)NRⁱⁱRⁱⁱⁱ, —NRⁱS(O)₂NRⁱⁱR^{iv}, —SR, —S(O)Rⁱ, —S(O)₂Rⁱ, —S(O)NRⁱⁱR^{iv}, —S(O)₂NRⁱⁱRⁱⁱⁱ, alkyl, heteroalkyl, alkenyl, alkynyl, cycloalkyl, aryl, heteroaryl, and heterocyclyl, wherein said alkyl, heteroalkyl, alkenyl, alkynyl, cycloalkyl, aryl, heteroaryl, and heterocyclyl are each further optionally substituted with one or more, in one embodiment, one, two, three, or four, substituents Q^{a}, wherein each Rⁱ, Rⁱⁱ, Rⁱⁱⁱ, and R^{iv} is independently alkyl, heteroalkyl, alkenyl, alkynyl, cycloalkyl, aryl, heteroaryl, or heterocyclyl, each of which is optionally substituted with one or more, in one embodiment, one, two, three, or four, substituents Q^{a} or Rⁱⁱ and Rⁱⁱ together with the N atom to which they are attached form heterocyclyl optionally substituted with one or more, in one embodiment, one, two, three, or four, substituents Q^{a}, wherein each Q^{a} is independently selected from cyano, halo, imino, nitro, oxo, alkyl, heteroalkyl, alkenyl, alkynyl, cycloalkyl, C₆₋₁₄ aryl, heteroaryl, heterocyclyl, —C(O)R^{v}, —C(O)OR^{v}, —C(O)NR^{vi}R^{vii}, —C(O)SR^{v}, —C(NR^{v})NR^{vi}R^{vii}, —C(S)R^{v}, C(S)OR^{v}, —C(S)NR^{vi}R^{vii}, —OR^{v}, —OC(O)R^{v}, OC(O)OR^{v}, —OC(O)NR^{vi}R^{vii}, —OC(O)SR^{v}, —OC(NR^{v})NR^{vi}R^{vii}, —OC(S)R^{v}, —OC(S)OR^{v}, —OC(S)NR^{vi}R^{vii}, —OP(O)(OR^{v})OR^{vi}, —OS(O)R^{v}, —OS(O)₂R^{v}, —OS(O)NR^{vi}R^{vii}, —OS(O)₂NR^{v}R^{vii}, —NR^{vi}R^{vii}, —NR^{v}C(O)R^{viii}, —NR^{e}C(O)OR^{vi}, —NR^{v}C(O)NR^{vi}R^{vii}, —NR^{v}C(O)SR^{vi}, —NR^{v}C(NR^{viii})NR^{vi}R^{vii}, —NR^{v}C(S)R^{viii}, —NR^{v}C(S)OR^{vi}, —NR^{v}C(S)NR^{vi}R^{vii}, —NR^{vi}S(O)R^{viii}, —NR^{v}S(O)₂R^{viii}, —NR^{v}S(O)NR^{vi}R^{vii}, —NR^{v}S(O)₂NR^{vi}R^{vii}, —SR^{v}, —S(O)R^{v}, —S(O)₂R^{v}, —S(O)NR^{vi}R^{vii}, and —S(O)₂NR^{vi}R^{vii}; wherein each R^{v}, R^{vi}, R^{vii}, and R^{viii} is independently alkyl, alkenyl, alkynyl, cycloalkyl, aryl, aralkyl, heteroaryl, or heterocyclyl; or (iii) R^{vi} and R^{viii} together with the N atom to which they are attached form heterocyclyl.

In certain aspects, where specified, one or more hydrogen atoms on a disclosed compound may be replaced with deuterium. Such deuterated compounds may have one or more improved pharmacokinetic or pharmacodynamic properties (e.g., longer half-life) compared to the equivalent “un-deuterated” compound.

One or more of the compounds described herein may exist in various tautomeric forms and are part of the present disclosure. The terms “tautomers” or “tautomeric” refer to two or more interconvertible compounds/substituents resulting from at least one formal migration of a hydrogen atom and at least one change in valency. All such isomeric forms of such compounds are expressly included. Thus, when a compound herein is represented by a structural formula or designated by a chemical name herein, all tautomeric forms which may exist for the compound are encompassed by the structural formula.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. A “geometric isomer” refers to isomers that differ in the orientation of substituent group in relationship to a carbon-carbon double bond, a cycloalkyl ring, or a bridged bicyclic system. Atoms (other than H) on each side of a carbon-carbon double bond may be in an E (substituents are on opposite sides of the carbon-carbon double bond) or Z (substituents are oriented on the same side) configuration. “Cis” refers to substituents oriented on the same side of the ring, whereas “trans” refers to substituents oriented on opposite sides of the ring.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a geometric isomer is depicted by name or structure, the enrichment of the indicated isomer relative to the opposite isomer is greater than 50%, 60%, 70%, 80%, 90%, 99% or 99.9%. “Enrichment of the indicated isomer relative to the opposite isomer” is a mole percent and is determined by dividing the number of compounds with the indicated geometrical configuration by the total number of all of the compounds with the same or opposite geometrical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The terms “effective amount” and “therapeutically effective amount” of a compound described herein refers to an amount sufficient to induce a particular response in the subject, e.g., to provide a therapeutic benefit in the treatment of a condition described herein. The therapeutically effective amount of a compound of the disclosure or a pharmaceutically acceptable salt thereof may vary depending upon the intended application (in vitro or in vivo), the subject and disease condition being treated, e.g., the weight and age of the subject, the severity of the disease condition, the manner of administration and the like. Therapeutically effective amounts or doses of the compounds and pharmaceutically acceptable salts of the compounds described herein may be ascertained by routine methods such as modeling, dose escalation studies or clinical trials, and by taking into consideration factors such as, e.g., the mode or route of administration or drug delivery, the pharmacokinetics of the compound or salt, the severity and course of the disease or disorder, the subject's previous or ongoing therapy, the subject's health status and response to drugs, and the judgment of the treating physician. An illustrative example of a dose for a subject is in the range of from about 0.001 mg to about 1000 mg of compound (per day, in single or divided dosage units (e.g., BID, TID, QID).

The terms “condition”, “disease”, and “disorder” are used interchangeably herein and mean an abnormal condition that negatively affects the structure or function of all or part of a subject, and that is not immediately due to any external injury. In some embodiments, a disease is a medical condition, illness or sickness that is associated with one or more specific signs and symptoms.

The terms “administer,” “administering,” and “administration” refer to providing, implanting, absorbing, ingesting, injecting, inhaling, or otherwise introducing a compound described herein, or a pharmaceutically acceptable salt or pharmaceutical composition thereof, to, in or on a subject.

Unless otherwise indicated, the term “HPK1” includes both wild-type HPK1 and mutant forms therefore.

A “HPK1 related condition” refers to a condition that is responsive to the modulation of HPK1 such as e.g., conditions which are modulated by degrading the HPK1 protein. HPK1 related conditions may arise from protein expression, overexpression, mutation, misfolding, or dysregulation (e.g., the amount of protein expressed in a patient is elevated).

### 3. Compounds and Compositions

In embodiments, for the compound of Formula (I), or a pharmaceutically acceptable salt thereof, X⁴ and X³ are each CR³.

In embodiments, for the compound of Formula (I), or a pharmaceutically acceptable salt thereof, X¹ is N.

In embodiments, for the compound of Formula (I), or a pharmaceutically acceptable salt thereof, X² is CR³.

In embodiments, for the compound of Formula (I), or a pharmaceutically acceptable salt thereof, R¹ is naphthyl.

In embodiments, for the compound of Formula (I), or a pharmaceutically acceptable salt thereof, R¹ is a 9- to 12-membered bicyclic heterocyclyl 6-membered monocyclic heteroaryl, or 8- to 10-membered bicyclic heteroaryl, each of which is optionally substituted by one or two R⁵.

In embodiments, for the compound of Formula (I), or a pharmaceutically acceptable salt thereof, R¹ is a 9- to 12-membered bicyclic heterocyclyl or 8- to 10-membered bicyclic heteroaryl, each of which is optionally substituted by one or two R⁵.

In embodiments, for the compound of Formula (I), or a pharmaceutically acceptable salt thereof, R¹ is a 9-membered bicyclic heterocyclyl or 9-membered bicyclic heteroaryl, each of which is optionally substituted by one or two R⁵.

In embodiments, for the compound of Formula (I), or a pharmaceutically acceptable salt thereof, R¹ is 1H-pyrrolo[2,3-b]pyridinyl, 5,6,7,8-tetrahydroimidazo[1,2-a]pyridinyl, imidazo[1,2-a]pyrazinyl, imidazo[1,2-a]pyridinyl, or [1,2,4]triazolo[4,3-a]pyridinyl, each of which is optionally substituted by one or two R⁵.

In embodiments, for the compound of Formula (I), or a pharmaceutically acceptable salt thereof, R¹ is pyrimidinyl, naphthyl, quinolinyl, naphthyridinyl, benzimidazolyl, imidazo[1,2-b]pyridazinyl, benzisothiazolyl, 3H-imidazo[4,5-c]pyridinyl, 1H-pyrazolo[4,3-b]pyridinyl, 1H-pyrrolo[2,3-b]pyridinyl, 5,6,7,8-tetrahydroimidazo[1,2-a]pyridinyl, imidazo[1,2-a]pyrazinyl, imidazo[1,2-a]pyridinyl, or [1,2,4]triazolo[4,3-a]pyridinyl, each of which is optionally substituted by one or two R⁵.

In embodiments, for the compound of Formula (I), or a pharmaceutically acceptable salt thereof, R¹ is:

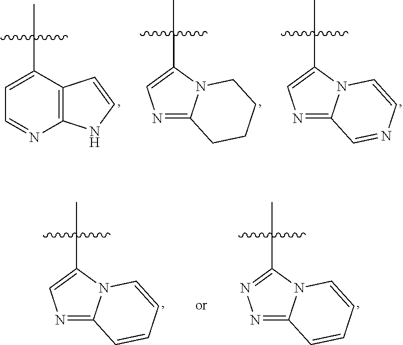

each of which is optionally substituted by one or two R⁵.

In embodiments, for the compound of Formula (I), or a pharmaceutically acceptable salt thereof, R¹ is:

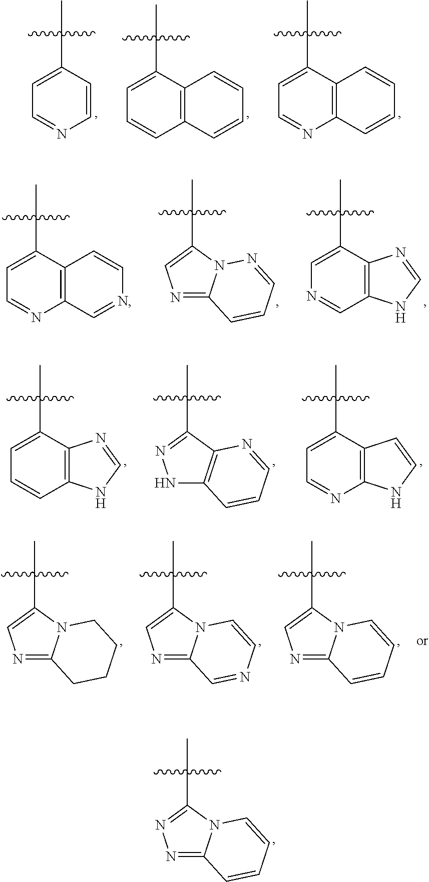

each of which is optionally substituted by one or two R⁵.

In embodiments, for the compound of Formula (I), or a pharmaceutically acceptable salt thereof, R¹ is:

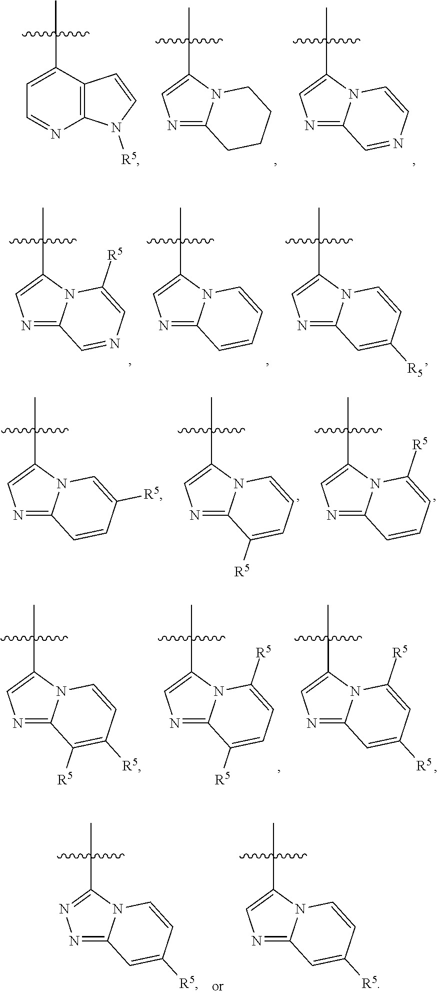

In embodiments, for the compound of Formula (I), or a pharmaceutically acceptable salt thereof, R¹ is:

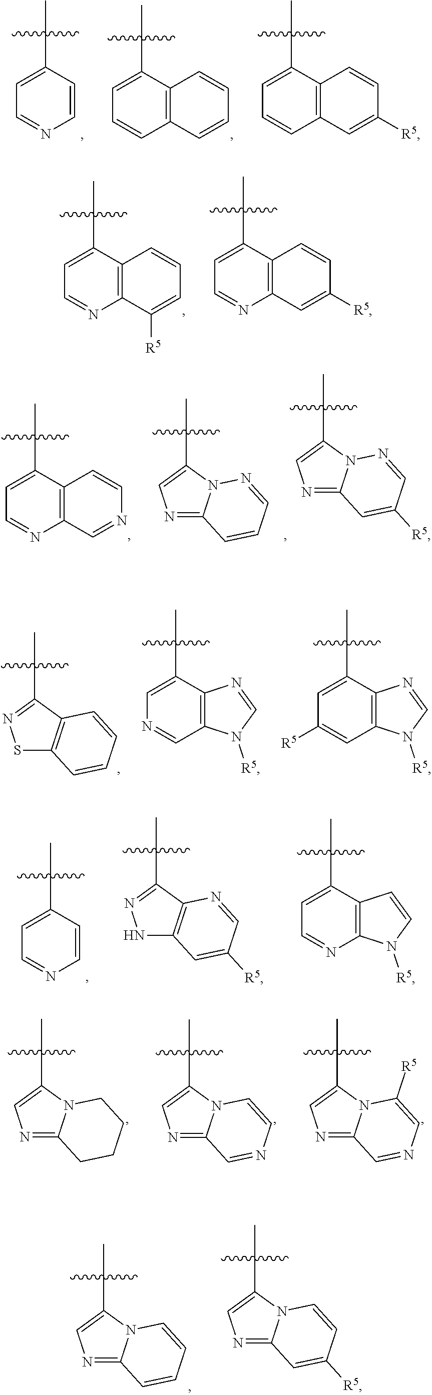

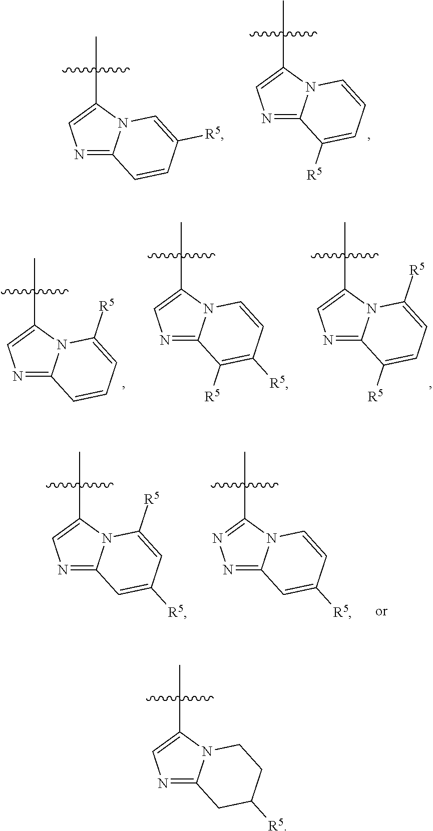

In embodiments, for the compound of Formula (I), or a pharmaceutically acceptable salt thereof, R¹ is:

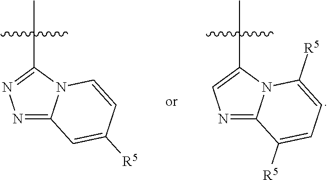

In embodiments, for the compound of Formula (I), or a pharmaceutically acceptable salt thereof, R¹ is:

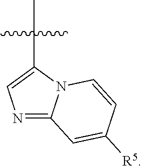

In embodiments, for the compound of Formula (I), or a pharmaceutically acceptable salt thereof, each R⁵ is independently halo, cyano, C₁₋₃alkoxy, C₁₋₃haloalkyl, or C₁₋₃alkyl.

In embodiments, for the compound of Formula (I), or a pharmaceutically acceptable salt thereof, each R⁵ is independently halo, cyano, C₁₋₃alkoxy, C₁₋₃haloalkyl, OH, or C₁₋₃alkyl.

In embodiments, for the compound of Formula (I), or a pharmaceutically acceptable salt thereof, each R⁵ is independently F, Cl, cyano, OCH₃, CF₃, or CH₃.

In embodiments, for the compound of Formula (I), or a pharmaceutically acceptable salt thereof, each R⁵ is independently F, Cl, cyano, OH, OCH₃, CF₃, or CH₃.

In embodiments, for the compound of Formula (I), or a pharmaceutically acceptable salt thereof, each R⁵ is independently F.

In embodiments, for the compound of Formula (I), or a pharmaceutically acceptable salt thereof, each R³ is independently halo, —OR^{O3a}, NR^{N3a}R^{N3b}, cyano, C₁₋₄alkyl, C₁₋₄haloalkyl, C₂alkynyl, or C₃₋₄cycloalkyl, wherein the C₁₋₄alkyl is optionally substituted by one or more R^{3a}; each R^{3a} is independently —OR^{O3a}, NR^{N3a}R^{N3b}, or 6-membered monocyclic heterocyclyl; and R^{O3a}, R^{N3a}, and R^{N3b} are each independently hydrogen or C₁₋₄alkyl.

In embodiments, for the compound of Formula (I), or a pharmaceutically acceptable salt thereof, each R³ is independently —C(O)R^{3a}, halo, —OR^{O3a}, NR^{N3a}R^{N3b}, cyano, C₁₋₄alkyl, C₁₋₄haloalkyl, C₂alkynyl, or C₃₋₄cycloalkyl, wherein the C₁₋₄alkyl is optionally substituted by one or more R^{3a}; each R^{3a} is independently —OR^{O3a}, NR^{N3a}R^{N3b}, or 6-membered monocyclic heterocyclyl; and R^{O3a}, R^{N3a}, and R^{N3b} are each independently hydrogen or C₁₋₄alkyl.

In embodiments, for the compound of Formula (I), or a pharmaceutically acceptable salt thereof, each R³ is independently —F, —Cl, —CH₃, —CF₃, —CH₂CH₃, —CH₂CH₂CH₃, —CH(CH₃), —CH₂CH(CH₃)₂, —CH₂N(CH₃)₂, —CH₂OH, —CH₂F, —CHF₂, —CH(OH)CH₃, —C(CH₃)₂OH, —CH₂OCH₃, —C≡CH, —N(CH₃)₂, —OCH₃, cyano, cyclopropyl, or

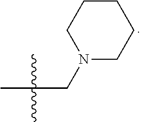 [SMILES: *CN1CCCCC1]

In embodiments, for the compound of Formula (I), or a pharmaceutically acceptable salt thereof, each R³ is independently —C(O)NH₂, —F, —Cl, —CH₃, —CF₃, —CH₂CH₃, —CH₂CH₂CH₃, —CH(CH₃), —CH₂CH(CH₃)₂, —CH₂N(CH₃)₂, —CH₂OH, —CH₂F, —CHF₂, —CH(OH)CH₃, —C(CH₃)₂OH, —CH₂OCH₃, —C≡CH, —N(CH₃)₂, —OCH₃, cyano, cyclopropyl, or

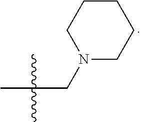 [SMILES: *CN1CCCCC1]

In embodiments, for the compound of Formula (I), or a pharmaceutically acceptable salt thereof, one of X², X³, and X⁴ is CR³, wherein R³ is C₁₋₃alkyl; and the remainder of X², X³, and X⁴ are CH.

In embodiments, for the compound of Formula (I), or a pharmaceutically acceptable salt thereof, each R³ is CH₃.

In embodiments, for the compound of Formula (I), or a pharmaceutically acceptable salt thereof, R⁶ is hydrogen or C₁₋₃alkyl.

In embodiments, for the compound of Formula (I), or a pharmaceutically acceptable salt thereof, R⁶ is hydrogen.

In embodiments, for the compound of Formula (I), or a pharmaceutically acceptable salt thereof, n is 0 or 1.

In embodiments, for the compound of Formula (I), or a pharmaceutically acceptable salt thereof, each R² is independently halo, C₁₋₄alkyl, C₁₋₄haloalkyl, or C₃₋₄cycloalkyl.

In embodiments, for the compound of Formula (I), or a pharmaceutically acceptable salt thereof, each R² is independently halo.

In embodiments, for the compound of Formula (I), or a pharmaceutically acceptable salt thereof, each R² is independently F, CF₃, CH₃ or cyclopropyl.

In embodiments, for the compound of Formula (I), or a pharmaceutically acceptable salt thereof, each R² is F.

In embodiments, for the compound of Formula (I), or a pharmaceutically acceptable salt thereof, n is 0.

In embodiments, the compound of Formula (I) is of Formula (II):

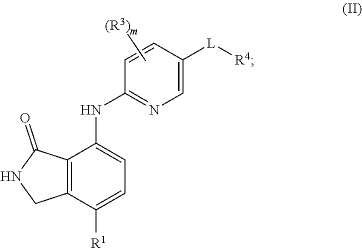

or a pharmaceutically acceptable salt thereof.

In embodiments, the compound of Formula (I) is of Formula (Ia), (Ib), (Ic), (Id), (Ie), (If), or (Ig):

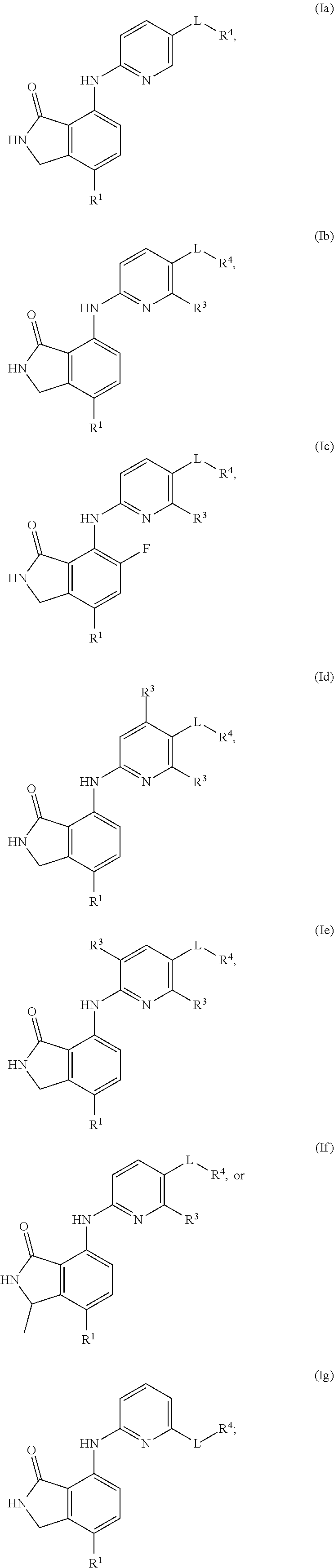

or a pharmaceutically acceptable salt thereof.

In embodiments, the compound of Formula (I) is of Formula (III):

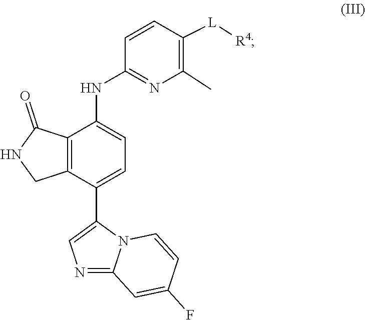

or a pharmaceutically acceptable salt thereof.

In embodiments, for the compound of Formula (I), (Ia), (Ib), (Ic), (Id), (Ie), (If), (Ig), (II), or (III), or a pharmaceutically acceptable salt thereof, R⁴ is:

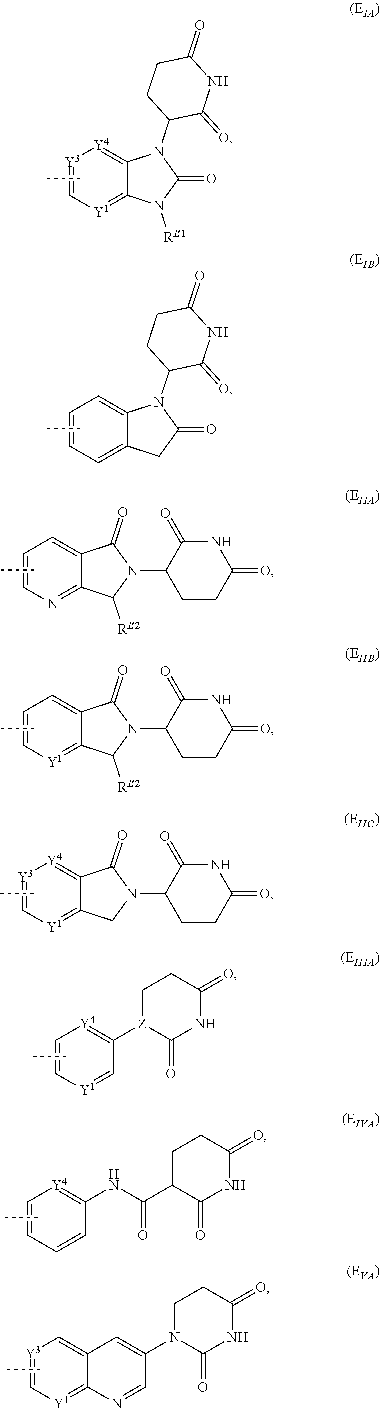

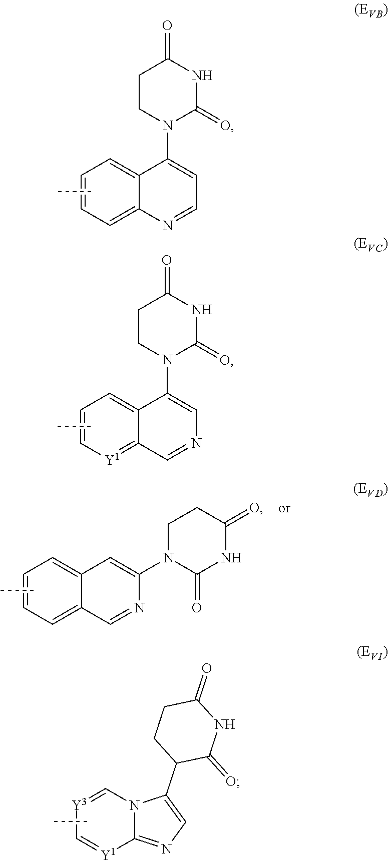

wherein:
Y¹, Y³, Y⁴ for E_{IA} is CR^{Y};
Y¹ for and E_{IIB} is CR^{Y};
Y¹, Y³, and Y⁴ for E_{IIC} is CR^{Y};
Y¹ for E_{IIIA} is CR^{Y} or N, and Y⁴ for E_{IIIA} is CR^{Y};
Y⁴ for E_{IVA} is CR^{Y};
Y¹ and Y³ for E_{VA} is CR^{Y};
Y¹ for E_{VC} is CR^{Y};
Y¹ and Y³ for E_{VI} is CR^{Y}.

In embodiments, for the compound of Formula (I), (Ia), (Ib), (Ic), (Id), (Ie), (If), (Ig), (II), or (III), or a pharmaceutically acceptable salt thereof, R⁴ is:

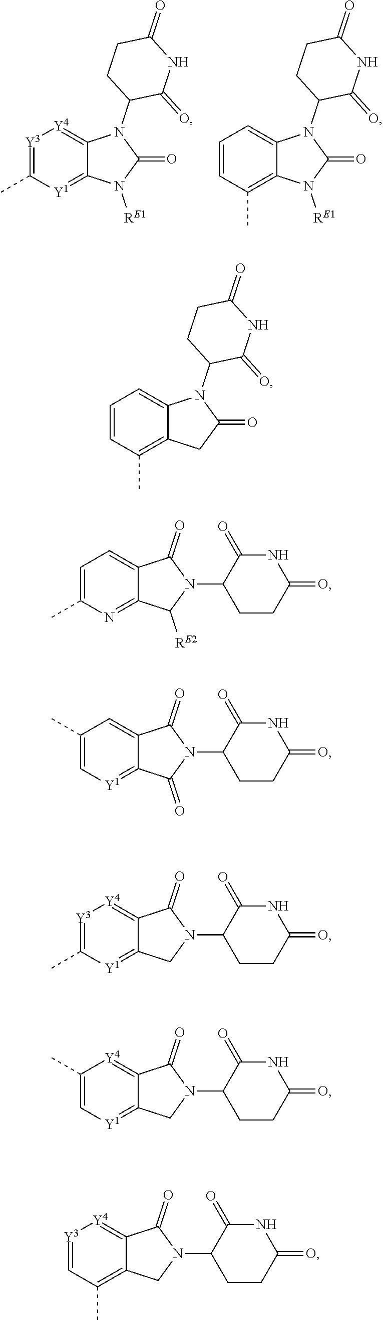

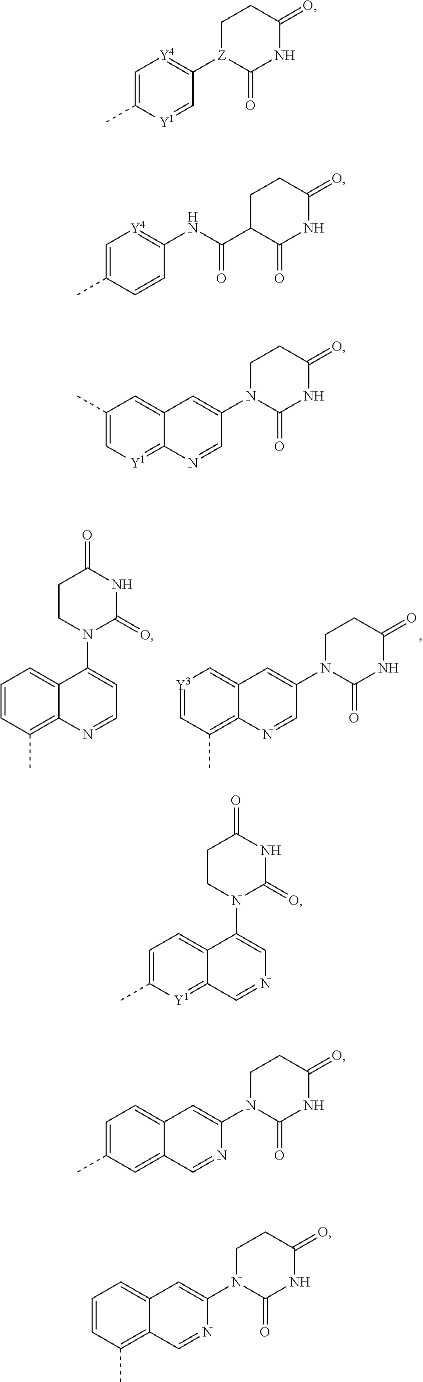

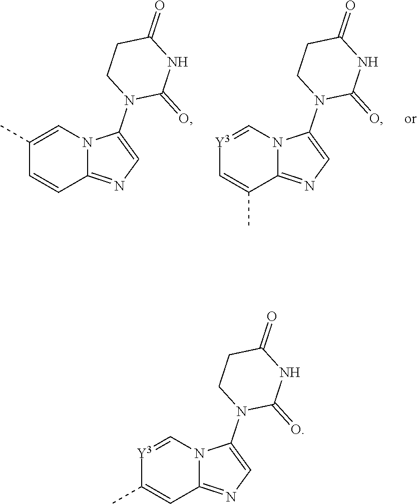

In embodiments, for the compound of Formula (I), (Ia), (Ib), (Ic), (Id), (Ie), (If), (Ig), (II), or (III), or a pharmaceutically acceptable salt thereof, each R^{Y} is independently hydrogen, halo, C₁₋₃alkyl, or C₁₋₃alkoxy.

In embodiments, for the compound of Formula (I), (Ia), (Ib), (Ic), (Id), (Ie), (If), (Ig), (II), or (III), or a pharmaceutically acceptable salt thereof, each R^{Y} is independently hydrogen, F, Cl, CH₃, or OCH₃.

In embodiments, for the compound of Formula (I), (Ia), (Ib), (Ic), (Id), (Ie), (If), (Ig), (II), or (III), or a pharmaceutically acceptable salt thereof, R⁴ is:

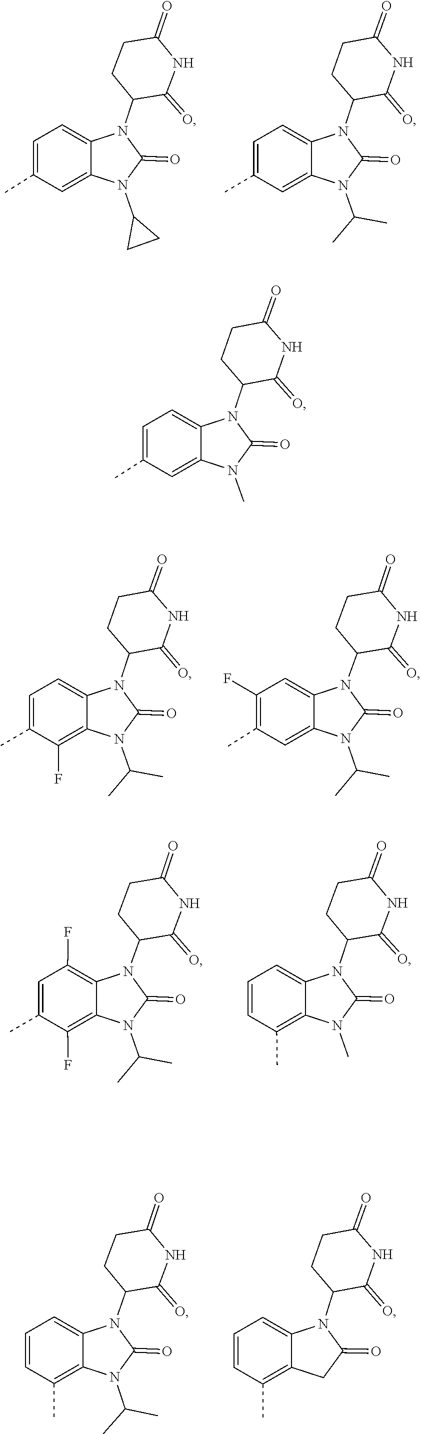

[chemical structure]

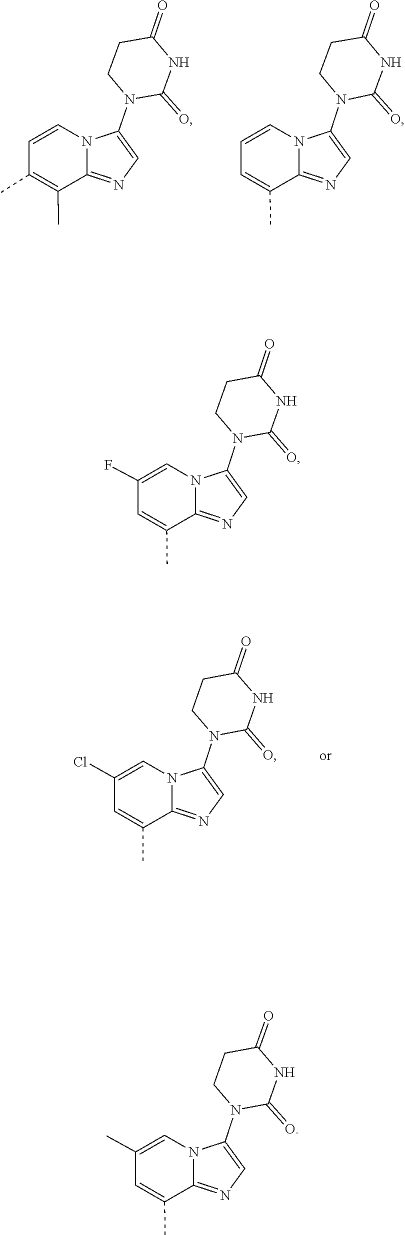

In embodiments, for the compound of Formula (I), (Ia), (Ib), (Ic), (Id), (Ie), (If), (Ig), (II), or (III), or a pharmaceutically acceptable salt thereof, R⁴ is:

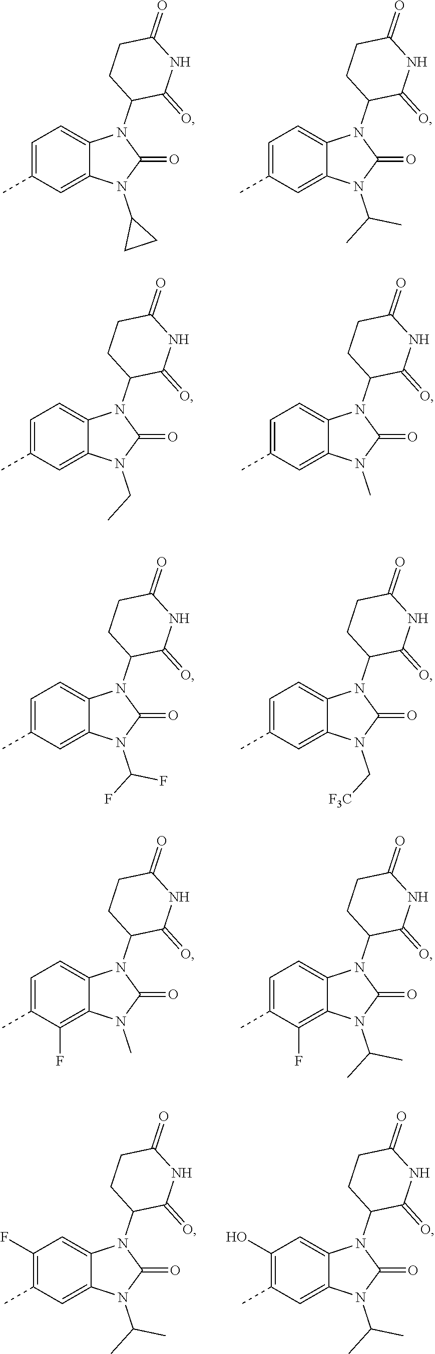

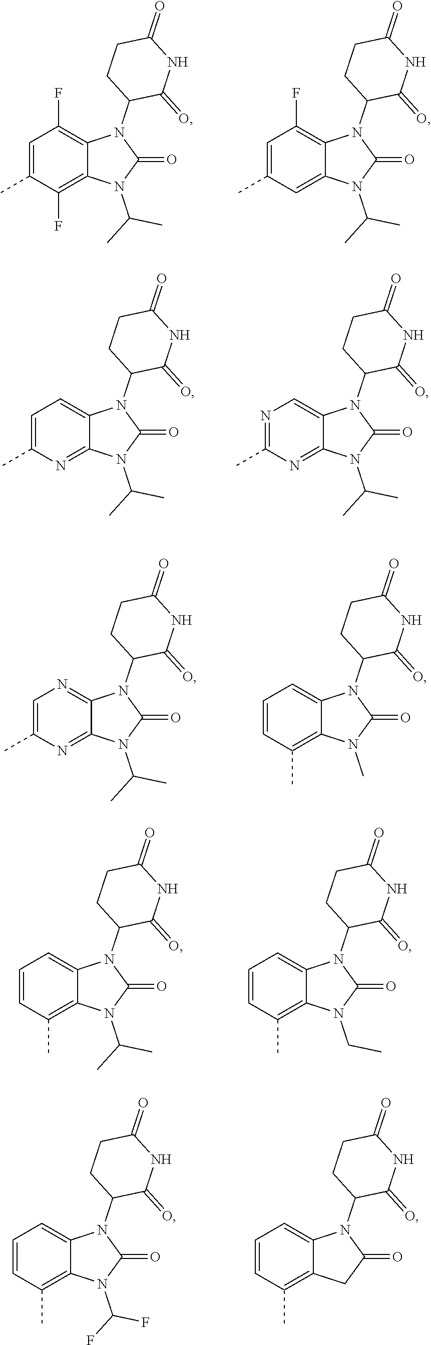

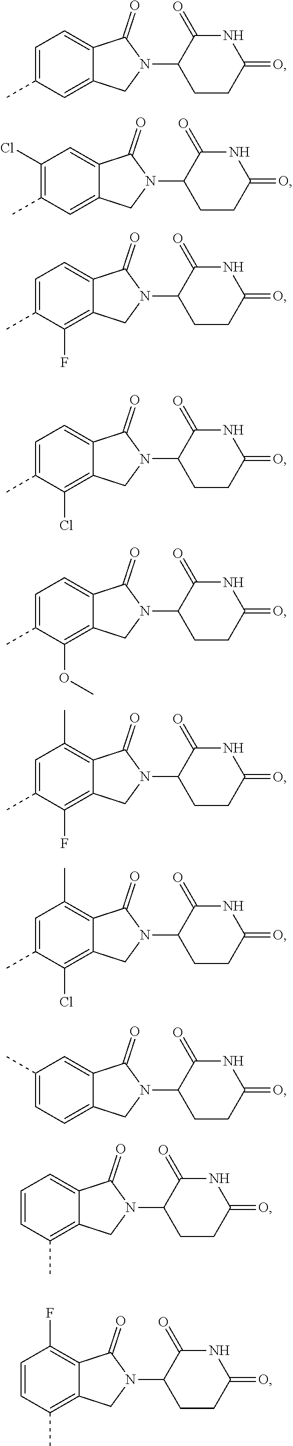

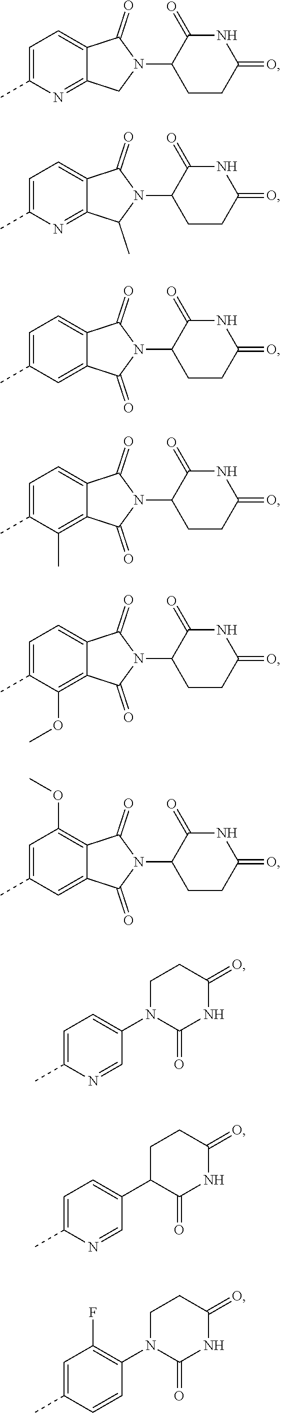

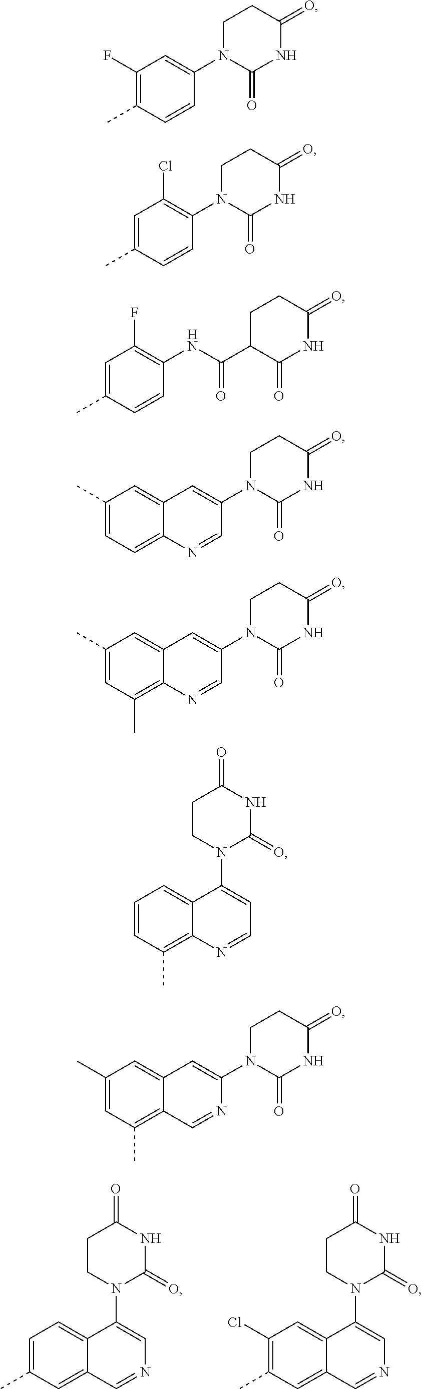

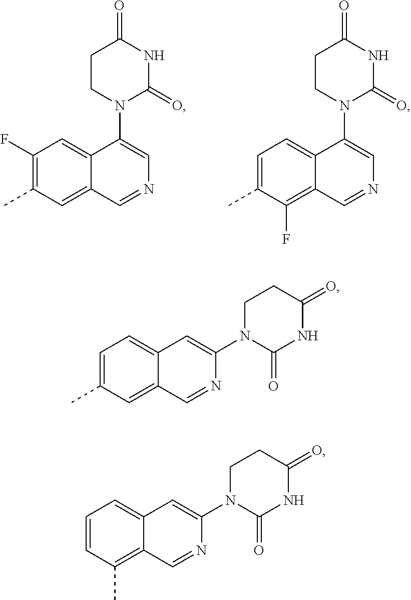

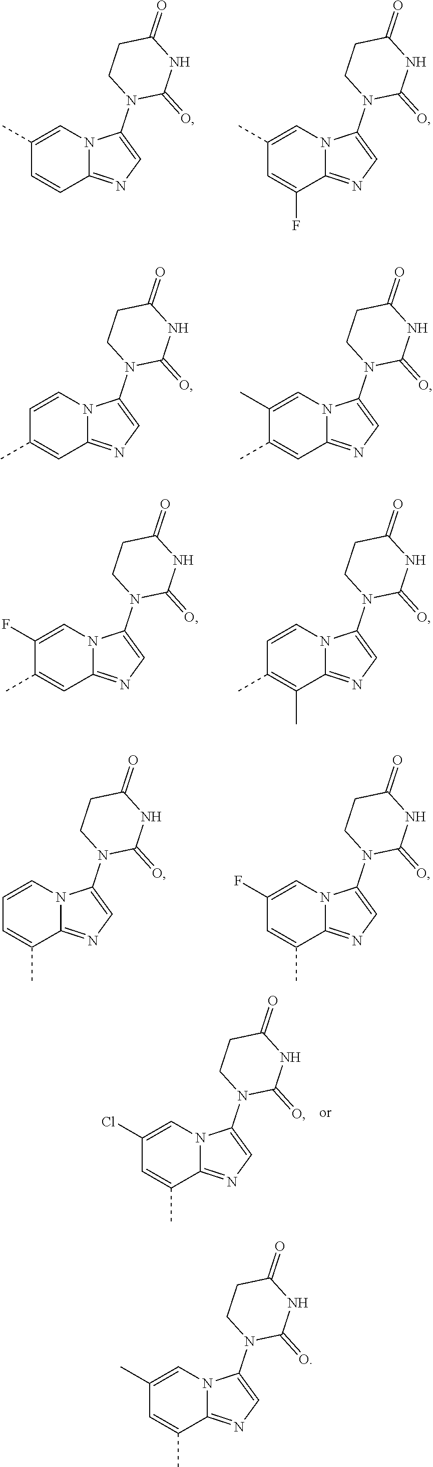

In embodiments, for the compound of Formula (I), (Ia), (Ib), (Ic), (Id), (Ie), (If), (Ig), (II), or (III), or a pharmaceutically acceptable salt thereof, R⁴ is:

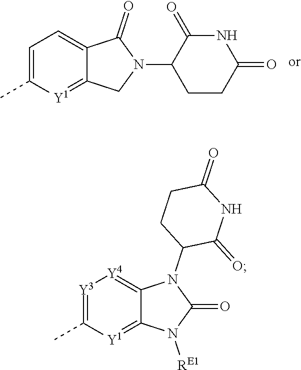

wherein:
R^{E1} is C₁₋₃alkyl or C₃₋₄cycloalkyl;
Y¹ is N or CR^{Y};
Y³ and Y⁴ are each independently CR^{Y};
each R^{Y} is independently hydrogen or halo.

In embodiments, for the compound of Formula (I), (Ia), (Ib), (Ic), (Id), (Ie), (If), (Ig), (II), or (III), or a pharmaceutically acceptable salt thereof, R⁴ is:

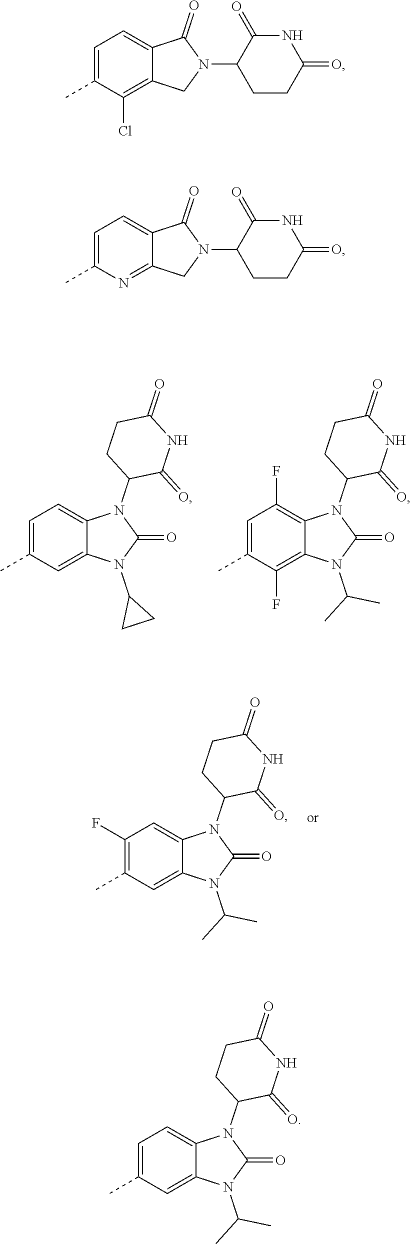

In embodiments, for the compound of Formula (I), (Ia), (Ib), (Ic), (Id), (Ie), (If), (Ig), (II), or (III), or a pharmaceutically acceptable salt thereof, L¹ is absent, CH₂, or CH₂O.

In embodiments, for the compound of Formula (I), (Ia), (Ib), (Ic), (Id), (Ie), (If), (Ig), (II), or (III), or a pharmaceutically acceptable salt thereof, L² is absent, CH₂, CH(CH)₃, C(O), or

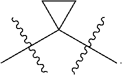 [SMILES: *C1(*)CC1]

In embodiments, for the compound of Formula (I), (Ia), (Ib), (Ic), (Id), (Ie), (If), (Ig), (II), or (III), or a pharmaceutically acceptable salt thereof, L³ is absent or O.

In embodiments, for the compound of Formula (I), (Ia), (Ib), (Ic), (Id), (Ie), (If), (Ig), (II), or (III), or a pharmaceutically acceptable salt thereof, L⁴ is absent.

In embodiments, for the compound of Formula (I), (Ia), (Ib), (Ic), (Id), (Ie), (If), (Ig), (II), or (III), or a pharmaceutically acceptable salt thereof, Ring A is a 6-membered monocylic carbocyclyl, 6- to 10-membered heterocyclyl, or 5-membered monocyclic heteroaryl, each of which is optionally substituted by one or two R^{L}.

In embodiments, for the compound of Formula (I), (Ia), (Ib), (Ic), (Id), (Ie), (If), (Ig), (II), or (III), or a pharmaceutically acceptable salt thereof, Ring A is cyclohexyl, cyclohexenyl, piperdinyl, piperazinyl, morpholinyl, 2,7-diazaspiro[3.5]nonanyl, 5-oxa-2,8-diazaspiro[3.5]nonanyl, 2-azabicyclo[2.2.1]heptanyl, 2,5-diazabicyclo[2.2.1]heptanyl, or pyrazolyl, each of which is optionally substituted by one or two R^{L}.

In embodiments, for the compound of Formula (I), (Ia), (Ib), (Ic), (Id), (Ie), (If), (Ig), (II), or (III), or a pharmaceutically acceptable salt thereof, Ring B is absent, a 6-membered monocylic carbocyclyl, or a 4- to 12-membered heterocyclyl optionally substituted by one or two R^{L}.

In embodiments, for the compound of Formula (I), (Ia), (Ib), (Ic), (Id), (Ie), (If), (Ig), (II), or (III), or a pharmaceutically acceptable salt thereof, Ring B is cyclohexyl, azetidinyl, pyrrolidinyl, piperdinyl, piperazinyl, 2-azaspiro[3.3]heptanyl, 3-azaspiro[5.5]undecanyl, 7-azaspiro[3.5]nonanyl, or 2-azaspiro[3.5]nonanyl, each of which is optionally substituted by one or two R^{L}.

In embodiments, for the compound of Formula (I), (Ia), (Ib), (Ic), (Id), (Ie), (If), (Ig), (II), or (III), or a pharmaceutically acceptable salt thereof, Ring C is 6-membered monocyclic heterocyclyl.

In embodiments, for the compound of Formula (I), (Ia), (Ib), (Ic), (Id), (Ie), (If), (Ig), (II), or (III), or a pharmaceutically acceptable salt thereof, Ring C is piperdinyl.

In embodiments, for the compound of Formula (I), (Ia), (Ib), (Ic), (Id), (Ie), (If), (Ig), (II), or (III), or a pharmaceutically acceptable salt thereof, each R^{L} is independently halo or C₁₋₃alkyl.

In embodiments, for the compound of Formula (I), (Ia), (Ib), (Ic), (Id), (Ie), (If), (Ig), (II), or (III), or a pharmaceutically acceptable salt thereof, each R^{L} is independently F or CH₃.

In embodiments, for the compound of Formula (I), (Ia), (Ib), (Ic), (Id), (Ie), (If), (Ig), (II), or (III), or a pharmaceutically acceptable salt thereof, L is:

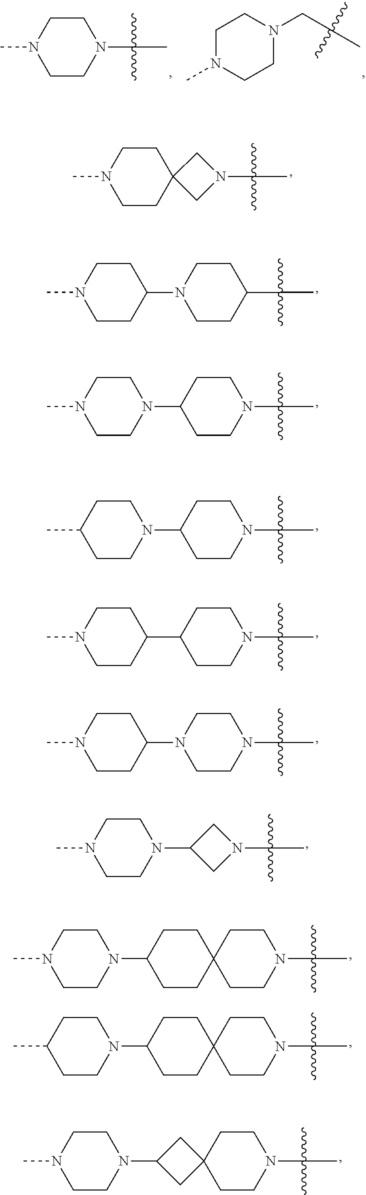

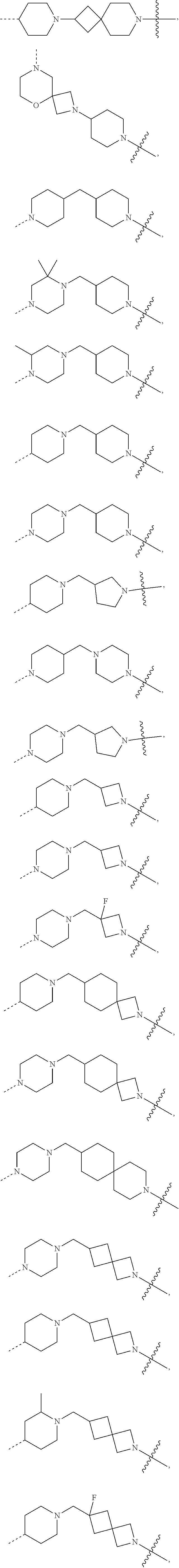

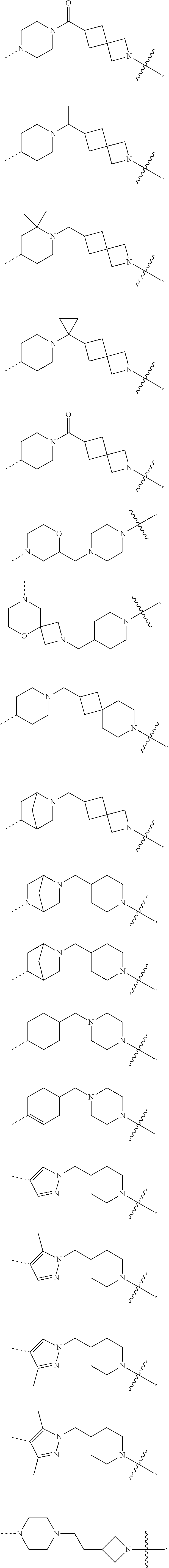

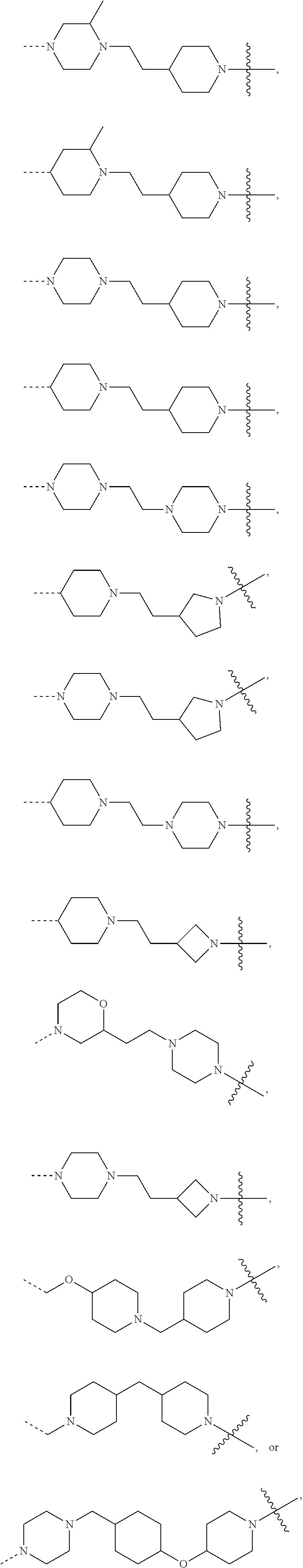

wherein

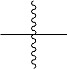

indicates the attachment point to R⁴.

In embodiments, for the compound of Formula (I), (Ia), (Ib), (Ic), (Id), (Ie), (If), (Ig), (II), or (III), or a pharmaceutically acceptable salt thereof, L is:

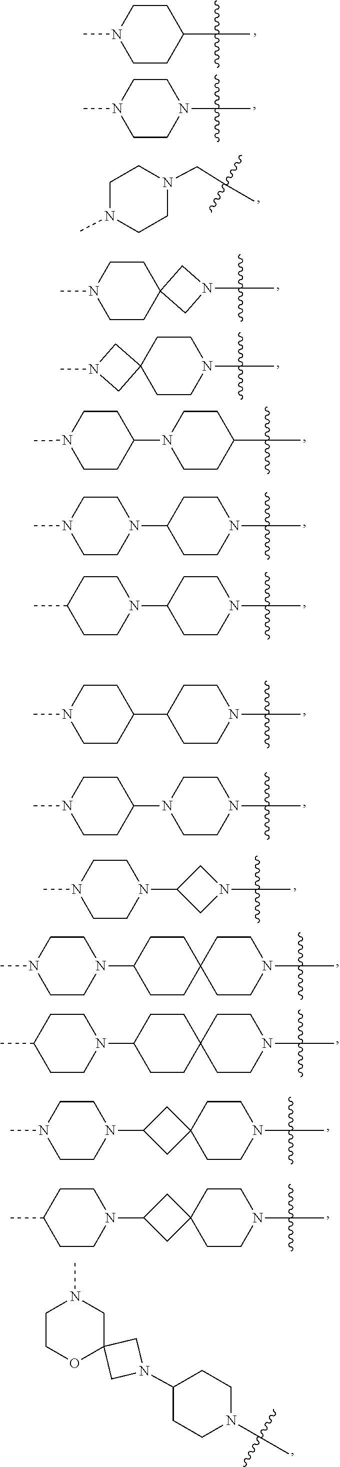

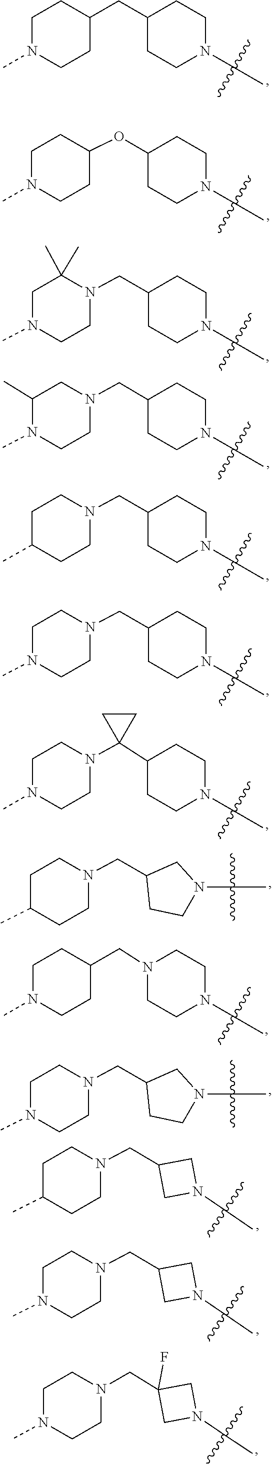

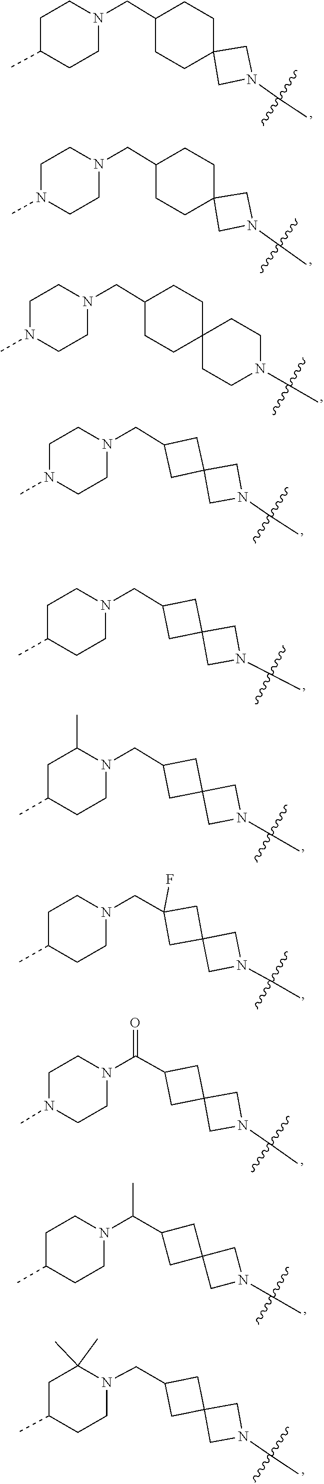

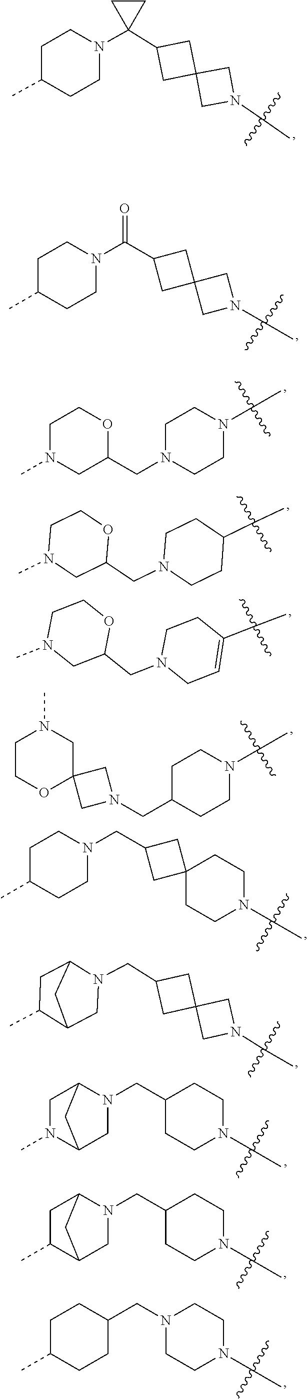

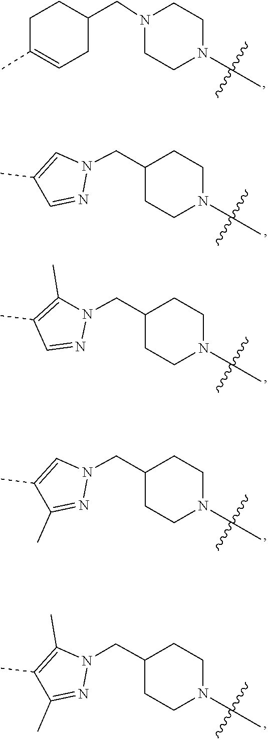

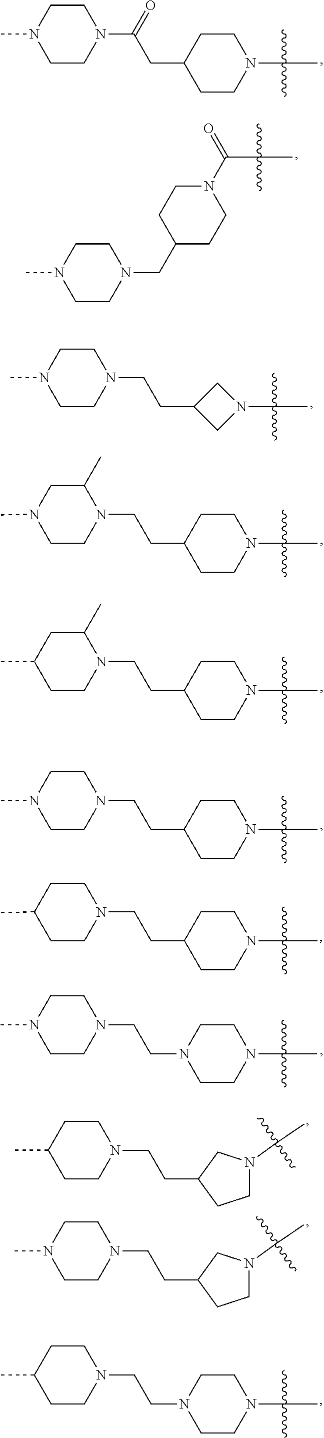

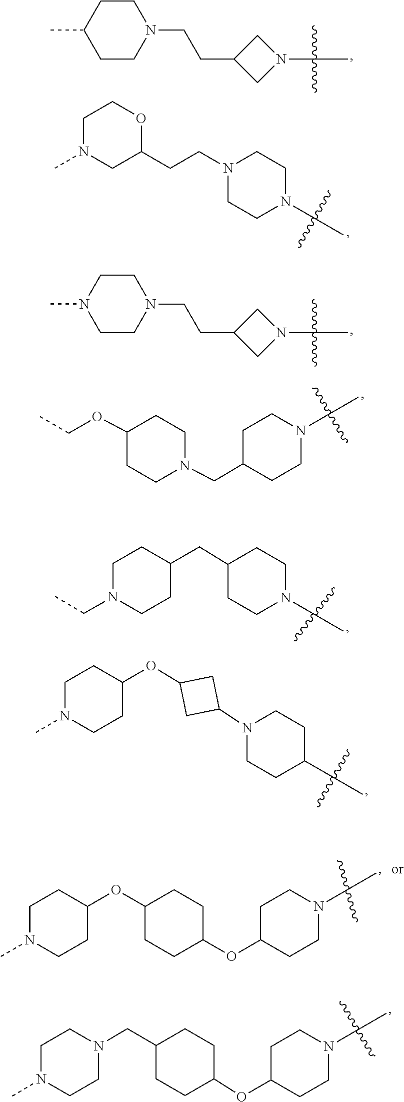

wherein

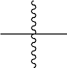

indicates the attachment point to R⁴.

In embodiments, for the compound of Formula (I), (Ia), (Ib), (Ic), (Id), (Ie), (If), (Ig), (II), or (III), or a pharmaceutically acceptable salt thereof, L is:

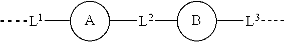

wherein:
Ring A and Ring B are each absent or a 4- to 10-membered carbocyclyl, 4- to 12-membered heterocyclyl, phenyl, or 5- to 10-membered heteroaryl, each of which are optionally substituted by one or more R^{L};
each R^{L} is independently halo, C₁₋₄alkyl, or C₁₋₄haloalkyl;
L¹, L², and L³ are each absent or C₁₋₄alkylene, wherein the C₁₋₄alkylene is optionally substituted by one or more R^{L1}, and wherein one or more C in the C₁₋₄alkylene is optionally replaced by O;
each R^{L1} is independently halo, or two R^{L1} groups, together with the carbon to which they are attached, form oxo or C₃₋₆cycloalkyl.

In embodiments, for the compound of Formula (I), (Ia), (Ib), (Ic), (Id), (Ie), (If), (Ig), (II), or (III), or a pharmaceutically acceptable salt thereof, L is:

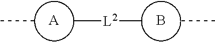

wherein Rings A and B are each independently 6-membered monocyclic heterocyclyl; and L² is absent or CH₂.

In embodiments, for the compound of Formula (I), (Ia), (Ib), (Ic), (Id), (Ie), (If), (Ig), (II), or (III), or a pharmaceutically acceptable salt thereof, Ring A is piperdinyl or piperazinyl; and Ring B is piperdinyl.

In embodiments, for the compound of Formula (I), (Ia), (Ib), (Ic), (Id), (Ie), (If), (Ig), (II), or (III), or a pharmaceutically acceptable salt thereof, L is:

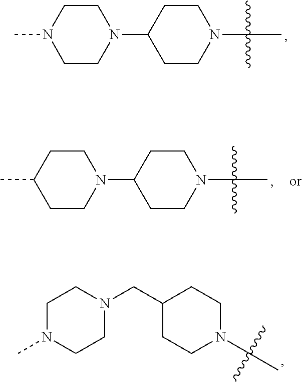

wherein

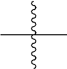

indicates the attachment point to R⁴.

In embodiments, the compound of Formula (I) is of Formula (IV):

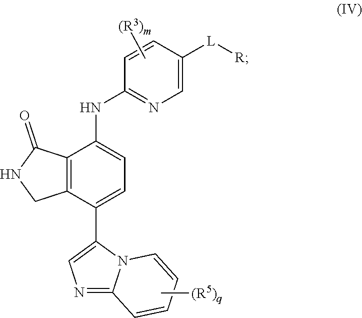

or a pharmaceutically acceptable salt thereof, wherein:
m is 0 or 1;
q is 0, 1, or 2;
each R³ and R⁵ is independently halo or C₁₋₂alkyl;
L is

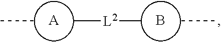

wherein:
   Rings A and B are each independently 6-membered monocyclic heterocyclyl;
   L² is absent or CH₂;
R⁴ is

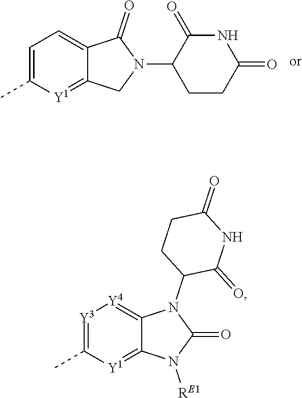

wherein
   R^{E1} is C₁₋₃alkyl or C₃₋₄cycloalkyl;
   Y is N or CR^{Y};
   Y³ and Y⁴ are each independently CR^{Y};
   each R^{Y} is independently hydrogen or halo.

In embodiments, for the compound of Formula (IV), or a pharmaceutically acceptable salt thereof, m is 1; q is 1 or 2; R³ is CH₃; and each R⁵ is F.

In embodiments, the compound of Formula (I) or (IV) is of Formula (III):

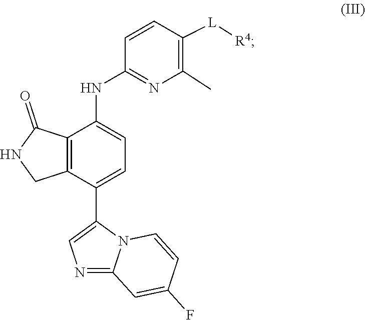

or a pharmaceutically acceptable salt thereof.

In embodiments, for the compound of Formula (III) or (IV), or a pharmaceutically acceptable salt thereof, Ring A and Ring B are each independently piperazinyl or piperdinyl.

In embodiments, for the compound of Formula (III) or (IV), or a pharmaceutically acceptable salt thereof, Ring A is piperazinyl and Ring B is piperdinyl.

In embodiments, for the compound of Formula (III) or (IV), or a pharmaceutically acceptable salt thereof, L² is —CH₂—.

In embodiments, for the compound of Formula (III) or (IV), or a pharmaceutically acceptable salt thereof, R⁴ is

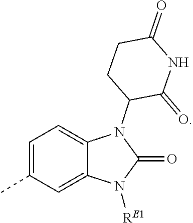

In embodiments, for the compound of Formula (III) or (IV), or a pharmaceutically acceptable salt thereof, R_{E1} is —CH(CH₃)₂.

In embodiments, provided herein is a compound of Formula (V):

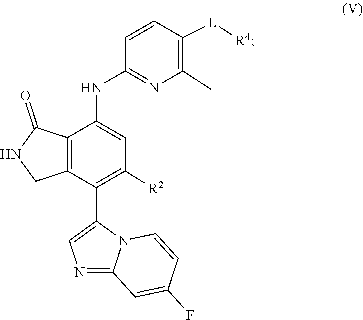

or a pharmaceutically acceptable salt thereof, wherein:
R² is H or C₁₋₄alkyl;
L is

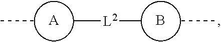

wherein:
   Rings A and B are each independently 6-membered monocyclic heterocyclyl;
   L² is absent or CH₂;
R⁴ is

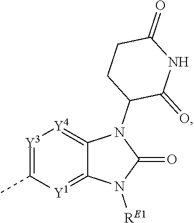

wherein
   R^{E1} is C₁₋₃alkyl or C₃₋₄cycloalkyl;
   Y¹ is N or CR^{Y};
   Y³ and Y⁴ are each independently CR^{Y};
   each R^{Y} is independently hydrogen or halo.

In embodiments, for the compound of Formula (V), R² is H or CH₃.

In embodiments, provided herein is a compound of Formula (III):

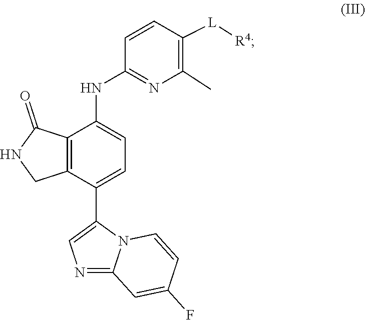

or a pharmaceutically acceptable salt thereof, wherein:
L is

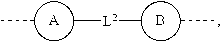

wherein:
   Rings A and B are each independently 6-membered monocyclic heterocyclyl;
   L² is absent or CH₂;
R⁴ is

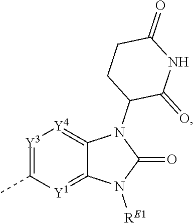

wherein
   R^{E1} is C₁₋₃alkyl or C₃₋₄cycloalkyl;
   Y is N or CR^{Y};
   Y³ and Y⁴ are each independently CR^{Y};
   each R^{Y} is independently hydrogen or halo.

In embodiments, for the compound of Formula (III) or (V), or a pharmaceutically acceptable salt thereof, Ring A and Ring B are each independently piperazinyl or piperdinyl.

In embodiments, for the compound of Formula (III) or (V), or a pharmaceutically acceptable salt thereof, Ring A is piperazinyl and Ring B is piperdinyl.

In embodiments, for the compound of Formula (III) or (V), or a pharmaceutically acceptable salt thereof, L² is —CH₂—.

In embodiments, for the compound of Formula (III) or (V), or a pharmaceutically acceptable salt thereof, L² is absent.

In embodiments, for the compound of Formula (III) or (IV), or a pharmaceutically acceptable salt thereof, R^{E1} is —CH(CH₃)₂.

In embodiments, for the compound of Formula (III) or (IV), or a pharmaceutically acceptable salt thereof, R^{E1} is cyclopropyl.

In embodiments, the compound is any one of examples 1 to 294, or a pharmaceutically acceptable salt thereof.

In embodiments, the compound is any one of examples 1 to 457, or a pharmaceutically acceptable salt thereof.

In embodiments, the compounds is any one of Examples 1, 247, 395, 245, 228, 182, or 73, or a pharmaceutically acceptable salt thereof.

In embodiments, the compound is a compound selected from the table below, or a pharmaceutically acceptable salt thereof:

| Ex. No. | Structure |
|---|---|
| 1 | 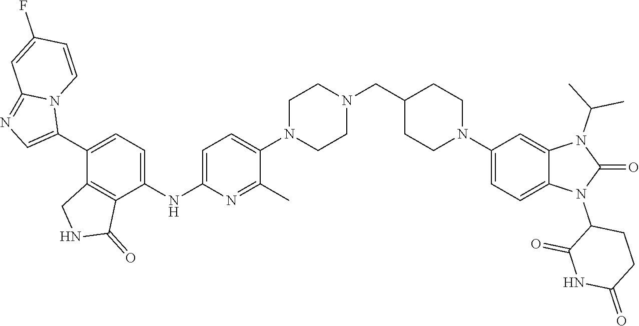 [SMILES: Cc1nc(Nc2ccc(-c3cnc4cc(F)ccn34)c3c2C(=O)NC3)ccc1N1CCN(CC2CCN(c3ccc4c(c3)n(C(C)C)c(=O)n4C3CCC(=O)NC3=O)CC2)CC1] |
| 2 | 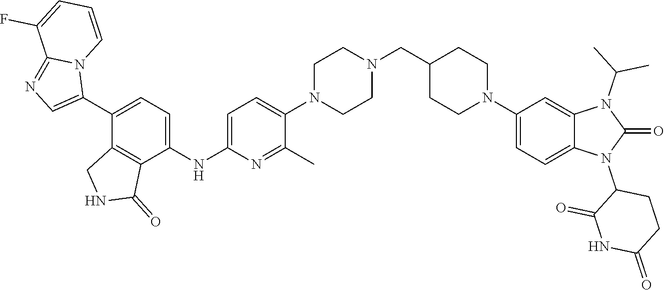 [SMILES: Cc1nc(Nc2ccc(-c3cnc4c(F)cccn34)c3c2C(=O)NC3)ccc1N1CCN(CC2CCN(c3ccc4c(c3)n(C(C)C)c(=O)n4C3CCC(=O)NC3=O)CC2)CC1] |
| 3 | 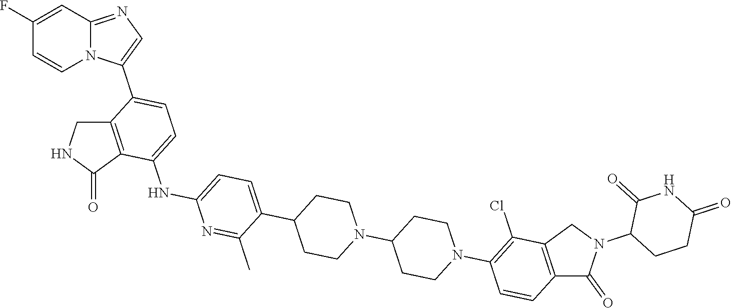 [SMILES: Cc1nc(Nc2ccc(-c3cnc4cc(F)ccn34)c3c2C(=O)NC3)ccc1C1CCN(C2CCN(c3ccc4c(c3Cl)CN(C3CCC(=O)NC3=O)C4=O)CC2)CC1] |
| 4 | 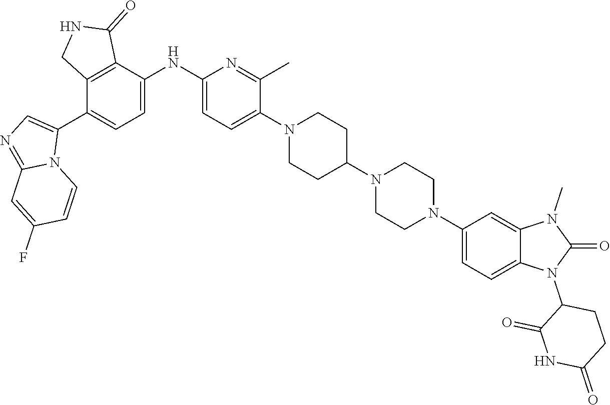 [SMILES: Cc1nc(Nc2ccc(-c3cnc4cc(F)ccn34)c3c2C(=O)NC3)ccc1N1CCC(N2CCN(c3ccc4c(c3)n(C)c(=O)n4C3CCC(=O)NC3=O)CC2)CC1] |
| 5 | 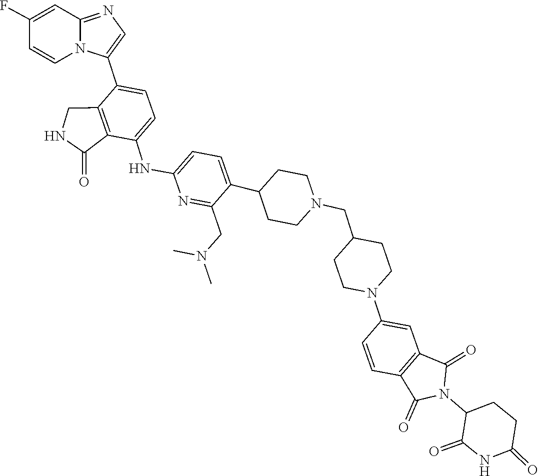 [SMILES: CN(C)Cc1nc(Nc2ccc(-c3cnc4cc(F)ccn34)c3c2C(=O)NC3)ccc1C1CCN(CC2CCN(c3ccc4c(c3)C(=O)N(C3CCC(=O)NC3=O)C4=O)CC2)CC1] |
| 6 | 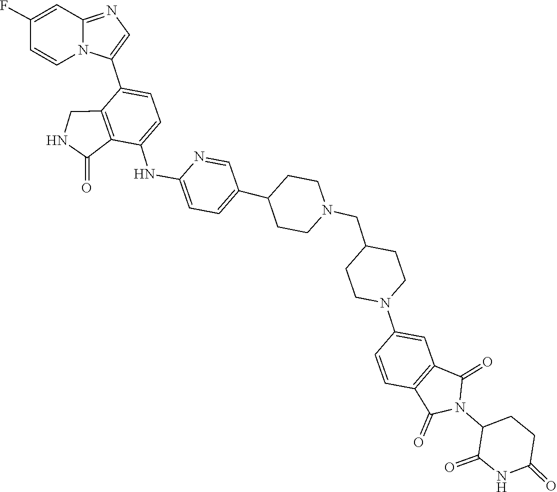 [SMILES: O=C1CCC(N2C(=O)c3ccc(N4CCC(CN5CCC(c6ccc(Nc7ccc(-c8cnc9cc(F)ccn89)c8c7C(=O)NC8)nc6)CC5)CC4)cc3C2=O)C(=O)N1] |
| 7 | 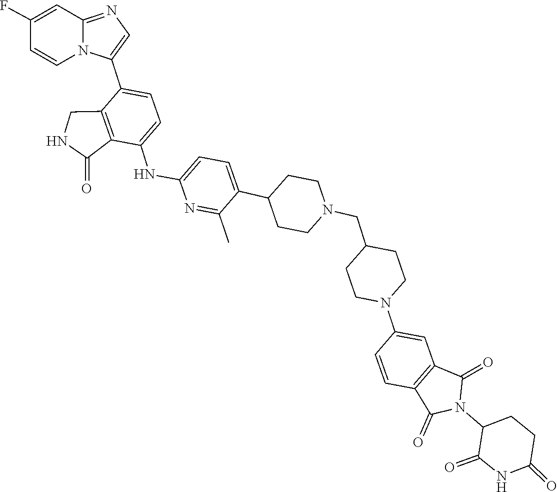 [SMILES: Cc1nc(Nc2ccc(-c3cnc4cc(F)ccn34)c3c2C(=O)NC3)ccc1C1CCN(CC2CCN(c3ccc4c(c3)C(=O)N(C3CCC(=O)NC3=O)C4=O)CC2)CC1] |
| 8 | 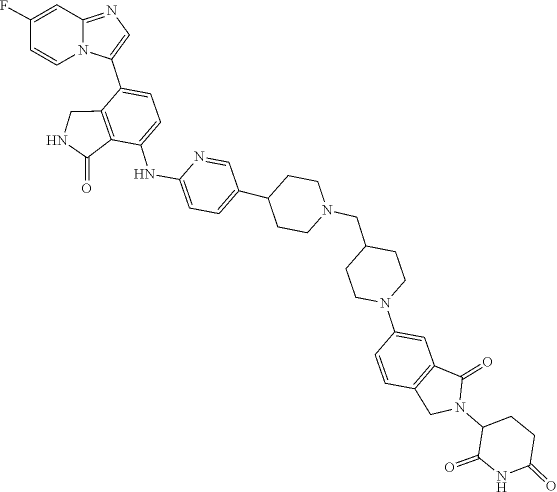 [SMILES: O=C1CCC(N2Cc3ccc(N4CCC(CN5CCC(c6ccc(Nc7ccc(-c8cnc9cc(F)ccn89)c8c7C(=O)NC8)nc6)CC5)CC4)cc3C2=O)C(=O)N1] |
| 9 | 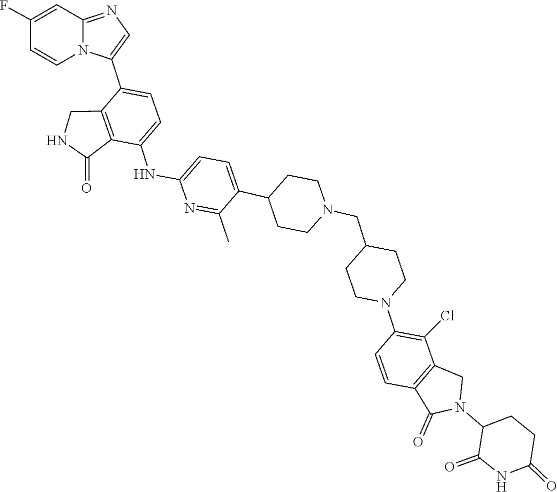 [SMILES: Cc1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 10 | 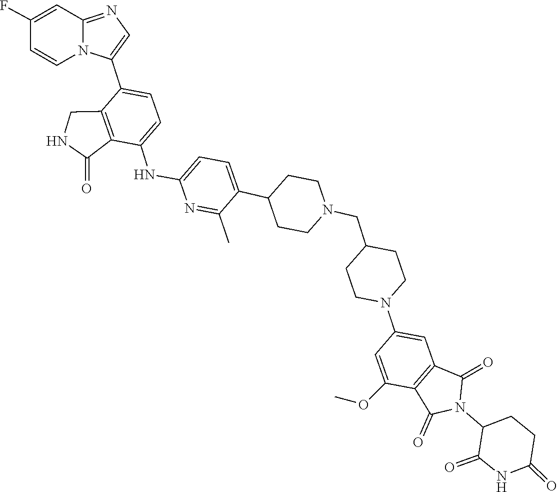 [SMILES: COc1cc(N2CCC(CN3CCC(c4ccc(Nc5ccc(-c6cnc7cc(F)ccn67)c6c5C(=O)NC6)nc4C)CC3)CC2)cc2c1C(=O)N(C1CCC(=O)NC1=O)C2=O] |
| 11 | 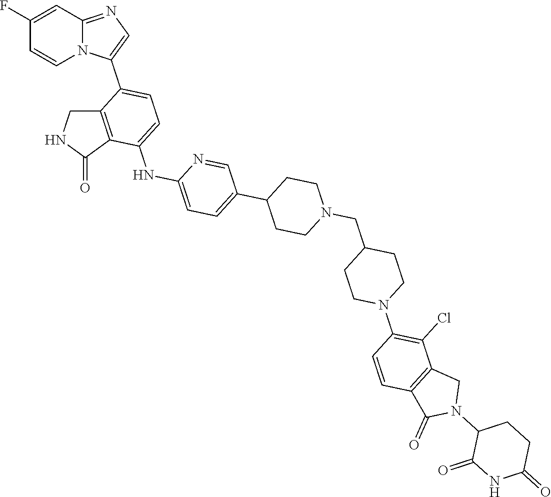 [SMILES: O=C1CCC(N2Cc3c(ccc(N4CCC(CN5CCC(c6ccc(Nc7ccc(-c8cnc9cc(F)ccn89)c8c7C(=O)NC8)nc6)CC5)CC4)c3Cl)C2=O)C(=O)N1] |
| 12 | 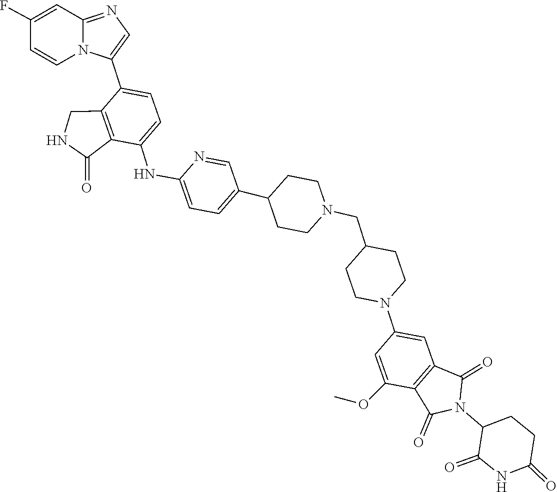 [SMILES: COc1cc(N2CCC(CN3CCC(c4ccc(Nc5ccc(-c6cnc7cc(F)ccn67)c6c5C(=O)NC6)nc4)CC3)CC2)cc2c1C(=O)N(C1CCC(=O)NC1=O)C2=O] |
| 13 | 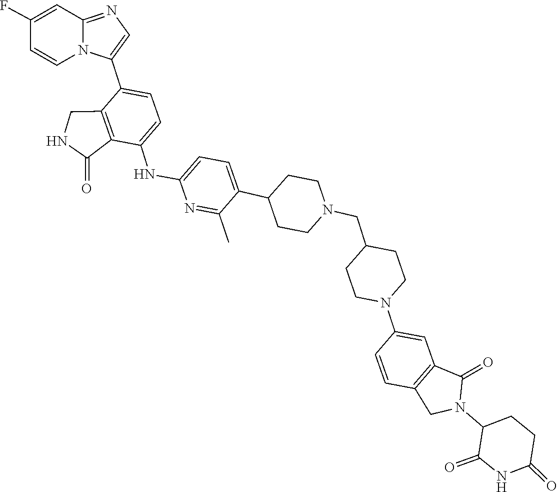 [SMILES: Cc1nc(Nc2ccc(-c3cnc4cc(F)ccn34)c3c2C(=O)NC3)ccc1C1CCN(CC2CCN(c3ccc4c(c3)C(=O)N(C3CCC(=O)NC3=O)C4)CC2)CC1] |
| 14 | 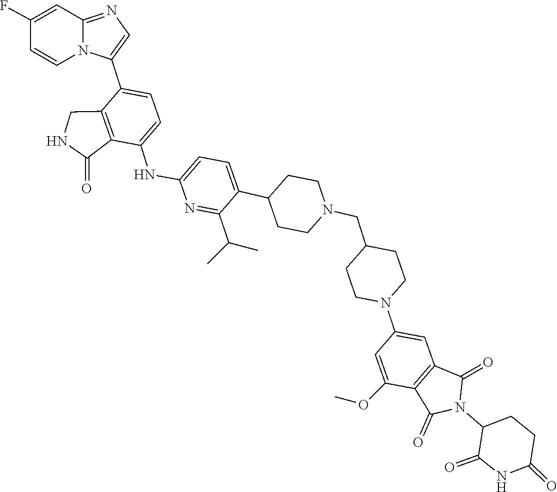 [SMILES: COc1cc(N2CCC(CN3CCC(c4ccc(Nc5ccc(-c6cnc7cc(F)ccn67)c6c5C(=O)NC6)nc4C(C)C)CC3)CC2)cc2c1C(=O)N(C1CCC(=O)NC1=O)C2=O] |
| 15 | 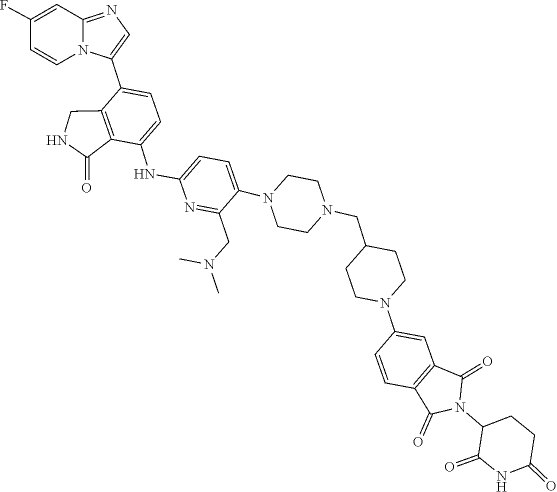 [SMILES: CN(C)Cc1nc(Nc2ccc(-c3cnc4cc(F)ccn34)c3c2C(=O)NC3)ccc1N1CCN(CC2CCN(c3ccc4c(c3)C(=O)N(C3CCC(=O)NC3=O)C4=O)CC2)CC1] |
| 16 | 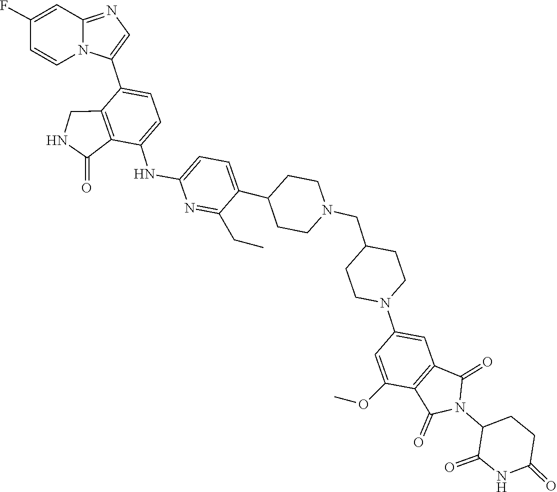 [SMILES: CCc1nc(Nc2ccc(-c3cnc4cc(F)ccn34)c3c2C(=O)NC3)ccc1C1CCN(CC2CCN(c3cc(OC)c4c(c3)C(=O)N(C3CCC(=O)NC3=O)C4=O)CC2)CC1] |
| 17 | 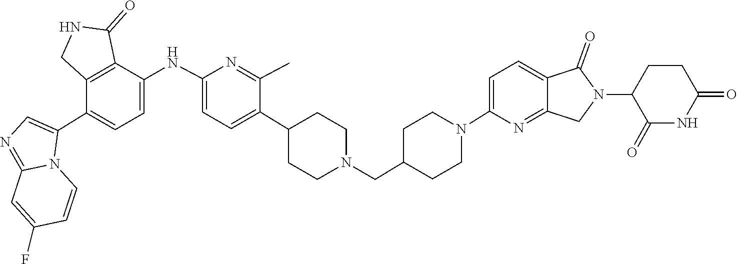 [SMILES: Cc1nc(Nc2ccc(-c3cnc4cc(F)ccn34)c3c2C(=O)NC3)ccc1C1CCN(CC2CCN(c3ccc4c(n3)CN(C3CCC(=O)NC3=O)C4=O)CC2)CC1] |
| 18 | 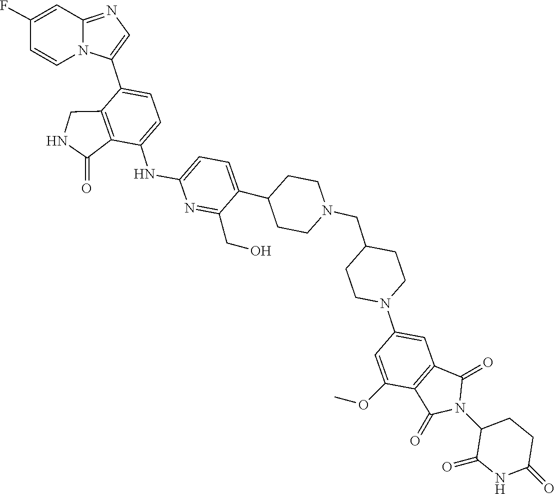 [SMILES: COc1cc(N2CCC(CN3CCC(c4ccc(Nc5ccc(-c6cnc7cc(F)ccn67)c6c5C(=O)NC6)nc4CO)CC3)CC2)cc2c1C(=O)N(C1CCC(=O)NC1=O)C2=O] |
| 19 | 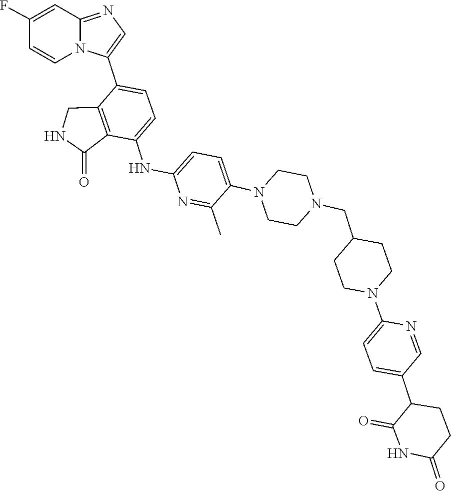 [SMILES: Cc1nc(Nc2ccc(-c3cnc4cc(F)ccn34)c3c2C(=O)NC3)ccc1N1CCN(CC2CCN(c3ccc(C4CCC(=O)NC4=O)cn3)CC2)CC1] |
| 20 | 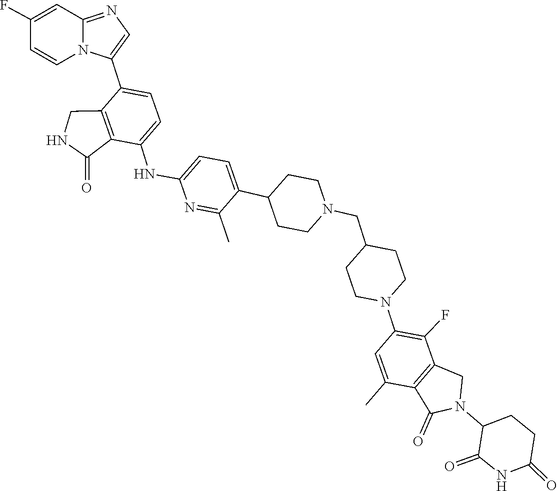 [SMILES: Cc1cc(N2CCC(CN3CCC(c4ccc(Nc5ccc(-c6cnc7cc(F)ccn67)c6c5C(=O)NC6)nc4C)CC3)CC2)c(F)c2c1C(=O)N(C1CCC(=O)NC1=O)C2] |
| 21 | 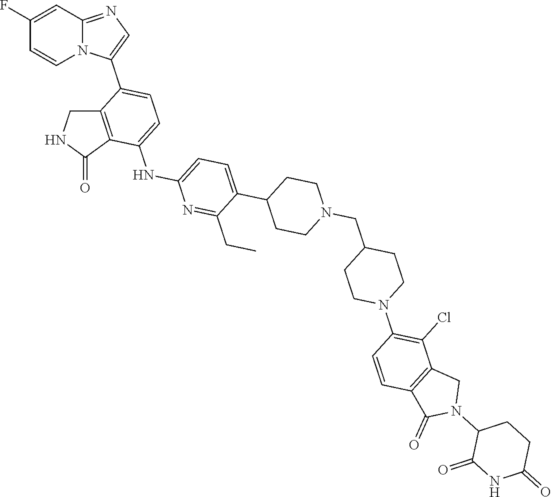 [SMILES: CCc1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 22 | 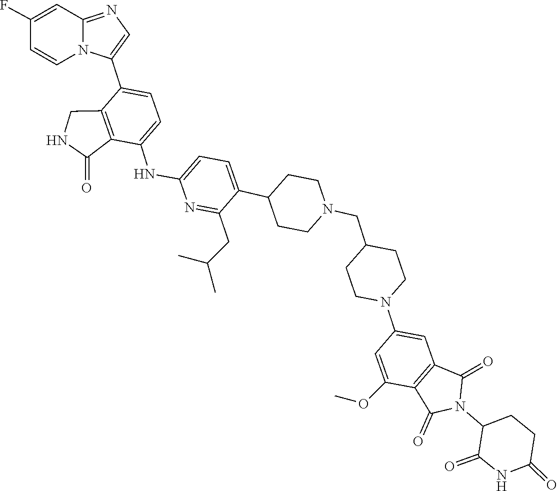 [SMILES: COc1cc(N2CCC(CN3CCC(c4ccc(Nc5ccc(-c6cnc7cc(F)ccn67)c6c5C(=O)NC6)nc4CC(C)C)CC3)CC2)cc2c1C(=O)N(C1CCC(=O)NC1=O)C2=O] |
| 23 | 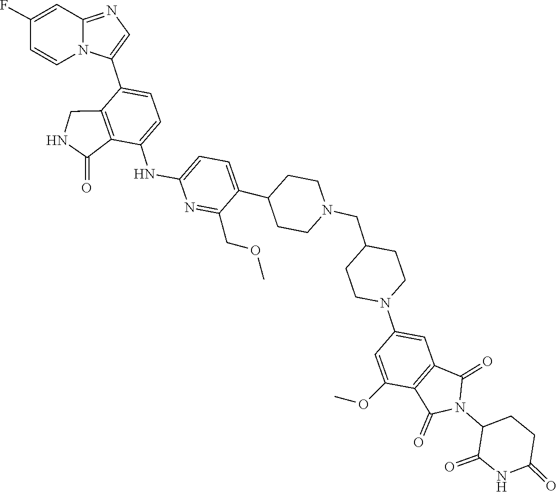 [SMILES: COCc1nc(Nc2ccc(-c3cnc4cc(F)ccn34)c3c2C(=O)NC3)ccc1C1CCN(CC2CCN(c3cc(OC)c4c(c3)C(=O)N(C3CCC(=O)NC3=O)C4=O)CC2)CC1] |
| 24 | 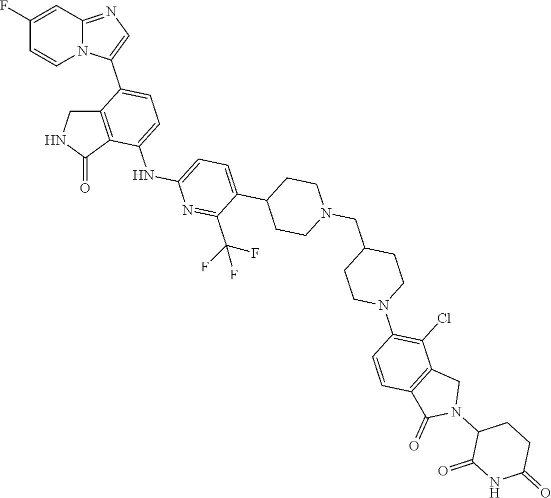 [SMILES: O=C1CCC(N2Cc3c(ccc(N4CCC(CN5CCC(c6ccc(Nc7ccc(-c8cnc9cc(F)ccn89)c8c7C(=O)NC8)nc6C(F)(F)F)CC5)CC4)c3Cl)C2=O)C(=O)N1] |
| 25 | 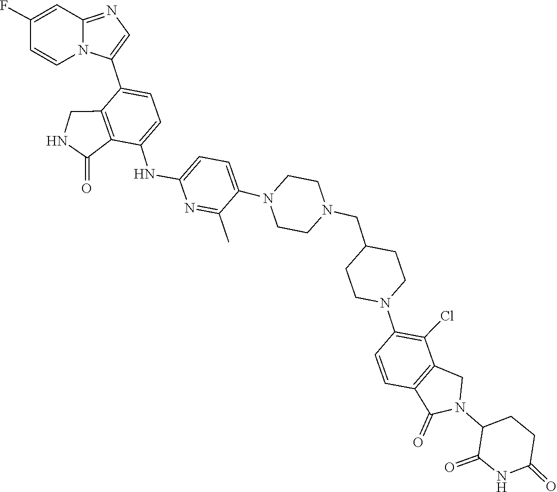 [SMILES: Cc1nc(Nc2ccc(-c3cnc4cc(F)ccn34)c3c2C(=O)NC3)ccc1N1CCN(CC2CCN(c3ccc4c(c3Cl)CN(C3CCC(=O)NC3=O)C4=O)CC2)CC1] |
| 26 | 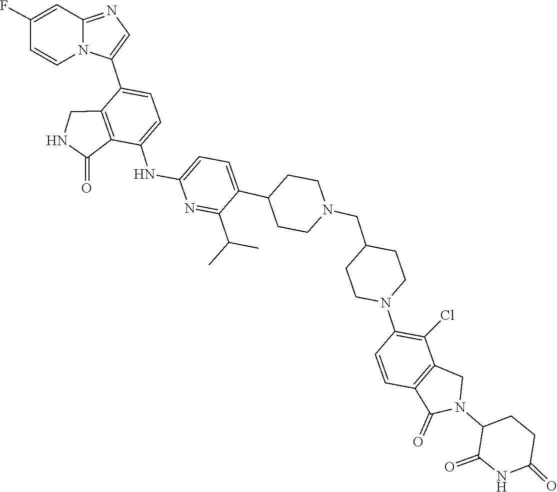 [SMILES: CC(C)c1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 27 | 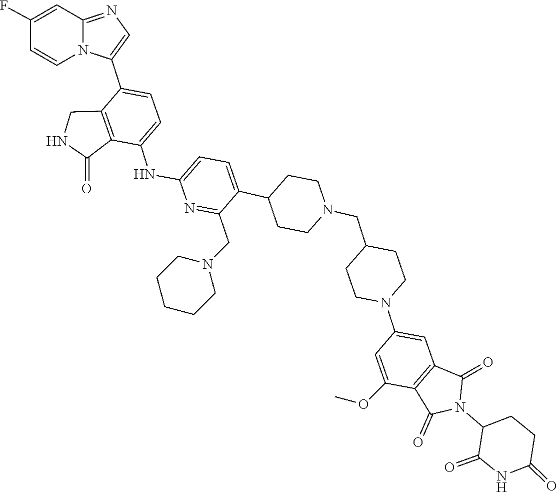 [SMILES: COc1cc(N2CCC(CN3CCC(c4ccc(Nc5ccc(-c6cnc7cc(F)ccn67)c6c5C(=O)NC6)nc4CN4CCCCC4)CC3)CC2)cc2c1C(=O)N(C1CCC(=O)NC1=O)C2=O] |
| 28 | 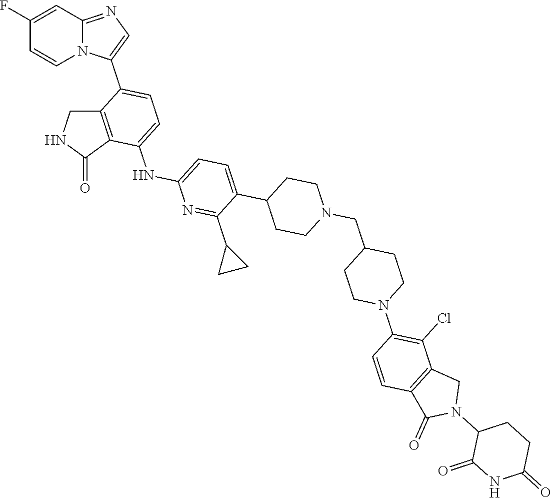 [SMILES: O=C1CCC(N2Cc3c(ccc(N4CCC(CN5CCC(c6ccc(Nc7ccc(-c8cnc9cc(F)ccn89)c8c7C(=O)NC8)nc6C6CC6)CC5)CC4)c3Cl)C2=O)C(=O)N1] |
| 29 | 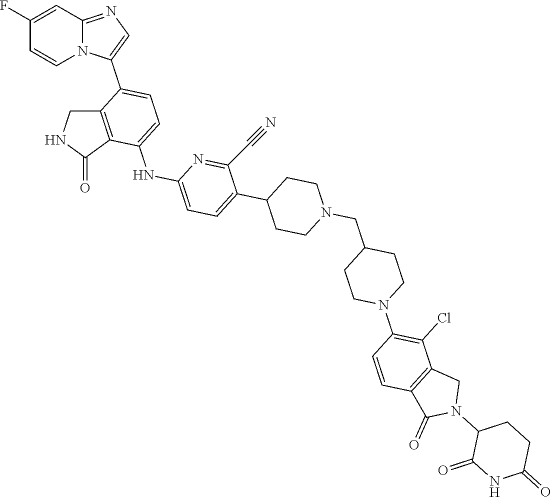 [SMILES: N#Cc1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 30 | 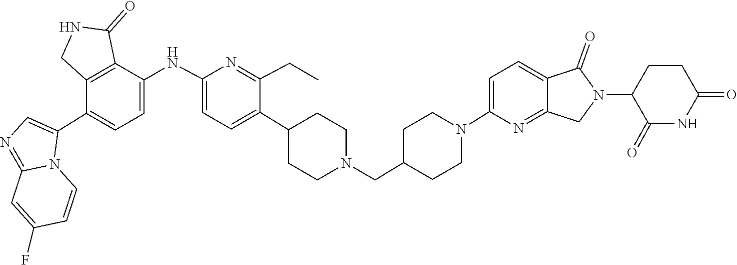 [SMILES: CCc1nc(Nc2ccc(-c3cnc4cc(F)ccn34)c3c2C(=O)NC3)ccc1C1CCN(CC2CCN(c3ccc4c(n3)CN(C3CCC(=O)NC3=O)C4=O)CC2)CC1] |
| 31 | 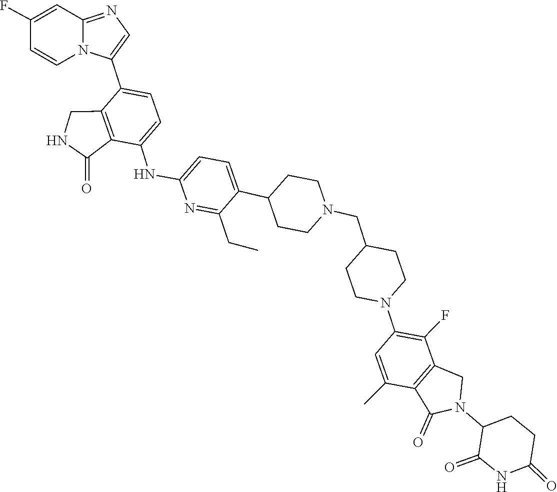 [SMILES: CCc1nc(Nc2ccc(-c3cnc4cc(F)ccn34)c3c2C(=O)NC3)ccc1C1CCN(CC2CCN(c3cc(C)c4c(c3F)CN(C3CCC(=O)NC3=O)C4=O)CC2)CC1] |
| 32 | 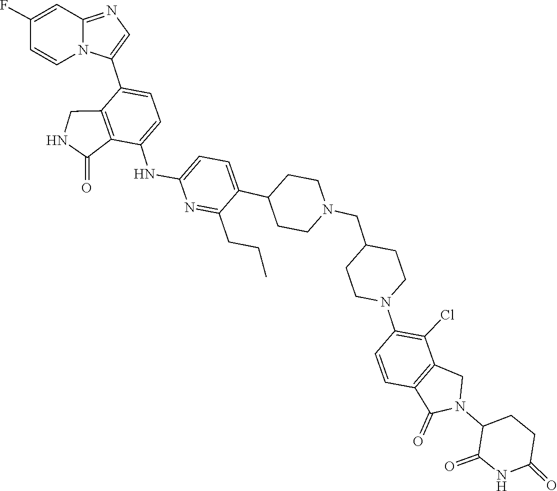 [SMILES: CCCc1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 33 | 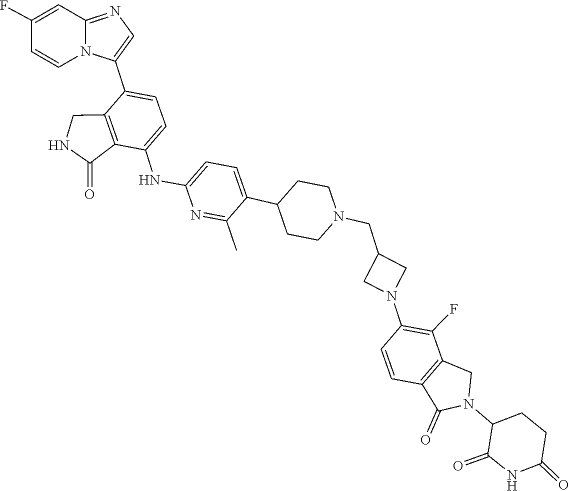 [SMILES: Cc1nc(Nc2ccc(-c3cnc4cc(F)ccn34)c3c2C(=O)NC3)ccc1C1CCN(CC2CN(c3ccc4c(c3F)CN(C3CCC(=O)NC3=O)C4=O)C2)CC1] |
| 34 | 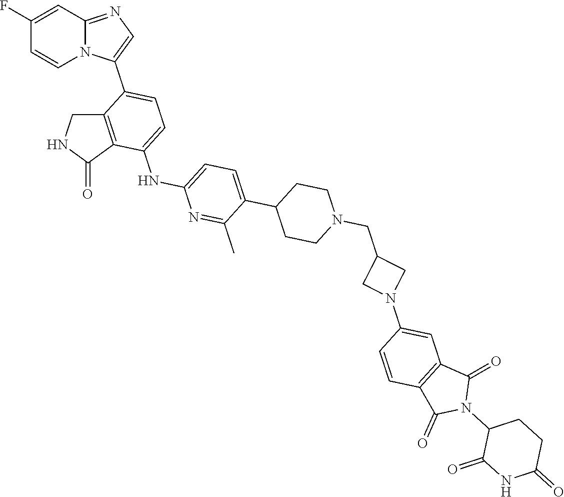 [SMILES: Cc1nc(Nc2ccc(-c3cnc4cc(F)ccn34)c3c2C(=O)NC3)ccc1C1CCN(CC2CN(c3ccc4c(c3)C(=O)N(C3CCC(=O)NC3=O)C4=O)C2)CC1] |
| 35 | 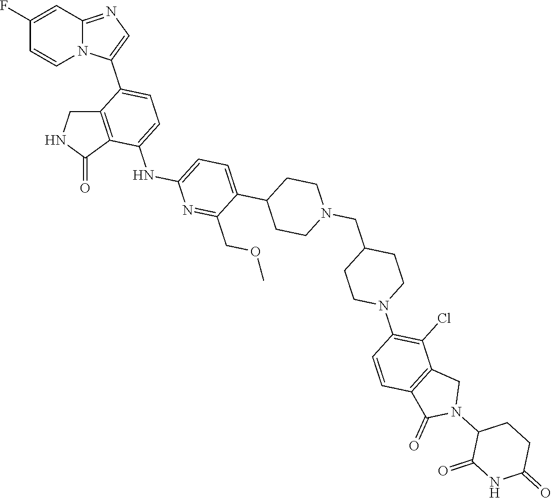 [SMILES: COCc1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 36 | 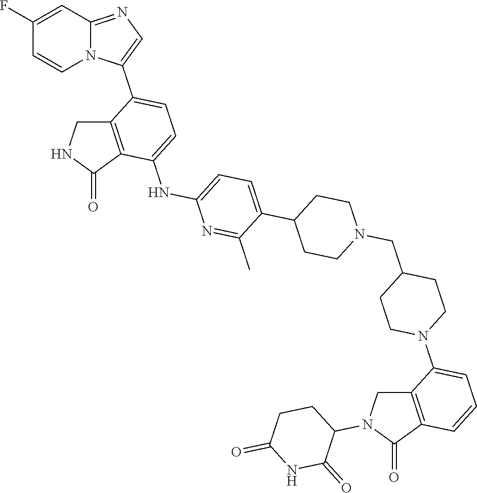 [SMILES: Cc1nc(Nc2ccc(-c3cnc4cc(F)ccn34)c3c2C(=O)NC3)ccc1C1CCN(CC2CCN(c3cccc4c3CN(C3CCC(=O)NC3=O)C4=O)CC2)CC1] |
| 37 | 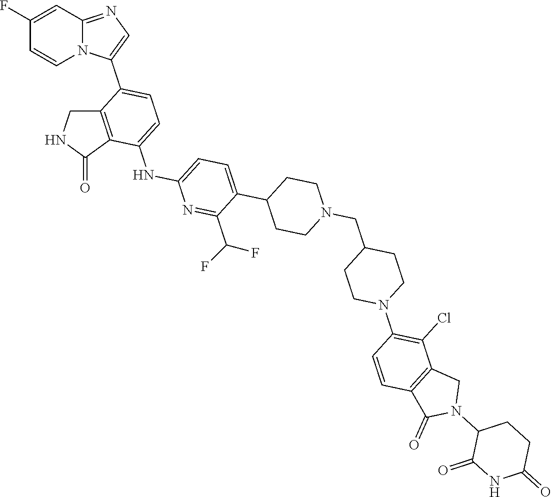 [SMILES: O=C1CCC(N2Cc3c(ccc(N4CCC(CN5CCC(c6ccc(Nc7ccc(-c8cnc9cc(F)ccn89)c8c7C(=O)NC8)nc6C(F)F)CC5)CC4)c3Cl)C2=O)C(=O)N1] |
| 38 | 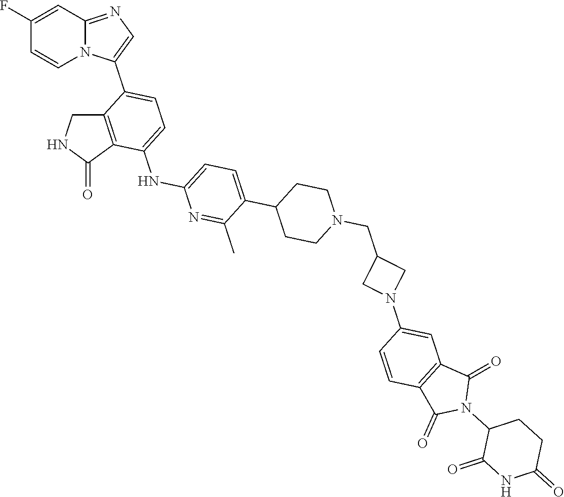 [SMILES: Cc1nc(Nc2ccc(-c3cnc4cc(F)ccn34)c3c2C(=O)NC3)ccc1C1CCN(CC2CN(c3ccc4c(c3)C(=O)N(C3CCC(=O)NC3=O)C4=O)C2)CC1] |
| 39 | 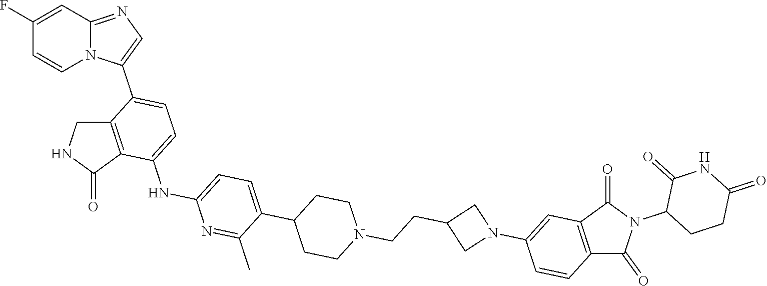 [SMILES: Cc1nc(Nc2ccc(-c3cnc4cc(F)ccn34)c3c2C(=O)NC3)ccc1C1CCN(CCC2CN(c3ccc4c(c3)C(=O)N(C3CCC(=O)NC3=O)C4=O)C2)CC1] |
| 40 | 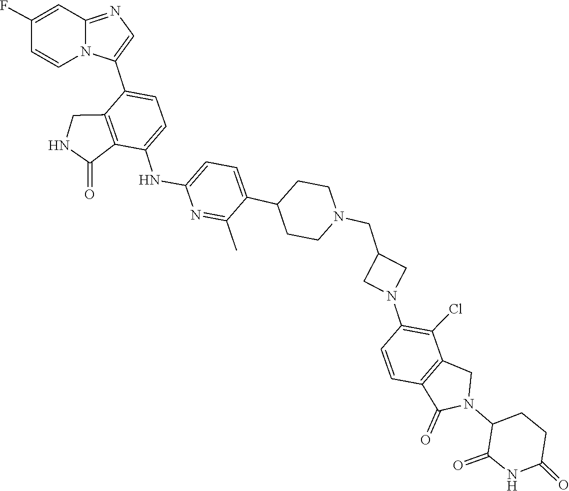 [SMILES: Cc1nc(Nc2ccc(-c3cnc4cc(F)ccn34)c3c2C(=O)NC3)ccc1C1CCN(CC2CN(c3ccc4c(c3Cl)CN(C3CCC(=O)NC3=O)C4=O)C2)CC1] |
| 41 | 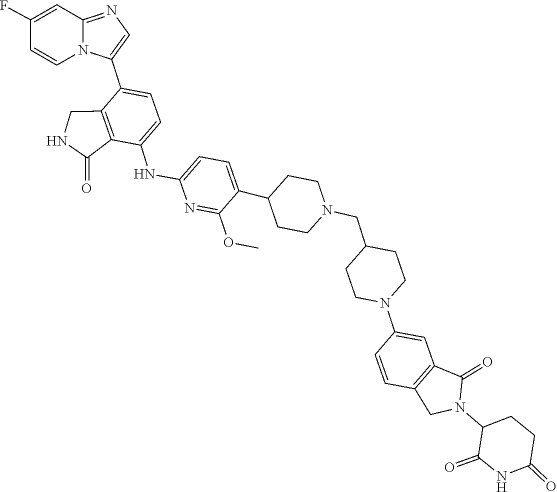 [SMILES: COc1nc(Nc2ccc(-c3cnc4cc(F)ccn34)c3c2C(=O)NC3)ccc1C1CCN(CC2CCN(c3ccc4c(c3)C(=O)N(C3CCC(=O)NC3=O)C4)CC2)CC1] |
| 42 | 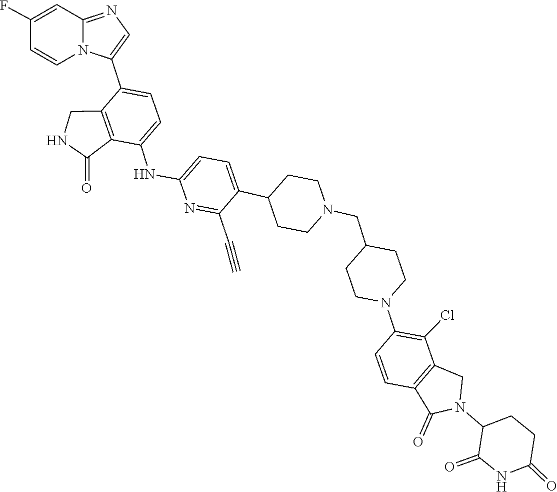 [SMILES: C#Cc1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 43 | 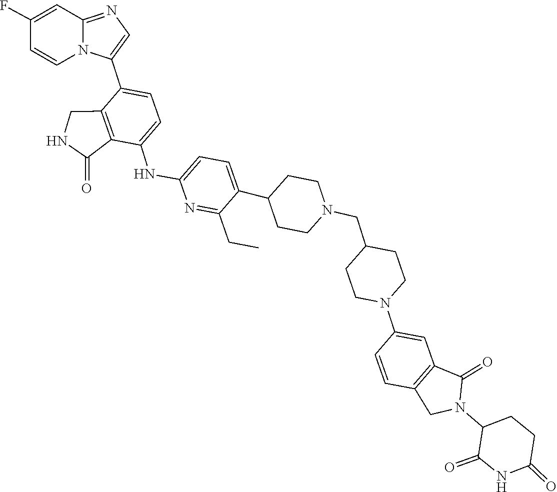 [SMILES: CCc1nc(Nc2ccc(-c3cnc4cc(F)ccn34)c3c2C(=O)NC3)ccc1C1CCN(CC2CCN(c3ccc4c(c3)C(=O)N(C3CCC(=O)NC3=O)C4)CC2)CC1] |
| 44 | 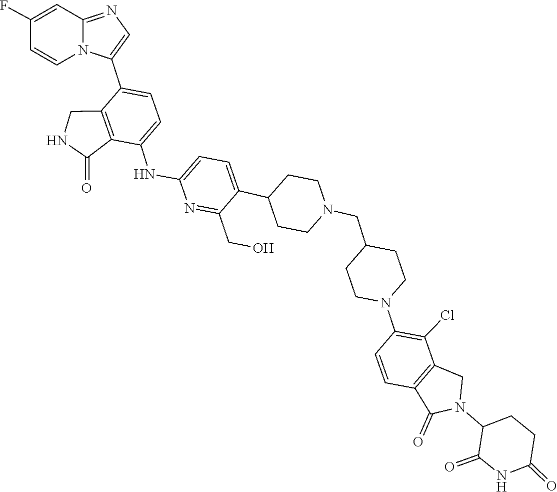 [SMILES: O=C1CCC(N2Cc3c(ccc(N4CCC(CN5CCC(c6ccc(Nc7ccc(-c8cnc9cc(F)ccn89)c8c7C(=O)NC8)nc6CO)CC5)CC4)c3Cl)C2=O)C(=O)N1] |
| 45 | 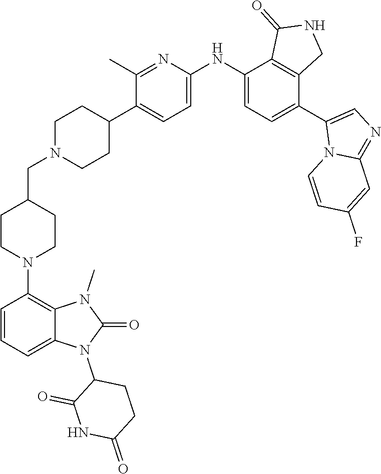 [SMILES: Cc1nc(Nc2ccc(-c3cnc4cc(F)ccn34)c3c2C(=O)NC3)ccc1C1CCN(CC2CCN(c3cccc4c3n(C)c(=O)n4C3CCC(=O)NC3=O)CC2)CC1] |
| 46 | 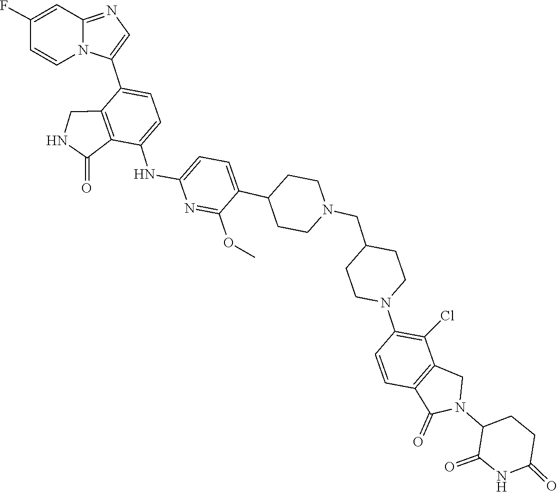 [SMILES: COc1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 47 | 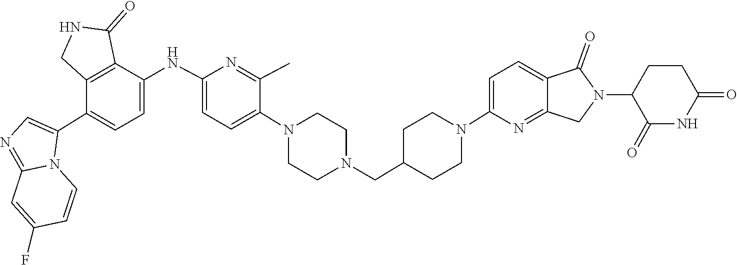 [SMILES: Cc1nc(Nc2ccc(-c3cnc4cc(F)ccn34)c3c2C(=O)NC3)ccc1N1CCN(CC2CCN(c3ccc4c(n3)CN(C3CCC(=O)NC3=O)C4=O)CC2)CC1] |
| 48 | 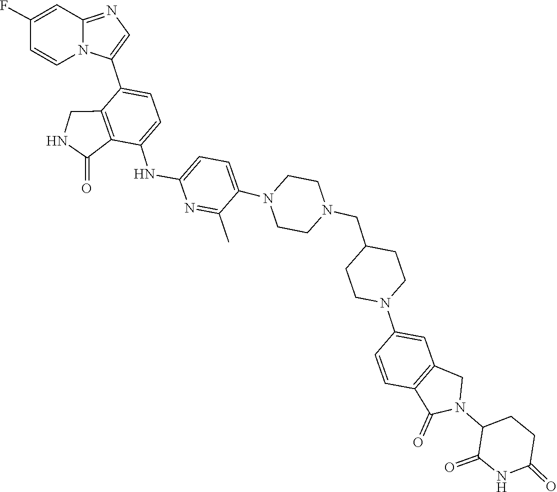 [SMILES: Cc1nc(Nc2ccc(-c3cnc4cc(F)ccn34)c3c2C(=O)NC3)ccc1N1CCN(CC2CCN(c3ccc4c(c3)CN(C3CCC(=O)NC3=O)C4=O)CC2)CC1] |
| 49 | 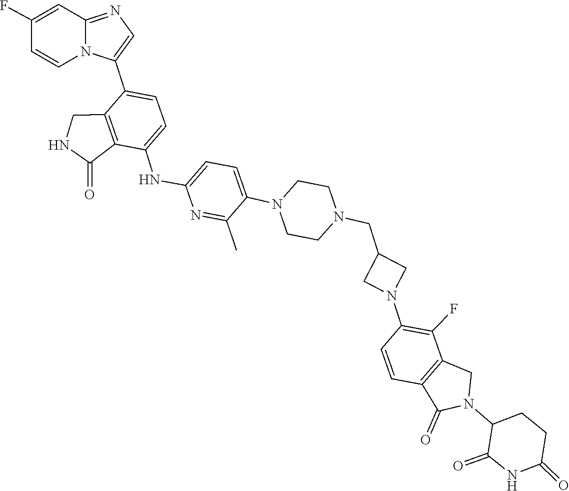 [SMILES: Cc1nc(Nc2ccc(-c3cnc4cc(F)ccn34)c3c2C(=O)NC3)ccc1N1CCN(CC2CN(c3ccc4c(c3F)CN(C3CCC(=O)NC3=O)C4=O)C2)CC1] |
| 50 | 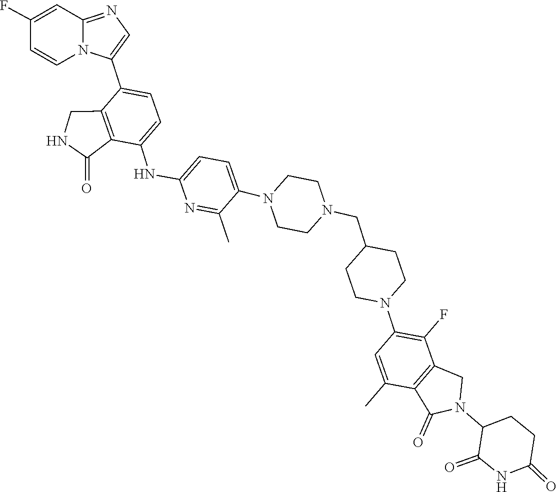 [SMILES: Cc1cc(N2CCC(CN3CCN(c4ccc(Nc5ccc(-c6cnc7cc(F)ccn67)c6c5C(=O)NC6)nc4C)CC3)CC2)c(F)c2c1C(=O)N(C1CCC(=O)NC1=O)C2] |
| 51 | 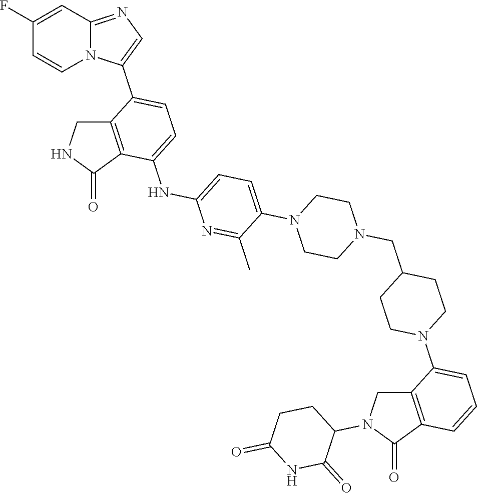 [SMILES: Cc1nc(Nc2ccc(-c3cnc4cc(F)ccn34)c3c2C(=O)NC3)ccc1N1CCN(CC2CCN(c3cccc4c3CN(C3CCC(=O)NC3=O)C4=O)CC2)CC1] |
| 52 | 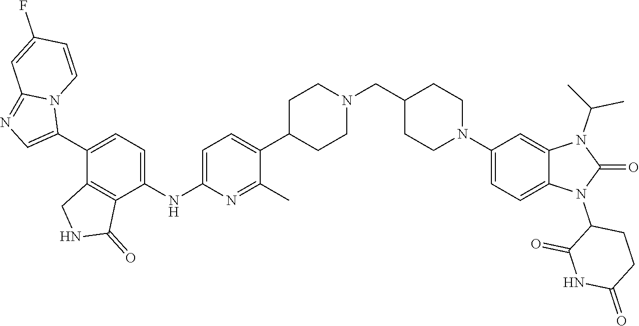 [SMILES: Cc1nc(Nc2ccc(-c3cnc4cc(F)ccn34)c3c2C(=O)NC3)ccc1C1CCN(CC2CCN(c3ccc4c(c3)n(C(C)C)c(=O)n4C3CCC(=O)NC3=O)CC2)CC1] |
| 53 | 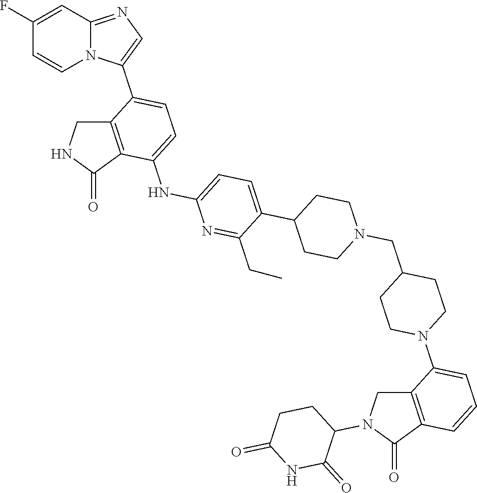 [SMILES: CCc1nc(Nc2ccc(-c3cnc4cc(F)ccn34)c3c2C(=O)NC3)ccc1C1CCN(CC2CCN(c3cccc4c3CN(C3CCC(=O)NC3=O)C4=O)CC2)CC1] |
| 54 | 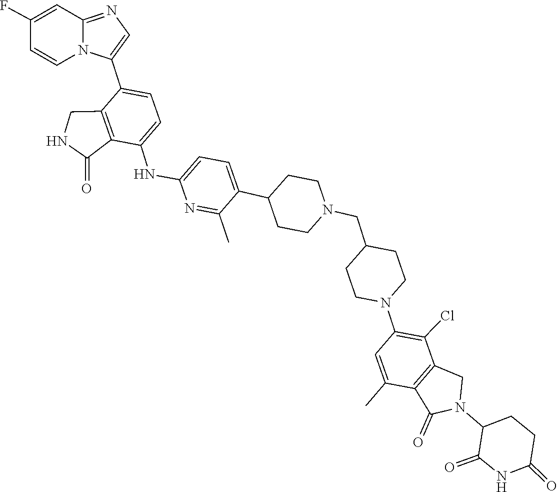 [SMILES: Cc1cc(N2CCC(CN3CCC(c4ccc(Nc5ccc(-c6cnc7cc(F)ccn67)c6c5C(=O)NC6)nc4C)CC3)CC2)c(Cl)c2c1C(=O)N(C1CCC(=O)NC1=O)C2] |
| 55 | 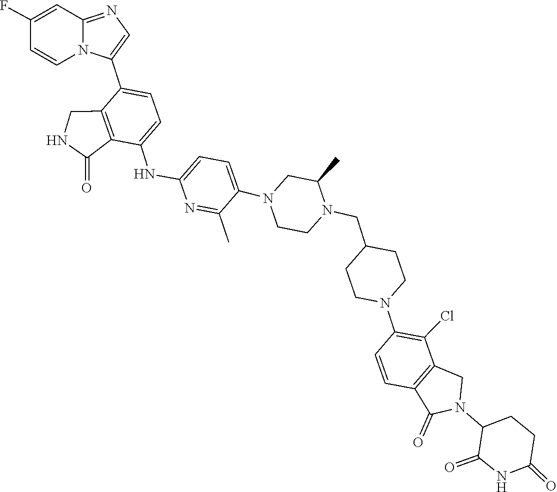 [SMILES: Cc1nc(Nc2ccc(-c3cnc4cc(F)ccn34)c3c2C(=O)NC3)ccc1N1CCN(CC2CCN(c3ccc4c(c3Cl)CN(C3CCC(=O)NC3=O)C4=O)CC2)[C@H](C)C1] |
| 56 | 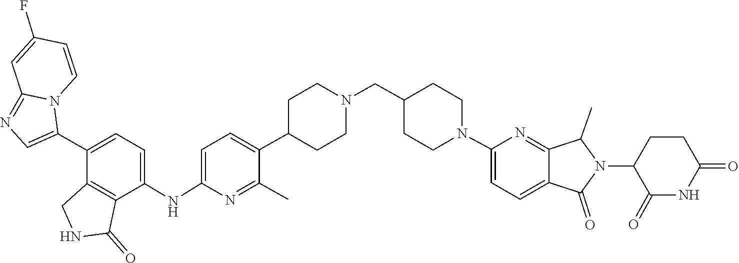 [SMILES: Cc1nc(Nc2ccc(-c3cnc4cc(F)ccn34)c3c2C(=O)NC3)ccc1C1CCN(CC2CCN(c3ccc4c(n3)C(C)N(C3CCC(=O)NC3=O)C4=O)CC2)CC1] |
| 57 | 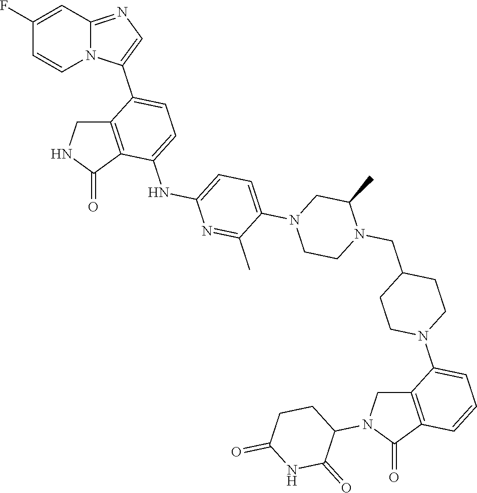 [SMILES: Cc1nc(Nc2ccc(-c3cnc4cc(F)ccn34)c3c2C(=O)NC3)ccc1N1CCN(CC2CCN(c3cccc4c3CN(C3CCC(=O)NC3=O)C4=O)CC2)[C@H](C)C1] |
| 58 | 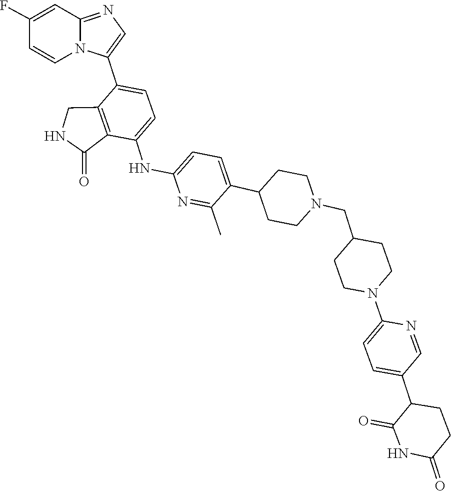 [SMILES: Cc1nc(Nc2ccc(-c3cnc4cc(F)ccn34)c3c2C(=O)NC3)ccc1C1CCN(CC2CCN(c3ccc(C4CCC(=O)NC4=O)cn3)CC2)CC1] |
| 59 | 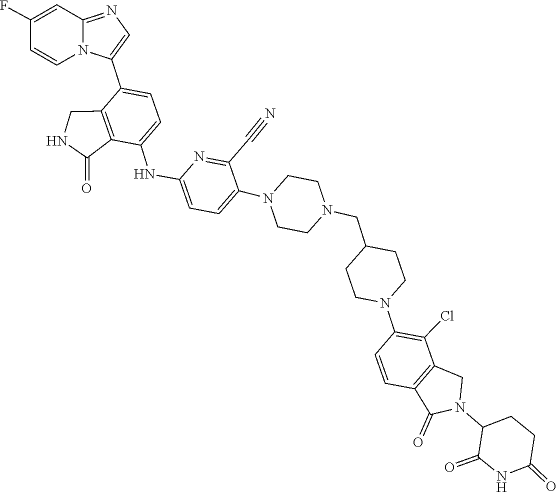 [SMILES: N#Cc1nc(Nc2ccc(-c3cnc4cc(F)ccn34)c3c2C(=O)NC3)ccc1N1CCN(CC2CCN(c3ccc4c(c3Cl)CN(C3CCC(=O)NC3=O)C4=O)CC2)CC1] |
| 60 | 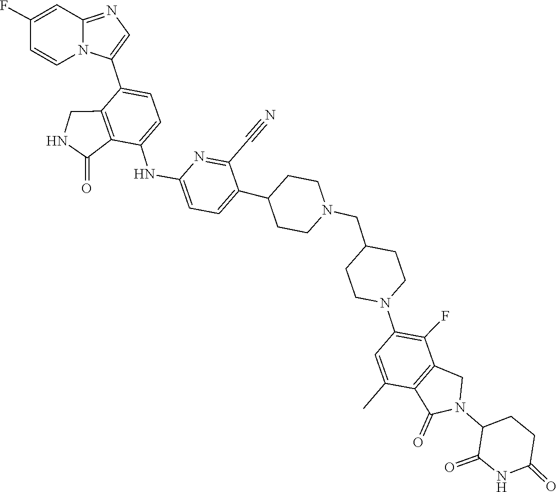 [SMILES: Cc1cc(N2CCC(CN3CCC(c4ccc(Nc5ccc(-c6cnc7cc(F)ccn67)c6c5C(=O)NC6)nc4C#N)CC3)CC2)c(F)c2c1C(=O)N(C1CCC(=O)NC1=O)C2] |
| 61 | 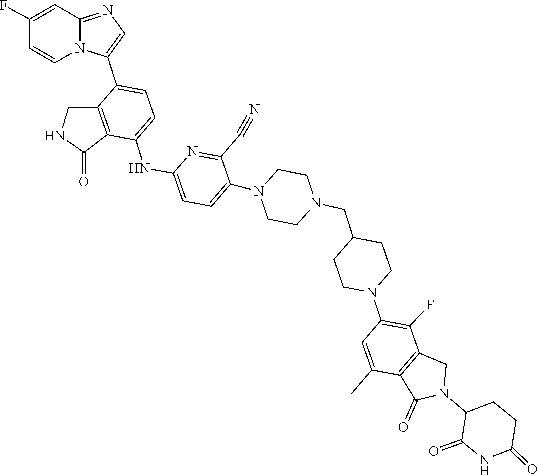 [SMILES: Cc1cc(N2CCC(CN3CCN(c4ccc(Nc5ccc(-c6cnc7cc(F)ccn67)c6c5C(=O)NC6)nc4C#N)CC3)CC2)c(F)c2c1C(=O)N(C1CCC(=O)NC1=O)C2] |
| 62 | 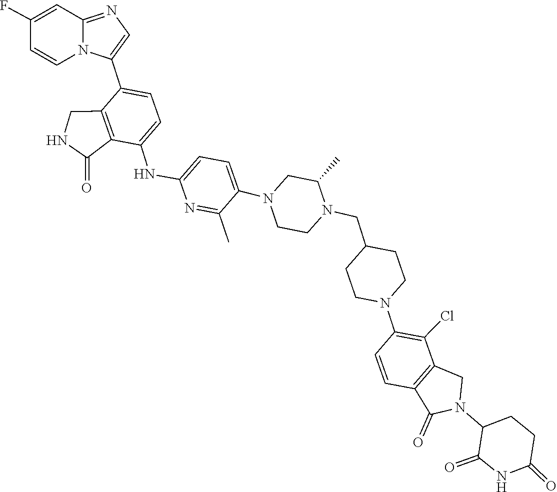 [SMILES: Cc1nc(Nc2ccc(-c3cnc4cc(F)ccn34)c3c2C(=O)NC3)ccc1N1CCN(CC2CCN(c3ccc4c(c3Cl)CN(C3CCC(=O)NC3=O)C4=O)CC2)[C@@H](C)C1] |
| 63 | 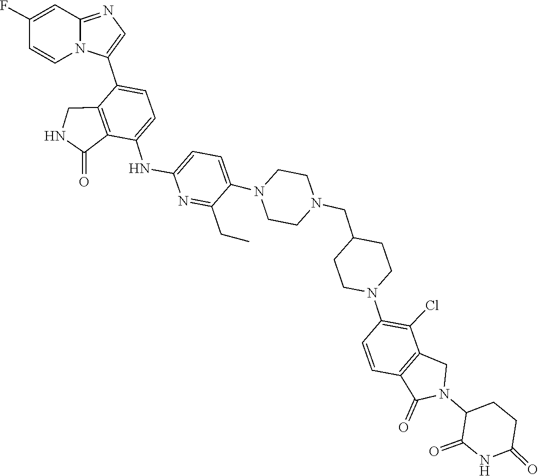 [SMILES: CCc1nc(Nc2ccc(-c3cnc4cc(F)ccn34)c3c2C(=O)NC3)ccc1N1CCN(CC2CCN(c3ccc4c(c3Cl)CN(C3CCC(=O)NC3=O)C4=O)CC2)CC1] |
| 64 | 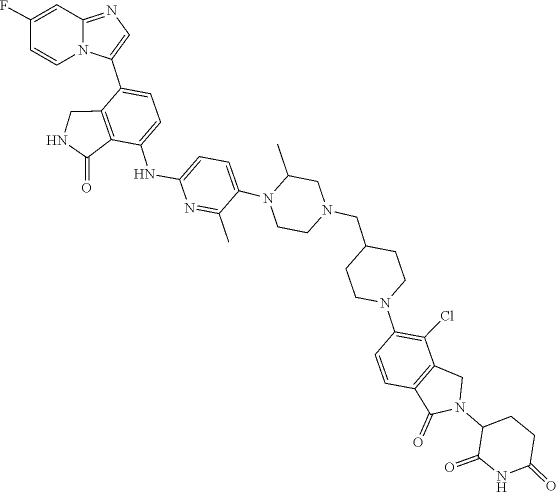 [SMILES: Cc1nc(Nc2ccc(-c3cnc4cc(F)ccn34)c3c2C(=O)NC3)ccc1N1CCN(CC2CCN(c3ccc4c(c3Cl)CN(C3CCC(=O)NC3=O)C4=O)CC2)CC1C] |
| 65 | 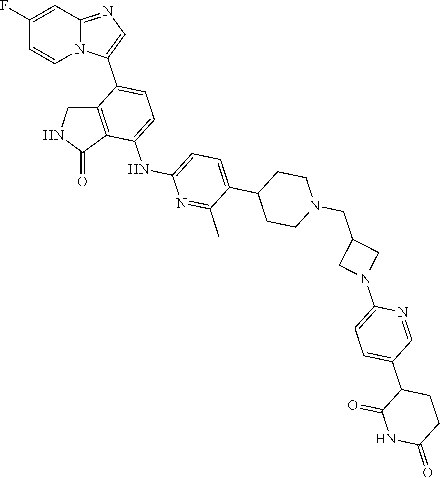 [SMILES: Cc1nc(Nc2ccc(-c3cnc4cc(F)ccn34)c3c2C(=O)NC3)ccc1C1CCN(CC2CN(c3ccc(C4CCC(=O)NC4=O)cn3)C2)CC1] |
| 66 | 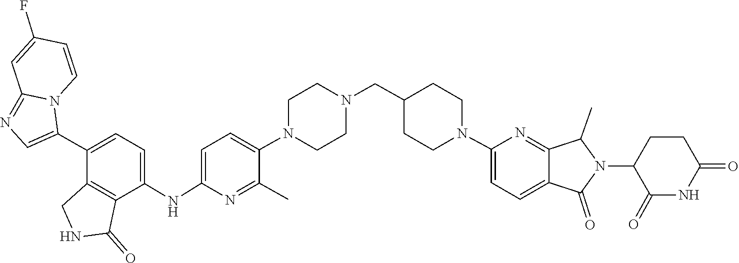 [SMILES: Cc1nc(Nc2ccc(-c3cnc4cc(F)ccn34)c3c2C(=O)NC3)ccc1N1CCN(CC2CCN(c3ccc4c(n3)C(C)N(C3CCC(=O)NC3=O)C4=O)CC2)CC1] |
| 67 | 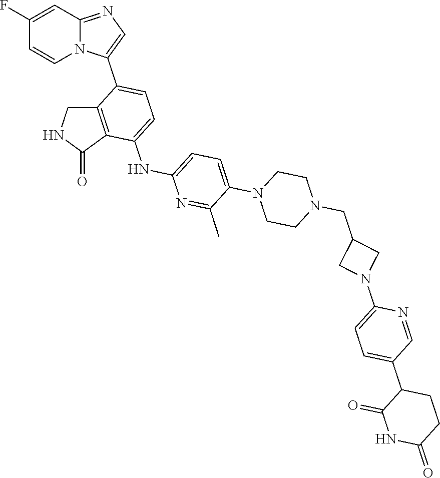 [SMILES: Cc1nc(Nc2ccc(-c3cnc4cc(F)ccn34)c3c2C(=O)NC3)ccc1N1CCN(CC2CN(c3ccc(C4CCC(=O)NC4=O)cn3)C2)CC1] |
| 68 | 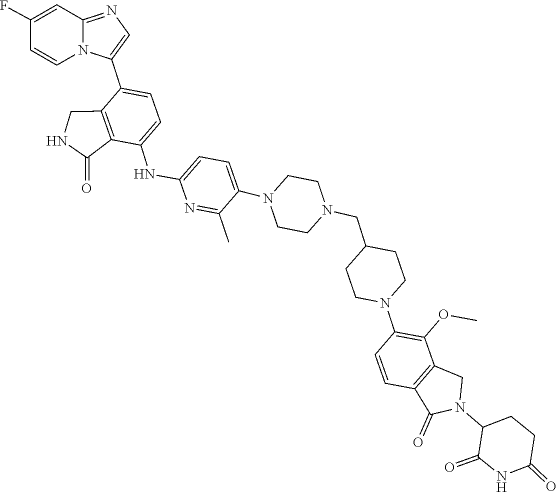 [SMILES: COc1c(N2CCC(CN3CCN(c4ccc(Nc5ccc(-c6cnc7cc(F)ccn67)c6c5C(=O)NC6)nc4C)CC3)CC2)ccc2c1CN(C1CCC(=O)NC1=O)C2=O] |
| 69 | 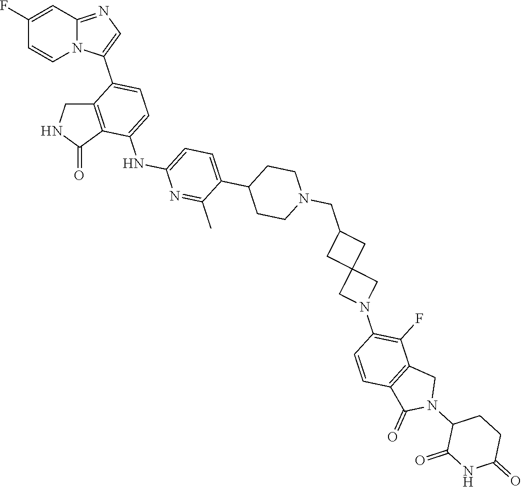 [SMILES: Cc1nc(Nc2ccc(-c3cnc4cc(F)ccn34)c3c2C(=O)NC3)ccc1C1CCN(CC2CC3(C2)CN(c2ccc4c(c2F)CN(C2CCC(=O)NC2=O)C4=O)C3)CC1] |
| 70 | 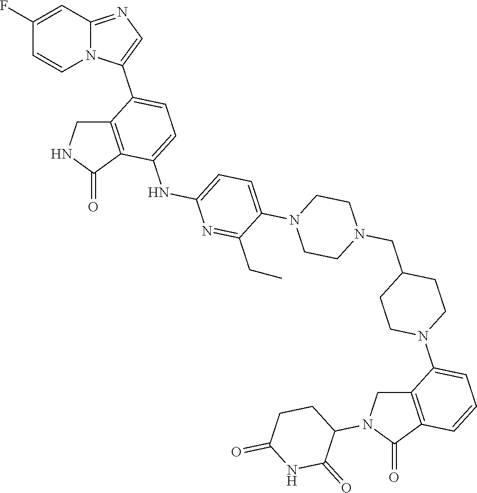 [SMILES: CCc1nc(Nc2ccc(-c3cnc4cc(F)ccn34)c3c2C(=O)NC3)ccc1N1CCN(CC2CCN(c3cccc4c3CN(C3CCC(=O)NC3=O)C4=O)CC2)CC1] |
| 71 | 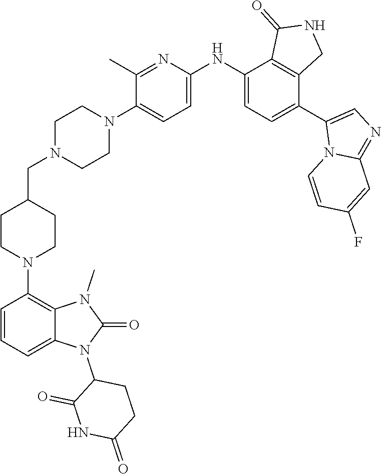 [SMILES: Cc1nc(Nc2ccc(-c3cnc4cc(F)ccn34)c3c2C(=O)NC3)ccc1N1CCN(CC2CCN(c3cccc4c3n(C)c(=O)n4C3CCC(=O)NC3=O)CC2)CC1] |
| 72 | 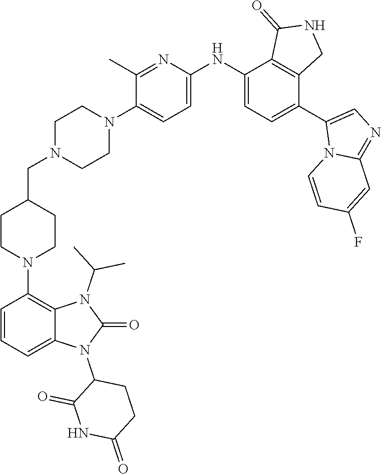 [SMILES: Cc1nc(Nc2ccc(-c3cnc4cc(F)ccn34)c3c2C(=O)NC3)ccc1N1CCN(CC2CCN(c3cccc4c3n(C(C)C)c(=O)n4C3CCC(=O)NC3=O)CC2)CC1] |
| 73 | 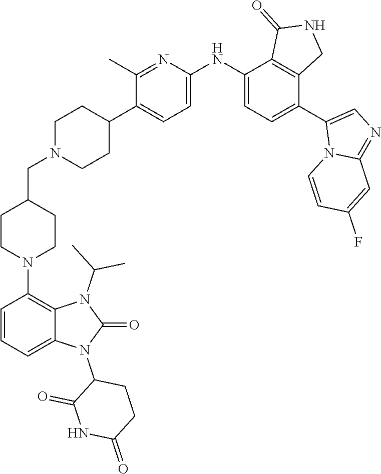 [SMILES: Cc1nc(Nc2ccc(-c3cnc4cc(F)ccn34)c3c2C(=O)NC3)ccc1C1CCN(CC2CCN(c3cccc4c3n(C(C)C)c(=O)n4C3CCC(=O)NC3=O)CC2)CC1] |
| 74 | 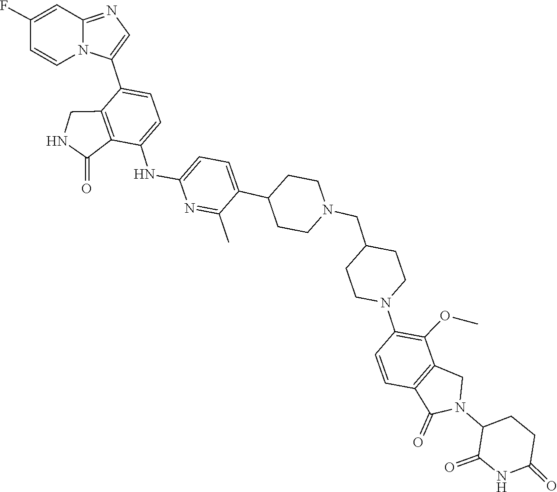 [SMILES: COc1c(N2CCC(CN3CCC(c4ccc(Nc5ccc(-c6cnc7cc(F)ccn67)c6c5C(=O)NC6)nc4C)CC3)CC2)ccc2c1CN(C1CCC(=O)NC1=O)C2=O] |
| 75 | 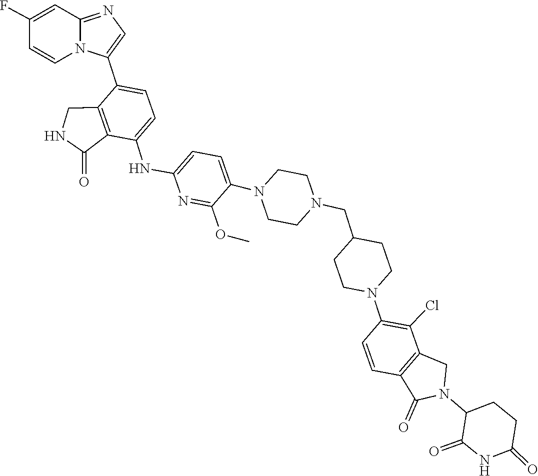 [SMILES: COc1nc(Nc2ccc(-c3cnc4cc(F)ccn34)c3c2C(=O)NC3)ccc1N1CCN(CC2CCN(c3ccc4c(c3Cl)CN(C3CCC(=O)NC3=O)C4=O)CC2)CC1] |
| 76 | 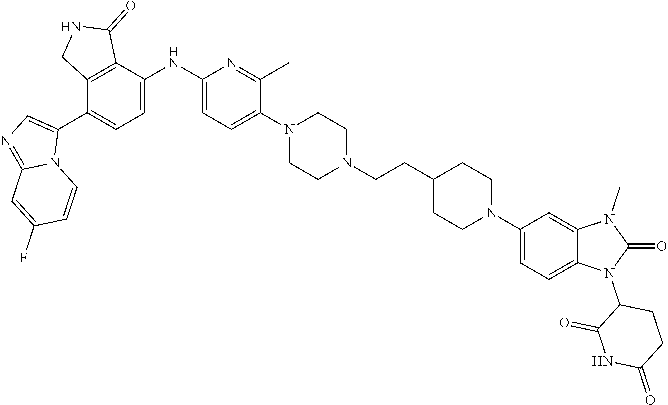 [SMILES: Cc1nc(Nc2ccc(-c3cnc4cc(F)ccn34)c3c2C(=O)NC3)ccc1N1CCN(CCC2CCN(c3ccc4c(c3)n(C)c(=O)n4C3CCC(=O)NC3=O)CC2)CC1] |
| 77 | 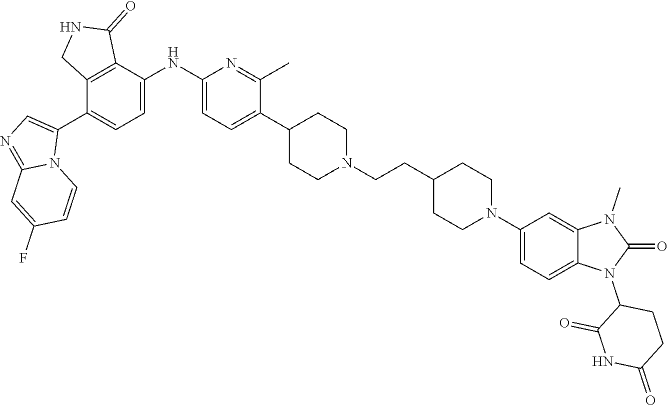 [SMILES: Cc1nc(Nc2ccc(-c3cnc4cc(F)ccn34)c3c2C(=O)NC3)ccc1C1CCN(CCC2CCN(c3ccc4c(c3)n(C)c(=O)n4C3CCC(=O)NC3=O)CC2)CC1] |
| 78 | 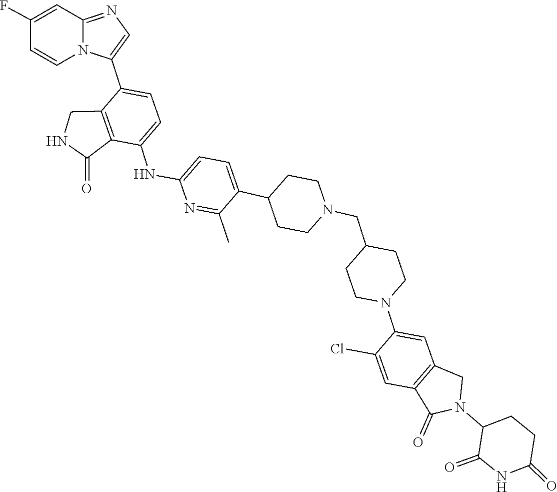 [SMILES: Cc1nc(Nc2ccc(-c3cnc4cc(F)ccn34)c3c2C(=O)NC3)ccc1C1CCN(CC2CCN(c3cc4c(cc3Cl)C(=O)N(C3CCC(=O)NC3=O)C4)CC2)CC1] |
| 79 | 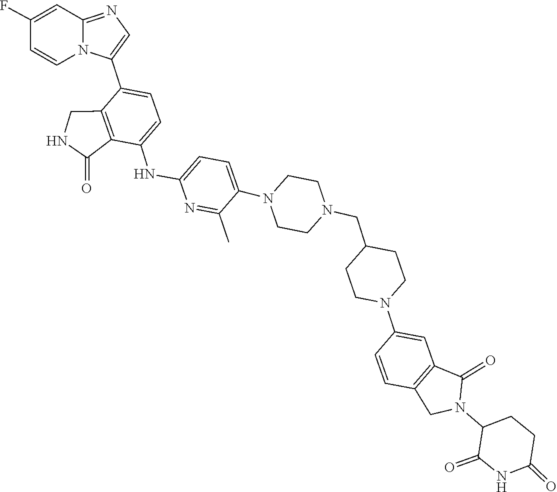 [SMILES: Cc1nc(Nc2ccc(-c3cnc4cc(F)ccn34)c3c2C(=O)NC3)ccc1N1CCN(CC2CCN(c3ccc4c(c3)C(=O)N(C3CCC(=O)NC3=O)C4)CC2)CC1] |
| 80 | 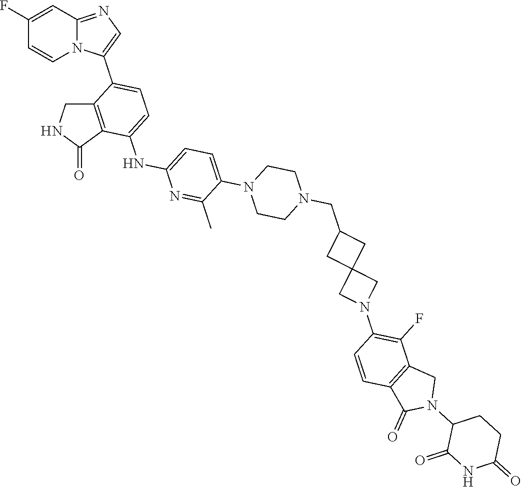 [SMILES: Cc1nc(Nc2ccc(-c3cnc4cc(F)ccn34)c3c2C(=O)NC3)ccc1N1CCN(CC2CC3(C2)CN(c2ccc4c(c2F)CN(C2CCC(=O)NC2=O)C4=O)C3)CC1] |
| 81 | 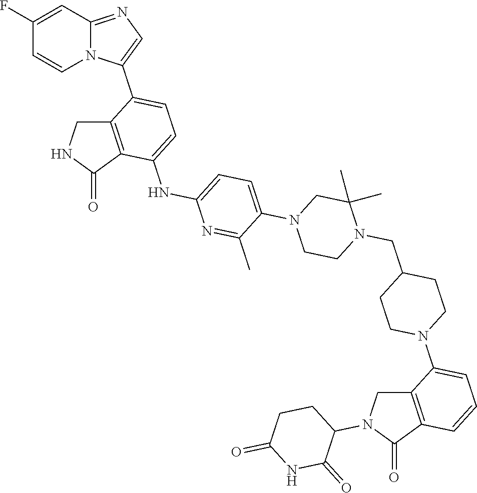 [SMILES: Cc1nc(Nc2ccc(-c3cnc4cc(F)ccn34)c3c2C(=O)NC3)ccc1N1CCN(CC2CCN(c3cccc4c3CN(C3CCC(=O)NC3=O)C4=O)CC2)C(C)(C)C1] |
| 82 | 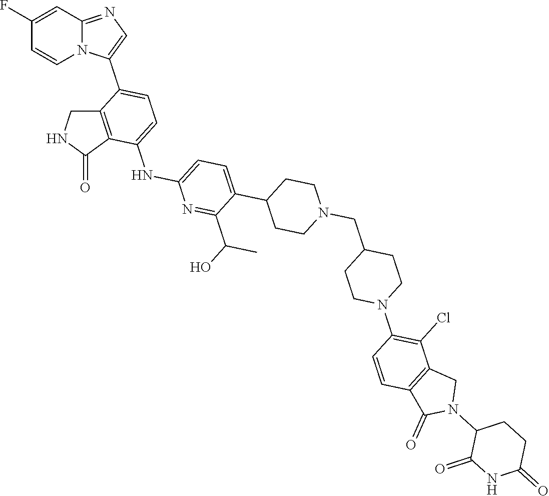 [SMILES: CC(O)c1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 83 | 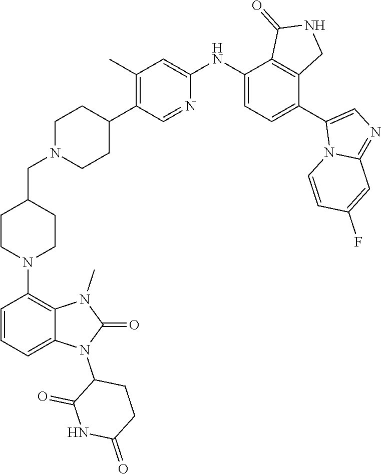 [SMILES: Cc1cc(Nc2ccc(-c3cnc4cc(F)ccn34)c3c2C(=O)NC3)ncc1C1CCN(CC2CCN(c3cccc4c3n(C)c(=O)n4C3CCC(=O)NC3=O)CC2)CC1] |
| 84 | 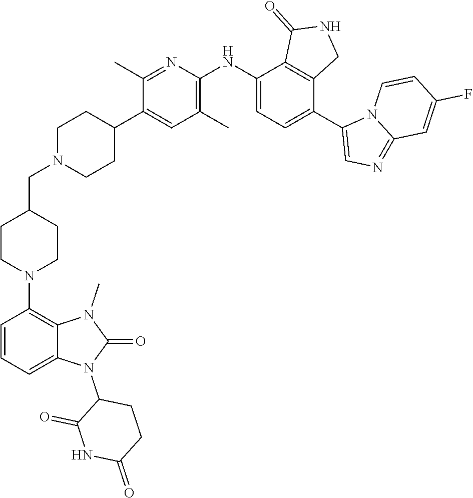 [SMILES: Cc1cc(C2CCN(CC3CCN(c4cccc5c4n(C)c(=O)n5C4CCC(=O)NC4=O)CC3)CC2)c(C)nc1Nc1ccc(-c2cnc3cc(F)ccn23)c2c1C(=O)NC2] |
| 85 | 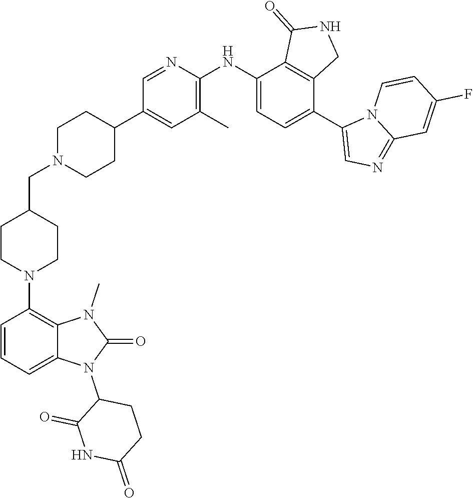 [SMILES: Cc1cc(C2CCN(CC3CCN(c4cccc5c4n(C)c(=O)n5C4CCC(=O)NC4=O)CC3)CC2)cnc1Nc1ccc(-c2cnc3cc(F)ccn23)c2c1C(=O)NC2] |
| 86 | 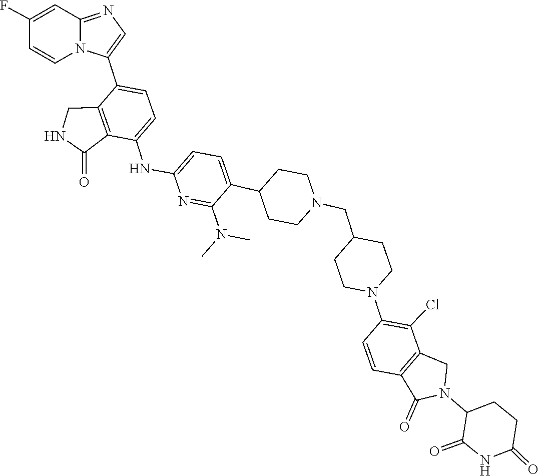 [SMILES: CN(C)c1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 87 | 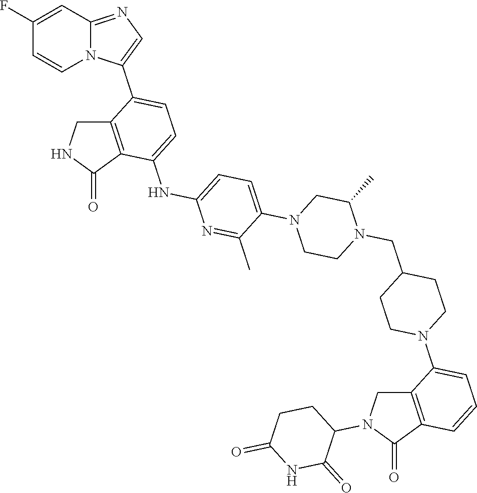 [SMILES: Cc1nc(Nc2ccc(-c3cnc4cc(F)ccn34)c3c2C(=O)NC3)ccc1N1CCN(CC2CCN(c3cccc4c3CN(C3CCC(=O)NC3=O)C4=O)CC2)[C@@H](C)C1] |
| 88 | 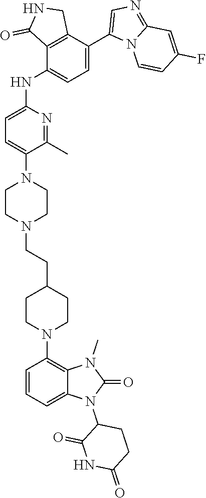 [SMILES: Cc1nc(Nc2ccc(-c3cnc4cc(F)ccn34)c3c2C(=O)NC3)ccc1N1CCN(CCC2CCN(c3cccc4c3n(C)c(=O)n4C3CCC(=O)NC3=O)CC2)CC1] |
| 89 | 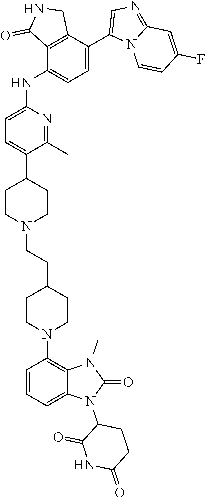 [SMILES: Cc1nc(Nc2ccc(-c3cnc4cc(F)ccn34)c3c2C(=O)NC3)ccc1C1CCN(CCC2CCN(c3cccc4c3n(C)c(=O)n4C3CCC(=O)NC3=O)CC2)CC1] |
| 90 | 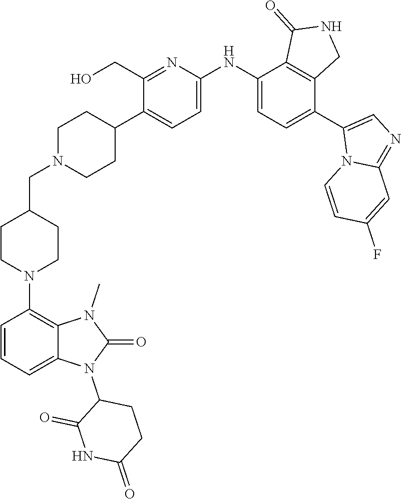 [SMILES: Cn1c(=O)n(C2CCC(=O)NC2=O)c2cccc(N3CCC(CN4CCC(c5ccc(Nc6ccc(-c7cnc8cc(F)ccn78)c7c6C(=O)NC7)nc5CO)CC4)CC3)c21] |
| 91 | 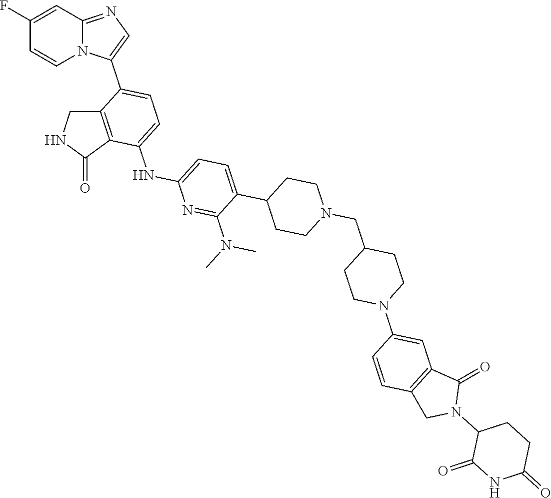 [SMILES: CN(C)c1nc(Nc2ccc(-c3cnc4cc(F)ccn34)c3c2C(=O)NC3)ccc1C1CCN(CC2CCN(c3ccc4c(c3)C(=O)N(C3CCC(=O)NC3=O)C4)CC2)CC1] |
| 92 | 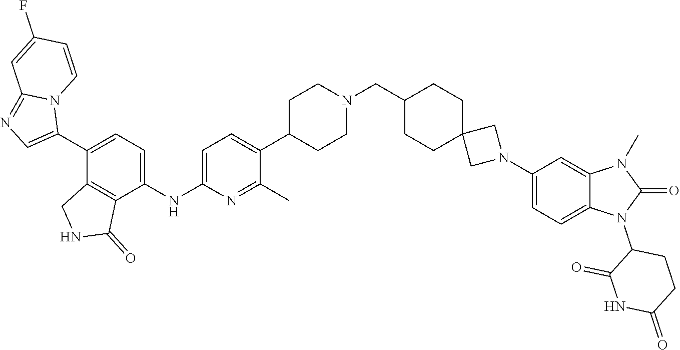 [SMILES: Cc1nc(Nc2ccc(-c3cnc4cc(F)ccn34)c3c2C(=O)NC3)ccc1C1CCN(CC2CCC3(CC2)CN(c2ccc4c(c2)n(C)c(=O)n4C2CCC(=O)NC2=O)C3)CC1] |
| 93 | 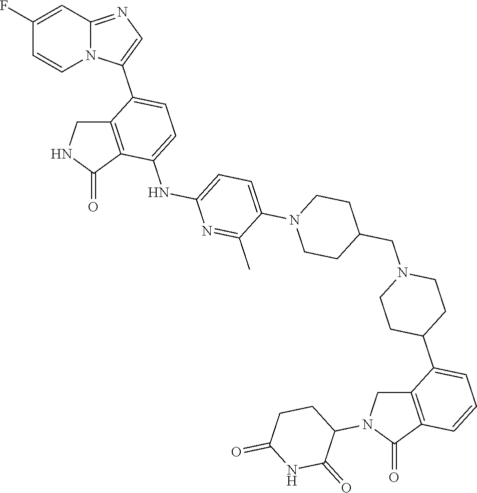 [SMILES: Cc1nc(Nc2ccc(-c3cnc4cc(F)ccn34)c3c2C(=O)NC3)ccc1N1CCC(CN2CCC(c3cccc4c3CN(C3CCC(=O)NC3=O)C4=O)CC2)CC1] |
| 94 | 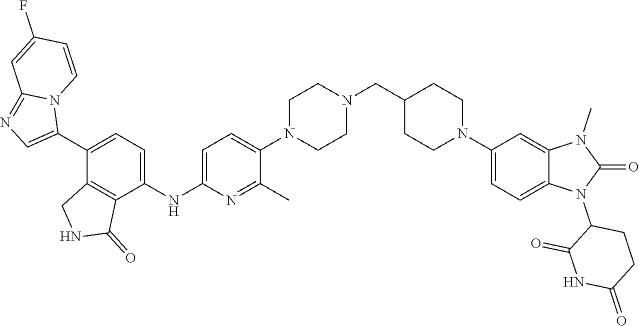 [SMILES: Cc1nc(Nc2ccc(-c3cnc4cc(F)ccn34)c3c2C(=O)NC3)ccc1N1CCN(CC2CCN(c3ccc4c(c3)n(C)c(=O)n4C3CCC(=O)NC3=O)CC2)CC1] |
| 95 | 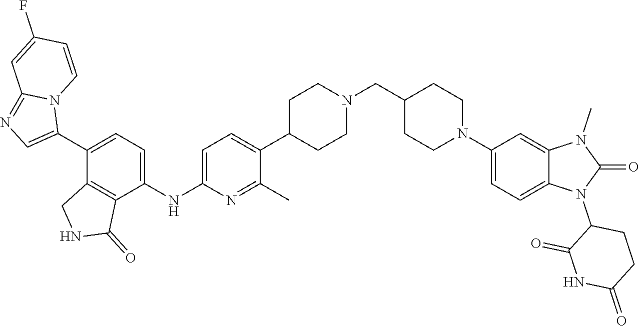 [SMILES: Cc1nc(Nc2ccc(-c3cnc4cc(F)ccn34)c3c2C(=O)NC3)ccc1C1CCN(CC2CCN(c3ccc4c(c3)n(C)c(=O)n4C3CCC(=O)NC3=O)CC2)CC1] |
| 96 | 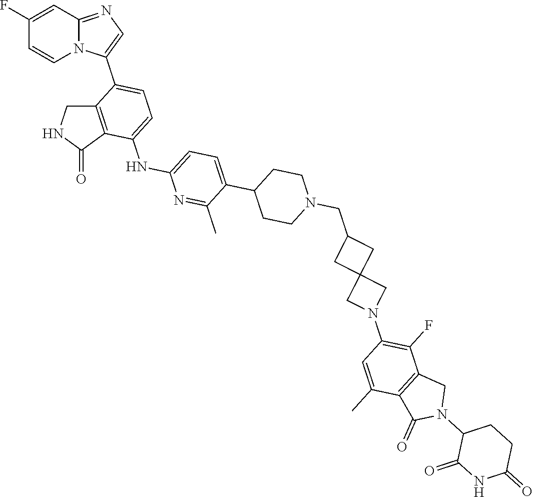 [SMILES: Cc1cc(N2CC3(CC(CN4CCC(c5ccc(Nc6ccc(-c7cnc8cc(F)ccn78)c7c6C(=O)NC7)nc5C)CC4)C3)C2)c(F)c2c1C(=O)N(C1CCC(=O)NC1=O)C2] |
| 97 | 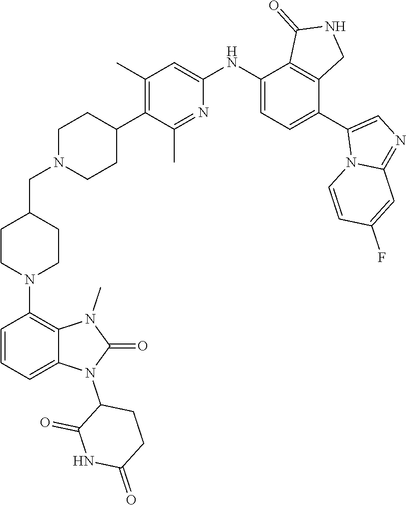 [SMILES: Cc1cc(Nc2ccc(-c3cnc4cc(F)ccn34)c3c2C(=O)NC3)nc(C)c1C1CCN(CC2CCN(c3cccc4c3n(C)c(=O)n4C3CCC(=O)NC3=O)CC2)CC1] |
| 98 | 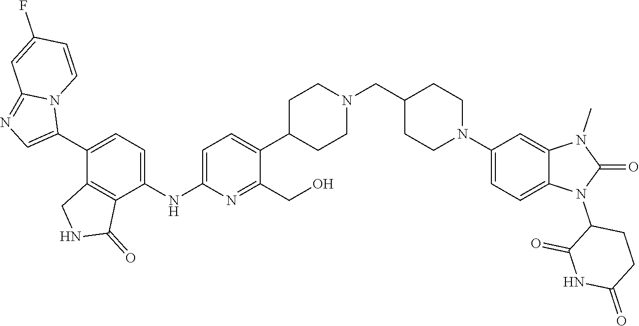 [SMILES: Cn1c(=O)n(C2CCC(=O)NC2=O)c2ccc(N3CCC(CN4CCC(c5ccc(Nc6ccc(-c7cnc8cc(F)ccn78)c7c6C(=O)NC7)nc5CO)CC4)CC3)cc21] |
| 99 | 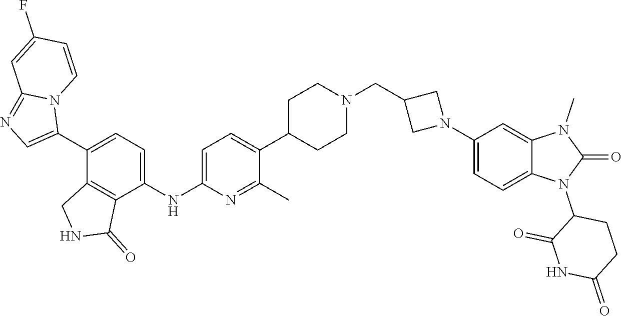 [SMILES: Cc1nc(Nc2ccc(-c3cnc4cc(F)ccn34)c3c2C(=O)NC3)ccc1C1CCN(CC2CN(c3ccc4c(c3)n(C)c(=O)n4C3CCC(=O)NC3=O)C2)CC1] |
| 100 | 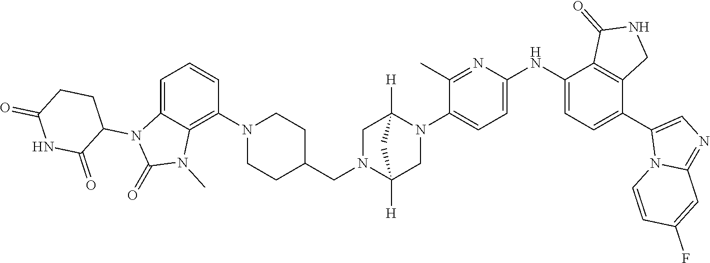 [SMILES: Cc1nc(Nc2ccc(-c3cnc4cc(F)ccn34)c3c2C(=O)NC3)ccc1N1C[C@@H]2C[C@H]1CN2CC1CCN(c2cccc3c2n(C)c(=O)n3C2CCC(=O)NC2=O)CC1] |
| 101 | 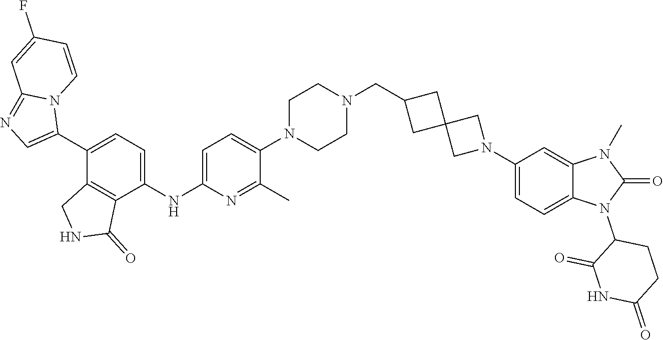 [SMILES: Cc1nc(Nc2ccc(-c3cnc4cc(F)ccn34)c3c2C(=O)NC3)ccc1N1CCN(CC2CC3(C2)CN(c2ccc4c(c2)n(C)c(=O)n4C2CCC(=O)NC2=O)C3)CC1] |
| 102 | 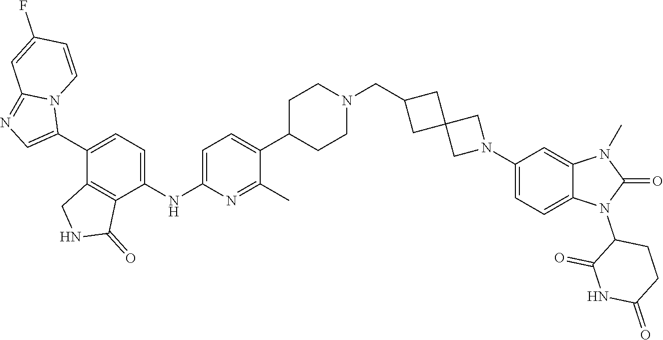 [SMILES: Cc1nc(Nc2ccc(-c3cnc4cc(F)ccn34)c3c2C(=O)NC3)ccc1C1CCN(CC2CC3(C2)CN(c2ccc4c(c2)n(C)c(=O)n4C2CCC(=O)NC2=O)C3)CC1] |
| 103 | 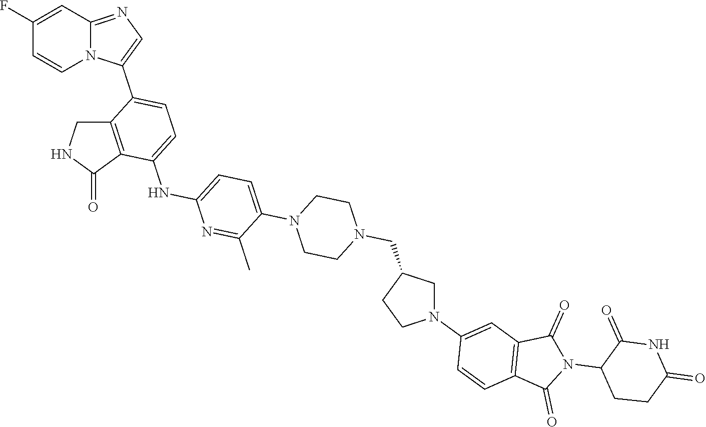 [SMILES: Cc1nc(Nc2ccc(-c3cnc4cc(F)ccn34)c3c2C(=O)NC3)ccc1N1CCN(C[C@H]2CCN(c3ccc4c(c3)C(=O)N(C3CCC(=O)NC3=O)C4=O)C2)CC1] |
| 104 | 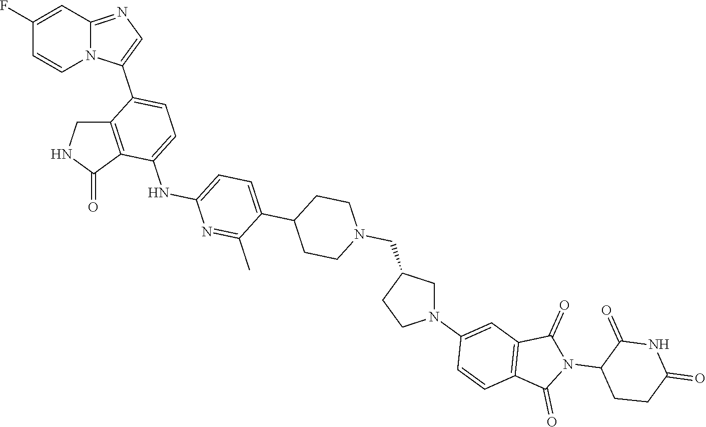 [SMILES: Cc1nc(Nc2ccc(-c3cnc4cc(F)ccn34)c3c2C(=O)NC3)ccc1C1CCN(C[C@H]2CCN(c3ccc4c(c3)C(=O)N(C3CCC(=O)NC3=O)C4=O)C2)CC1] |
| 105 | 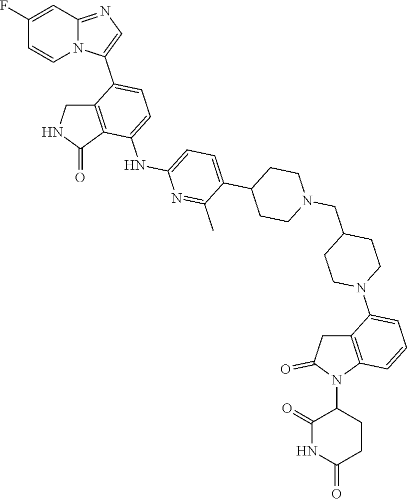 [SMILES: Cc1nc(Nc2ccc(-c3cnc4cc(F)ccn34)c3c2C(=O)NC3)ccc1C1CCN(CC2CCN(c3cccc4c3CC(=O)N4C3CCC(=O)NC3=O)CC2)CC1] |
| 106 | 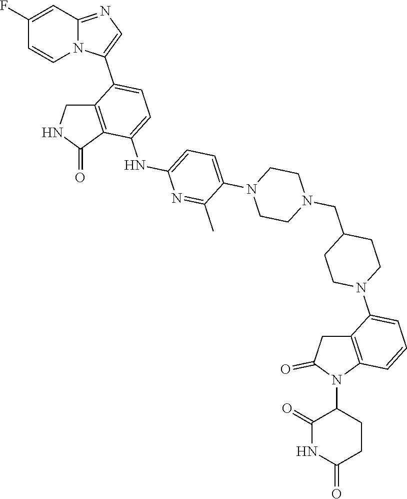 [SMILES: Cc1nc(Nc2ccc(-c3cnc4cc(F)ccn34)c3c2C(=O)NC3)ccc1N1CCN(CC2CCN(c3cccc4c3CC(=O)N4C3CCC(=O)NC3=O)CC2)CC1] |
| 107 | 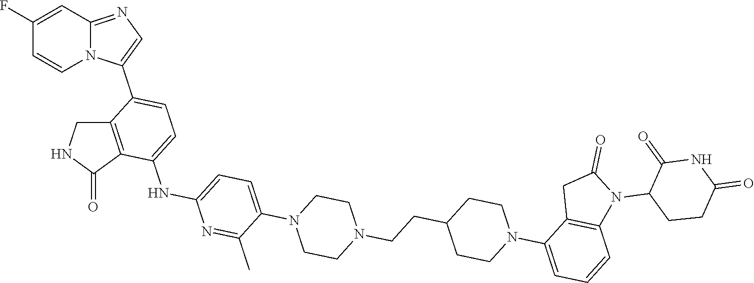 [SMILES: Cc1nc(Nc2ccc(-c3cnc4cc(F)ccn34)c3c2C(=O)NC3)ccc1N1CCN(CCC2CCN(c3cccc4c3CC(=O)N4C3CCC(=O)NC3=O)CC2)CC1] |
| 108 | 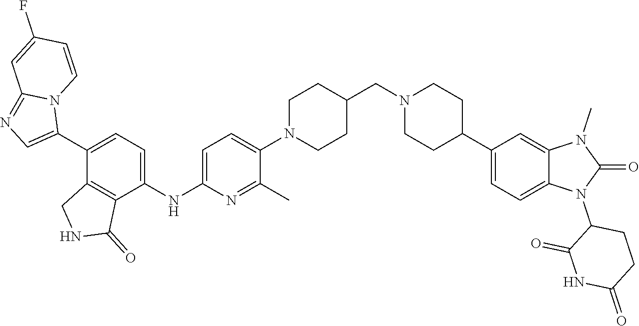 [SMILES: Cc1nc(Nc2ccc(-c3cnc4cc(F)ccn34)c3c2C(=O)NC3)ccc1N1CCC(CN2CCC(c3ccc4c(c3)n(C)c(=O)n4C3CCC(=O)NC3=O)CC2)CC1] |
| 109 | 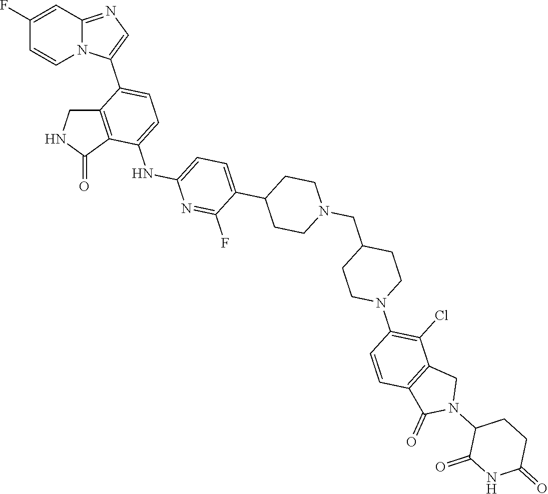 [SMILES: O=C1CCC(N2Cc3c(ccc(N4CCC(CN5CCC(c6ccc(Nc7ccc(-c8cnc9cc(F)ccn89)c8c7C(=O)NC8)nc6F)CC5)CC4)c3Cl)C2=O)C(=O)N1] |
| 110 | 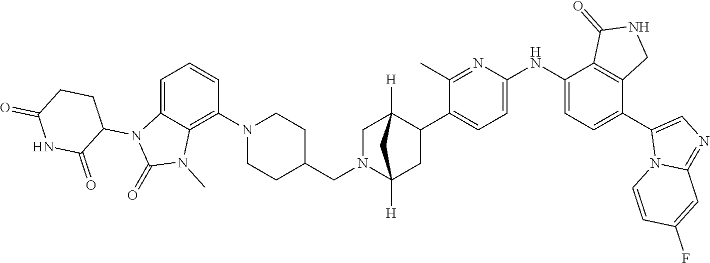 [SMILES: Cc1nc(Nc2ccc(-c3cnc4cc(F)ccn34)c3c2C(=O)NC3)ccc1C1C[C@H]2C[C@@H]1CN2CC1CCN(c2cccc3c2n(C)c(=O)n3C2CCC(=O)NC2=O)CC1] |
| 111 | 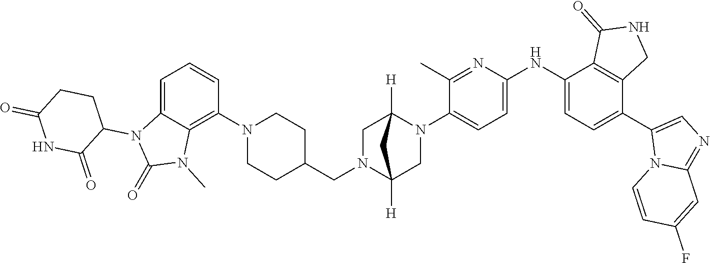 [SMILES: Cc1nc(Nc2ccc(-c3cnc4cc(F)ccn34)c3c2C(=O)NC3)ccc1N1C[C@H]2C[C@@H]1CN2CC1CCN(c2cccc3c2n(C)c(=O)n3C2CCC(=O)NC2=O)CC1] |
| 112 | 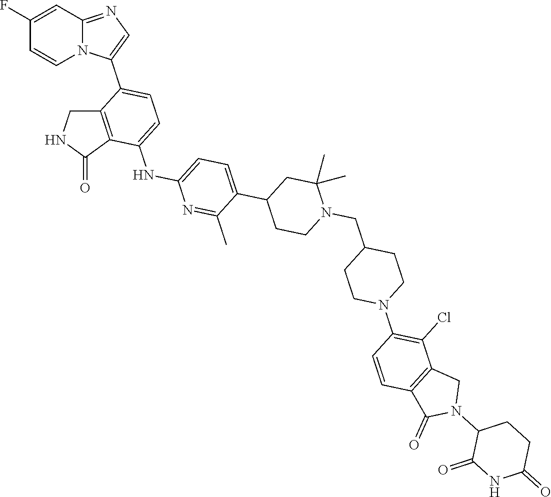 [SMILES: Cc1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)C(C)(C)C1] |
| 113 | 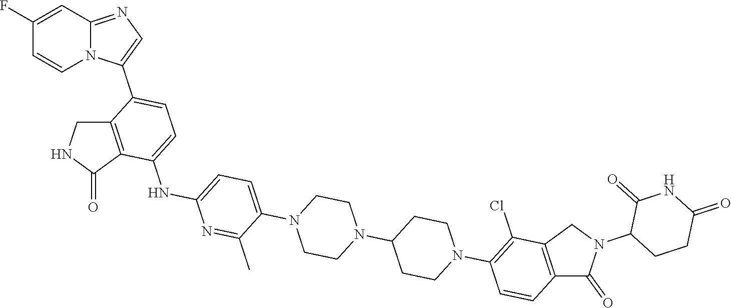 [SMILES: Cc1nc(Nc2ccc(-c3cnc4cc(F)ccn34)c3c2C(=O)NC3)ccc1N1CCN(C2CCN(c3ccc4c(c3Cl)CN(C3CCC(=O)NC3=O)C4=O)CC2)CC1] |
| 114 | 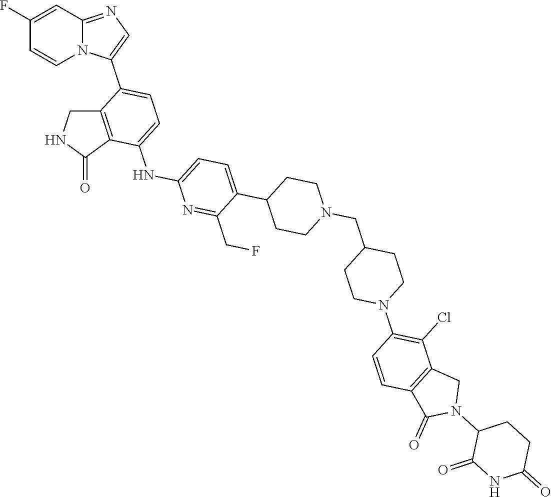 [SMILES: O=C1CCC(N2Cc3c(ccc(N4CCC(CN5CCC(c6ccc(Nc7ccc(-c8cnc9cc(F)ccn89)c8c7C(=O)NC8)nc6CF)CC5)CC4)c3Cl)C2=O)C(=O)N1] |
| 115 | 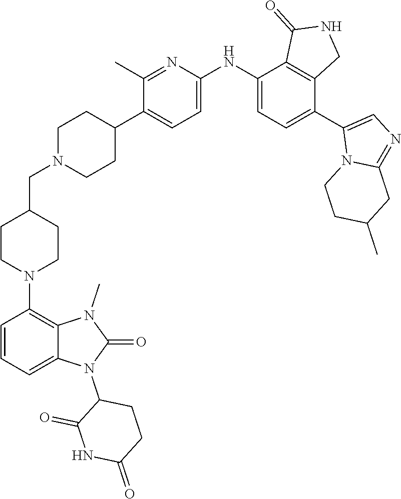 [SMILES: Cc1nc(Nc2ccc(-c3cnc4n3CCC(C)C4)c3c2C(=O)NC3)ccc1C1CCN(CC2CCN(c3cccc4c3n(C)c(=O)n4C3CCC(=O)NC3=O)CC2)CC1] |
| 116 | 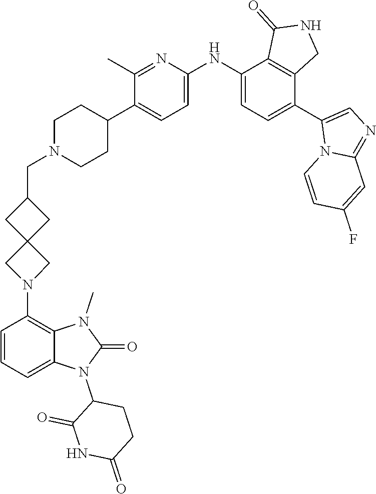 [SMILES: Cc1nc(Nc2ccc(-c3cnc4cc(F)ccn34)c3c2C(=O)NC3)ccc1C1CCN(CC2CC3(C2)CN(c2cccc4c2n(C)c(=O)n4C2CCC(=O)NC2=O)C3)CC1] |
| 117 | 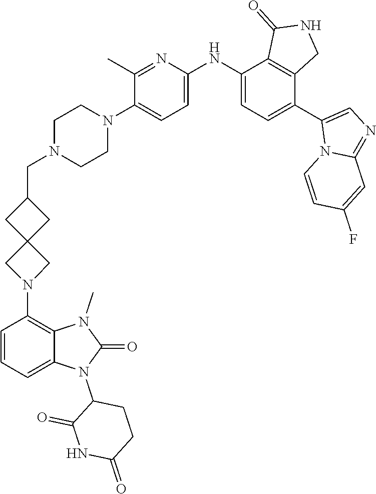 [SMILES: Cc1nc(Nc2ccc(-c3cnc4cc(F)ccn34)c3c2C(=O)NC3)ccc1N1CCN(CC2CC3(C2)CN(c2cccc4c2n(C)c(=O)n4C2CCC(=O)NC2=O)C3)CC1] |
| 118 | 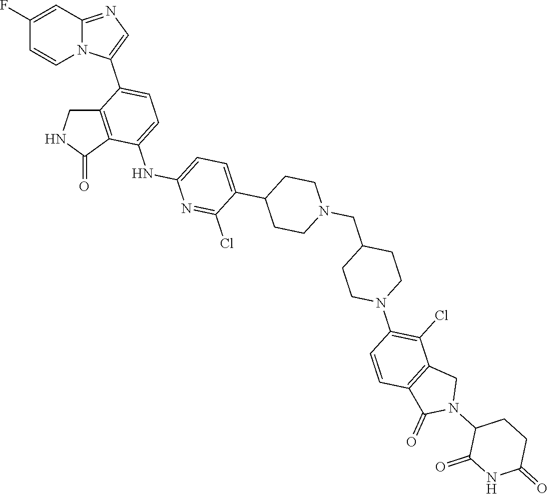 [SMILES: O=C1CCC(N2Cc3c(ccc(N4CCC(CN5CCC(c6ccc(Nc7ccc(-c8cnc9cc(F)ccn89)c8c7C(=O)NC8)nc6Cl)CC5)CC4)c3Cl)C2=O)C(=O)N1] |
| 119 | 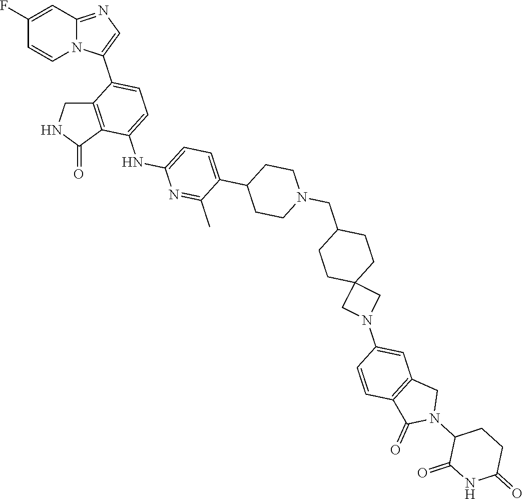 [SMILES: Cc1nc(Nc2ccc(-c3cnc4cc(F)ccn34)c3c2C(=O)NC3)ccc1C1CCN(CC2CCC3(CC2)CN(c2ccc4c(c2)CN(C2CCC(=O)NC2=O)C4=O)C3)CC1] |
| 120 | 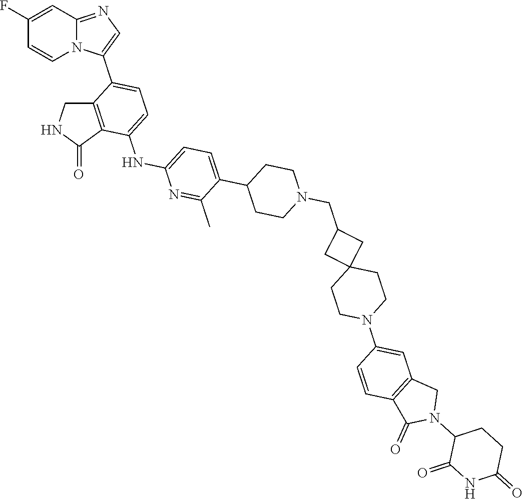 [SMILES: Cc1nc(Nc2ccc(-c3cnc4cc(F)ccn34)c3c2C(=O)NC3)ccc1C1CCN(CC2CC3(CCN(c4ccc5c(c4)CN(C4CCC(=O)NC4=O)C5=O)CC3)C2)CC1] |
| 121 | 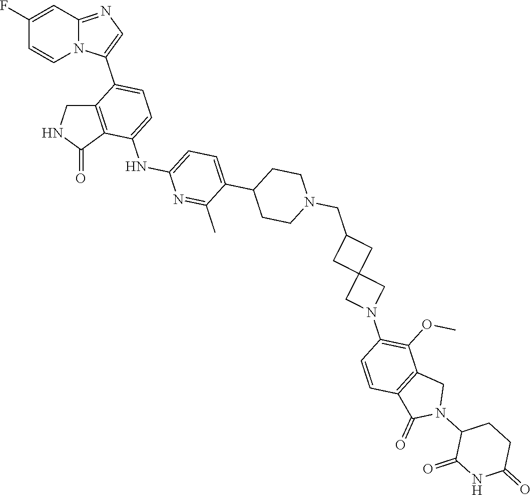 [SMILES: COc1c(N2CC3(CC(CN4CCC(c5ccc(Nc6ccc(-c7cnc8cc(F)ccn78)c7c6C(=O)NC7)nc5C)CC4)C3)C2)ccc2c1CN(C1CCC(=O)NC1=O)C2=O] |
| 122 | 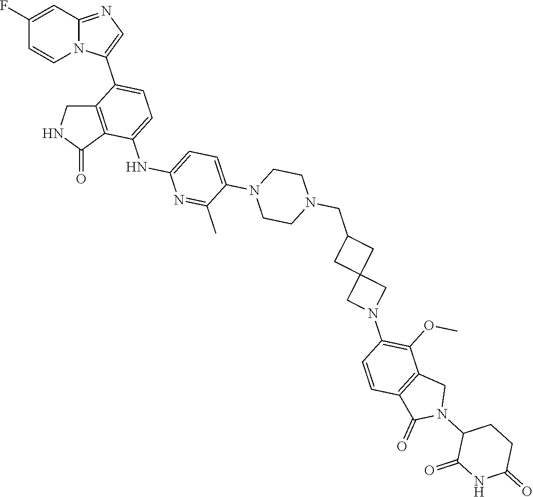 [SMILES: COc1c(N2CC3(CC(CN4CCN(c5ccc(Nc6ccc(-c7cnc8cc(F)ccn78)c7c6C(=O)NC7)nc5C)CC4)C3)C2)ccc2c1CN(C1CCC(=O)NC1=O)C2=O] |
| 123 | 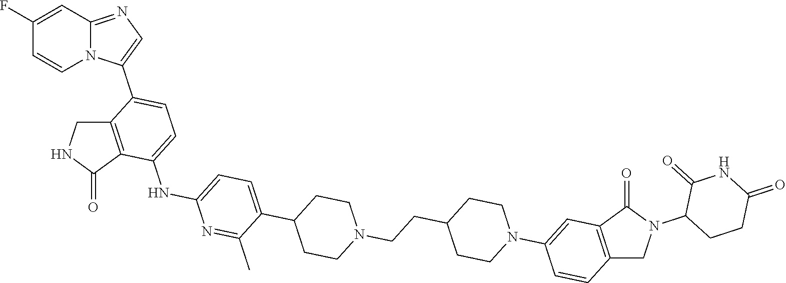 [SMILES: Cc1nc(Nc2ccc(-c3cnc4cc(F)ccn34)c3c2C(=O)NC3)ccc1C1CCN(CCC2CCN(c3ccc4c(c3)C(=O)N(C3CCC(=O)NC3=O)C4)CC2)CC1] |
| 124 | 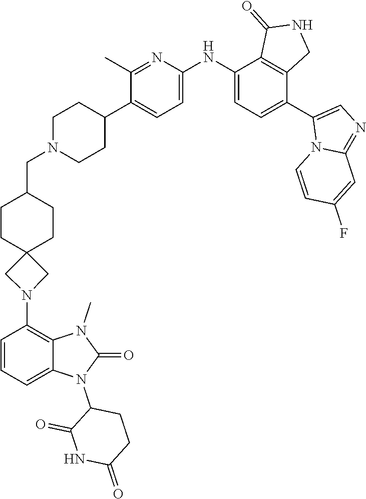 [SMILES: Cc1nc(Nc2ccc(-c3cnc4cc(F)ccn34)c3c2C(=O)NC3)ccc1C1CCN(CC2CCC3(CC2)CN(c2cccc4c2n(C)c(=O)n4C2CCC(=O)NC2=O)C3)CC1] |
| 125 | 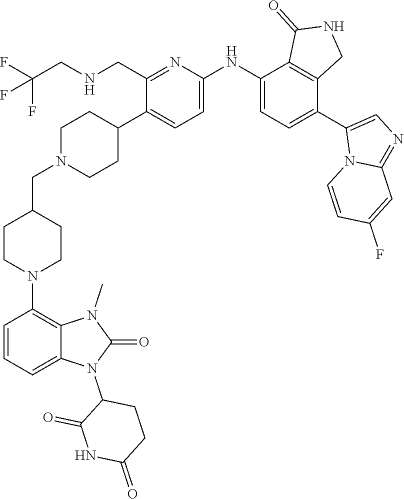 [SMILES: Cn1c(=O)n(C2CCC(=O)NC2=O)c2cccc(N3CCC(CN4CCC(c5ccc(Nc6ccc(-c7cnc8cc(F)ccn78)c7c6C(=O)NC7)nc5CNCC(F)(F)F)CC4)CC3)c21] |
| 126 | 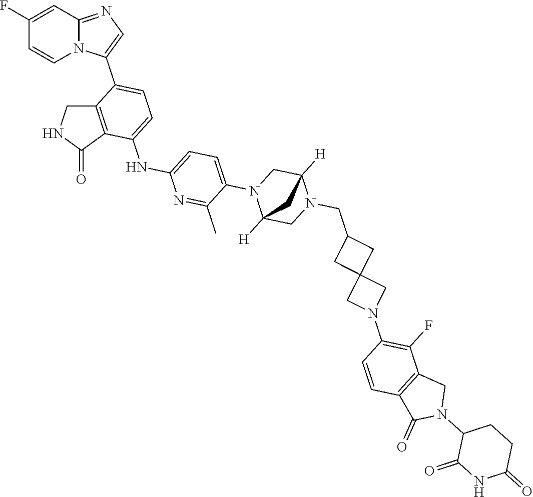 [SMILES: Cc1nc(Nc2ccc(-c3cnc4cc(F)ccn34)c3c2C(=O)NC3)ccc1N1C[C@H]2C[C@@H]1CN2CC1CC2(C1)CN(c1ccc3c(c1F)CN(C1CCC(=O)NC1=O)C3=O)C2] |
| 127 | 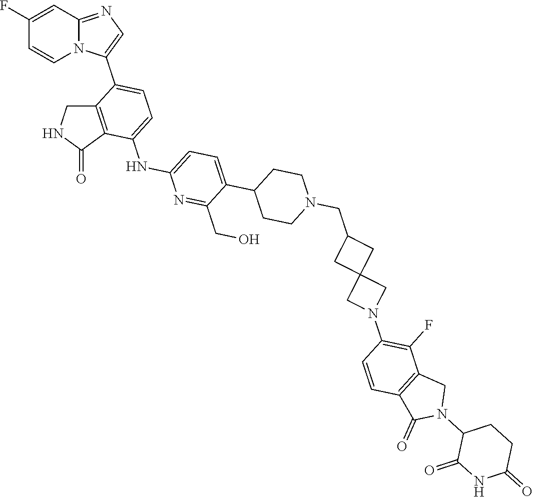 [SMILES: O=C1CCC(N2Cc3c(ccc(N4CC5(CC(CN6CCC(c7ccc(Nc8ccc(-c9cnc%10cc(F)ccn9%10)c9c8C(=O)NC9)nc7CO)CC6)C5)C4)c3F)C2=O)C(=O)N1] |
| 128 | 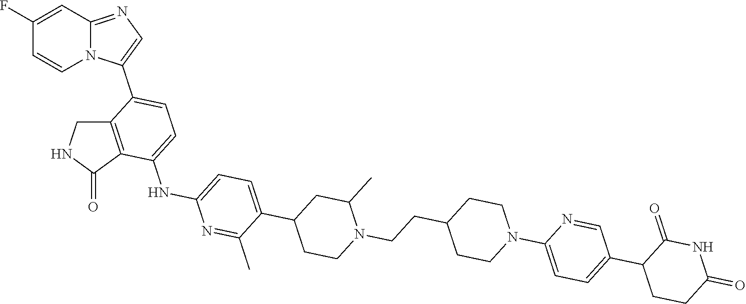 [SMILES: Cc1nc(Nc2ccc(-c3cnc4cc(F)ccn34)c3c2C(=O)NC3)ccc1C1CCN(CCC2CCN(c3ccc(C4CCC(=O)NC4=O)cn3)CC2)C(C)C1] |
| 129 | 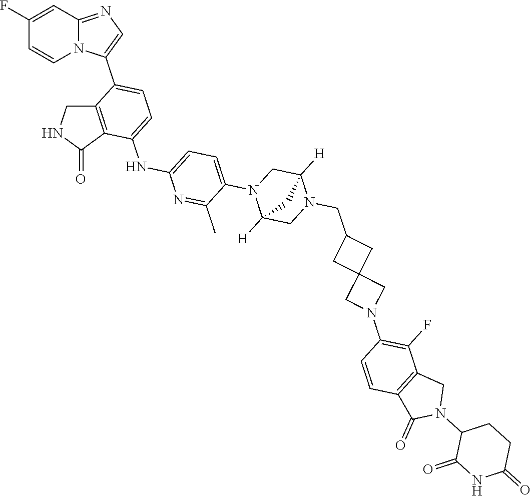 [SMILES: Cc1nc(Nc2ccc(-c3cnc4cc(F)ccn34)c3c2C(=O)NC3)ccc1N1C[C@@H]2C[C@H]1CN2CC1CC2(C1)CN(c1ccc3c(c1F)CN(C1CCC(=O)NC1=O)C3=O)C2] |
| 130 | 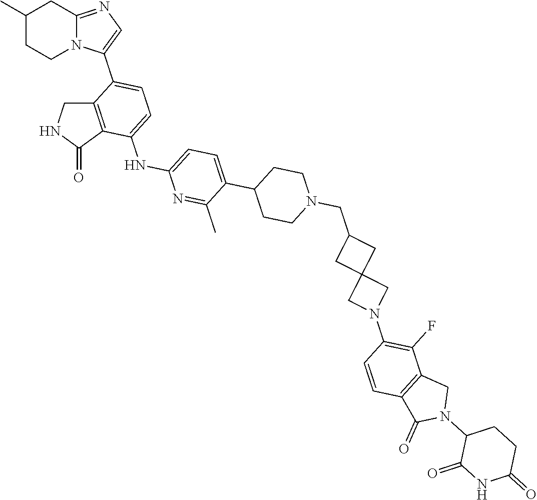 [SMILES: Cc1nc(Nc2ccc(-c3cnc4n3CCC(C)C4)c3c2C(=O)NC3)ccc1C1CCN(CC2CC3(C2)CN(c2ccc4c(c2F)CN(C2CCC(=O)NC2=O)C4=O)C3)CC1] |
| 131 | 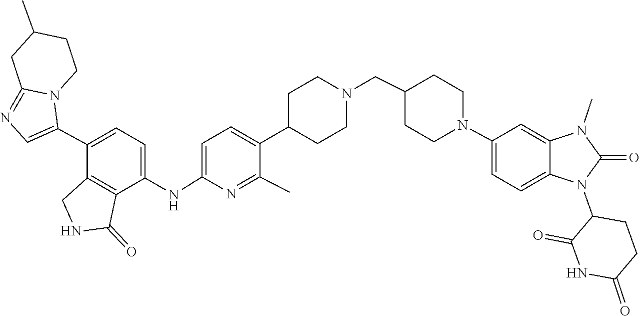 [SMILES: Cc1nc(Nc2ccc(-c3cnc4n3CCC(C)C4)c3c2C(=O)NC3)ccc1C1CCN(CC2CCN(c3ccc4c(c3)n(C)c(=O)n4C3CCC(=O)NC3=O)CC2)CC1] |
| 132 | 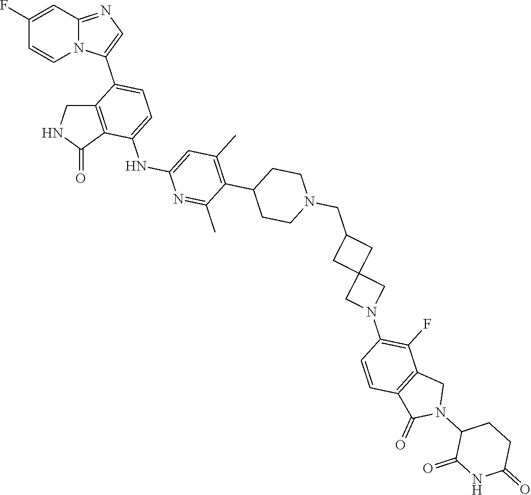 [SMILES: Cc1cc(Nc2ccc(-c3cnc4cc(F)ccn34)c3c2C(=O)NC3)nc(C)c1C1CCN(CC2CC3(C2)CN(c2ccc4c(c2F)CN(C2CCC(=O)NC2=O)C4=O)C3)CC1] |
| 133 | 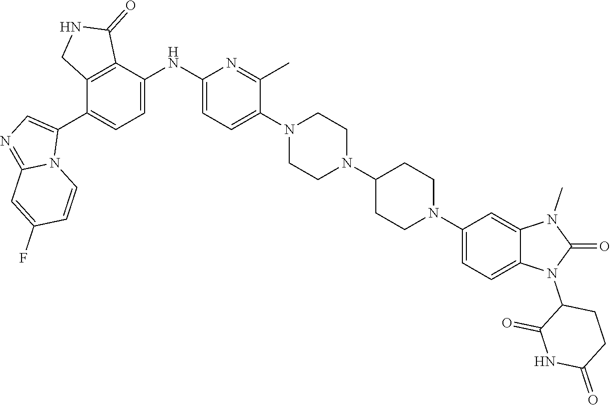 [SMILES: Cc1nc(Nc2ccc(-c3cnc4cc(F)ccn34)c3c2C(=O)NC3)ccc1N1CCN(C2CCN(c3ccc4c(c3)n(C)c(=O)n4C3CCC(=O)NC3=O)CC2)CC1] |
| 134 | 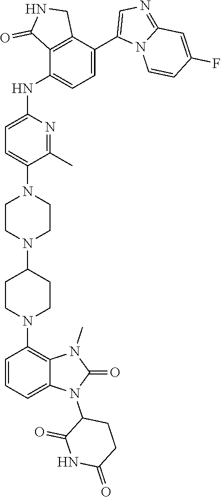 [SMILES: Cc1nc(Nc2ccc(-c3cnc4cc(F)ccn34)c3c2C(=O)NC3)ccc1N1CCN(C2CCN(c3cccc4c3n(C)c(=O)n4C3CCC(=O)NC3=O)CC2)CC1] |
| 135 | 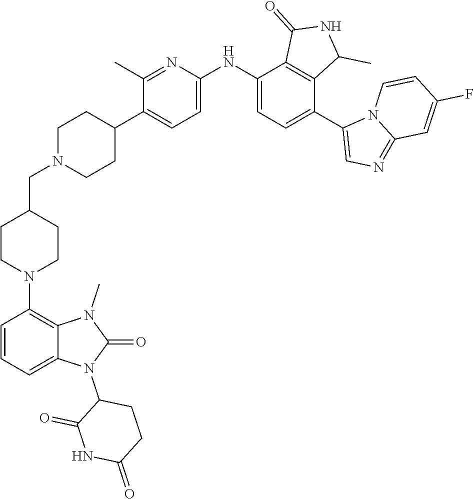 [SMILES: Cc1nc(Nc2ccc(-c3cnc4cc(F)ccn34)c3c2C(=O)NC3C)ccc1C1CCN(CC2CCN(c3cccc4c3n(C)c(=O)n4C3CCC(=O)NC3=O)CC2)CC1] |
| 136 | 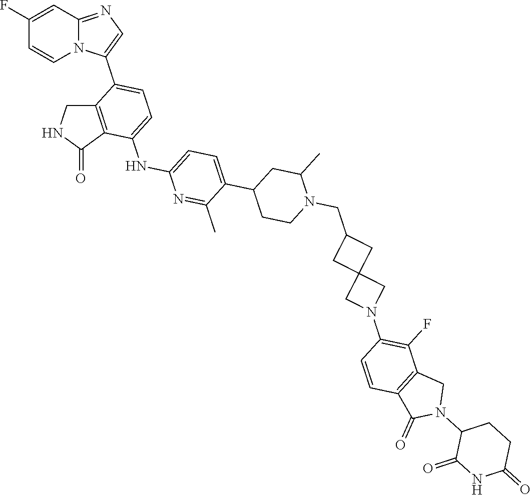 [SMILES: Cc1nc(Nc2ccc(-c3cnc4cc(F)ccn34)c3c2C(=O)NC3)ccc1C1CCN(CC2CC3(C2)CN(c2ccc4c(c2F)CN(C2CCC(=O)NC2=O)C4=O)C3)C(C)C1] |
| 137 | 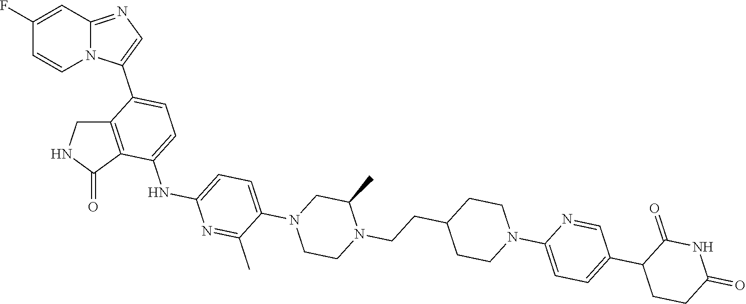 [SMILES: Cc1nc(Nc2ccc(-c3cnc4cc(F)ccn34)c3c2C(=O)NC3)ccc1N1CCN(CCC2CCN(c3ccc(C4CCC(=O)NC4=O)cn3)CC2)[C@H](C)C1] |
| 138 | 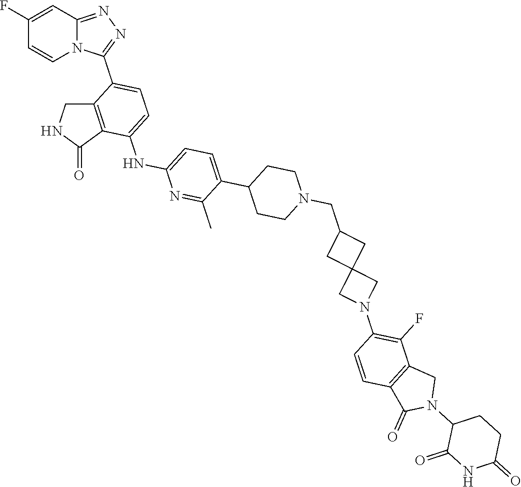 [SMILES: Cc1nc(Nc2ccc(-c3nnc4cc(F)ccn34)c3c2C(=O)NC3)ccc1C1CCN(CC2CC3(C2)CN(c2ccc4c(c2F)CN(C2CCC(=O)NC2=O)C4=O)C3)CC1] |
| 139 | 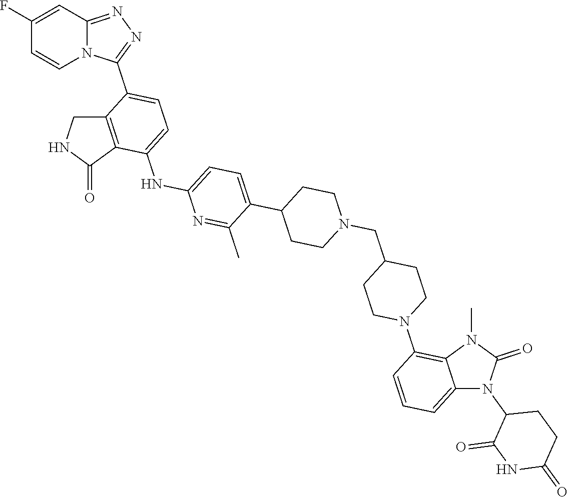 [SMILES: Cc1nc(Nc2ccc(-c3nnc4cc(F)ccn34)c3c2C(=O)NC3)ccc1C1CCN(CC2CCN(c3cccc4c3n(C)c(=O)n4C3CCC(=O)NC3=O)CC2)CC1] |
| 140 | 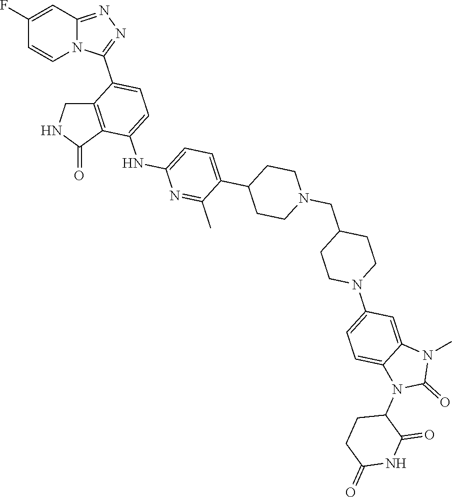 [SMILES: Cc1nc(Nc2ccc(-c3nnc4cc(F)ccn34)c3c2C(=O)NC3)ccc1C1CCN(CC2CCN(c3ccc4c(c3)n(C)c(=O)n4C3CCC(=O)NC3=O)CC2)CC1] |
| 141 | 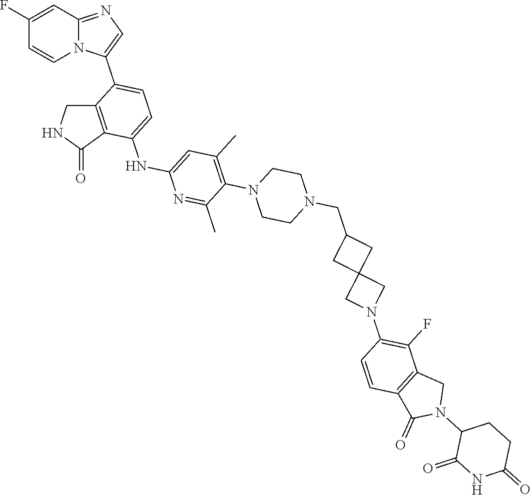 [SMILES: Cc1cc(Nc2ccc(-c3cnc4cc(F)ccn34)c3c2C(=O)NC3)nc(C)c1N1CCN(CC2CC3(C2)CN(c2ccc4c(c2F)CN(C2CCC(=O)NC2=O)C4=O)C3)CC1] |
| 142 | 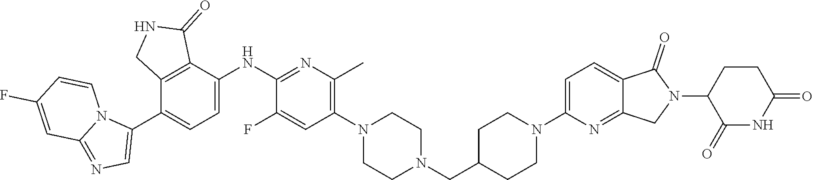 [SMILES: Cc1nc(Nc2ccc(-c3cnc4cc(F)ccn34)c3c2C(=O)NC3)c(F)cc1N1CCN(CC2CCN(c3ccc4c(n3)CN(C3CCC(=O)NC3=O)C4=O)CC2)CC1] |
| 143 | 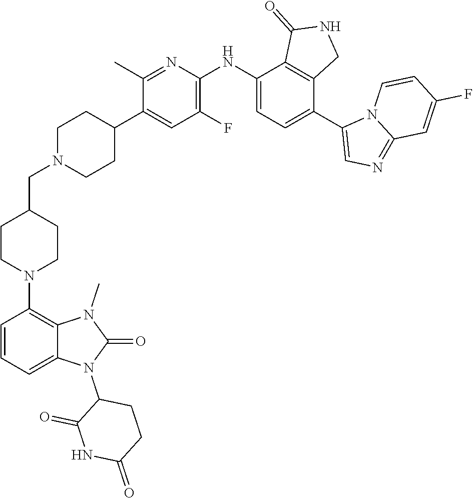 [SMILES: Cc1nc(Nc2ccc(-c3cnc4cc(F)ccn34)c3c2C(=O)NC3)c(F)cc1C1CCN(CC2CCN(c3cccc4c3n(C)c(=O)n4C3CCC(=O)NC3=O)CC2)CC1] |
| 144 | 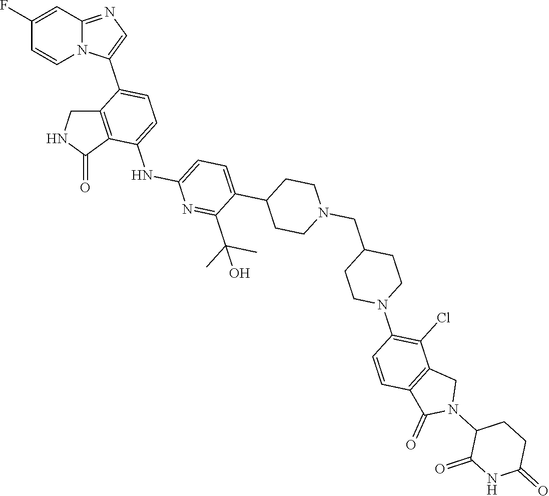 [SMILES: CC(C)(O)c1nc(Nc2ccc(-c3cnc4cc(F)ccn34)c3c2C(=O)NC3)ccc1C1CCN(CC2CCN(c3ccc4c(c3Cl)CN(C3CCC(=O)NC3=O)C4=O)CC2)CC1] |
| 145 | 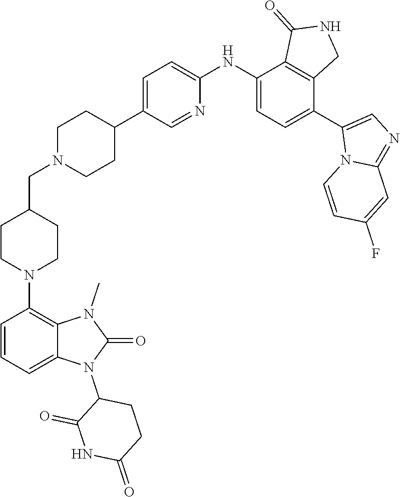 [SMILES: Cn1c(=O)n(C2CCC(=O)NC2=O)c2cccc(N3CCC(CN4CCC(c5ccc(Nc6ccc(-c7cnc8cc(F)ccn78)c7c6C(=O)NC7)nc5)CC4)CC3)c21] |
| 146 | 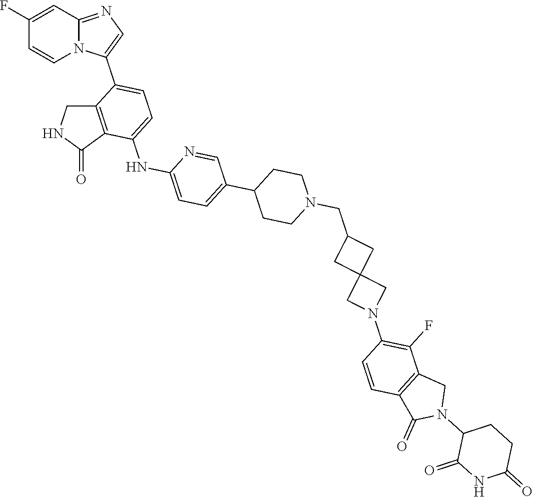 [SMILES: O=C1CCC(N2Cc3c(ccc(N4CC5(CC(CN6CCC(c7ccc(Nc8ccc(-c9cnc%10cc(F)ccn9%10)c9c8C(=O)NC9)nc7)CC6)C5)C4)c3F)C2=O)C(=O)N1] |
| 147 | 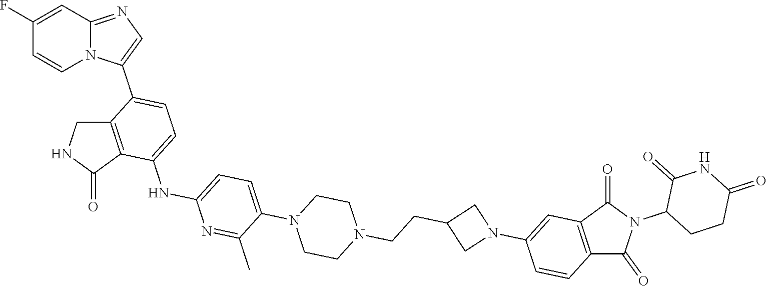 [SMILES: Cc1nc(Nc2ccc(-c3cnc4cc(F)ccn34)c3c2C(=O)NC3)ccc1N1CCN(CCC2CN(c3ccc4c(c3)C(=O)N(C3CCC(=O)NC3=O)C4=O)C2)CC1] |
| 148 | 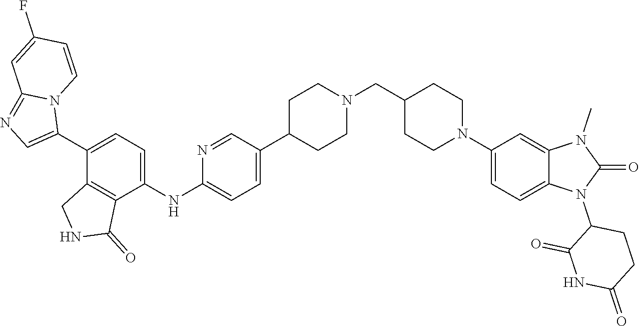 [SMILES: Cn1c(=O)n(C2CCC(=O)NC2=O)c2ccc(N3CCC(CN4CCC(c5ccc(Nc6ccc(-c7cnc8cc(F)ccn78)c7c6C(=O)NC7)nc5)CC4)CC3)cc21] |
| 149 | 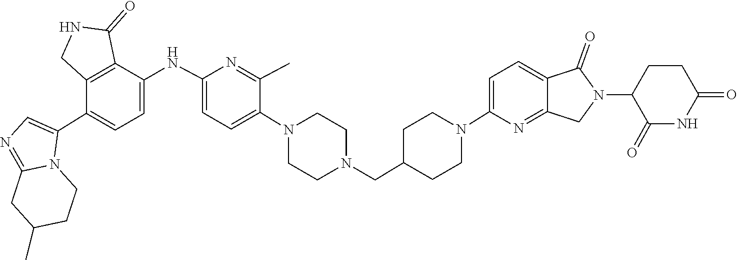 [SMILES: Cc1nc(Nc2ccc(-c3cnc4n3CCC(C)C4)c3c2C(=O)NC3)ccc1N1CCN(CC2CCN(c3ccc4c(n3)CN(C3CCC(=O)NC3=O)C4=O)CC2)CC1] |
| 150 | 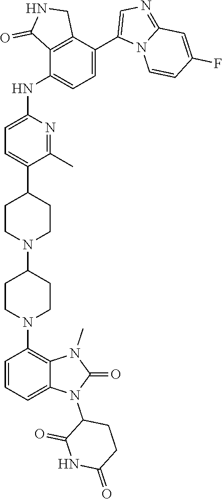 [SMILES: Cc1nc(Nc2ccc(-c3cnc4cc(F)ccn34)c3c2C(=O)NC3)ccc1C1CCN(C2CCN(c3cccc4c3n(C)c(=O)n4C3CCC(=O)NC3=O)CC2)CC1] |
| 151 | 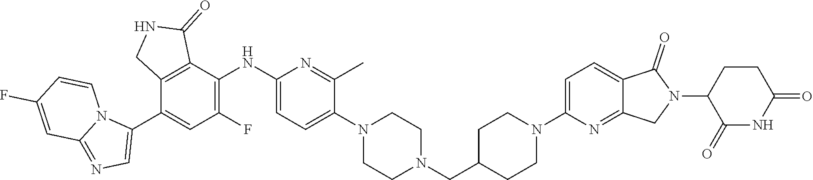 [SMILES: Cc1nc(Nc2c(F)cc(-c3cnc4cc(F)ccn34)c3c2C(=O)NC3)ccc1N1CCN(CC2CCN(c3ccc4c(n3)CN(C3CCC(=O)NC3=O)C4=O)CC2)CC1] |
| 152 | 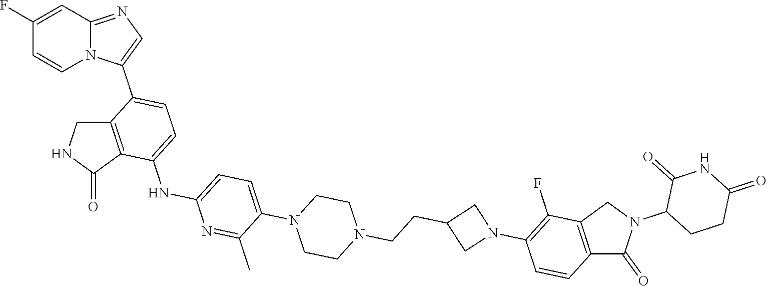 [SMILES: Cc1nc(Nc2ccc(-c3cnc4cc(F)ccn34)c3c2C(=O)NC3)ccc1N1CCN(CCC2CN(c3ccc4c(c3F)CN(C3CCC(=O)NC3=O)C4=O)C2)CC1] |
| 153 | 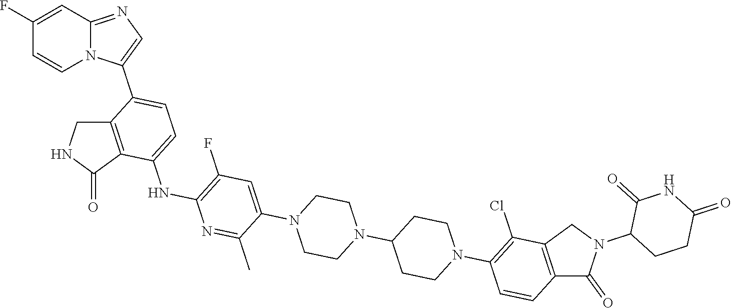 [SMILES: Cc1nc(Nc2ccc(-c3cnc4cc(F)ccn34)c3c2C(=O)NC3)c(F)cc1N1CCN(C2CCN(c3ccc4c(c3Cl)CN(C3CCC(=O)NC3=O)C4=O)CC2)CC1] |
| 154 | 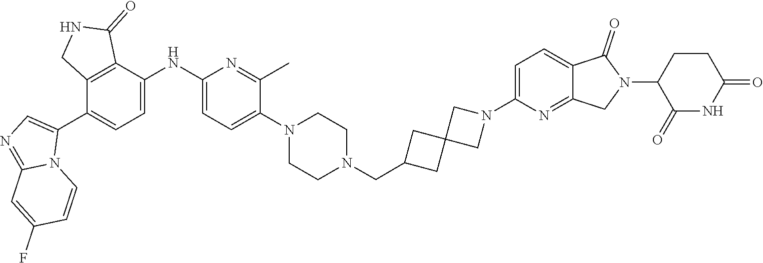 [SMILES: Cc1nc(Nc2ccc(-c3cnc4cc(F)ccn34)c3c2C(=O)NC3)ccc1N1CCN(CC2CC3(C2)CN(c2ccc4c(n2)CN(C2CCC(=O)NC2=O)C4=O)C3)CC1] |
| 155 | 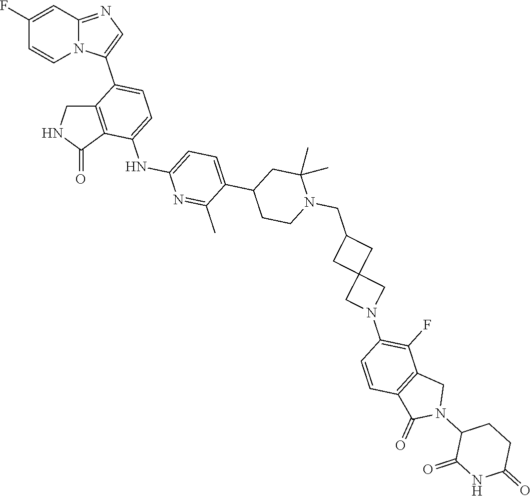 [SMILES: Cc1nc(Nc2ccc(-c3cnc4cc(F)ccn34)c3c2C(=O)NC3)ccc1C1CCN(CC2CC3(C2)CN(c2ccc4c(c2F)CN(C2CCC(=O)NC2=O)C4=O)C3)C(C)(C)C1] |
| 156 | 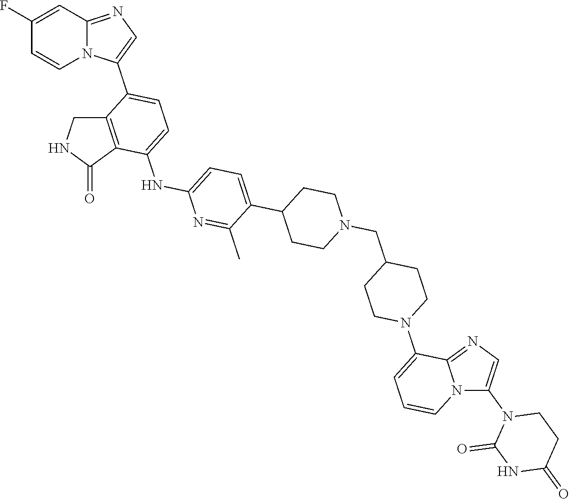 [SMILES: Cc1nc(Nc2ccc(-c3cnc4cc(F)ccn34)c3c2C(=O)NC3)ccc1C1CCN(CC2CCN(c3cccn4c(N5CCC(=O)NC5=O)cnc34)CC2)CC1] |
| 157 | 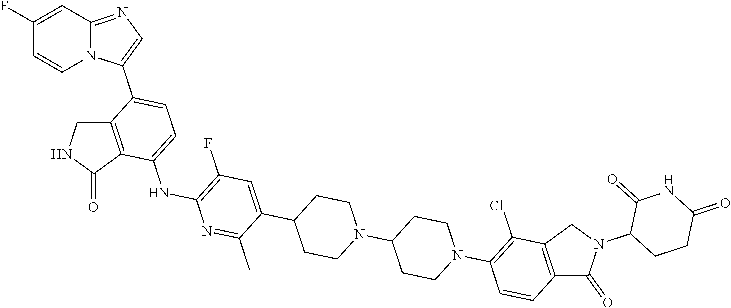 [SMILES: Cc1nc(Nc2ccc(-c3cnc4cc(F)ccn34)c3c2C(=O)NC3)c(F)cc1C1CCN(C2CCN(c3ccc4c(c3Cl)CN(C3CCC(=O)NC3=O)C4=O)CC2)CC1] |
| 158 | 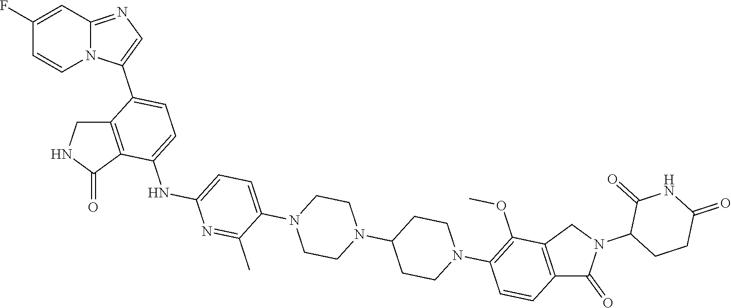 [SMILES: COc1c(N2CCC(N3CCN(c4ccc(Nc5ccc(-c6cnc7cc(F)ccn67)c6c5C(=O)NC6)nc4C)CC3)CC2)ccc2c1CN(C1CCC(=O)NC1=O)C2=O] |
| 159 | 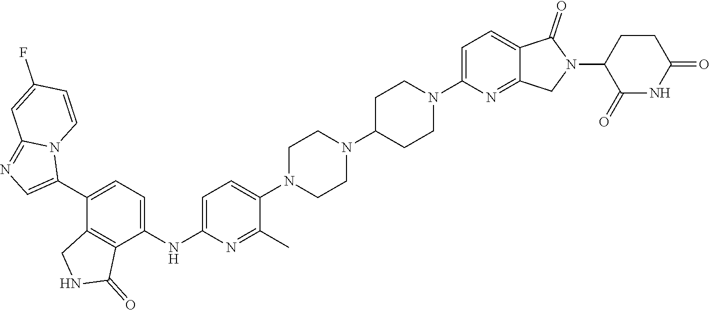 [SMILES: Cc1nc(Nc2ccc(-c3cnc4cc(F)ccn34)c3c2C(=O)NC3)ccc1N1CCN(C2CCN(c3ccc4c(n3)CN(C3CCC(=O)NC3=O)C4=O)CC2)CC1] |
| 160 | 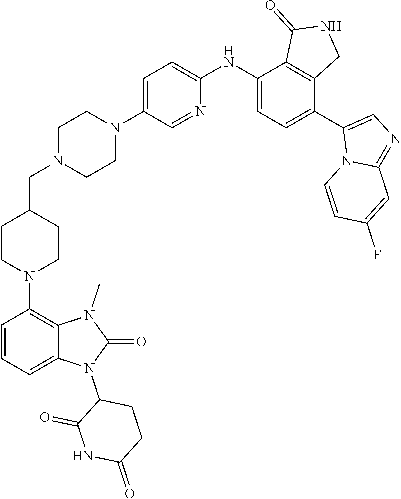 [SMILES: Cn1c(=O)n(C2CCC(=O)NC2=O)c2cccc(N3CCC(CN4CCN(c5ccc(Nc6ccc(-c7cnc8cc(F)ccn78)c7c6C(=O)NC7)nc5)CC4)CC3)c21] |
| 161 | 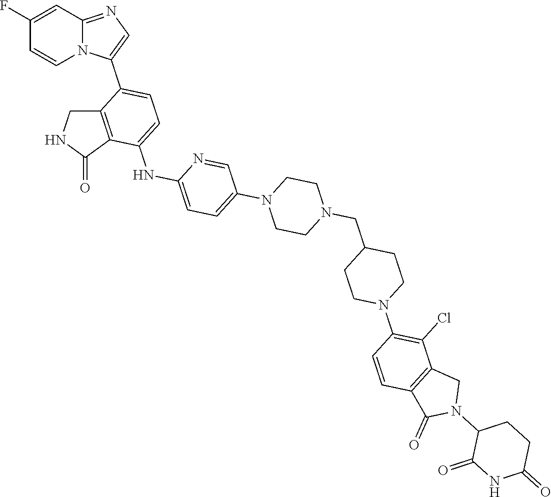 [SMILES: O=C1CCC(N2Cc3c(ccc(N4CCC(CN5CCN(c6ccc(Nc7ccc(-c8cnc9cc(F)ccn89)c8c7C(=O)NC8)nc6)CC5)CC4)c3Cl)C2=O)C(=O)N1] |
| 162 | 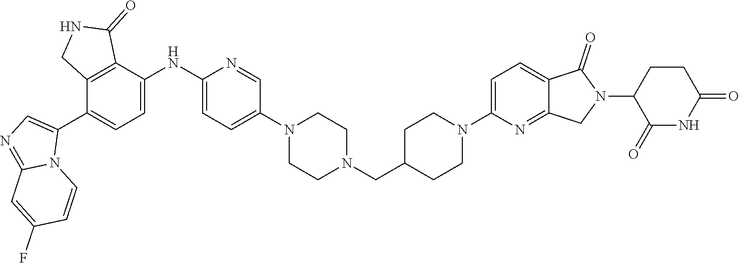 [SMILES: O=C1CCC(N2Cc3nc(N4CCC(CN5CCN(c6ccc(Nc7ccc(-c8cnc9cc(F)ccn89)c8c7C(=O)NC8)nc6)CC5)CC4)ccc3C2=O)C(=O)N1] |
| 163 | 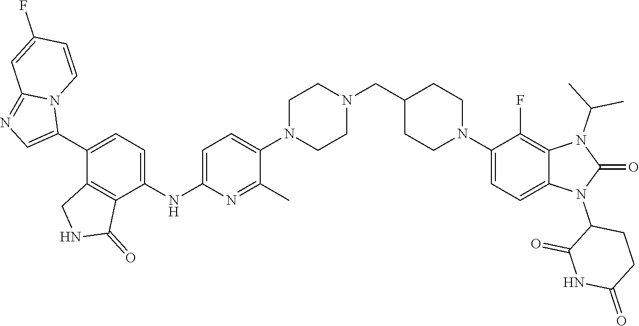 [SMILES: Cc1nc(Nc2ccc(-c3cnc4cc(F)ccn34)c3c2C(=O)NC3)ccc1N1CCN(CC2CCN(c3ccc4c(c3F)n(C(C)C)c(=O)n4C3CCC(=O)NC3=O)CC2)CC1] |
| 164 | 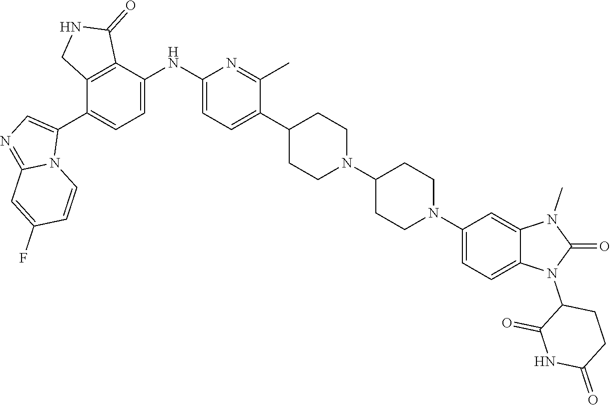 [SMILES: Cc1nc(Nc2ccc(-c3cnc4cc(F)ccn34)c3c2C(=O)NC3)ccc1C1CCN(C2CCN(c3ccc4c(c3)n(C)c(=O)n4C3CCC(=O)NC3=O)CC2)CC1] |
| 165 | 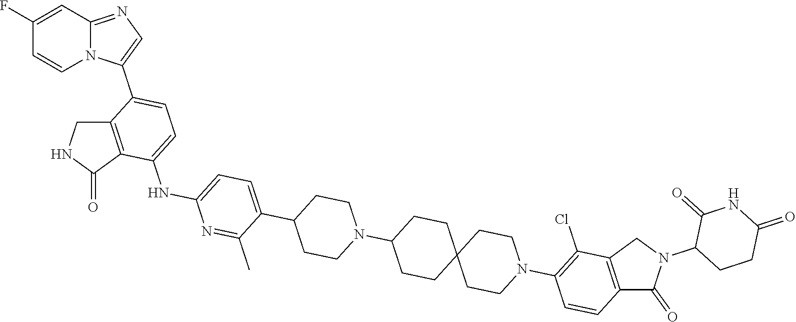 [SMILES: Cc1nc(Nc2ccc(-c3cnc4cc(F)ccn34)c3c2C(=O)NC3)ccc1C1CCN(C2CCC3(CC2)CCN(c2ccc4c(c2Cl)CN(C2CCC(=O)NC2=O)C4=O)CC3)CC1] |
| 166 | 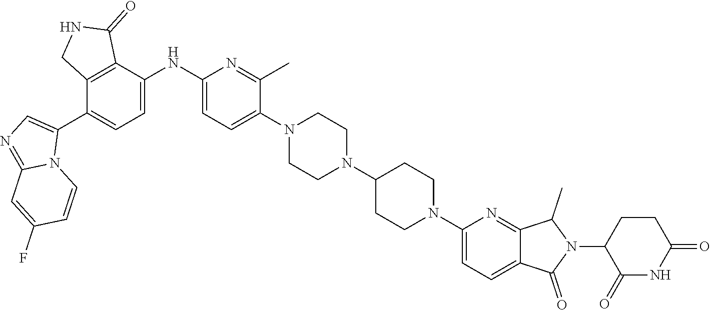 [SMILES: Cc1nc(Nc2ccc(-c3cnc4cc(F)ccn34)c3c2C(=O)NC3)ccc1N1CCN(C2CCN(c3ccc4c(n3)C(C)N(C3CCC(=O)NC3=O)C4=O)CC2)CC1] |
| 167 | 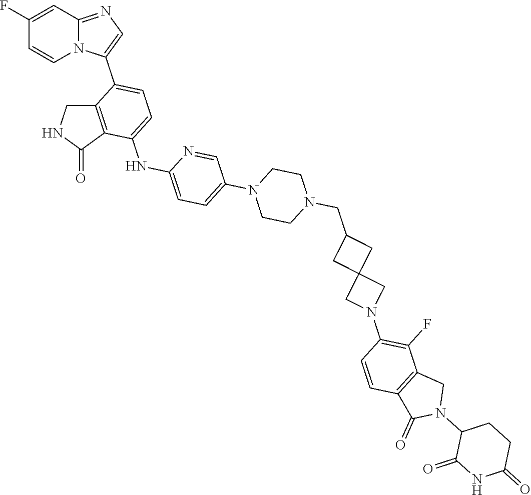 [SMILES: O=C1CCC(N2Cc3c(ccc(N4CC5(CC(CN6CCN(c7ccc(Nc8ccc(-c9cnc%10cc(F)ccn9%10)c9c8C(=O)NC9)nc7)CC6)C5)C4)c3F)C2=O)C(=O)N1] |
| 168 | 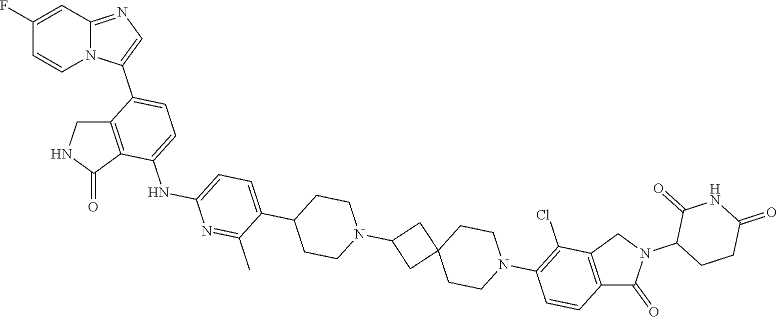 [SMILES: Cc1nc(Nc2ccc(-c3cnc4cc(F)ccn34)c3c2C(=O)NC3)ccc1C1CCN(C2CC3(CCN(c4ccc5c(c4Cl)CN(C4CCC(=O)NC4=O)C5=O)CC3)C2)CC1] |
| 169 | 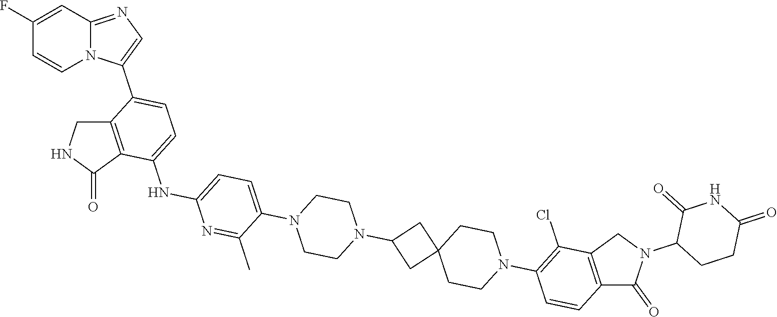 [SMILES: Cc1nc(Nc2ccc(-c3cnc4cc(F)ccn34)c3c2C(=O)NC3)ccc1N1CCN(C2CC3(CCN(c4ccc5c(c4Cl)CN(C4CCC(=O)NC4=O)C5=O)CC3)C2)CC1] |
| 170 | 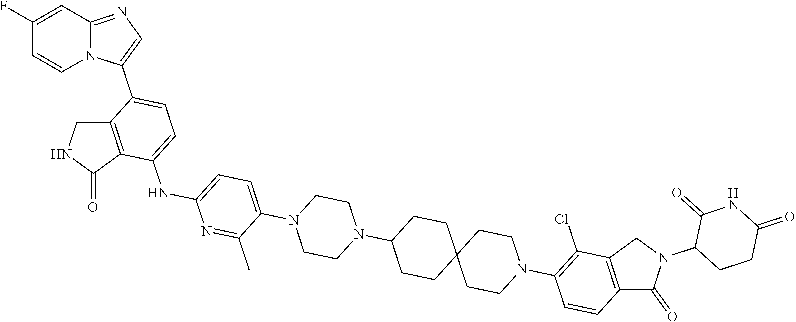 [SMILES: Cc1nc(Nc2ccc(-c3cnc4cc(F)ccn34)c3c2C(=O)NC3)ccc1N1CCN(C2CCC3(CC2)CCN(c2ccc4c(c2Cl)CN(C2CCC(=O)NC2=O)C4=O)CC3)CC1] |
| 171 | 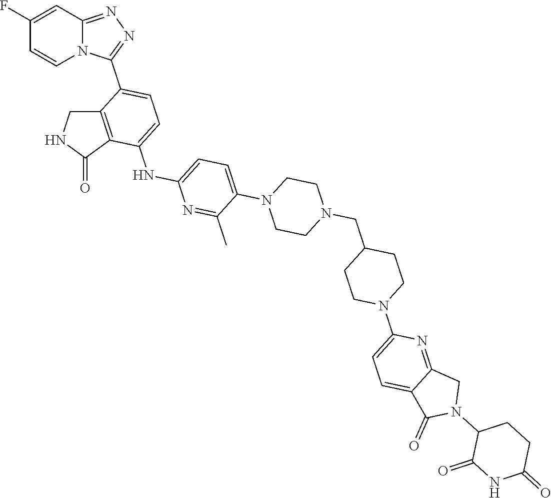 [SMILES: Cc1nc(Nc2ccc(-c3nnc4cc(F)ccn34)c3c2C(=O)NC3)ccc1N1CCN(CC2CCN(c3ccc4c(n3)CN(C3CCC(=O)NC3=O)C4=O)CC2)CC1] |
| 172 | 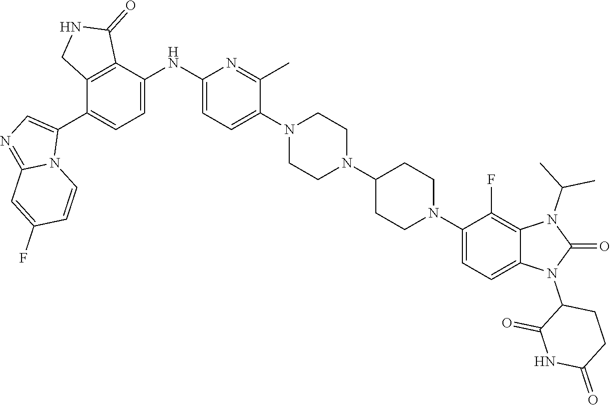 [SMILES: Cc1nc(Nc2ccc(-c3cnc4cc(F)ccn34)c3c2C(=O)NC3)ccc1N1CCN(C2CCN(c3ccc4c(c3F)n(C(C)C)c(=O)n4C3CCC(=O)NC3=O)CC2)CC1] |
| 173 | 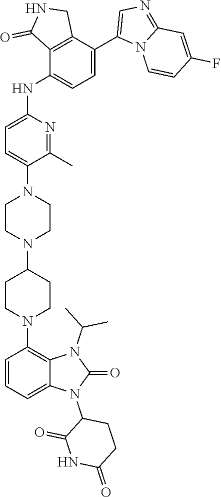 [SMILES: Cc1nc(Nc2ccc(-c3cnc4cc(F)ccn34)c3c2C(=O)NC3)ccc1N1CCN(C2CCN(c3cccc4c3n(C(C)C)c(=O)n4C3CCC(=O)NC3=O)CC2)CC1] |
| 174 | 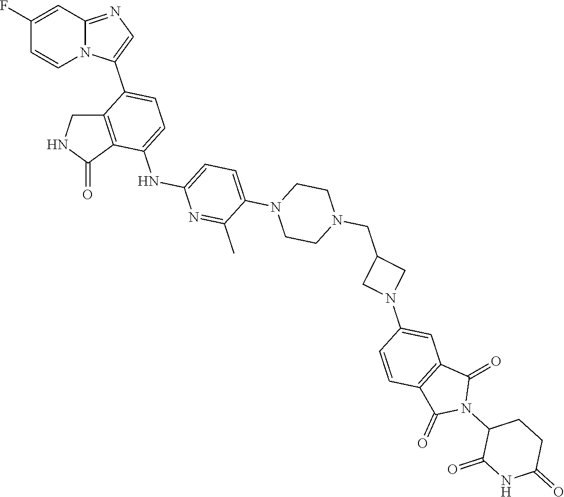 [SMILES: Cc1nc(Nc2ccc(-c3cnc4cc(F)ccn34)c3c2C(=O)NC3)ccc1N1CCN(CC2CN(c3ccc4c(c3)C(=O)N(C3CCC(=O)NC3=O)C4=O)C2)CC1] |
| 175 | 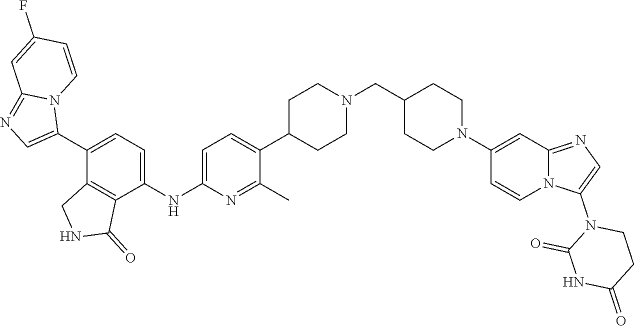 [SMILES: Cc1nc(Nc2ccc(-c3cnc4cc(F)ccn34)c3c2C(=O)NC3)ccc1C1CCN(CC2CCN(c3ccn4c(N5CCC(=O)NC5=O)cnc4c3)CC2)CC1] |
| 176 | 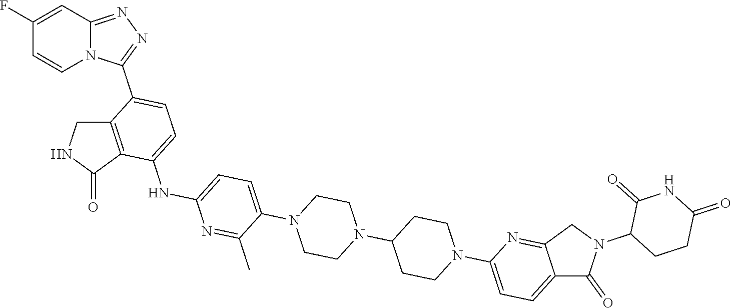 [SMILES: Cc1nc(Nc2ccc(-c3nnc4cc(F)ccn34)c3c2C(=O)NC3)ccc1N1CCN(C2CCN(c3ccc4c(n3)CN(C3CCC(=O)NC3=O)C4=O)CC2)CC1] |
| 177 | 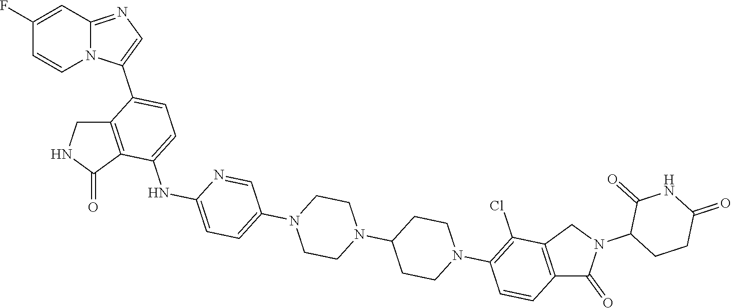 [SMILES: O=C1CCC(N2Cc3c(ccc(N4CCC(N5CCN(c6ccc(Nc7ccc(-c8cnc9cc(F)ccn89)c8c7C(=O)NC8)nc6)CC5)CC4)c3Cl)C2=O)C(=O)N1] |
| 178 | 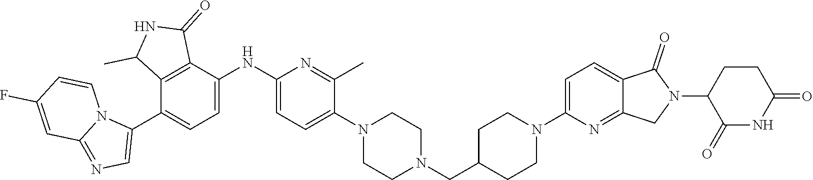 [SMILES: Cc1nc(Nc2ccc(-c3cnc4cc(F)ccn34)c3c2C(=O)NC3C)ccc1N1CCN(CC2CCN(c3ccc4c(n3)CN(C3CCC(=O)NC3=O)C4=O)CC2)CC1] |
| 179 | 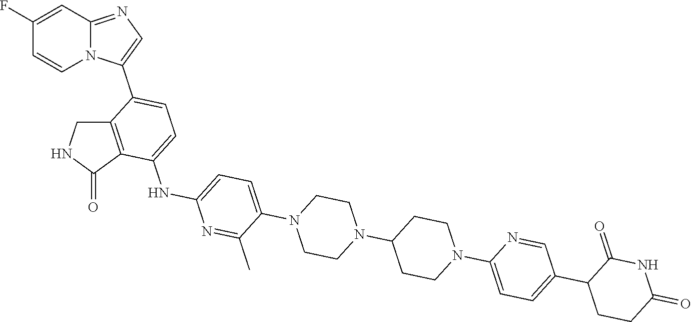 [SMILES: Cc1nc(Nc2ccc(-c3cnc4cc(F)ccn34)c3c2C(=O)NC3)ccc1N1CCN(C2CCN(c3ccc(C4CCC(=O)NC4=O)cn3)CC2)CC1] |
| 180 | 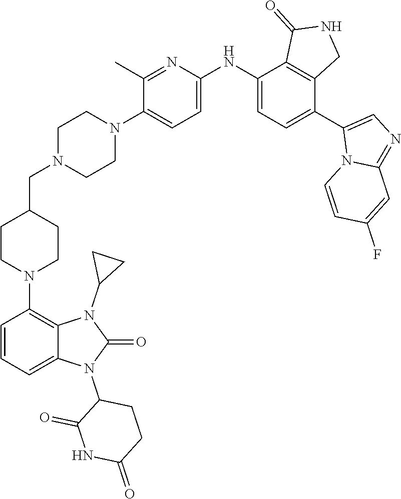 [SMILES: Cc1nc(Nc2ccc(-c3cnc4cc(F)ccn34)c3c2C(=O)NC3)ccc1N1CCN(CC2CCN(c3cccc4c3n(C3CC3)c(=O)n4C3CCC(=O)NC3=O)CC2)CC1] |
| 181 | 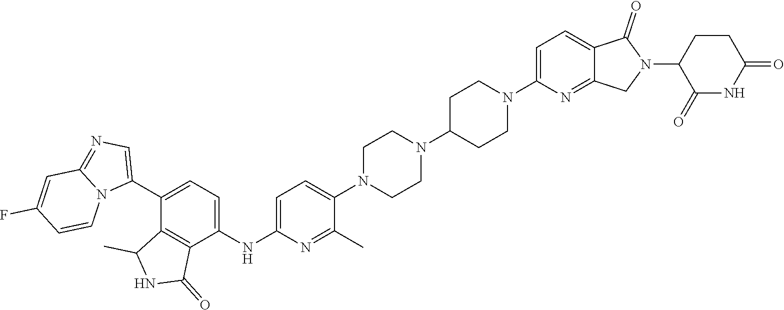 [SMILES: Cc1nc(Nc2ccc(-c3cnc4cc(F)ccn34)c3c2C(=O)NC3C)ccc1N1CCN(C2CCN(c3ccc4c(n3)CN(C3CCC(=O)NC3=O)C4=O)CC2)CC1] |
| 182 | 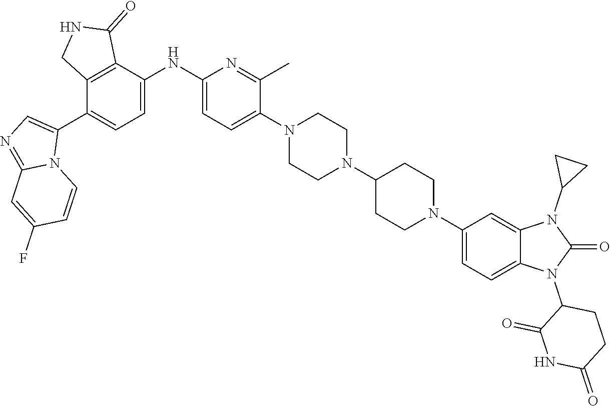 [SMILES: Cc1nc(Nc2ccc(-c3cnc4cc(F)ccn34)c3c2C(=O)NC3)ccc1N1CCN(C2CCN(c3ccc4c(c3)n(C3CC3)c(=O)n4C3CCC(=O)NC3=O)CC2)CC1] |
| 183 | 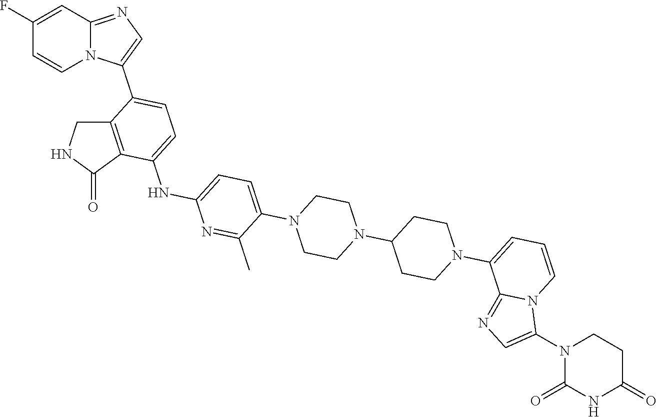 [SMILES: Cc1nc(Nc2ccc(-c3cnc4cc(F)ccn34)c3c2C(=O)NC3)ccc1N1CCN(C2CCN(c3cccn4c(N5CCC(=O)NC5=O)cnc34)CC2)CC1] |
| 184 | 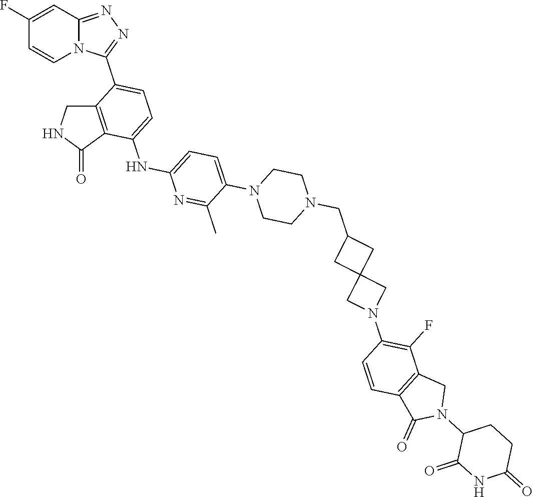 [SMILES: Cc1nc(Nc2ccc(-c3nnc4cc(F)ccn34)c3c2C(=O)NC3)ccc1N1CCN(CC2CC3(C2)CN(c2ccc4c(c2F)CN(C2CCC(=O)NC2=O)C4=O)C3)CC1] |
| 185 | 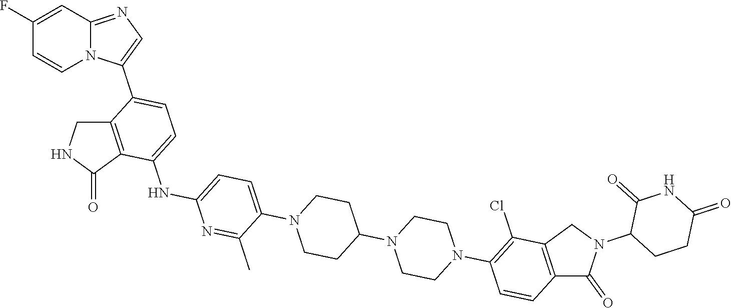 [SMILES: Cc1nc(Nc2ccc(-c3cnc4cc(F)ccn34)c3c2C(=O)NC3)ccc1N1CCC(N2CCN(c3ccc4c(c3Cl)CN(C3CCC(=O)NC3=O)C4=O)CC2)CC1] |
| 186 | 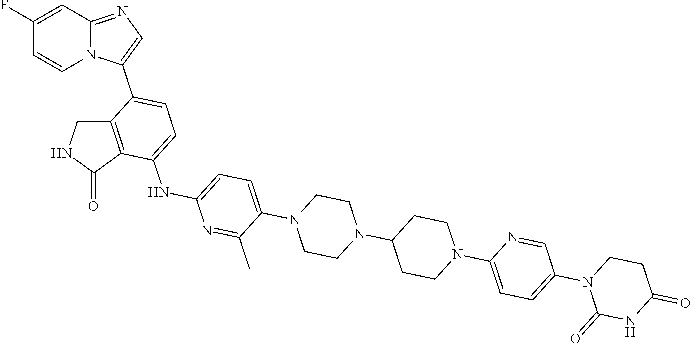 [SMILES: Cc1nc(Nc2ccc(-c3cnc4cc(F)ccn34)c3c2C(=O)NC3)ccc1N1CCN(C2CCN(c3ccc(N4CCC(=O)NC4=O)cn3)CC2)CC1] |
| 187 | 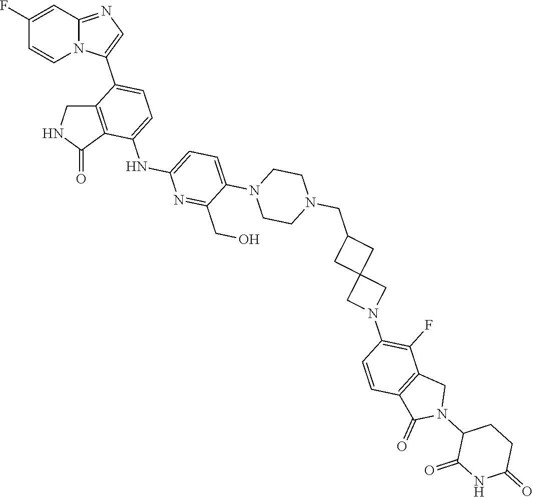 [SMILES: O=C1CCC(N2Cc3c(ccc(N4CC5(CC(CN6CCN(c7ccc(Nc8ccc(-c9cnc%10cc(F)ccn9%10)c9c8C(=O)NC9)nc7CO)CC6)C5)C4)c3F)C2=O)C(=O)N1] |
| 188 | 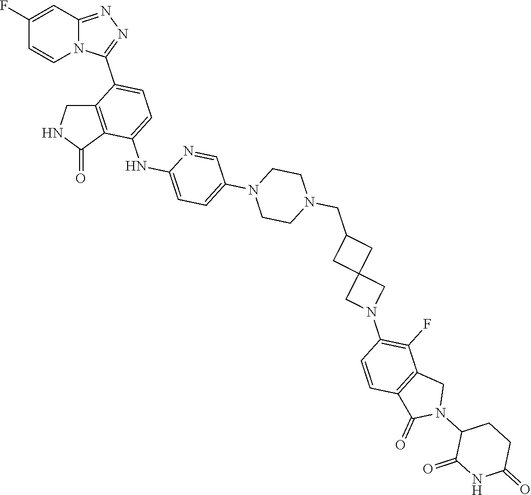 [SMILES: O=C1CCC(N2Cc3c(ccc(N4CC5(CC(CN6CCN(c7ccc(Nc8ccc(-c9nnc%10cc(F)ccn9%10)c9c8C(=O)NC9)nc7)CC6)C5)C4)c3F)C2=O)C(=O)N1] |
| 189 | 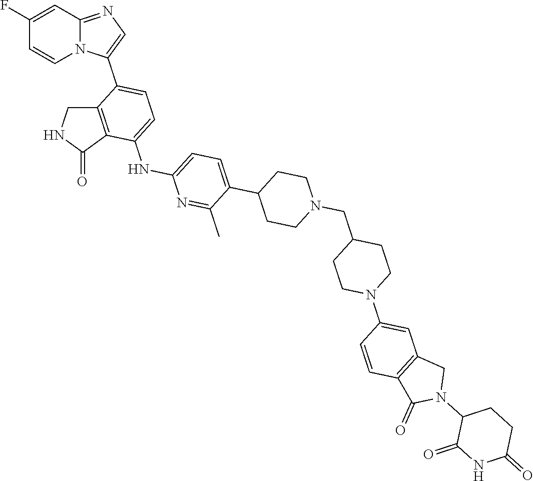 [SMILES: Cc1nc(Nc2ccc(-c3cnc4cc(F)ccn34)c3c2C(=O)NC3)ccc1C1CCN(CC2CCN(c3ccc4c(c3)CN(C3CCC(=O)NC3=O)C4=O)CC2)CC1] |
| 190 | 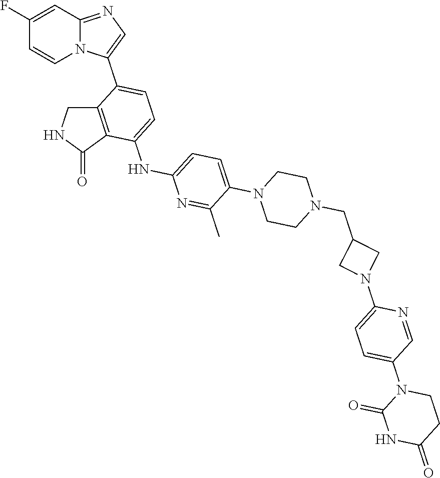 [SMILES: Cc1nc(Nc2ccc(-c3cnc4cc(F)ccn34)c3c2C(=O)NC3)ccc1N1CCN(CC2CN(c3ccc(N4CCC(=O)NC4=O)cn3)C2)CC1] |
| 191 | 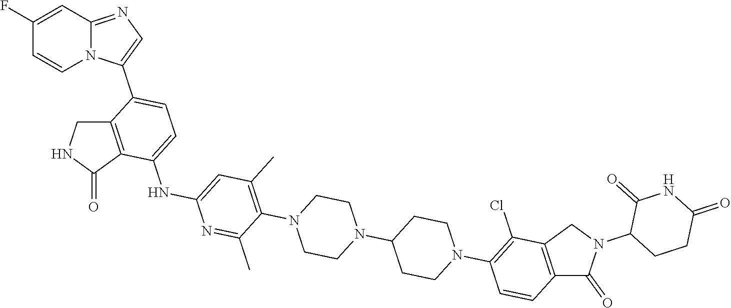 [SMILES: Cc1cc(Nc2ccc(-c3cnc4cc(F)ccn34)c3c2C(=O)NC3)nc(C)c1N1CCN(C2CCN(c3ccc4c(c3Cl)CN(C3CCC(=O)NC3=O)C4=O)CC2)CC1] |
| 192 | 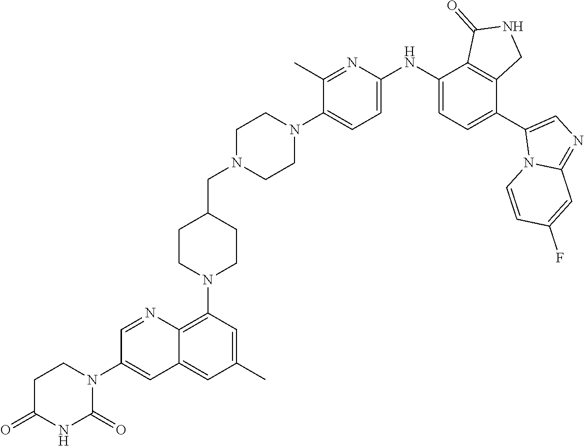 [SMILES: Cc1cc(N2CCC(CN3CCN(c4ccc(Nc5ccc(-c6cnc7cc(F)ccn67)c6c5C(=O)NC6)nc4C)CC3)CC2)c2ncc(N3CCC(=O)NC3=O)cc2c1] |
| 193 | 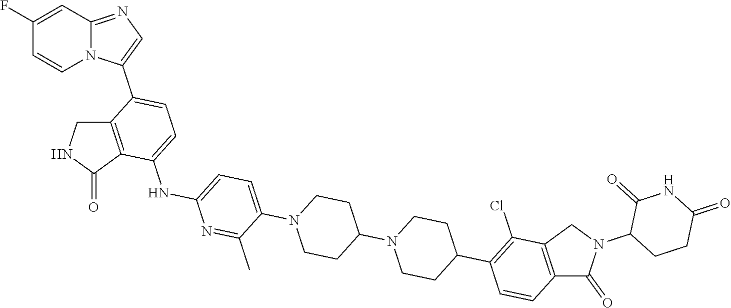 [SMILES: Cc1nc(Nc2ccc(-c3cnc4cc(F)ccn34)c3c2C(=O)NC3)ccc1N1CCC(N2CCC(c3ccc4c(c3Cl)CN(C3CCC(=O)NC3=O)C4=O)CC2)CC1] |
| 194 | 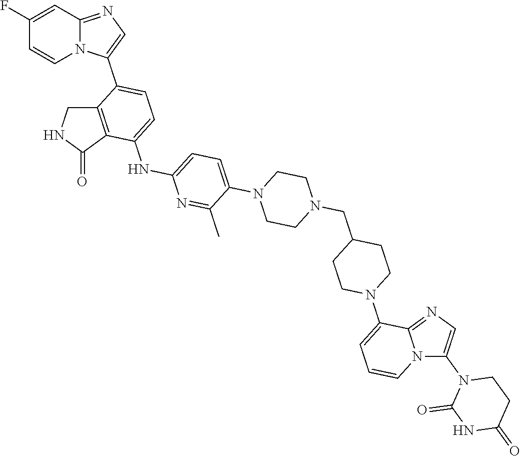 [SMILES: Cc1nc(Nc2ccc(-c3cnc4cc(F)ccn34)c3c2C(=O)NC3)ccc1N1CCN(CC2CCN(c3cccn4c(N5CCC(=O)NC5=O)cnc34)CC2)CC1] |
| 195 | 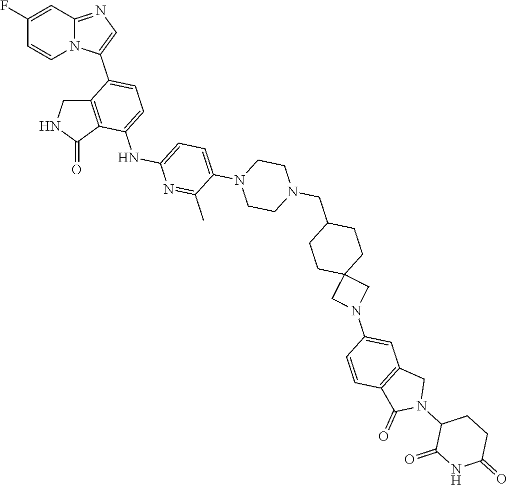 [SMILES: Cc1nc(Nc2ccc(-c3cnc4cc(F)ccn34)c3c2C(=O)NC3)ccc1N1CCN(CC2CCC3(CC2)CN(c2ccc4c(c2)CN(C2CCC(=O)NC2=O)C4=O)C3)CC1] |
| 196 | 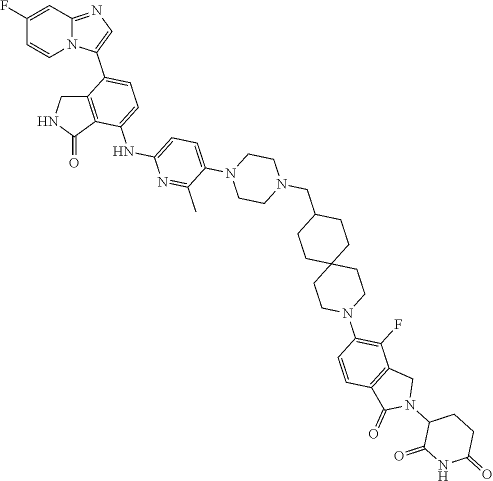 [SMILES: Cc1nc(Nc2ccc(-c3cnc4cc(F)ccn34)c3c2C(=O)NC3)ccc1N1CCN(CC2CCC3(CC2)CCN(c2ccc4c(c2F)CN(C2CCC(=O)NC2=O)C4=O)CC3)CC1] |
| 197 | 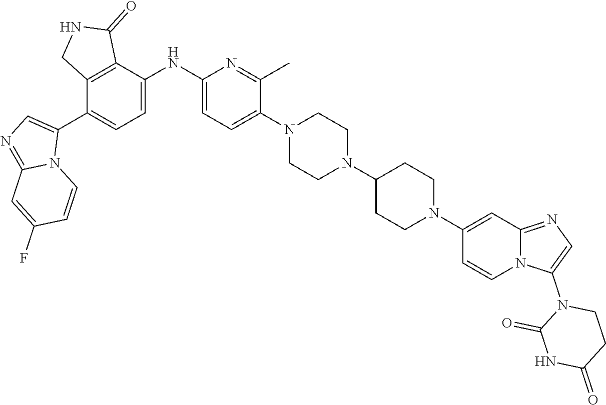 [SMILES: Cc1nc(Nc2ccc(-c3cnc4cc(F)ccn34)c3c2C(=O)NC3)ccc1N1CCN(C2CCN(c3ccn4c(N5CCC(=O)NC5=O)cnc4c3)CC2)CC1] |
| 198 | 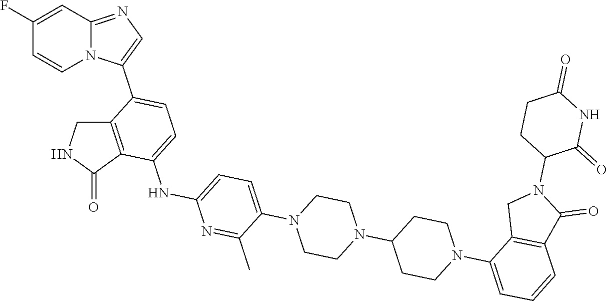 [SMILES: Cc1nc(Nc2ccc(-c3cnc4cc(F)ccn34)c3c2C(=O)NC3)ccc1N1CCN(C2CCN(c3cccc4c3CN(C3CCC(=O)NC3=O)C4=O)CC2)CC1] |
| 199 | 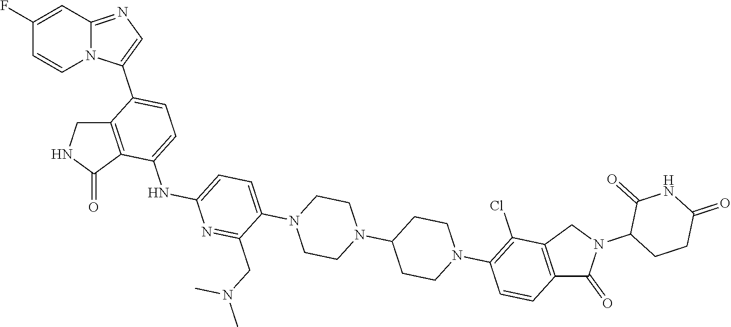 [SMILES: CN(C)Cc1nc(Nc2ccc(-c3cnc4cc(F)ccn34)c3c2C(=O)NC3)ccc1N1CCN(C2CCN(c3ccc4c(c3Cl)CN(C3CCC(=O)NC3=O)C4=O)CC2)CC1] |
| 200 | 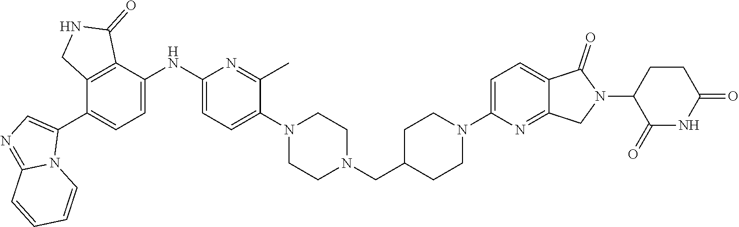 [SMILES: Cc1nc(Nc2ccc(-c3cnc4ccccn34)c3c2C(=O)NC3)ccc1N1CCN(CC2CCN(c3ccc4c(n3)CN(C3CCC(=O)NC3=O)C4=O)CC2)CC1] |
| 201 | 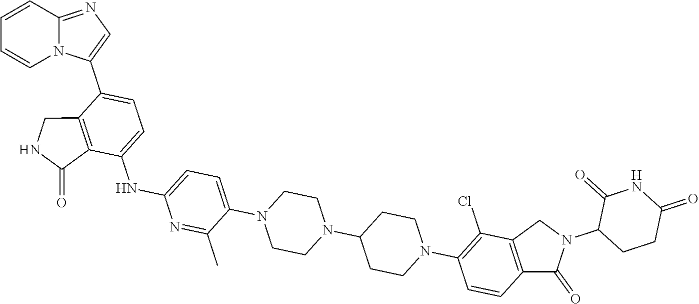 [SMILES: Cc1nc(Nc2ccc(-c3cnc4ccccn34)c3c2C(=O)NC3)ccc1N1CCN(C2CCN(c3ccc4c(c3Cl)CN(C3CCC(=O)NC3=O)C4=O)CC2)CC1] |
| 202 | 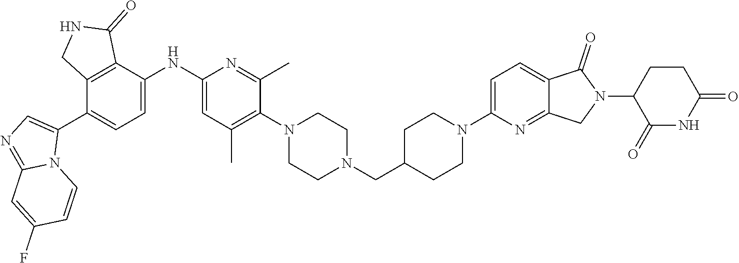 [SMILES: Cc1cc(Nc2ccc(-c3cnc4cc(F)ccn34)c3c2C(=O)NC3)nc(C)c1N1CCN(CC2CCN(c3ccc4c(n3)CN(C3CCC(=O)NC3=O)C4=O)CC2)CC1] |
| 203 | 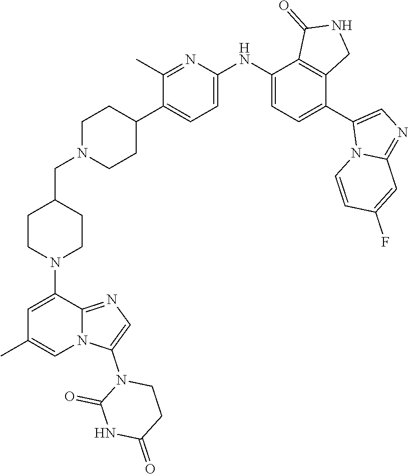 [SMILES: Cc1cc(N2CCC(CN3CCC(c4ccc(Nc5ccc(-c6cnc7cc(F)ccn67)c6c5C(=O)NC6)nc4C)CC3)CC2)c2ncc(N3CCC(=O)NC3=O)n2c1] |
| 204 | 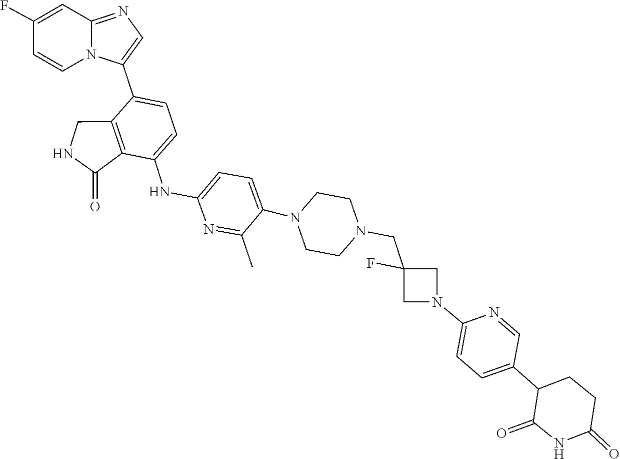 [SMILES: Cc1nc(Nc2ccc(-c3cnc4cc(F)ccn34)c3c2C(=O)NC3)ccc1N1CCN(CC2(F)CN(c3ccc(C4CCC(=O)NC4=O)cn3)C2)CC1] |
| 205 | 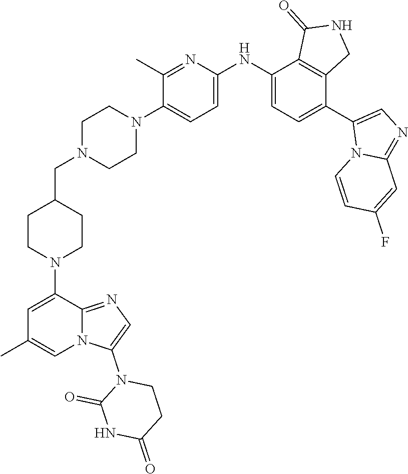 [SMILES: Cc1cc(N2CCC(CN3CCN(c4ccc(Nc5ccc(-c6cnc7cc(F)ccn67)c6c5C(=O)NC6)nc4C)CC3)CC2)c2ncc(N3CCC(=O)NC3=O)n2c1] |
| 206 | 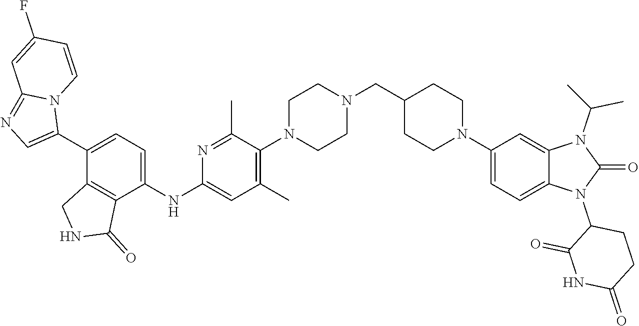 [SMILES: Cc1cc(Nc2ccc(-c3cnc4cc(F)ccn34)c3c2C(=O)NC3)nc(C)c1N1CCN(CC2CCN(c3ccc4c(c3)n(C(C)C)c(=O)n4C3CCC(=O)NC3=O)CC2)CC1] |
| 207 | 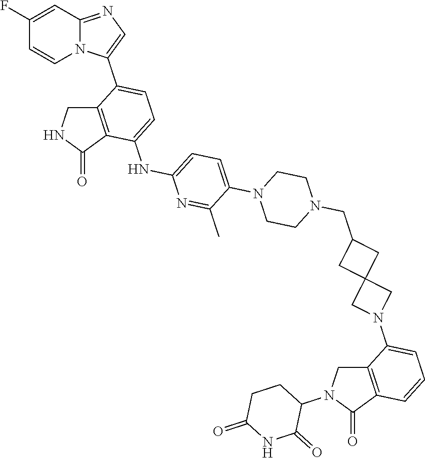 [SMILES: Cc1nc(Nc2ccc(-c3cnc4cc(F)ccn34)c3c2C(=O)NC3)ccc1N1CCN(CC2CC3(C2)CN(c2cccc4c2CN(C2CCC(=O)NC2=O)C4=O)C3)CC1] |
| 208 | 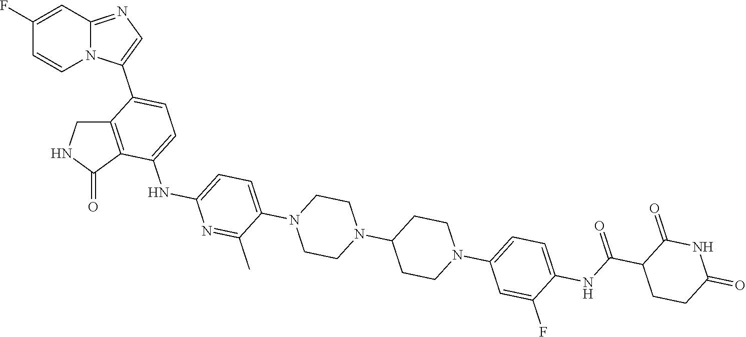 [SMILES: Cc1nc(Nc2ccc(-c3cnc4cc(F)ccn34)c3c2C(=O)NC3)ccc1N1CCN(C2CCN(c3ccc(NC(=O)C4CCC(=O)NC4=O)c(F)c3)CC2)CC1] |
| 209 | 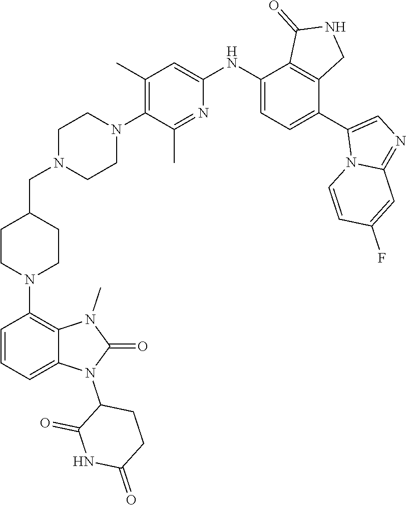 [SMILES: Cc1cc(Nc2ccc(-c3cnc4cc(F)ccn34)c3c2C(=O)NC3)nc(C)c1N1CCN(CC2CCN(c3cccc4c3n(C)c(=O)n4C3CCC(=O)NC3=O)CC2)CC1] |
| 210 | 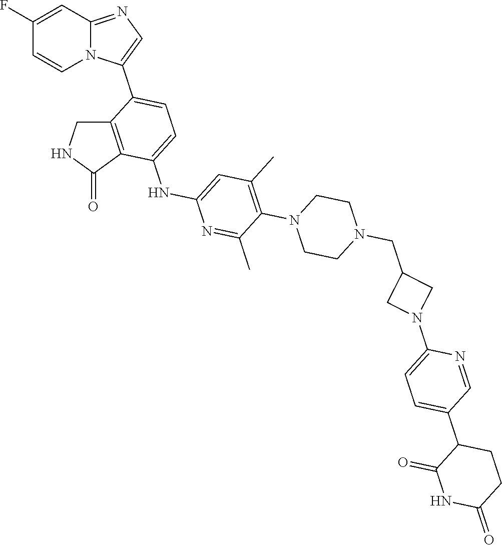 [SMILES: Cc1cc(Nc2ccc(-c3cnc4cc(F)ccn34)c3c2C(=O)NC3)nc(C)c1N1CCN(CC2CN(c3ccc(C4CCC(=O)NC4=O)cn3)C2)CC1] |
| 211 | 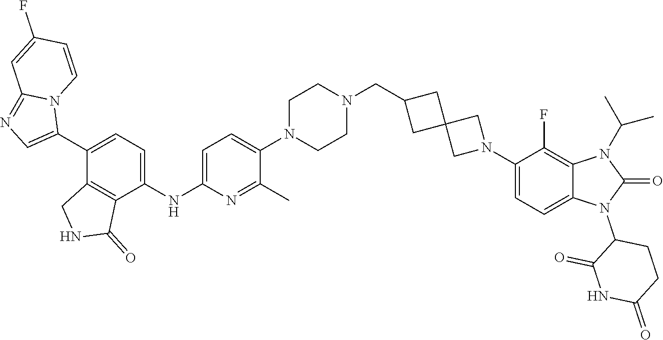 [SMILES: Cc1nc(Nc2ccc(-c3cnc4cc(F)ccn34)c3c2C(=O)NC3)ccc1N1CCN(CC2CC3(C2)CN(c2ccc4c(c2F)n(C(C)C)c(=O)n4C2CCC(=O)NC2=O)C3)CC1] |
| 212 | 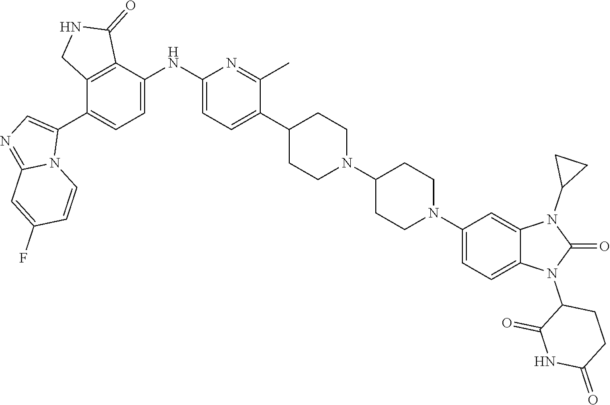 [SMILES: Cc1nc(Nc2ccc(-c3cnc4cc(F)ccn34)c3c2C(=O)NC3)ccc1C1CCN(C2CCN(c3ccc4c(c3)n(C3CC3)c(=O)n4C3CCC(=O)NC3=O)CC2)CC1] |
| 213 | 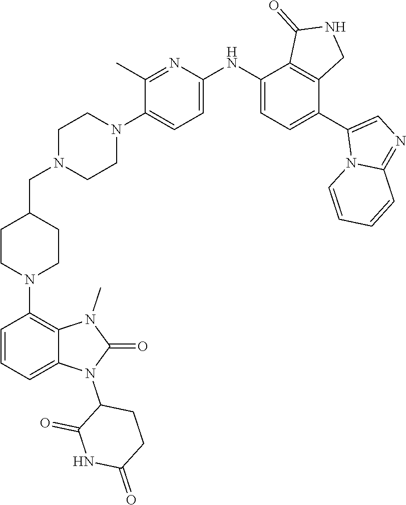 [SMILES: Cc1nc(Nc2ccc(-c3cnc4ccccn34)c3c2C(=O)NC3)ccc1N1CCN(CC2CCN(c3cccc4c3n(C)c(=O)n4C3CCC(=O)NC3=O)CC2)CC1] |
| 214 | 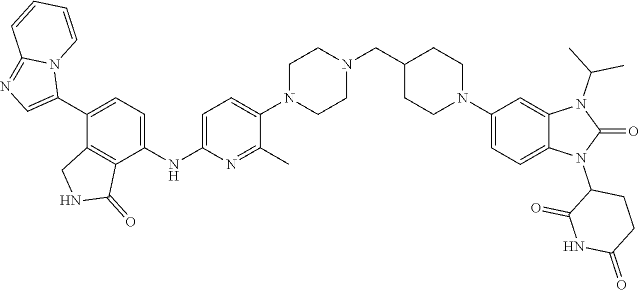 [SMILES: Cc1nc(Nc2ccc(-c3cnc4ccccn34)c3c2C(=O)NC3)ccc1N1CCN(CC2CCN(c3ccc4c(c3)n(C(C)C)c(=O)n4C3CCC(=O)NC3=O)CC2)CC1] |
| 215 | 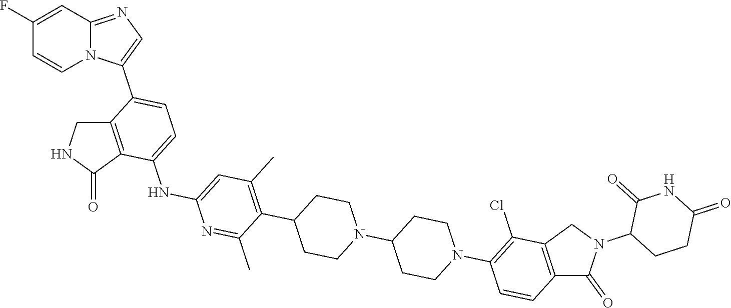 [SMILES: Cc1cc(Nc2ccc(-c3cnc4cc(F)ccn34)c3c2C(=O)NC3)nc(C)c1C1CCN(C2CCN(c3ccc4c(c3Cl)CN(C3CCC(=O)NC3=O)C4=O)CC2)CC1] |
| 216 | 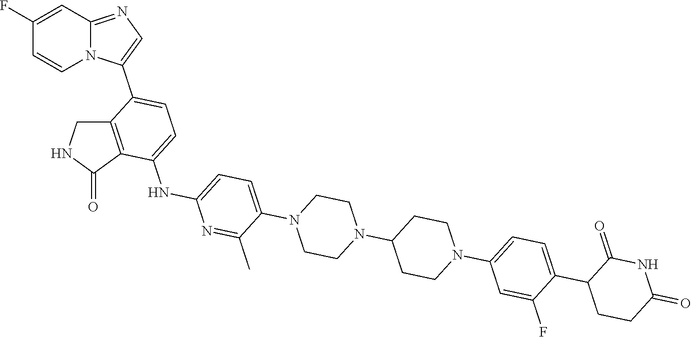 [SMILES: Cc1nc(Nc2ccc(-c3cnc4cc(F)ccn34)c3c2C(=O)NC3)ccc1N1CCN(C2CCN(c3ccc(C4CCC(=O)NC4=O)c(F)c3)CC2)CC1] |
| 217 | 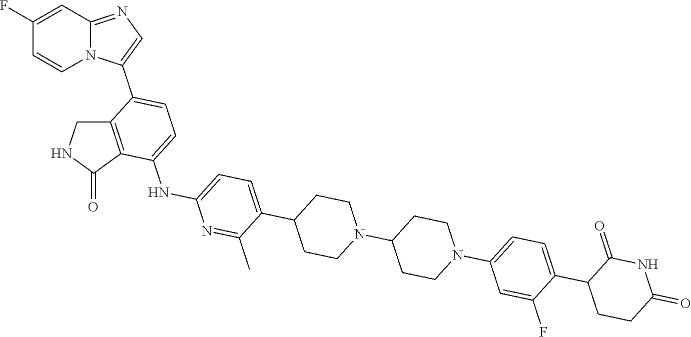 [SMILES: Cc1nc(Nc2ccc(-c3cnc4cc(F)ccn34)c3c2C(=O)NC3)ccc1C1CCN(C2CCN(c3ccc(C4CCC(=O)NC4=O)c(F)c3)CC2)CC1] |
| 218 | 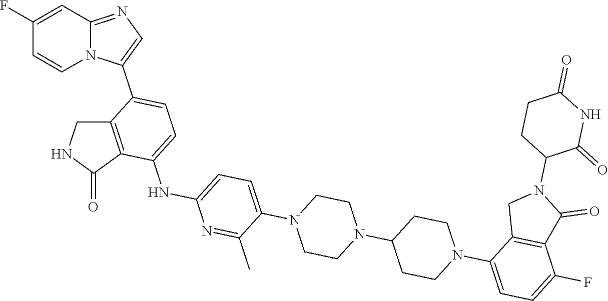 [SMILES: Cc1nc(Nc2ccc(-c3cnc4cc(F)ccn34)c3c2C(=O)NC3)ccc1N1CCN(C2CCN(c3ccc(F)c4c3CN(C3CCC(=O)NC3=O)C4=O)CC2)CC1] |
| 219 | 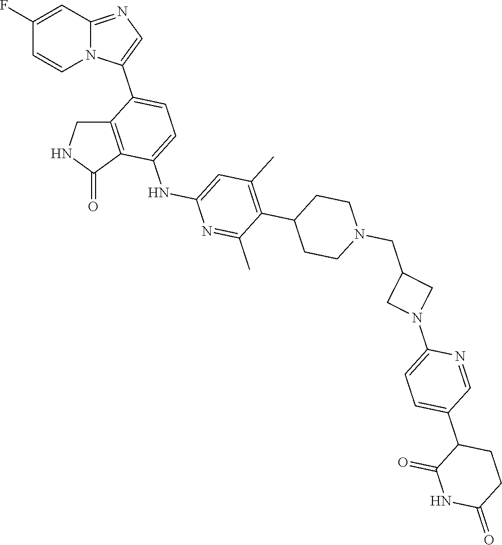 [SMILES: Cc1cc(Nc2ccc(-c3cnc4cc(F)ccn34)c3c2C(=O)NC3)nc(C)c1C1CCN(CC2CN(c3ccc(C4CCC(=O)NC4=O)cn3)C2)CC1] |
| 220 | 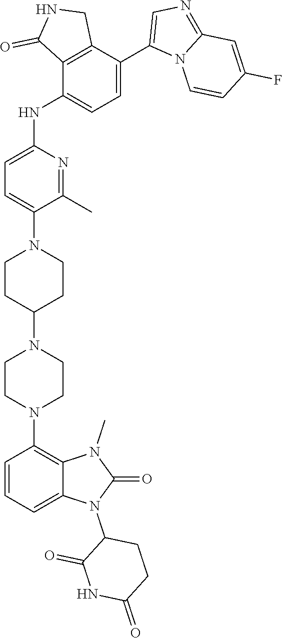 [SMILES: Cc1nc(Nc2ccc(-c3cnc4cc(F)ccn34)c3c2C(=O)NC3)ccc1N1CCC(N2CCN(c3cccc4c3n(C)c(=O)n4C3CCC(=O)NC3=O)CC2)CC1] |
| 221 | 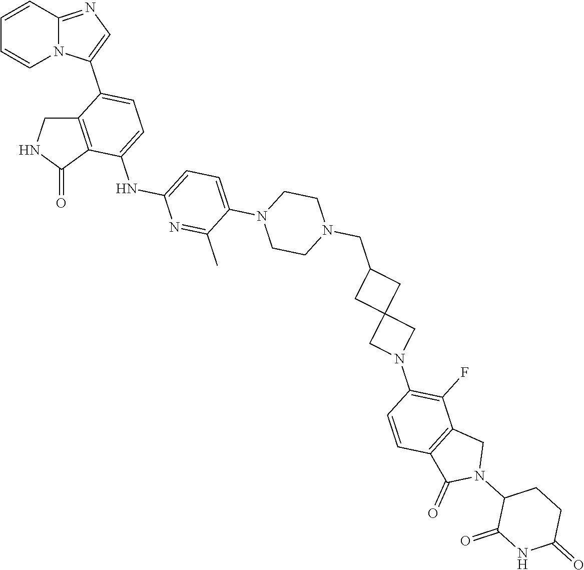 [SMILES: Cc1nc(Nc2ccc(-c3cnc4ccccn34)c3c2C(=O)NC3)ccc1N1CCN(CC2CC3(C2)CN(c2ccc4c(c2F)CN(C2CCC(=O)NC2=O)C4=O)C3)CC1] |
| 222 | 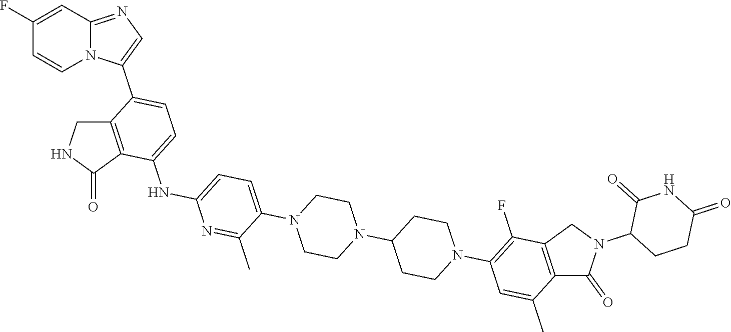 [SMILES: Cc1cc(N2CCC(N3CCN(c4ccc(Nc5ccc(-c6cnc7cc(F)ccn67)c6c5C(=O)NC6)nc4C)CC3)CC2)c(F)c2c1C(=O)N(C1CCC(=O)NC1=O)C2] |
| 223 | 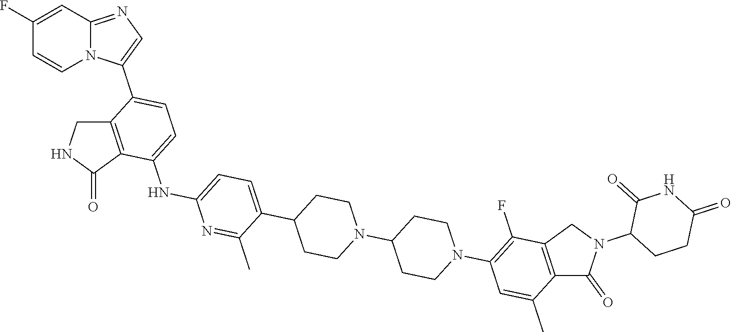 [SMILES: Cc1cc(N2CCC(N3CCC(c4ccc(Nc5ccc(-c6cnc7cc(F)ccn67)c6c5C(=O)NC6)nc4C)CC3)CC2)c(F)c2c1C(=O)N(C1CCC(=O)NC1=O)C2] |
| 224 | 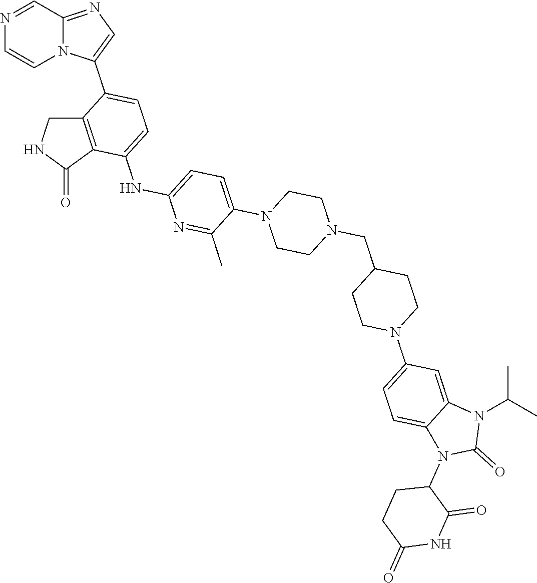 [SMILES: Cc1nc(Nc2ccc(-c3cnc4cnccn34)c3c2C(=O)NC3)ccc1N1CCN(CC2CCN(c3ccc4c(c3)n(C(C)C)c(=O)n4C3CCC(=O)NC3=O)CC2)CC1] |
| 225 | 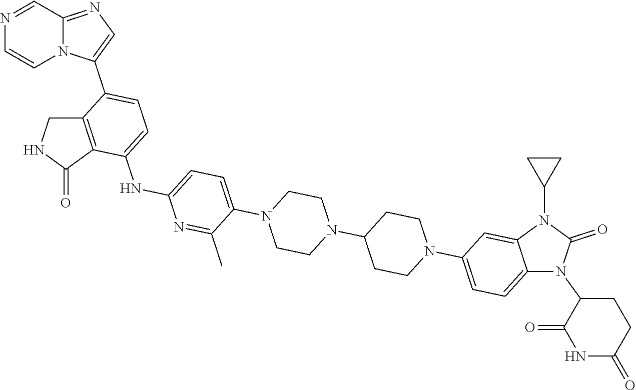 [SMILES: Cc1nc(Nc2ccc(-c3cnc4cnccn34)c3c2C(=O)NC3)ccc1N1CCN(C2CCN(c3ccc4c(c3)n(C3CC3)c(=O)n4C3CCC(=O)NC3=O)CC2)CC1] |
| 226 | 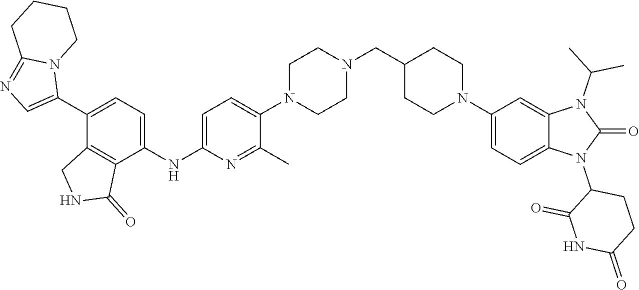 [SMILES: Cc1nc(Nc2ccc(-c3cnc4n3CCCC4)c3c2C(=O)NC3)ccc1N1CCN(CC2CCN(c3ccc4c(c3)n(C(C)C)c(=O)n4C3CCC(=O)NC3=O)CC2)CC1] |
| 227 | 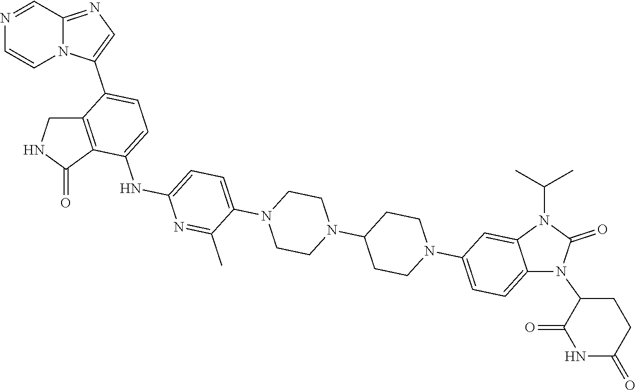 [SMILES: Cc1nc(Nc2ccc(-c3cnc4cnccn34)c3c2C(=O)NC3)ccc1N1CCN(C2CCN(c3ccc4c(c3)n(C(C)C)c(=O)n4C3CCC(=O)NC3=O)CC2)CC1] |
| 228 | 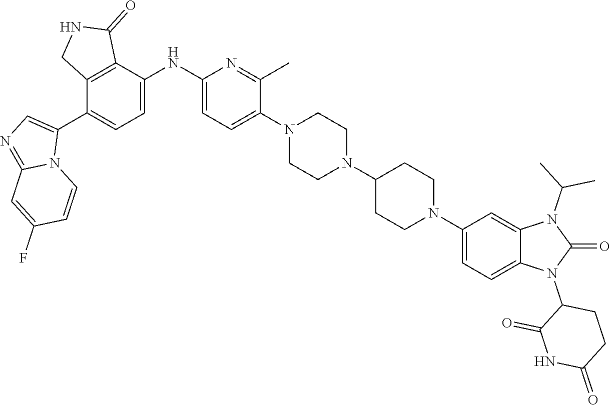 [SMILES: Cc1nc(Nc2ccc(-c3cnc4cc(F)ccn34)c3c2C(=O)NC3)ccc1N1CCN(C2CCN(c3ccc4c(c3)n(C(C)C)c(=O)n4C3CCC(=O)NC3=O)CC2)CC1] |
| 229 | 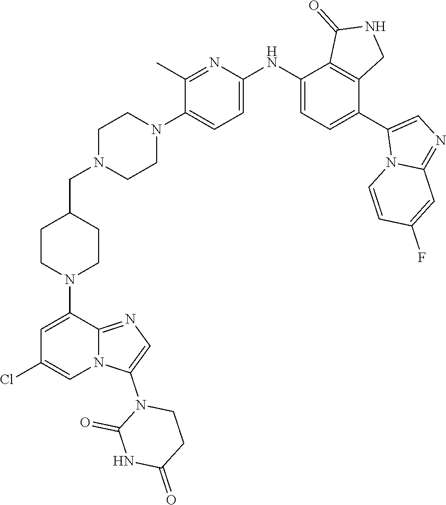 [SMILES: Cc1nc(Nc2ccc(-c3cnc4cc(F)ccn34)c3c2C(=O)NC3)ccc1N1CCN(CC2CCN(c3cc(Cl)cn4c(N5CCC(=O)NC5=O)cnc34)CC2)CC1] |
| 230 | 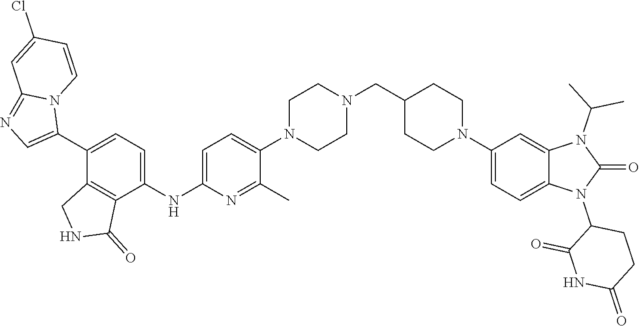 [SMILES: Cc1nc(Nc2ccc(-c3cnc4cc(Cl)ccn34)c3c2C(=O)NC3)ccc1N1CCN(CC2CCN(c3ccc4c(c3)n(C(C)C)c(=O)n4C3CCC(=O)NC3=O)CC2)CC1] |
| 231 | 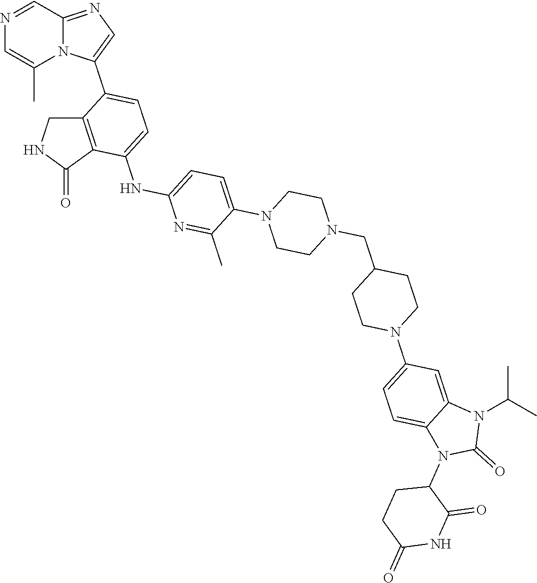 [SMILES: Cc1nc(Nc2ccc(-c3cnc4cncc(C)n34)c3c2C(=O)NC3)ccc1N1CCN(CC2CCN(c3ccc4c(c3)n(C(C)C)c(=O)n4C3CCC(=O)NC3=O)CC2)CC1] |
| 232 | 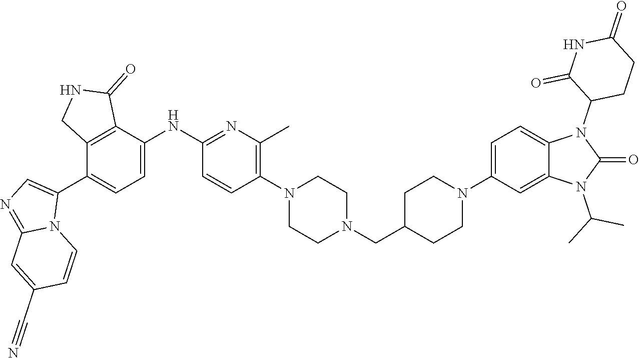 [SMILES: Cc1nc(Nc2ccc(-c3cnc4cc(C#N)ccn34)c3c2C(=O)NC3)ccc1N1CCN(CC2CCN(c3ccc4c(c3)n(C(C)C)c(=O)n4C3CCC(=O)NC3=O)CC2)CC1] |
| 233 | 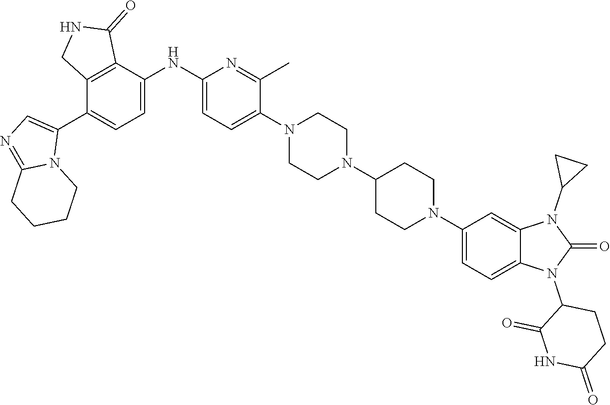 [SMILES: Cc1nc(Nc2ccc(-c3cnc4n3CCCC4)c3c2C(=O)NC3)ccc1N1CCN(C2CCN(c3ccc4c(c3)n(C3CC3)c(=O)n4C3CCC(=O)NC3=O)CC2)CC1] |
| 234 | 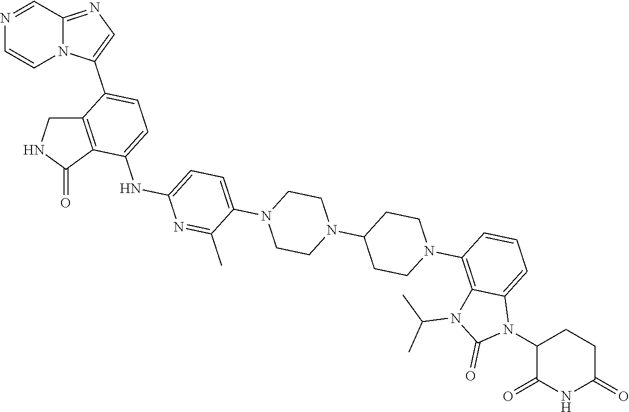 [SMILES: Cc1nc(Nc2ccc(-c3cnc4cnccn34)c3c2C(=O)NC3)ccc1N1CCN(C2CCN(c3cccc4c3n(C(C)C)c(=O)n4C3CCC(=O)NC3=O)CC2)CC1] |
| 235 | 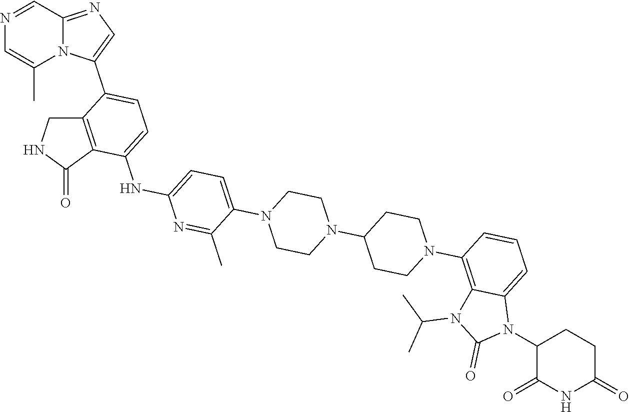 [SMILES: Cc1nc(Nc2ccc(-c3cnc4cncc(C)n34)c3c2C(=O)NC3)ccc1N1CCN(C2CCN(c3cccc4c3n(C(C)C)c(=O)n4C3CCC(=O)NC3=O)CC2)CC1] |
| 236 | 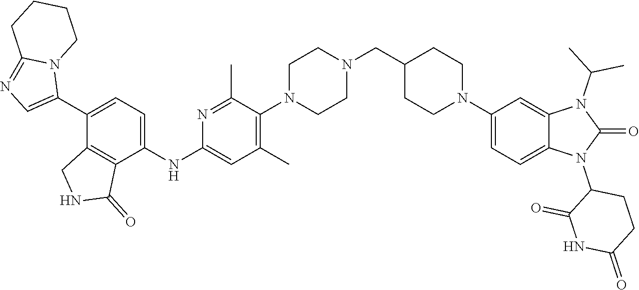 [SMILES: Cc1cc(Nc2ccc(-c3cnc4n3CCCC4)c3c2C(=O)NC3)nc(C)c1N1CCN(CC2CCN(c3ccc4c(c3)n(C(C)C)c(=O)n4C3CCC(=O)NC3=O)CC2)CC1] |
| 237 | 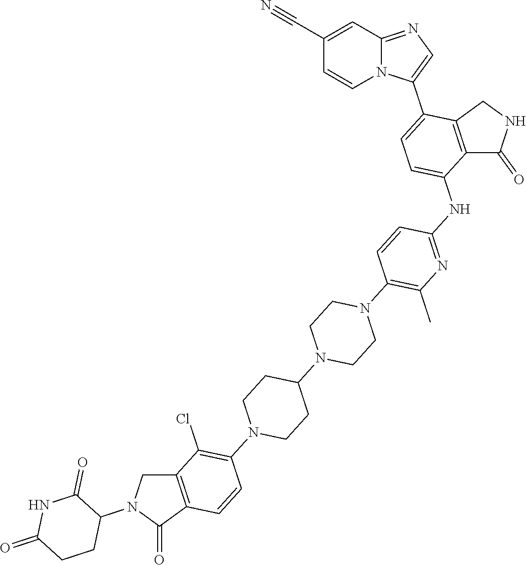 [SMILES: Cc1nc(Nc2ccc(-c3cnc4cc(C#N)ccn34)c3c2C(=O)NC3)ccc1N1CCN(C2CCN(c3ccc4c(c3Cl)CN(C3CCC(=O)NC3=O)C4=O)CC2)CC1] |
| 238 | 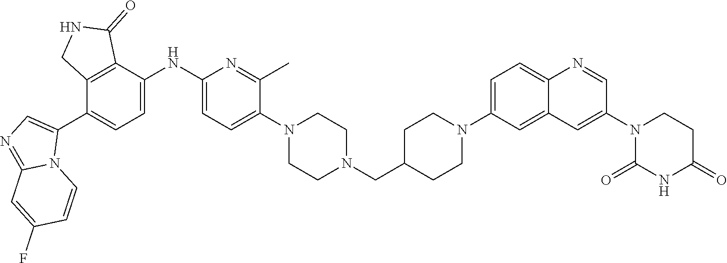 [SMILES: Cc1nc(Nc2ccc(-c3cnc4cc(F)ccn34)c3c2C(=O)NC3)ccc1N1CCN(CC2CCN(c3ccc4ncc(N5CCC(=O)NC5=O)cc4c3)CC2)CC1] |
| 239 | 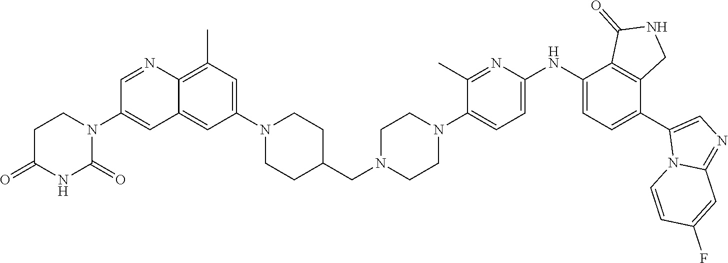 [SMILES: Cc1nc(Nc2ccc(-c3cnc4cc(F)ccn34)c3c2C(=O)NC3)ccc1N1CCN(CC2CCN(c3cc(C)c4ncc(N5CCC(=O)NC5=O)cc4c3)CC2)CC1] |
| 240 | 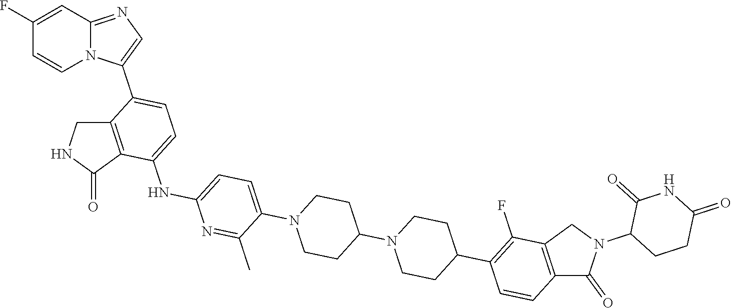 [SMILES: Cc1nc(Nc2ccc(-c3cnc4cc(F)ccn34)c3c2C(=O)NC3)ccc1N1CCC(N2CCC(c3ccc4c(c3F)CN(C3CCC(=O)NC3=O)C4=O)CC2)CC1] |
| 241 | 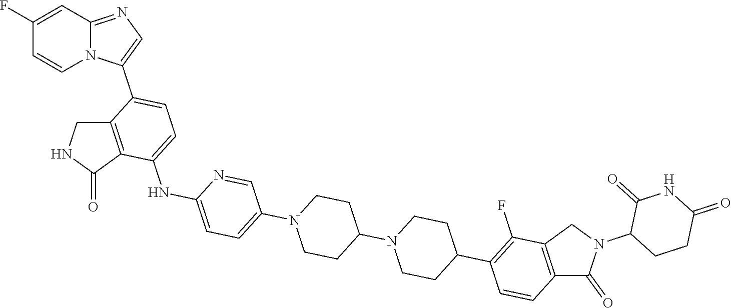 [SMILES: O=C1CCC(N2Cc3c(ccc(C4CCN(C5CCN(c6ccc(Nc7ccc(-c8cnc9cc(F)ccn89)c8c7C(=O)NC8)nc6)CC5)CC4)c3F)C2=O)C(=O)N1] |
| 242 | 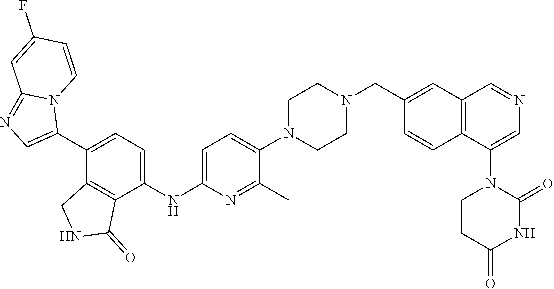 [SMILES: Cc1nc(Nc2ccc(-c3cnc4cc(F)ccn34)c3c2C(=O)NC3)ccc1N1CCN(Cc2ccc3c(N4CCC(=O)NC4=O)cncc3c2)CC1] |
| 243 | 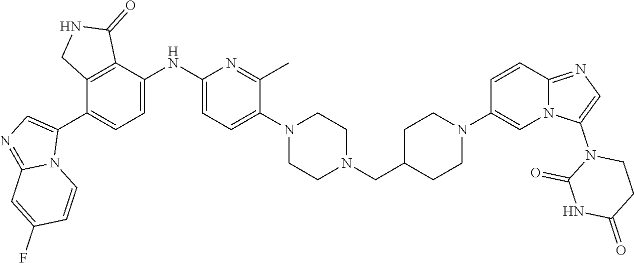 [SMILES: Cc1nc(Nc2ccc(-c3cnc4cc(F)ccn34)c3c2C(=O)NC3)ccc1N1CCN(CC2CCN(c3ccc4ncc(N5CCC(=O)NC5=O)n4c3)CC2)CC1] |
| 244 | 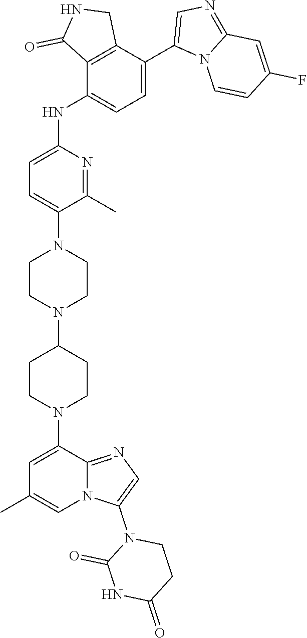 [SMILES: Cc1cc(N2CCC(N3CCN(c4ccc(Nc5ccc(-c6cnc7cc(F)ccn67)c6c5C(=O)NC6)nc4C)CC3)CC2)c2ncc(N3CCC(=O)NC3=O)n2c1] |
| 245 | 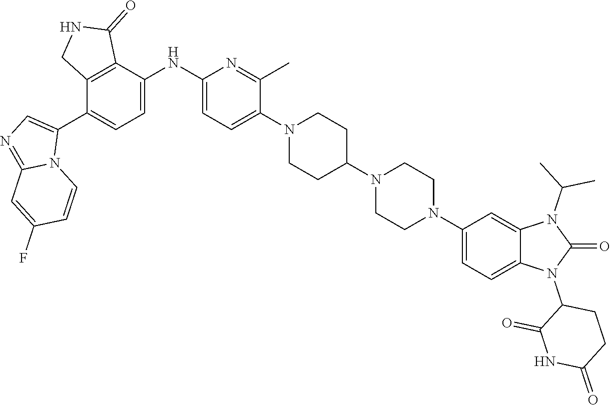 [SMILES: Cc1nc(Nc2ccc(-c3cnc4cc(F)ccn34)c3c2C(=O)NC3)ccc1N1CCC(N2CCN(c3ccc4c(c3)n(C(C)C)c(=O)n4C3CCC(=O)NC3=O)CC2)CC1] |
| 246 | 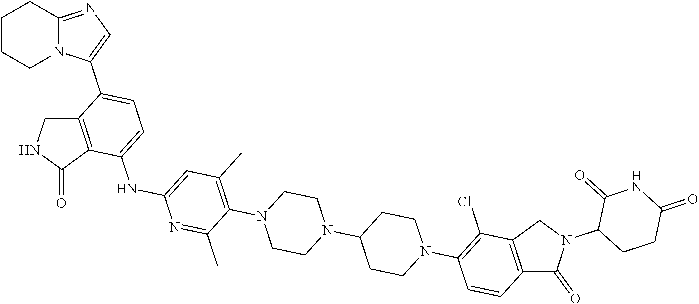 [SMILES: Cc1cc(Nc2ccc(-c3cnc4n3CCCC4)c3c2C(=O)NC3)nc(C)c1N1CCN(C2CCN(c3ccc4c(c3Cl)CN(C3CCC(=O)NC3=O)C4=O)CC2)CC1] |
| 247 | 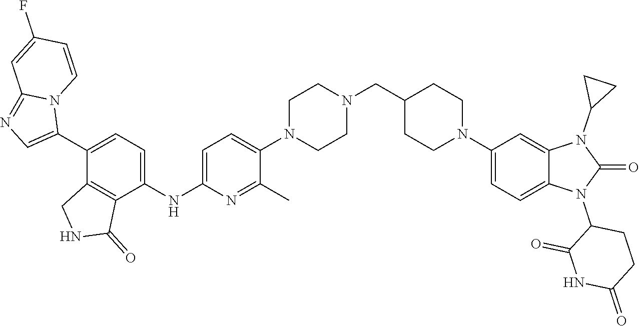 [SMILES: Cc1nc(Nc2ccc(-c3cnc4cc(F)ccn34)c3c2C(=O)NC3)ccc1N1CCN(CC2CCN(c3ccc4c(c3)n(C3CC3)c(=O)n4C3CCC(=O)NC3=O)CC2)CC1] |
| 248 | 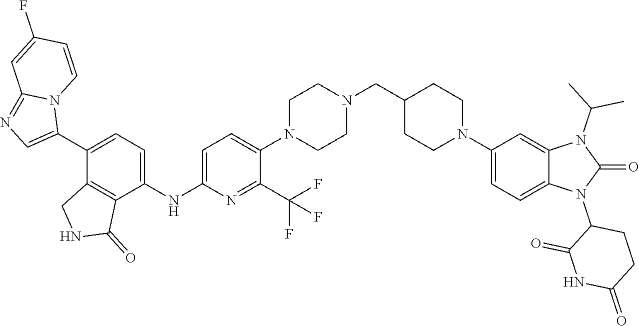 [SMILES: CC(C)n1c(=O)n(C2CCC(=O)NC2=O)c2ccc(N3CCC(CN4CCN(c5ccc(Nc6ccc(-c7cnc8cc(F)ccn78)c7c6C(=O)NC7)nc5C(F)(F)F)CC4)CC3)cc21] |
| 249 | 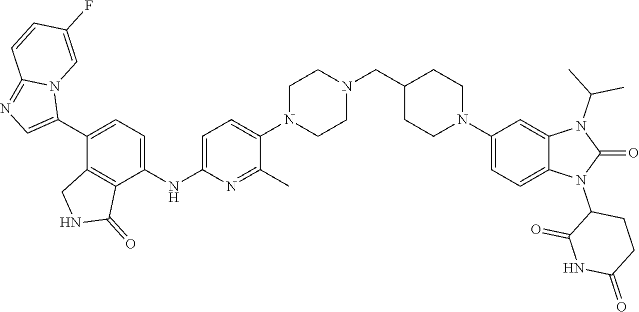 [SMILES: Cc1nc(Nc2ccc(-c3cnc4ccc(F)cn34)c3c2C(=O)NC3)ccc1N1CCN(CC2CCN(c3ccc4c(c3)n(C(C)C)c(=O)n4C3CCC(=O)NC3=O)CC2)CC1] |
| 250 | 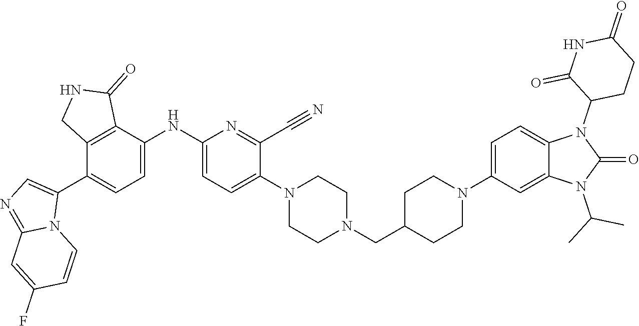 [SMILES: CC(C)n1c(=O)n(C2CCC(=O)NC2=O)c2ccc(N3CCC(CN4CCN(c5ccc(Nc6ccc(-c7cnc8cc(F)ccn78)c7c6C(=O)NC7)nc5C#N)CC4)CC3)cc21] |
| 251 | 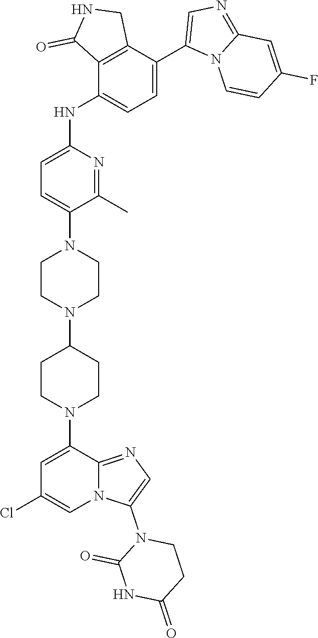 [SMILES: Cc1nc(Nc2ccc(-c3cnc4cc(F)ccn34)c3c2C(=O)NC3)ccc1N1CCN(C2CCN(c3cc(Cl)cn4c(N5CCC(=O)NC5=O)cnc34)CC2)CC1] |
| 252 | 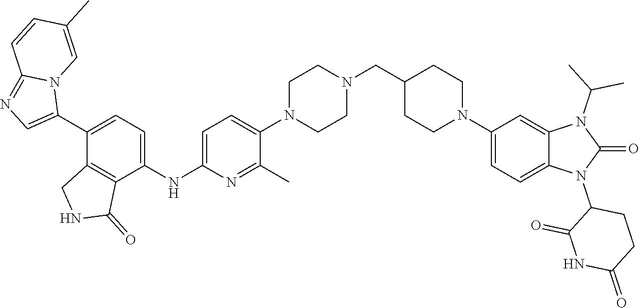 [SMILES: Cc1ccc2ncc(-c3ccc(Nc4ccc(N5CCN(CC6CCN(c7ccc8c(c7)n(C(C)C)c(=O)n8C7CCC(=O)NC7=O)CC6)CC5)c(C)n4)c4c3CNC4=O)n2c1] |
| 253 | 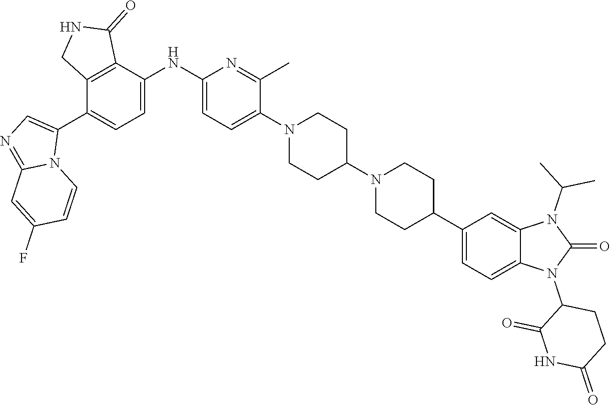 [SMILES: Cc1nc(Nc2ccc(-c3cnc4cc(F)ccn34)c3c2C(=O)NC3)ccc1N1CCC(N2CCC(c3ccc4c(c3)n(C(C)C)c(=O)n4C3CCC(=O)NC3=O)CC2)CC1] |
| 254 | 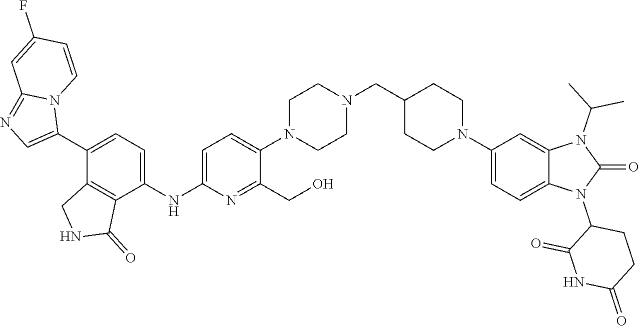 [SMILES: CC(C)n1c(=O)n(C2CCC(=O)NC2=O)c2ccc(N3CCC(CN4CCN(c5ccc(Nc6ccc(-c7cnc8cc(F)ccn78)c7c6C(=O)NC7)nc5CO)CC4)CC3)cc21] |
| 255 | 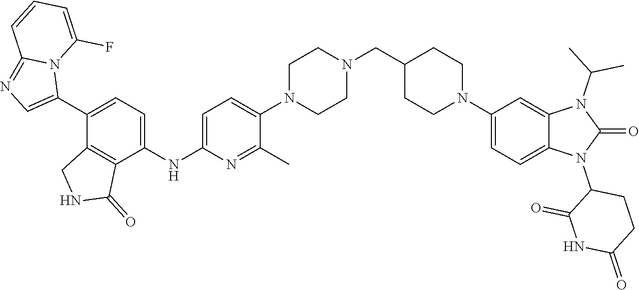 [SMILES: Cc1nc(Nc2ccc(-c3cnc4cccc(F)n34)c3c2C(=O)NC3)ccc1N1CCN(CC2CCN(c3ccc4c(c3)n(C(C)C)c(=O)n4C3CCC(=O)NC3=O)CC2)CC1] |
| 256 | 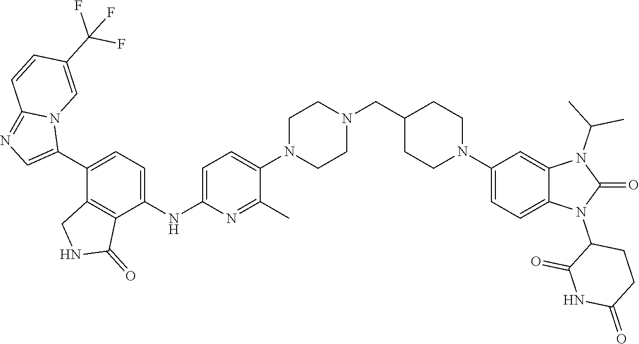 [SMILES: Cc1nc(Nc2ccc(-c3cnc4ccc(C(F)(F)F)cn34)c3c2C(=O)NC3)ccc1N1CCN(CC2CCN(c3ccc4c(c3)n(C(C)C)c(=O)n4C3CCC(=O)NC3=O)CC2)CC1] |
| 257 | 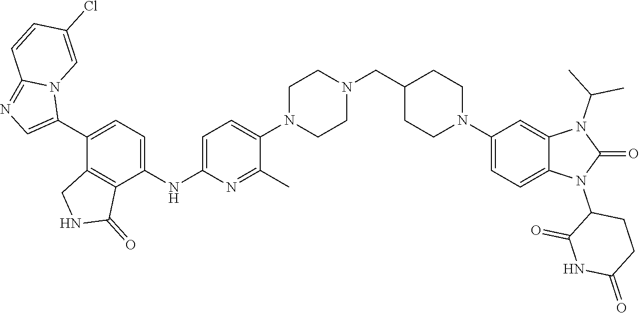 [SMILES: Cc1nc(Nc2ccc(-c3cnc4ccc(Cl)cn34)c3c2C(=O)NC3)ccc1N1CCN(CC2CCN(c3ccc4c(c3)n(C(C)C)c(=O)n4C3CCC(=O)NC3=O)CC2)CC1] |
| 258 | 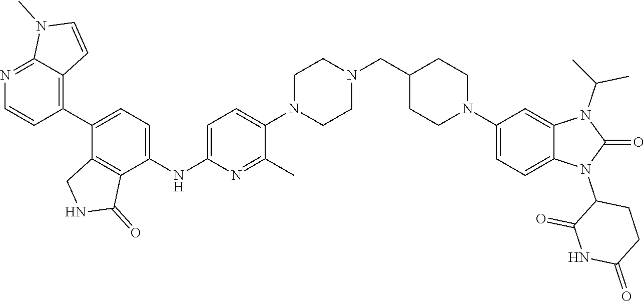 [SMILES: Cc1nc(Nc2ccc(-c3ccnc4c3ccn4C)c3c2C(=O)NC3)ccc1N1CCN(CC2CCN(c3ccc4c(c3)n(C(C)C)c(=O)n4C3CCC(=O)NC3=O)CC2)CC1] |
| 259 | 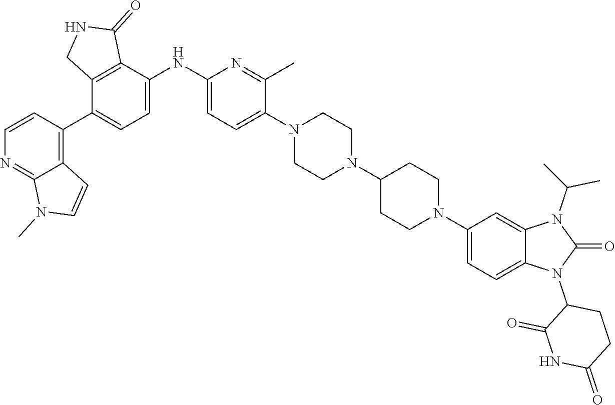 [SMILES: Cc1nc(Nc2ccc(-c3ccnc4c3ccn4C)c3c2C(=O)NC3)ccc1N1CCN(C2CCN(c3ccc4c(c3)n(C(C)C)c(=O)n4C3CCC(=O)NC3=O)CC2)CC1] |
| 260 | 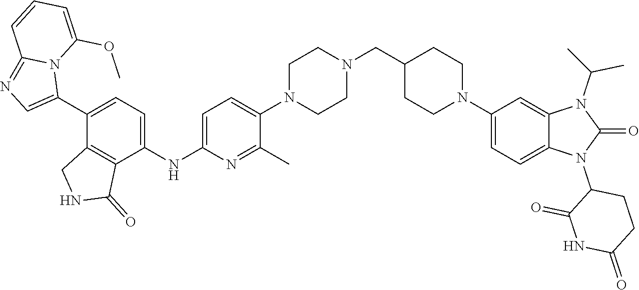 [SMILES: COc1cccc2ncc(-c3ccc(Nc4ccc(N5CCN(CC6CCN(c7ccc8c(c7)n(C(C)C)c(=O)n8C7CCC(=O)NC7=O)CC6)CC5)c(C)n4)c4c3CNC4=O)n12] |
| 261 | 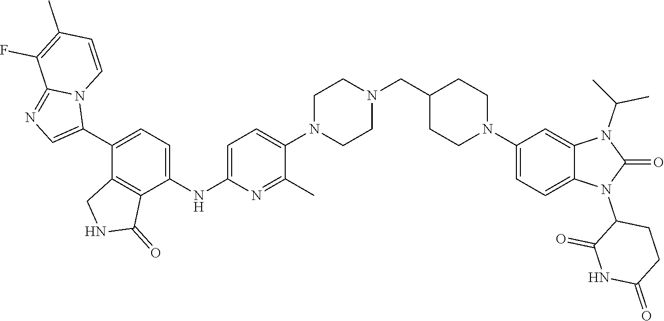 [SMILES: Cc1ccn2c(-c3ccc(Nc4ccc(N5CCN(CC6CCN(c7ccc8c(c7)n(C(C)C)c(=O)n8C7CCC(=O)NC7=O)CC6)CC5)c(C)n4)c4c3CNC4=O)cnc2c1F] |
| 262 | 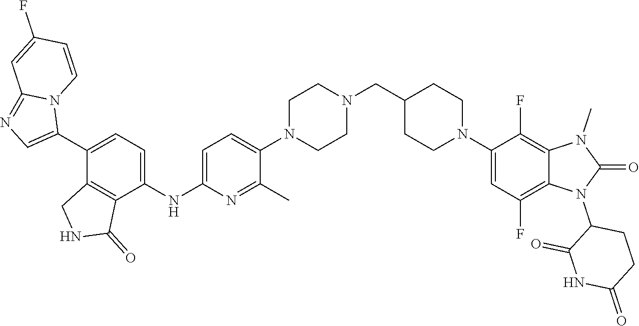 [SMILES: Cc1nc(Nc2ccc(-c3cnc4cc(F)ccn34)c3c2C(=O)NC3)ccc1N1CCN(CC2CCN(c3cc(F)c4c(c3F)n(C)c(=O)n4C3CCC(=O)NC3=O)CC2)CC1] |
| 263 | 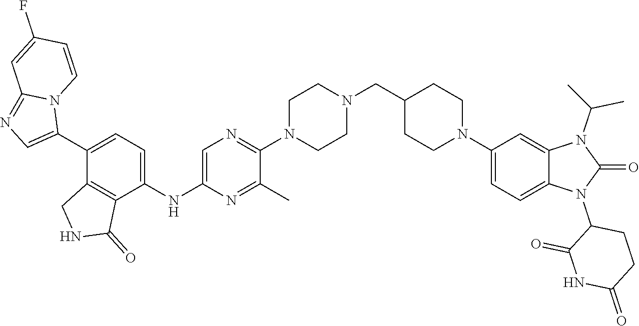 [SMILES: Cc1nc(Nc2ccc(-c3cnc4cc(F)ccn34)c3c2C(=O)NC3)cnc1N1CCN(CC2CCN(c3ccc4c(c3)n(C(C)C)c(=O)n4C3CCC(=O)NC3=O)CC2)CC1] |
| 264 | 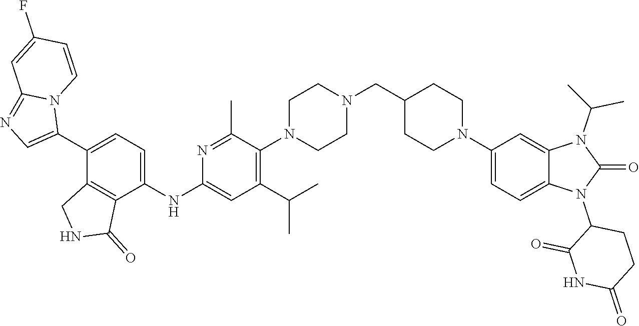 [SMILES: Cc1nc(Nc2ccc(-c3cnc4cc(F)ccn34)c3c2C(=O)NC3)cc(C(C)C)c1N1CCN(CC2CCN(c3ccc4c(c3)n(C(C)C)c(=O)n4C3CCC(=O)NC3=O)CC2)CC1] |
| 265 | 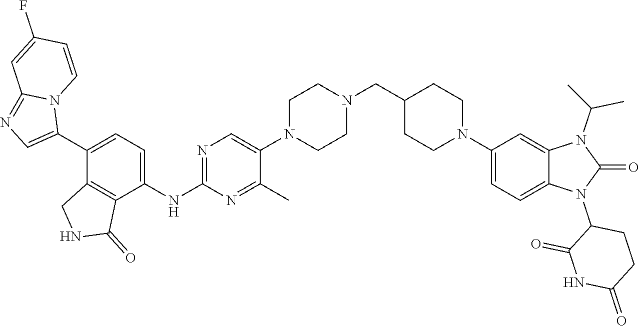 [SMILES: Cc1nc(Nc2ccc(-c3cnc4cc(F)ccn34)c3c2C(=O)NC3)ncc1N1CCN(CC2CCN(c3ccc4c(c3)n(C(C)C)c(=O)n4C3CCC(=O)NC3=O)CC2)CC1] |
| 266 | 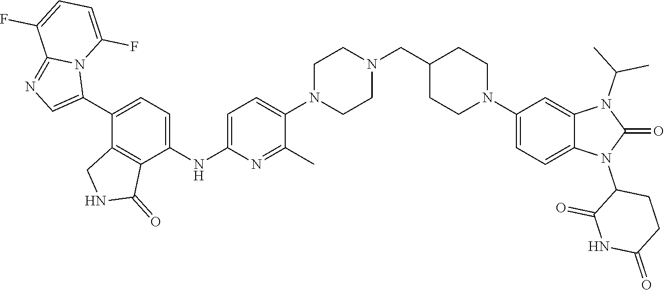 [SMILES: Cc1nc(Nc2ccc(-c3cnc4c(F)ccc(F)n34)c3c2C(=O)NC3)ccc1N1CCN(CC2CCN(c3ccc4c(c3)n(C(C)C)c(=O)n4C3CCC(=O)NC3=O)CC2)CC1] |
| 267 | 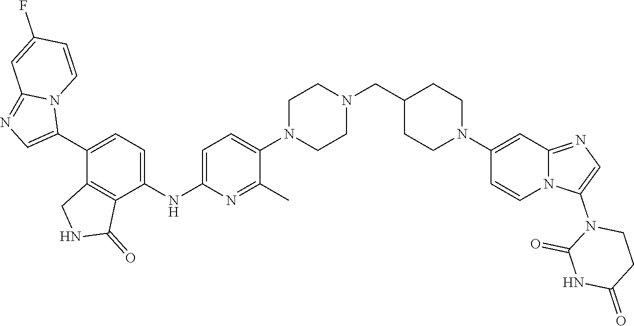 [SMILES: Cc1nc(Nc2ccc(-c3cnc4cc(F)ccn34)c3c2C(=O)NC3)ccc1N1CCN(CC2CCN(c3ccn4c(N5CCC(=O)NC5=O)cnc4c3)CC2)CC1] |
| 268 | 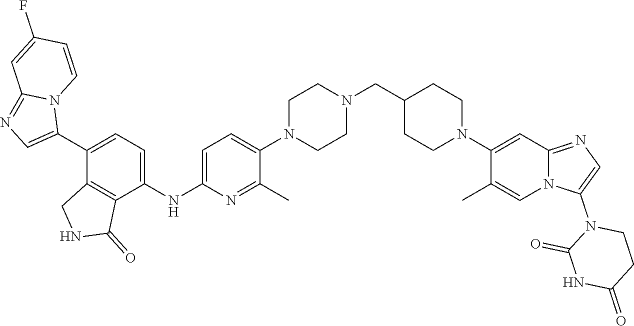 [SMILES: Cc1cn2c(N3CCC(=O)NC3=O)cnc2cc1N1CCC(CN2CCN(c3ccc(Nc4ccc(-c5cnc6cc(F)ccn56)c5c4C(=O)NC5)nc3C)CC2)CC1] |
| 269 | 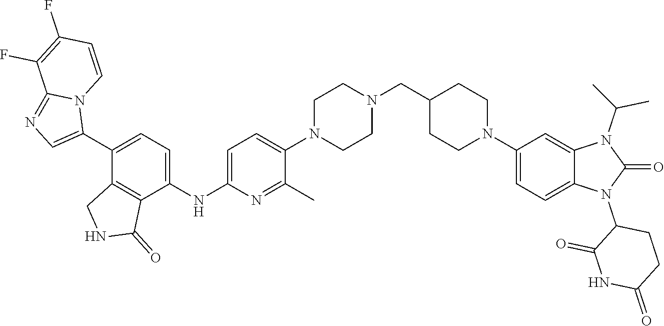 [SMILES: Cc1nc(Nc2ccc(-c3cnc4c(F)c(F)ccn34)c3c2C(=O)NC3)ccc1N1CCN(CC2CCN(c3ccc4c(c3)n(C(C)C)c(=O)n4C3CCC(=O)NC3=O)CC2)CC1] |
| 270 | 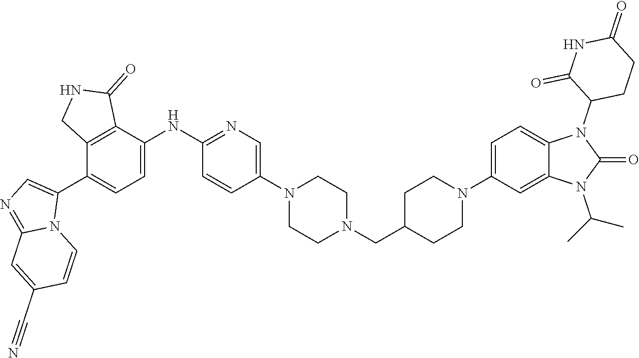 [SMILES: CC(C)n1c(=O)n(C2CCC(=O)NC2=O)c2ccc(N3CCC(CN4CCN(c5ccc(Nc6ccc(-c7cnc8cc(C#N)ccn78)c7c6C(=O)NC7)nc5)CC4)CC3)cc21] |
| 271 | 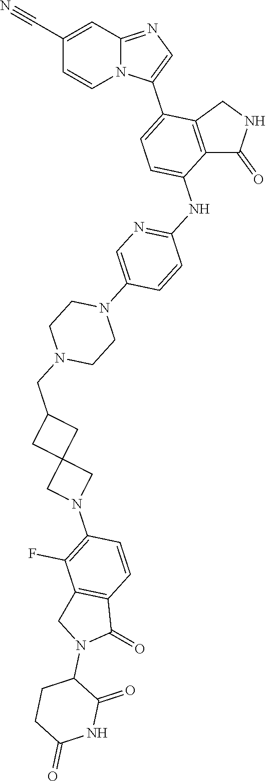 [SMILES: N#Cc1ccn2c(-c3ccc(Nc4ccc(N5CCN(CC6CC7(C6)CN(c6ccc8c(c6F)CN(C6CCC(=O)NC6=O)C8=O)C7)CC5)cn4)c4c3CNC4=O)cnc2c1] |
| 272 | 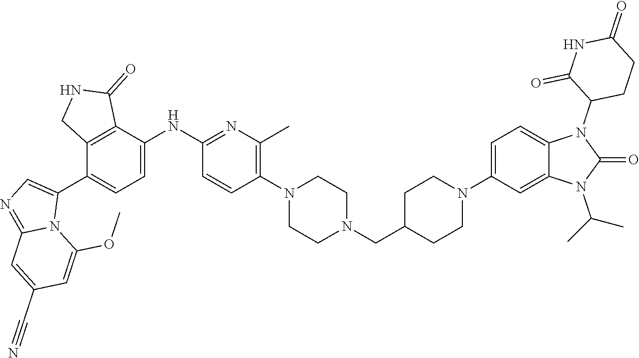 [SMILES: COc1cc(C#N)cc2ncc(-c3ccc(Nc4ccc(N5CCN(CC6CCN(c7ccc8c(c7)n(C(C)C)c(=O)n8C7CCC(=O)NC7=O)CC6)CC5)c(C)n4)c4c3CNC4=O)n12] |
| 273 | 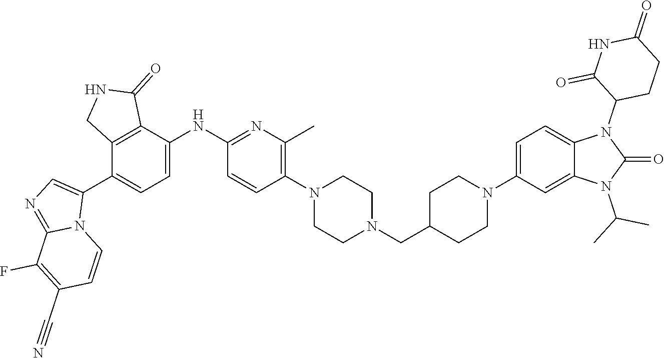 [SMILES: Cc1nc(Nc2ccc(-c3cnc4c(F)c(C#N)ccn34)c3c2C(=O)NC3)ccc1N1CCN(CC2CCN(c3ccc4c(c3)n(C(C)C)c(=O)n4C3CCC(=O)NC3=O)CC2)CC1] |
| 274 | 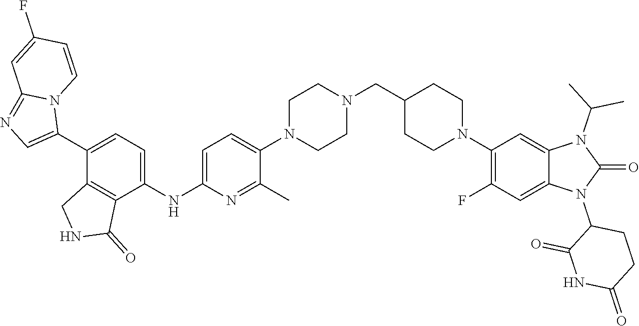 [SMILES: Cc1nc(Nc2ccc(-c3cnc4cc(F)ccn34)c3c2C(=O)NC3)ccc1N1CCN(CC2CCN(c3cc4c(cc3F)n(C3CCC(=O)NC3=O)c(=O)n4C(C)C)CC2)CC1] |
| 275 | 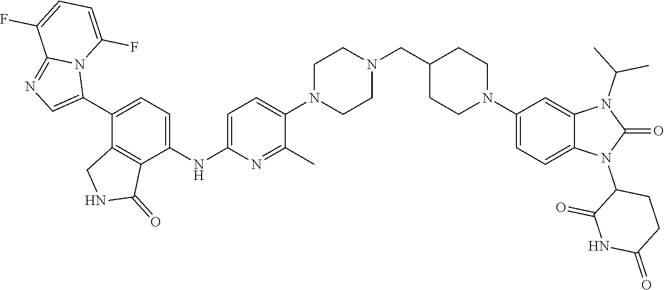 [SMILES: Cc1nc(Nc2ccc(-c3cnc4c(F)ccc(F)n34)c3c2C(=O)NC3)ccc1N1CCN(CC2CCN(c3ccc4c(c3)n(C(C)C)c(=O)n4C3CCC(=O)NC3=O)CC2)CC1] |
| 276 | 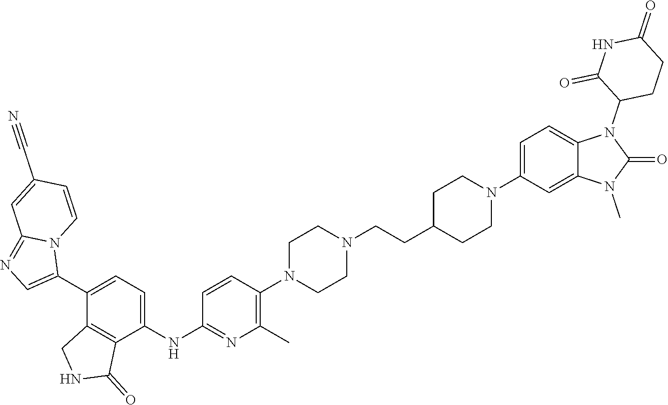 [SMILES: Cc1nc(Nc2ccc(-c3cnc4cc(C#N)ccn34)c3c2C(=O)NC3)ccc1N1CCN(CCC2CCN(c3ccc4c(c3)n(C)c(=O)n4C3CCC(=O)NC3=O)CC2)CC1] |
| 277 | 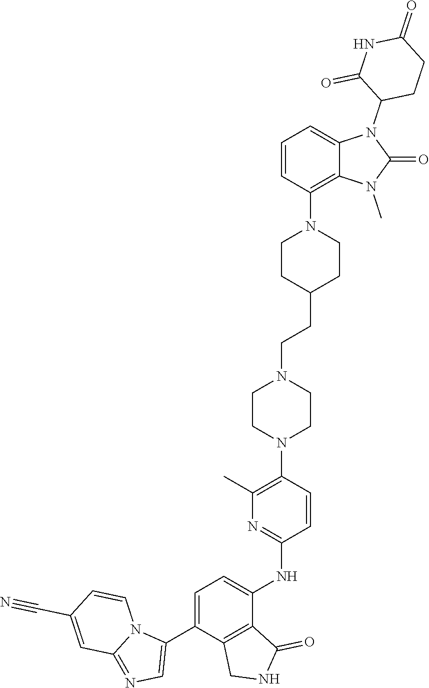 [SMILES: Cc1nc(Nc2ccc(-c3cnc4cc(C#N)ccn34)c3c2C(=O)NC3)ccc1N1CCN(CCC2CCN(c3cccc4c3n(C)c(=O)n4C3CCC(=O)NC3=O)CC2)CC1] |
| 278 | 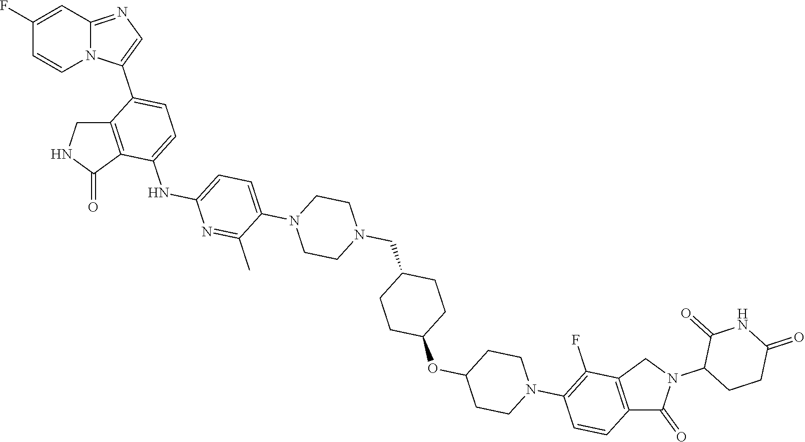 [SMILES: Cc1nc(Nc2ccc(-c3cnc4cc(F)ccn34)c3c2C(=O)NC3)ccc1N1CCN(C[C@H]2CC[C@H](OC3CCN(c4ccc5c(c4F)CN(C4CCC(=O)NC4=O)C5=O)CC3)CC2)CC1] |
| 279 | 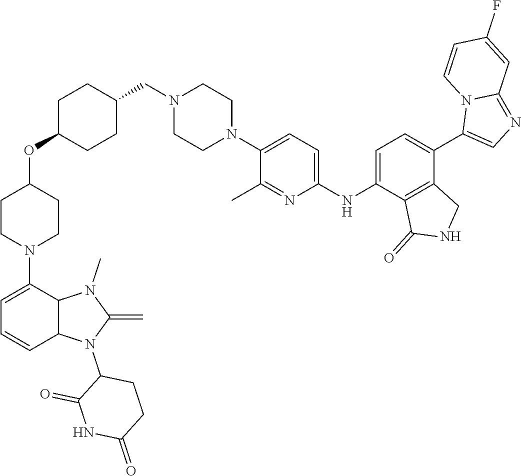 [SMILES: C=C1N(C)C2C(N3CCC(O[C@H]4CC[C@H](CN5CCN(c6ccc(Nc7ccc(-c8cnc9cc(F)ccn89)c8c7C(=O)NC8)nc6C)CC5)CC4)CC3)=CC=CC2N1C1CCC(=O)NC1=O] |
| 280 | 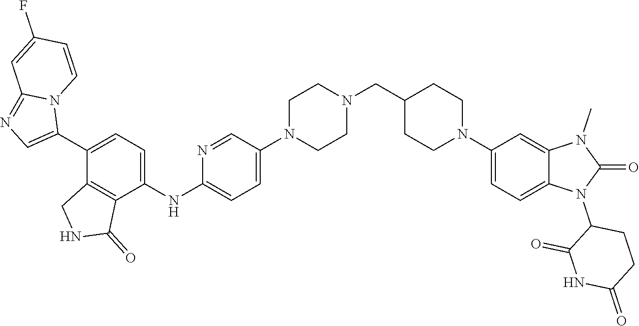 [SMILES: Cn1c(=O)n(C2CCC(=O)NC2=O)c2ccc(N3CCC(CN4CCN(c5ccc(Nc6ccc(-c7cnc8cc(F)ccn78)c7c6C(=O)NC7)nc5)CC4)CC3)cc21] |
| 281 | 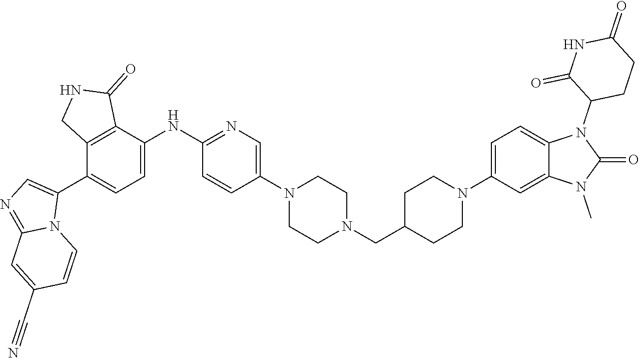 [SMILES: Cn1c(=O)n(C2CCC(=O)NC2=O)c2ccc(N3CCC(CN4CCN(c5ccc(Nc6ccc(-c7cnc8cc(C#N)ccn78)c7c6C(=O)NC7)nc5)CC4)CC3)cc21] |
| 282 | 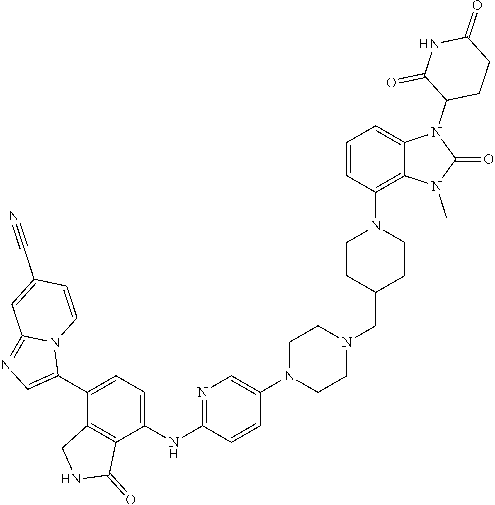 [SMILES: Cn1c(=O)n(C2CCC(=O)NC2=O)c2cccc(N3CCC(CN4CCN(c5ccc(Nc6ccc(-c7cnc8cc(C#N)ccn78)c7c6C(=O)NC7)nc5)CC4)CC3)c21] |
| 283 | 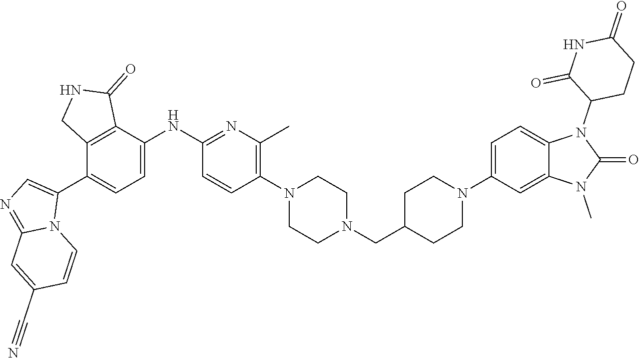 [SMILES: Cc1nc(Nc2ccc(-c3cnc4cc(C#N)ccn34)c3c2C(=O)NC3)ccc1N1CCN(CC2CCN(c3ccc4c(c3)n(C)c(=O)n4C3CCC(=O)NC3=O)CC2)CC1] |
| 284 | 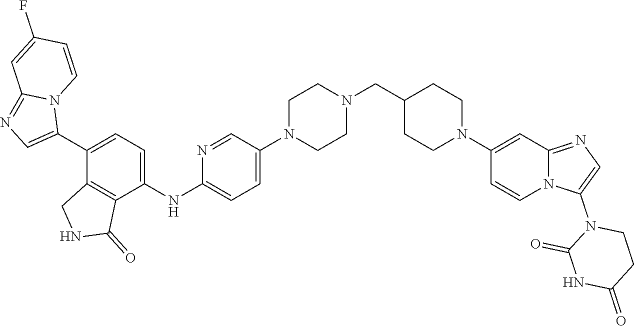 [SMILES: O=C1CCN(c2cnc3cc(N4CCC(CN5CCN(c6ccc(Nc7ccc(-c8cnc9cc(F)ccn89)c8c7C(=O)NC8)nc6)CC5)CC4)ccn23)C(=O)N1] |
| 285 | 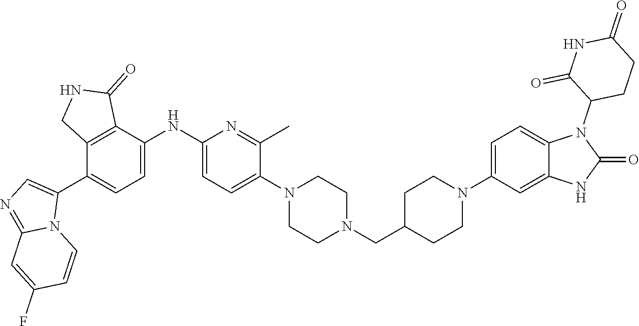 [SMILES: Cc1nc(Nc2ccc(-c3cnc4cc(F)ccn34)c3c2C(=O)NC3)ccc1N1CCN(CC2CCN(c3ccc4c(c3)[nH]c(=O)n4C3CCC(=O)NC3=O)CC2)CC1] |
| 286 | 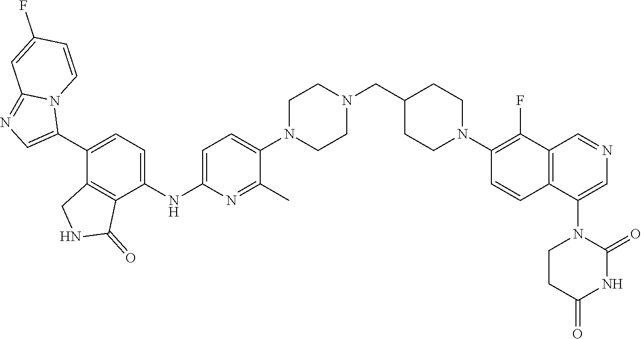 [SMILES: Cc1nc(Nc2ccc(-c3cnc4cc(F)ccn34)c3c2C(=O)NC3)ccc1N1CCN(CC2CCN(c3ccc4c(N5CCC(=O)NC5=O)cncc4c3F)CC2)CC1] |
| 287 | 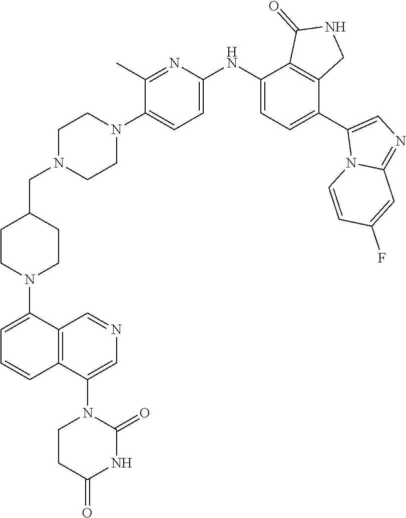 [SMILES: Cc1nc(Nc2ccc(-c3cnc4cc(F)ccn34)c3c2C(=O)NC3)ccc1N1CCN(CC2CCN(c3cccc4c(N5CCC(=O)NC5=O)cncc34)CC2)CC1] |
| 288 | 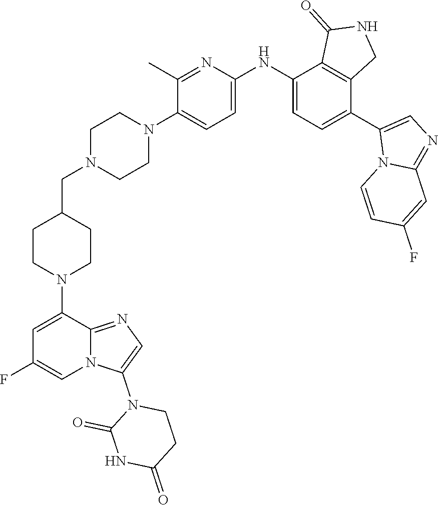 [SMILES: Cc1nc(Nc2ccc(-c3cnc4cc(F)ccn34)c3c2C(=O)NC3)ccc1N1CCN(CC2CCN(c3cc(F)cn4c(N5CCC(=O)NC5=O)cnc34)CC2)CC1] |
| 289 | 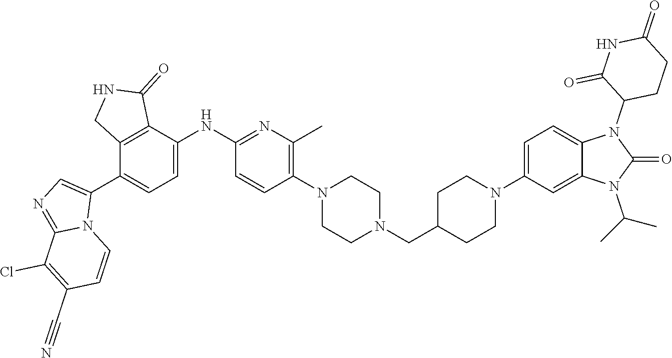 [SMILES: Cc1nc(Nc2ccc(-c3cnc4c(Cl)c(C#N)ccn34)c3c2C(=O)NC3)ccc1N1CCN(CC2CCN(c3ccc4c(c3)n(C(C)C)c(=O)n4C3CCC(=O)NC3=O)CC2)CC1] |
| 290 | 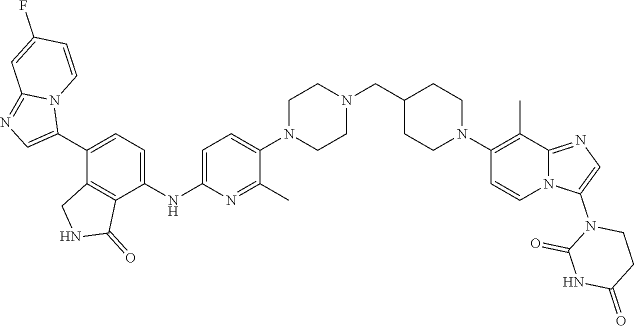 [SMILES: Cc1nc(Nc2ccc(-c3cnc4cc(F)ccn34)c3c2C(=O)NC3)ccc1N1CCN(CC2CCN(c3ccn4c(N5CCC(=O)NC5=O)cnc4c3C)CC2)CC1] |
| 291 | 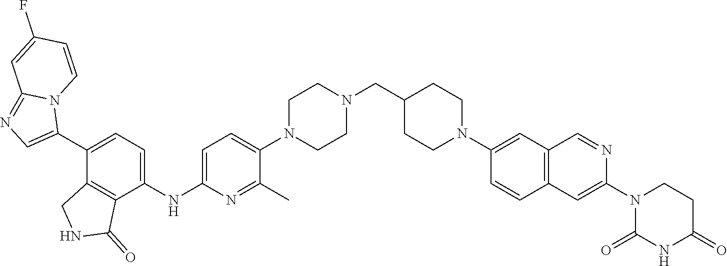 [SMILES: Cc1nc(Nc2ccc(-c3cnc4cc(F)ccn34)c3c2C(=O)NC3)ccc1N1CCN(CC2CCN(c3ccc4cc(N5CCC(=O)NC5=O)ncc4c3)CC2)CC1] |
| 292 | 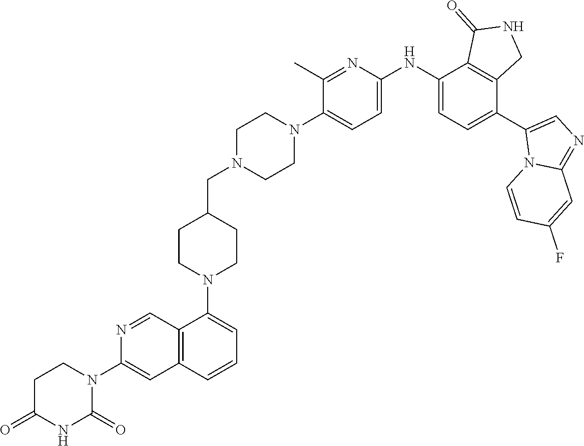 [SMILES: Cc1nc(Nc2ccc(-c3cnc4cc(F)ccn34)c3c2C(=O)NC3)ccc1N1CCN(CC2CCN(c3cccc4cc(N5CCC(=O)NC5=O)ncc34)CC2)CC1] |
| 293 | 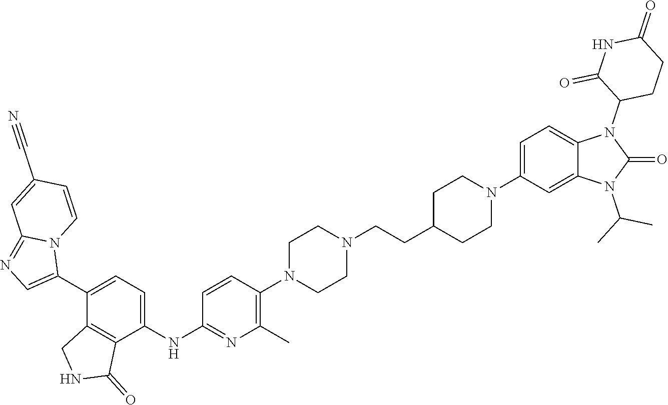 [SMILES: Cc1nc(Nc2ccc(-c3cnc4cc(C#N)ccn34)c3c2C(=O)NC3)ccc1N1CCN(CCC2CCN(c3ccc4c(c3)n(C(C)C)c(=O)n4C3CCC(=O)NC3=O)CC2)CC1] |
| 294 | 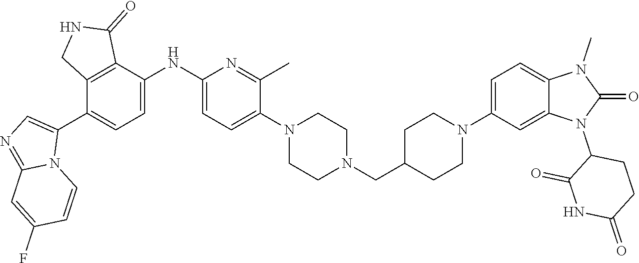 [SMILES: Cc1nc(Nc2ccc(-c3cnc4cc(F)ccn34)c3c2C(=O)NC3)ccc1N1CCN(CC2CCN(c3ccc4c(c3)n(C3CCC(=O)NC3=O)c(=O)n4C)CC2)CC1] |
| 295 | 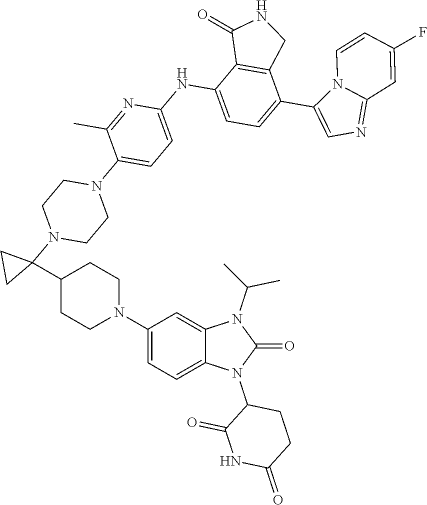 [SMILES: Cc1nc(Nc2ccc(-c3cnc4cc(F)ccn34)c3c2C(=O)NC3)ccc1N1CCN(C2(C3CCN(c4ccc5c(c4)n(C(C)C)c(=O)n5C4CCC(=O)NC4=O)CC3)CC2)CC1] |
| 296 | 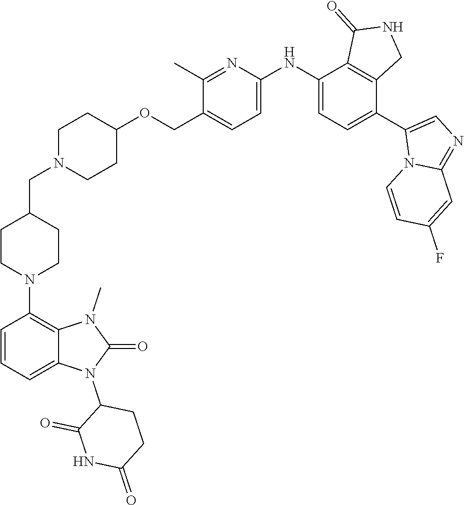 [SMILES: Cc1nc(Nc2ccc(-c3cnc4cc(F)ccn34)c3c2C(=O)NC3)ccc1COC1CCN(CC2CCN(c3cccc4c3n(C)c(=O)n4C3CCC(=O)NC3=O)CC2)CC1] |
| 297 | 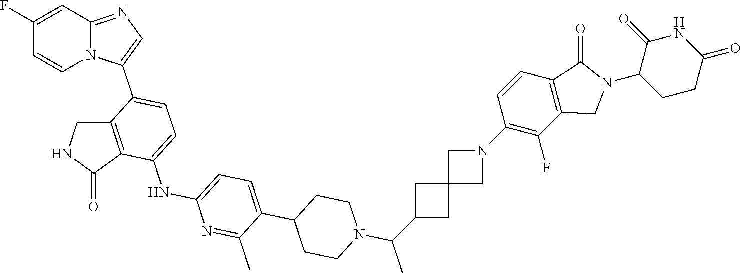 [SMILES: Cc1nc(Nc2ccc(-c3cnc4cc(F)ccn34)c3c2C(=O)NC3)ccc1C1CCN(C(C)C2CC3(C2)CN(c2ccc4c(c2F)CN(C2CCC(=O)NC2=O)C4=O)C3)CC1] |
| 298 | 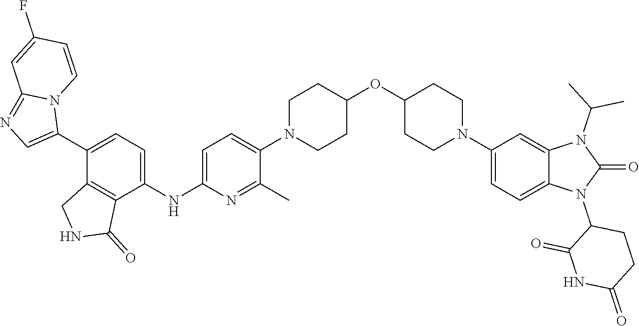 [SMILES: Cc1nc(Nc2ccc(-c3cnc4cc(F)ccn34)c3c2C(=O)NC3)ccc1N1CCC(OC2CCN(c3ccc4c(c3)n(C(C)C)c(=O)n4C3CCC(=O)NC3=O)CC2)CC1] |
| 299 | 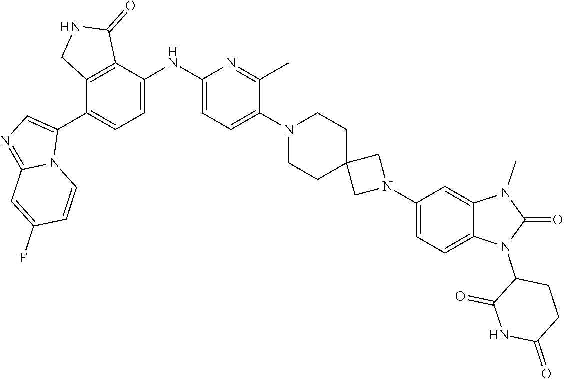 [SMILES: Cc1nc(Nc2ccc(-c3cnc4cc(F)ccn34)c3c2C(=O)NC3)ccc1N1CCC2(CC1)CN(c1ccc3c(c1)n(C)c(=O)n3C1CCC(=O)NC1=O)C2] |
| 300 | 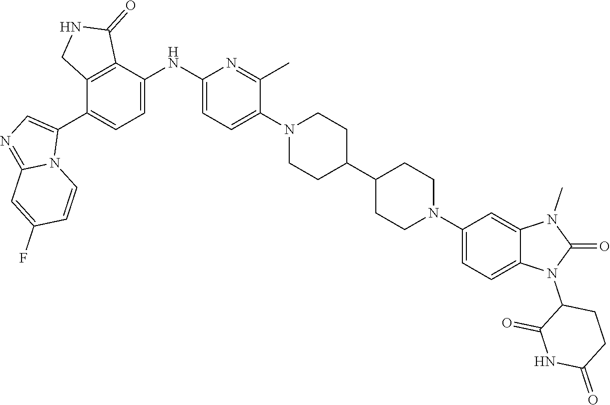 [SMILES: Cc1nc(Nc2ccc(-c3cnc4cc(F)ccn34)c3c2C(=O)NC3)ccc1N1CCC(C2CCN(c3ccc4c(c3)n(C)c(=O)n4C3CCC(=O)NC3=O)CC2)CC1] |
| 301 | 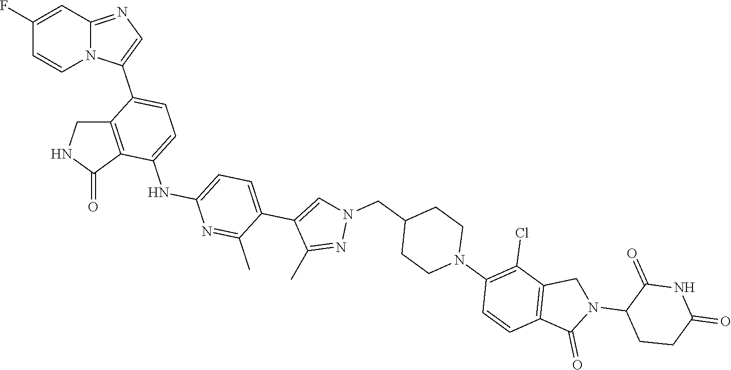 [SMILES: Cc1nc(Nc2ccc(-c3cnc4cc(F)ccn34)c3c2C(=O)NC3)ccc1-c1cn(CC2CCN(c3ccc4c(c3Cl)CN(C3CCC(=O)NC3=O)C4=O)CC2)nc1C] |
| 302 | 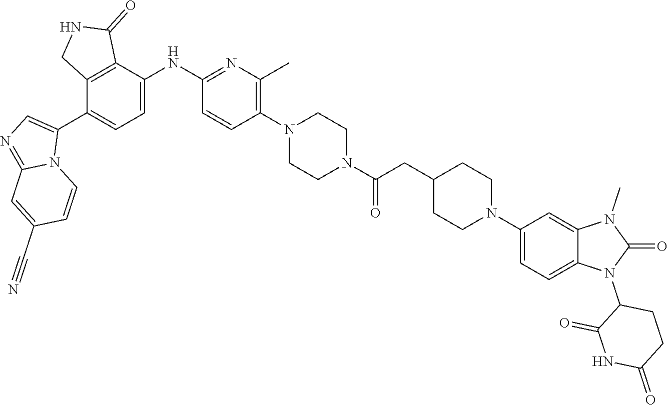 [SMILES: Cc1nc(Nc2ccc(-c3cnc4cc(C#N)ccn34)c3c2C(=O)NC3)ccc1N1CCN(C(=O)CC2CCN(c3ccc4c(c3)n(C)c(=O)n4C3CCC(=O)NC3=O)CC2)CC1] |
| 303 | 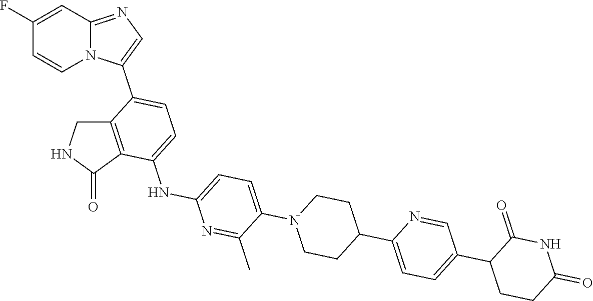 [SMILES: Cc1nc(Nc2ccc(-c3cnc4cc(F)ccn34)c3c2C(=O)NC3)ccc1N1CCC(c2ccc(C3CCC(=O)NC3=O)cn2)CC1] |
| 304 | 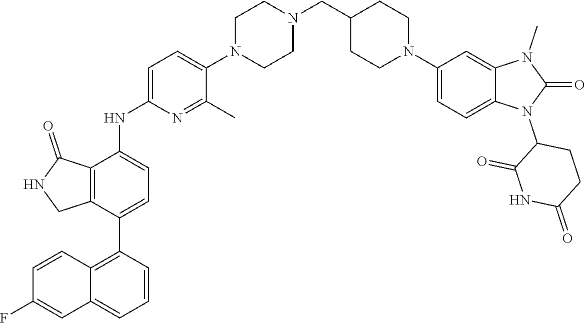 [SMILES: Cc1nc(Nc2ccc(-c3cccc4cc(F)ccc34)c3c2C(=O)NC3)ccc1N1CCN(CC2CCN(c3ccc4c(c3)n(C)c(=O)n4C3CCC(=O)NC3=O)CC2)CC1] |
| 305 | 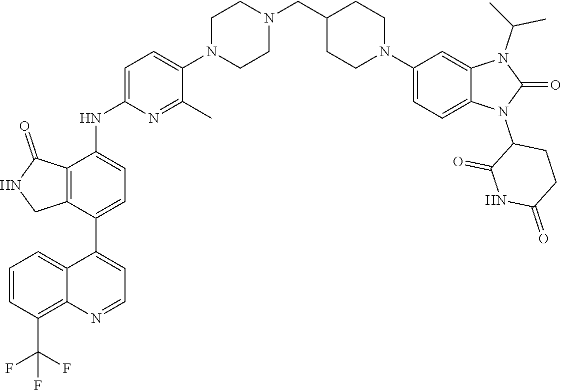 [SMILES: Cc1nc(Nc2ccc(-c3ccnc4c(C(F)(F)F)cccc34)c3c2C(=O)NC3)ccc1N1CCN(CC2CCN(c3ccc4c(c3)n(C(C)C)c(=O)n4C3CCC(=O)NC3=O)CC2)CC1] |
| 306 | 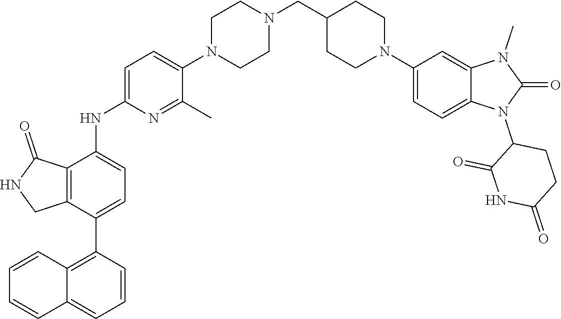 [SMILES: Cc1nc(Nc2ccc(-c3cccc4ccccc34)c3c2C(=O)NC3)ccc1N1CCN(CC2CCN(c3ccc4c(c3)n(C)c(=O)n4C3CCC(=O)NC3=O)CC2)CC1] |
| 307 | 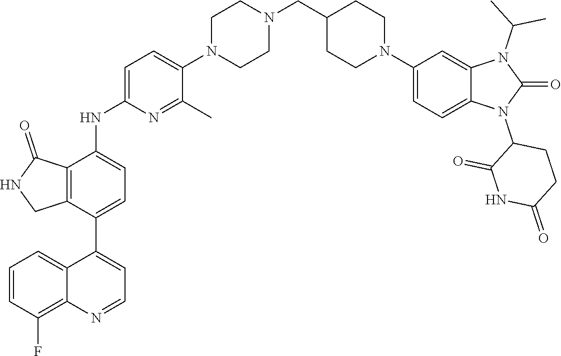 [SMILES: Cc1nc(Nc2ccc(-c3ccnc4c(F)cccc34)c3c2C(=O)NC3)ccc1N1CCN(CC2CCN(c3ccc4c(c3)n(C(C)C)c(=O)n4C3CCC(=O)NC3=O)CC2)CC1] |
| 308 | 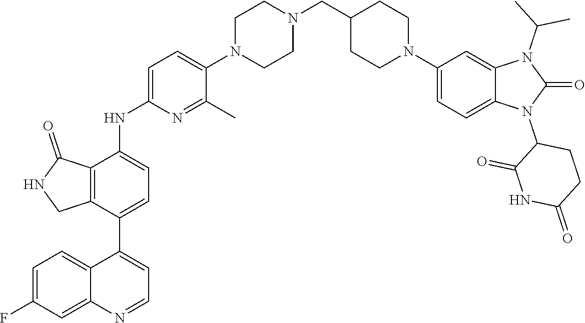 [SMILES: Cc1nc(Nc2ccc(-c3ccnc4cc(F)ccc34)c3c2C(=O)NC3)ccc1N1CCN(CC2CCN(c3ccc4c(c3)n(C(C)C)c(=O)n4C3CCC(=O)NC3=O)CC2)CC1] |
| 309 | 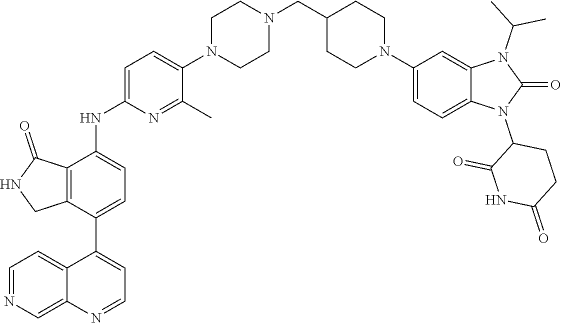 [SMILES: Cc1nc(Nc2ccc(-c3ccnc4cnccc34)c3c2C(=O)NC3)ccc1N1CCN(CC2CCN(c3ccc4c(c3)n(C(C)C)c(=O)n4C3CCC(=O)NC3=O)CC2)CC1] |
| 310 | 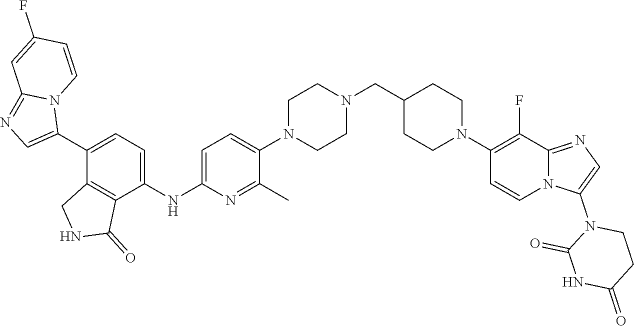 [SMILES: Cc1nc(Nc2ccc(-c3cnc4cc(F)ccn34)c3c2C(=O)NC3)ccc1N1CCN(CC2CCN(c3ccn4c(N5CCC(=O)NC5=O)cnc4c3F)CC2)CC1] |
| 311 | 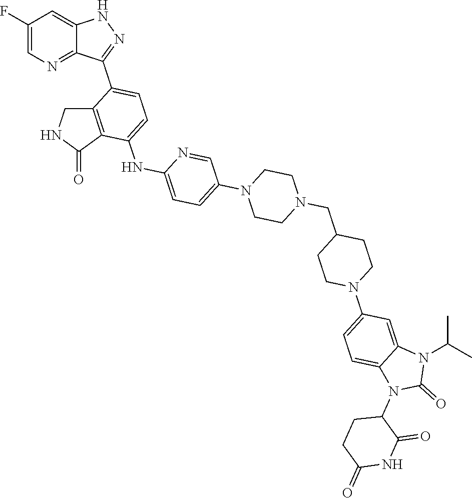 [SMILES: CC(C)n1c(=O)n(C2CCC(=O)NC2=O)c2ccc(N3CCC(CN4CCN(c5ccc(Nc6ccc(-c7n[nH]c8cc(F)cnc78)c7c6C(=O)NC7)nc5)CC4)CC3)cc21] |
| 312 | 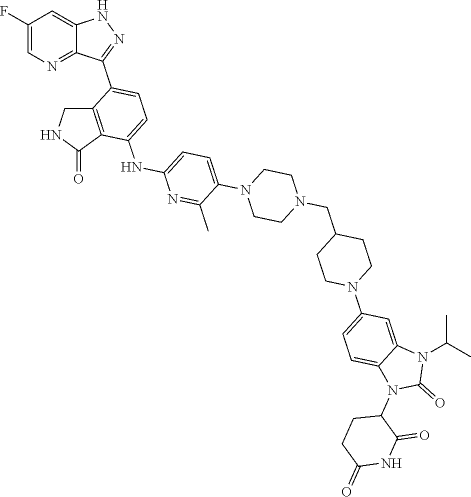 [SMILES: Cc1nc(Nc2ccc(-c3n[nH]c4cc(F)cnc34)c3c2C(=O)NC3)ccc1N1CCN(CC2CCN(c3ccc4c(c3)n(C(C)C)c(=O)n4C3CCC(=O)NC3=O)CC2)CC1] |
| 313 | 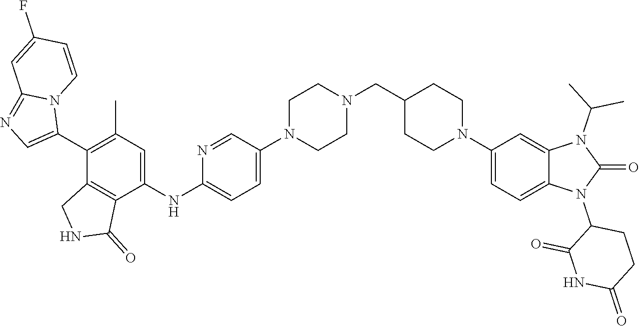 [SMILES: Cc1cc(Nc2ccc(N3CCN(CC4CCN(c5ccc6c(c5)n(C(C)C)c(=O)n6C5CCC(=O)NC5=O)CC4)CC3)cn2)c2c(c1-c1cnc3cc(F)ccn13)CNC2=O] |
| 314 | 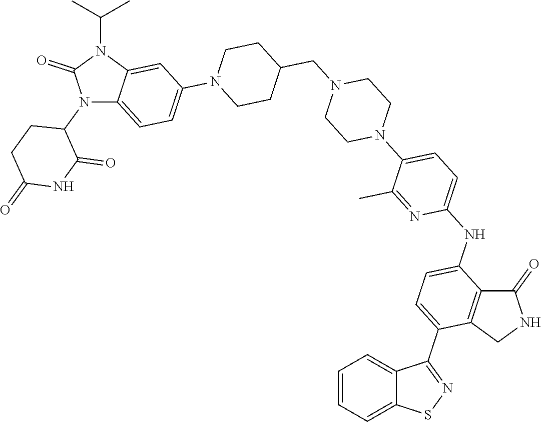 [SMILES: Cc1nc(Nc2ccc(-c3nsc4ccccc34)c3c2C(=O)NC3)ccc1N1CCN(CC2CCN(c3ccc4c(c3)n(C(C)C)c(=O)n4C3CCC(=O)NC3=O)CC2)CC1] |
| 315 | 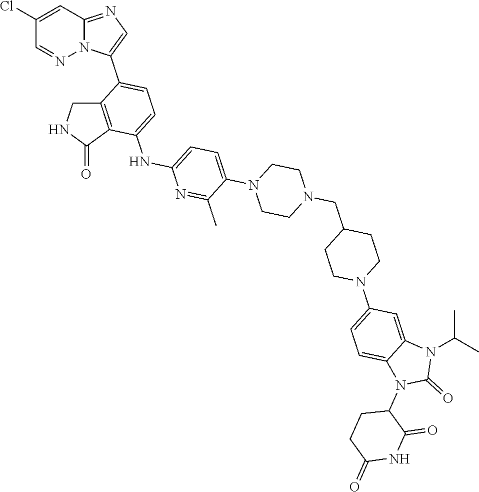 [SMILES: Cc1nc(Nc2ccc(-c3cnc4cc(Cl)cnn34)c3c2C(=O)NC3)ccc1N1CCN(CC2CCN(c3ccc4c(c3)n(C(C)C)c(=O)n4C3CCC(=O)NC3=O)CC2)CC1] |
| 316 | 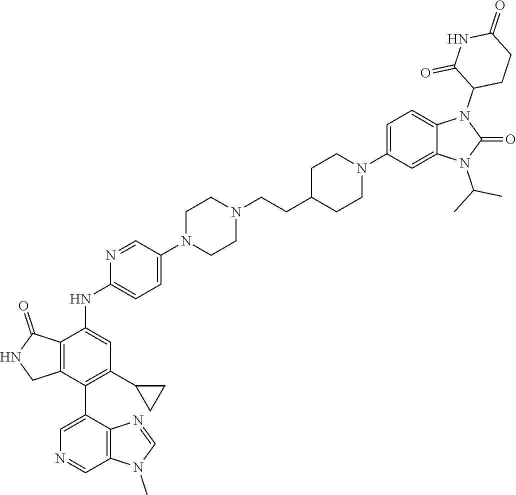 [SMILES: CC(C)n1c(=O)n(C2CCC(=O)NC2=O)c2ccc(N3CCC(CCN4CCN(c5ccc(Nc6cc(C7CC7)c(-c7cncc8c7ncn8C)c7c6C(=O)NC7)nc5)CC4)CC3)cc21] |
| 317 | 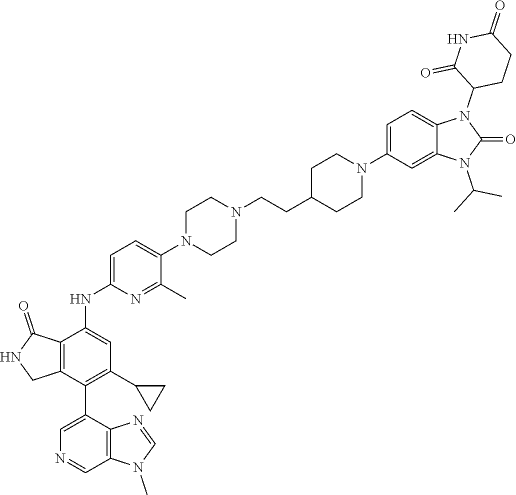 [SMILES: Cc1nc(Nc2cc(C3CC3)c(-c3cncc4c3ncn4C)c3c2C(=O)NC3)ccc1N1CCN(CCC2CCN(c3ccc4c(c3)n(C(C)C)c(=O)n4C3CCC(=O)NC3=O)CC2)CC1] |
| 318 | 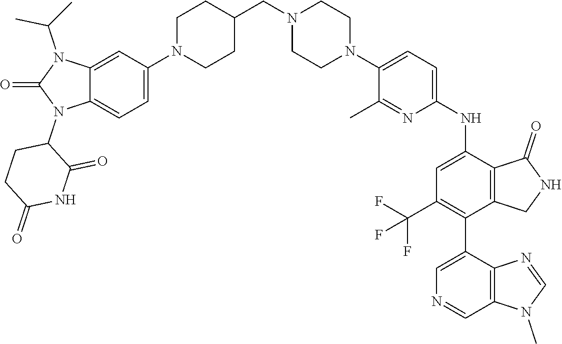 [SMILES: Cc1nc(Nc2cc(C(F)(F)F)c(-c3cncc4c3ncn4C)c3c2C(=O)NC3)ccc1N1CCN(CC2CCN(c3ccc4c(c3)n(C(C)C)c(=O)n4C3CCC(=O)NC3=O)CC2)CC1] |
| 319 | 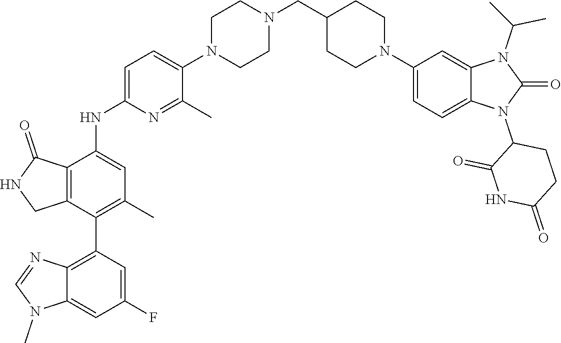 [SMILES: Cc1cc(Nc2ccc(N3CCN(CC4CCN(c5ccc6c(c5)n(C(C)C)c(=O)n6C5CCC(=O)NC5=O)CC4)CC3)c(C)n2)c2c(c1-c1cc(F)cc3c1ncn3C)CNC2=O] |
| 320 | 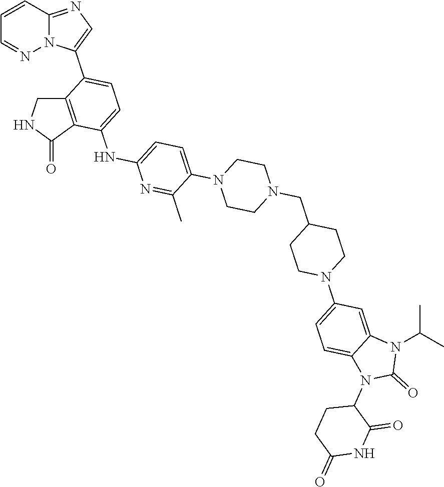 [SMILES: Cc1nc(Nc2ccc(-c3cnc4cccnn34)c3c2C(=O)NC3)ccc1N1CCN(CC2CCN(c3ccc4c(c3)n(C(C)C)c(=O)n4C3CCC(=O)NC3=O)CC2)CC1] |
| 321 | 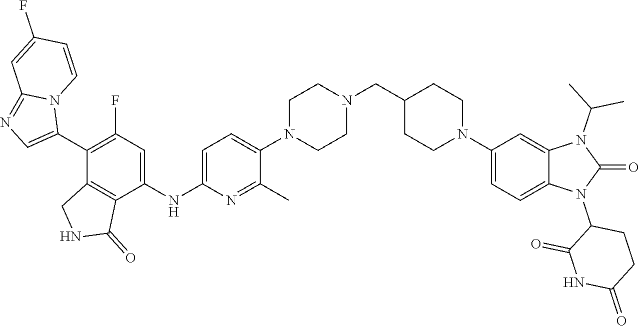 [SMILES: Cc1nc(Nc2cc(F)c(-c3cnc4cc(F)ccn34)c3c2C(=O)NC3)ccc1N1CCN(CC2CCN(c3ccc4c(c3)n(C(C)C)c(=O)n4C3CCC(=O)NC3=O)CC2)CC1] |
| 322 | 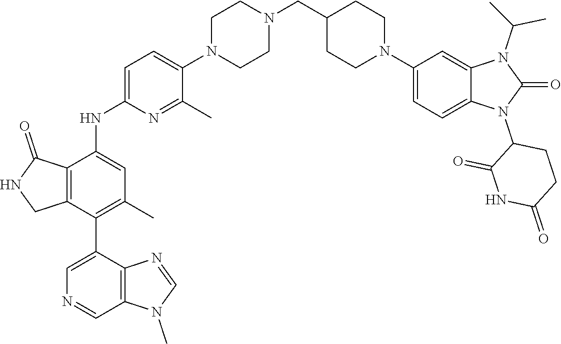 [SMILES: Cc1cc(Nc2ccc(N3CCN(CC4CCN(c5ccc6c(c5)n(C(C)C)c(=O)n6C5CCC(=O)NC5=O)CC4)CC3)c(C)n2)c2c(c1-c1cncc3c1ncn3C)CNC2=O] |
| 323 | 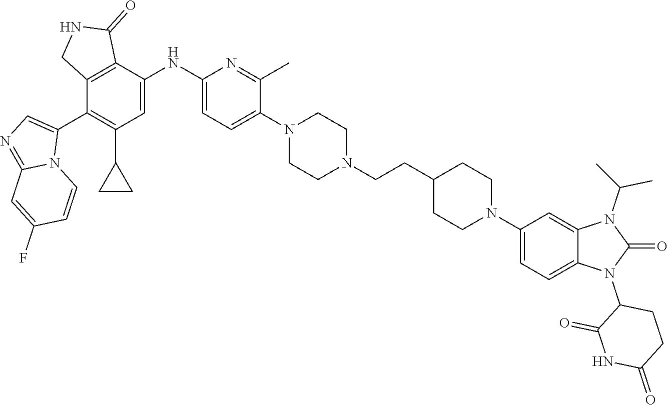 [SMILES: Cc1nc(Nc2cc(C3CC3)c(-c3cnc4cc(F)ccn34)c3c2C(=O)NC3)ccc1N1CCN(CCC2CCN(c3ccc4c(c3)n(C(C)C)c(=O)n4C3CCC(=O)NC3=O)CC2)CC1] |
| 324 | 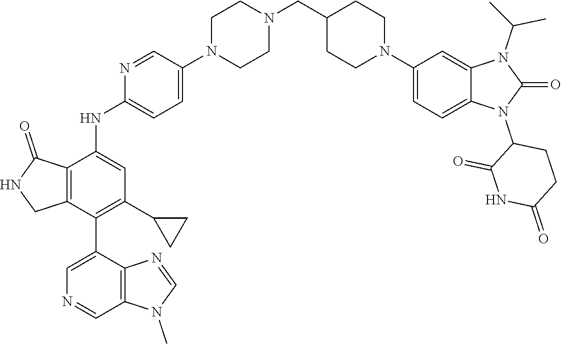 [SMILES: CC(C)n1c(=O)n(C2CCC(=O)NC2=O)c2ccc(N3CCC(CN4CCN(c5ccc(Nc6cc(C7CC7)c(-c7cncc8c7ncn8C)c7c6C(=O)NC7)nc5)CC4)CC3)cc21] |
| 325 | 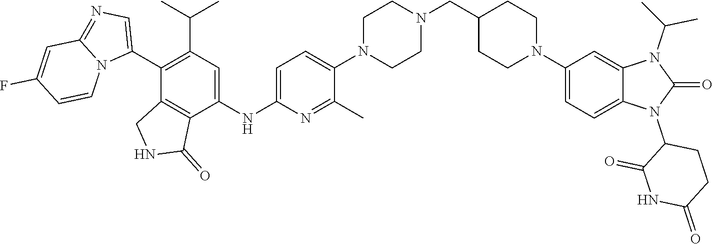 [SMILES: Cc1nc(Nc2cc(C(C)C)c(-c3cnc4cc(F)ccn34)c3c2C(=O)NC3)ccc1N1CCN(CC2CCN(c3ccc4c(c3)n(C(C)C)c(=O)n4C3CCC(=O)NC3=O)CC2)CC1] |
| 326 | 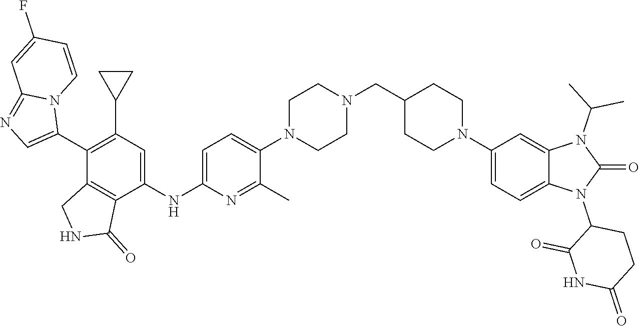 [SMILES: Cc1nc(Nc2cc(C3CC3)c(-c3cnc4cc(F)ccn34)c3c2C(=O)NC3)ccc1N1CCN(CC2CCN(c3ccc4c(c3)n(C(C)C)c(=O)n4C3CCC(=O)NC3=O)CC2)CC1] |
| 327 | 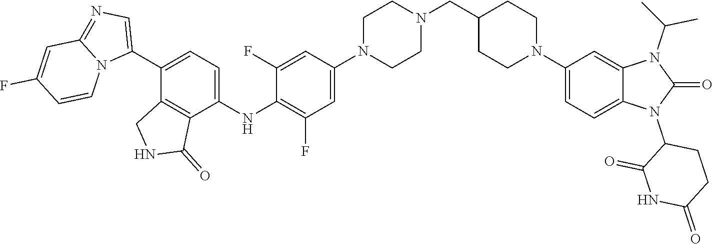 [SMILES: CC(C)n1c(=O)n(C2CCC(=O)NC2=O)c2ccc(N3CCC(CN4CCN(c5cc(F)c(Nc6ccc(-c7cnc8cc(F)ccn78)c7c6C(=O)NC7)c(F)c5)CC4)CC3)cc21] |
| 328 | 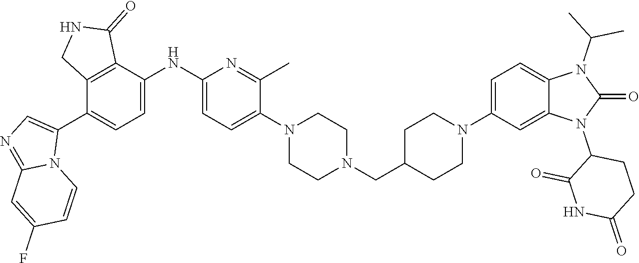 [SMILES: Cc1nc(Nc2ccc(-c3cnc4cc(F)ccn34)c3c2C(=O)NC3)ccc1N1CCN(CC2CCN(c3ccc4c(c3)n(C3CCC(=O)NC3=O)c(=O)n4C(C)C)CC2)CC1] |
| 329 | 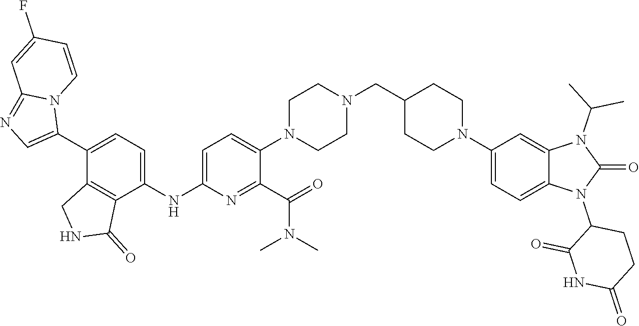 [SMILES: CC(C)n1c(=O)n(C2CCC(=O)NC2=O)c2ccc(N3CCC(CN4CCN(c5ccc(Nc6ccc(-c7cnc8cc(F)ccn78)c7c6C(=O)NC7)nc5C(=O)N(C)C)CC4)CC3)cc21] |
| 330 | 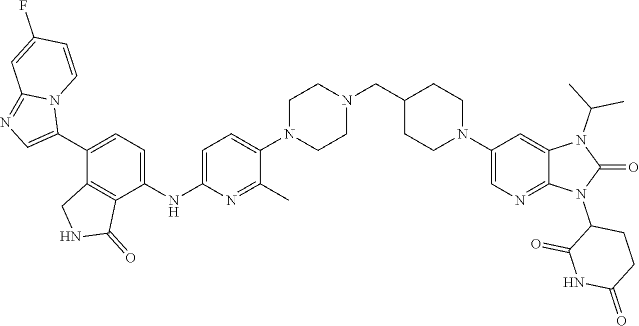 [SMILES: Cc1nc(Nc2ccc(-c3cnc4cc(F)ccn34)c3c2C(=O)NC3)ccc1N1CCN(CC2CCN(c3cnc4c(c3)n(C(C)C)c(=O)n4C3CCC(=O)NC3=O)CC2)CC1] |
| 331 | 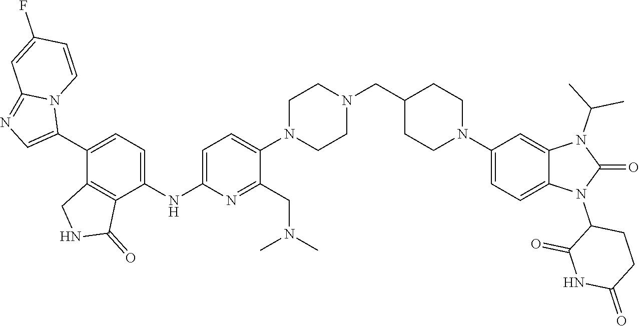 [SMILES: CC(C)n1c(=O)n(C2CCC(=O)NC2=O)c2ccc(N3CCC(CN4CCN(c5ccc(Nc6ccc(-c7cnc8cc(F)ccn78)c7c6C(=O)NC7)nc5CN(C)C)CC4)CC3)cc21] |
| 332 | 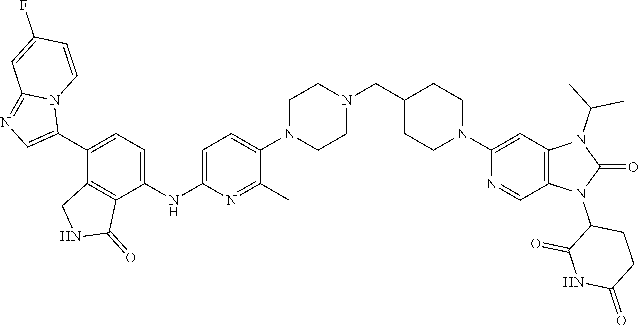 [SMILES: Cc1nc(Nc2ccc(-c3cnc4cc(F)ccn34)c3c2C(=O)NC3)ccc1N1CCN(CC2CCN(c3cc4c(cn3)n(C3CCC(=O)NC3=O)c(=O)n4C(C)C)CC2)CC1] |
| 333 | 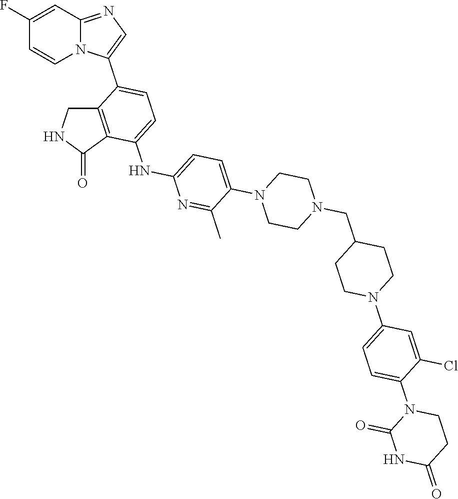 [SMILES: Cc1nc(Nc2ccc(-c3cnc4cc(F)ccn34)c3c2C(=O)NC3)ccc1N1CCN(CC2CCN(c3ccc(N4CCC(=O)NC4=O)c(Cl)c3)CC2)CC1] |
| 334 | 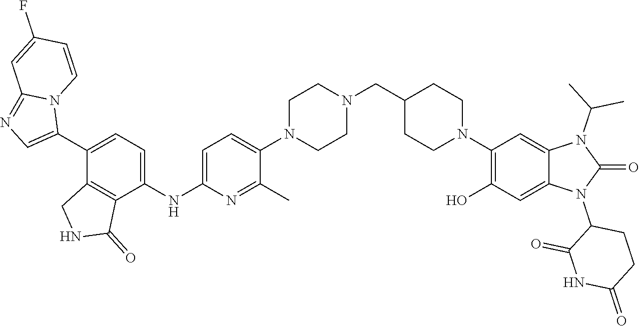 [SMILES: Cc1nc(Nc2ccc(-c3cnc4cc(F)ccn34)c3c2C(=O)NC3)ccc1N1CCN(CC2CCN(c3cc4c(cc3O)n(C3CCC(=O)NC3=O)c(=O)n4C(C)C)CC2)CC1] |
| 335 | 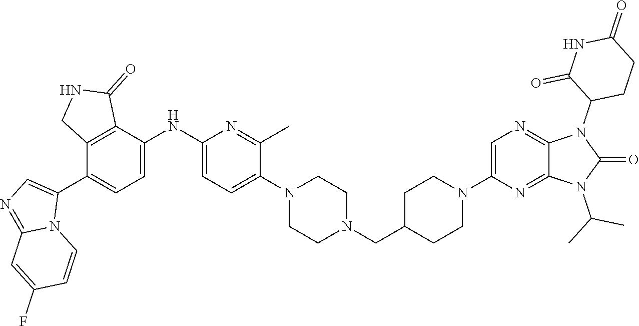 [SMILES: Cc1nc(Nc2ccc(-c3cnc4cc(F)ccn34)c3c2C(=O)NC3)ccc1N1CCN(CC2CCN(c3cnc4c(n3)n(C(C)C)c(=O)n4C3CCC(=O)NC3=O)CC2)CC1] |
| 336 | 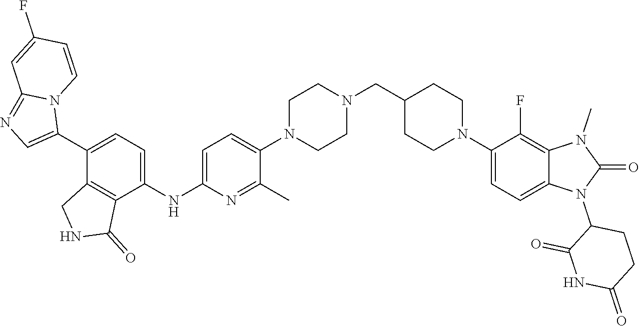 [SMILES: Cc1nc(Nc2ccc(-c3cnc4cc(F)ccn34)c3c2C(=O)NC3)ccc1N1CCN(CC2CCN(c3ccc4c(c3F)n(C)c(=O)n4C3CCC(=O)NC3=O)CC2)CC1] |
| 337 | 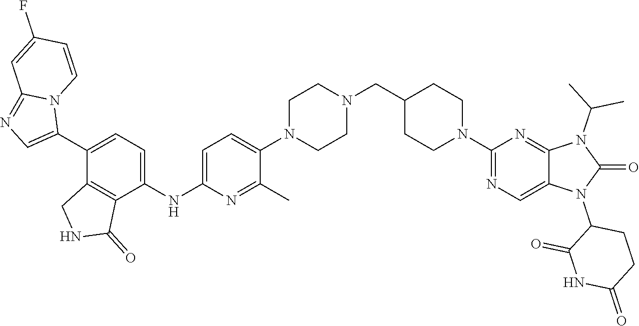 [SMILES: Cc1nc(Nc2ccc(-c3cnc4cc(F)ccn34)c3c2C(=O)NC3)ccc1N1CCN(CC2CCN(c3ncc4c(n3)n(C(C)C)c(=O)n4C3CCC(=O)NC3=O)CC2)CC1] |
| 338 | 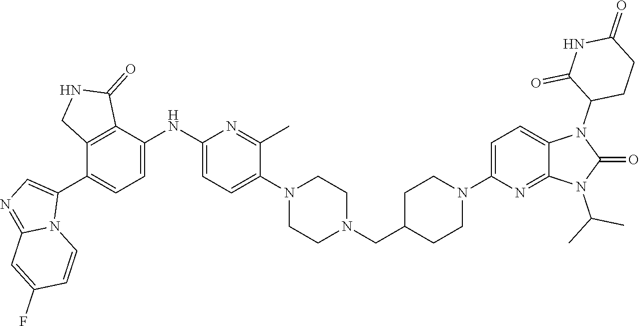 [SMILES: Cc1nc(Nc2ccc(-c3cnc4cc(F)ccn34)c3c2C(=O)NC3)ccc1N1CCN(CC2CCN(c3ccc4c(n3)n(C(C)C)c(=O)n4C3CCC(=O)NC3=O)CC2)CC1] |
| 339 | 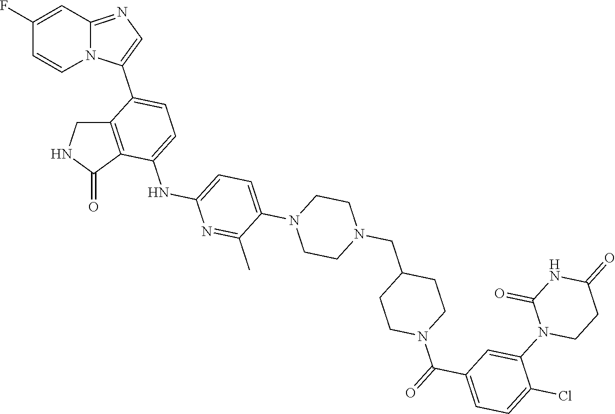 [SMILES: Cc1nc(Nc2ccc(-c3cnc4cc(F)ccn34)c3c2C(=O)NC3)ccc1N1CCN(CC2CCN(C(=O)c3ccc(Cl)c(N4CCC(=O)NC4=O)c3)CC2)CC1] |
| 340 | 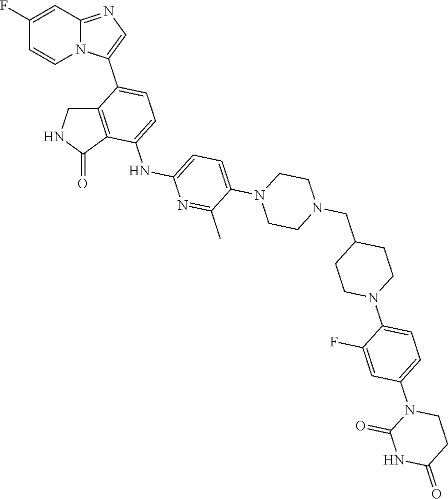 [SMILES: Cc1nc(Nc2ccc(-c3cnc4cc(F)ccn34)c3c2C(=O)NC3)ccc1N1CCN(CC2CCN(c3ccc(N4CCC(=O)NC4=O)cc3F)CC2)CC1] |
| 341 | 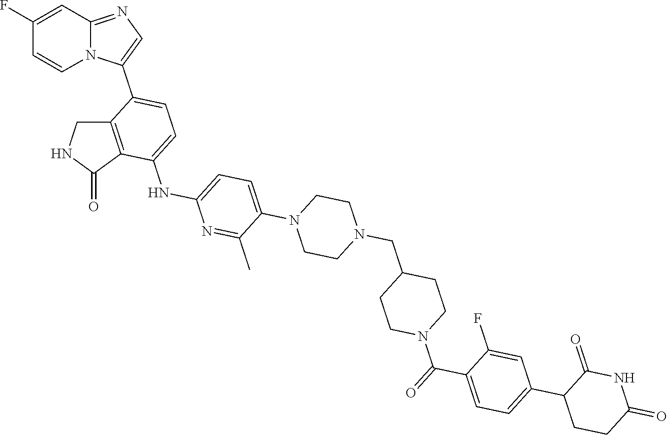 [SMILES: Cc1nc(Nc2ccc(-c3cnc4cc(F)ccn34)c3c2C(=O)NC3)ccc1N1CCN(CC2CCN(C(=O)c3ccc(C4CCC(=O)NC4=O)cc3F)CC2)CC1] |
| 342 | 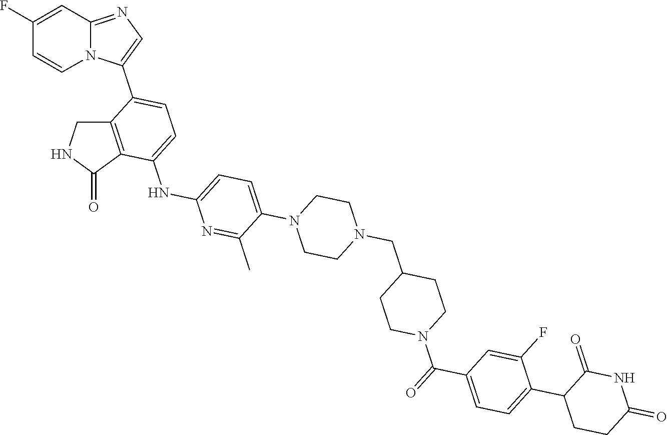 [SMILES: Cc1nc(Nc2ccc(-c3cnc4cc(F)ccn34)c3c2C(=O)NC3)ccc1N1CCN(CC2CCN(C(=O)c3ccc(C4CCC(=O)NC4=O)c(F)c3)CC2)CC1] |
| 343 | 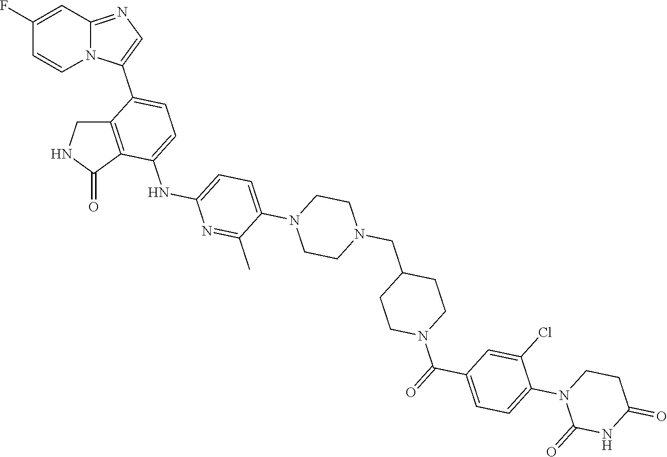 [SMILES: Cc1nc(Nc2ccc(-c3cnc4cc(F)ccn34)c3c2C(=O)NC3)ccc1N1CCN(CC2CCN(C(=O)c3ccc(N4CCC(=O)NC4=O)c(Cl)c3)CC2)CC1] |
| 344 | 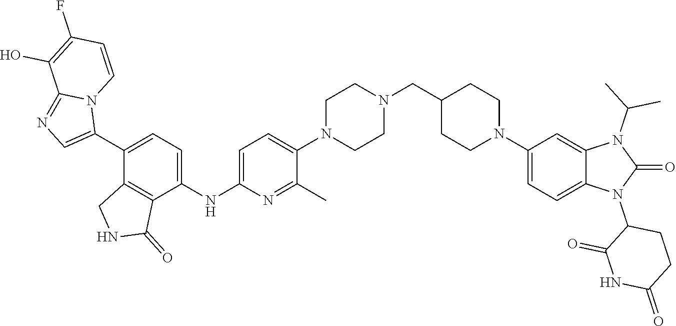 [SMILES: Cc1nc(Nc2ccc(-c3cnc4c(O)c(F)ccn34)c3c2C(=O)NC3)ccc1N1CCN(CC2CCN(c3ccc4c(c3)n(C(C)C)c(=O)n4C3CCC(=O)NC3=O)CC2)CC1] |
| 345 | 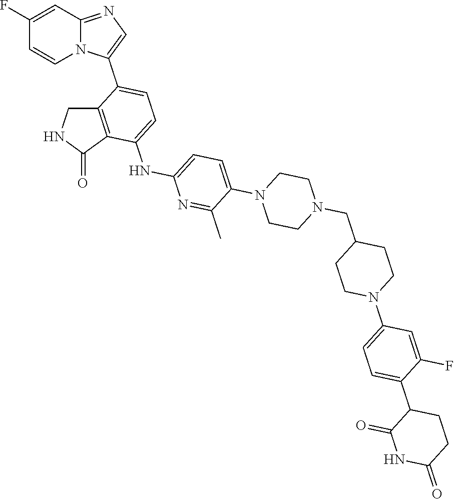 [SMILES: Cc1nc(Nc2ccc(-c3cnc4cc(F)ccn34)c3c2C(=O)NC3)ccc1N1CCN(CC2CCN(c3ccc(C4CCC(=O)NC4=O)c(F)c3)CC2)CC1] |
| 346 | 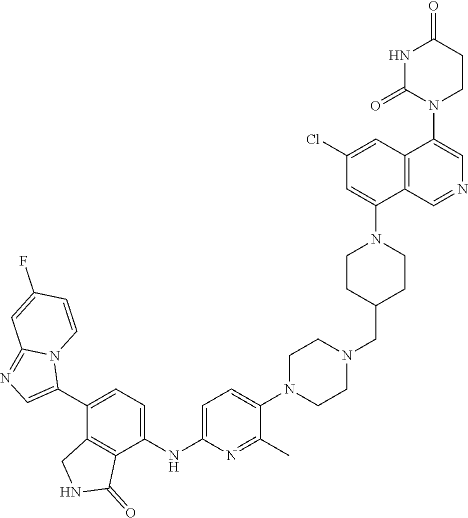 [SMILES: Cc1nc(Nc2ccc(-c3cnc4cc(F)ccn34)c3c2C(=O)NC3)ccc1N1CCN(CC2CCN(c3cc(Cl)cc4c(N5CCC(=O)NC5=O)cncc34)CC2)CC1] |
| 347 | 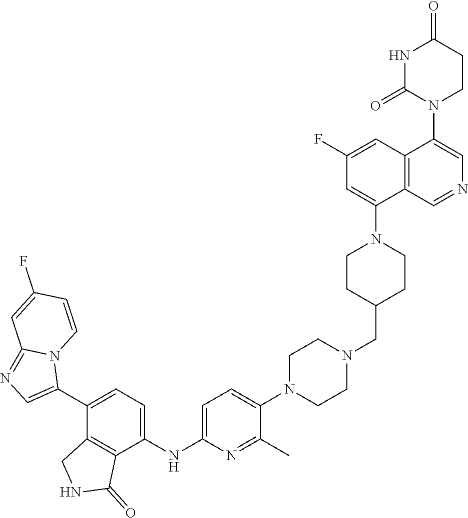 [SMILES: Cc1nc(Nc2ccc(-c3cnc4cc(F)ccn34)c3c2C(=O)NC3)ccc1N1CCN(CC2CCN(c3cc(F)cc4c(N5CCC(=O)NC5=O)cncc34)CC2)CC1] |
| 348 | 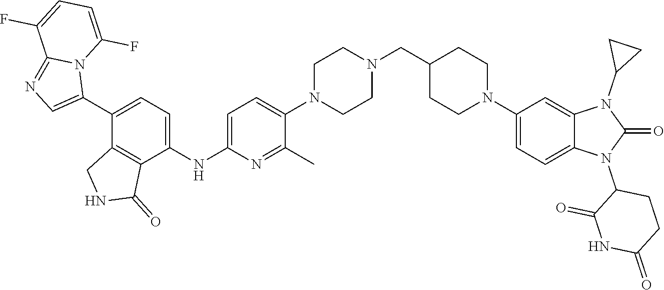 [SMILES: Cc1nc(Nc2ccc(-c3cnc4c(F)ccc(F)n34)c3c2C(=O)NC3)ccc1N1CCN(CC2CCN(c3ccc4c(c3)n(C3CC3)c(=O)n4C3CCC(=O)NC3=O)CC2)CC1] |
| 349 | 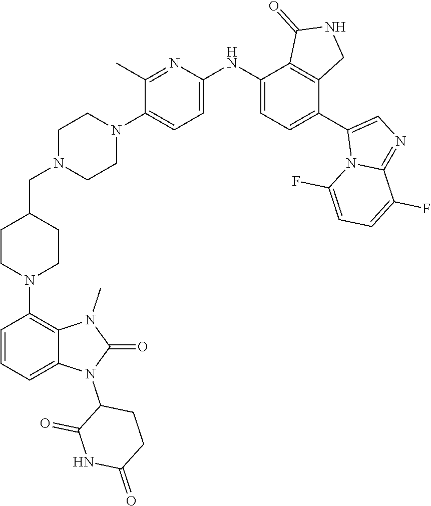 [SMILES: Cc1nc(Nc2ccc(-c3cnc4c(F)ccc(F)n34)c3c2C(=O)NC3)ccc1N1CCN(CC2CCN(c3cccc4c3n(C)c(=O)n4C3CCC(=O)NC3=O)CC2)CC1] |
| 350 | 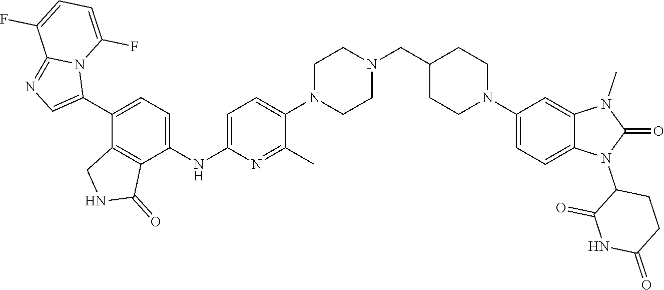 [SMILES: Cc1nc(Nc2ccc(-c3cnc4c(F)ccc(F)n34)c3c2C(=O)NC3)ccc1N1CCN(CC2CCN(c3ccc4c(c3)n(C)c(=O)n4C3CCC(=O)NC3=O)CC2)CC1] |
| 351 | 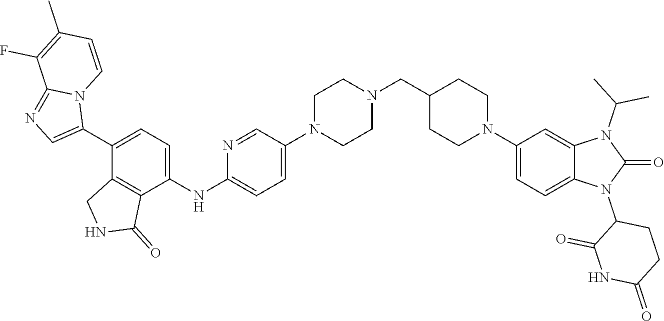 [SMILES: Cc1ccn2c(-c3ccc(Nc4ccc(N5CCN(CC6CCN(c7ccc8c(c7)n(C(C)C)c(=O)n8C7CCC(=O)NC7=O)CC6)CC5)cn4)c4c3CNC4=O)cnc2c1F] |
| 352 | 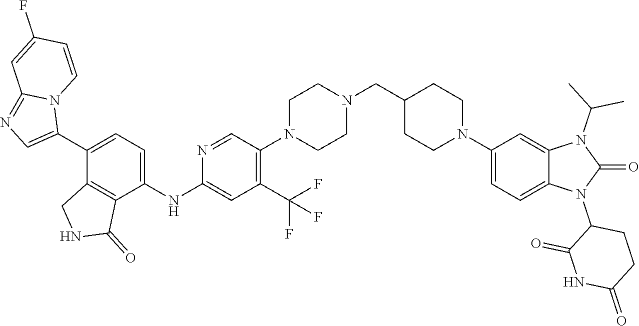 [SMILES: CC(C)n1c(=O)n(C2CCC(=O)NC2=O)c2ccc(N3CCC(CN4CCN(c5cnc(Nc6ccc(-c7cnc8cc(F)ccn78)c7c6C(=O)NC7)cc5C(F)(F)F)CC4)CC3)cc21] |
| 353 | 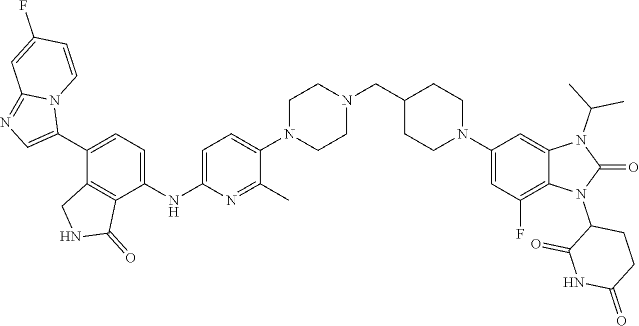 [SMILES: Cc1nc(Nc2ccc(-c3cnc4cc(F)ccn34)c3c2C(=O)NC3)ccc1N1CCN(CC2CCN(c3cc(F)c4c(c3)n(C(C)C)c(=O)n4C3CCC(=O)NC3=O)CC2)CC1] |
| 354 | 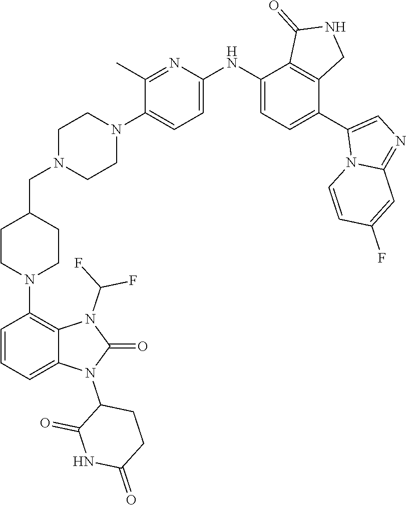 [SMILES: Cc1nc(Nc2ccc(-c3cnc4cc(F)ccn34)c3c2C(=O)NC3)ccc1N1CCN(CC2CCN(c3cccc4c3n(C(F)F)c(=O)n4C3CCC(=O)NC3=O)CC2)CC1] |
| 355 | 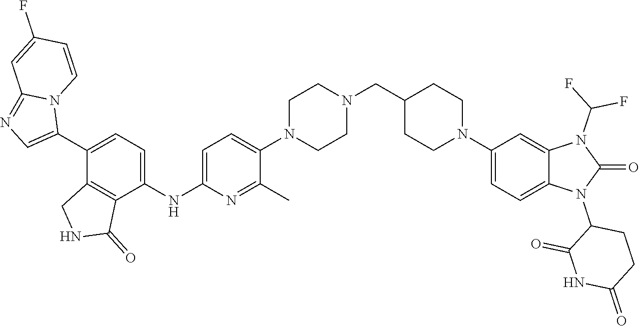 [SMILES: Cc1nc(Nc2ccc(-c3cnc4cc(F)ccn34)c3c2C(=O)NC3)ccc1N1CCN(CC2CCN(c3ccc4c(c3)n(C(F)F)c(=O)n4C3CCC(=O)NC3=O)CC2)CC1] |
| 356 | 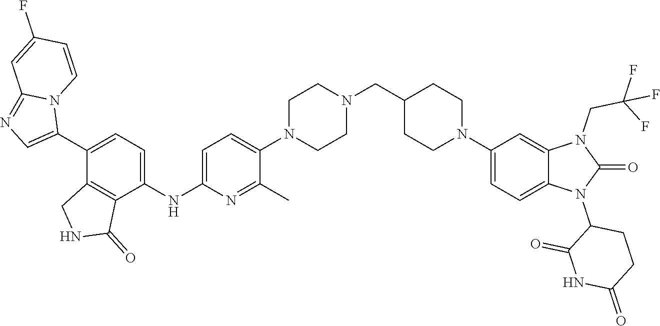 [SMILES: Cc1nc(Nc2ccc(-c3cnc4cc(F)ccn34)c3c2C(=O)NC3)ccc1N1CCN(CC2CCN(c3ccc4c(c3)n(CC(F)(F)F)c(=O)n4C3CCC(=O)NC3=O)CC2)CC1] |
| 357 | 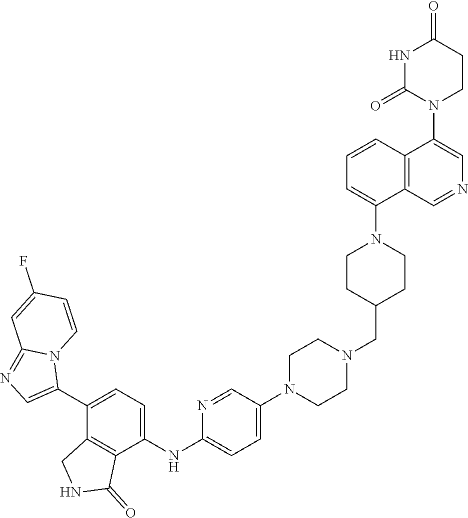 [SMILES: O=C1CCN(c2cncc3c(N4CCC(CN5CCN(c6ccc(Nc7ccc(-c8cnc9cc(F)ccn89)c8c7C(=O)NC8)nc6)CC5)CC4)cccc23)C(=O)N1] |
| 358 | 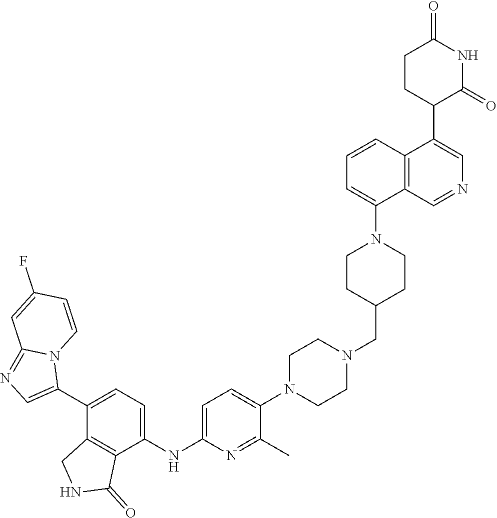 [SMILES: Cc1nc(Nc2ccc(-c3cnc4cc(F)ccn34)c3c2C(=O)NC3)ccc1N1CCN(CC2CCN(c3cccc4c(C5CCC(=O)NC5=O)cncc34)CC2)CC1] |
| 359 | 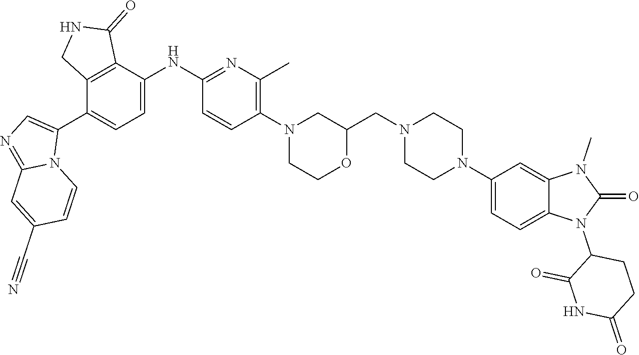 [SMILES: Cc1nc(Nc2ccc(-c3cnc4cc(C#N)ccn34)c3c2C(=O)NC3)ccc1N1CCOC(CN2CCN(c3ccc4c(c3)n(C)c(=O)n4C3CCC(=O)NC3=O)CC2)C1] |
| 360 | 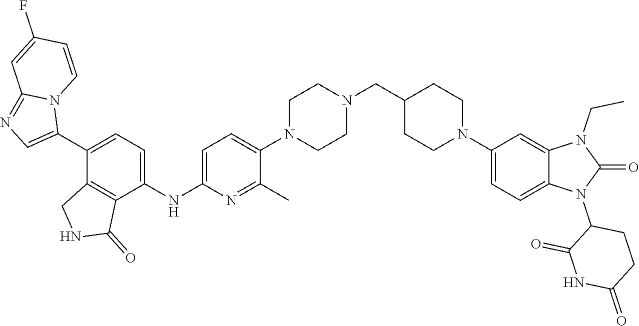 [SMILES: CCn1c(=O)n(C2CCC(=O)NC2=O)c2ccc(N3CCC(CN4CCN(c5ccc(Nc6ccc(-c7cnc8cc(F)ccn78)c7c6C(=O)NC7)nc5C)CC4)CC3)cc21] |
| 361 | 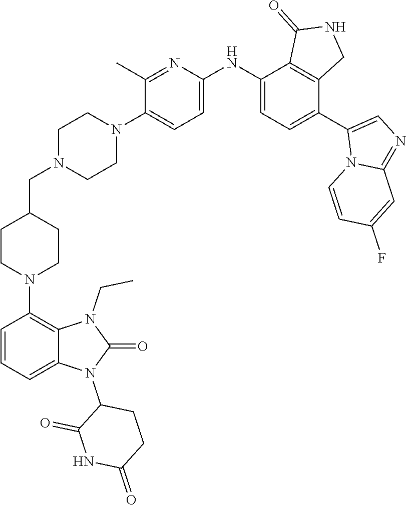 [SMILES: CCn1c(=O)n(C2CCC(=O)NC2=O)c2cccc(N3CCC(CN4CCN(c5ccc(Nc6ccc(-c7cnc8cc(F)ccn78)c7c6C(=O)NC7)nc5C)CC4)CC3)c21] |
| 362 | 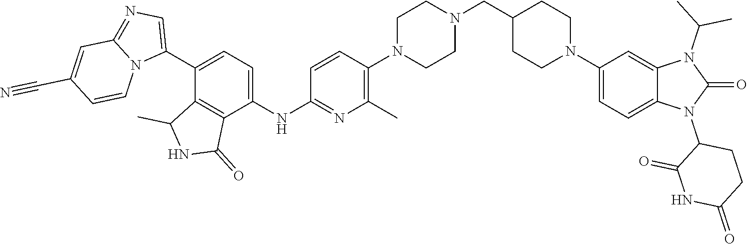 [SMILES: Cc1nc(Nc2ccc(-c3cnc4cc(C#N)ccn34)c3c2C(=O)NC3C)ccc1N1CCN(CC2CCN(c3ccc4c(c3)n(C(C)C)c(=O)n4C3CCC(=O)NC3=O)CC2)CC1] |
| 363 | 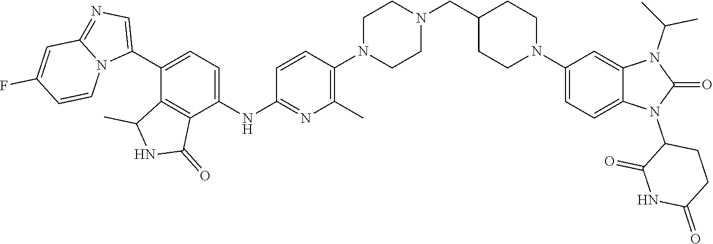 [SMILES: Cc1nc(Nc2ccc(-c3cnc4cc(F)ccn34)c3c2C(=O)NC3C)ccc1N1CCN(CC2CCN(c3ccc4c(c3)n(C(C)C)c(=O)n4C3CCC(=O)NC3=O)CC2)CC1] |
| 364 | 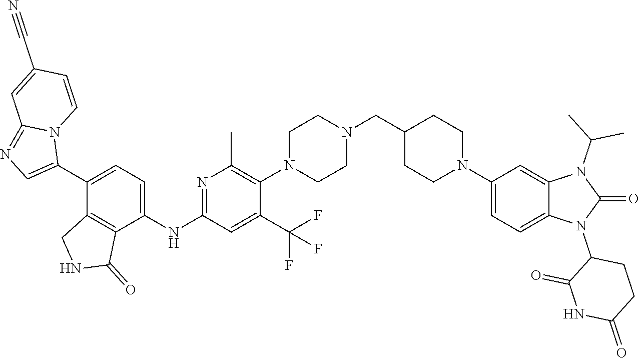 [SMILES: Cc1nc(Nc2ccc(-c3cnc4cc(C#N)ccn34)c3c2C(=O)NC3)cc(C(F)(F)F)c1N1CCN(CC2CCN(c3ccc4c(c3)n(C(C)C)c(=O)n4C3CCC(=O)NC3=O)CC2)CC1] |
| 365 | 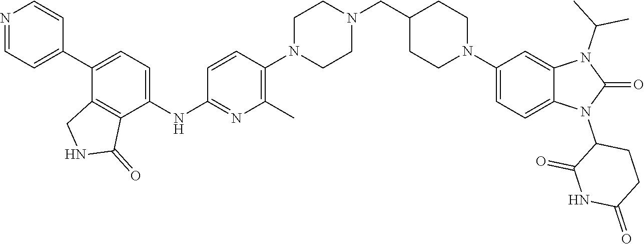 [SMILES: Cc1nc(Nc2ccc(-c3ccncc3)c3c2C(=O)NC3)ccc1N1CCN(CC2CCN(c3ccc4c(c3)n(C(C)C)c(=O)n4C3CCC(=O)NC3=O)CC2)CC1] |
| 366 | 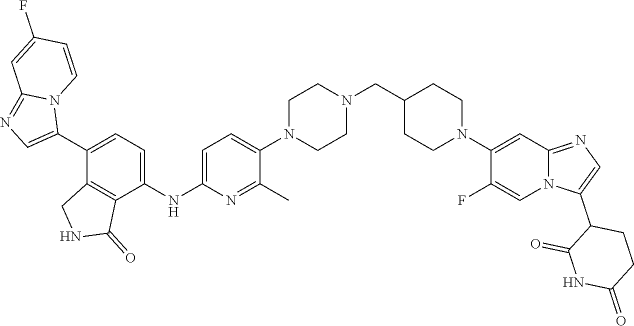 [SMILES: Cc1nc(Nc2ccc(-c3cnc4cc(F)ccn34)c3c2C(=O)NC3)ccc1N1CCN(CC2CCN(c3cc4ncc(C5CCC(=O)NC5=O)n4cc3F)CC2)CC1] |
| 367 | 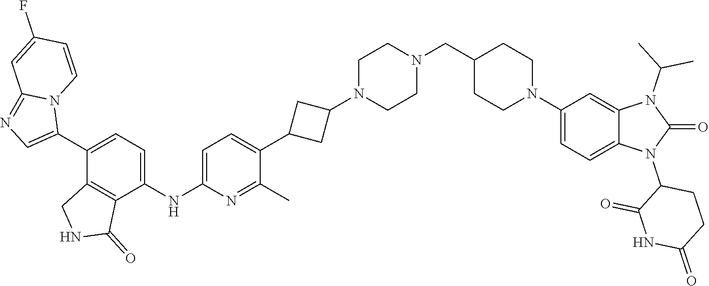 [SMILES: Cc1nc(Nc2ccc(-c3cnc4cc(F)ccn34)c3c2C(=O)NC3)ccc1C1CC(N2CCN(CC3CCN(c4ccc5c(c4)n(C(C)C)c(=O)n5C4CCC(=O)NC4=O)CC3)CC2)C1] |
| 368 | 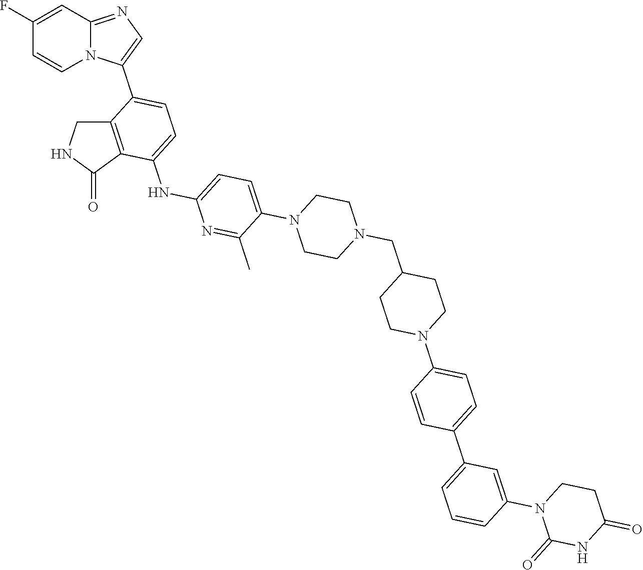 [SMILES: Cc1nc(Nc2ccc(-c3cnc4cc(F)ccn34)c3c2C(=O)NC3)ccc1N1CCN(CC2CCN(c3ccc(-c4cccc(N5CCC(=O)NC5=O)c4)cc3)CC2)CC1] |
| 369 | 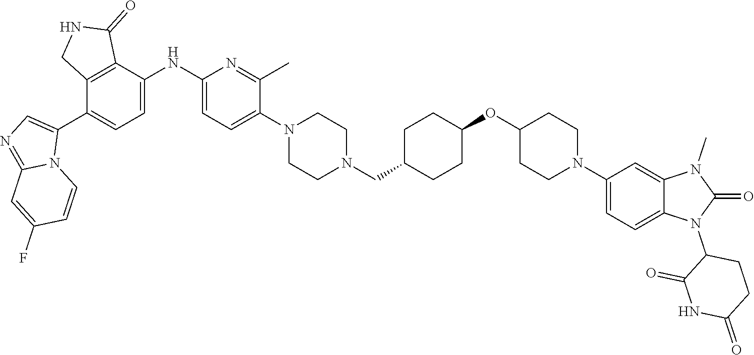 [SMILES: Cc1nc(Nc2ccc(-c3cnc4cc(F)ccn34)c3c2C(=O)NC3)ccc1N1CCN(C[C@H]2CC[C@H](OC3CCN(c4ccc5c(c4)n(C)c(=O)n5C4CCC(=O)NC4=O)CC3)CC2)CC1] |
| 370 | 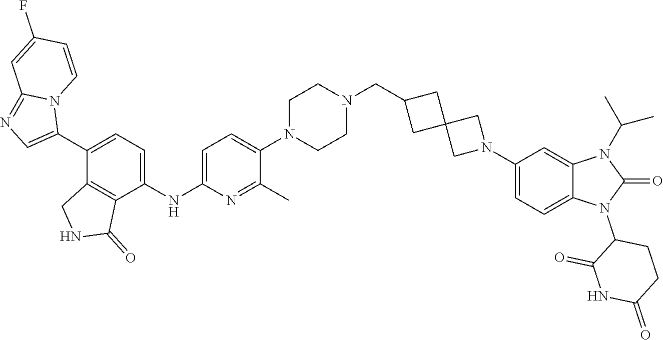 [SMILES: Cc1nc(Nc2ccc(-c3cnc4cc(F)ccn34)c3c2C(=O)NC3)ccc1N1CCN(CC2CC3(C2)CN(c2ccc4c(c2)n(C(C)C)c(=O)n4C2CCC(=O)NC2=O)C3)CC1] |
| 371 | 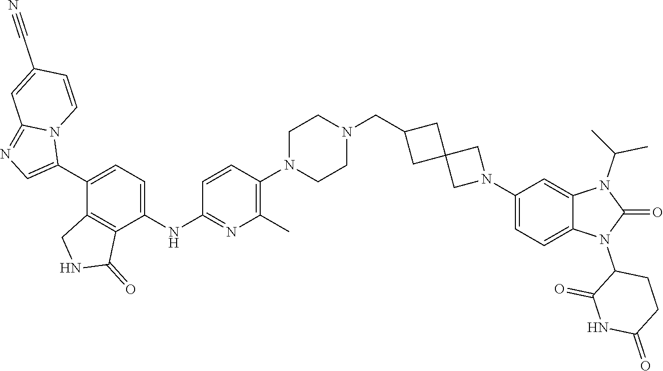 [SMILES: Cc1nc(Nc2ccc(-c3cnc4cc(C#N)ccn34)c3c2C(=O)NC3)ccc1N1CCN(CC2CC3(C2)CN(c2ccc4c(c2)n(C(C)C)c(=O)n4C2CCC(=O)NC2=O)C3)CC1] |
| 372 | 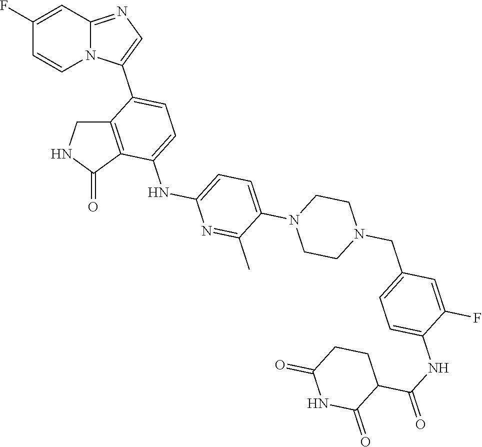 [SMILES: Cc1nc(Nc2ccc(-c3cnc4cc(F)ccn34)c3c2C(=O)NC3)ccc1N1CCN(Cc2ccc(NC(=O)C3CCC(=O)NC3=O)c(F)c2)CC1] |
| 373 | 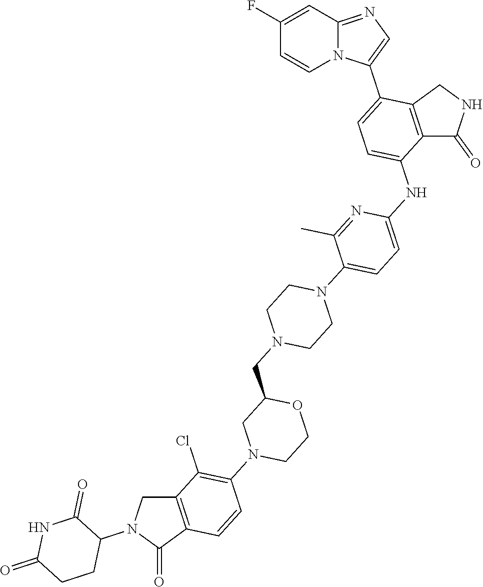 [SMILES: Cc1nc(Nc2ccc(-c3cnc4cc(F)ccn34)c3c2C(=O)NC3)ccc1N1CCN(C[C@@H]2CN(c3ccc4c(c3Cl)CN(C3CCC(=O)NC3=O)C4=O)CCO2)CC1] |
| 374 | 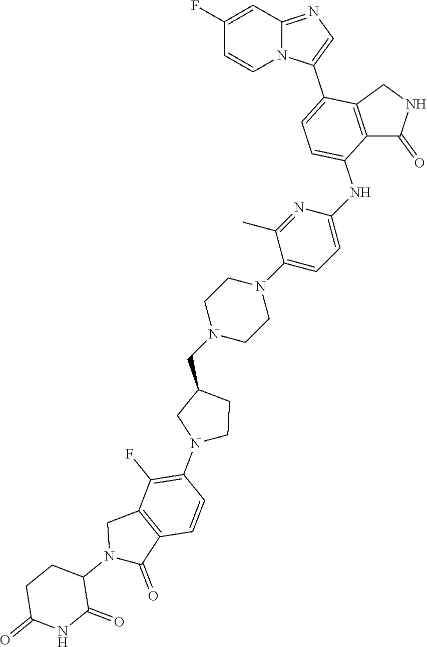 [SMILES: Cc1nc(Nc2ccc(-c3cnc4cc(F)ccn34)c3c2C(=O)NC3)ccc1N1CCN(C[C@H]2CCN(c3ccc4c(c3F)CN(C3CCC(=O)NC3=O)C4=O)C2)CC1] |
| 375 | 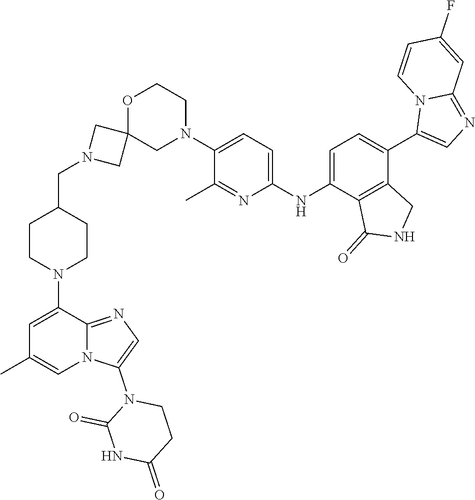 [SMILES: Cc1cc(N2CCC(CN3CC4(C3)CN(c3ccc(Nc5ccc(-c6cnc7cc(F)ccn67)c6c5C(=O)NC6)nc3C)CCO4)CC2)c2ncc(N3CCC(=O)NC3=O)n2c1] |
| 376 | 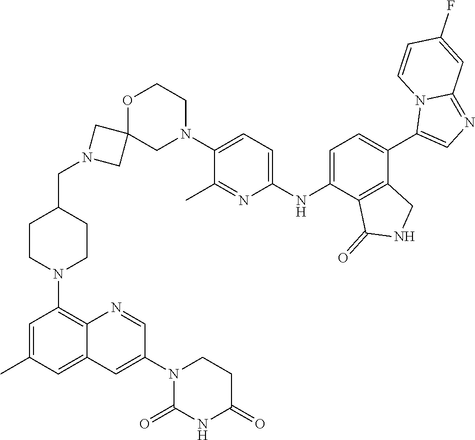 [SMILES: Cc1cc(N2CCC(CN3CC4(C3)CN(c3ccc(Nc5ccc(-c6cnc7cc(F)ccn67)c6c5C(=O)NC6)nc3C)CCO4)CC2)c2ncc(N3CCC(=O)NC3=O)cc2c1] |
| 377 | 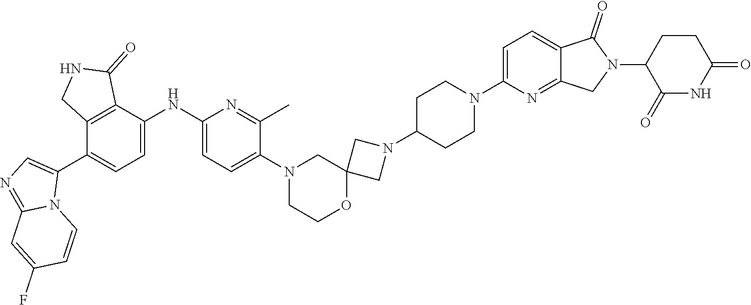 [SMILES: Cc1nc(Nc2ccc(-c3cnc4cc(F)ccn34)c3c2C(=O)NC3)ccc1N1CCOC2(C1)CN(C1CCN(c3ccc4c(n3)CN(C3CCC(=O)NC3=O)C4=O)CC1)C2] |
| 378 | 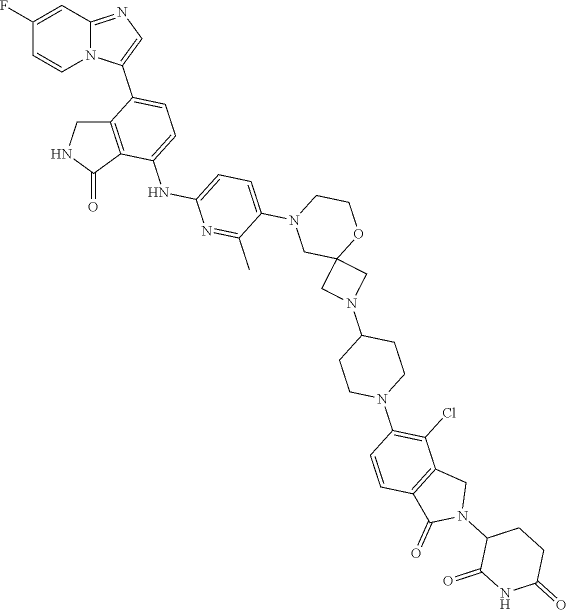 [SMILES: Cc1nc(Nc2ccc(-c3cnc4cc(F)ccn34)c3c2C(=O)NC3)ccc1N1CCOC2(C1)CN(C1CCN(c3ccc4c(c3Cl)CN(C3CCC(=O)NC3=O)C4=O)CC1)C2] |
| 379 | 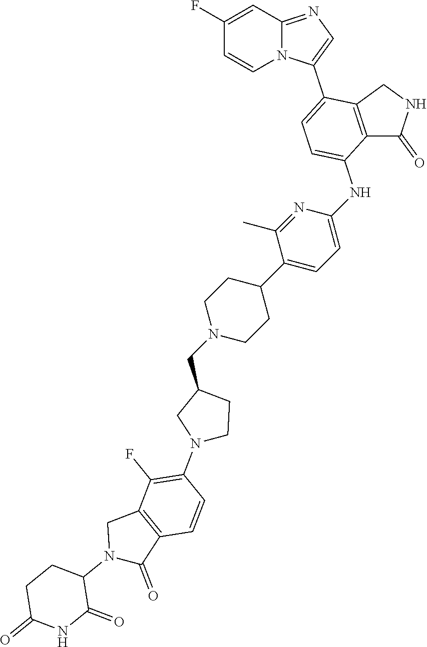 [SMILES: Cc1nc(Nc2ccc(-c3cnc4cc(F)ccn34)c3c2C(=O)NC3)ccc1C1CCN(C[C@H]2CCN(c3ccc4c(c3F)CN(C3CCC(=O)NC3=O)C4=O)C2)CC1] |
| 380 | 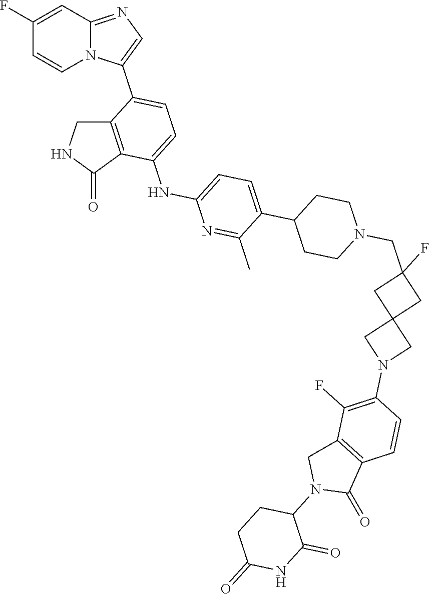 [SMILES: Cc1nc(Nc2ccc(-c3cnc4cc(F)ccn34)c3c2C(=O)NC3)ccc1C1CCN(CC2(F)CC3(CN(c4ccc5c(c4F)CN(C4CCC(=O)NC4=O)C5=O)C3)C2)CC1] |
| 381 | 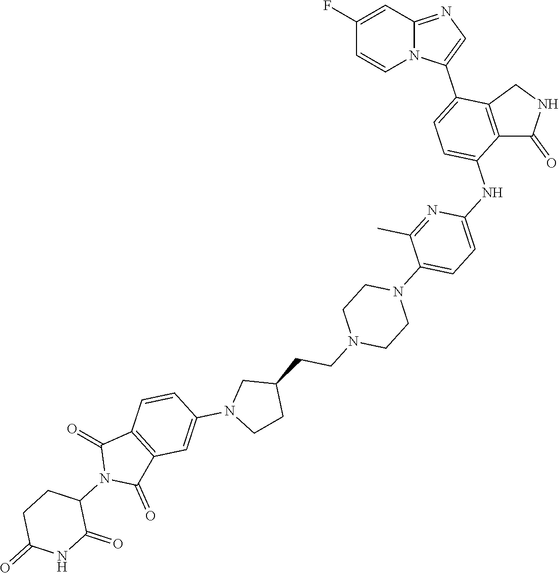 [SMILES: Cc1nc(Nc2ccc(-c3cnc4cc(F)ccn34)c3c2C(=O)NC3)ccc1N1CCN(CC[C@H]2CCN(c3ccc4c(c3)C(=O)N(C3CCC(=O)NC3=O)C4=O)C2)CC1] |
| 382 | 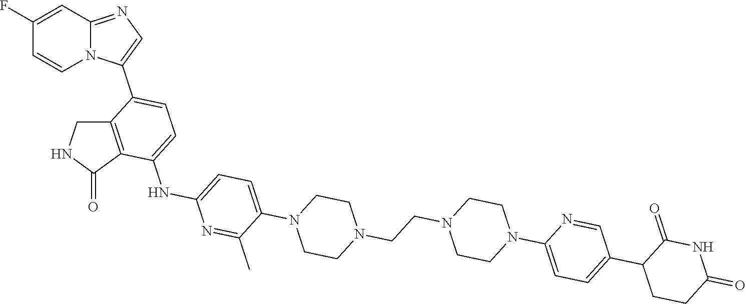 [SMILES: Cc1nc(Nc2ccc(-c3cnc4cc(F)ccn34)c3c2C(=O)NC3)ccc1N1CCN(CCN2CCN(c3ccc(C4CCC(=O)NC4=O)cn3)CC2)CC1] |
| 383 | 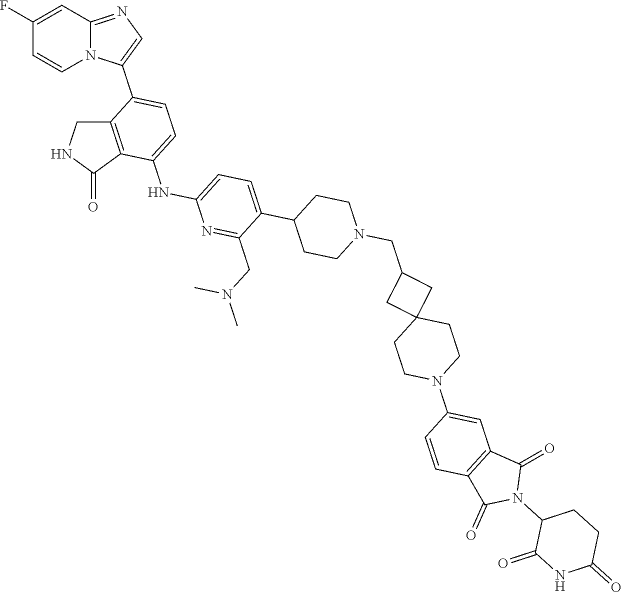 [SMILES: CN(C)Cc1nc(Nc2ccc(-c3cnc4cc(F)ccn34)c3c2C(=O)NC3)ccc1C1CCN(CC2CC3(CCN(c4ccc5c(c4)C(=O)N(C4CCC(=O)NC4=O)C5=O)CC3)C2)CC1] |
| 384 | 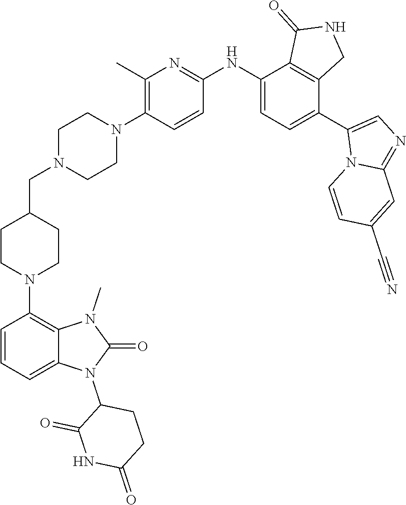 [SMILES: Cc1nc(Nc2ccc(-c3cnc4cc(C#N)ccn34)c3c2C(=O)NC3)ccc1N1CCN(CC2CCN(c3cccc4c3n(C)c(=O)n4C3CCC(=O)NC3=O)CC2)CC1] |
| 385 | 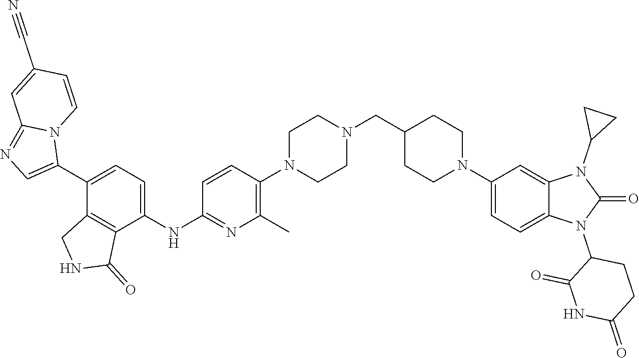 [SMILES: Cc1nc(Nc2ccc(-c3cnc4cc(C#N)ccn34)c3c2C(=O)NC3)ccc1N1CCN(CC2CCN(c3ccc4c(c3)n(C3CC3)c(=O)n4C3CCC(=O)NC3=O)CC2)CC1] |
| 386 | 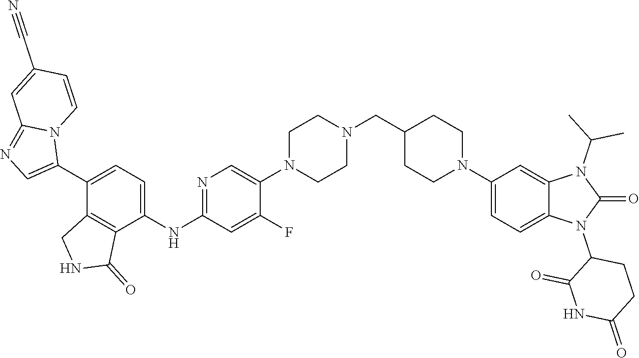 [SMILES: CC(C)n1c(=O)n(C2CCC(=O)NC2=O)c2ccc(N3CCC(CN4CCN(c5cnc(Nc6ccc(-c7cnc8cc(C#N)ccn78)c7c6C(=O)NC7)cc5F)CC4)CC3)cc21] |
| 387 | 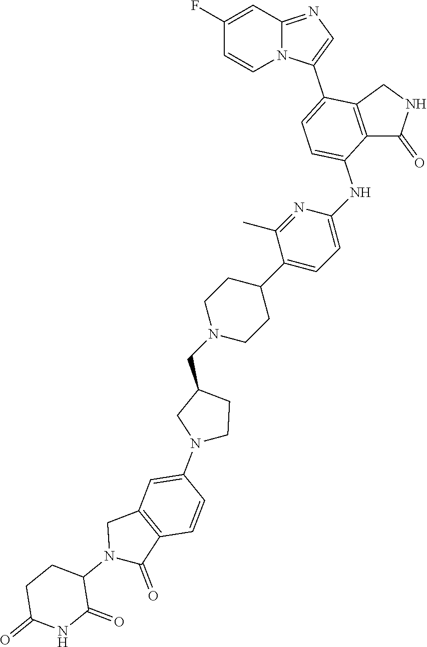 [SMILES: Cc1nc(Nc2ccc(-c3cnc4cc(F)ccn34)c3c2C(=O)NC3)ccc1C1CCN(C[C@H]2CCN(c3ccc4c(c3)CN(C3CCC(=O)NC3=O)C4=O)C2)CC1] |
| 388 | 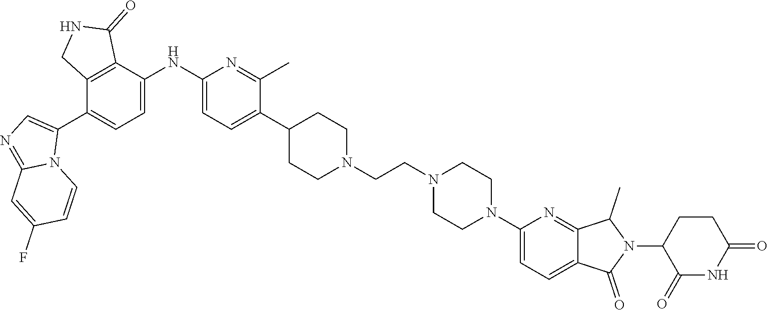 [SMILES: Cc1nc(Nc2ccc(-c3cnc4cc(F)ccn34)c3c2C(=O)NC3)ccc1C1CCN(CCN2CCN(c3ccc4c(n3)C(C)N(C3CCC(=O)NC3=O)C4=O)CC2)CC1] |
| 389 | 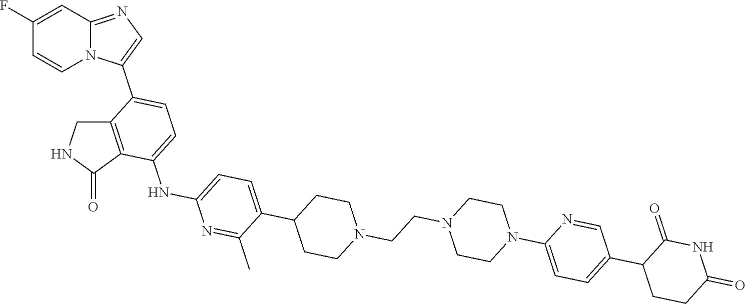 [SMILES: Cc1nc(Nc2ccc(-c3cnc4cc(F)ccn34)c3c2C(=O)NC3)ccc1C1CCN(CCN2CCN(c3ccc(C4CCC(=O)NC4=O)cn3)CC2)CC1] |
| 390 | 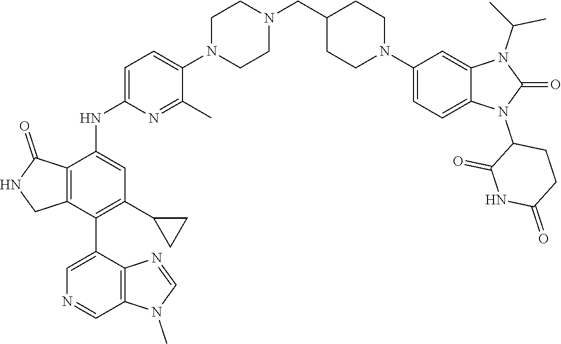 [SMILES: Cc1nc(Nc2cc(C3CC3)c(-c3cncc4c3ncn4C)c3c2C(=O)NC3)ccc1N1CCN(CC2CCN(c3ccc4c(c3)n(C(C)C)c(=O)n4C3CCC(=O)NC3=O)CC2)CC1] |
| 391 | 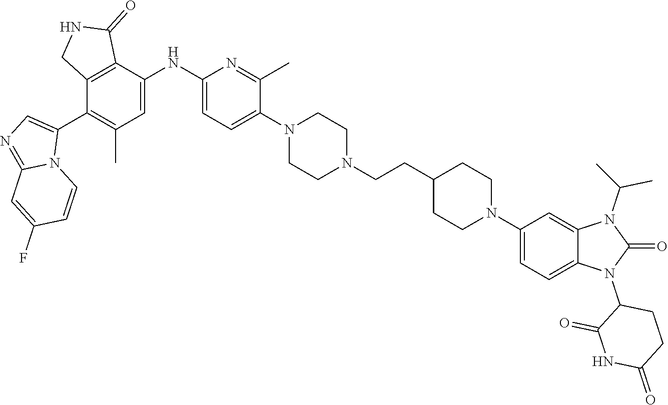 [SMILES: Cc1cc(Nc2ccc(N3CCN(CCC4CCN(c5ccc6c(c5)n(C(C)C)c(=O)n6C5CCC(=O)NC5=O)CC4)CC3)c(C)n2)c2c(c1-c1cnc3cc(F)ccn13)CNC2=O] |
| 392 | 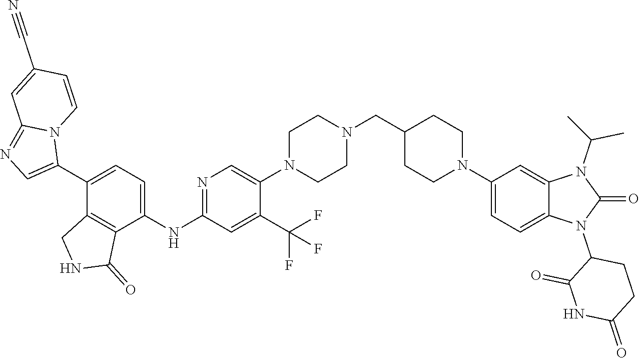 [SMILES: CC(C)n1c(=O)n(C2CCC(=O)NC2=O)c2ccc(N3CCC(CN4CCN(c5cnc(Nc6ccc(-c7cnc8cc(C#N)ccn78)c7c6C(=O)NC7)cc5C(F)(F)F)CC4)CC3)cc21] |
| 393 | 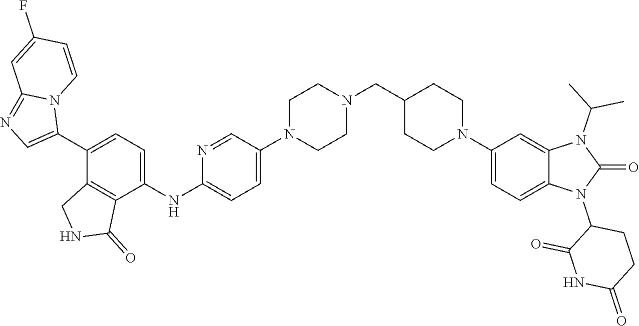 [SMILES: CC(C)n1c(=O)n(C2CCC(=O)NC2=O)c2ccc(N3CCC(CN4CCN(c5ccc(Nc6ccc(-c7cnc8cc(F)ccn78)c7c6C(=O)NC7)nc5)CC4)CC3)cc21] |
| 394 | 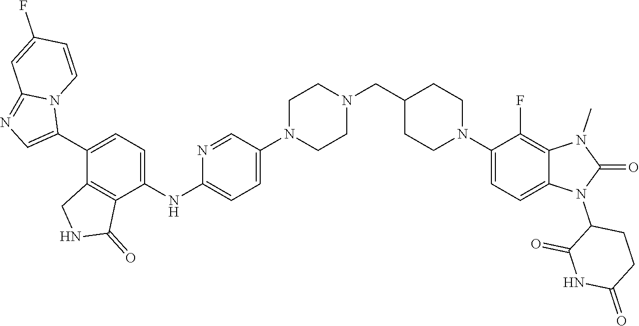 [SMILES: Cn1c(=O)n(C2CCC(=O)NC2=O)c2ccc(N3CCC(CN4CCN(c5ccc(Nc6ccc(-c7cnc8cc(F)ccn78)c7c6C(=O)NC7)nc5)CC4)CC3)c(F)c21] |
| 395 | 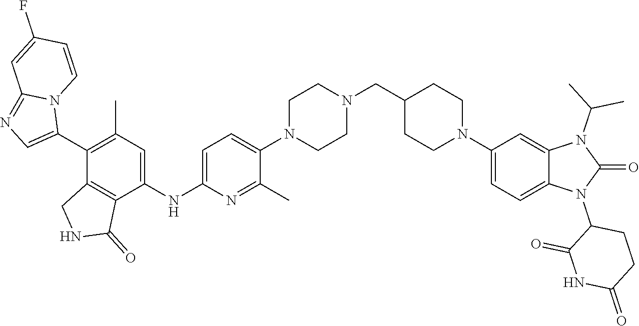 [SMILES: Cc1cc(Nc2ccc(N3CCN(CC4CCN(c5ccc6c(c5)n(C(C)C)c(=O)n6C5CCC(=O)NC5=O)CC4)CC3)c(C)n2)c2c(c1-c1cnc3cc(F)ccn13)CNC2=O] |
| 396 | 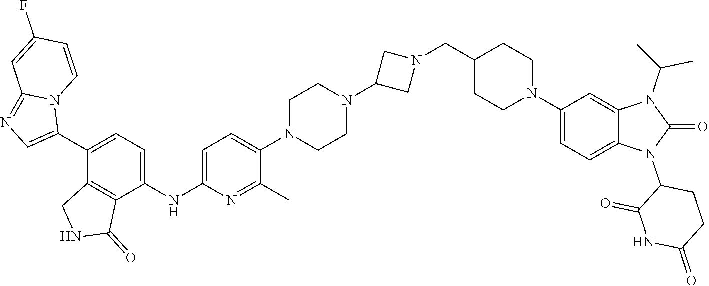 [SMILES: Cc1nc(Nc2ccc(-c3cnc4cc(F)ccn34)c3c2C(=O)NC3)ccc1N1CCN(C2CN(CC3CCN(c4ccc5c(c4)n(C(C)C)c(=O)n5C4CCC(=O)NC4=O)CC3)C2)CC1] |
| 397 | 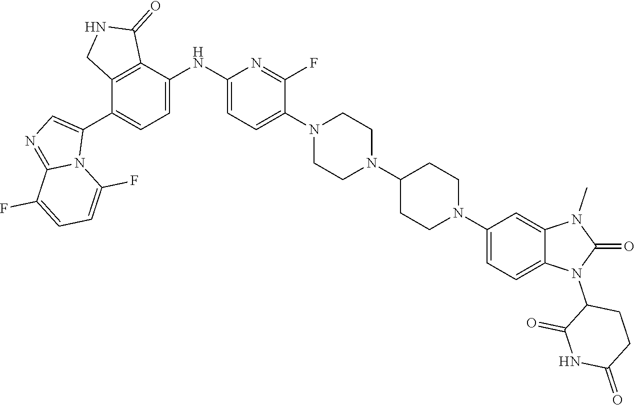 [SMILES: Cn1c(=O)n(C2CCC(=O)NC2=O)c2ccc(N3CCC(N4CCN(c5ccc(Nc6ccc(-c7cnc8c(F)ccc(F)n78)c7c6C(=O)NC7)nc5F)CC4)CC3)cc21] |
| 398 | 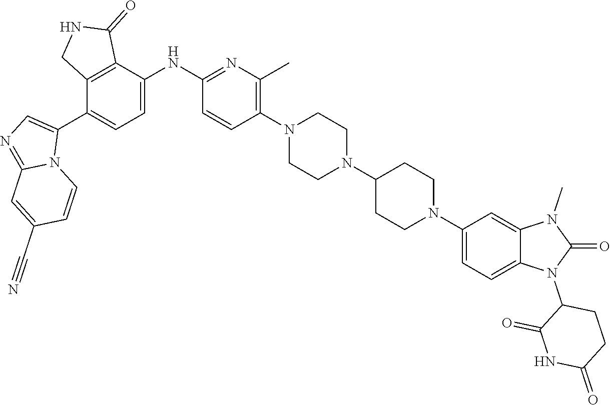 [SMILES: Cc1nc(Nc2ccc(-c3cnc4cc(C#N)ccn34)c3c2C(=O)NC3)ccc1N1CCN(C2CCN(c3ccc4c(c3)n(C)c(=O)n4C3CCC(=O)NC3=O)CC2)CC1] |
| 399 | 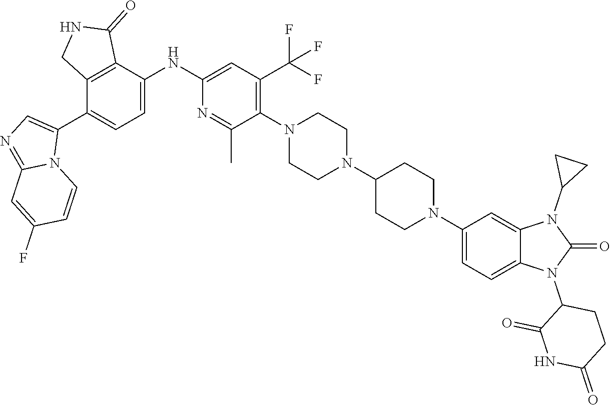 [SMILES: Cc1nc(Nc2ccc(-c3cnc4cc(F)ccn34)c3c2C(=O)NC3)cc(C(F)(F)F)c1N1CCN(C2CCN(c3ccc4c(c3)n(C3CC3)c(=O)n4C3CCC(=O)NC3=O)CC2)CC1] |
| 400 | 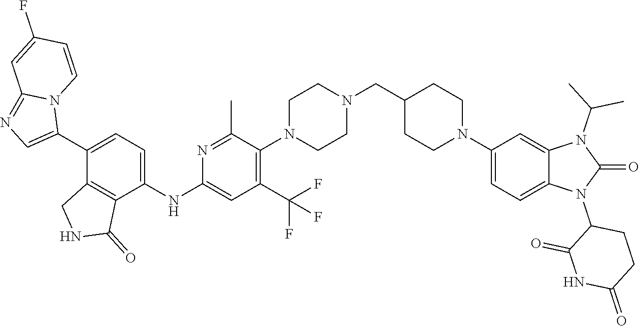 [SMILES: Cc1nc(Nc2ccc(-c3cnc4cc(F)ccn34)c3c2C(=O)NC3)cc(C(F)(F)F)c1N1CCN(CC2CCN(c3ccc4c(c3)n(C(C)C)c(=O)n4C3CCC(=O)NC3=O)CC2)CC1] |
| 401 | 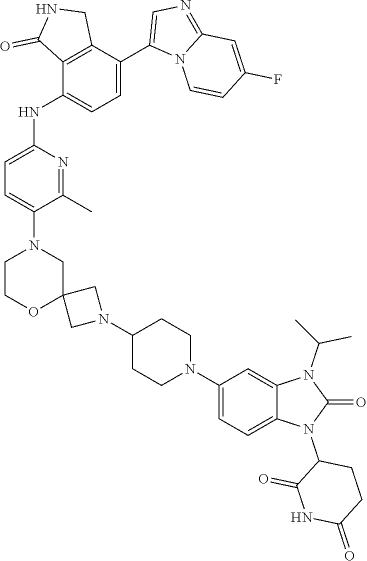 [SMILES: Cc1nc(Nc2ccc(-c3cnc4cc(F)ccn34)c3c2C(=O)NC3)ccc1N1CCOC2(C1)CN(C1CCN(c3ccc4c(c3)n(C(C)C)c(=O)n4C3CCC(=O)NC3=O)CC1)C2] |
| 402 | 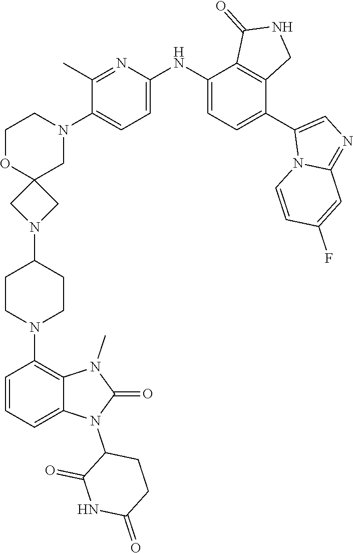 [SMILES: Cc1nc(Nc2ccc(-c3cnc4cc(F)ccn34)c3c2C(=O)NC3)ccc1N1CCOC2(C1)CN(C1CCN(c3cccc4c3n(C)c(=O)n4C3CCC(=O)NC3=O)CC1)C2] |
| 403 | 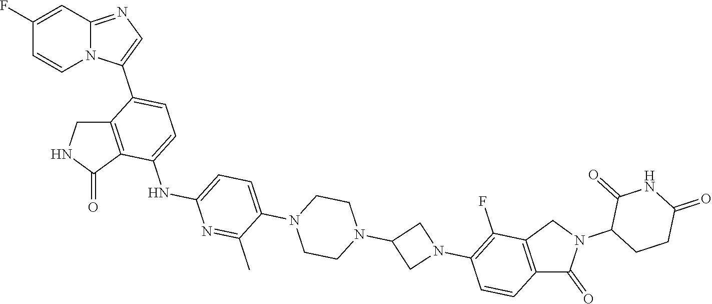 [SMILES: Cc1nc(Nc2ccc(-c3cnc4cc(F)ccn34)c3c2C(=O)NC3)ccc1N1CCN(C2CN(c3ccc4c(c3F)CN(C3CCC(=O)NC3=O)C4=O)C2)CC1] |
| 404 | 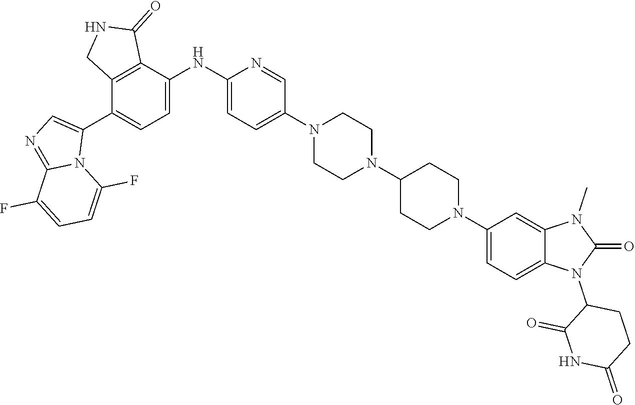 [SMILES: Cn1c(=O)n(C2CCC(=O)NC2=O)c2ccc(N3CCC(N4CCN(c5ccc(Nc6ccc(-c7cnc8c(F)ccc(F)n78)c7c6C(=O)NC7)nc5)CC4)CC3)cc21] |
| 405 | 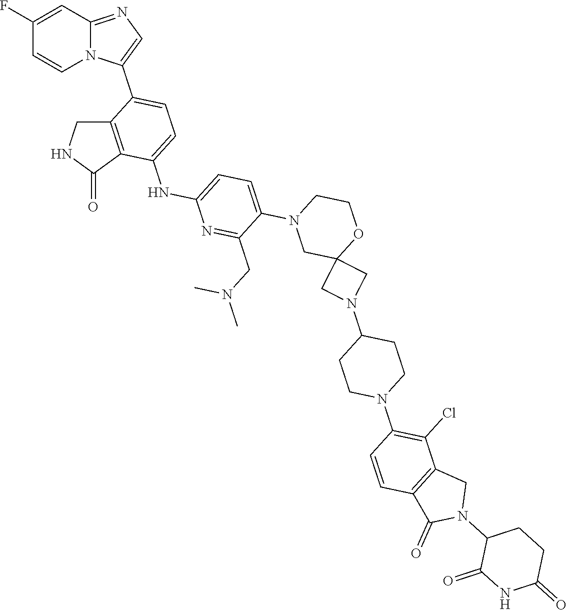 [SMILES: CN(C)Cc1nc(Nc2ccc(-c3cnc4cc(F)ccn34)c3c2C(=O)NC3)ccc1N1CCOC2(C1)CN(C1CCN(c3ccc4c(c3Cl)CN(C3CCC(=O)NC3=O)C4=O)CC1)C2] |
| 406 | 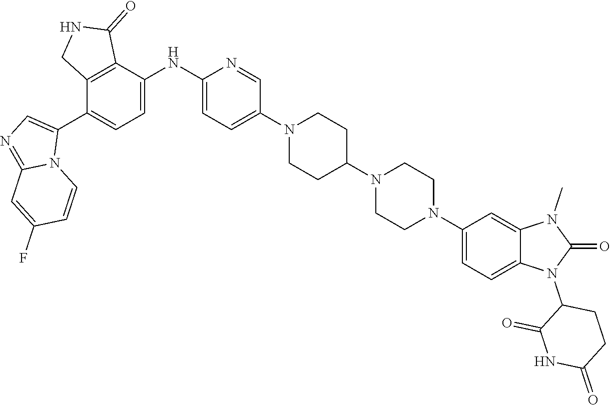 [SMILES: Cn1c(=O)n(C2CCC(=O)NC2=O)c2ccc(N3CCN(C4CCN(c5ccc(Nc6ccc(-c7cnc8cc(F)ccn78)c7c6C(=O)NC7)nc5)CC4)CC3)cc21] |
| 407 | 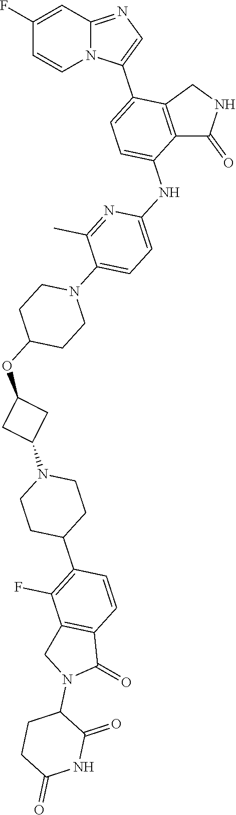 [SMILES: Cc1nc(Nc2ccc(-c3cnc4cc(F)ccn34)c3c2C(=O)NC3)ccc1N1CCC(O[C@H]2C[C@H](N3CCC(c4ccc5c(c4F)CN(C4CCC(=O)NC4=O)C5=O)CC3)C2)CC1] |
| 408 | 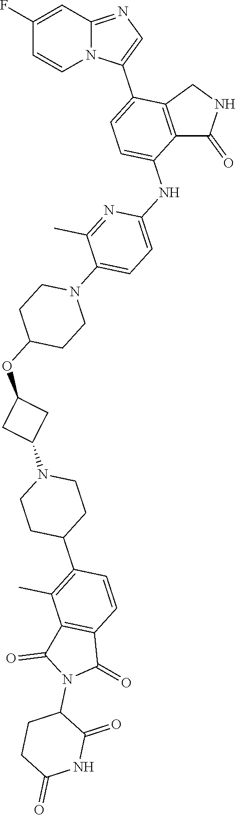 [SMILES: Cc1nc(Nc2ccc(-c3cnc4cc(F)ccn34)c3c2C(=O)NC3)ccc1N1CCC(O[C@H]2C[C@H](N3CCC(c4ccc5c(c4C)C(=O)N(C4CCC(=O)NC4=O)C5=O)CC3)C2)CC1] |
| 409 | 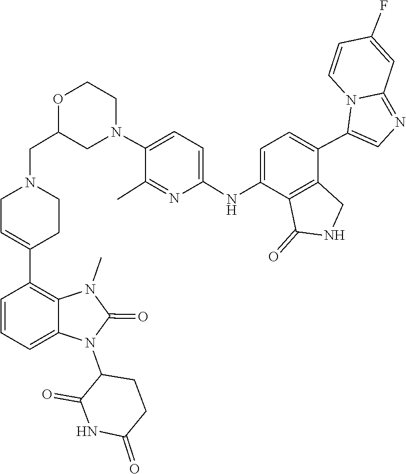 [SMILES: Cc1nc(Nc2ccc(-c3cnc4cc(F)ccn34)c3c2C(=O)NC3)ccc1N1CCOC(CN2CC=C(c3cccc4c3n(C)c(=O)n4C3CCC(=O)NC3=O)CC2)C1] |
| 410 | 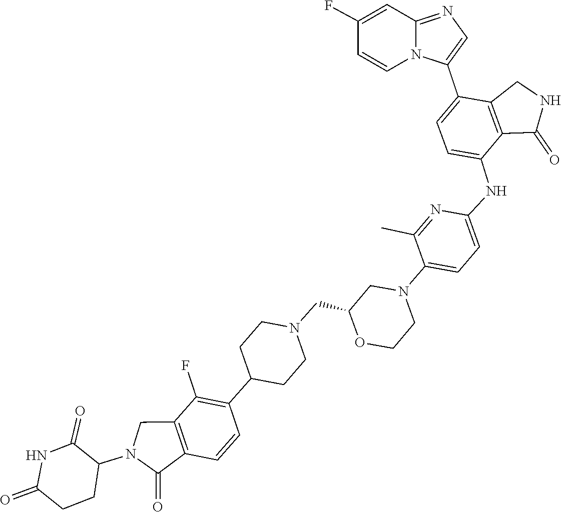 [SMILES: Cc1nc(Nc2ccc(-c3cnc4cc(F)ccn34)c3c2C(=O)NC3)ccc1N1CCO[C@H](CN2CCC(c3ccc4c(c3F)CN(C3CCC(=O)NC3=O)C4=O)CC2)C1] |
| 411 | 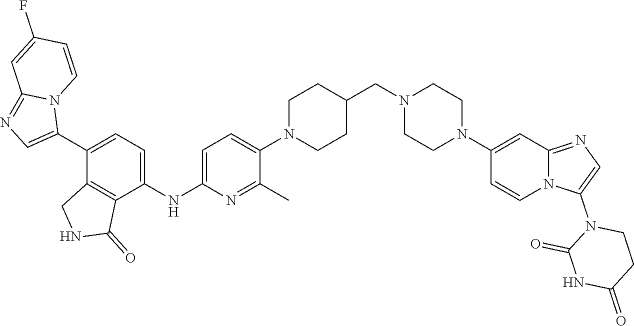 [SMILES: Cc1nc(Nc2ccc(-c3cnc4cc(F)ccn34)c3c2C(=O)NC3)ccc1N1CCC(CN2CCN(c3ccn4c(N5CCC(=O)NC5=O)cnc4c3)CC2)CC1] |
| 412 | 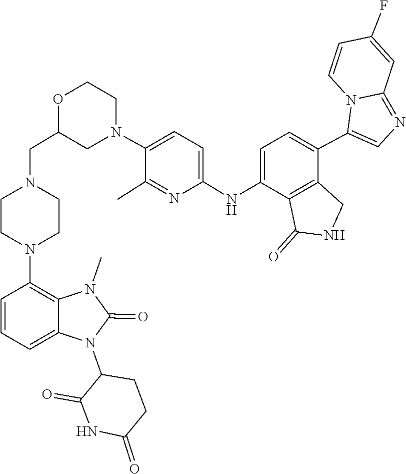 [SMILES: Cc1nc(Nc2ccc(-c3cnc4cc(F)ccn34)c3c2C(=O)NC3)ccc1N1CCOC(CN2CCN(c3cccc4c3n(C)c(=O)n4C3CCC(=O)NC3=O)CC2)C1] |
| 413 | 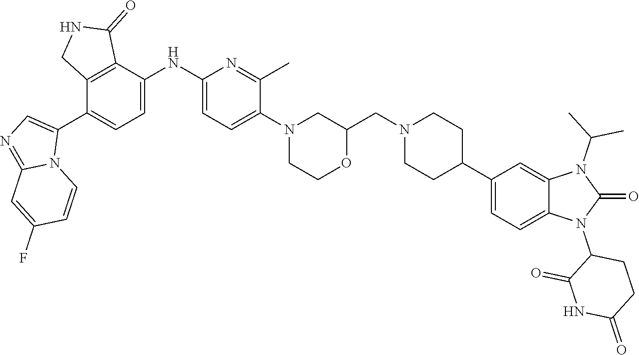 [SMILES: Cc1nc(Nc2ccc(-c3cnc4cc(F)ccn34)c3c2C(=O)NC3)ccc1N1CCOC(CN2CCC(c3ccc4c(c3)n(C(C)C)c(=O)n4C3CCC(=O)NC3=O)CC2)C1] |
| 414 | 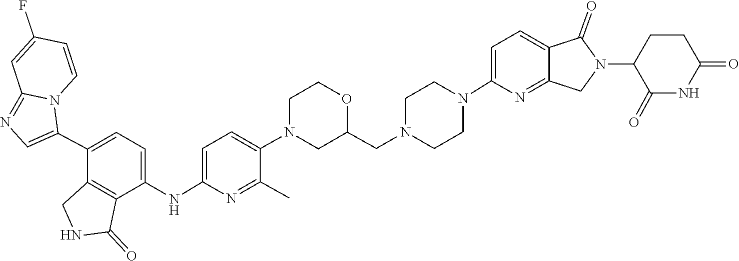 [SMILES: Cc1nc(Nc2ccc(-c3cnc4cc(F)ccn34)c3c2C(=O)NC3)ccc1N1CCOC(CN2CCN(c3ccc4c(n3)CN(C3CCC(=O)NC3=O)C4=O)CC2)C1] |
| 415 | 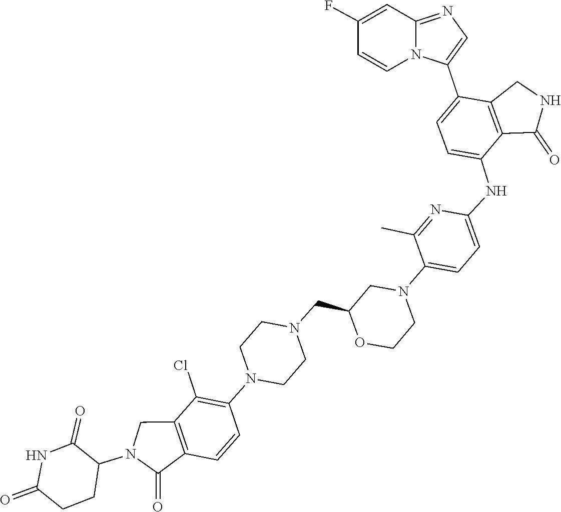 [SMILES: Cc1nc(Nc2ccc(-c3cnc4cc(F)ccn34)c3c2C(=O)NC3)ccc1N1CCO[C@@H](CN2CCN(c3ccc4c(c3Cl)CN(C3CCC(=O)NC3=O)C4=O)CC2)C1] |
| 416 | 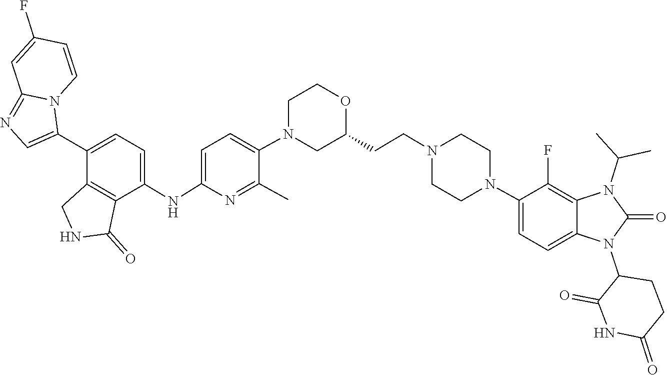 [SMILES: Cc1nc(Nc2ccc(-c3cnc4cc(F)ccn34)c3c2C(=O)NC3)ccc1N1CCO[C@H](CCN2CCN(c3ccc4c(c3F)n(C(C)C)c(=O)n4C3CCC(=O)NC3=O)CC2)C1] |
| 417 | 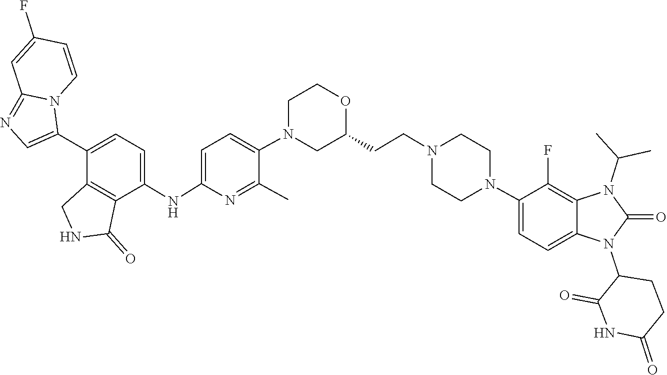 [SMILES: Cc1nc(Nc2ccc(-c3cnc4cc(F)ccn34)c3c2C(=O)NC3)ccc1N1CCO[C@H](CCN2CCN(c3ccc4c(c3F)n(C(C)C)c(=O)n4C3CCC(=O)NC3=O)CC2)C1] |
| 418 | 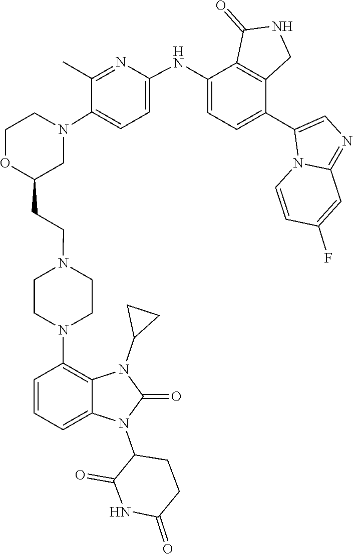 [SMILES: Cc1nc(Nc2ccc(-c3cnc4cc(F)ccn34)c3c2C(=O)NC3)ccc1N1CCO[C@H](CCN2CCN(c3cccc4c3n(C3CC3)c(=O)n4C3CCC(=O)NC3=O)CC2)C1] |
| 419 | 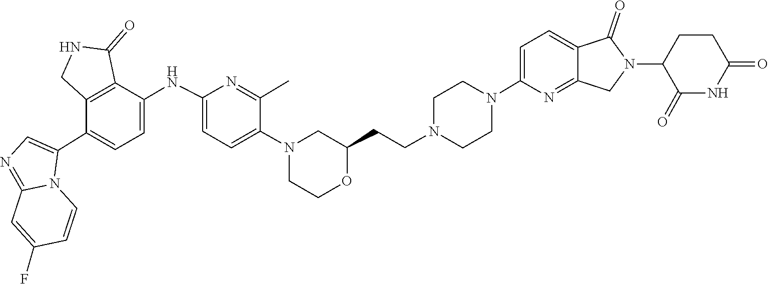 [SMILES: Cc1nc(Nc2ccc(-c3cnc4cc(F)ccn34)c3c2C(=O)NC3)ccc1N1CCO[C@H](CCN2CCN(c3ccc4c(n3)CN(C3CCC(=O)NC3=O)C4=O)CC2)C1] |
| 420 | 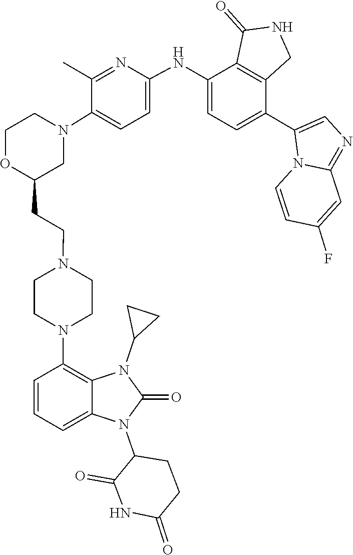 [SMILES: Cc1nc(Nc2ccc(-c3cnc4cc(F)ccn34)c3c2C(=O)NC3)ccc1N1CCO[C@H](CCN2CCN(c3cccc4c3n(C3CC3)c(=O)n4C3CCC(=O)NC3=O)CC2)C1] |
| 421 | 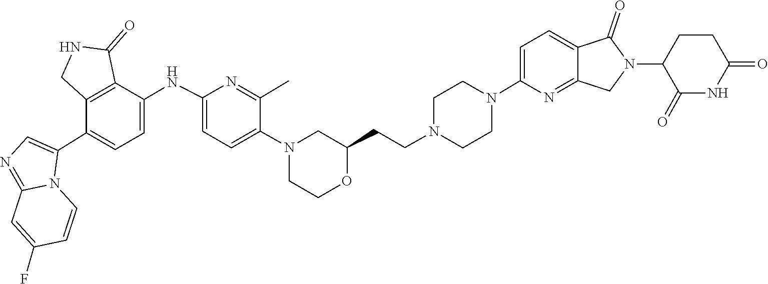 [SMILES: Cc1nc(Nc2ccc(-c3cnc4cc(F)ccn34)c3c2C(=O)NC3)ccc1N1CCO[C@H](CCN2CCN(c3ccc4c(n3)CN(C3CCC(=O)NC3=O)C4=O)CC2)C1] |
| 422 | 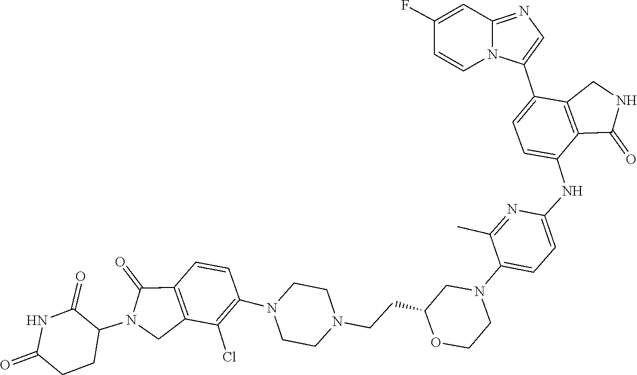 [SMILES: Cc1nc(Nc2ccc(-c3cnc4cc(F)ccn34)c3c2C(=O)NC3)ccc1N1CCO[C@H](CCN2CCN(c3ccc4c(c3Cl)CN(C3CCC(=O)NC3=O)C4=O)CC2)C1] |
| 423 | 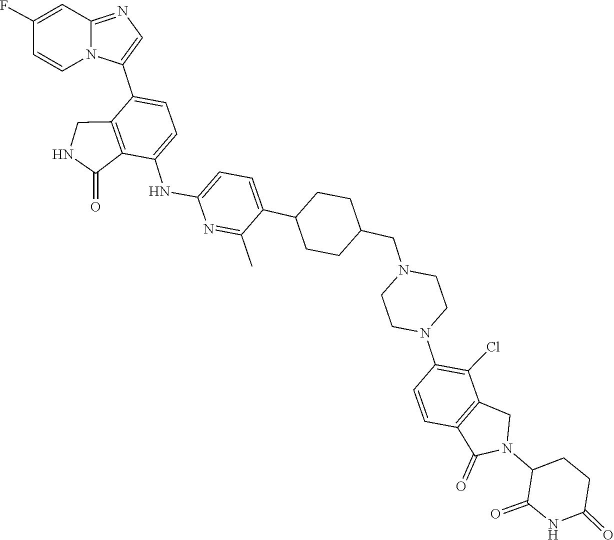 [SMILES: Cc1nc(Nc2ccc(-c3cnc4cc(F)ccn34)c3c2C(=O)NC3)ccc1C1CCC(CN2CCN(c3ccc4c(c3Cl)CN(C3CCC(=O)NC3=O)C4=O)CC2)CC1] |
| 424 | 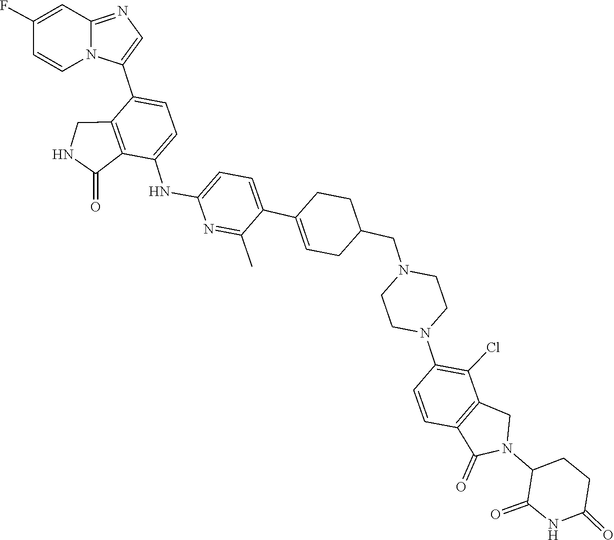 [SMILES: Cc1nc(Nc2ccc(-c3cnc4cc(F)ccn34)c3c2C(=O)NC3)ccc1C1=CCC(CN2CCN(c3ccc4c(c3Cl)CN(C3CCC(=O)NC3=O)C4=O)CC2)CC1] |
| 425 | 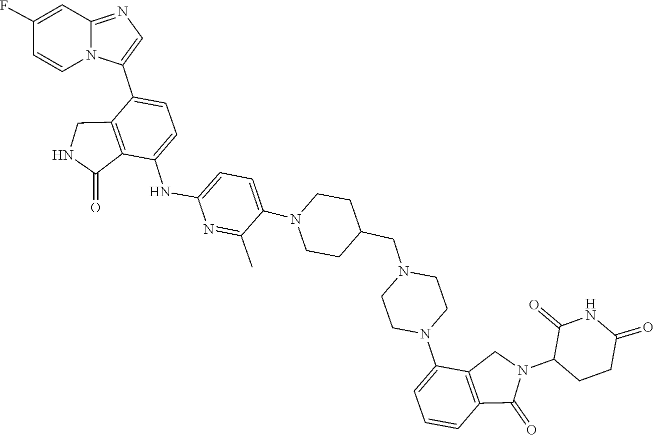 [SMILES: Cc1nc(Nc2ccc(-c3cnc4cc(F)ccn34)c3c2C(=O)NC3)ccc1N1CCC(CN2CCN(c3cccc4c3CN(C3CCC(=O)NC3=O)C4=O)CC2)CC1] |
| 426 | 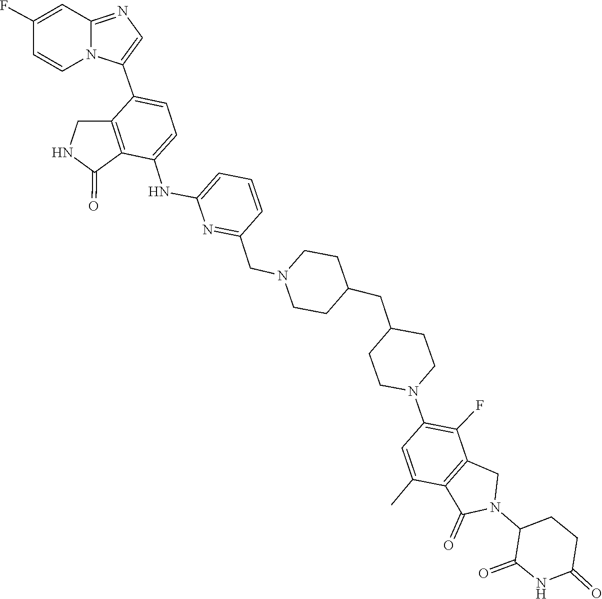 [SMILES: Cc1cc(N2CCC(CC3CCN(Cc4cccc(Nc5ccc(-c6cnc7cc(F)ccn67)c6c5C(=O)NC6)n4)CC3)CC2)c(F)c2c1C(=O)N(C1CCC(=O)NC1=O)C2] |
| 427 | 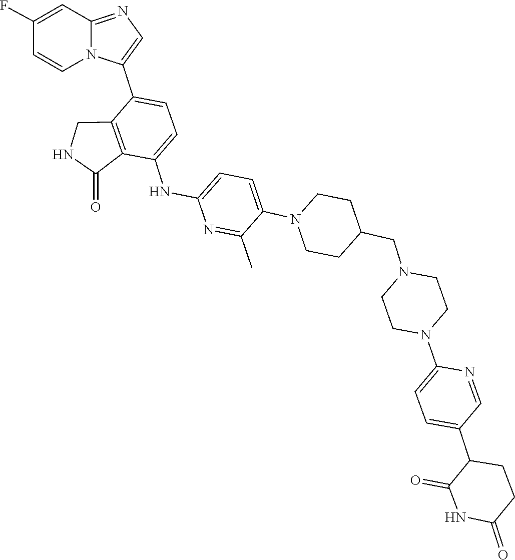 [SMILES: Cc1nc(Nc2ccc(-c3cnc4cc(F)ccn34)c3c2C(=O)NC3)ccc1N1CCC(CN2CCN(c3ccc(C4CCC(=O)NC4=O)cn3)CC2)CC1] |
| 428 | 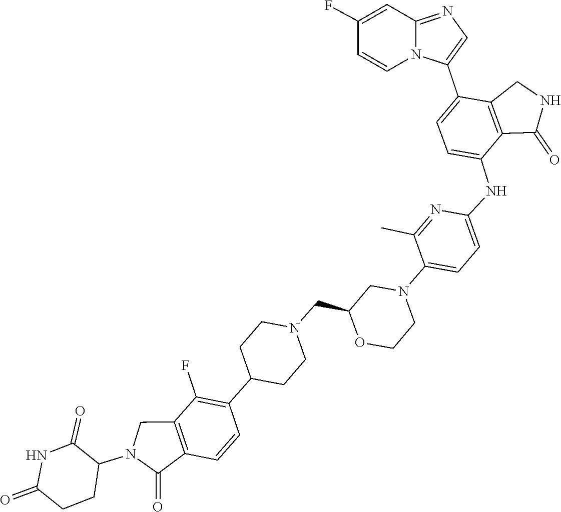 [SMILES: Cc1nc(Nc2ccc(-c3cnc4cc(F)ccn34)c3c2C(=O)NC3)ccc1N1CCO[C@@H](CN2CCC(c3ccc4c(c3F)CN(C3CCC(=O)NC3=O)C4=O)CC2)C1] |
| 429 | 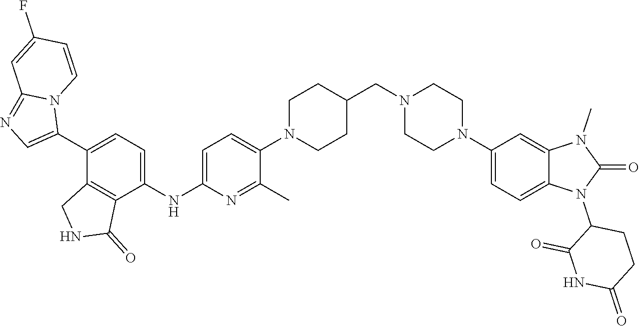 [SMILES: Cc1nc(Nc2ccc(-c3cnc4cc(F)ccn34)c3c2C(=O)NC3)ccc1N1CCC(CN2CCN(c3ccc4c(c3)n(C)c(=O)n4C3CCC(=O)NC3=O)CC2)CC1] |
| 430 | 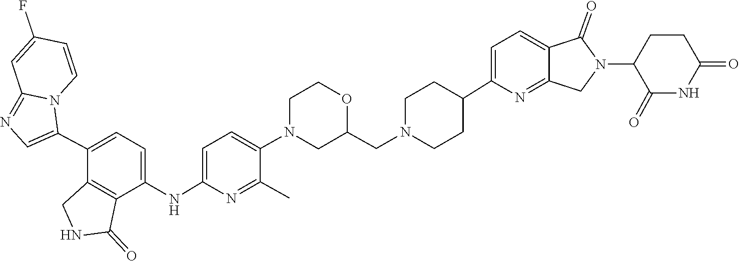 [SMILES: Cc1nc(Nc2ccc(-c3cnc4cc(F)ccn34)c3c2C(=O)NC3)ccc1N1CCOC(CN2CCC(c3ccc4c(n3)CN(C3CCC(=O)NC3=O)C4=O)CC2)C1] |
| 431 | 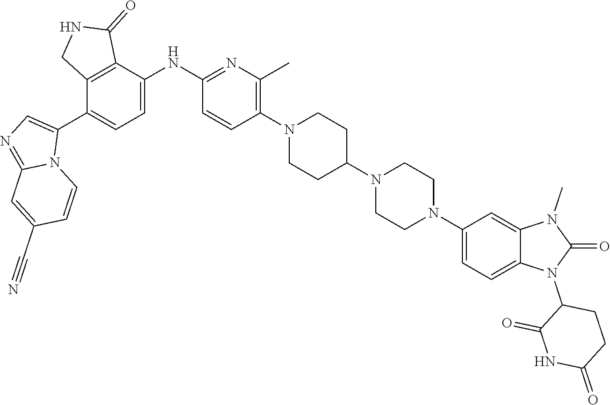 [SMILES: Cc1nc(Nc2ccc(-c3cnc4cc(C#N)ccn34)c3c2C(=O)NC3)ccc1N1CCC(N2CCN(c3ccc4c(c3)n(C)c(=O)n4C3CCC(=O)NC3=O)CC2)CC1] |
| 432 | 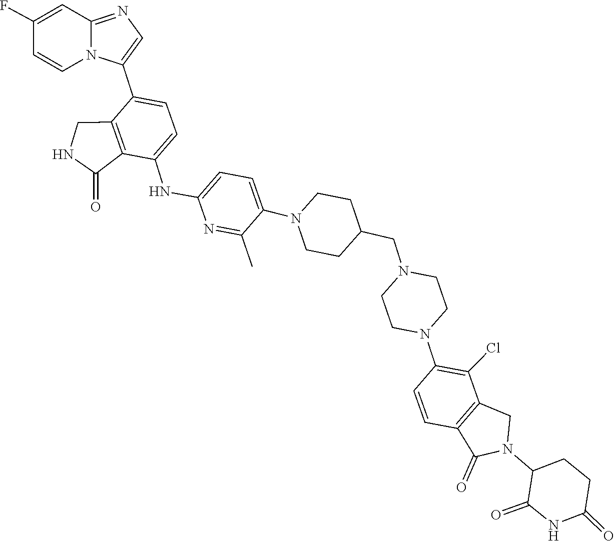 [SMILES: Cc1nc(Nc2ccc(-c3cnc4cc(F)ccn34)c3c2C(=O)NC3)ccc1N1CCC(CN2CCN(c3ccc4c(c3Cl)CN(C3CCC(=O)NC3=O)C4=O)CC2)CC1] |
| 433 | 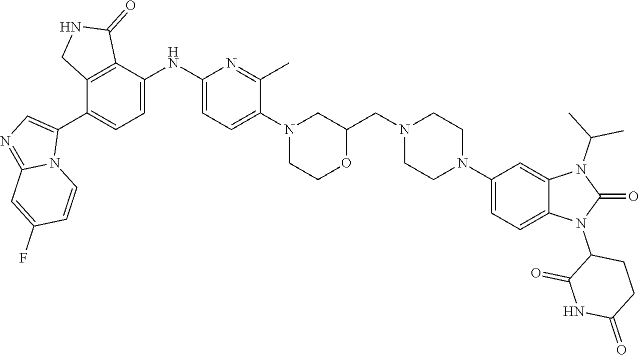 [SMILES: Cc1nc(Nc2ccc(-c3cnc4cc(F)ccn34)c3c2C(=O)NC3)ccc1N1CCOC(CN2CCN(c3ccc4c(c3)n(C(C)C)c(=O)n4C3CCC(=O)NC3=O)CC2)C1] |
| 434 | 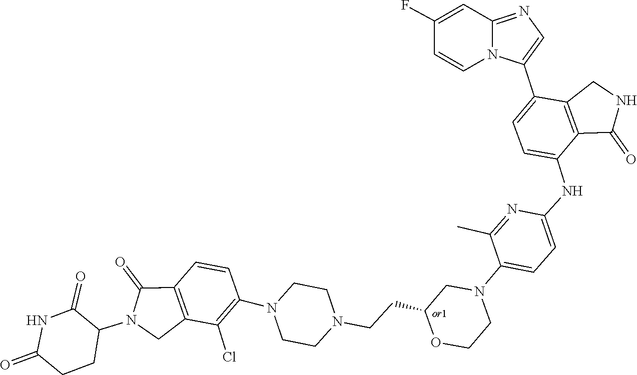 [SMILES: Cc1nc(Nc2ccc(-c3cnc4cc(F)ccn34)c3c2C(=O)NC3)ccc1N1CCO[C@H](CCN2CCN(c3ccc4c(c3Cl)CN(C3CCC(=O)NC3=O)C4=O)CC2)C1] |
| 435 | 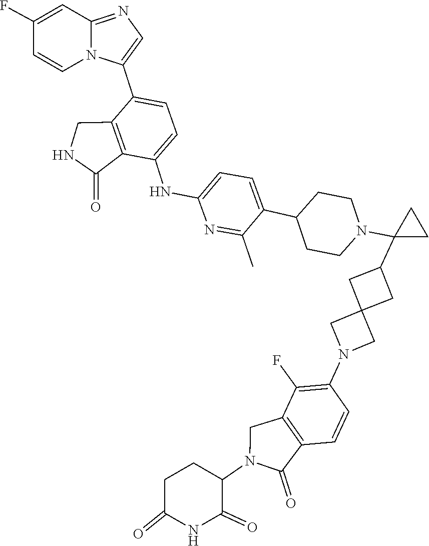 [SMILES: Cc1nc(Nc2ccc(-c3cnc4cc(F)ccn34)c3c2C(=O)NC3)ccc1C1CCN(C2(C3CC4(C3)CN(c3ccc5c(c3F)CN(C3CCC(=O)NC3=O)C5=O)C4)CC2)CC1] |
| 436 | 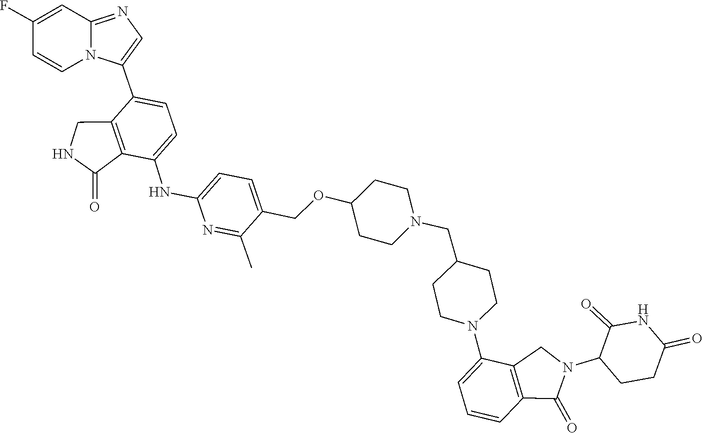 [SMILES: Cc1nc(Nc2ccc(-c3cnc4cc(F)ccn34)c3c2C(=O)NC3)ccc1COC1CCN(CC2CCN(c3cccc4c3CN(C3CCC(=O)NC3=O)C4=O)CC2)CC1] |
| 437 | 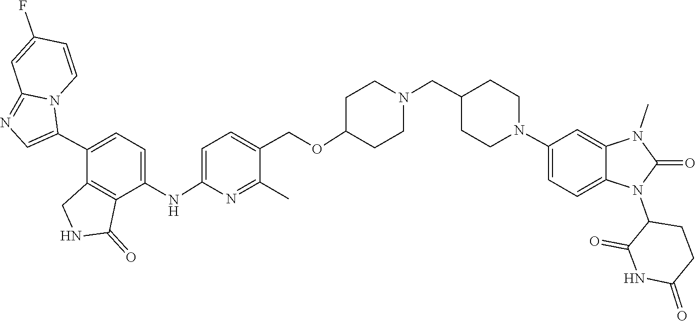 [SMILES: Cc1nc(Nc2ccc(-c3cnc4cc(F)ccn34)c3c2C(=O)NC3)ccc1COC1CCN(CC2CCN(c3ccc4c(c3)n(C)c(=O)n4C3CCC(=O)NC3=O)CC2)CC1] |
| 438 | 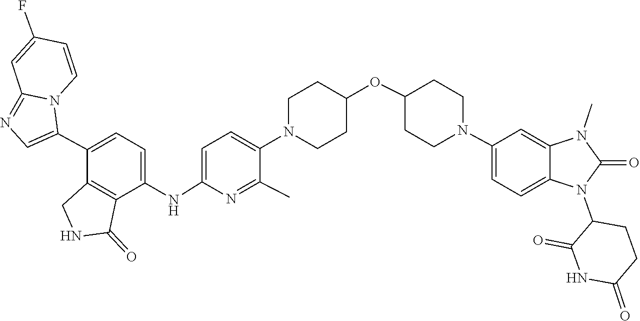 [SMILES: Cc1nc(Nc2ccc(-c3cnc4cc(F)ccn34)c3c2C(=O)NC3)ccc1N1CCC(OC2CCN(c3ccc4c(c3)n(C)c(=O)n4C3CCC(=O)NC3=O)CC2)CC1] |
| 439 | 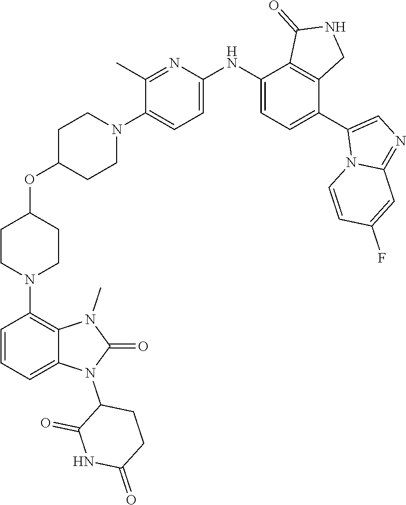 [SMILES: Cc1nc(Nc2ccc(-c3cnc4cc(F)ccn34)c3c2C(=O)NC3)ccc1N1CCC(OC2CCN(c3cccc4c3n(C)c(=O)n4C3CCC(=O)NC3=O)CC2)CC1] |
| 440 | 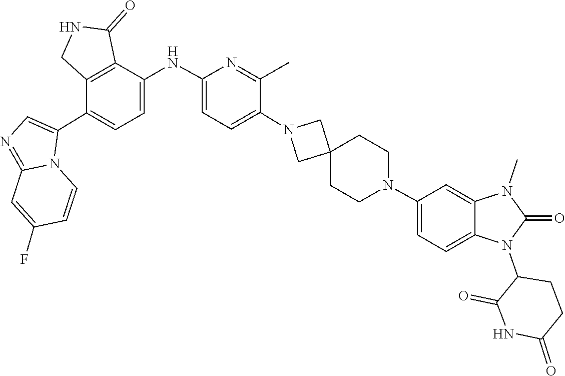 [SMILES: Cc1nc(Nc2ccc(-c3cnc4cc(F)ccn34)c3c2C(=O)NC3)ccc1N1CC2(CCN(c3ccc4c(c3)n(C)c(=O)n4C3CCC(=O)NC3=O)CC2)C1] |
| 441 | 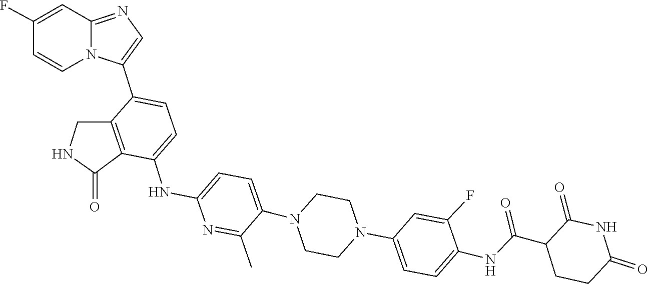 [SMILES: Cc1nc(Nc2ccc(-c3cnc4cc(F)ccn34)c3c2C(=O)NC3)ccc1N1CCN(c2ccc(NC(=O)C3CCC(=O)NC3=O)c(F)c2)CC1] |
| 442 | 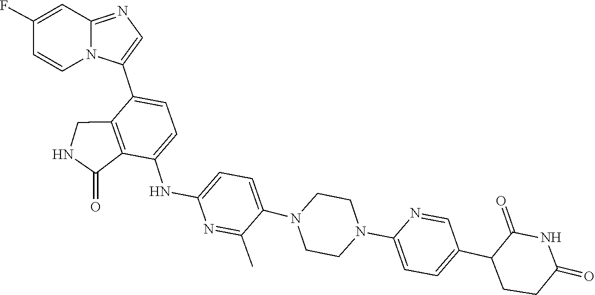 [SMILES: Cc1nc(Nc2ccc(-c3cnc4cc(F)ccn34)c3c2C(=O)NC3)ccc1N1CCN(c2ccc(C3CCC(=O)NC3=O)cn2)CC1] |
| 443 | 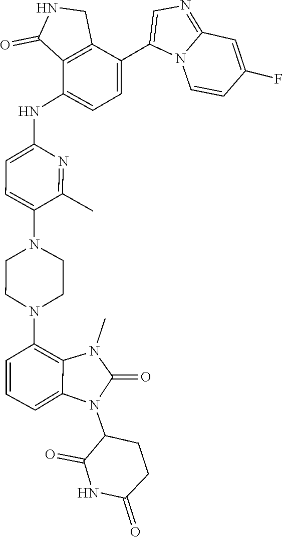 [SMILES: Cc1nc(Nc2ccc(-c3cnc4cc(F)ccn34)c3c2C(=O)NC3)ccc1N1CCN(c2cccc3c2n(C)c(=O)n3C2CCC(=O)NC2=O)CC1] |
| 444 | 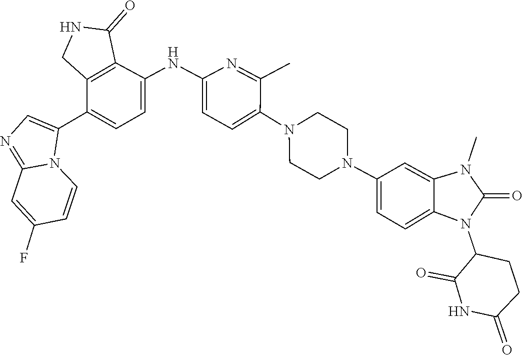 [SMILES: Cc1nc(Nc2ccc(-c3cnc4cc(F)ccn34)c3c2C(=O)NC3)ccc1N1CCN(c2ccc3c(c2)n(C)c(=O)n3C2CCC(=O)NC2=O)CC1] |
| 445 | 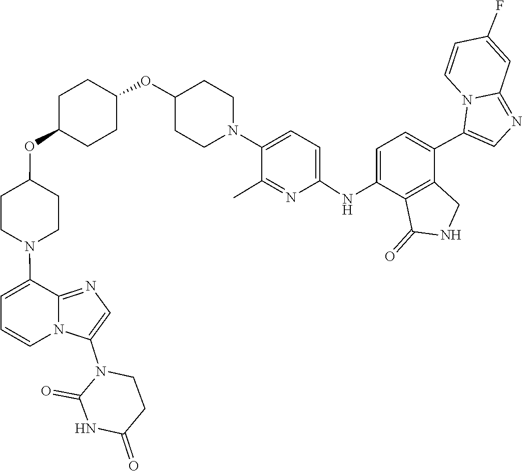 [SMILES: Cc1nc(Nc2ccc(-c3cnc4cc(F)ccn34)c3c2C(=O)NC3)ccc1N1CCC(O[C@H]2CC[C@H](OC3CCN(c4cccn5c(N6CCC(=O)NC6=O)cnc45)CC3)CC2)CC1] |
| 446 | 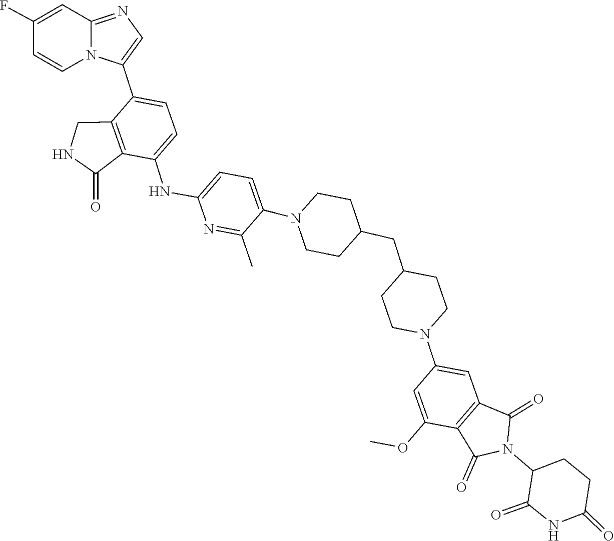 [SMILES: COc1cc(N2CCC(CC3CCN(c4ccc(Nc5ccc(-c6cnc7cc(F)ccn67)c6c5C(=O)NC6)nc4C)CC3)CC2)cc2c1C(=O)N(C1CCC(=O)NC1=O)C2=O] |
| 447 | 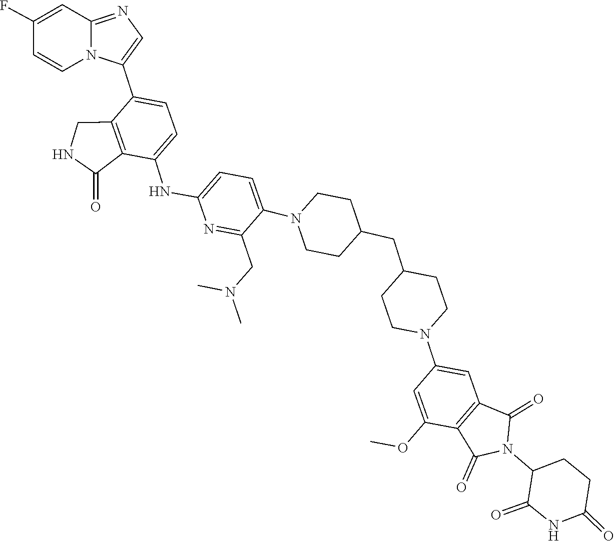 [SMILES: COc1cc(N2CCC(CC3CCN(c4ccc(Nc5ccc(-c6cnc7cc(F)ccn67)c6c5C(=O)NC6)nc4CN(C)C)CC3)CC2)cc2c1C(=O)N(C1CCC(=O)NC1=O)C2=O] |
| 448 | 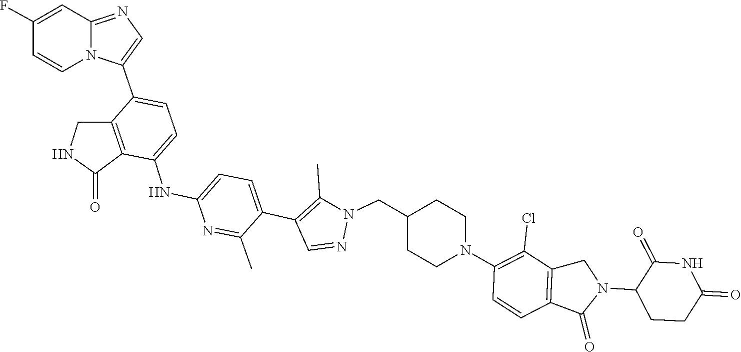 [SMILES: Cc1nc(Nc2ccc(-c3cnc4cc(F)ccn34)c3c2C(=O)NC3)ccc1-c1cnn(CC2CCN(c3ccc4c(c3Cl)CN(C3CCC(=O)NC3=O)C4=O)CC2)c1C] |
| 449 | 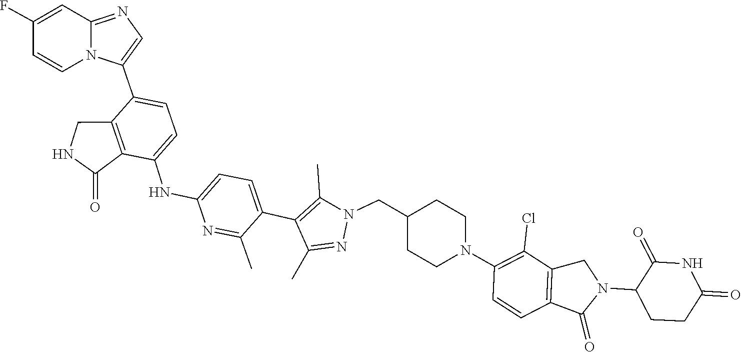 [SMILES: Cc1nc(Nc2ccc(-c3cnc4cc(F)ccn34)c3c2C(=O)NC3)ccc1-c1c(C)nn(CC2CCN(c3ccc4c(c3Cl)CN(C3CCC(=O)NC3=O)C4=O)CC2)c1C] |
| 450 | 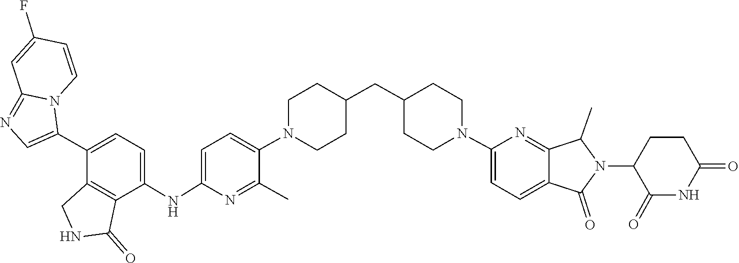 [SMILES: Cc1nc(Nc2ccc(-c3cnc4cc(F)ccn34)c3c2C(=O)NC3)ccc1N1CCC(CC2CCN(c3ccc4c(n3)C(C)N(C3CCC(=O)NC3=O)C4=O)CC2)CC1] |
| 451 | 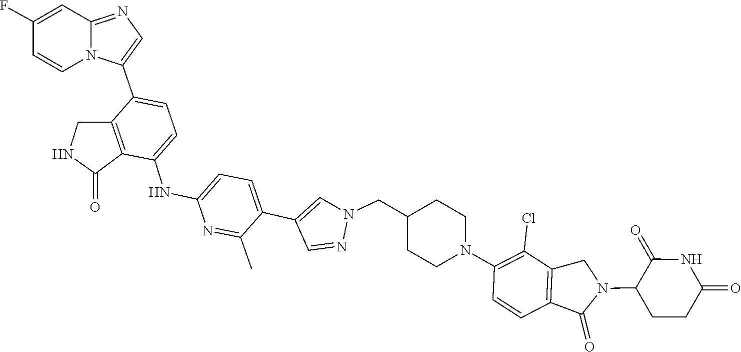 [SMILES: Cc1nc(Nc2ccc(-c3cnc4cc(F)ccn34)c3c2C(=O)NC3)ccc1-c1cnn(CC2CCN(c3ccc4c(c3Cl)CN(C3CCC(=O)NC3=O)C4=O)CC2)c1] |
| 452 | 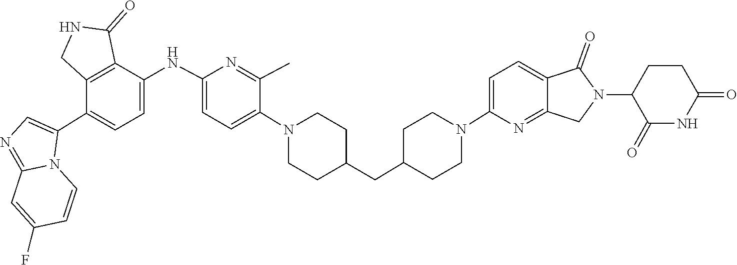 [SMILES: Cc1nc(Nc2ccc(-c3cnc4cc(F)ccn34)c3c2C(=O)NC3)ccc1N1CCC(CC2CCN(c3ccc4c(n3)CN(C3CCC(=O)NC3=O)C4=O)CC2)CC1] |
| 453 | 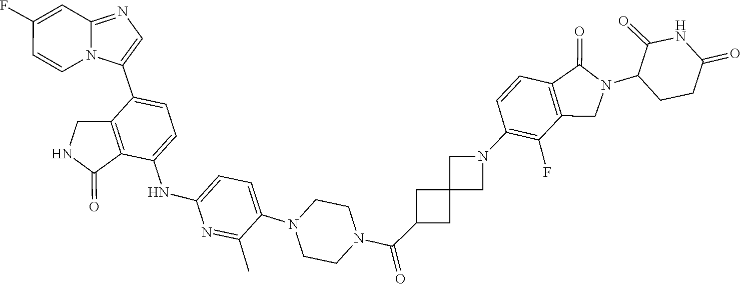 [SMILES: Cc1nc(Nc2ccc(-c3cnc4cc(F)ccn34)c3c2C(=O)NC3)ccc1N1CCN(C(=O)C2CC3(C2)CN(c2ccc4c(c2F)CN(C2CCC(=O)NC2=O)C4=O)C3)CC1] |
| 454 | 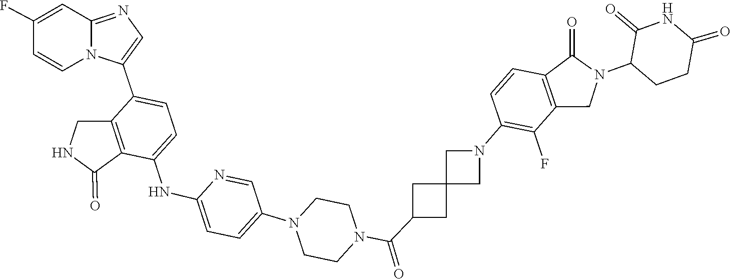 [SMILES: O=C1CCC(N2Cc3c(ccc(N4CC5(CC(C(=O)N6CCN(c7ccc(Nc8ccc(-c9cnc%10cc(F)ccn9%10)c9c8C(=O)NC9)nc7)CC6)C5)C4)c3F)C2=O)C(=O)N1] |
| 455 | 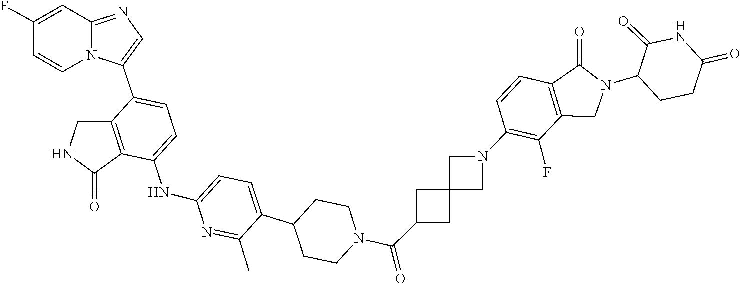 [SMILES: Cc1nc(Nc2ccc(-c3cnc4cc(F)ccn34)c3c2C(=O)NC3)ccc1C1CCN(C(=O)C2CC3(C2)CN(c2ccc4c(c2F)CN(C2CCC(=O)NC2=O)C4=O)C3)CC1] |
| 456 | 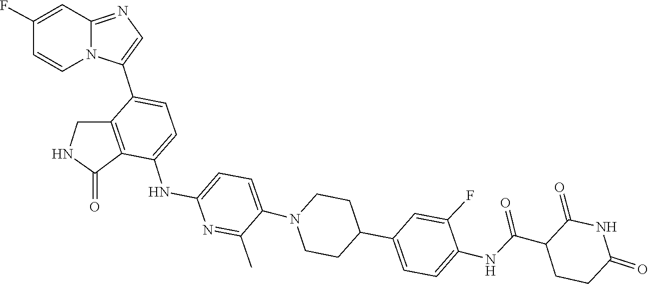 [SMILES: Cc1nc(Nc2ccc(-c3cnc4cc(F)ccn34)c3c2C(=O)NC3)ccc1N1CCC(c2ccc(NC(=O)C3CCC(=O)NC3=O)c(F)c2)CC1] |
| 457 | 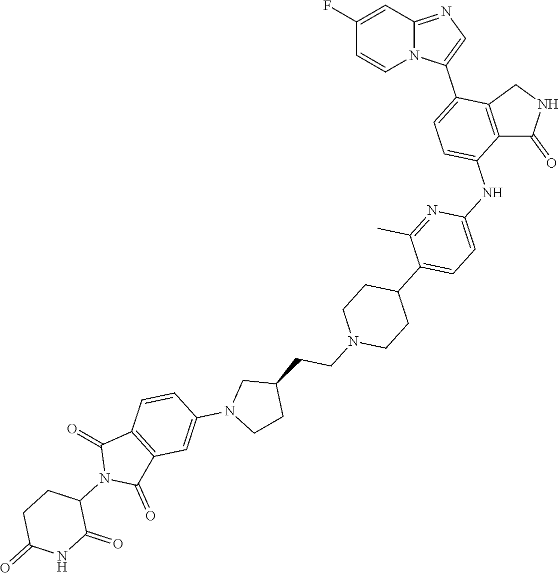 [SMILES: Cc1nc(Nc2ccc(-c3cnc4cc(F)ccn34)c3c2C(=O)NC3)ccc1C1CCN(CC[C@H]2CCN(c3ccc4c(c3)C(=O)N(C3CCC(=O)NC3=O)C4=O)C2)CC1] |

In another aspect, the compounds are described by the following numbered embodiments:

Embodiment 1. A compound of Formula (I):

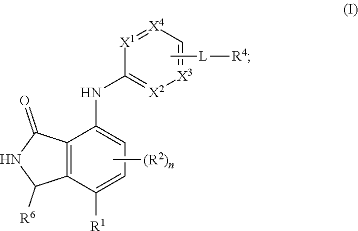

or a pharmaceutically acceptable salt thereof, wherein:
R¹ is C₃₋₆cycloalkyl, phenyl, 4- to 12-membered heterocyclyl or 5- to 10-membered heteroaryl, each of which is optionally substituted by one or more R⁵;
n is 0, 1, or 2;
each R² is independently halo, C₁₋₄alkyl, C₁₋₄haloalkyl, —OH, C₁₋₄alkoxy, C₁₋₄haloalkoxy, —N(C₁₋₄alkyl)₂, —NH₂, —NH(C₁₋₄alkyl), or cyano;
X¹, X², X³ and X⁴ are each independently N or —CR³, provided that at least two of X¹, X², X³ and X⁴ are —CR³.
each R³ is independently hydrogen, halo, —OR^{O3a}, —NR^{N3a}R^{N3b}, cyano, C₁₋₄alkyl, C₁₋₄haloalkyl, C₂alkynyl, —C(O)R^{3a}, —SO₂R^{3a}, —NR^{N3a}SO₂R^{3a}, C₃₋₆cycloalkyl, or 5- to 6-membered monocyclic heterocyclyl, wherein the C₁₋₄alkyl is optionally substituted by one or more R^{3a};
each R^{3a} is independently —OR^{O3a}, —NR^{N3a}R^{N3b}, or 5- to 6-membered monocyclic heterocyclyl;
R^{O3a}, R^{N3a}, and R^{N3b} are each independently hydrogen, C₁₋₄alkyl, or C₁₋₄haloalkyl;
R⁴ is:

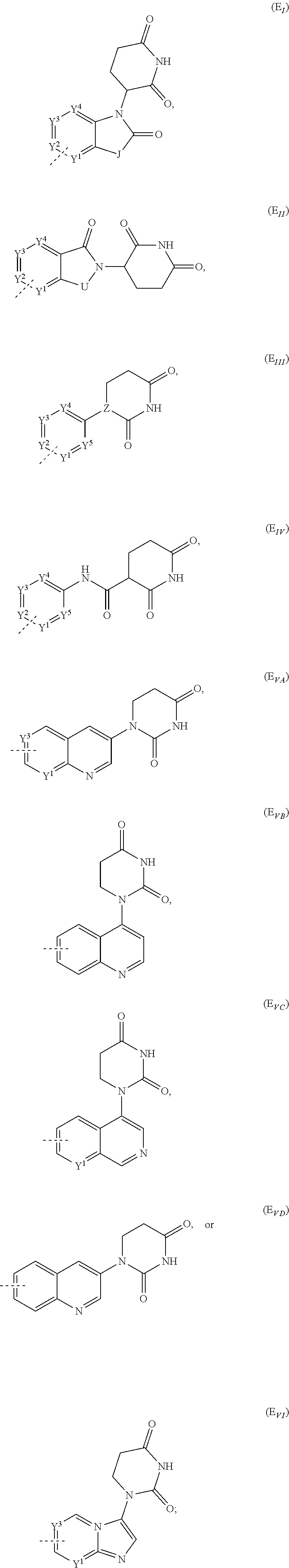

J is NR^{R1} or C(R^{E2})₂;
U is C═O or C(R^{E2})₂
R^{E1} is C₁₋₄alkyl or C₃₋₆cycloalkyl;
each R^{E2} is independently hydrogen, C₁₋₄alkyl, or C₁₋₄haloalkyl;
Z is N or CH;
each of Y¹, Y², Y³, Y⁴, and Y⁵ is independently CR^{Y} or N;
each R^{Y} is independently hydrogen, halo, C₁₋₃alkyl, C₁₋₃haloalkyl, C₁₋₃alkoxy, or C₁₋₃haloalkoxy
each R⁵ is independently halo, C₁₋₄alkyl, C₁₋₄haloalkyl, —OH, C₁₋₄alkoxy, C₁₋₄haloalkoxy, —N(C₁₋₄alkyl)₂, —NH₂, —NH(C₁₋₄alkyl), or cyano;
R⁶ is hydrogen, halo, C₁₋₄alkyl, or C₁₋₄haloalkyl; and
L is:

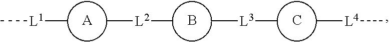

wherein:
Ring A, Ring B, and Ring C are each absent or a 4- to 10-membered carbocyclyl, 4- to 12-membered heterocyclyl, phenyl, or 5- to 10-membered heteroaryl, each of which are optionally substituted by one or more R^{L};
each R^{L} is independently halo, C₁₋₄alkyl, or C₁₋₄haloalkyl;
L¹, L², L³, and L⁴ are each absent or C₁₋₄alkylene, wherein the C₁₋₄alkylene is optionally substituted by one or more R^{L1}, and wherein one or more C in the C₁₋₄alkylene is optionally replaced by O; and
each R^{L1} is independently halo, or two R^{L1} groups, together with the carbon to which they are attached, form oxo or C₃₋₆cycloalkyl.
   Embodiment 2. The compound of embodiment 1, or a pharmaceutically acceptable salt thereof, wherein X⁴ and X³ are each CR³.
   Embodiment 3. The compound of embodiment 1 or 2, or a pharmaceutically acceptable salt thereof, wherein X¹ is N.
   Embodiment 4. The compound of any one of embodiments 1 to 3, or a pharmaceutically acceptable salt thereof, wherein X² is CR³.
   Embodiment 5. The compound of any one of embodiments 1 to 4, or a pharmaceutically acceptable salt thereof, wherein R¹ is a 9- to 12-membered bicyclic heterocyclyl or 8- to 10-membered bicyclic heteroaryl, each of which is optionally substituted by one or two R⁵.
   Embodiment 6. The compound of any one of embodiments 1 to 5, or a pharmaceutically acceptable salt thereof, wherein R¹ is a 9-membered bicyclic heterocyclyl or 9-membered bicyclic heteroaryl, each of which is optionally substituted by one or two R⁵.
   Embodiment 7. The compound of any one of embodiments 1 to 6, or a pharmaceutically acceptable salt thereof, wherein R¹ is 1H-pyrrolo[2,3-b]pyridinyl, 5,6,7,8-tetrahydroimidazo[1,2-a]pyridinyl, imidazo[1,2-a]pyrazinyl, imidazo[1,2-a]pyridinyl, or [1,2,4]triazolo[4,3-a]pyridinyl, each of which is optionally substituted by one or two R⁵.
   Embodiment 8. The compound of any one of embodiments 1 to 7, or a pharmaceutically acceptable salt thereof, wherein R¹ is:

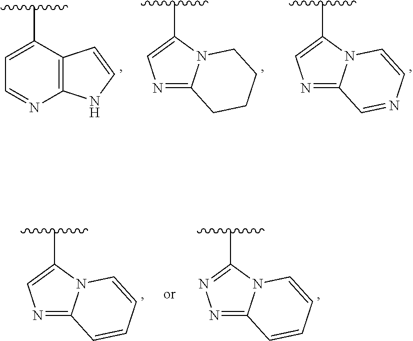

each of which is optionally substituted by one or two R⁵.
Embodiment 9. The compound of any one of embodiments 1 to 8, or a pharmaceutically acceptable salt thereof, wherein R¹ is:

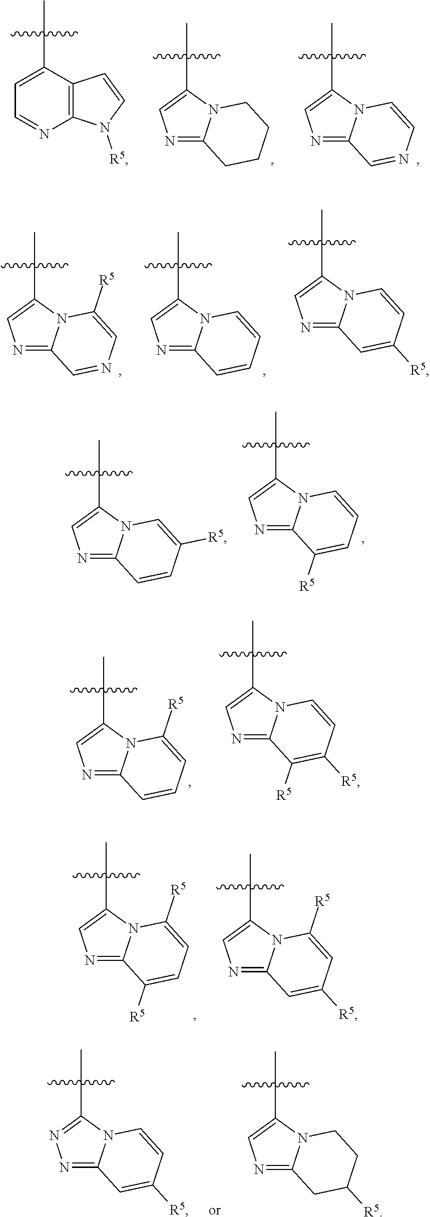

Embodiment 10. The compound of any one of embodiments 1 to 9, or a pharmaceutically acceptable salt thereof, wherein R¹ is:

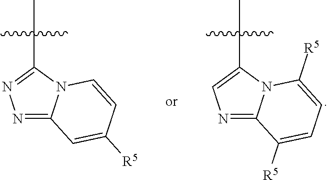

Embodiment 11. The compound of any one of embodiments 1 to 10, or a pharmaceutically acceptable salt thereof, wherein each R⁵ is independently halo, cyano, C₁₋₃alkoxy, C₁₋₃haloalkyl, or C₁₋₃alkyl.
Embodiment 12. The compound of any one of embodiments 1 to 11, or a pharmaceutically acceptable salt thereof, wherein each R⁵ is independently F, Cl, cyano, OCH₃, CF₃, or CH₃.
Embodiment 13. The compound of any one of embodiments 1 to 12, or a pharmaceutically acceptable salt thereof, wherein each R⁵ is independently F.
Embodiment 14. The compound of any one of embodiments 1 to 13, or a pharmaceutically acceptable salt thereof, wherein:
each R³ is independently halo, —OR^{O3a}, NR^{N3a}R^{N3b}, cyano, C₁₋₄alkyl, C₁₋₄haloalkyl, C₂alkynyl, or C₃₋₄cycloalkyl, wherein the C₁₋₄alkyl is optionally substituted by one or more R^{3a};
each R^{3a} is independently —OR^{O3a}, NR^{N3a}R^{N3b}, or 6-membered monocyclic heterocyclyl; and
R^{O3a}, R^{N3a}, and R^{N3b} are each independently hydrogen or C₁₋₄alkyl.
   Embodiment 15. The compound of any one of embodiments 1 to 14, or a pharmaceutically acceptable salt thereof, wherein each R³ is independently —F, —Cl, —CH₃, —CF₃, —CH₂CH₃, —CH₂CH₂CH₃, —CH(CH₃), —CH₂CH(CH₃)₂, —CH₂N(CH₃)₂, —CH₂OH, —CH₂F, —CHF₂, —CH(OH)CH₃, —C(CH₃)₂OH, —CH₂OCH₃, —C≡CH, —N(CH₃)₂, —OCH₃, cyano, cyclopropyl, or

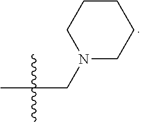 [SMILES: *CN1CCCCC1]

Embodiment 16. The compound of any one of embodiments 1 to 13, or a pharmaceutically acceptable salt thereof, wherein one of X², X³, and X⁴ is CR³, wherein R³ is C₁₋₃alkyl; and the remainder of X², X³, and X⁴ are CH.
Embodiment 17. The compound of any one of embodiments 1 to 13 or 16, or a pharmaceutically acceptable salt thereof, wherein each R³ is CH₃.
Embodiment 18. The compound of any one of embodiments 1 to 17, or a pharmaceutically acceptable salt thereof, wherein R⁶ is hydrogen or C₁₋₃alkyl.
Embodiment 19. The compound of any one of embodiments 1 to 18, or a pharmaceutically acceptable salt thereof, wherein R⁶ is hydrogen.
Embodiment 20. The compound of any one of embodiments 1 to 19, or a pharmaceutically acceptable salt thereof, wherein n is 0 or 1.
Embodiment 21. The compound of any one of embodiments 1 to 20, or a pharmaceutically acceptable salt thereof, wherein each R² is independently halo.
Embodiment 22. The compound of any one of embodiments 1 to 21, or a pharmaceutically acceptable salt thereof, wherein each R² is F.
Embodiment 23. The compound of any one of embodiments 1 to 20, or a pharmaceutically acceptable salt thereof, wherein n is 0.
Embodiment 24. The compound of any one of embodiments 1 to 17, wherein the compound is represented by Formula (II):

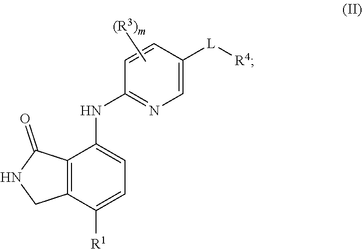

or a pharmaceutically acceptable salt thereof.
Embodiment 25. The compound of any one of embodiments 1 to 17, wherein the compound is represented by Formula (Ia), (Ib), (Ic), (Id), (Ie), (If), or (Ig):

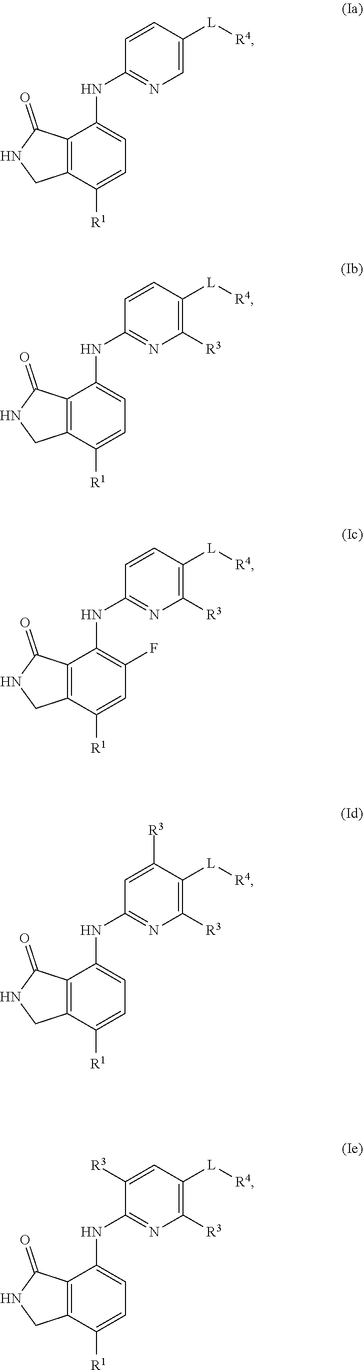

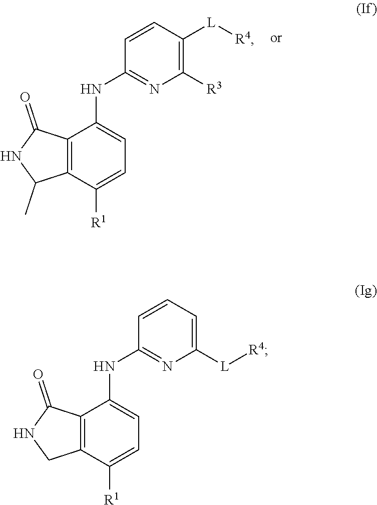

or a pharmaceutically acceptable salt thereof.
Embodiment 26. The compound of embodiment 1, wherein the compound is represented by Formula (III):

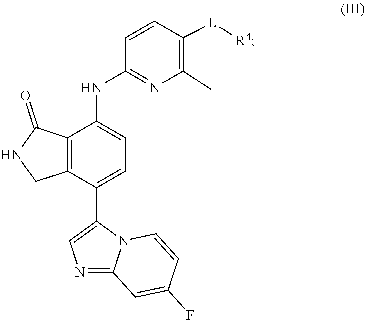

or a pharmaceutically acceptable salt thereof.
Embodiment 27. The compound of any one of embodiments 1 to 26, or a pharmaceutically acceptable salt thereof, wherein R⁴ is:

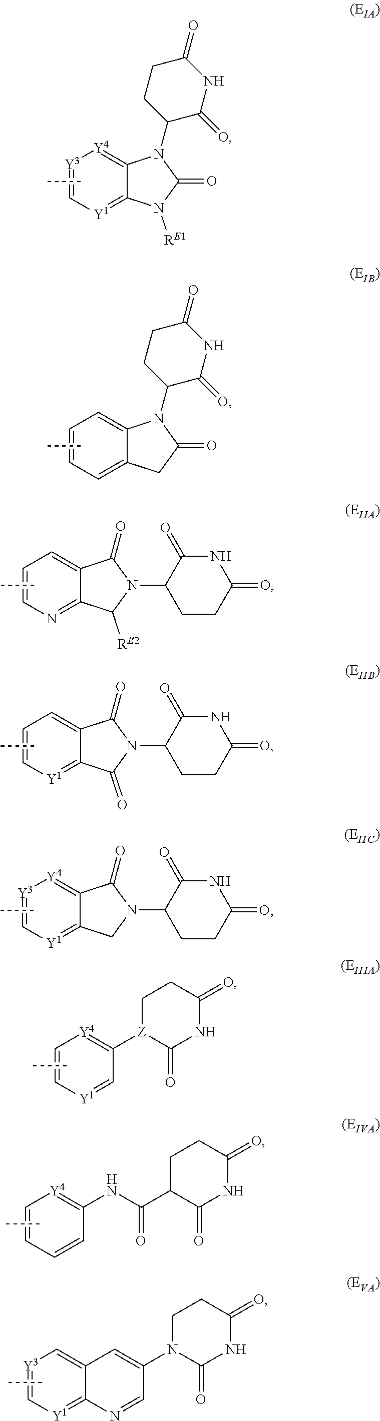

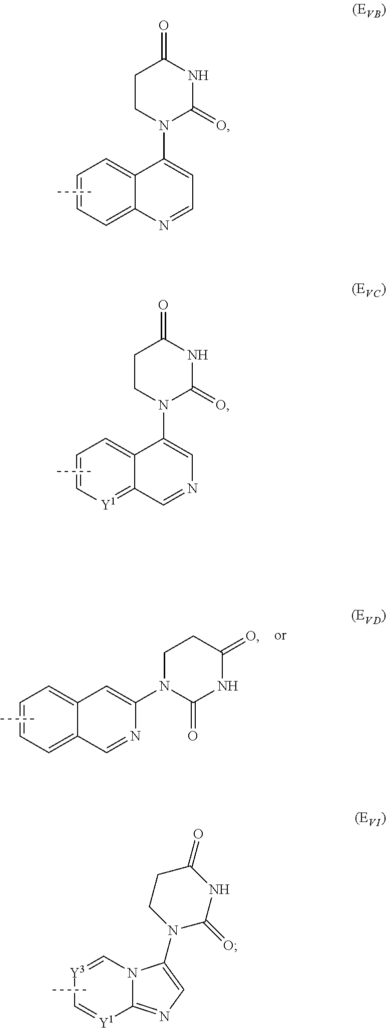

wherein:
Y¹, Y³, Y⁴ for E_{IA} CR^{Y};
Y¹ for and E_{IIB} is CR^{Y};
Y¹, Y³, and Y⁴ for E_{IIC} is CR^{Y};
Y¹ for E_{IIIA} is CR^{Y} or N, and Y⁴ for E_{IIIA} is CR^{Y};
Y⁴ for E_{IVA} is CR^{Y};
Y¹ and Y³ for E_{VA} is CR^{Y};
Y¹ for E_{VC} is CR^{Y};
Y¹ and Y³ for E_{VI} is CR^{Y}.
   Embodiment 28. The compound of any one of embodiments 1 to 27, or a pharmaceutically acceptable salt thereof, wherein R⁴ is:

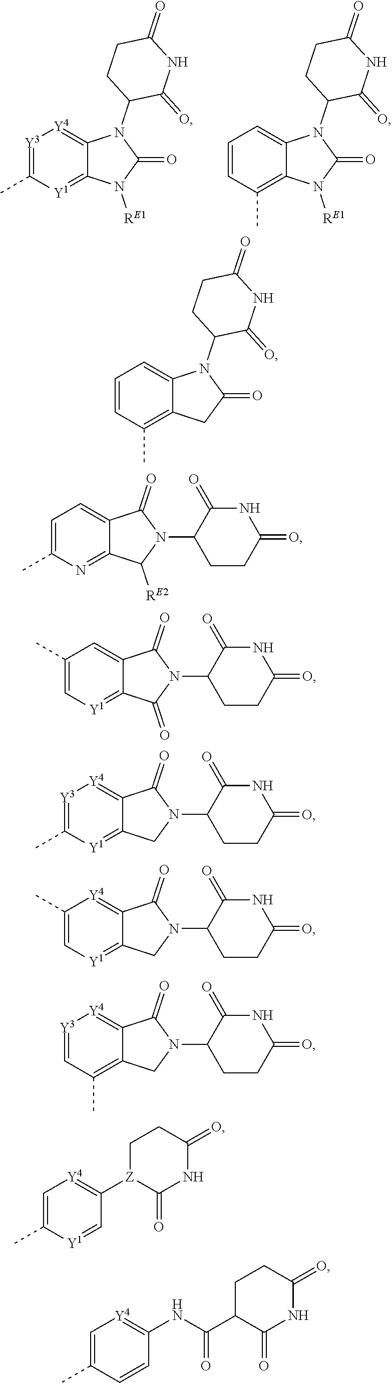

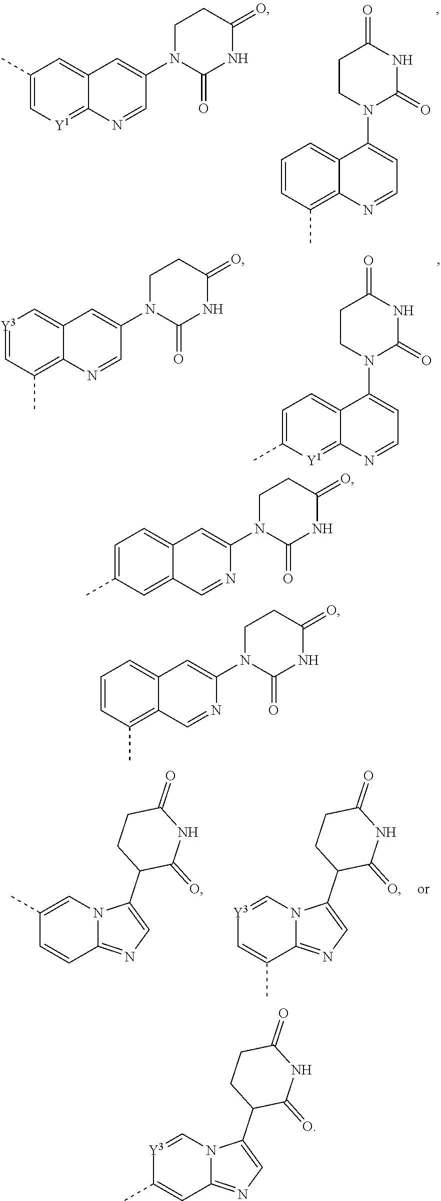

Embodiment 29. The compound of any one of embodiments 1 to 28, or a pharmaceutically acceptable salt thereof, wherein each R^{Y} is independently hydrogen, halo, C₁₋₃alkyl, or C₁₋₃alkoxy.
Embodiment 30. The compound of any one of embodiments 1 to 29, or a pharmaceutically acceptable salt thereof, wherein each R^{Y} is independently hydrogen, F, Cl, CH₃, or OCH₃.
Embodiment 31. The compound of any one of embodiments 1 to 30, or a pharmaceutically acceptable salt thereof, wherein R⁴ is:

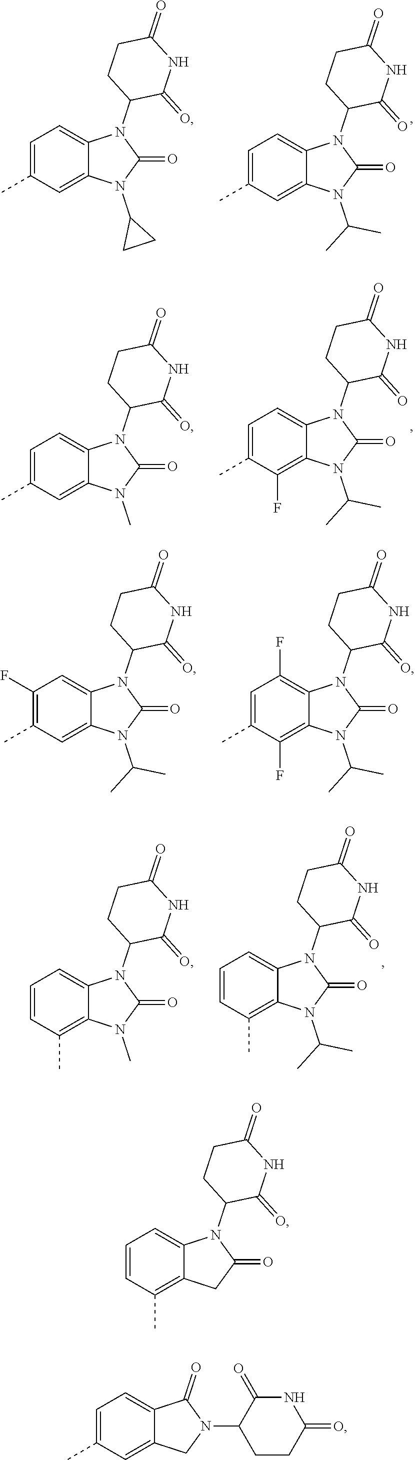

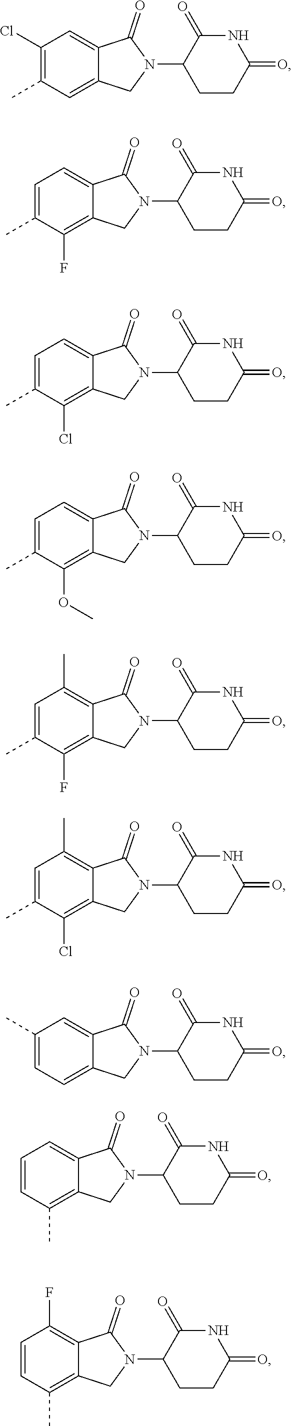

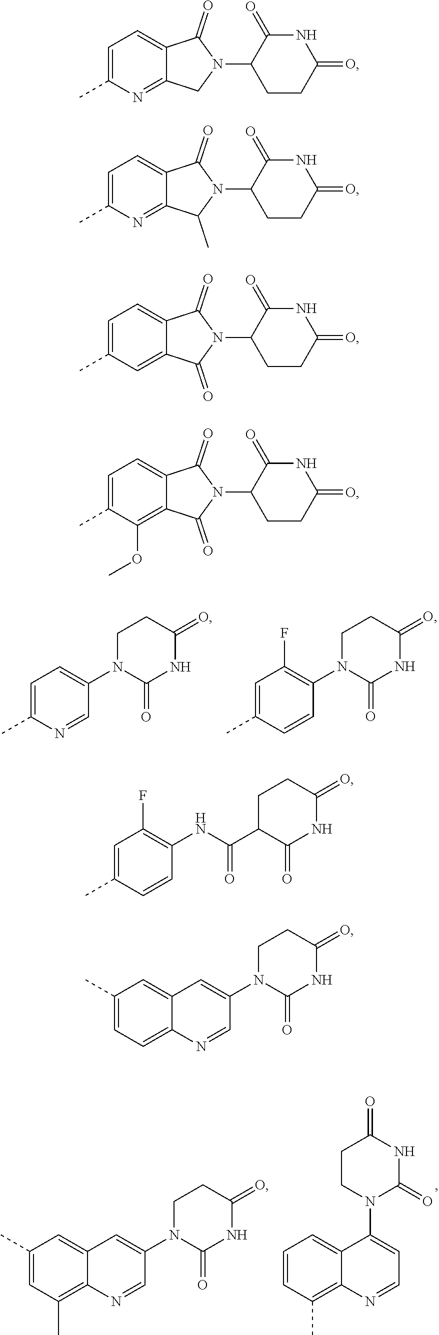

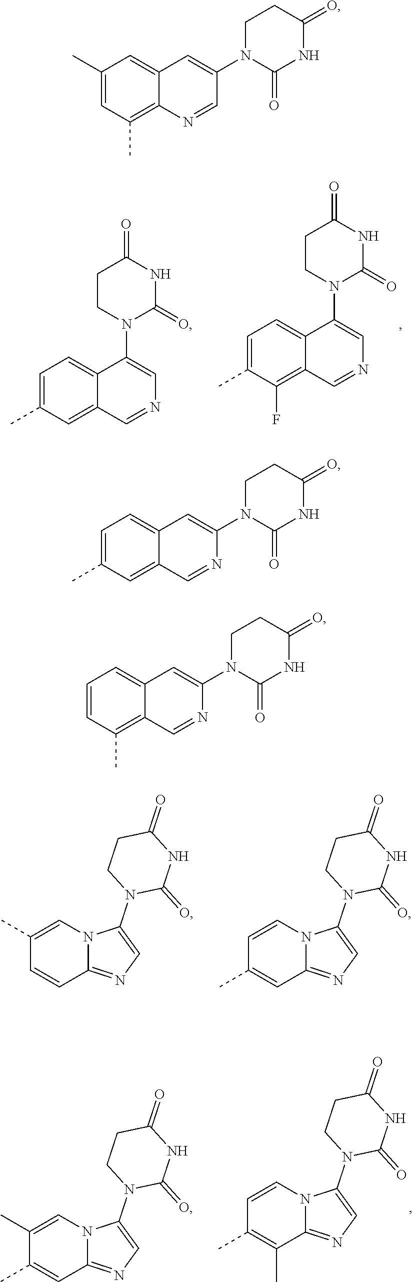

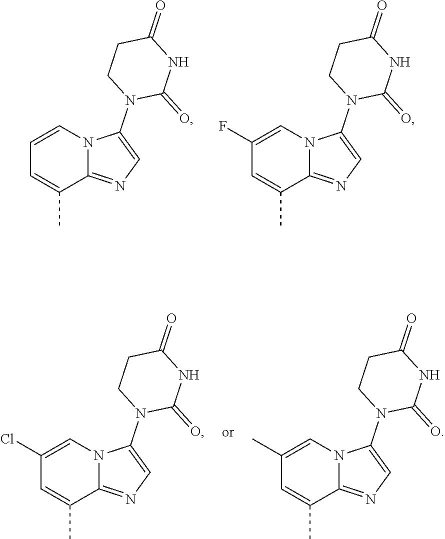

Embodiment 32. The compound of any one of embodiments 1 to 26, or a pharmaceutically acceptable salt thereof, wherein R⁴ is:

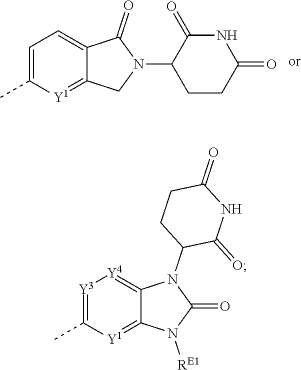

wherein:
R^{E1} S C₁₋₃alkyl or C₃₋₄cycloalkyl;
Y¹ is N or CR^{Y};
Y³ and Y⁴ are each independently CR^{Y};
each R^{Y} is independently hydrogen or halo.
   Embodiment 33. The compound of any one of embodiments 1 to 26, or 32, or a pharmaceutically acceptable salt thereof, wherein R⁴ is:

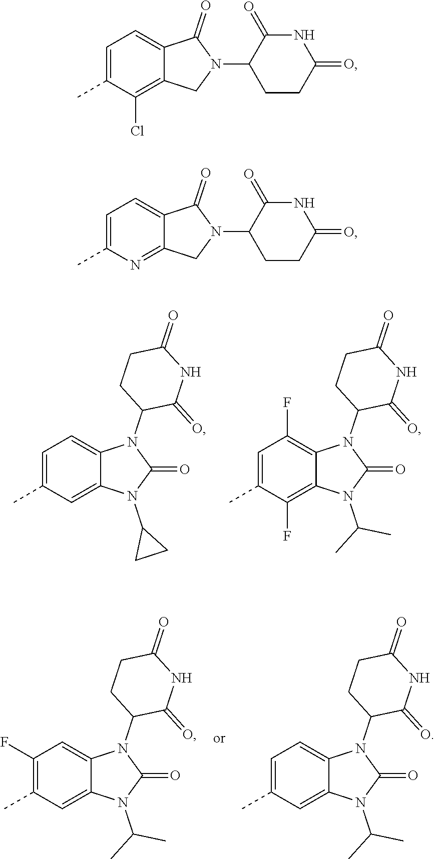

Embodiment 34. The compound of any one of embodiments 1 to 33, or a pharmaceutically acceptable salt thereof, wherein L¹ is absent, CH₂, or CH₂O.
Embodiment 35. The compound of any one of embodiments 1 to 34, or a pharmaceutically acceptable salt thereof, wherein L² is absent, CH₂, CH(CH)₃, C(O), or

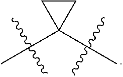 [SMILES: *C1(*)CC1]

Embodiment 36. The compound of any one of embodiments 1 to 35, or a pharmaceutically acceptable salt thereof, wherein L³ is absent or O.
Embodiment 37. The compound of any one of embodiments 1 to 36, or a pharmaceutically acceptable salt thereof, wherein L⁴ is absent.
Embodiment 38. The compound of any one of embodiments 1 to 37, or a pharmaceutically acceptable salt thereof, wherein Ring A is a 6-membered monocylic carbocyclyl, 6- to 10-membered heterocyclyl, or 5-membered monocyclic heteroaryl, each of which is optionally substituted by one or two R^{L}.
Embodiment 39. The compound of any one of embodiments 1 to 38, or a pharmaceutically acceptable salt thereof, wherein Ring A is cyclohexyl, cyclohexenyl, piperdinyl, piperazinyl, morpholinyl, 2,7-diazaspiro[3.5]nonanyl, 5-oxa-2,8-diazaspiro[3.5]nonanyl, 2-azabicyclo[2.2.1]heptanyl, 2,5-diazabicyclo[2.2.1]heptanyl, or pyrazolyl, each of which is optionally substituted by one or two R^{L}.
Embodiment 40. The compound of any one of embodiments 1 to 39, or a pharmaceutically acceptable salt thereof, wherein Ring B is absent, a 6-membered monocylic carbocyclyl, or a 4-to 12-membered heterocyclyl optionally substituted by one or two R^{L}.
Embodiment 41. The compound of any one of embodiments 1 to 40, or a pharmaceutically acceptable salt thereof, wherein Ring B is cyclohexyl, azetidinyl, pyrrolidinyl, piperdinyl, piperazinyl, 2-azaspiro[3.3]heptanyl, 3-azaspiro[5.5]undecanyl, 7-azaspiro[3.5]nonanyl, or 2-azaspiro[3.5]nonanyl, each of which is optionally substituted by one or two R^{L}.
Embodiment 42. The compound of any one of embodiments 1 to 41, or a pharmaceutically acceptable salt thereof, wherein Ring C is 6-membered monocyclic heterocyclyl.
Embodiment 43. The compound of any one of embodiments 1 to 42, or a pharmaceutically acceptable salt thereof, wherein Ring C is piperdinyl.
Embodiment 44. The compound of any one of embodiments 1 to 43, or a pharmaceutically acceptable salt thereof, wherein each R^{L} is independently halo or C₁₋₃alkyl.
Embodiment 45. The compound of any one of embodiments 1 to 44, or a pharmaceutically acceptable salt thereof, wherein each R^{L} is independently F or CH₃.
Embodiment 46. The compound of any one of embodiments 1 to 45, or a pharmaceutically acceptable salt thereof, wherein L is:

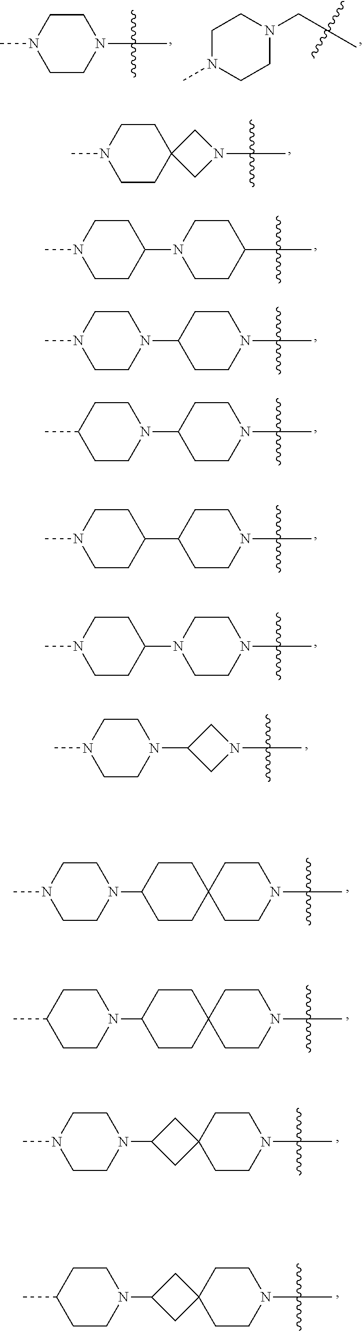

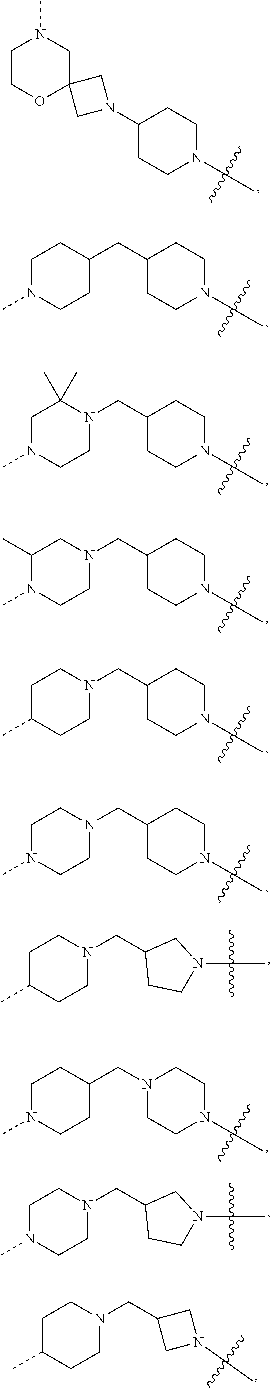

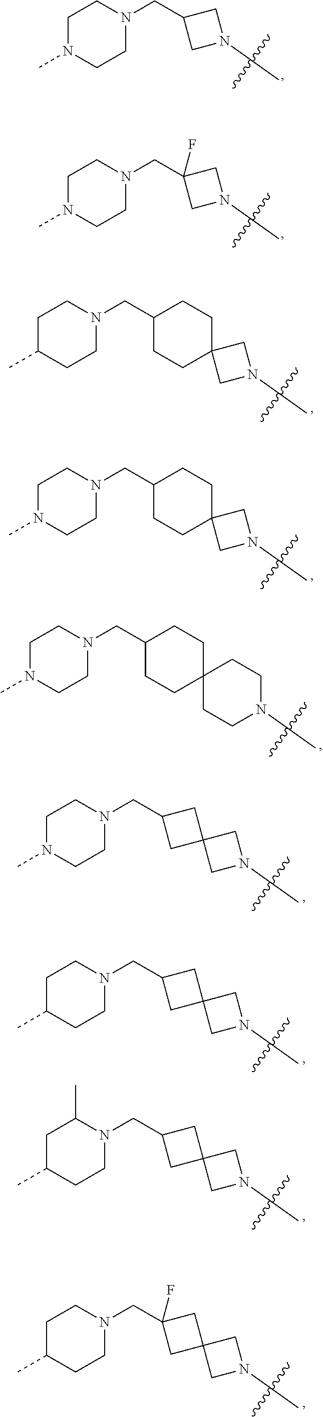

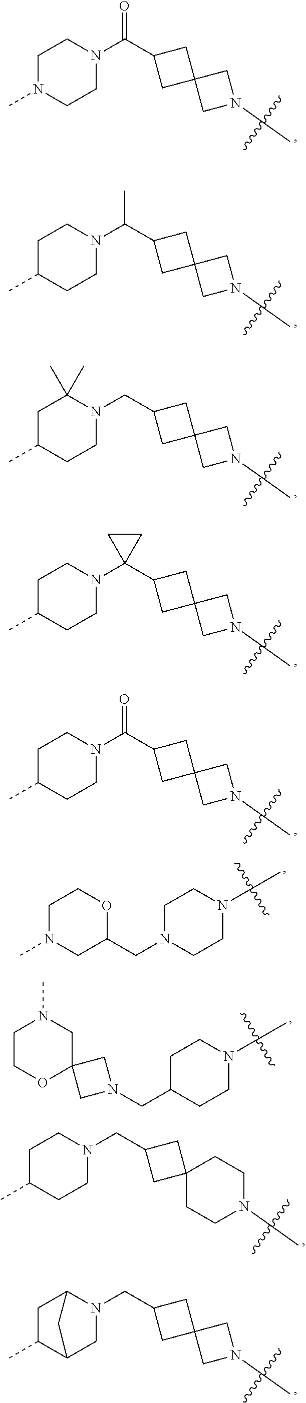

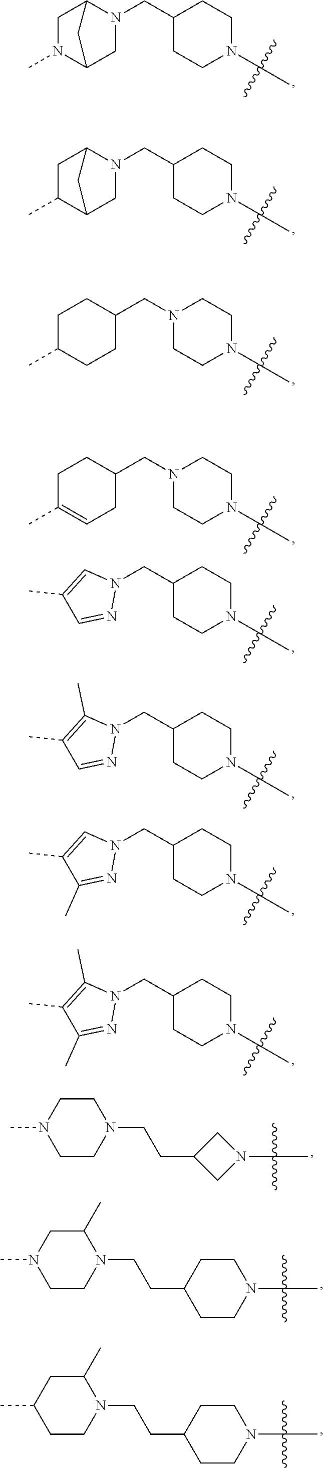

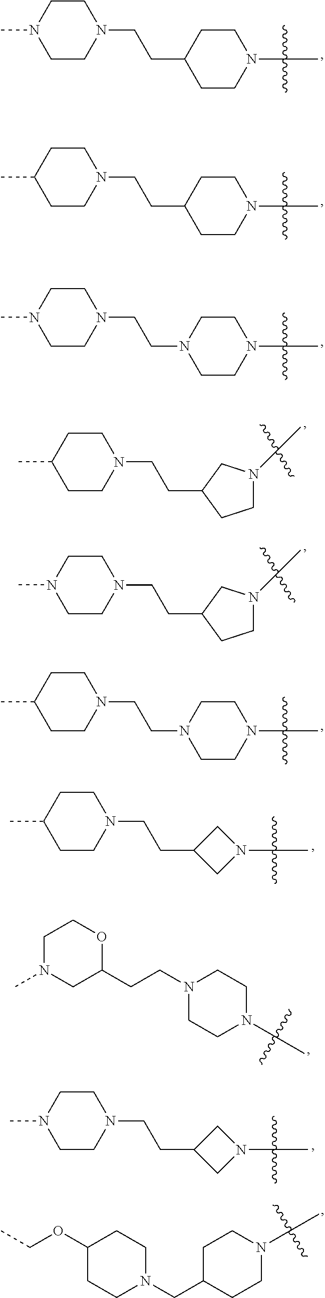

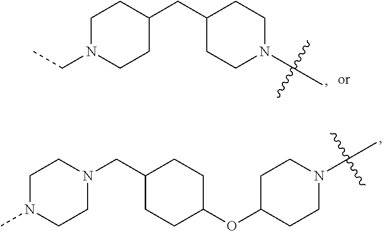

wherein

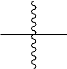

indicates the attachment point to R⁴.
Embodiment 47. The compound of any one of embodiments 1 to 33, or a pharmaceutically acceptable salt thereof, wherein L is:

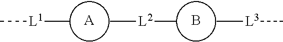

wherein:
Ring A and Ring B are each absent or a 4- to 10-membered carbocyclyl, 4- to 12-membered heterocyclyl, phenyl, or 5- to 10-membered heteroaryl, each of which are optionally substituted by one or more R^{L};
each R^{L} is independently halo, C₁₋₄alkyl, or C₁₋₄haloalkyl;
L¹, L², and L³ are each absent or C₁₋₄alkylene, wherein the C₁₋₄alkylene is optionally substituted by one or more R^{L1}, and wherein one or more C in the C₁₋₄alkylene is optionally replaced by O;
each R^{L1} is independently halo, or two R^{L1} groups, together with the carbon to which they are attached, form oxo or C₃₋₆cycloalkyl.
   Embodiment 48. The compound of any one of embodiments 1 to 33, or a pharmaceutically acceptable salt thereof, wherein L is:

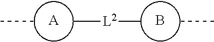

wherein Rings A and B are each independently 6-membered monocyclic heterocyclyl; and L² is absent or CH₂.
Embodiment 49. The compound of any one of embodiments 1 to 33 or 48, or a pharmaceutically acceptable salt thereof, wherein Ring A is piperdinyl or piperazinyl; and Ring B is piperdinyl.
Embodiment 50. The compound of any one of embodiments 1 to 33, 48, or 49, or a pharmaceutically acceptable salt thereof, wherein L is

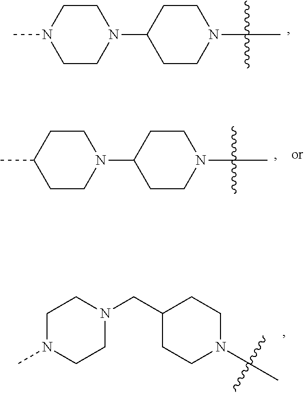

wherein

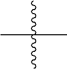

indicates the attachment point to R⁴.
Embodiment 51. The compound of embodiment 1, wherein the compound is represented by Formula (IV):

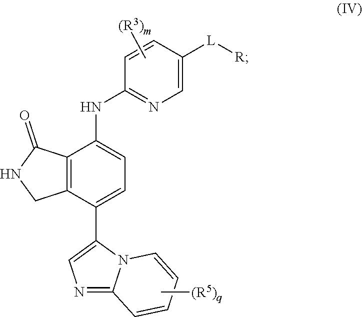

or a pharmaceutically acceptable salt thereof, wherein:
m is 0 or 1;
q is 0, 1, or 2;
each R³ and R⁵ is independently halo or C₁₋₂alkyl;

### L is

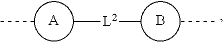

wherein:
Rings A and B are each independently 6-membered monocyclic heterocyclyl;
L² is absent or CH₂;

### R⁴ is

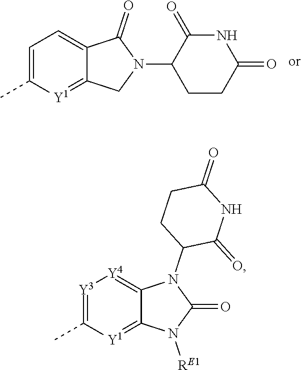

wherein
R^{E1} is C₁₋₃alkyl or C₃₋₄cycloalkyl;
Y¹ is N or CR^{Y};
Y³ and Y⁴ are each independently CR^{Y};
each R^{Y} is independently hydrogen or halo.
   Embodiment 52. The compound of embodiment 51, or a pharmaceutically acceptable salt thereof wherein:
m is 1;
q is 1 or 2;
R³ is CH₃;
each R⁵ is F.
   Embodiment 53. The compound of embodiment 51 or 52, wherein the compound is represented by Formula (III):

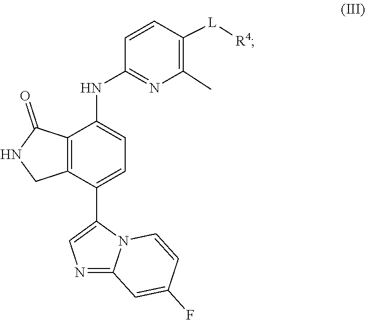

or a pharmaceutically acceptable salt thereof.
Embodiment 54. The compound of any one of embodiments 51 to 53, or a pharmaceutically acceptable salt thereof, wherein Rings A and B are each independently piperazinyl or piperidinyl.
Embodiment 55. The compound of any one of embodiments 51 to 54, or a pharmaceutically acceptable salt thereof, wherein Ring A is piperazinyl and Ring B is piperdinyl.
Embodiment 56. The compound of any one of embodiments 51 to 55, or a pharmaceutically acceptable salt thereof, wherein L² is —CH₂—.
Embodiment 57. The compound of any one of embodiments 51 to 56, or a pharmaceutically acceptable salt thereof, wherein R⁴ is

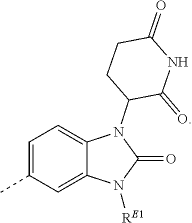

Embodiment 58. The compound of any one of embodiments 51 to 57, or a pharmaceutically acceptable salt thereof, wherein R^{E1} is —CH(CH₃)₂.

Compounds of Formula (I) are further described in the Exemplification and are included in the present disclosure. Pharmaceutically acceptable salts thereof as well as the neutral forms of the compounds described herein are also included.

Also provided herein are pharmaceutical compositions comprising a described compound or a pharmaceutically acceptable salt of a described compound, or pharmaceutical compositions comprising a described compound or a pharmaceutically acceptable salt of a compound described herein; and a pharmaceutically acceptable carrier.

### 4. Uses and Administration

The compounds and compositions described herein are generally useful for treating a HPK1 related condition. Thus, in one aspect, provided are methods of treating a HPK1 related condition in a subject in need thereof comprising administering to the subject a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a HPK1 related condition. Also provided is a compound described herein, or a pharmaceutically acceptable salt thereof, or a pharmaceutical composition comprising a disclosed compound or pharmaceutically acceptable salt thereof, for use in treating a HPK1 related condition.

In one aspect, the HPK1 related conditions are those which are modulated by degrading the HPK1 protein.

In certain aspects, the HPK1 related condition is cancer. In certain aspects the cancer is a solid tumor. In certain aspect the cancer is selected from melanoma, lung cancer, head and neck cancer, prostate cancer, colorectal cancer, or renal cancer. In certain aspects, the cancer is non-small cell lung cancer, Squamous cell carcinoma of the head and neck, metastatic castration resistant prostate cancer, or MSS colorectal cancer.

In certain aspect the cancer is selected from melanoma, lung cancer, head and neck cancer, prostate cancer, colorectal cancer, or renal cancer. In certain aspects, the cancer is non-small cell lung cancer, Squamous cell carcinoma of the head and neck, metastatic castration resistant prostate cancer, MSS colorectal cancer, renal cell carcinoma, bladder cancer, small cell lung carcinoma, or gastric cancer.

The compounds, the pharmaceutically acceptable salts of the compounds, and the pharmaceutical compositions described herein may be administered orally, parenterally, by inhalation spray, topically, rectally, nasally, buccally, vaginally or via an implanted reservoir. Preparations for such pharmaceutical compositions are well-known in the art. See, e.g., Anderson, Philip O.; Knoben, James E.; Troutman, William G., eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety. The term “parenteral” as used herein includes subcutaneous, intravenous, intramuscular, intra-articular, intra-synovial, intrasternal, intrathecal, intrahepatic, intralesional and intracranial injection or infusion techniques. Liquid dosage forms, injectable preparations, solid dispersion forms, and dosage forms for topical or transdermal administration of the compounds, the pharmaceutically acceptable salts of the compounds, and the pharmaceutical compositions described herein are included herein. In one aspect, the compounds, the pharmaceutically acceptable salts of the compounds, and the pharmaceutical compositions described herein are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### ABBREVIATIONS

ACN Acetonitrile
CDCl₃ Chloroform-d
DCM Dichloromethane
FA Formic acid
DMSO Dimethyl sulfoxide
DMSO-d₆ Deuterated dimethyl sulfoxide (C₂D₆SO)
ESI Electrospray ionization
EtOH Ethanol
eq Equivalent(s)
g Gram(s)
h or hr. Hour(s)
Hz Hertz
HCl Hydrochloric acid
HPLC High performance liquid chromatography
LCMS Liquid chromatography-mass spectrometry
m/z Mass/charge
MS Mass spectrometry
MHz Megahertz
mm Millimeter(s)
mL Milliliter(s)
mM Millimolar
mmol Millimole(s)
NaBH(OAc)₃ Sodium triacetoxyborohydride
TLC Thin-layer chromatography
Xphos 2-Dicyclohexylphosphino-2′,4′,6′-triisopropylbiphenyl
Xphos Pd G2 Chloro(2-dicyclohexylphosphino-2′,4′,6′-triisopropyl-1,1′-biphenyl)[2-(2′-amino-1,1′-biphenyl)]palladium(II)
Xphos Pd G3 (2-Dicyclohexylphosphino-2′,4′,6′-triisopropyl-1,1′-biphenyl)[2-(2′-amino-1,1′-biphenyl)]palladium(II) methanesulfonate

### EXEMPLIFICATION

### General Methods

### Exemplification of Method A

### Example 1: Exemplary Synthesis of 3-[5-[4-[[4-[6-[[7-(7-fluoroimidazo[1,2-a]pyridin-3-yl)-3-oxo-isoindolin-4-yl]amino]-2-methyl-3-pyridyl]piperazin-1-yl]methyl]-1-piperidyl]-3-isopropyl-2-oxo-benzimidazol-1-yl]piperidine-2,6-dione

### Step 1: Preparation of tert-butyl 4-(2-methyl-3-pyridyl)piperazine-1-carboxylate

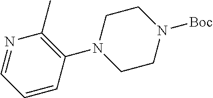 [SMILES: Cc1ncccc1N1CCN(C(=O)OC(C)(C)C)CC1]

A mixture of 3-bromo-2-methyl-pyridine (13.5 g, 78 mmol), tert-butyl piperazine-1-carboxylate (17.5 g, 94 mmol), cesium carbonate (51.1 g, 157 mmol) and Ruphos Pd G3 (3.3 g, 4 mmol) in dioxane (150 mL) was degassed and purged with nitrogen several times, and the mixture was stirred at 100° C. for 12 h under nitrogen atmosphere. The reaction mixture was filtered and the filtrate was concentrated under reduced pressure. The crude product was purified by column chromatography on silica gel (petroleum ether/ethyl acetate=1:0 to 3:1) to afford the title compound (16 g, 73%) as a yellow oil. MS (ESI) m/z: 278.1 [M+H]⁺; ¹H NMR (400 MHz, DMSO-d₆) δ 8.24 (dd, J=1.6, 4.8 Hz, 1H), 7.28-7.24 (m, 1H), 7.12 (dd, J=4.8, 8.0 Hz, 1H), 3.64-3.52 (m, 4H), 2.92-2.80 (m, 4H), 2.56 (s, 3H), 1.48 (s, 9H).

### Step 2: Preparation of tert-butyl 4-(6-bromo-2-methyl-3-pyridyl)piperazine-1-carboxylate

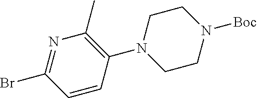 [SMILES: Cc1nc(Br)ccc1N1CCN(C(=O)OC(C)(C)C)CC1]

To a solution of tert-butyl 4-(2-methyl-3-pyridyl)piperazine-1-carboxylate (16 g, 58 mmol) in acetonitrile (200 mL) was added 1-bromopyrrolidine-2,5-dione (10.3 g, 58 mmol) at 0° C. The mixture was stirred at 0° C. for 1 h, then concentrated under reduced pressure. The residue was purified by column chromatography (petroleum ether/ethyl acetate=1:0 to 5:1) to afford the title compound (5.8 g, 28%) as a white solid. MS (ESI) m/z: 356.1 [M+H]⁺; ¹H NMR (400 MHz, DMSO-d₆) δ 7.44-7.28 (m, 2H), 3.48 (s, 4H), 2.84-2.76 (m, 4H), 2.40 (s, 3H), 1.44 (s, 9H).

### Step 3: Preparation of 4-bromo-7-nitro-isoindolin-1-one

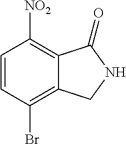 [SMILES: O=C1NCc2c(Br)ccc([N+](=O)[O-])c21]

To a solution of 4-bromoisoindolin-1-one (50 g, 0.2 mol) in sulfuric acid (400 mL, 7.5 mol) was added nitric acid (31 mL, 0.5 mol, 68%) at −10° C. The mixture was stirred at 25° C. for 4 h, then poured into ice water (100 mL). The suspension was filtered, the filter cake was washed with water (200 mL), then dried to afford the title compound (53 g, 87%) as a white solid. MS (ESI) m/z: 258.8 [M+H]⁺; ¹H NMR (400 MHz, DMSO-d₆) δ 9.19 (s, 1H), 8.05 (d, J=8.4 Hz, 1H), 7.87 (d, J=8.4 Hz, 1H), 4.39 (s, 2H).

### Step 4: Preparation of tert-butyl 4-bromo-7-nitro-1-oxo-isoindoline-2-carboxylate

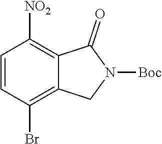 [SMILES: CC(C)(C)OC(=O)N1Cc2c(Br)ccc([N+](=O)[O-])c2C1=O]

To a solution of 4-bromo-7-nitro-isoindolin-1-one (23 g, 89 mmol) in dichloromethane (200 mL) was added trimethylamine (37.0 mL, 268 mmol), dimethylaminopyridine (1.09 g, 9 mmol) and di-tert-butyl dicarbonate (23.43 g, 107 mmol). The mixture was stirred at 25° C. for 2 h. The reaction mixture was diluted with water (800 mL) and extracted with ethyl acetate (150 mL×3). The combined organic phase was washed with saturated brine (800 mL), dried over anhydrous sodium sulfate, filtered, and concentrated under reduced pressure. The residue was purified by flash silica gel chromatography (15-100% ethyl acetate/petroleum ether) to afford the title compound (29.3 g, 92%) as a yellow solid. MS (ESI) m/z: 300.9 [M−56+H]⁺; ¹H NMR (400 MHz, DMSO-d₆) δ 8.18 (d, J=8.4 Hz, 1H), 7.95 (d, J=8.4 Hz, 1H), 4.73 (s, 2H), 1.52 (s, 9H).

### Step 5: Preparation of tert-butyl 7-amino-4-bromo-1-oxo-isoindoline-2-carboxylate

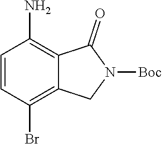 [SMILES: CC(C)(C)OC(=O)N1Cc2c(Br)ccc(N)c2C1=O]

To a solution of tert-butyl 4-bromo-7-nitro-1-oxo-isoindoline-2-carboxylate (24 g, 67 mmol) in ethanol (200 mL) and water (40 mL) were slowly added iron powder (18.76 g, 336 mmol) and ammonium chloride (35.94 g, 672 mmol). The mixture was stirred at 60° C. for 2 h, then filtered, and concentrated. The residue was purified by flash silica gel chromatography (0-30% ethyl acetate/petroleum ether) to afford the title compound (10 g, 45%) as a yellow solid. MS (ESI) m/z: 227.1 [M−100+H]⁺; ¹H NMR (400 MHz, DMSO-d₆) δ 7.42 (d, J=8.8 Hz, 1H), 6.61 (d, J=8.8 Hz, 1H), 6.57-6.32 (m, 2H), 4.50 (s, 2H), 1.51 (s, 9H).

### Step 6: Preparation of tert-butyl 7-amino-4-(7-fluoroimidazo[1,2-a]pyridin-3-yl)-1-oxo-isoindoline-2-carboxylate

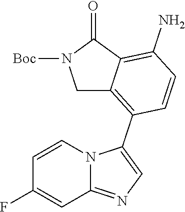 [SMILES: CC(C)(C)OC(=O)N1Cc2c(-c3cnc4cc(F)ccn34)ccc(N)c2C1=O]

A mixture of tert-butyl 7-amino-4-bromo-1-oxo-isoindoline-2-carboxylate (10 g, 30 mmol), 7-fluoroimidazo[1,2-a]pyridine (6.24 g, 46 mmol), palladium(II) acetate (686 mg, 3 mmol) and potassium acetate (6.00 g, 61 mmol) in N,N-dimethylacetamide (100 mL) was degassed and purged with nitrogen several times, the mixture was stirred at 110° C. for 12 h under nitrogen atmosphere. The reaction mixture was diluted with water (800 mL) and extracted with ethyl acetate (1200 mL×3). The combined organic phase was washed with saturated brine (600 mL), dried over anhydrous sodium sulfate, filtered, and concentrated under reduced pressure. The residue was purified by flash silica gel chromatography (40-100% ethyl acetate/petroleum ether) to afford the title compound (8.8 g, 75%) as a yellow solid. MS (ESI) m/z: 383.3 [M+H]⁺; ¹H NMR (400 MHz, DMSO-d₆) δ 8.28-8.20 (m, 1H), 7.71 (s, 1H), 7.52-7.45 (m, 2H), 6.95 (dt, J=2.4, 7.6 Hz, 1H), 6.80 (d, J=8.4 Hz, 1H), 6.67 (s, 2H), 4.59 (s, 2H), 1.47 (s, 9H).

### Step 7: Preparation of tert-butyl 7-[[5-(4-tert-butoxycarbonylpiperazin-1-yl)-6-methyl-2-pyridyl]amino]-4-(7-fluoroimidazo[1,2-a]pyridin-3-yl)-1-oxo-isoindoline-2-carboxylate

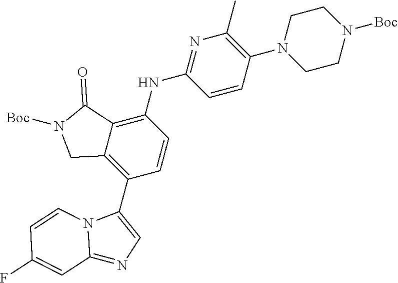 [SMILES: Cc1nc(Nc2ccc(-c3cnc4cc(F)ccn34)c3c2C(=O)N(C(=O)OC(C)(C)C)C3)ccc1N1CCN(C(=O)OC(C)(C)C)CC1]

To a mixture of tert-butyl 4-(6-bromo-2-methyl-3-pyridyl)piperazine-1-carboxylate (1 g, 3 mmol) and tert-butyl 7-amino-4-(7-fluoroimidazo[1,2-a]pyridin-3-yl)-1-oxo-isoindoline-2-carboxylate (966 mg, 2.5 mmol) in dioxane (20 mL) were added (1E,4E)-1,5-diphenylpenta-1,4-dien-3-one; palladium (257 mg, 0.3 mmol), (5-diphenylphosphanyl-9,9-dimethyl-xanthen-4-yl)-diphenyl-phosphane (162 mg, 0.3 mmol) and tripotassium phosphate (1.8 g, 8 mmol) under nitrogen. The mixture was stirred at 100° C. for 12 h, then filtered, and concentrated under reduced pressure. The residue was purified by column chromatography (petroleum ether/ethyl acetate=1:0 to 1:1) to afford the title compound (550 mg, 29%) as a yellow solid. MS (ESI) m/z: 658.4 [M+H]⁺.

### Step 8: Preparation of 4-(7-fluoroimidazo[1,2-a]pyridin-3-yl)-7-[(6-methyl-5-piperazin-1-yl-2-pyridyl)amino]isoindolin-1-one

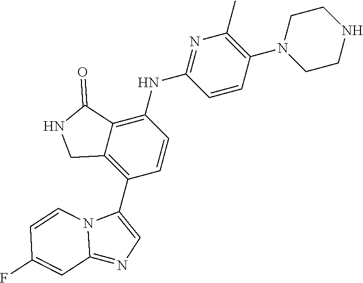 [SMILES: Cc1nc(Nc2ccc(-c3cnc4cc(F)ccn34)c3c2C(=O)NC3)ccc1N1CCNCC1]

To a mixture of tert-butyl 7-[[5-(4-tert-butoxycarbonylpiperazin-1-yl)-6-methyl-2-pyridyl]amino]-4-(7-fluoroimidazo[1,2-a]pyridin-3-yl)-1-oxo-isoindoline-2-carboxylate (550 mg, 0.8 mmol) in dichloromethane (3 mL) was added trifluoroacetic acid (1 mL). The mixture was stirred at 25° C. for 0.5 h, then concentrated under reduced pressure to afford the title compound (570 mg, crude, 2 trifluoroacetate) as a brown oil. MS (ESI) m/z: 458.2 [M+H]⁺.

### Step 9: Preparation of 3-[4-(dimethoxymethyl)-1-piperidyl]-2-nitro-aniline

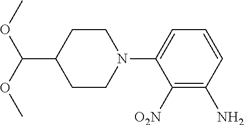 [SMILES: COC(OC)C1CCN(c2cccc(N)c2[N+](=O)[O-])CC1]

To a solution of 3-fluoro-2-nitro-aniline (2.0 g, 13 mmol) and 4-(dimethoxymethyl)piperidine (2.04 g, 13 mmol) in N,N-dimethylformamide (20 mL) was added cesium carbonate (8.35 g, 26 mmol). The reaction mixture was stirred at 100° C. for 12 h, then cooled to 25° C. The mixture was diluted with water (60 mL) and extracted with ethyl acetate (30 mL×3). The combined organic layer was washed with brine (30 mL×3). The organic layer was dried over anhydrous sodium sulfate, filtered, and concentrated under reduced pressure. The residue was purified by silica gel column chromatography (petroleum ether/ethyl acetate=10/1 to 4:1) to afford the title compound (3 g, 79%) as a red solid. MS (ESI) m/z: 296.0 [M+H]⁺.

### Step 10: Preparation of 2,6-bis(benzyloxy)-3-nitropyridine

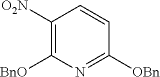 [SMILES: O=[N+]([O-])c1ccc(OCc2ccccc2)nc1OCc1ccccc1]

To a mixture of 2,6-dichloro-3-nitropyridine (25.0 g, 156 mmol) and phenylmethanol (40.6 mL, 390 mmol) in acetonitrile (500 mL) was added cesium carbonate (127.2 g, 390 mmol). The mixture was stirred at 60° C. for 6 h, then filtered, and concentrated under reduced pressure. The crude product was triturated with petroleum ether/2-methoxy-2-methylpropane (1:1, 500 mL) to afford the title compound (49 g, 93%) as yellow solid.

### Step 11: Preparation of 2,6-bis(benzyloxy)pyridin-3-amine

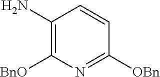 [SMILES: Nc1ccc(OCc2ccccc2)nc1OCc1ccccc1]

To a mixture of 2,6-bis(benzyloxy)-3-nitropyridine (95.0 g, 282 mmol) and ammonium chloride (226.6 g, 4.2 mol) in isopropanol (950 mL) and water (475 mL) was added iron (126.2 g, 2.3 mol) under nitrogen. The mixture was heated to 90° C. and stirred for 16 h. The reaction mixture was cooled to 20° C. and filtered, the filtrate solution was poured into ice-water (w/w=1/1, 1000 mL) and stirred for 5 min. The resulting mixture was extracted with ethyl acetate (1000 mL×2). The combined organic phase was washed with brine (1000 mL×3), dried over anhydrous sodium sulfate, filtered, and concentrated under reduced pressure. The residue was purified by prep-HPLC (1%-20% ethanol in n-hexane over 15 min) to afford the title compound (61 g, 70%) as brown oil. MS (ESI) m/z: 307.2 [M+H]⁺.

### Step 12: Preparation of 2,6-dibenzyloxy-N-(4-bromo-2-nitro-phenyl)pyridin-3-amine

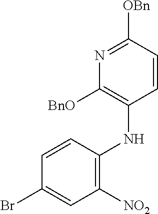 [SMILES: O=[N+]([O-])c1cc(Br)ccc1Nc1ccc(OCc2ccccc2)nc1OCc1ccccc1]

To a solution of 2,6-dibenzyloxypyridin-3-amine (5.0 g, 16 mmol) and 4-bromo-1-fluoro-2-nitro-benzene (2.4 mL, 20 mmol) in tetrahydrofuran (50 mL) was added lithium bis(trimethylsilyl)amide (1 M in tetrahydrofuran, 24.5 mL). The mixture was stirred at −78° C. for 1 h, then warmed to 25° C. and stirred for 11 h under nitrogen atmosphere. The mixture was diluted with saturated ammonium chloride solution until pH=8-9, the resulting mixture was extracted with ethyl acetate (300 mL). The combined organic layers were washed with brine (20 mL×3), dried over anhydrous sodium sulfate, filtered and concentrated. The residue was purified by silica gel column chromatography (petroleum ether/ethyl acetate=1/0 to 100/1) to afford the title compound (9 g, crude) as a brown oil. MS (ESI) m/z: 508.1 [M+H]⁺.

### Step 13: Preparation of 4-bromo-N1-(2,6-dibenzyloxy-3-pyridyl)benzene-1,2-diamine

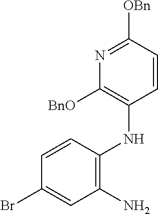 [SMILES: Nc1cc(Br)ccc1Nc1ccc(OCc2ccccc2)nc1OCc1ccccc1]

To a solution of 2,6-dibenzyloxy-N-(4-bromo-2-nitro-phenyl)pyridin-3-amine (8.0 g, 16 mmol) in ethanol (100 mL) and water (50 mL) was added iron (4.41 g, 79 mmol) and saturated aqueous ammonium chloride solution (8.45 g, 0.15 mol). The mixture was stirred at 80° C. for 4 h, then filtered. The filtrate was extracted with ethyl acetate (300 mL). The organic layer was washed with brine (30 mL×3), dried over anhydrous sodium sulfate, filtered and concentrated. The residue was purified by silica gel column chromatography (petroleum ether/ethyl acetate=1/0 to 10/1) to afford the title compound (2.8 g, 37%) as a brown solid.

### Step 14: Preparation of 6-bromo-3-(2,6-dibenzyloxy-3-pyridyl)-1H-benzimidazol-2-one

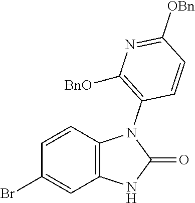 [SMILES: O=c1[nH]c2cc(Br)ccc2n1-c1ccc(OCc2ccccc2)nc1OCc1ccccc1]

A mixture of 4-bromo-N1-(2,6-dibenzyloxy-3-pyridyl)benzene-1,2-diamine (2.8 g, 6 mmol) and 4-dimethylaminopyridine (4.77 g, 29 mmol) in N,N-dimethyl formamide (30 mL) was stirred at 120° C. for 2 h. The reaction mixture was suspended in water and filtered. The filter cake was dried under vacuum to afford the title compound (2.8 g, crude) as a yellow solid, which was used in the next step directly. MS (ESI) m/z: 504.1 [M+H]⁺.

### Step 15: Preparation of 5-bromo-1-(2,6-dibenzyloxy-3-pyridyl)-3-isopropyl-benzimidazol-2-one

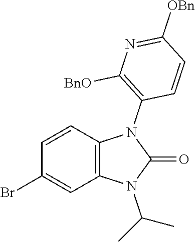 [SMILES: CC(C)n1c(=O)n(-c2ccc(OCc3ccccc3)nc2OCc2ccccc2)c2ccc(Br)cc21]

To a solution of 6-bromo-3-(2,6-dibenzyloxy-3-pyridyl)-1H-benzimidazol-2-one (1.3 g, 2.6 mmol) and 2-iodopropane (2.6 mL, 26 mmol) in N,N-dimethyl formamide (15 mL) was added cesium carbonate (2.53 g, 7.8 mmol). The mixture was stirred at 60° C. for 16 h, then diluted with water (30 mL) and extracted with ethyl acetate (100 mL×3). The combined organic layers were washed with brine (20 mL), dried over anhydrous sodium sulfate, filtered and concentrated under reduced pressure. The residue was purified by silica gel column chromatography (petroleum ether/ethyl acetate=1/0 to 5/1) to afford the title compound (1 g, 71%) as a brown oil. MS (ESI) m/z: 544.2 [M+H]⁺.

### Step 16: Preparation of 1-(2,6-dibenzyloxy-3-pyridyl)-5-[4-(dimethoxy methyl)-1-piperidyl]-3-isopropyl-benzimidazol-2-one

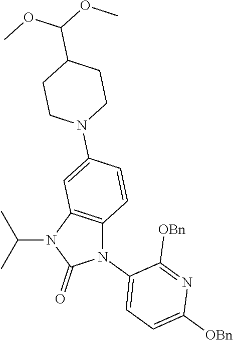 [SMILES: COC(OC)C1CCN(c2ccc3c(c2)n(C(C)C)c(=O)n3-c2ccc(OCc3ccccc3)nc2OCc2ccccc2)CC1]

To a solution of 4-(dimethoxymethyl)piperidine (468 mg, 3 mmol) and 5-bromo-1-(2,6-dibenzyloxy-3-pyridyl)-3-isopropyl-benzimidazol-2-one (1.6 g, 3 mmol) in dioxane (20 mL) were added Xphos Pd G2 (231 mg, 0.3 mmol) and cesium carbonate (2.88 g, 9 mmol). The mixture was stirred at 100° C. for 16 h under nitrogen atmosphere. The reaction mixture was filtered and concentrated. The residue was purified by silica gel column chromatography (petroleum ether/ethyl acetate=3/1 to 1/1) to afford the title compound (1.6 g, 87%) as an orange oil. MS (ESI) m/z: 623.4 [M+H]⁺.

### Step 17: Preparation of 3-[5-[4-(dimethoxymethyl)-1-piperidyl]-3-isopropyl-2-oxo-benzimidazol-1-yl]piperidine-2,6-dione

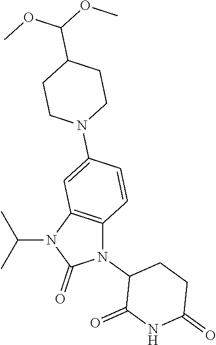 [SMILES: COC(OC)C1CCN(c2ccc3c(c2)n(C(C)C)c(=O)n3C2CCC(=O)NC2=O)CC1]

To a solution of 1-(2,6-dibenzyloxy-3-pyridyl)-5-[4-(dimethoxymethyl)-1-piperidyl]-3-isopropyl-benzimidazol-2-one (1.35 g, 2 mmol) in tetrahydrofuran (10 mL) was added 10% palladium on carbon (300 mg) under nitrogen. The suspension was degassed and purged with hydrogen several times. The mixture was stirred under hydrogen at 50° C. for 16 h. The reaction mixture was filtered and concentrated under reduced pressure to afford the title compound (800 mg, 83%) as a green solid, which was used in the next step without further purification. MS (ESI) m/z: 445.3 [M+H]⁺.

### Step 18: Preparation of 1-[1-(2,6-dioxo-3-piperidyl)-3-isopropyl-2-oxo-benzimidazol-5-yl]piperidine-4-carbaldehyde

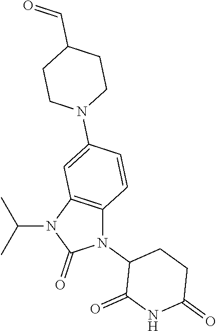 [SMILES: CC(C)n1c(=O)n(C2CCC(=O)NC2=O)c2ccc(N3CCC(C=O)CC3)cc21]

To a solution of 3-[5-[4-(dimethoxymethyl)-1-piperidyl]-3-isopropyl-2-oxo-benzimidazol-1-yl]piperidine-2,6-dione (800 mg, 1.8 mmol) in dichloromethane (10 mL) was added trifluoroacetic acid (0.13 mL, 1.8 mmol). The mixture was stirred at 25° C. for 1 h, then concentrated under reduced pressure. The residue was triturated with ethyl acetate/methyl tert-butyl ether (1/10, 100 mL) and then filtered to afford the title compound (700 mg, 98%) as a green solid. MS (ESI) m/z: 417.2 [M+18]⁺.

### Step 19: Preparation of 3-[5-[4-[[4-[6-[[7-(7-fluoroimidazo[1,2-a]pyridin-3-yl)-3-oxo-isoindolin-4-yl]amino]-2-methyl-3-pyridyl]piperazin-1-yl]methyl]-1-piperidyl]-3-isopropyl-2-oxo-benzimidazol-1-yl]piperidine-2,6-dione

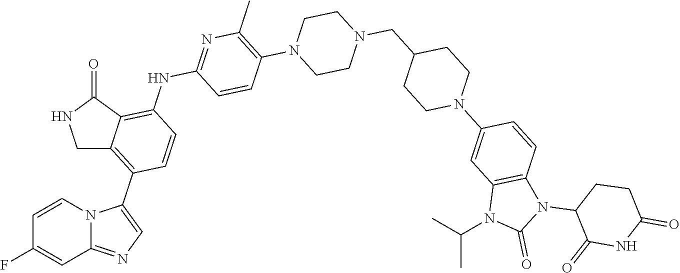 [SMILES: Cc1nc(Nc2ccc(-c3cnc4cc(F)ccn34)c3c2C(=O)NC3)ccc1N1CCN(CC2CCN(c3ccc4c(c3)n(C(C)C)c(=O)n4C3CCC(=O)NC3=O)CC2)CC1]

To a mixture of 4-(7-fluoroimidazo[1,2-a]pyridin-3-yl)-7-[(6-methyl-5-piperazin-1-yl-2-pyridyl)amino]isoindolin-1-one (75 mg, 0.1 mmol, 2 trifluoroacetate) in dichloromethane (5 mL) was added 4-methylmorpholine (0.06 mL, 0.6 mmol). The mixture was stirred at 25° C. for 0.5 h, followed by the addition of 1-[1-(2,6-dioxo-3-piperidyl)-3-isopropyl-2-oxo-benzimidazol-5-yl]piperidine-4-carbaldehyde (57 mg, 0.1 mmol), the mixture was stirred at 25° C. for 0.5 h before the addition of sodium triacetoxyborohydride (70 mg, 0.3 mmol). The reaction mixture was stirred at 25° C. for 2 h, then poured into water (10 mL) and extracted with ethyl acetate (5 mL×3). The combined organic phase was washed with brine (15 mL), dried over anhydrous sodium sulfate, filtered, and concentrated under reduced pressure. The residue was purified by semi-preparative reverse phase column chromatography (1%-30% acetonitrile in water (formic acid) over 10 min) to afford the title compound (53 mg, 57%) as a yellow solid. MS (ESI) m/z: 840.5 [M+H]⁺; ¹H NMR (400 MHz, DMSO-d₆) δ 11.11-11.00 (m, 1H), 9.98-9.91 (m, 1H), 8.79 (s, 1H), 8.66 (d, J=8.4 Hz, 1H), 8.49-8.41 (m, 1H), 7.82 (s, 1H), 7.71 (d, J=8.4 Hz, 1H), 7.56-7.44 (m, 2H), 6.99-6.83 (m, 4H), 6.67-6.60 (m, 1H), 5.30-5.22 (m, 1H), 4.59 (td, J=6.8, 13.6 Hz, 1H), 4.38 (s, 2H), 3.56 (d, J=10.8 Hz, 2H), 2.95-2.75 (m, 6H), 2.65-2.55 (m, 7H), 2.46 (s, 3H), 2.26 (d, J=7.2 Hz, 2H), 2.03-1.94 (m, 1H), 1.87-1.80 (m, 2H), 1.73-1.61 (m, 1H), 1.45 (d, J=6.8 Hz, 6H), 1.32-1.23 (m, 2H)

### Example 2: Exemplary Synthesis of 3-(5-(4-((4-(6-((7-(8-fluoroimidazo[1,2-a]pyridin-3-yl)-3-oxoisoindolin-4-yl)amino)-2-methylpyridin-3-yl)piperazin-1-yl)methyl)piperidin-1-yl)-3-isopropyl-2-oxo-2,3-dihydro-1H-benzo[d]imidazol-1-yl)piperidine-2,6-dione

### Step 1: Preparation of tert-butyl 4-(6-((2-(2,4-dimethoxybenzyl)-7-(8-fluoroimidazo[1,2-a]pyridin-3-yl)-3-oxoisoindolin-4-yl)amino)-2-methylpyridin-3-yl)piperazine-1-carboxylate

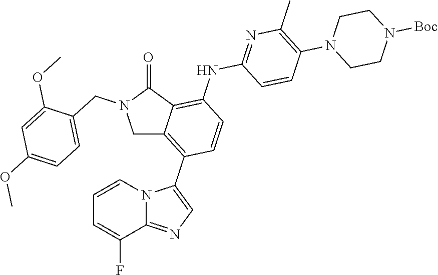 [SMILES: COc1ccc(CN2Cc3c(-c4cnc5c(F)cccn45)ccc(Nc4ccc(N5CCN(C(=O)OC(C)(C)C)CC5)c(C)n4)c3C2=O)c(OC)c1]

A mixture of 7-chloro-2-[(2,4-dimethoxyphenyl)methyl]-4-(8-fluoroimidazo[1,2-a]pyridin-3-yl)isoindolin-1-one (260 mg, 0.6 mmol), tert-butyl 4-(6-amino-2-methyl-3-pyridyl)piperazine-1-carboxylate (168 mg, 0.6 mmol), Ruphos Pd G3 (48 mg, 0.06 mmol) and cesium carbonate (469 mg, 1.4 mmol) in dioxane (5 mL) was degassed and purged with nitrogen thrice, the mixture was stirred at 100° C. for 12 h under nitrogen atmosphere. The mixture was diluted with water (10 mL) and extracted with dichloromethane (10 mL×3). The combined organic layers were washed with brine (10 mL×2), dried over anhydrous sodium sulfate, filtered and concentrated. The residue was purified by column chromatography (petroleum ether/ethyl acetate=1/0 to 1/1), the crude product was further purified by prep-HPLC (58%-88% acetonitrile in water (formic acid) over 15 min) to afford the title compound (200 mg, 49%) as a yellow solid. MS (ESI) m/z: 708.3 [M+H]⁺; ¹H NMR (400 MHz, DMSO-d₆) δ 9.96 (s, 1H), 8.76 (d, J=8.4 Hz, 1H), 8.25 (d, J=6.8 Hz, 1H), 7.87 (s, 1H), 7.75 (d, J=8.4 Hz, 1H), 7.52 (d, J=8.4 Hz, 1H), 7.30-7.20 (m, 1H), 7.15 (d, J=8.4 Hz, 1H), 6.96-6.87 (m, 2H), 6.60 (d, J=2.4 Hz, 1H), 6.51 (dd, J=2.4, 8.4 Hz, 1H), 4.64 (s, 2H), 4.41 (s, 2H), 3.81 (s, 3H), 3.77 (s, 3H), 3.52 (s, 4H), 2.83 (t, J=4.8 Hz, 4H), 2.52 (s, 3H), 1.47 (s, 9H).

### Step 2: Preparation of 4-(8-fluoroimidazo[1,2-a]pyridin-3-yl)-7-((6-methyl-5-(piperazin-1-yl)pyridin-2-yl)amino)isoindolin-1-one

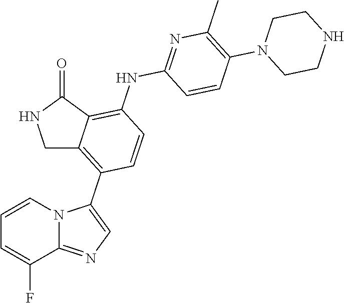 [SMILES: Cc1nc(Nc2ccc(-c3cnc4c(F)cccn34)c3c2C(=O)NC3)ccc1N1CCNCC1]

To a solution of tert-butyl 4-[6-[[2-[(2,4-dimethoxyphenyl)methyl]-7-(8-fluoroimidazo[1,2-a]pyridin-3-yl)-3-oxo-isoindolin-4-yl]amino]-2-methyl-3-pyridyl]piperazine-1-carboxylate (200 mg, 0.27 mmol, formate) in dichloromethane (2 mL) was added trifluoroacetic acid (1 mL). The mixture was stirred at 20° C. for 1 h, followed by another addition of trifluoromethanesulfonic acid (1 mL), the mixture was stirred at 40° C. for 16 h. The mixture was diluted with water (10 mL), the pH of the mixture was adjusted to 8 with sodium bicarbonate. The resulting mixture was extracted with dichloromethane (10 mL×2). The combined organic layers were washed with brine (10 mL×2), dried over anhydrous sodium sulfate, filtered and concentrated. The residue was purified by preparative HPLC (2%-32% acetonitrile in water (formic acid) over 15 min) to afford the title compound (40 mg, 33%) as a white solid. MS (ESI) m/z: 458.1 [M+H]⁺.

### Step 3: Preparation of 3-(5-(4-((4-(6-((7-(8-fluoroimidazo[1,2-a]pyridin-3-yl)-3-oxoisoindolin-4-yl)amino)-2-methylpyridin-3-yl)piperazin-1-yl)methyl)piperidin-1-yl)-3-isopropyl-2-oxo-2,3-dihydro-1H-benzo[d]imidazol-1-yl)piperidine-2,6-dione

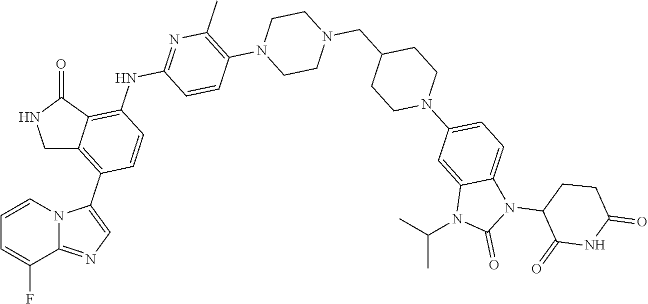 [SMILES: Cc1nc(Nc2ccc(-c3cnc4c(F)cccn34)c3c2C(=O)NC3)ccc1N1CCN(CC2CCN(c3ccc4c(c3)n(C(C)C)c(=O)n4C3CCC(=O)NC3=O)CC2)CC1]

To a solution of 4-(8-fluoroimidazo[1,2-a]pyridin-3-yl)-7-[(6-methyl-5-piperazin-1-yl-2-pyridyl)amino]isoindolin-1-one (40 mg, 0.09 mmol) and 1-[l-(2,6-dioxo-3-piperidyl)-3-isopropyl-2-oxo-benzimidazol-5-yl]piperidine-4-carbaldehyde (35 mg, 0.09 mmol) in dichloromethane (1 mL) and dimethyl sulfoxide (0.5 mL) was added 4-methylmorpholine (8.84 mg, 0.09 mmol), the mixture was stirred at 20° C. for 1 h. To the above mixture were added acetic acid (11 mg, 0.2 mmol) and sodium triacetoxyborohydride (37 mg, 0.2 mmol). The reaction mixture was stirred at 20° C. for 2 h, then filtered and concentrated under reduced pressure. The residue was purified by preparative HPLC (5%-35% acetonitrile in water (formic acid) over 10 min) to afford the title compound (32.50 mg, 44%) as a white solid. MS (ESI) m/z: 840.6 [M+H]⁺; ¹H NMR (400 MHz, DMSO-d₆) δ 11.07 (s, 1H), 9.95 (s, 1H), 8.81 (s, 1H), 8.67 (d, J=8.4 Hz, 1H), 8.25 (d, J=6.8 Hz, 1H), 7.89 (s, 1H), 7.72 (d, J=8.4 Hz, 1H), 7.47 (d, J=8.4 Hz, 1H), 7.21 (dd, J=7.6, 11.2 Hz, 1H), 6.98-6.82 (m, 4H), 6.67-6.60 (m, 1H), 5.27 (dd, J=5.2, 12.8 Hz, 1H), 4.60-4.50 (m, 1H), 4.40 (s, 2H), 3.56 (d, J=12.4 Hz, 2H), 2.93-2.80 (m, 5H), 2.71-2.53 (m, 8H), 2.46 (s, 3H), 2.27 (d, J=7.2 Hz, 2H), 2.03-1.92 (m, 1H), 1.83 (d, J=12.0 Hz, 2H), 1.74-1.60 (m, 1H), 1.45 (d, J=6.8 Hz, 6H), 1.34-1.22 (m, 2H).

### Exemplification of Method B

### Example 3: Exemplary Synthesis of 3-[4-chloro-5-[4-[4-[6-[[7-(7-fluoroimidazo[1,2-a]pyridin-3-yl)-3-oxo-isoindolin-4-yl]amino]-2-methyl-3-pyridyl]-1-piperidyl]-1-piperidyl]-1-oxo-isoindolin-2-yl]piperidine-2,6-dione

### Step 1: Preparation of tert-butyl 4-(6-amino-2-methyl-3-pyridyl)-3,6-dihydro-2H-pyridine-1-carboxylate

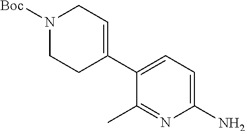 [SMILES: Cc1nc(N)ccc1C1=CCN(C(=O)OC(C)(C)C)CC1]

A solution of 5-bromo-6-methyl-pyridin-2-amine (5.0 g, 27 mmol), tert-butyl 4-(4,4,5,5-tetramethyl-1,3,2-dioxaborolan-2-yl)-3,6-dihydro-2H-pyridine-1-carboxylate (9.09 g, 29 mmol) and tritripotassium phosphate (14.19 g, 67 mmol) in dioxane (200 mL) and water (20 mL) was degassed and purged with nitrogen several times, then Xphos Pd G2 (1.05 g, 1.3 mmol) was added to the mixture. The reaction mixture was stirred at 140° C. for 4 h under nitrogen atmosphere. The mixture was cooled to 20° C., diluted with brine (50 mL) and extracted with dichloromethane (30 mL×3). The combined organic phase was dried over anhydrous sodium sulfate, filtered, and concentrated under reduced pressure. The residue was purified by column chromatography (petroleum ether/ethyl acetate=1/0 to 0/1) to afford the title compound (6.53 g, 84%) as a brown solid. MS (ESI) m/z: 290.3 [M+H]⁺; ¹H NMR (400 MHz, DMSO-d₆) δ 7.08 (d, J=8.4 Hz, 1H), 6.23 (d, J=8.4 Hz, 1H), 5.76 (s, 2H), 5.49 (s, 1H), 3.94 (s, 2H), 3.49 (t, J=5.6 Hz, 2H), 3.17 (d, J=5.2 Hz, 2H), 2.20 (s, 3H), 1.42 (s, 9H).

### Step 2: Preparation of tert-butyl 4-(6-amino-2-methyl-3-pyridyl)piperidine-1-carboxylate

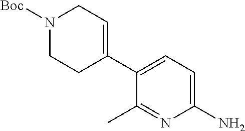 [SMILES: Cc1nc(N)ccc1C1=CCN(C(=O)OC(C)(C)C)CC1]

To a solution of tert-butyl 4-(6-amino-2-methyl-3-pyridyl)-3,6-dihydro-2H-pyridine-1-carboxylate (6.5 g, 22 mmol) in methanol (70 mL) was added 10% palladium on carbon (1 g), the mixture was degassed and purged with hydrogen several times. The reaction mixture was stirred at 25° C. under hydrogen (50 psi) for 12 h, then filtered and concentrated under reduced pressure to afford the title compound (8.9 g, crude) as white solid. MS (ESI) m/z: 292.2 [M+H]⁺; ¹H NMR (400 MHz, DMSO-d₆) δ 7.17 (d, J=8.4 Hz, 1H), 6.24 (d, J=8.4 Hz, 1H), 5.56 (s, 2H), 4.05 (d, J=10.0 Hz, 2H), 2.87-2.65 (m, 3H), 2.27 (s, 3H), 1.60 (d, J=12.8 Hz, 2H), 1.41 (s, 9H), 1.38-1.34 (m, 2H).

### Step 3: Preparation of methyl 3-chloro-2-methylbenzoate

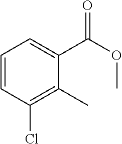

To a solution of 3-chloro-2-methyl-benzoic acid (5.0 g, 29 mmol) in dimethylformamide (50 mL) were added potassium carbonate (4.05 g, 29 mmol) and iodomethane (5.5 mL, 88 mmol). The mixture was stirred at 25° C. for 16 h. The reaction mixture was diluted with water (150 mL) and extracted with ethyl acetate (150 mL×3). The combined organic layers were washed with brine (150 mL×3), dried over sodium sulfate anhydrous, filtered, and concentrated under reduced pressure to afford the title compound (5.3 g, crude) as a light brown liquid.

### Step 4: Preparation of methyl 2-(bromomethyl)-3-chlorobenzoate

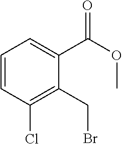

To a solution of methyl 3-chloro-2-methyl-benzoate (2.0 g, 11 mmol) in carbon tetrachloride (20 mL) was added benzoyl peroxide (4.72 mg, 0.02 mmol) and N-bromosuccinimide (2.10 g, 11.81 mmol). The mixture was stirred at 90° C. for 4 h. The combined reaction mixture was poured into ice-water (100 mL). The reaction mixture was diluted with water (50 mL) and extracted with ethyl acetate (100 mL×3). The combined organic layers were washed with brine (50 mL×3), dried over sodium sulfate anhydrous, filtered, and concentrated under reduced pressure to afford the title compound (2.5 g, crude) as a white solid, which was used directly in the next step.

### Step 5: Preparation of 4-chloroisoindolin-1-one

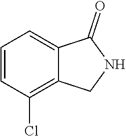 [SMILES: O=C1NCc2c(Cl)cccc21]

To a solution of methyl 2-(bromomethyl)-3-chloro-benzoate (2.5 g, 9.5 mmol) in methanol (20 mL) was bubbled with ammonia gas (161 mg, 9.5 mmol) for 1 h at 0° C. The mixture was stirred at 25° C. for 16 h, then poured into ice-water (100 mL). The mixture was filtered, the filtrate solution was concentrated under reduced pressure to afford the title compound (1.25 g, crude) as a white solid, which was used in the next step without further purification. ¹H NMR (400 MHz, DMSO-d₆) δ 8.80 (s, 1H), 7.73-7.62 (m, 2H), 7.56-7.51 (m, 1H), 4.38 (s, 2H).

### Step 6: Preparation of 4-chloro-7-nitroisoindolin-1-one

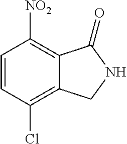 [SMILES: O=C1NCc2c(Cl)ccc([N+](=O)[O-])c21]

To a solution of 4-chloroisoindolin-1-one (1.25 g, 7.5 mmol) in sulfuric acid (10 mL) was dropwise added nitric acid (1.36 g, 15 mmol, 68%) at −10° C. The reaction mixture was stirred at 25° C. for 4 h, then poured into ice water (100 mL). The suspension was filtered and washed with water (200 mL). The filter cake was dried to afford the title compound (1.43 g, 90%) as a yellow solid. ¹H NMR (400 MHz, DMSO-d₆) δ 9.18 (s, 1H), 8.17-7.59 (m, 2H), 4.46 (s, 2H).

### Step 7: Preparation of 7-amino-4-chloroisoindolin-1-one

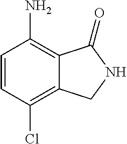 [SMILES: Nc1ccc(Cl)c2c1C(=O)NC2]

To a solution of 4-chloro-7-nitro-isoindolin-1-one (1.43 g, 6.7 mmol) in ethanol (10 mL) and water (2 mL) were added iron (1.88 g, 34 mmol) and ammonium chloride (2.16 g, 40 mmol). The mixture was stirred at 70° C. for 2 h, then poured into ice-water (100 mL). The mixture was filtered, the filter cake was dried to afford the title compound (1 g, 81%) as a yellow solid. ¹H NMR (400 MHz, DMSO-d₆) δ 8.54-8.25 (m, 1H), 7.21 (t, J=17.2 Hz, 1H), 6.70-6.47 (m, 1H), 6.17 (d, J=1.2 Hz, 2H), 4.21 (s, 2H).

### Step 8: Preparation of 7-bromo-4-chloroisoindolin-1-one

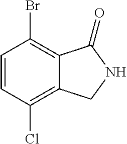 [SMILES: O=C1NCc2c(Cl)ccc(Br)c21]

To a solution of 7-amino-4-chloro-isoindolin-1-one (600 mg, 3.3 mmol) in hydrogen bromide (5.25 g, 26 mmol, 40%) was added sodium nitrite (453 mg, 6.6 mmol) in water (3.5 mL) at −10° C., the mixture was stirred at −10° C. for 60 min, followed by the addition of cuprous bromide (516 mg, 3.6 mmol). The reaction mixture was stirred at 80° C. for 40 min, then filtered. The filter cake was triturated with water (150 mL) to afford the title compound (620 mg, 77%) as a white solid. MS (ESI) m/z: 245.9 [M+H]⁺.

### Step 9: Preparation of tert-butyl 7-bromo-4-chloro-1-oxoisoindoline-2-carboxylate

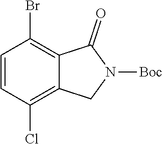 [SMILES: CC(C)(C)OC(=O)N1Cc2c(Cl)ccc(Br)c2C1=O]

To a solution of 7-bromo-4-chloro-isoindolin-1-one (620 mg, 2.5 mmol) in tetrahydrofuran (8 mL) at 0° C. were added dimethylaminopyridine (368 mg, 3 mmol) and di-tert-butyl decarbonate (823 mg, 3.8 mmol). The mixture was stirred at 25° C. for 4 h, then diluted with water (50 mL) and extracted with ethyl acetate (50 mL×3). The combined organic layers were washed with brine (50 mL×3), dried over sodium sulfate anhydrous, filtered, and concentrated under reduced pressure. The residue was purified by silica gel chromatography (petroleum ether:ethyl acetate=50:1 to 5:1) to afford the title compound (770 mg, 88%) as a white solid. MS (ESI) m/z: 291.9 [M−56+H]⁺; ¹H NMR (400 MHz, DMSO-d₆) δ 7.78-7.68 (m, 2H), 4.69 (s, 2H), 1.53 (s, 9H).

### Step 10: Preparation of tert-butyl 7-[[5-(1-tert-butoxycarbonyl-4-piperidyl)-6-methyl-2-pyridyl]amino]-4-chloro-1-oxo-isoindoline-2-carboxylate

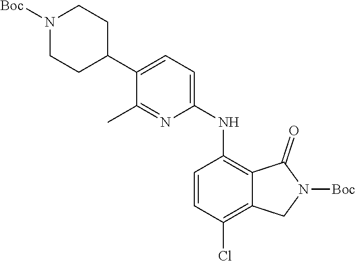 [SMILES: Cc1nc(Nc2ccc(Cl)c3c2C(=O)N(C(=O)OC(C)(C)C)C3)ccc1C1CCN(C(=O)OC(C)(C)C)CC1]

To a solution of tert-butyl 4-(6-amino-2-methyl-3-pyridyl)piperidine-1-carboxylate (500 mg, 1.7 mmol) and tert-butyl 7-bromo-4-chloro-1-oxo-isoindoline-2-carboxylate (595 mg, 1.7 mmol) in dioxane (10 mL) were added 4,5-bis(diphenylphosphino)-9,9-dimethylxanthene (99 mg, 0.2 mmol), cesium carbonate (1.12 g, 3.4 mmol) and tris(dibenzylideneacetone)dipalladium (157 mg, 0.17 mmol). The reaction mixture was degassed and purged with nitrogen, then stirred at 90° C. for 2 h under nitrogen. The mixture was diluted with water (50 mL) and extracted with ethyl acetate (2×100 mL). The combined organic layers were washed with brine (50 mL), dried over anhydrous sodium sulfate, filtered, and concentrated under reduced pressure. The residue was purified by flash silica gel chromatography (0-40% ethyl acetate/petroleum ether). The residue was purified by preparative TLC (ethyl acetate:petroleum ether=1:4) to afford the title compound (469 mg, 49%) as a yellow oil. MS (ESI) m/z: 557.2 [M+H]⁺.

### Step 11: Preparation of tert-butyl 7-[[5-(1-tert-butoxycarbonyl-4-piperidyl)-6-methyl-2-pyridyl]amino]-1-oxo-4-(4,4,5,5-tetramethyl-1,3,2-dioxaborolan-2-yl)isoindoline-2-carboxylate

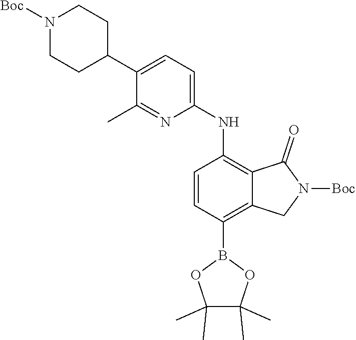 [SMILES: Cc1nc(Nc2ccc(B3OC(C)(C)C(C)(C)O3)c3c2C(=O)N(C(=O)OC(C)(C)C)C3)ccc1C1CCN(C(=O)OC(C)(C)C)CC1]

To a solution of tert-butyl 7-[[5-(1-tert-butoxycarbonyl-4-piperidyl)-6-methyl-2-pyridyl]amino]-4-chloro-1-oxo-isoindoline-2-carboxylate (469 mg, 0.8 mmol), potassium acetate (165 mg, 1.7 mmol) and bis(pinacolato)diboron (257 mg, 1 mmol) in dioxane (5 mL) was added Xphos Pd G2 (66 mg, 0.08 mmol). The reaction mixture was degassed and purged with nitrogen, then stirred at 90° C. for 2 h under nitrogen. The mixture was directly filtered through a pad of celite and washed with ethyl acetate. The filtrate solution was concentrated under reduced pressure to afford the title compound (512 mg, crude) as an orange solid. MS (ESI) m/z: 649.3 [M+H]⁺.

### Step 12: Preparation of 7-fluoroimidazo[1,2-a]pyridine

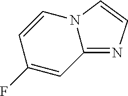

To a solution of 4-fluoropyridin-2-amine (1 g, 9 mmol) in ethanol (9 mL) were added sodium bicarbonate (1.50 g, 18 mmol) and 2-chloroacetaldehyde (7.50 mL, 47 mmol, 40%). The mixture was stirred at 60° C. for 4 h. The reaction mixture was diluted with water (50 mL) and extracted with ethyl acetate (50 mL×3). The combined organic layers were washed with brine (50 mL×3), dried over sodium sulfate anhydrous, filtered, and concentrated under reduced pressure. The residue was purified by silica gel chromatography (petroleum ether:ethyl acetate=10:1 to 1:1) to give the title compound (0.9 g, 74%) as a brown oil. ¹H NMR (400 MHz, DMSO-d₆) δ 8.62 (dd, J=6.4, 6.8 Hz, 1H), 7.93 (s, 1H), 7.55 (d, J=1.2 Hz, 1H), 7.41 (dd, J=2.4, 10.2 Hz, 1H), 6.96 (dt, J=2.8, 7.6 Hz, 1H).

### Step 13: Preparation of 7-fluoro-3-iodoimidazo[1,2-a]pyridine

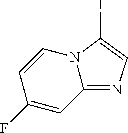 [SMILES: Fc1ccn2c(I)cnc2c1]

To a solution of 7-fluoroimidazo[1,2-a]pyridine (800 mg, 5.9 mmol) in chloroform (10 mL) was added NIS (1.45 g, 6.5 mmol). The mixture was stirred at 25° C. for 3 h. The reaction mixture was diluted with water (50 mL) and extracted with ethyl acetate (50 mL×3). The combined organic layers were washed with brine (50 mL×3), dried over anhydrous sodium sulfate, filtered, and concentrated under reduced pressure. The residue was purified by silica gel chromatography (petroleum ether:ethyl acetate=10:1 to 3:1) to afford the title compound (770 mg, 50%) as a brown solid. ¹H NMR (400 MHz, DMSO-d₆) δ 8.41 (dd, J=5.6, 7.2 Hz, 1H), 7.74 (s, 1H), 7.56 (dd, J=2.4, 9.6 Hz, 1H), 7.13 (dt, J=2.4, 7.6 Hz, 1H).

### Step 14: Preparation of tert-butyl 7-[[5-(1-tert-butoxycarbonyl-4-piperidyl)-6-methyl-2-pyridyl]amino]-4-(7-fluoroimidazo[1,2-a]pyridin-3-yl)-1-oxo-isoindoline-2-carboxylate

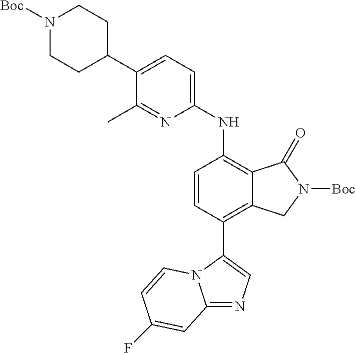 [SMILES: Cc1nc(Nc2ccc(-c3cnc4cc(F)ccn34)c3c2C(=O)N(C(=O)OC(C)(C)C)C3)ccc1C1CCN(C(=O)OC(C)(C)C)CC1]

A mixture of tert-butyl 7-[[5-(1-tert-butoxycarbonyl-4-piperidyl)-6-methyl-2-pyridyl]amino]-1-oxo-4-(4,4,5,5-tetramethyl-1,3,2-dioxaborolan-2-yl)isoindoline-2-carboxylate (400 mg, 0.6 mmol), 7-fluoro-3-iodo-imidazo[1,2-a]pyridine (170 mg, 0.7 mmol), tritripotassium phosphate (393 mg, 1.9 mmol), Xphos Pd G2 (49 mg, 0.06 mmol) and dicyclohexyl-[2-(2,4,6-triisopropylphenyl)phenyl]phosphane (15 mg, 0.03 mmol) in dioxane (10 mL) and water (2 mL) was degassed and purged with nitrogen several times, and the mixture was stirred at 100° C. for 3 h under nitrogen atmosphere. The reaction mixture was filtered and concentrated under reduced pressure. The residue was purified by preparative TLC (petroleum ether:ethyl acetate=0:1) to afford the title compound (196 mg, 48%) as a yellow solid. MS (ESI) m/z: 657.2 [M+H]⁺.

### Step 15: Preparation of 4-(7-fluoroimidazo[1,2-a]pyridin-3-yl)-7-[[6-methyl-5-(4-piperidyl)-2-pyridyl]amino]isoindolin-1-one

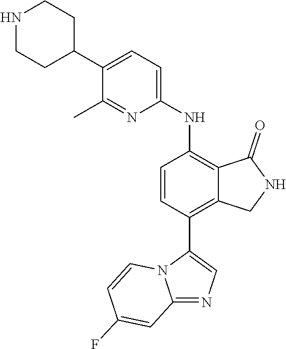 [SMILES: Cc1nc(Nc2ccc(-c3cnc4cc(F)ccn34)c3c2C(=O)NC3)ccc1C1CCNCC1]

To a solution of tert-butyl 7-[[5-(1-tert-butoxycarbonyl-4-piperidyl)-6-methyl-2-pyridyl]amino]-4-(7-fluoroimidazo[1,2-a]pyridin-3-yl)-1-oxo-isoindoline-2-carboxylate (70 mg, 0.11 mmol) in dichloromethane (3 mL) was added 4 M hydrochloric acid in dioxane (1.8 mL). The reaction solution was stirred at 25° C. for 1 h, then concentrated under reduced pressure to afford the title compound (50 mg, 95%, hydrochloride) as a yellow solid. MS (ESI) m/z: 457.2 [M+H]⁺.

### Step 16: Preparation of 4-chloro-5-fluoro-3-hydroxy-3H-isobenzofuran-1-one

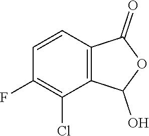 [SMILES: O=C1OC(O)c2c1ccc(F)c2Cl]

To a solution of N-isopropylpropan-2-amine (9.7 mL, 69 mmol) in tetrahydrofuran (20 mL) was added n-butyllithium (2.5 M, 26.4 mL) at −78° C. The solution was stirred at −30° C. for 1 h, followed by the addition of 3-chloro-4-fluoro-benzoic acid (5.0 g, 29 mmol) in tetrahydrofuran (50 mL). The mixture was stirred at −78° C. for 2 h, then N,N-dimethylformamide (4.4 mL, 57 mmol) was added and the resulting mixture was stirred at 20° C. for 12 h. The reaction mixture was diluted with 4 N aqueous hydrochloric acid solution until pH=3, and the mixture was extracted with ethyl acetate (50 mL×3). The combined organic phase was washed with brine (150 mL), dried over anhydrous sodium sulfate, filtered, and concentrated under reduced pressure to afford the title compound (5 g, 86%) as a yellow oil, which was used in the next step directly.

### Step 17: Preparation of 3-(4-chloro-5-fluoro-1-oxo-isoindolin-2-yl) piperidine-2,6-dione

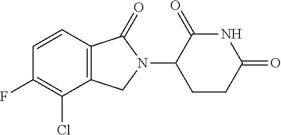 [SMILES: O=C1CCC(N2Cc3c(ccc(F)c3Cl)C2=O)C(=O)N1]

To a solution of 4-chloro-5-fluoro-3-hydroxy-3H-isobenzofuran-1-one (500 mg, 2.5 mmol) and 3-aminopiperidine-2,6-dione (488 mg, 3 mmol, hydrochloride) in methanol (10 mL) and acetic acid (1 mL) was added sodium acetate (607 mg, 7.4 mmol). The mixture was stirred at 50° C. for 1 h, followed by the addition of 2-methylpyridine borane (528 mg, 4.9 mmol). The reaction mixture was stirred at 50° C. for 1 h, then diluted with water (50 mL) and extracted with tetrahydrofuran (15 mL×3). The combined organic layer was washed with brine (50 mL), dried over sodium sulfate, filtered, and concentrated under reduced pressure. The residue was purified by prep-TLC (dichloromethane:methanol=10:1) to afford the title compound (130 mg, 18%) as a white solid. ¹H NMR (400 MHz, DMSO-d₆) δ 11.01 (s, 1H), 7.77 (dd, J=4.4, 8.4 Hz, 1H), 7.58 (t, J=9.2 Hz, 1H), 5.13 (dd, J=4.8, 13.2 Hz, 1H), 4.62-4.45 (m, 1H), 4.44-4.29 (m, 1H), 3.00-2.83 (m, 1H), 2.60 (d, J=17.6 Hz, 1H), 2.49-2.38 (m, 1H), 2.10-1.95 (m, 1H).

### Step 18: Preparation of 3-[4-chloro-5-(1,4-dioxa-8-azaspiro[4.5]decan-8-yl)-1-oxo-isoindolin-2-yl]piperidine-2,6-dione

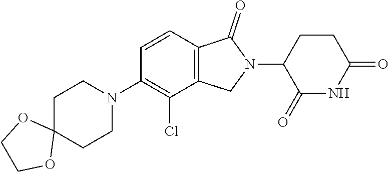 [SMILES: O=C1CCC(N2Cc3c(ccc(N4CCC5(CC4)OCCO5)c3Cl)C2=O)C(=O)N1]

To a solution of 3-(4-chloro-5-fluoro-1-oxo-isoindolin-2-yl)piperidine-2,6-dione (2 g, 7 mmol) in dimethyl sulfoxide (20 mL) were added N,N-diisopropylethylamine (5.8 mL, 34 mmol) and 1,4-dioxa-8-azaspiro[4.5]decane (1.0 mL, 8 mmol). The mixture was stirred at 130° C. for 12 h, then poured into water (200 mL) and extracted with ethyl acetate (50 mL×3). The combined organic phase was washed with brine (150 mL), dried over anhydrous sodium sulfate, filtered, and concentrated under reduced pressure. The crude product was triturated with ethyl acetate (15 mL) to afford the title compound (1.8 g, 63%) as a white solid. MS (ESI) m/z: 420.1 [M+H]⁺; ¹H NMR (400 MHz, DMSO-d₆) δ 10.98 (s, 1H), 7.63 (d, J=8.0 Hz, 1H), 7.30 (d, J=8.0 Hz, 1H), 5.09 (dd, J=5.2, 13.2 Hz, 1H), 4.45-4.37 (m, 1H), 4.29-4.22 (m, 1H), 3.93 (s, 4H), 3.17-3.09 (m, 4H), 2.68-2.52 (m, 2H), 2.51-2.50 (m, 2H), 1.81 (t, J=5.6 Hz, 4H).

### Step 19: Preparation of 3-[4-chloro-1-oxo-5-(4-oxo-1-piperidyl)isoindolin-2-yl]piperidine-2,6-dione

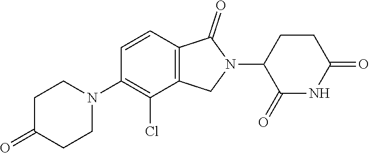 [SMILES: O=C1CCN(c2ccc3c(c2Cl)CN(C2CCC(=O)NC2=O)C3=O)CC1]

To a solution of 3-[4-chloro-5-(1,4-dioxa-8-azaspiro[4.5]decan-8-yl)-1-oxo-isoindolin-2-yl]piperidine-2,6-dione (1.93 g, 4.6 mmol) in tetrahydrofuran (20 mL) was added 2 M sulfuric acid (20 mL). The mixture was stirred at 50° C. for 1 h, then concentrated under reduced pressure. The residue was diluted with saturated sodium bicarbonate solution to adjust the pH to 8, the mixture was extracted with ethyl acetate (10 mL×3). The combined organic layers were washed with brine (10 mL×3), dried over sodium sulfate anhydrous, filtered, and concentrated under reduced pressure to afford the title compound (1.08 g, 62%) as a white solid. MS (ESI) m/z: 376.0 [M+H]⁺; ¹H NMR (400 MHz, DMSO-d₆) δ 10.98 (s, 1H), 7.70-7.56 (m, 1H), 7.37-7.24 (m, 1H), 5.10 (dd, J=5.2, 13.2 Hz, 1H), 4.52-4.24 (m, 2H), 3.41 (t, J=5.6 Hz, 4H), 2.97-2.84 (m, 1H), 2.64-2.59 (m, 1H), 2.56 (t, J=6.0 Hz, 4H), 2.49-2.38 (m, 2H).

### Step 20: Preparation of 3-[4-chloro-5-[4-[4-[6-[[7-(7-fluoroimidazo[1,2-a]pyridin-3-yl)-3-oxo-isoindolin-4-yl]amino]-2-methyl-3-pyridyl]-1-piperidyl]-1-piperidyl]-1-oxo-isoindolin-2-yl]piperidine-2,6-dione

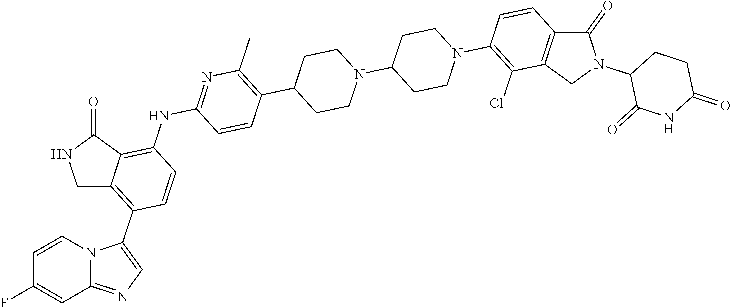 [SMILES: Cc1nc(Nc2ccc(-c3cnc4cc(F)ccn34)c3c2C(=O)NC3)ccc1C1CCN(C2CCN(c3ccc4c(c3Cl)CN(C3CCC(=O)NC3=O)C4=O)CC2)CC1]

To a solution of 3-[4-chloro-1-oxo-5-(4-oxo-1-piperidyl)isoindolin-2-yl]piperidine-2,6-dione (70 mg, 0.2 mmol) in N,N-dimethylformamide (1.5 mL) were added N,N-diisopropylethylamine (56 mg, 0.4 mmol) and 4-(7-fluoroimidazo [1,2-a]pyridin-3-yl)-7-[[6-methyl-5-(4-piperidyl)-2-pyridyl]amino]isoindolin-1-one (100 mg, 0.14 mmol, 2 trifluoroacetate), the mixture was stirred at 25° C. for 10 min, followed by the addition of sodium triacetoxyborohydride (92 mg, 0.4 mmol). The reaction mixture was stirred at 50° C. for 10 h, then filtered and concentrated under reduced pressure. The residue was purified by prep-TLC (dichloromethane:methanol=10:1), the crude product was further purified by prep-HPLC (1%-30% acetonitrile in water (formic acid) over 10 min) to afford the title compound (19.3 mg, 16%) as a pale yellow solid. MS (ESI) m/z: 816.6 [M+H]⁺; ¹H NMR (400 MHz, DMSO-d₆) δ 10.99 (s, 1H), 9.99 (s, 1H), 8.81 (s, 1H), 8.73 (d, J=8.4 Hz, 1H), 8.45 (dd, J=6.0, 7.2 Hz, 1H), 7.82 (s, 1H), 7.72 (d, J=8.8 Hz, 1H), 7.65 (d, J=8.4 Hz, 1H), 7.58-7.48 (m, 2H), 7.29 (d, J=8.4 Hz, 1H), 6.97 (td, J=2.4, 7.6 Hz, 1H), 6.83 (d, J=8.4 Hz, 1H), 5.10 (dd, J=4.8, 13.2 Hz, 1H), 4.46-4.35 (m, 3H), 4.31-4.21 (m, 1H), 3.52-3.46 (m, 2H), 3.17-3.06 (m, 2H), 2.97-2.85 (m, 1H), 2.84-2.68 (m, 3H), 2.64-2.55 (m, 2H), 2.52 (s, 3H), 2.47-2.37 (m, 3H), 2.03-1.89 (m, 3H), 1.82-1.63 (m, 6H).

### Exemplification of Method C

### Example 4: Exemplary Synthesis of 3-{5-[4-(1-{6-[(7-{7-fluoroimidazo[1,2-a]pyridin-3-yl}-3-oxo-2,3-dihydro-1H-isoindol-4-yl)amino]-2-methylpyridin-3-yl}piperidin-4-yl)piperazin-1-yl]-3-methyl-2-oxo-2,3-dihydro-1H-1,3-benzodiazol-1-yl}piperidine-2,6-dione

### Step 1: Preparation of 8-(2-methyl-3-pyridyl)-1,4-dioxa-8-azaspiro[4.5]decane

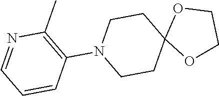 [SMILES: Cc1ncccc1N1CCC2(CC1)OCCO2]

To a solution of 3-bromo-2-methyl-pyridine (10 g, 58.13 mmol, 1 eq), 1,4-dioxa-8-azaspiro[4.5]decane (9.16 g, 63.95 mmol, 8.20 mL, 1.1 eq), cesium carbonate (37.88 g, 116.26 mmol, 2 eq) and [2-(2-aminophenyl)phenyl]-methylsulfonyloxy-palladium;dicyclohexyl-[2-(2,6-diisopropoxyphenyl)phenyl]phosphane (2.43 g, 2.91 mmol, 0.05 eq) in dioxane (100 mL) was degassed and purged with nitrogen for 3 times, and then the mixture was stirred at 100° C. for 12 h under nitrogen atmosphere. The reaction mixture was filtered to remove the insoluble. The solvent was concentrated under vacuum. The residue was purified by column chromatography (Petroleum ether:Ethyl acetate=1:1). The title compound (4.53 g, 19.33 mmol, 33% yield) was obtained as a yellow oil. MS (ESI) m/z: 235.1 [M+H]⁺; ¹H NMR (400 MHz, DMSO-d₆) δ: 8.10 (d, J=3.6 Hz, 1H), 7.38 (d, J=8.0 Hz, 1H), 7.13 (dd, J=4.8, 8.0 Hz, 1H), 3.91 (s, 4H), 2.95-2.86 (m, 4H), 2.43 (s, 3H), 1.82-1.72 (m, 4H)

### Step 2: Preparation of 8-(6-bromo-2-methyl-3-pyridyl)-1,4-dioxa-8-azaspiro[4.5]decane

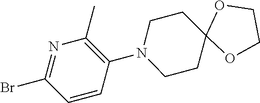 [SMILES: Cc1nc(Br)ccc1N1CCC2(CC1)OCCO2]

To a solution of 8-(2-methyl-3-pyridyl)-1,4-dioxa-8-azaspiro[4.5]decane (4.03 g, 17.20 mmol, 1 eq) in acetonitrile (40 mL) was added 1-bromopyrrolidine-2,5-dione (3.67 g, 20.64 mmol, 1.2 eq) at 0° C. The mixture was stirred at 25° C. for 12 h. The reaction mixture was diluted with water 50 mL and extracted with ethyl acetate (50 mL×3). The combined organic phase was washed with brine (50 mL), dried with anhydrous sodium sulfate, filtered and concentrated in vacuum. The residue was purified by column chromatography (Petroleum ether:Ethyl acetate=3:1). The title compound (2.3 g, 7.34 mmol, 43% yield) was obtained as a yellow oil. MS (ESI) m/z: 315.1 [M+2]; ¹H NMR (400 MHz, DMSO-d₆) δ: 7.41-7.32 (m, 2H), 3.91 (s, 4H), 2.94-2.86 (m, 4H), 2.40 (s, 3H), 1.81-1.72 (m, 4H).

### Step 3. Preparation of tert-butyl 7-[[5-(1,4-dioxa-8-azaspiro[4.5]decan-8-yl)-6-methyl-2-pyridyl]amino]-4-(7-fluoroimidazo[1,2-a]pyridin-3-yl)-1-oxo-isoindoline-2-carboxylate

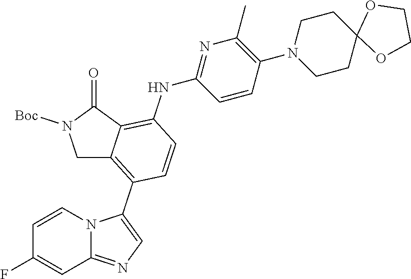 [SMILES: Cc1nc(Nc2ccc(-c3cnc4cc(F)ccn34)c3c2C(=O)N(C(=O)OC(C)(C)C)C3)ccc1N1CCC2(CC1)OCCO2]

To a solution of tert-butyl 7-amino-4-(7-fluoroimidazo[1,2-a]pyridin-3-yl)-1-oxo-isoindoline-2-carboxylate (427 mg, 1.12 mmol, 0.7 eq) 8-(6-bromo-2-methyl-3-pyridyl)-1,4-dioxa-8-azaspiro[4.5]decane (500 mg, 1.60 mmol, 1 eq), tripotassium phosphate (1.02 g, 4.79 mmol, 3 eq), (1E,4E)-1,5-diphenylpenta-1,4-dien-3-one; palladium (146 mg, 0.16 mmol, 0.1 eq) and (5-diphenylphosphanyl-9,9-dimethyl-xanthen-4-yl)-diphenyl-phosphane (92 mg, 0.16 mmol, 0.1 eq) in dioxane (8 mL) was degassed and purged with nitrogen for 3 times, and then the mixture was stirred at 100° C. for 4 h under nitrogen atmosphere. LCMS showed reactant 1 was consumed completely and desired mass was detected. The reaction mixture was diluted with water 20 mL and extracted with ethyl acetate (20 mL×3). The combined organic phase was washed with brine (20 mL), dried with anhydrous sodium sulfate, filtered and concentrated in vacuum. The residue was purified by prep-HPLC (column: Phenomenex luna C18 150*40 mm*15 um; mobile phase: [water(FA)-ACN]; gradient: 25%-55% B over 15 min). The title compound (150 mg, 0.24 mmol, 15.29% yield) was obtained as a yellow solid. MS (ESI) m/z: 615.2 [M+H]

### Step 4: Preparation of 4-(7-fluoroimidazo[1,2-a]pyridin-3-yl)-7-[[6-methyl-5-(4-oxo-1-piperidyl)-2-pyridyl]amino]isoindolin-1-one

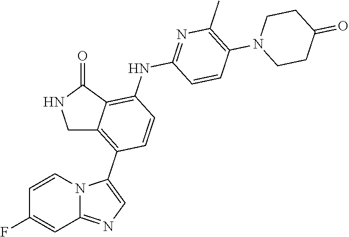 [SMILES: Cc1nc(Nc2ccc(-c3cnc4cc(F)ccn34)c3c2C(=O)NC3)ccc1N1CCC(=O)CC1]

To a solution of tert-butyl 7-[[5-(1,4-dioxa-8-azaspiro[4.5]decan-8-yl)-6-methyl-2-pyridyl]amino]-4-(7-fluoroimidazo[1,2-a]pyridin-3-yl)-1-oxo-isoindoline-2-carboxylate (300 mg, 0.49 mmol, 1 eq) in tetrahydrofuran (3 mL) was added sulfuric acid (2 M, 3 mL, 12.29 eq). The mixture was stirred at 50° C. for 2 h. The reaction mixture was diluted with a saturated aqueous solution of sodium bicarbonate (20 mL) and the pH was adjusted to 7 and extracted with ethyl acetate (20 mL×3). The combined organic phase was washed with brine (20 mL), dried with anhydrous sodium sulfate, filtered and concentrated in vacuum. The residue was purified by prep-HPLC (column: Phenomenex luna C18 150*40 mm*15 um; mobile phase: [water(FA)-ACN]; gradient: 10%-40% B over 15 min). The title compound (200 mg, 0.43 mmol, 87% yield) was obtained as a yellow solid. MS (ESI) m/z: 471.2 [M+H]⁺; ¹H NMR (400 MHz, DMSO-d₆) δ: 9.97 (s, 1H), 8.80 (s, 1H), 8.69 (d, J=8.4 Hz, 1H), 8.49-8.43 (m, 1H), 7.85 (s, 1H), 7.72 (d, J=8.4 Hz, 1H), 7.57-7.47 (m, 2H), 7.02-6.96 (m, 1H), 6.85 (d, J=8.4 Hz, 1H), 4.38 (s, 2H), 3.14 (br t, J=5.6 Hz, 4H), 2.55 (s, 3H), 2.55-2.53 (m, 4H).

### Step 5: Preparation of tert-butyl 4-[1-(2,6-dioxo-3-piperidyl)-3-methyl-2-oxo-benzimidazol-5-yl]piperazine-1-carboxylate

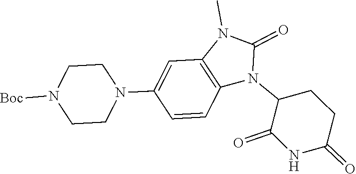 [SMILES: Cn1c(=O)n(C2CCC(=O)NC2=O)c2ccc(N3CCN(C(=O)OC(C)(C)C)CC3)cc21]

A mixture of 3-(5-bromo-3-methyl-2-oxo-benzimidazol-1-yl)piperidine-2,6-dione (1 g, 2.96 mmol, 1 eq) in toluene (30 mL) was added tert-butyl piperazine-1-carboxylate (605 mg, 3.25 mmol, 1.1 eq), 2-dicyclohexylphosphino-2′,6′-diisopropoxybiphenyl (69 mg, 0.14 mmol, 0.05 eq) and chloro(2-dicyclohexylphosphino-2′,6′-diisopropoxy-1,1′-biphenyl)[2-(2′-amino-1,1′-biphenyl)]palladium(II) (229 mg, 0.29 mmol, 0.1 eq) was added to the mixture. The mixture was degassed and purged with nitrogen for 3 times. Then hexamethyldisiliconyl amine lithium (1 M, 14.8 mL, 5 eq) was added to the mixture. The reaction was stirred at 100° C. for 2 h under nitrogen atmosphere. The reaction mixture was quenched by addition formic acid till pH=6, and then diluted with aqueous sodium hydrogen carbonate 60 mL and extracted with dichloromethane (150 mL×3). The combined organic layers were dried over anhydrous sodium sulfate, filtered and concentrated under reduced pressure to give a residue. The crude product was triturated with ethyl acetate for 30 min. The title compound (640 mg, 1.44 mmol, 48% yield) was obtained as a purple solid. MS (ESI) m/z: 444.1 [M+H]⁺; ¹HNMR (400 MHz, DMSO-d₆) δ: 11.06 (s, 1H), 6.96 (d, J=8.8 Hz, 1H), 6.88 (d, J=2.0 Hz, 1H), 6.66 (dd, J=2.0, 8.8 Hz, 1H), 5.30 (dd, J=5.6, 12.8 Hz, 1H), 3.51-3.44 (m, 4H), 3.30 (s, 3H), 3.07-2.98 (m, 4H), 2.94-2.84 (m, 1H), 2.73-2.65 (m, 1H), 2.64-2.58 (m, 1H), 2.01-1.95 (m, 1H), 1.42 (s, 9H)

### Step 6: Preparation of 3-(3-methyl-2-oxo-5-piperazin-1-yl-benzimidazol-1-yl)piperidine-2,6-dione

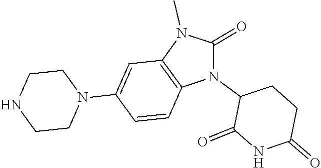 [SMILES: Cn1c(=O)n(C2CCC(=O)NC2=O)c2ccc(N3CCNCC3)cc21]

To a solution of tert-butyl 4-[1-(2,6-dioxo-3-piperidyl)-3-methyl-2-oxo-benzimidazol-5-yl]piperazine-1-carboxylate (200 mg, 0.45 mmol, 1 eq) in dichloromethane (3 mL) was added trifluoroacetic acid (1.54 g, 13.46 mmol, 1.0 mL, 29.85 eq). The mixture was stirred at 20° C. for 1 h. LC-MS showed Reactant 1 was consumed completely. The reaction was concentrated in reduced pressure at 45° C. The residue was used into the next step without further purification. The title compound (206 mg, crude, Trifluoroacetate) was obtained as a purple solid. MS (ESI) m/z: 344.0 [M+1]⁺.

### Step 7: Preparation of 3-[5-[4-[1-[6-[[7-(7-fluoroimidazo[1,2-a]pyridin-3-yl)-3-oxo-isoindolin-4-yl]amino]-2-methyl-3-pyridyl]-4-piperidyl]piperazin-1-yl]-3-methyl-2-oxo-benzimidazol-1-yl]piperidine-2,6-dione

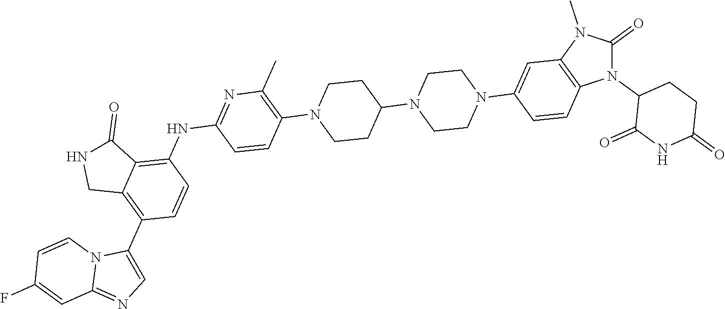 [SMILES: Cc1nc(Nc2ccc(-c3cnc4cc(F)ccn34)c3c2C(=O)NC3)ccc1N1CCC(N2CCN(c3ccc4c(c3)n(C)c(=O)n4C3CCC(=O)NC3=O)CC2)CC1]

To a mixture of 3-(3-methyl-2-oxo-5-piperazin-1-yl-benzimidazol-1-yl)piperidine-2,6-dione (300 mg, 0.65 mmol, 1.5 eq, trifluoroacetate) and 4-(7-fluoroimidazo[1,2-a]pyridin-3-yl)-7-[[6-methyl-5-(4-oxo-1-piperidyl)-2-pyridyl]amino]isoindolin-1-one (206 mg, 0.44 mmol, 1 eq) in dimethylsulfoxide (5 mL) was added 4-methylmorpholine (89 mg, 0.87 mmol, 2 eq). The mixture was stirred at 25° C. for 1 h. Sodium triacetoxyborohydride (185 mg, 0.87 mmol, 2 eq) was added in the mixture. The mixture was stirred at 50° C. for 1 h. The residue was diluted with the solvent (dichloromethane/methanol, v/v=10/1) 10 mL, then sodium bicarbonate was added to adjust pH-8. The mixture was extracted with the solvent (dichloromethane/methanol, v/v=10/1, 10 mL×2). The combined organic layers were washed with water (10 mL×5), dried over sodium sulfate, filtered and concentrated under reduced pressure to give a residue. The residue was purified by prep-HPLC (FA condition; column: Phenomenex luna C18 150*40 mm*15 um; mobile phase: [water(FA)-ACN]; gradient: 2%-32% B over 22 min). The title compound (118.8 mg, 0.15 mmol, 33% yield, 97% purity, formate) was obtained as a white solid. MS (ESI) m/z: 798.6 [M+H]. ¹H NMR (400 MHz, DMSO-d₆) δ: 11.08 (s, 1H), 9.96 (s, 1H), 8.80 (s, 1H), 8.68 (d, J=8.4 Hz, 1H), 8.44 (dd, J=6.0, 7.2 Hz, 1H), 8.16 (s, 1H), 7.80 (s, 1H), 7.72 (d, J=8.4 Hz, 1H), 7.52 (dd, J=2.8, 10.0 Hz, 1H), 7.44 (d, J=8.8 Hz, 1H), 7.00-6.92 (m, 2H), 6.88-6.80 (m, 2H), 6.68-6.60 (m, 1H), 5.32 (dd, J=5.2, 12.8 Hz, 1H), 4.40 (s, 2H), 3.32 (s, 3H), 3.16-3.06 (m, 6H), 2.96-2.84 (m, 1H), 2.76-2.56 (m, 8H), 2.48 (s, 3H), 2.40-2.32 (m, 1H), 2.00-1.88 (m, 3H), 1.68-1.56 (m, 2H).

### Exemplification of Method D

### Example 295: Exemplary Synthesis of 3-(5-(4-(1-(4-(6-((7-(7-fluoroimidazo[1,2-a]pyridin-3-yl)-3-oxoisoindolin-4-yl)amino)-2-methylpyridin-3-yl)piperazin-1-yl)cyclopropyl)piperidin-1-yl)-3-isopropyl-2-oxo-2,3-dihydro-1H-benzo[d]imidazol-1-yl)piperidine-2,6-dione

### Step 1: Preparation of tert-butyl 4-(1-((benzyloxy)carbonyl)piperidine-4-carbonyl)piperazine-1-carboxylate

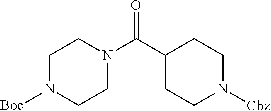 [SMILES: CC(C)(C)OC(=O)N1CCN(C(=O)C2CCN(C(=O)OCc3ccccc3)CC2)CC1]

To a solution of tert-butyl piperazine-1-carboxylate (35.37 g, 189.91 mmol, 1 eq) in N,N-dimethylformamide (300 mL) was added N,N-diisopropylethylamine (73.63 g, 569.72 mmol, 99.2 mL, 3 eq) and 1-benzyloxycarbonylpiperidine-4-carboxylic acid (50 g, 189.91 mmol, 1 eq). Then o-(7-azabenzotriazol-1-yl)-n,n,n′,n′-tetramethyluronium hexafluorophosphate (86.65 g, 227.89 mmol, 1.2 eq) was added to the mixture at 0° C. The mixture was stirred at 20° C. for 1 h. LC-MS showed Reactant 1 was consumed completely. Water (180 mL) was added and the resulting mixture was extracted with Ethyl acetate (3×500 mL). The combined organic layers were washed with brine (4×300 mL), dried over sodium sulfate, filtered, and concentrated. The residue was purified by flash silica gel chromatography (ISCO®; 40 g SepaFlash® Silica Flash Column, Eluent of 65-100% Ethyl acetate/Petroleum ethergradient). The crude product was triturated with tert-butyl methyl ether (200 mL) at 20° C. for 60 min. The crude product was triturated with acetonitrile (100 mL) at 20° C. for 30 min. Compound tert-butyl 4-(1-benzyloxycarbonylpiperidine-4-carbonyl)piperazine-1-carboxylate (60 g, 139.04 mmol, 73% yield) was obtained as a white solid. MS (ESI) m/z: 432.2 [M+1]⁺; ¹HNMR (400 MHz, DMSO-d₆) δ 7.43-7.27 (m, 5H), 5.07 (s, 2H), 4.00 (d, J=13.2 Hz, 2H), 3.49 (s, 2H), 3.40 (d, J=6.4 Hz, 2H), 3.33 (d, J=6.8 Hz, 2H), 3.27 (d, J=2.8 Hz, 2H), 2.96-2.80 (m, 3H), 1.62 (d, J=12.0 Hz, 2H), 1.47-1.36 (m, 11H).

### Step 2: Preparation of tert-butyl 4-(1-(1-((benzyloxy)carbonyl)piperidin-4-yl)cyclopropyl)piperazine-1-carboxylate

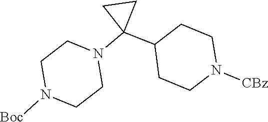

To a solution of ethylmagnesium bromide (3 M, 57.9 mL, 2.5 eq) in tetrahydrofuran (300 mL) was added Titanium(IV) isopropoxide (19.76 g, 69.52 mmol, 20.5 mL, 1 eq) in tetrahydrofuran (200 mL) drop-wise at −70° C. under nitrogen, and the mixture was stirred at −70° C. for 0.5 h. Then tert-butyl 4-(1-benzyloxycarbonylpiperidine-4-carbonyl)piperazine-1-carboxylate (30 g, 69.52 mmol, 1 eq) in nitrogen (300 mL) was added to the mixture drop-wise at −70° C. under nitrogen. The mixture was stirred at 25° C. for another 2 h. TLC (Petroleum ether/Ethyl acetate=3/1) indicated Reactant 1 was consumed completely. Citric acid (1 M, 100 mL) was added to the mixture at 0° C. After filtration, the resulting mixture was extracted with ethyl acetate (3×500 mL). The combined organic layers were dried over sodium sulfate, filtered, and concentrated. The reaction mixture was adjusted pH by addition saturated sodium bicarbonate solution till pH=7, and then diluted with water 200 mL and extracted with dichloromethane (3×500 mL). The combined organic layers were dried over sodium sulfate, filtered and concentrated under reduced pressure to give a residue. The residue was purified by flash silica gel chromatography (ISCO®; 120 g SepaFlash® Silica Flash Column, Eluent of 10-20% Ethyl acetate/Petroleum ethergradient). Compound tert-butyl 4-[1-(1-benzyloxycarbonyl-4-piperidyl)cyclopropyl]piperazine-1-carboxylate (9.15 g, 20.63 mmol, 29% yield) was obtained as a colorless oil. MS (ESI) m/z: 444.3[M+1]⁺; ¹HNMR (400 MHz, DMSO-d₆) δ 7.42-7.26 (m, 5H), 5.05 (s, 2H), 4.09-3.95 (m, 3H), 3.20 (s, 4H), 2.79-2.62 (m, 2H), 2.39 (s, 4H), 1.54 (d, J=10.8 Hz, 2H), 1.38 (s, 9H), 1.28-1.19 (m, 2H), 0.61-0.53 (m, 2H), 0.43-0.30 (m, 2H).

### Step 3: Preparation of tert-butyl 4-(1-(piperidin-4-yl)cyclopropyl)piperazine-1-carboxylate

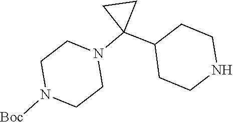 [SMILES: CC(C)(C)OC(=O)N1CCN(C2(C3CCNCC3)CC2)CC1]

To a solution of tert-butyl 4-[1-(1-benzyloxycarbonyl-4-piperidyl)cyclopropyl]piperazine-1-carboxylate (5 g, 11.27 mmol, 1 eq) in tetrahydrofuran (75 mL) was added Palladium on carbon (0.5 g, 10% purity) under nitrogen. The suspension was degassed under vacuum and purged with hydrogen several times. The mixture was stirred under hydrogen (30 psi) at 20° C. for 12 h. LC-MS showed Reactant 1 was consumed completely. The reaction mixture was filtered and the filter was concentrated. The residue was used into the next step without further purification. Compound tert-butyl 4-[1-(4-piperidyl)cyclopropyl]piperazine-1-carboxylate (1.3 g, 4.20 mmol, 37% yield) was obtained as a gray oil. MS (ESI) m/z: 310.2[M+1]⁺; ¹HNMR (400 MHz, DMSO-d₆) δ 3.21 (s, 4H), 2.89 (d, J=11.6 Hz, 2H), 2.48-2.32 (m, 6H), 1.48-1.40 (m, 3H), 1.37 (s, 9H), 1.24-1.10 (m, 3H), 0.59-0.51 (m, 2H), 0.42-0.33 (m, 2H).

### Step 4: Preparation of tert-butyl 4-(1-(1-(1-(2,6-dioxopiperidin-3-yl)-3-isopropyl-2-oxo-2,3-dihydro-1H-benzo[d]imidazol-5-yl)piperidin-4-yl)cyclopropyl)piperazine-1-carboxylate

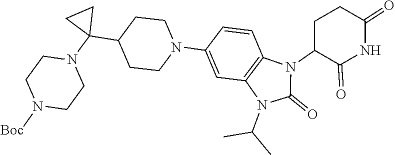 [SMILES: CC(C)n1c(=O)n(C2CCC(=O)NC2=O)c2ccc(N3CCC(C4(N5CCN(C(=O)OC(C)(C)C)CC5)CC4)CC3)cc21]

A mixture of 3-(5-bromo-3-isopropyl-2-oxo-benzimidazol-1-yl)piperidine-2,6-dione (1.40 g, 3.82 mmol, 1 eq) in toluene (20 mL) was added tert-butyl 4-[1-(4-piperidyl)cyclopropyl]piperazine-1-carboxylate (1.3 g, 4.20 mmol, 1.1 eq), Dicyclohexyl(2′,6′-diisopropoxy-[1,1′-biphenyl]-2-yl)phosphine (89 mg, 0.19 mmol, 0.05 eq) and Chloro(2-dicyclohexylphosphino-2′,6′-diisopropoxy-1,1′-biphenyl)[2-(2′-amino-1,1′-biphenyl)]palladium(ii) (296 mg, 0.38 mmol, 0.1 eq) was added to the mixture. The mixture was degassed and purged with nitrogen for 3 times. Then lithium hexamethyldisilazane (1 M, 19.1 mL, 5 eq) was added to the mixture. The reaction was stirred at 100° C. for 2 h under nitrogen atmosphere. TLC (Dichloromethane/Methanol=10/1) indicated Reactant 1 was consumed completely. The reaction mixture was quenched by addition formic acid till pH=6, and then diluted with saturated sodium bicarbonate solution 100 mL and extracted with dichloromethane (200 mL×3). The combined organic layers were dried over sodium sulfate, filtered and concentrated under reduced pressure to give a residue. The residue was purified by flash silica gel chromatography (ISCO®; 40 g SepaFlash® Silica Flash Column, Eluent of 80-100% Ethyl acetate/Petroleum ethergradient). Compound tert-butyl 4-[1-[1-[1-(2,6-dioxo-3-piperidyl)-3-isopropyl-2-oxo-benzimidazol-5-yl]-4-piperidyl]cyclopropyl]piperazine-1-carboxylate (887 mg, 1.49 mmol, 39% yield) was obtained as a yellow foam. MS (ESI) m/z: 595.5 [M+1]⁺; ¹HNMR (400 MHz, DMSO-d₆) δ 11.04 (s, 1H), 6.97-6.85 (m, 2H), 6.66-6.57 (m, 1H), 5.25 (dd, J=5.2, 12.8 Hz, 1H), 4.57 (m, 1H), 3.62-3.47 (m, 4H), 3.29-3.17 (m, 4H), 2.93-2.82 (m, 1H), 2.69-2.55 (m, 3H), 2.46 (s, 4H), 1.96 (d, J=5.2 Hz, 1H), 1.65 (d, J=12.8 Hz, 2H), 1.43 (d, J=6.8 Hz, 6H), 1.38 (s, 9H), 1.30-1.19 (m, 2H), 0.64-0.57 (m, 2H), 0.47-0.41 (m, 2H).

### Step 5: Preparation of 3-(3-isopropyl-2-oxo-5-(4-(1-(piperazin-1-yl)cyclopropyl)piperidin-1-yl)-2,3-dihydro-1H-benzo[d]imidazol-1-yl)piperidine-2,6-dione

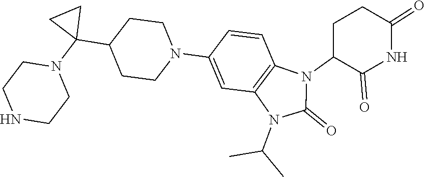 [SMILES: CC(C)n1c(=O)n(C2CCC(=O)NC2=O)c2ccc(N3CCC(C4(N5CCNCC5)CC4)CC3)cc21]

To a solution of tert-butyl 4-[1-[1-[1-(2,6-dioxo-3-piperidyl)-3-isopropyl-2-oxo-benzimidazol-5-yl]-4-piperidyl]cyclopropyl]piperazine-1-carboxylate (887 mg, 1.49 mmol, 1 eq) in dichloromethane (9 mL) was added trifluoroacetic acid (4.61 g, 40.39 mmol, 3 mL, 27.08 eq). The mixture was stirred at 20° C. for 0.5 h. LC-MS showed Reactant 1 was consumed completely. The reaction system was slowly dried with a weak nitrogen stream at room temperature. The residue was used into the next step without further purification. Compound 3-[3-isopropyl-2-oxo-5-[4-(1-piperazin-1-ylcyclopropyl)-1-piperidyl]benzimidazol-1-yl]piperidine-2,6-dione (907 mg, crude, trifluoroacetate) was obtained as a brown oil. MS (ESI) m/z: 495.2[M+1]⁺.

### Step 6: Preparation of 3-(3-isopropyl-5-(4-(1-(4-(2-methyl-4-nitrophenyl)piperazin-1-yl)cyclopropyl)piperidin-1-yl)-2-oxo-2,3-dihydro-1H-benzo[d]imidazol-1-yl)piperidine-2,6-dione

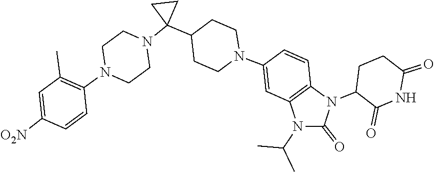 [SMILES: Cc1cc([N+](=O)[O-])ccc1N1CCN(C2(C3CCN(c4ccc5c(c4)n(C(C)C)c(=O)n5C4CCC(=O)NC4=O)CC3)CC2)CC1]

To a solution of 3-[3-isopropyl-2-oxo-5-[4-(1-piperazin-1-ylcyclopropyl)-1-piperidyl]benzimidazol-1-yl]piperidine-2,6-dione (907 mg, 1.49 mmol, 1 eq, trifluoroacetate) in N,N-dimethylformamide (10 mL) was added potassium carbonate (617 mg, 4.47 mmol, 3 eq) and 3-fluoro-2-methyl-6-nitro-pyridine (348 mg, 2.24 mmol, 1.5 eq). The mixture was stirred at 40° C. for 12 h. LC-MS showed Reactant 1 was consumed completely. Water (50 mL) was added and the resulting mixture was extracted with Ethyl acetate (3×100 mL). The combined organic layers were washed with brine (3×100 mL), dried over sodium sulfate, filtered, and concentrated. The residue was purified flash silica gel chromatography (ISCO®; 40 g SepaFlash® Silica Flash Column, Eluent of 80˜100% Ethyl acetate/Petroleum ethergradient). Compound 3-[3-isopropyl-5-[4-[1-[4-(2-methyl-6-nitro-3-pyridyl)piperazin-1-yl]cyclopropyl]-1-piperidyl]-2-oxo-benzimidazol-1-yl]piperidine-2,6-dione (774 mg, 1.23 mmol, 82% yield) was obtained as a brown oil. MS (ESI) m/z: 631.4[M+1]⁺; ¹HNMR (400 MHz, DMSO-d₆) δ 11.05 (s, 1H), 8.11 (d, J=8.8 Hz, 1H), 7.58 (d, J=8.8 Hz, 1H), 6.97-6.87 (m, 2H), 6.62 (dd, J=2.0, 8.4 Hz, 1H), 5.26 (dd, J=5.2, 12.8 Hz, 1H), 4.64-4.52 (m, 1H), 3.57 (d, J=11.6 Hz, 2H), 3.32-3.32 (m, 2H), 2.99 (s, 4H), 2.89 (s, 2H), 2.73 (s, 6H), 2.64-2.60 (m, 1H), 2.60-2.55 (m, 2H), 1.71 (d, J=11.2 Hz, 2H), 1.59-1.49 (m, 2H), 1.44 (d, J=6.8 Hz, 6H), 1.32-1.22 (m, 1H), 0.67 (s, 2H), 0.49 (s, 2H).

### Step 7: Preparation of 3-(5-(4-(1-(4-(6-amino-2-methylpyridin-3-yl)piperazin-1-yl)cyclopropyl)piperidin-1-yl)-3-isopropyl-2-oxo-2,3-dihydro-1H-benzo[d]imidazol-1-yl)piperidine-2,6-dione

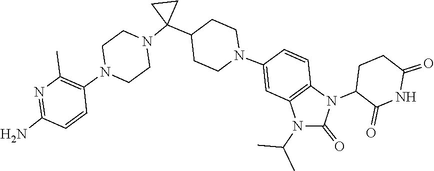 [SMILES: Cc1nc(N)ccc1N1CCN(C2(C3CCN(c4ccc5c(c4)n(C(C)C)c(=O)n5C4CCC(=O)NC4=O)CC3)CC2)CC1]

To a solution of 3-[3-isopropyl-5-[4-[1-[4-(2-methyl-6-nitro-3-pyridyl)piperazin-1-yl]cyclopropyl]-1-piperidyl]-2-oxo-benzimidazol-1-yl]piperidine-2,6-dione (574 mg, 0.91 mmol, 1 eq) in N,N-dimethylformamide (11 mL) was added hypoboric acid (244 mg, 2.73 mmol, 3 eq) and 4-(4-pyridyl)pyridine (6 mg, 0.03 mmol, 0.04 eq). The mixture was stirred at 20° C. for 10 min. LC-MS showed Reactant 1 was consumed completely. The residue was poured into water (50 mL) and stirred for 10 min. The aqueous phase was extracted with ethyl acetate (150 mL×3). The combined organic phase was washed with brine (200 mL×3), dried with anhydrous sodium sulfate, filtered and concentrated in vacuum. The residue was purified by prep-HPLC (column: Phenomenex luna C18 150*40 mm*15 um; mobile phase: [H₂O (0.225% FA)-ACN]; gradient: 1%-25% B over 15.0 min). Compound 3-[5-[4-[1-[4-(6-amino-2-methyl-3-pyridyl)piperazin-1-yl]cyclopropyl]-1-piperidyl]-3-isopropyl-2-oxo-benzimidazol-1-yl]piperidine-2,6-dione (300 mg, 0.46 mmol, 50% yield, formate) was obtained as a brown foam.

MS (ESI) m/z: 601.3[M+1]⁺; ¹HNMR (400 MHz, DMSO-d₆) δ 11.05 (s, 1H), 8.15 (s, 2H), 7.21 (d, J=8.8 Hz, 1H), 6.95-6.85 (m, 2H), 6.62 (dd, J=1.6, 8.8 Hz, 1H), 6.26 (d, J=8.8 Hz, 1H), 6.01-5.41 (m, 2H), 5.26 (dd, J=5.2, 12.8 Hz, 1H), 4.58 (m, 1H), 3.56 (d, J=11.2 Hz, 2H), 2.95-2.78 (m, 1H), 2.64 (d, J=4.4 Hz, 8H), 2.57 (d, J=10.4 Hz, 2H), 2.53 (s, 1H), 2.22 (s, 3H), 2.01-1.92 (m, 1H), 1.69 (d, J=11.2 Hz, 2H), 1.59-1.49 (m, 2H), 1.44 (d, J=6.8 Hz, 6H), 1.26 (d, J=10.0 Hz, 1H), 0.64 (s, 2H), 0.45 (s, 2H).

### Step 8: Preparation of tert-butyl 7-((5-(4-(1-(1-(1-(2,6-dioxopiperidin-3-yl)-3-isopropyl-2-oxo-2,3-dihydro-1H-benzo[d]imidazol-5-yl)piperidin-4-yl)cyclopropyl)piperazin-1-yl)-6-methylpyridin-2-yl)amino)-4-(7-fluoroimidazo[1,2-a]pyridin-3-yl)-1-oxoisoindoline-2-carboxylate

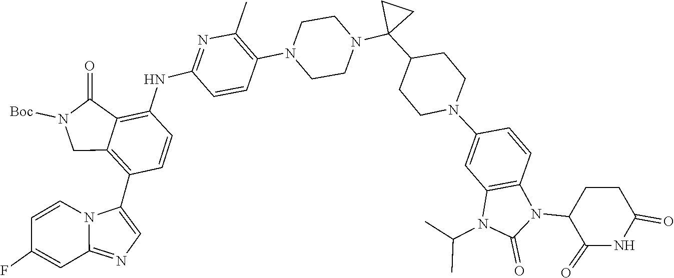 [SMILES: Cc1nc(Nc2ccc(-c3cnc4cc(F)ccn34)c3c2C(=O)N(C(=O)OC(C)(C)C)C3)ccc1N1CCN(C2(C3CCN(c4ccc5c(c4)n(C(C)C)c(=O)n5C4CCC(=O)NC4=O)CC3)CC2)CC1]

A mixture of 3-[5-[4-[1-[4-(6-amino-2-methyl-3-pyridyl)piperazin-1-yl]cyclopropyl]-1-piperidyl]-3-isopropyl-2-oxo-benzimidazol-1-yl]piperidine-2,6-dione (250 mg, 0.38 mmol, 1 eq, formate), tert-butyl 7-bromo-4-(7-fluoroimidazo[1,2-a]pyridin-3-yl)-1-oxo-isoindoline-2-carboxylate (224 mg, 0.50 mmol, 1.3 eq), cesium carbonate (251 mg, 0.77 mmol, 2 eq), methanesulfonato (2-dicyclohexylphosphino-2,6-di-i-propoxy-1, 1-biphenyl) (2-amino-1,1-biphenyl-2-yl) palladium(ii) (32 mg, 0.03 mmol, 0.1 eq) in dioxane (5 mL) was degassed and purged with nitrogen for 3 times, and then the mixture was stirred at 100° C. for 3 h under nitrogen atmosphere. LC-MS showed ˜10% of Reactant 1 remained. The mixture was filtered and the filtrated was concentrated in vacuum. The residue was purified by flash silica gel chromatography (ISCO®; 20 g SepaFlash® Silica Flash Column, Eluent of 80˜100% Ethyl acetate/Petroleum ethergradient). Compound tert-butyl 7-[[5-[4-[1-[1-[1-(2,6-dioxo-3-piperidyl)-3-isopropyl-2-oxo-benzimidazol-5-yl]-4-piperidyl]cyclopropyl]piperazin-1-yl]-6-methyl-2-pyridyl]amino]-4-(7-fluoroimidazo[1,2-a]pyridin-3-yl)-1-oxo-isoindoline-2-carboxylate (225 mg, 0.23 mmol, 60% yield) was obtained as a yellow foam. MS (ESI) m/z: 966.6[M+1]⁺

### Step 9: Preparation of 3-(5-(4-(1-(4-(6-((7-(7-fluoroimidazo[1,2-a]pyridin-3-yl)-3-oxoisoindolin-4-yl)amino)-2-methylpyridin-3-yl)piperazin-1-yl)cyclopropyl)piperidin-1-yl)-3-isopropyl-2-oxo-2,3-dihydro-1H-benzo[d]imidazol-1-yl)piperidine-2,6-dione

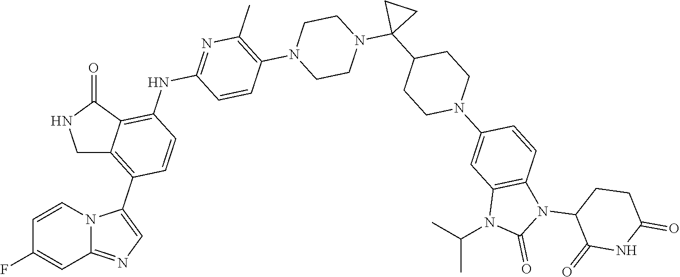 [SMILES: Cc1nc(Nc2ccc(-c3cnc4cc(F)ccn34)c3c2C(=O)NC3)ccc1N1CCN(C2(C3CCN(c4ccc5c(c4)n(C(C)C)c(=O)n5C4CCC(=O)NC4=O)CC3)CC2)CC1]

To a solution of tert-butyl 7-[[5-[4-[1-[1-[1-(2,6-dioxo-3-piperidyl)-3-isopropyl-2-oxo-benzimidazol-5-yl]-4-piperidyl]cyclopropyl]piperazin-1-yl]-6-methyl-2-pyridyl]amino]-4-(7-fluoroimidazo[1,2-a]pyridin-3-yl)-1-oxo-isoindoline-2-carboxylate (220 mg, 0.22 mmol, 1 eq) in dichloromethane (3 mL) was added trifluoroacetic acid (1.54 g, 13.46 mmol, 1 mL, 59.12 eq). The mixture was stirred at 25° C. for 0.5 h. LC-MS showed Reactant 1 was consumed completely. The mixture was diluted with dichloromethane/methanol (V/V=10/1, 50 mL), adjusted to pH˜7 by addition saturated sodium bicarbonate solution, and the resulting mixture was extracted with dichloromethane/methanol (V/V=10/1, 2×50 mL). The combined organic layers were dried over sodium sulfate, filtered, and concentrated. The residue was purified by prep-HPLC (column: Phenomenex Luna C18 150*25 mm*10 um; mobile phase: [H2O (0.225% FA)-ACN]; gradient: 5%-35% B over 10.0 min). Compound 3-[5-[4-[1-[4-[6-[[7-(7-fluoroimidazo[1,2-a]pyridin-3-yl)-3-oxo-isoindolin-4-yl]amino]-2-methyl-3-pyridyl]piperazin-1-yl]cyclopropyl]-1-piperidyl]-3-isopropyl-2-oxo-benzimidazol-1-yl]piperidine-2,6-dione (112.3 mg, 0.11 mmol, 51% yield, 95% purity, formate) was obtained as a yellow solid. MS (ESI) m/z: 866.7[M+1]⁺; ¹HNMR (400 MHz, DMSO-d₆) δ 11.06 (s, 1H), 9.93 (s, 1H), 8.79 (s, 1H), 8.64 (d, J=8.4 Hz, 1H), 8.49-8.38 (m, 1H), 8.16 (s, 1H), 7.81 (s, 1H), 7.70 (d, J=8.4 Hz, 1H), 7.52 (dd, J=2.4, 10.0 Hz, 1H), 7.44 (d, J=8.4 Hz, 1H), 6.99-6.88 (m, 3H), 6.82 (d, J=8.4 Hz, 1H), 6.63 (dd, J=1.2, 8.4 Hz, 1H), 5.27 (dd, J=5.4, 12.4 Hz, 1H), 4.65-4.53 (m, 1H), 4.38 (s, 2H), 3.58 (d, J=10.0 Hz, 2H), 2.93-2.83 (m, 1H), 2.78 (s, 4H), 2.69 (s, 4H), 2.65-2.59 (m, 2H), 2.57 (s, 1H), 2.54 (s, 1H), 2.47-2.43 (m, 3H), 2.01-1.94 (m, 1H), 1.71 (d, J=10.4 Hz, 2H), 1.60-1.50 (m, 2H), 1.45 (d, J=6.8 Hz, 6H), 1.29 (d, J=7.2 Hz, 1H), 0.66 (s, 2H), 0.48 (s, 2H).

### Exemplification of Method E

### Example 296: Exemplary Synthesis of 3-[4-[4-[[4-[[6-[[7-(7-fluoroimidazo[1,2-a]pyridin-3-yl)-3-oxo-isoindolin-4-yl]amino]-2-methyl-3-pyridyl]methoxy]-1-piperidyl]methyl]-1-piperidyl]-3-methyl-2-oxo-benzimidazol-1-yl]piperidine-2,6-dione

### Step 1: Preparation of 6-bromo-3-(bromomethyl)-2-methyl-pyridine

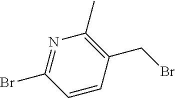

To a solution of (6-bromo-2-methyl-3-pyridyl)methanol (700 mg, 3.5 mmol, 1 eq) in dichloromethane (10 mL) was added n-bromosuccinimide (617 mg, 3.5 mmol, 1 eq) and PPh3 (1 g, 3.8 mmol, 1.1 eq). The mixture was stirred at 25° C. for 1 h. TLC (Petroleum ether:Ethyl acetate=5:1) indicated one spot was detected. The reaction mixture was concentrated under reduced pressure to give a residue. The residue was purified by column chromatography (SiO₂, Petroleum ether/Ethyl acetate=1/0 to 10/1). Compound 6-bromo-3-(bromomethyl)-2-methyl-pyridine (690 mg, 2.6 mmol, 75.17% yield) was obtained as a white solid and used directly in the next reaction step.

### Step 2: Preparation of tert-butyl 4-[(6-bromo-2-methyl-3-pyridyl)methoxy]piperidine-1-carboxylate

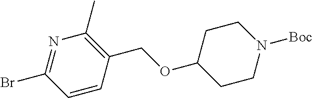 [SMILES: Cc1nc(Br)ccc1COC1CCN(C(=O)OC(C)(C)C)CC1]

To a solution of tert-butyl 4-hydroxypiperidine-1-carboxylate (600 mg, 2.98 mmol, 1 eq) in Dimethylformamide (10 mL) was added sodium hydride (179 mg, 4.5 mmol, 60% purity, 1.5 eq) at 0° C. for 0.5 h, then was added 6-bromo-3-(bromomethyl)-2-methyl-pyridine (790 mg, 3 mmol, 1 eq) to the mixture. The mixture was stirred at 0° C. for 1.5 h. TLC (Petroleum ether:Ethyl acetate=3:1) indicated one spot was detected. The reaction mixture was quenched by addition water (30 mL) at 0° C., and then extracted with ethyl acetate (10 mL*3). concentrated under reduced pressure to give a residue. The residue was purified by column chromatography (SiO₂, Petroleum ether/Ethyl acetate=1/0 to 5/1). Compound tert-butyl 4-[(6-bromo-2-methyl-3-pyridyl)methoxy]piperidine-1-carboxylate (600 mg, 1.6 mmol, 52.23% yield) was obtained as a white solid and used directly in the next reaction step.

### Step 3: Preparation of tert-butyl 7-[[5-[(1-tert-butoxycarbonyl-4-piperidyl)oxymethyl]-6-methyl-2-pyridyl]amino]-4-(7-fluoroimidazo[1,2-a]pyridin-3-yl)-1-oxo-isoindoline-2-carboxylate

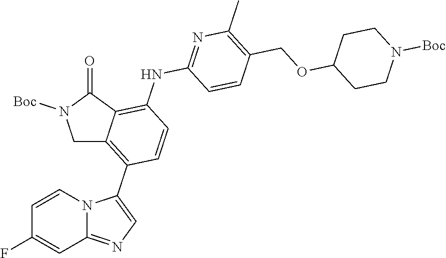 [SMILES: Cc1nc(Nc2ccc(-c3cnc4cc(F)ccn34)c3c2C(=O)N(C(=O)OC(C)(C)C)C3)ccc1COC1CCN(C(=O)OC(C)(C)C)CC1]

A mixture of tert-butyl 4-[(6-bromo-2-methyl-3-pyridyl)methoxy]piperidine-1-carboxylate (574 mg, 1.49 mmol, 1.2 eq), tert-butyl 7-amino-4-(7-fluoroimidazo[1,2-a]pyridin-3-yl)-1-oxo-isoindoline-2-carboxylate (475 mg, 1.24 mmol, 1 eq), potassium phosphate (791 mg, 3.73 mmol, 3 eq), (5-diphenylphosphanyl-9,9-dimethyl-xanthen-4-yl)-diphenylphosphane (72 mg, 0.12 mmol, 0.1 eq) and (1E,4E)-1,5-diphenylpenta-1,4-dien-3-one; palladium (114 mg, 0.12 mmol, 0.1 eq) in dioxane (10 mL) was degassed and purged with nitrogen for 3 times, and then the mixture was stirred at 100° C. for 2 hr under nitrogen atmosphere. LC-MS showed one peak with desired mass was detected. The reaction mixture was concentrated under reduced pressure to give a residue. The residue was purified by column chromatography (SiO₂, Petroleum ether/Ethyl acetate=1/0 to 2/1). Compound tert-butyl 7-[[5-[(1-tert-butoxycarbonyl-4-piperidyl)oxymethyl]-6-methyl-2-pyridyl]amino]-4-(7-fluoroimidazo[1,2-a]pyridin-3-yl)-1-oxo-isoindoline-2-carboxylate (277 mg, 0.4 mmol, 32.47% yield) was obtained as a white solid. MS (ESI) m/z: 687.4 [M+1]⁺; ¹H NMR (400 MHz, CDCl3) δ 9.93 (s, 1H), 9.02 (d, J=8.4 Hz, 1H), 8.07-7.96 (m, 1H), 7.68-7.60 (m, 2H), 7.56 (d, J=8.4 Hz, 1H), 7.37-7.31 (m, 1H), 6.78-6.69 (m, 2H), 4.64 (s, 2H), 4.51 (s, 2H), 3.85-3.68 (m, 3H), 3.65-3.54 (m, 1H), 3.18-3.08 (m, 2H), 2.57 (s, 3H), 1.89 (br s, 2H), 1.59 (s, 9H), 1.47 (s, 9H), 1.27-1.22 (m, 1H).

### Step 4: Preparation of 4-(7-fluoroimidazo[1,2-a]pyridin-3-yl)-7-[[6-methyl-5-(4-piperidyloxymethyl)-2-pyridyl]amino]isoindolin-1-one

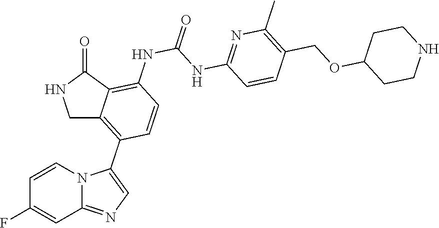 [SMILES: Cc1nc(NC(=O)Nc2ccc(-c3cnc4cc(F)ccn34)c3c2C(=O)NC3)ccc1COC1CCNCC1]

To a solution of tert-butyl 7-[[5-[(1-tert-butoxycarbonyl-4-piperidyl)oxymethyl]-6-methyl-2-pyridyl]amino]-4-(7-fluoroimidazo[1,2-a]pyridin-3-yl)-1-oxo-isoindoline-2-carboxylate (260 mg, 0.38 mmol, 1 eq) in dichloromethane (0.5 mL) was added trifluoroacetic acid (20 g, 175.01 mmol, 13.00 mL, 462.27 eq). The mixture was stirred at 25° C. for 0.5 h. TLC (Dichloromethane:Methanol=10:1) indicated one spot was detected. The reaction mixture was concentrated under reduced pressure to give a residue. The crude product 4-(7-fluoroimidazo[1,2-a]pyridin-3-yl)-7-[[6-methyl-5-(4-piperidyloxymethyl)-2-pyridyl]amino]isoindolin-1-one (265 mg, 370.84 μmol, 97.96% yield, 2 trifluoroacetic acid) was used into the next step without further purification. Compound 4-(7-fluoroimidazo[1,2-a]pyridin-3-yl)-7-[[6-methyl-5-(4-piperidyloxymethyl)-2-pyridyl]amino]isoindolin-1-one (265 mg, 0.37 mmol, 97.96% yield, 2 trifluoroacetic acid) was obtained as a white solid and used directly in the next reaction step.

### Step 5: Preparation of 3-[4-[4-[[4-[[6-[[7-(7-fluoroimidazo[1,2-a]pyridin-3-yl)-3-oxo-isoindolin-4-yl]amino]-2-methyl-3-pyridyl]methoxy]-1-piperidylmethyl-1-piperidyl]-3-methyl-2-oxo-benzimidazol-1-yl]piperidine-2,6-dione

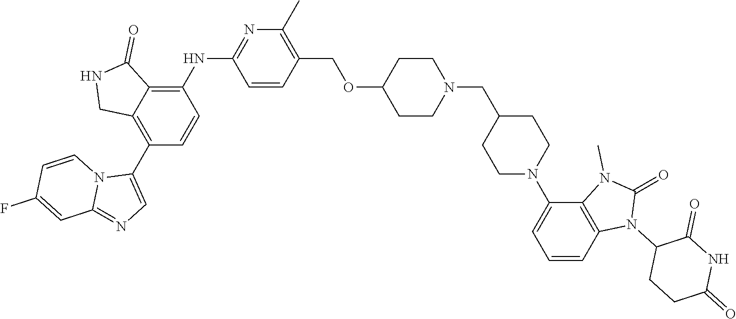 [SMILES: Cc1nc(Nc2ccc(-c3cnc4cc(F)ccn34)c3c2C(=O)NC3)ccc1COC1CCN(CC2CCN(c3cccc4c3n(C)c(=O)n4C3CCC(=O)NC3=O)CC2)CC1]

To a solution of 4-(7-fluoroimidazo[1,2-a]pyridin-3-yl)-7-[[6-methyl-5-(4-piperidyloxymethyl)-2-pyridyl]amino]isoindolin-1-one (80 mg, 0.1 mmol, 1 eq, 2 Triluoroacetic acid) and 1-[1-(2,6-dioxo-3-piperidyl)-3-methyl-2-oxobenzimidazol-4-yl]piperidine-4-carbaldehyde (54 mg, 0.1 mmol, 1 eq, Trifuoroacetic acid) in dichloromethane (1.5 mL) and dimethyl sulfoxide (1.5 mL) was added N-methylmorphoiline (34 mg, 0.3 mmol, 0.04 mL, 3 eq) and sodium triacetoxyborohydride (36 mg, 0.17 mmol, 1.5 eq). The mixture was stirred at 25° C. for 2 h. LC-MS showed one peak with desired mass was detected. The reaction mixture was concentrated under reduced pressure to give a residue. The residue was purified by prep-HPLC (column: YMC-Actus Triart C18 150*30 mm*7 um; mobile phase: [water(FA)-acetonitrile]; gradient: 13%-43% B over 10 min). Compound 3-[4-[4-[[4-[[6-[[7-(7-fluoroimidazo[1,2-a]pyridin-3-yl)-3-oxo-isoindolin-4-yl]amino]-2-methyl-3-pyridyl]methoxy]-1-piperidyl]methyl]-1-piperidyl]-3-methyl-2-oxo-benzimidazol-1-yl]piperidine-2,6-dione (20.1 mg, 0.02 mmol, 21.14% yield, 99% purity) was obtained as a white solid. MS (ESI) m/z: 841.6 [M+1]⁺; ¹H NMR (400 MHz, DMSO-d₆) δ 11.13-11.02 (m, 1H), 10.06 (s, 1H), 8.82 (s, 1H), 8.76 (d, J=8.4 Hz, 1H), 8.48-8.42 (m, 1H), 8.28 (br s, 1H), 7.82 (s, 1H), 7.73 (d, J=8.4 Hz, 1H), 7.60 (d, J=8.4 Hz, 1H), 7.52 (dd, J=2.6, 10.0 Hz, 1H), 7.00-6.93 (m, 2H), 6.91-6.80 (m, 3H), 5.37-5.29 (m, 1H), 4.46 (s, 2H), 4.39 (s, 2H), 3.62 (s, 3H), 3.12-3.07 (m, 2H), 2.93-2.82 (m, 2H), 2.71-2.57 (m, 7H), 2.22-2.15 (m, 2H), 2.11-1.96 (m, 4H), 1.93-1.87 (m, 2H), 1.81 (br d, J=12.0 Hz, 2H), 1.71-1.45 (m, 4H), 1.33-1.23 (m, 2H).

### Exemplification of Method F

### Example 297: Exemplary Synthesis of 3-(4-fluoro-5-{6-[1-(4-{6-[(7-{7-fluoroimidazo[1,2-a]pyridin-3-yl}-3-oxo-2,3-dihydro-1H-isoindol-4-yl)amino]-2-methylpyridin-3-yl}piperidin-1-yl)ethyl]-2-azaspiro[3.3]heptan-2-yl}-1-oxo-2,3-dihydro-1H-isoindol-2-yl)piperidine-2,6-dione

### Step 1: Preparation of 6-methyl-5-(4-piperidyl)pyridin-2-amine

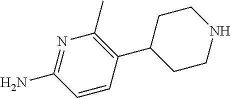

A mixture of tert-butyl 4-(6-amino-2-methyl-3-pyridyl)piperidine-1-carboxylate (5 g, 17.16 mmol, 1 eq) in hydrogen chloride/dioxane (2 M, 50 mL, 5.83 eq) was stirred at 20° C. for 1 h. LCMS showed desired MS was detected. The mixture was concentrated in vacuum. 6-methyl-5-(4-piperidyl)pyridin-2-amine (3.9 g, crude, hydrochloride) was obtained as a white solid. MS (ESI) m/z: 192.2 [M+1]⁺; ¹HNMR (400 MHz, Dimethylsulfoxide-d₆) δ 14.46 (s, 1H), 9.23 (s, 2H), 7.95-7.76 (m, 2H), 7.73 (d, J=9.2 Hz, 1H), 6.90 (d, J=9.2 Hz, 1H), 3.30 (d, J=12.0 Hz, 2H), 3.07-2.89 (m, 3H), 2.47 (s, 3H), 1.97-1.83 (m, 2H), 1.79-1.68 (m, 2H).

### Step 2: Preparation of tert-butyl 4-(6-amino-2-methyl-3-pyridyl)-3,6-dihydro-2H-pyridine-1-carboxylate

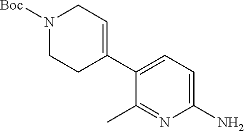 [SMILES: Cc1nc(N)ccc1C1=CCN(C(=O)OC(C)(C)C)CC1]

To a solution of 5-bromo-6-methyl-pyridin-2-amine (5 g, 26.73 mmol, 1 eq), tert-butyl 4-(4,4,5,5-tetramethyl-1,3,2-dioxaborolan-2-yl)-3,6-dihydro-2H-pyridine-1-carboxylate (9.09 g, 29.41 mmol, 1.1 eq), tripotassium phosphate (14.19 g, 66.83 mmol, 2.5 eq) in dioxane (200 mL) and water (20 mL) was degassed and purged with nitrogen for 3 times, and then Chlorine (2-dicyclohexylphosphone-2′,4′,6′-Triisopropyl-1,1′-BIphenyl)(2′-amino-1,1′-biphenyl-2-yl)palladium (II) (1.05 g, 1.34 mmol, 0.05 eq) was added to the mixture under nitrogen. The mixture was stirred at 140° C. for 4 h under nitrogen atmosphere. LC-MS showed Reactant 1 was consumed completely. The mixture was cooled to 20° C. and diluted with brine 50 mL and extracted with dichloromethane 300 mL. The aqueous phase was extracted with dichloromethane (30 mL×3). The combined organic phase was dried with anhydrous sodium sulfate, filtered and concentrated in vacuum. The residue was purified by column chromatography (Petroleum ether/Ethyl acetate=1/0 to 0/1). Compound tert-butyl 4-(6-amino-2-methyl-3-pyridyl)-3,6-dihydro-2H-pyridine-1-carboxylate (6.53 g, 22.57 mmol, 84% yield) was obtained as a brown solid. MS (ESI) m/z: 290.3 [M+1]⁺; ¹H NMR (400 MHz, DMSO-d6) δ 7.08 (d, J=8.4 Hz, 1H), 6.23 (d, J=8.4 Hz, 1H), 5.76 (s, 2H), 5.49 (s, 1H), 3.94 (s, 2H), 3.49 (t, J=5.6 Hz, 2H), 3.17 (d, J=5.2 Hz, 2H), 2.20 (s, 3H), 1.42 (s, 9H).

### Step 3: Preparation of tert-butyl 4-(6-amino-2-methyl-3-pyridyl)piperidine-1-carboxylate

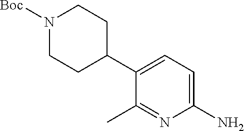 [SMILES: Cc1nc(N)ccc1C1CCN(C(=O)OC(C)(C)C)CC1]

To a solution of tert-butyl 4-(6-amino-2-methyl-3-pyridyl)-3,6-dihydro-2H-pyridine-1-carboxylate (6.5 g, 22.46 mmol, 1 eq) in methanol (70 mL) was added palladium on carbon (1 g, 10% purity) under nitrogen. The mixture was stirred at 25° C. under hydrogen (50 Psi) for 12 h. LCMS showed the desired mass was detected. The mixture was filtered and the filter was concentrated in vacuum. The residue was used into next step without further purification. Compound tert-butyl 4-(6-amino-2-methyl-3-pyridyl)piperidine-1-carboxylate (8.9 g, crude) was obtained as white solid. MS (ESI) m/z: 292.2[M+1]⁺; ¹H NMR (400 MHz, DMSO-d6) δ 7.17 (d, J=8.4 Hz, 1H), 6.24 (d, J=8.4 Hz, 1H), 5.56 (s, 2H), 4.05 (d, J=10.0 Hz, 2H), 2.87-2.65 (m, 3H), 2.27 (s, 3H), 1.60 (d, J=12.8 Hz, 2H), 1.41 (s, 9H), 1.38-1.34 (m, 2H).

### Step 4: Preparation of 4-bromo-7-nitro-isoindolin-1-one

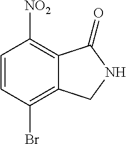 [SMILES: O=C1NCc2c(Br)ccc([N+](=O)[O-])c21]

To a solution of 4-bromoisoindolin-1-one (50 g, 235.80 mmol, 1 eq) in sulfuric acid (736.00 g, 7.50 mol, 400 mL, 31.82 eq) was added nitric acid (43.70 g, 471.60 mmol, 31 mL, 68% purity, 2 eq) at −10° C. The mixture was stirred at 25° C. for 4 h. LCMS indicated reactant 1 was consumed completely and one new spot formed. The reaction mixture was poured into ice water (100 mL). The precipitate that formed was collected by filtration and dried under reduced pressure to give a residue. The crude product was washed with water (200 mL), filtered and concentrated under reduced pressure to give a residue. The crude product was used into the next step without further purification. Compound 4-bromo-7-nitro-isoindolin-1-one (53 g, 206.19 mmol, 87% yield) was obtained as a white solid. MS (ESI) m/z: 258.8 [M+1]⁺; ¹H NMR (400 MHz, DMSO-d₆) δ 9.19 (s, 1H), 8.05 (d, J=8.4 Hz, 1H), 7.87 (d, J=8.4 Hz, 1H), 4.39 (s, 2H).

### Step 5: Preparation of tert-butyl 4-bromo-7-nitro-1-oxo-isoindoline-2-carboxylate

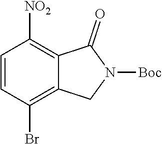 [SMILES: CC(C)(C)OC(=O)N1Cc2c(Br)ccc([N+](=O)[O-])c2C1=O]

To a solution of 4-bromo-7-nitro-isoindolin-1-one (23 g, 89.48 mmol, 1 eq) in dichloromethane (200 mL) was added trimethylamine (27.16 g, 268.44 mmol, 37 mL, 3 eq), dimethylaminopyridine (1.09 g, 8.95 mmol, 0.1 eq) and di-tert-butyl dicarbonate (23.43 g, 107.38 mmol, 25 mL, 1.2 eq). The mixture was stirred at 25° C. for 2 h. LCMS showed reactant 1 was consumed completely and one main peak was detected. The reaction mixture was diluted with water (800 mL) and extracted with ethyl acetate (150 mL×3). The combined organic phase was washed with saturated brine (800 mL), dried with anhydrous sodium sulfate, filtered and concentrated in vacuum. The residue was purified by flash silica gel chromatography (ISCO®; 120 g SepaFlash® Silica Flash Column, Eluent of 15-100% Ethylacetate/Petroleum ethergradient @120 mL/min). Compound tert-butyl 4-bromo-7-nitro-1-oxo-isoindoline-2-carboxylate (29.3 g, 82.04 mmol, 92% yield) was obtained as a yellow solid. MS (ESI) m/z: 300.9 [M−56+1]; ¹H NMR (400 MHz, DMSO-d₆) δ 8.18 (d, J=8.4 Hz, 1H), 7.95 (d, J=8.4 Hz, 1H), 4.73 (s, 2H), 1.52 (s, 9H).

### Step 6: Preparation of tert-butyl 7-amino-4-bromo-1-oxo-isoindoline-2-carboxylate

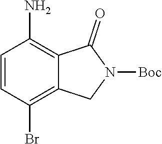 [SMILES: CC(C)(C)OC(=O)N1Cc2c(Br)ccc(N)c2C1=O]

To a solution of tert-butyl 4-bromo-7-nitro-1-oxo-isoindoline-2-carboxylate (24 g, 67.20 mmol, 1 eq) in ethyl alcohol (200 mL) and water (40 mL) was added iron powder (18.76 g, 335.99 mmol, 5 eq) and ammonium chloride (35.94 g, 671.97 mmol, 10 eq) at 60° C. The mixture was stirred at 60° C. for 2 h. LCMS showed reactant 1 was consumed completely and one new peak was detected. The reaction mixture was filtered to remove the insoluble. The residue was purified by flash silica gel chromatography (ISCO®; 80 g SepaFlash® Silica Flash Column, Eluent of 0-30% Ethylacetate/Petroleum ethergradient @120 mL/min). Compound tert-butyl 7-amino-4-bromo-1-oxo-isoindoline-2-carboxylate (10 g, 30.56 mmol, 45% yield) was obtained as a yellow solid. MS (ESI) m/z: 227.1 [M−100+1]⁺; ¹H NMR (400 MHz, DMSO-d₆) δ 7.42 (d, J=8.8 Hz, 1H), 6.61 (d, J=8.8 Hz, 1H), 6.57-6.32 (m, 2H), 4.50 (s, 2H), 1.51 (s, 9H).

### Step 7: Preparation of tert-butyl 7-amino-4-(7-fluoroimidazo[1,2-a]pyridin-3-yl)-1-oxo-isoindoline-2-carboxylate

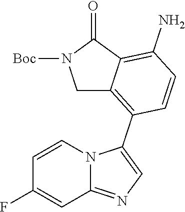 [SMILES: CC(C)(C)OC(=O)N1Cc2c(-c3cnc4cc(F)ccn34)ccc(N)c2C1=O]

A mixture of tert-butyl 7-amino-4-bromo-1-oxo-isoindoline-2-carboxylate (10 g, 30.56 mmol, 1 eq), 7-fluoroimidazo[1,2-a]pyridine (6.24 g, 45.85 mmol, 1.5 eq), Palladium(II) acetate (686 mg, 3.06 mmol, 0.1 eq), potassium acetate (6.00 g, 61.13 mmol, 2 eq) in N,N-dimethylacetamide (100 mL) was degassed and purged with nitrogen for 3 times, and then the mixture was stirred at 110° C. for 12 h under nitrogen atmosphere. LCMS showed reactant 1 was consumed completely and one new peak was detected. The reaction mixture was diluted with water (800 mL) and extracted with ethyl acetate (1200 mL×3). The combined organic phase was washed with saturated brine (600 mL), dried with anhydrous sodium sulfate, filtered and concentrated in vacuum. The crude product was triturated with ethyl acetate at 25° C. for 60 min. The residue was purified by flash silica gel chromatography (ISCO®; 80 g SepaFlash® Silica Flash Column, Eluent of 40-100% Ethylacetate/Petroleum ethergradient @120 mL/min). Compound tert-butyl 7-amino-4-(7-fluoroimidazo[1,2-a]pyridin-3-yl)-1-oxo-isoindoline-2-carboxylate (8.8 g, 23.01 mmol, 75% yield) was obtained as a yellow solid. MS (ESI) m/z: 383.3 [M+1]⁺; ¹H NMR (400 MHz, DMSO-d₆) δ 8.28-8.20 (m, 1H), 7.71 (s, 1H), 7.52-7.45 (m, 2H), 6.95 (dt, J=2.4, 7.6 Hz, 1H), 6.80 (d, J=8.4 Hz, 1H), 6.67 (s, 2H), 4.59 (s, 2H), 1.47 (s, 9H).

### Step 8: Preparation of tert-butyl 7-bromo-4-(7-fluoroimidazo[1,2-a]pyridin-3-yl)-1-oxo-isoindoline-2-carboxylate

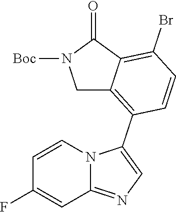 [SMILES: CC(C)(C)OC(=O)N1Cc2c(-c3cnc4cc(F)ccn34)ccc(Br)c2C1=O]

To a solution of tert-butyl 7-amino-4-(7-fluoroimidazo[1,2-a]pyridin-3-yl)-1-oxo-isoindoline-2-carboxylate (500 mg, 1.31 mmol, 1 eq), copper(II) bromide (438 mg, 1.96 mmol, 0.1 mL, 1.5 eq) in acetonitrile (5 mL) was added tert-butyl nitrite (202 mg, 1.96 mmol, 0.2 mL, 1.5 eq) at 25° C. under nitrogen. The reaction mixture was stirred at 25° C. for 1 h. LCMS showed reactant 1 was consumed completely and one new peak was detected. The reaction mixture was diluted with water (100 mL) and extracted with ethyl acetate (30 mL×3). The combined organic phase was washed with saturated brine (100 mL), dried with anhydrous sodium sulfate, filtered and concentrated in vacuum. The residue was purified by prep-TLC (Ethyl acetate:Petroleum ether=3:1). Compound tert-butyl 7-bromo-4-(7-fluoroimidazo[1,2-a]pyridin-3-yl)-1-oxo-isoindoline-2-carboxylate (320 mg, 0.54 mmol, 42% yield, 76% purity) was obtained as a yellow solid. MS (ESI) m/z: 448.0 [M+1]⁺; ¹H NMR (400 MHz, DMSO-d₆) δ 8.43 (dd, J=5.6, 7.2 Hz, 1H), 7.99-7.76 (m, 3H), 7.58 (dd, J=2.4, 10.0 Hz, 1H), 7.09-6.95 (m, 1H), 4.87-4.67 (m, 2H), 1.50 (s, 9H).

### Step 9: Preparation of methyl 4-bromo-2-(bromomethyl)-3-fluorobenzoate

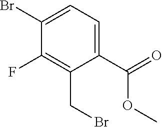

To a solution of methyl 4-bromo-3-fluoro-2-methyl-benzoate (2 g, 8.10 mmol, 1 eq) in CCl₄ (20 mL) was added AIBN (133 mg, 0.81 mmol, 0.1 eq) and NBS (1.44 g, 8.10 mmol, 1 eq). The mixture was stirred at 80° C. for 16 h. LC-MS showed small amount of starting material remained, desired compound was detected as the major product. The reaction mixture was partitioned between water 30 mL and DCM 60 mL (20 mL*3). The organic phase was separated, dried over Na₂SO₄, filtered and concentrated under reduced pressure to give a residue. The residue was purified by column chromatography (SiO₂, Petroleum ether/Ethyl acetate=10/1 to 6/1). Compound methyl 4-bromo-2-(bromomethyl)-3-fluoro-benzoate (2.3 g, 7.06 mmol, 87% yield) was obtained as a yellow solid. ¹H NMR (400 MHz, CDCl₃) (7.71-7.69 (m, 1H), 7.74-7.54 (m, 1H), 5.09-4.89 (m, 2H), 3.98 (s, 3H).

### Step 10: Preparation of 3-(5-bromo-4-fluoro-1-oxoisoindolin-2-yl)piperidine-2,6-dione

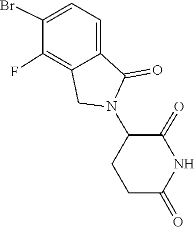 [SMILES: O=C1CCC(N2Cc3c(ccc(Br)c3F)C2=O)C(=O)N1]

To a solution of 3-aminopiperidine-2,6-dione (904 mg, 5.49 mmol, 7.78e−1 eq, HCl) in DMF (25 mL) was added DIEA (4.56 g, 35.28 mmol, 6.15 mL, 5 eq) and stirred for 30 min, then methyl 4-bromo-2-(bromomethyl)-3-fluoro-benzoate (2.3 g, 7.06 mmol, 1 eq) was added. The mixture was stirred at 100° C. for 11.5 h. LC-MS showed no starting material remained, desired compound was detected as the major product. The reaction mixture was poured into cold water (60 mL), the precipitate formed. The mixture was filtered and the filtrate cake was dried in vacuum. The mixture was triturated with PE and methyl-tertbutyl ether (V/V=1:1, 20 mL). Compound 3-(5-bromo-4-fluoro-1-oxo-isoindolin-2-yl)piperidine-2,6-dione (1.4 g, 4.10 mmol, 58% yield) was obtained as a gray solid. MS (ESI) m/z: 341.0 [M+1]⁺; ¹H NMR (400 MHz, DMSO-d₆) δ 11.03 (s, 1H), 7.89 (dd, J=6.0, 8.0 Hz, 1H), 7.56 (d, J=8.0 Hz, 1H), 5.13 (dd, J=5.2, 13.2 Hz, 1H), 4.73-4.05 (m, 2H), 2.90 (s, 1H), 2.44 (br dd, J=4.4, 12.8 Hz, 2H), 2.09-1.90 (m, 1H).

### Step 11: Preparation of tert-butyl 6-[methoxy(methyl)carbamoyl]-2-azaspiro[3.3]heptane-2-carboxylate

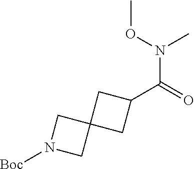 [SMILES: CON(C)C(=O)C1CC2(C1)CN(C(=O)OC(C)(C)C)C2]

To a solution of 2-tert-butoxycarbonyl-2-azaspiro[3.3]heptane-6-carboxylic acid (3 g, 12.43 mmol, 1 eq) in dichloromethane (30 mL) was added triethylamine (3.77 g, 37.30 mmol, 5.2 mL, 3 eq), N-methoxymethanamine (1.82 g, 18.65 mmol, 1.5 eq, hydrochloride) and 2,4,6-tributyl-1,3,5,2,4,6trioxatriphosphinane 2,4,6-trioxide (13.44 g, 18.65 mmol, 50% purity, 1.5 eq) at for 0° C. The mixture was stirred at 25° C. for 12 h. TLC (Petroleum ether:Ethyl acetate=1:1) indicated reactant 1 was consumed completely and one new spot formed. The reaction mixture was diluted with water (100 mL) and extracted with dichloromethane (50 mL×3). The combined organic phase was washed with saturated brine (100 mL), dried with anhydrous sodium sulfate, filtered and concentrated in vacuum. The residue was purified by flash silica gel chromatography (ISCO®; 40 g SepaFlash® Silica Flash Column, Eluent of 30-50% Ethylacetate/Petroleum ethergradient at 120 mL/min). Compound tert-butyl 6-[methoxy(methyl)carbamoyl]-2-azaspiro[3.3]heptane-2-carboxylate (3 g, 10.55 mmol, 84.85% yield) was obtained as a yellow oil.

¹H NMR (400 MHz, DMSO-d6) δ 3.86 (s, 2H), 3.71 (s, 2H), 3.60 (s, 3H), 3.30-3.21 (m, 1H), 3.06 (s, 3H), 2.27 (d, J=8.4 Hz, 4H), 1.35 (s, 9H).

### Step 12: Preparation of tert-butyl 6-acetyl-2-azaspiro[3.3]heptane-2-carboxylate

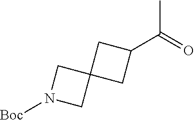 [SMILES: CC(=O)C1CC2(C1)CN(C(=O)OC(C)(C)C)C2]

To a solution of tert-butyl 6-[methoxy(methyl)carbamoyl]-2-azaspiro[3.3]heptane-2-carboxylate (3 g, 10.55 mmol, 1 eq) in tetrahydrofuran (40 mL) was degassed and purged with nitrogen for 3 times. Methyl magnesium bromide (3 M, 7.0 mL, 2 eq) was added the mixture reaction at 0° C. The mixture was stirred at 25° C. for 2 h under nitrogen atmosphere. TLC (Petroleum ether:Ethyl acetate=1:1) showed new spots was detected. The reaction was quenched by saturated ammonium chloride (40 mL) and extracted with ethyl acetate (15 mL×2). The combined organic phase was washed with saturated brine (50 mL), dried with anhydrous sodium sulfate, filtered and concentrated in vacuum. The residue was purified by column chromatography (Petroleum ether:Ethyl acetate=1:1). Compound tert-butyl 6-acetyl-2-azaspiro[3.3]heptane-2-carboxylate (2 g, 8.36 mmol, 79% yield) was obtained as a yellow solid. ¹H NMR (400 MHz, DMSO-d6) δ 3.84 (s, 2H), 3.67 (s, 2H), 3.17-3.08 (m, 1H), 2.30-2.19 (m, 4H), 2.01 (s, 3H), 1.35 (s, 9H).

### Step 13: Preparation of tert-butyl 6-[1-[4-(6-amino-2-methyl-3-pyridyl)-1-piperidyl]ethyl]-2-azaspiro[3.3]heptane-2-carboxylate

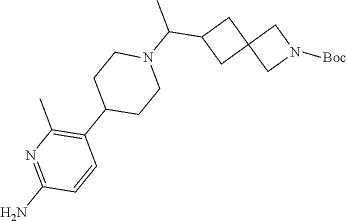 [SMILES: Cc1nc(N)ccc1C1CCN(C(C)C2CC3(C2)CN(C(=O)OC(C)(C)C)C3)CC1]

To a solution 6-methyl-5-(4-piperidyl)pyridin-2-amine (635 mg, 2.79 mmol, 1 eq, hydrochloride) in dichloromethane (10 mL) and dimethyl sulfoxide (10 mL) was added diisopropylethylamine (1.08 g, 8.37 mmol, 1.5 mL, 3 eq) and tert-butyl 6-acetyl-2-azaspiro[3.3]heptane-2-carboxylate (1.00 g, 4.18 mmol, 1.5 eq), the mixture was stirred at 40° C. for 2 h. Tetraisopropoxytitanium (1.58 g, 5.58 mmol, 1.7 mL, 2 eq) and sodium triacetoxyboranuide (1.77 g, 8.37 mmol, 3 eq) was added to the mixture at 20° C. The reaction mixture was stirred at 40° C. for 10 h. LCMS showed reactant 1 was consumed completely and with desired mass was detected. The reaction mixture was filtered to remove the insoluble. The solvent was concentrated under vacuum. The residue was purified by prep-HPLC (column: Phenomenex luna C18 150*40 mm*15 um; mobile phase: [water(FA)-ACN]; gradient: 0%-28% B over 15 min). Compound tert-butyl 6-[1-[4-(6-amino-2-methyl-3-pyridyl)-1-piperidyl]ethyl]-2-azaspiro[3.3]heptane-2-carboxylate (260 mg, 0.63 mmol, 22% yield) was obtained as a yellow solid. MS (ESI) m/z: 415.2 [M+1]⁺; ¹H NMR (400 MHz, DMSO-d6) δ 7.17 (d, J=8.4 Hz, 1H), 6.23 (d, J=8.4 Hz, 1H), 5.52 (s, 2H), 3.85 (s, 2H), 3.67 (s, 2H), 2.70 (d, J=5.2 Hz, 3H), 2.43-2.38 (m, 2H), 2.34 (d, J=10.4 Hz, 1H), 2.23 (s, 3H), 2.15 (s, 4H), 1.92 (s, 1H), 1.75 (s, 1H), 1.55 (d, J=10.0 Hz, 3H), 1.35 (s, 9H), 0.80 (d, J=6.4 Hz, 3H).

### Step 14: Preparation of tert-butyl 7-[[5-[1-[1-(2-tert-butoxycarbonyl-2-azaspiro[3.3]heptan-6-yl)ethyl]-4-piperidyl]-6-methyl-2-pyridyl]amino]-4-(7-fluoroimidazo[1,2-a]pyridin-3-yl)-1-oxo-isoindoline-2-carboxylate

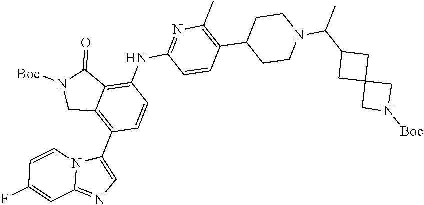 [SMILES: Cc1nc(Nc2ccc(-c3cnc4cc(F)ccn34)c3c2C(=O)N(C(=O)OC(C)(C)C)C3)ccc1C1CCN(C(C)C2CC3(C2)CN(C(=O)OC(C)(C)C)C3)CC1]

To a solution of tert-butyl 6-[1-[4-(6-amino-2-methyl-3-pyridyl)-1-piperidyl]ethyl]-2-azaspiro[3.3]heptane-2-carboxylate (260 mg, 0.63 mmol, 1 eq), tert-butyl 7-bromo-4-(7-fluoroimidazo[1,2-a]pyridin-3-yl)-1-oxo-isoindoline-2-carboxylate (280 mg, 0.63 mmol, 1 eq), potassium phosphate (400 mg, 1.88 mmol, 3 eq), (1E,4E)-1,5-diphenylpenta-1,4-dien-3-one; palladium (57 mg, 0.06 mmol, 0.1 eq) and (5-diphenylphosphanyl-9,9-dimethyl-xanthen-4-yl)-diphenyl-phosphane (36 mg, 0.06 mmol, 0.1 eq) in dioxane (10 mL) was degassed and purged with nitrogen for 3 times, and then the mixture was stirred at 100° C. for 12 h under nitrogen atmosphere. LCMS showed reactant 1 was consumed completely and desired mass was detected. The reaction mixture was filtered to remove the insoluble. The solvent was concentrated under vacuum. The residue was purified by prep-TLC (Petroleum ether:Ethyl acetate=10:1). Compound tert-butyl 7-[[5-[1-[1-(2-tert-butoxycarbonyl-2-azaspiro[3.3]heptan-6-yl)ethyl]-4-piperidyl]-6-methyl-2-pyridyl]amino]-4-(7-fluoroimidazo[1,2-a]pyridin-3-yl)-1-oxo-isoindoline-2-carboxylate (320 mg, 0.41 mmol, 65% yield) was obtained as a yellow solid. MS (ESI) m/z: 780.3 [M+1]⁺.

### Step 15: Preparation of 7-[[5-[1-[1-(2-azaspiro[3.3]heptan-6-yl)ethyl]-4-piperidyl]-6-methyl-2-pyridyl]amino]-4-(7-fluoroimidazo[1,2-a]pyridin-3-yl)isoindolin-1-one

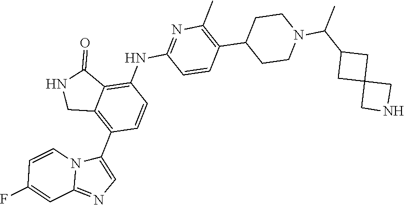 [SMILES: Cc1nc(Nc2ccc(-c3cnc4cc(F)ccn34)c3c2C(=O)NC3)ccc1C1CCN(C(C)C2CC3(CNC3)C2)CC1]

To a solution of tert-butyl 7-[[5-[1-[1-(2-tert-butoxycarbonyl-2-azaspiro[3.3]heptan-6-yl)ethyl]-4-piperidyl]-6-methyl-2-pyridyl]amino]-4-(7-fluoroimidazo[1,2-a]pyridin-3-yl)-1-oxo-isoindoline-2-carboxylate (320 mg, 0.41 mmol, 1 eq) in dichloromethane (3 mL) was added trifluoroacetic acid (1.64 g, 14.36 mmol, 1.1 mL, 35.00 eq). The mixture was stirred at 25° C. for 1 h. LCMS showed reactant 1 was consumed completely and desired mass was detected. Adjust the pH to 8 with ammonium hydroxide, the solvent was concentrated under vacuum. Adjust the pH to 5 with formic acid. The residue was purified by prep-HPLC (column: Phenomenex luna C18 150*25 mm*10 um; mobile phase: [water(FA)-ACN]; gradient: 1%-20% B over 1 min). Compound 7-[[5-[1-[1-(2-azaspiro[3.3]heptan-6-yl)ethyl]-4-piperidyl]-6-methyl-2-pyridyl]amino]-4-(7-fluoroimidazo[1,2-a]pyridin-3-yl)isoindolin-1-one (162 mg, 0.28 mmol, 68% yield) was obtained as a white solid. MS (ESI) m/z: 580.4 [M+1]⁺; ¹H NMR (400 MHz, DMSO-d6) δ 10.05 (s, 1H), 8.86 (s, 1H), 8.75 (d, J=8.4 Hz, 1H), 8.62-8.55 (m, 1H), 8.11 (s, 1H), 7.75 (d, J=8.8 Hz, 2H), 7.50 (d, J=8.4 Hz, 1H), 7.25 (dt, J=2.4, 7.6 Hz, 1H), 6.92 (d, J=8.4 Hz, 1H), 4.39 (s, 2H), 4.04 (t, J=5.6 Hz, 3H), 3.87-3.82 (m, 3H), 3.16-3.07 (m, 4H), 2.46-2.38 (m, 3H), 2.34-2.25 (m, 2H), 2.23-2.13 (m, 2H), 2.09-1.96 (m, 4H), 1.90 (d, J=12.8 Hz, 2H), 1.19 (d, J=6.4 Hz, 3H).

### Step 16: Preparation of 3-[4-fluoro-5-[6-[1-[4-[6-[[7-(7-fluoroimidazo[1,2-a]pyridin-3-yl)-3-oxo-isoindolin-4-yl]amino]-2-methyl-3-pyridyl]-1-piperidyl]ethyl]-2-azaspiro[3.3]heptan-2-yl]-1-oxo-isoindolin-2-yl]piperidine-2,6-dione

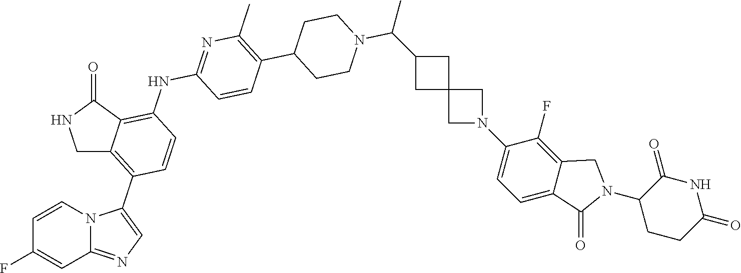 [SMILES: Cc1nc(Nc2ccc(-c3cnc4cc(F)ccn34)c3c2C(=O)NC3)ccc1C1CCN(C(C)C2CC3(C2)CN(c2ccc4c(c2F)CN(C2CCC(=O)NC2=O)C4=O)C3)CC1]

To a solution of 3-(5-bromo-4-fluoro-1-oxo-isoindolin-2-yl)piperidine-2,6-dione (71 mg, 0.21 mmol, 1 eq), 7-[[5-[1-[1-(2-azaspiro[3.3]heptan-6-yl)ethyl]-4-piperidyl]-6-methyl-2-pyridyl]amino]-4-(7-fluoroimidazo[1,2-a]pyridin-3-yl)isoindolin-1-one (120 mg, 0.21 mmol, 1 eq), bis[2-(2-pyridyl)phenyl]iridium(1+);4-tert-butyl-2-(4-tert-butyl-2-pyridyl)pyridine; hexafluorophosphate (4 mg, 0.01 mmol, 0.02 eq), dibromonickel; 1,2-dimethoxyethane (3 mg, 0.01 mmol, 0.05 eq) and 1,4-diazabicyclo[2.2.2]octane (84 mg, 0.75 mmol, 0.1 mL, 3.6 eq) in N,N-dimethylacetamide (5 mL) was degassed and purged with argon, and then the mixture was stirred at 25° C. for 16 h irradiated with a 455 nm blue LED. LCMS showed reactant 1 was consumed completely and desired mass was detected. The solvent was concentrated under vacuum. The residue was purified by prep-HPLC (column: Phenomenex luna C18 150*25 mm*10 um; mobile phase: [water(FA)-ACN]; gradient: 8%-38% B over 1 min). Compound 3-[4-fluoro-5-[6-[1-[4-[6-[[7-(7-fluoroimidazo[1,2-a]pyridin-3-yl)-3-oxo-isoindolin-4-yl]amino]-2-methyl-3-pyridyl]-1-piperidyl]ethyl]-2-azaspiro[3.3]heptan-2-yl]-1-oxo-isoindolin-2-yl]piperidine-2,6-dione (15.2 mg, 0.02 mmol, 8% yield, 89% purity) was obtained as a yellow solid. MS (ESI) m/z: 840.5 [M+1]⁺; ¹H NMR (400 MHz, DMSO-d6) δ 11.06-10.82 (m, 1H), 9.97 (s, 1H), 8.80 (s, 1H), 8.72 (d, J=8.4 Hz, 1H), 8.47-8.40 (m, 1H), 8.27 (s, 1H), 7.81 (s, 1H), 7.71 (d, J=8.4 Hz, 1H), 7.54-7.49 (m, 2H), 7.37 (d, J=8.0 Hz, 1H), 6.96 (dt, J=2.8, 7.6 Hz, 1H), 6.81 (d, J=8.4 Hz, 1H), 6.58 (t, J=8.0 Hz, 1H), 5.04 (dd, J=5.2, 13.2 Hz, 1H), 4.47-4.33 (m, 4H), 4.25 (d, J=16.8 Hz, 1H), 4.09 (s, 2H), 3.91 (s, 2H), 2.88 (d, J=11.6 Hz, 1H), 2.83-2.77 (m, 2H), 2.60 (dd, J=3.2, 4.0 Hz, 2H), 2.27 (d, J=6.0 Hz, 5H), 2.09-2.00 (m, 2H), 1.99-1.84 (m, 3H), 1.71-1.49 (m, 5H), 0.87 (d, J=6.4 Hz, 3H).

### Exemplification of Method H

### Example 298: Exemplary Synthesis of 3-(5-(4-((1-(6-((7-(7-fluoroimidazo[1,2-a]pyridin-3-yl)-3-oxoisoindolin-4-yl)amino)-2-methylpyridin-3-yl)piperidin-4-yl)oxy)piperidin-1-yl)-3-isopropyl-2-oxo-2,3-dihydro-1H-benzo[d]imidazol-1-yl)piperidine-2,6-dione

### Step 1: Preparation of tert-butyl 4-((1-(1-(2,6-dioxopiperidin-3-yl)-3-isopropyl-2-oxo-2,3-dihydro-1H-benzo[d]imidazol-5-yl)piperidin-4-yl)oxy)piperidine-1-carboxylate

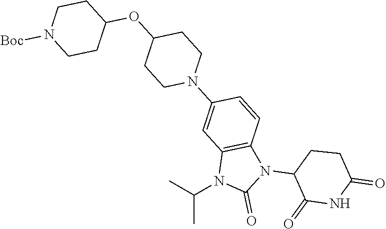 [SMILES: CC(C)n1c(=O)n(C2CCC(=O)NC2=O)c2ccc(N3CCC(OC4CCN(C(=O)OC(C)(C)C)CC4)CC3)cc21]

A mixture of tert-butyl 4-(4-piperidyloxy)piperidine-1-carboxylate (466 mg, 1.64 mmol, 1.2 eq), 3-(5-bromo-3-isopropyl-2-oxo-benzimidazol-1-yl)piperidine-2,6-dione (500 mg, 1.37 mmol, 1 eq), dicyclohexyl-[2-(2,6-diisopropoxyphenyl)phenyl]phosphane (64 mg, 0.14 mmol, 0.1 eq), [2-(2-aminophenyl)phenyl]-chloro-palladium;dicyclohexyl-[2-(2,6-diisopropoxy phenyl)phenyl]phosphane (106 mg, 0.14 mmol, 0.1 eq) and lithium bis(trimethylsilyl)amide (1 M, 6.83 mL, 5 eq) in toluene (10 mL) was degassed and purged with nitrogen for 3 times, and then the mixture was stirred at 80° C. for 2 h under nitrogen atmosphere. LCMS showed the desired mass was detected. The reaction solution was poured into aqueous acetic acid (acetic acid/water=3 ml/25 ml) at 0° C. and then saturated aqueous sodium bicarbonate solution was added and the pH was adjusted to 8. The mixture was extracted with ethyl acetate (15 ml×2). The combined organic layers were washed with brine (20 mL) and dried over anhydrous sodium sulfate, filtered and concentrated to give the crude product. The residue was purified by column chromatography (Dichloromethane:Methanol=100:1 to 10:1) followed by triturated with methyl tert-butyl ether (30 mL) at 20° C. for 120 minutes. After filtration, the filter cake was collected and dried. Compound tert-butyl 4-[[1-[1-(2,6-dioxo-3-piperidyl)-3-isopropyl-2-oxo-benzimidazol-5-yl]-4-piperidyl]oxy]piperidine-1-carboxylate (410 mg, 0.72 mmol, 53% yield) was obtained as a white solid. MS (ESI) m/z: 570.3 [M+1]⁺; ¹HNMR (400 MHz, DMSO-d₆) (11.05 (s, 1H), 6.98-6.82 (m, 2H), 6.68-6.56 (m, 1H), 5.26 (dd, J₁=5.2, J₂=12.8 Hz, 1H), 4.67-4.48 (m, 1H), 3.71-3.54 (m, 4H), 3.02 (s, 2H), 2.91-2.77 (m, 3H), 2.71-2.60 (m, 2H), 2.03-1.85 (m, 3H), 1.77 (dd, J₁=3.6, J₂=8.0 Hz, 2H), 1.62-1.52 (m, 2H), 1.45 (s, 3H), 1.44 (s, 3H), 1.40 (s, 9H), 1.36-1.25 (m, 4H).

### Step 2: Preparation of 3-(3-isopropyl-2-oxo-5-(4-(piperidin-4-yloxy)piperidin-1-yl)-2,3-dihydro-1H-benzo[d]imidazol-1-yl)piperidine-2,6-dione

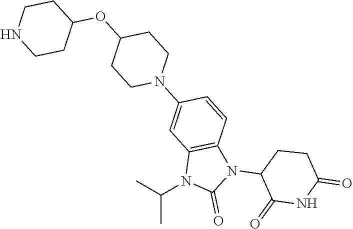 [SMILES: CC(C)n1c(=O)n(C2CCC(=O)NC2=O)c2ccc(N3CCC(OC4CCNCC4)CC3)cc21]

To a solution of tert-butyl 4-[[1-[1-(2,6-dioxo-3-piperidyl)-3-isopropyl-2-oxo-benzimidazol-5-yl]-4-piperidyl]oxy]piperidine-1-carboxylate (410 mg, 0.72 mmol, 1 eq) in dichloromethane (6 mL) was added trifluoroacetic acid (3.07 g, 26.92 mmol, 2 mL). The mixture was stirred at 20° C. for 1 h. LCMS showed the desired mass was detected. The mixture was concentrated. The crude product was triturated with methyl tert-butyl ether (20 mL) at 20° C. for 4 h. After filtration, the filter cake was collected and dried. Compound 3-[3-isopropyl-2-oxo-5-[4-(4-piperidyloxy)-1-piperidyl]benzimidazol-1-yl]piperidine-2,6-dione (331 mg, 0.70 mmol, 98% yield) was obtained as a white solid. MS (ESI) m/z: 470.2 [M+1]⁺.

### Step 3: Preparation of 3-(3-isopropyl-5-(4-((1-(2-methyl-6-nitropyridin-3-yl)piperidin-4-yl)oxy)piperidin-1-yl)-2-oxo-2,3-dihydro-1H-benzo[d]imidazol-1-yl)piperidine-2,6-dione

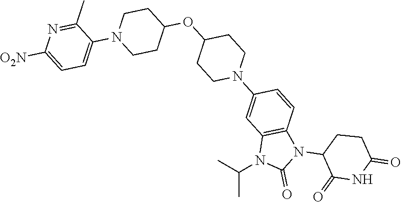 [SMILES: Cc1nc([N+](=O)[O-])ccc1N1CCC(OC2CCN(c3ccc4c(c3)n(C(C)C)c(=O)n4C3CCC(=O)NC3=O)CC2)CC1]

To a solution of 3-[3-isopropyl-2-oxo-5-[4-(4-piperidyloxy)-1-piperidyl]benzimidazol-1-yl]piperidine-2,6-dione (331 mg, 0.70 mmol, 1.1 eq), 3-fluoro-2-methyl-6-nitro-pyridine (100 mg, 0.64 mmol, 1 eq) in N,N-dimethylformamide (5 mL) was added potassium carbonate (266 mg, 1.92 mmol, 3 eq). The mixture was stirred at 40° C. for 12 h. LCMS showed the desired mass was detected. Water (20 mL) was added and the resulting mixture was extracted with ethyl acetate (2×10 mL). The combined organic layers were washed with brine (2×10 mL), dried over sodium sulfate, filtered and concentrated. The residue was purified by column chromatography (Petroleum ether/Ethyl acetate=1/0 to 0/1). Compound 3-[3-isopropyl-5-[4-[[1-(2-methyl-6-nitro-3-pyridyl)-4-piperidyl]oxy]-1-piperidyl]-2-oxo-benzimidazol-1-yl]piperidine-2,6-dione (260 mg, 0.43 mmol, 67% yield) was obtained as a white solid. MS (ESI) m/z: 606.2 [M+1]⁺; ¹HNMR (400 MHz, DMSO-d₆) δ 11.06 (s, 1H), 8.12 (d, J=8.4 Hz, 1H), 7.61 (d, J=8.8 Hz, 1H), 6.97-6.87 (m, 2H), 6.69-6.60 (m, 1H), 5.33-5.20 (m, 1H), 4.66-4.49 (m, 1H), 3.75-3.69 (m, 1H), 3.68-3.60 (m, 1H), 3.30-3.20 (m, 4H), 2.95-2.79 (m, 7H), 2.70-2.60 (m, 2H), 2.02-1.92 (m, 5H), 1.72-1.53 (m, 5H), 1.45 (d, J=6.8 Hz, 6H).

### Step 4: Preparation of 3-(5-(4-((1-(6-amino-2-methylpyridin-3-yl)piperidin-4-yl)oxy)piperidin-1-yl)-3-isopropyl-2-oxo-2,3-dihydro-1H-benzo[d]imidazol-1-yl)piperidine-2,6-dione

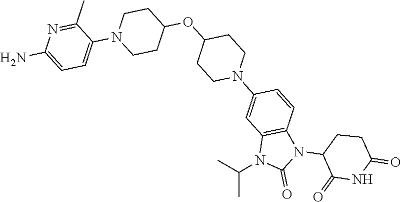 [SMILES: Cc1nc(N)ccc1N1CCC(OC2CCN(c3ccc4c(c3)n(C(C)C)c(=O)n4C3CCC(=O)NC3=O)CC2)CC1]

A solution of 3-[3-isopropyl-5-[4-[[1-(2-methyl-6-nitro-3-pyridyl)-4-piperidyl]oxy]-1-piperidyl]-2-oxo-benzimidazol-1-yl]piperidine-2,6-dione (0.26 g, 0.43 mmol, 1 eq) in tetrahydrofuran (10.4 mL). The fixed bed (5 mL) was completely packed with granular catalyst Pt/C (3 g, 1% purity) was heated to 50° C. The hydrogen back pressure regulator was adjusted to 1.5 MPa. The above solution was pumped into the fixed bed at a flow rate of {0.3 mL/min} and the flow rate of H2 was 30 mL/min. The reaction mixture was collected after running 180 mins. LCMS showed the desired mass was detected. The mixture was concentrated. The residue was purified by column chromatography (dichloromethane:methanol=1/0 to 10/1). Compound 3-[5-[4-[[1-(6-amino-2-methyl-3-pyridyl)-4-piperidyl]oxy]-1-piperidyl]-3-isopropyl-2-oxo-benzimidazol-1-yl]piperidine-2,6-dione (140 mg, 0.24 mmol, 57% yield) was obtained as a yellow solid. MS (ESI) m/z: 576.3 [M+1]⁺; ¹HNMR (400 MHz, DMSO-d₆) δ 11.05 (s, 1H), 7.18 (d, J=8.4 Hz, 1H), 6.97-6.87 (m, 2H), 6.64 (dd, J₁=2.0, J₂=8.4 Hz, 1H), 6.24 (d, J=8.4 Hz, 1H), 5.46 (s, 2H), 5.26 (dd, J₁=5.6, J₂=12.8 Hz, 1H), 4.65-4.5 (m, 1H), 3.67-3.51 (m, 2H), 3.46-3.42 (m, 1H), 2.94-2.79 (m, 5H), 2.74-2.56 (m, 5H), 2.23 (s, 3H), 1.99-1.85 (m, 5H), 1.65-1.52 (m, 4H), 1.45 (d, J=6.8 Hz, 6H).

### Step 5: Preparation of 3-(5-(4-((1-(6-((2-(2,4-dimethoxybenzyl)-7-(7-fluoroimidazo[1,2-a]pyridin-3-yl)-3-oxoisoindolin-4-yl)amino)-2-methylpyridin-3-yl)piperidin-4-yl)oxy)piperidin-1-yl)-3-isopropyl-2-oxo-2,3-dihydro-1H-benzo[d]imidazol-1-yl)piperidine-2,6-dione

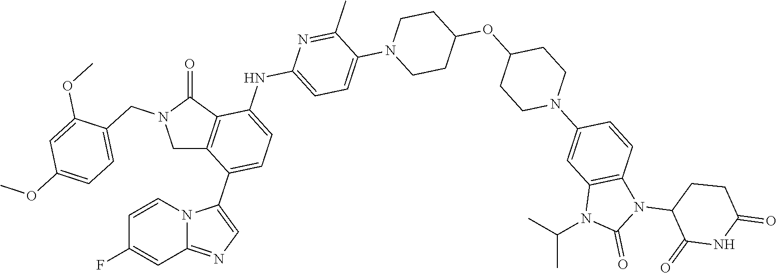 [SMILES: COc1ccc(CN2Cc3c(-c4cnc5cc(F)ccn45)ccc(Nc4ccc(N5CCC(OC6CCN(c7ccc8c(c7)n(C(C)C)c(=O)n8C7CCC(=O)NC7=O)CC6)CC5)c(C)n4)c3C2=O)c(OC)c1]

A mixture of 3-[5-[4-[[1-(6-amino-2-methyl-3-pyridyl)-4-piperidyl]oxy]-1-piperidyl]-3-isopropyl-2-oxo-benzimidazol-1-yl]piperidine-2,6-dione (140 mg, 0.24 mmol, 1 eq), 7-chloro-2-(2,4-dimethoxybenzyl)-4-(7-fluoroimidazo[1,2-a]pyridin-3-yl)isoindolin-1-one (110 mg, 0.24 mmol, 1 eq), [2-(2-aminophenyl)phenyl]-methylsulfonyloxy-palladium;dicyclohexyl-[2-(2,6-diisopropoxyphenyl)phenyl]phosphane, cesium carbonate (198 mg, 0.61 mmol, 2.5 eq) in dioxane (3 mL) was degassed and purged with nitrogen for 3 times, and then the mixture was stirred at 100° C. for 12 h under nitrogen atmosphere. LCMS showed the desired mass was detected. Water (20 mL) was added and the resulting mixture was extracted with ethyl acetate (2×10 mL). The combined organic layers were washed with brine (2×10 mL), dried over sodium sulfate, filtered and concentrated. The residue was purified by prep-TLC (petroleum ether:ethyl acetate=0:1). Compound 3-(5-(4-((1-(6-((2-(2,4-dimethoxybenzyl)-7-(7-fluoroimidazo[1,2-a]pyridin-3-yl)-3-oxoisoindolin-4-yl)amino)-2-methylpyridin-3-yl)piperidin-4-yl)oxy)piperidin-1-yl)-3-isopropyl-2-oxo-2,3-dihydro-1H-benzo[d]imidazol-1-yl)piperidine-2,6-dione (70 mg, 0.07 mmol, 29% yield) was obtained as a yellow solid. MS (ESI) m/z: 991.4 [M+1]⁺; ¹HNMR (400 MHz, DMSO-d₆) δ 11.17-11.02 (m, 1H), 9.92 (s, 1H), 8.73 (d, J=8.4 Hz, 1H), 8.47-8.40 (m, 1H), 7.79 (s, 1H), 7.73 (d, J=8.4 Hz, 1H), 7.57-7.47 (m, 2H), 7.15 (d, J=8.4 Hz, 1H), 7.04-6.93 (m, 3H), 6.89 (d, J=8.4 Hz, 1H), 6.68 (dd, J₁=1.6, J₂=8.4 Hz, 1H), 6.60 (d, J=2.4 Hz, 1H), 6.51 (dd, J₁=2.4, J₂=8.4 Hz, 1H), 5.35-5.25 (m, 1H), 4.71-4.55 (m, 3H), 4.39 (s, 2H), 3.81 (s, 3H), 3.77 (s, 3H), 3.72-3.61 (m, 2H), 3.51-3.47 (m, 1H), 3.09-2.99 (m, 2H), 2.96-2.84 (m, 3H), 2.78-2.70 (m, 3H), 2.69-2.65 (m, 1H), 2.50 (s, 3H), 2.06-1.95 (m, 6H), 1.72-1.61 (m, 4H), 1.49 (d, J=6.8 Hz, 6H).

### Step 6: Preparation of 3-(5-(4-((1-(6-((7-(7-fluoroimidazo[1,2-a]pyridin-3-yl)-3-oxoisoindolin-4-yl)amino)-2-methylpyridin-3-yl)piperidin-4-yl)oxy)piperidin-1-yl)-3-isopropyl-2-oxo-2,3-dihydro-1H-benzo[d]imidazol-1-yl)piperidine-2,6-dione

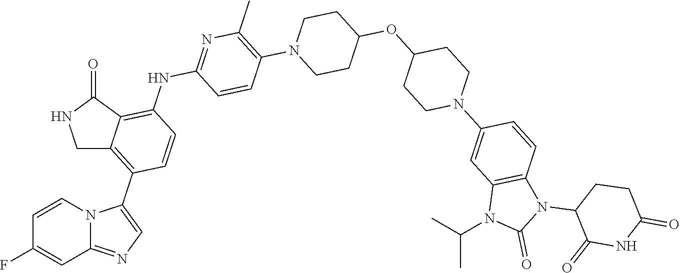 [SMILES: Cc1nc(Nc2ccc(-c3cnc4cc(F)ccn34)c3c2C(=O)NC3)ccc1N1CCC(OC2CCN(c3ccc4c(c3)n(C(C)C)c(=O)n4C3CCC(=O)NC3=O)CC2)CC1]

To a solution of 3-[5-[4-[[1-[6-[[2-[(2,4-dimethoxyphenyl)methyl]-7-(7-fluoroimidazo[1,2-a]pyridin-3-yl)-3-oxo-isoindolin-4-yl]amino]-2-methyl-3-pyridyl]-4-piperidyl]oxy]-1-piperidyl]-3-isopropyl-2-oxo-benzimidazol-1-yl]piperidine-2,6-dione (60 mg, 0.06 mmol, 1 eq) in trifluoroacetic acid (1 mL). The mixture was stirred at 60° C. for 16 h. LCMS showed the desired mass was detected. The pH was adjusted to 7-8 with saturated aqueous solution of sodium bicarbonate, and the resulting mixture was extracted with dichloromethane (3×10 mL). The combined organic layers were dried over sodium sulfate, filtered and concentrated. The residue was purified by prep-HPLC (column: Phenomenex luna C18 150*25 mm*10 um; mobile phase: [water(FA)-ACN]; gradient: 2%-32% B over 15 min). Compound 3-[5-[4-[[1-[6-[[7-(7-fluoroimidazo[1,2-a]pyridin-3-yl)-3-oxo-isoindolin-4-yl]amino]-2-methyl-3-pyridyl]-4-piperidyl]oxy]-1-piperidyl]-3-isopropyl-2-oxo-benzimidazol-1-yl]piperidine-2,6-dione (23 mg, 0.03 mmol, 45% yield, 99.0% purity) was obtained as a off-white solid. MS (ESI) m/z: 841.5 [M+1]⁺; ¹H NMR (400 MHz, DMSO-d₆) δ 11.11 (s, 1H), 9.99 (s, 1H), 8.85 (s, 1H), 8.72 (d, J=8.4 Hz, 1H), 8.56-8.44 (m, 1H), 7.88 (s, 1H), 7.77 (d, J=8.4 Hz, 1H), 7.58 (dd, J₁=2.4, J₂=10.0 Hz, 1H), 7.54-7.48 (m, 1H), 7.08-6.94 (m, 3H), 6.89 (d, J=8.4 Hz, 1H), 6.74-6.67 (m, 1H), 5.33 (dd, J₁=5.2, J₂=12.8 Hz, 1H), 4.70-4.60 (m, 1H), 4.44 (s, 2H), 3.78-3.62 (m, 2H), 3.48 (s, 2H), 3.10-3.00 (m, 2H), 2.98-2.86 (m, 3H), 2.81-2.72 (m, 3H), 2.71-2.66 (m, 1H), 2.52 (s, 3H), 2.11-1.93 (m, 5H), 1.75-1.60 (m, 4H), 1.51 (d, J=6.8 Hz, 6H).

### Exemplification of Method I

### Example 299: Exemplary Synthesis of 3-[5-[7-[6-[[7-(7-fluoroimidazo[1,2-a]pyridin-3-yl)-3-oxo-isoindolin-4-yl]amino]-2-methyl-3-pyridyl]-2,7-diazaspiro[3.5]nonan-2-yl]-3-methyl-2-oxo-benzimidazol-1-yl]piperidine-2,6-dione

### Step 1: Preparation of tert-butyl 2-[1-(2,6-dioxo-3-piperidyl)-3-methyl-2-oxo-benzimidazol-5-yl]-2,7-diazaspiro[3.5]nonane-7-carboxylate

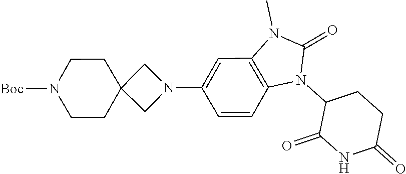 [SMILES: Cn1c(=O)n(C2CCC(=O)NC2=O)c2ccc(N3CC4(CCN(C(=O)OC(C)(C)C)CC4)C3)cc21]

A mixture of 3-(5-bromo-3-methyl-2-oxo-benzimidazol-1-yl)piperidine-2,6-dione (1.50 g, 4.44 mmol, 1.0 eq), tert-butyl 2,7-diazaspiro[3.5]nonane-7-carboxylate (1.10 g, 4.88 mmol, 1.1 eq), [2-(2-aminophenyl)phenyl]-chloro-palladium;dicyclohexyl-[2-(2,6-diisopropoxyphenyl) phenyl]phosphane (344 mg, 0.44 mmol, 0.1 eq) and dicyclohexyl-[2-(2,6-diisopropoxyphenyl)phenyl]phosphane (103 mg, 0.22 mmol, 0.05 eq) in toluene (40 mL) was degassed and purged with nitrogen for 3 times, then [bis(trimethylsilyl)amino]lithium (1.0 M, 22.1 mL, 22.1 mmol, 5.0 eq) was added and the resulting mixture was stirred at 100° C. for 1 hour under nitrogen atmosphere. LC-MS showed 32.3% of the desired mass was detected. The reaction was cooled to 25° C. and quenched by with formic acid until pH=6. The solid thus formed was collected by filtration and triturated with ethyl acetate (10 mL) for 0.5 hour to afford tert-butyl 2-[1-(2,6-dioxo-3-piperidyl)-3-methyl-2-oxo-benzimidazol-5-yl]-2,7-diazaspiro[3.5]nonane-7-carboxylate (1.48 g, 3.06 mmol, 69% yield) as a gray solid. MS (ESI) m/z: 484.2 [M+1]⁺. ¹HNMR (400 Hz, DMSO-d₆) δ 6.89 (d, J=8.4 Hz, 1H), 6.29 (d, J=1.6 Hz, 1H), 6.09 (dd, J=8.4, 1.6 Hz, 1H), 5.25 (dd, J=12.8, 5.2 Hz, 1H), 3.57-3.54 (m, 4H), 3.34-3.32 (m, 4H), 3.26 (s, 3H), 2.95-2.78 (m, 2H), 2.72-2.60 (m, 2H), 1.73-1.59 (m, 4H), 1.39 (s, 9H).

### Step 2: Preparation of 3-[5-(2,7-diazaspiro[3.5]nonan-2-yl)-3-methyl-2-oxo-benzimidazol-1-yl]piperidine-2,6-dione.TFA

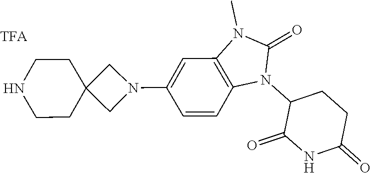 [SMILES: Cn1c(=O)n(C2CCC(=O)NC2=O)c2ccc(N3CC4(CCNCC4)C3)cc21]

To a solution of tert-butyl 2-[1-(2,6-dioxo-3-piperidyl)-3-methyl-2-oxo-benzimidazol-5-yl]-2,7-diazaspiro[3.5]nonane-7-carboxylate (650 mg, 1.34 mmol, 1.0 eq) in dichloromethane (3.0 mL) was added trifluoroacetic acid (1.0 mL). The mixture was stirred at 25° C. for 1 hour. LC-MS indicated 69.2% of desired product mass was detected. The mixture was concentrated under vacuum to obtain 3-[5-(2,7-diazaspiro[3.5]nonan-2-yl)-3-methyl-2-oxo-benzimidazol-1-yl]piperidine-2,6-dione (650 mg, crude, trifluoroacetate) as brown oil. MS (ESI) m/z: 384.2 [M+1]⁺.

### Step 3: Preparation of 3-[3-methyl-5-[7-(2-methyl-6-nitro-3-pyridyl)-2,7-diazaspiro[3.5]nonan-2-yl]-2-oxo-benzimidazol-1-yl]piperidine-2,6-dione

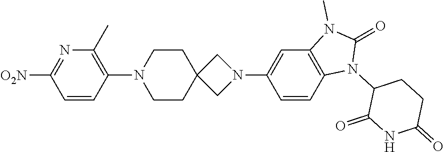 [SMILES: Cc1nc([N+](=O)[O-])ccc1N1CCC2(CC1)CN(c1ccc3c(c1)n(C)c(=O)n3C1CCC(=O)NC1=O)C2]

A mixture of 3-[5-(2,7-diazaspiro[3.5]nonan-2-yl)-3-methyl-2-oxo-benzimidazol-1-yl]piperidine-2,6-dione (350 mg, 0.70 mmol, 1.0 eq, trifluoroacetate), 3-fluoro-2-methyl-6-nitro-pyridine (164 mg, 1.06 mmol, 1.5 eq), and potassium carbonate (291 mg, 2.11 mmol, 3.0 eq) in N,N-dimethyl-formamide (4.0 mL) was stirred at 40° C. for 12 hours under nitrogen atmosphere. LC-MS indicated 72.1% of the desired product mass was detected. The reaction was taken up in water (30 mL) and filtered to collect the solid. It was triturated with petroleum ether and ethyl acetate (4:1, 50 mL) to afford 3-[3-methyl-5-[7-(2-methyl-6-nitro-3-pyridyl)-2,7-diazaspiro[3.5]nonan-2-yl]-2-oxo-benzimidazol-1-yl]piperidine-2,6-dione (195 mg, 0.37 mmol, 53% yield) as a yellow solid, which was confirmed by ¹HNMR (EW51601-59-P1A1). MS (ESI) m/z: 520.3 [M+1]⁺. ¹HNMR (400 MHz, DMSO-d₆) δ 11.35-10.75 (m, 1H), 8.25-8.17 (m, 1H), 7.68 (d, J=8.8 Hz, 1H), 6.95 (d, J=8.4 Hz, 1H), 6.39 (d, J=2.0 Hz, 1H), 6.19 (dd, J=8.4, 2.0 Hz, 1H), 5.36-5.13 (m, 1H), 3.69 (s, 4H), 3.35 (s, 3H), 3.14-3.04 (m, 4H), 3.00-2.78 (m, 2H), 2.76-2.64 (m, 2H), 2.59 (s, 3H), 2.04-1.95 (m, 4H).

### Step 4: Preparation of 3-[5-[7-(6-amino-2-methyl-3-pyridyl)-2,7-diazaspiro[3.5]nonan-2-yl]-3-methyl-2-oxo-benzimidazol-1-yl]piperidine-2,6-dione

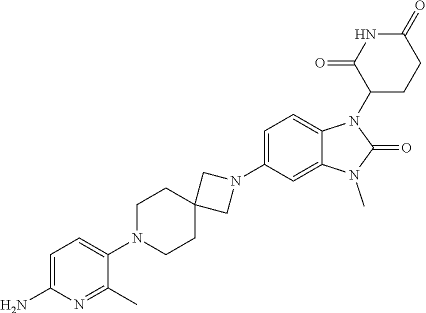 [SMILES: Cc1nc(N)ccc1N1CCC2(CC1)CN(c1ccc3c(c1)n(C)c(=O)n3C1CCC(=O)NC1=O)C2]

To a solution of 3-[3-methyl-5-[7-(2-methyl-6-nitro-3-pyridyl)-2,7-diazaspiro[3.5]nonan-2-yl]-2-oxo-benzimidazol-1-yl]piperidine-2,6-dione (195 mg, 0.37 mmol, 1.0 eq) in tetrahydrofuran (6.0 mL) was added dry palladium/carbon (50 mg, 0.37 mmol, 10% purity, 1.0 eq) under nitrogen atmosphere. The suspension was degassed under vacuum and purged with hydrogen several times. The mixture was stirred under hydrogen atmosphere (50 psi) at 25° C. for 12 hours. LC-MS showed 89.8% of the desired product mass was detected. The reaction was filtered through a pad of celite and the filtrate was concentrated to obtain 3-[5-[7-(6-amino-2-methyl-3-pyridyl)-2,7-diazaspiro[3.5]nonan-2-yl]-3-methyl-2-oxo-benzimidazol-1-yl]piperidine-2,6-dione (65 mg, 0.13 mmol, 35% yield) as a yellow solid. MS (ESI) m/z: 490.3 [M+1]⁺. ¹HNMR (400 MHz, DMSO-d₆) δ 11.25-10.84 (m, 1H), 7.26-7.19 (m, 1H), 7.02-6.90 (m, 1H), 6.38 (d, J=2.0 Hz, 1H), 6.34-6.29 (m, 1H), 6.18 (dd, J=8.4, 2.0 Hz, 1H), 5.52 (s, 2H), 5.33 (dd, J=13.2, 5.6 Hz, 1H), 3.67-3.66 (m, 4H), 3.35 (s, 3H), 2.77-2.63 (m, 6H), 2.30 (s, 3H), 2.28-2.23 (m, 2H), 1.98-1.88 (m, 4H).

### Step 5: Preparation of tert-butyl 7-[[5-[2-[1-(2,6-dioxo-3-piperidyl)-3-methyl-2-oxo-benzimidazol-5-yl]-2,7-diazaspiro[3.5]nonan-7-yl]-6-methyl-2-pyridyl]amino]-4-(7-fluoroimidazo[1,2-a]pyridin-3-yl)-1-oxo-isoindoline-2-carboxylate

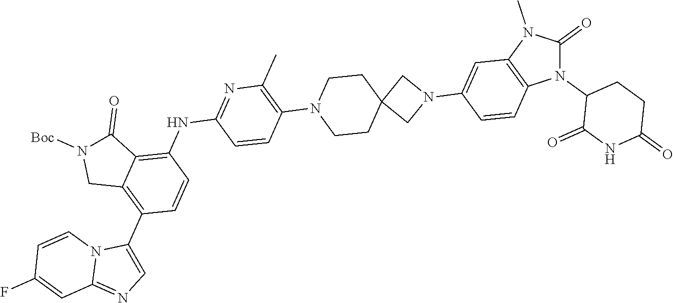 [SMILES: Cc1nc(Nc2ccc(-c3cnc4cc(F)ccn34)c3c2C(=O)N(C(=O)OC(C)(C)C)C3)ccc1N1CCC2(CC1)CN(c1ccc3c(c1)n(C)c(=O)n3C1CCC(=O)NC1=O)C2]

A mixture of 3-[5-[7-(6-amino-2-methyl-3-pyridyl)-2,7-diazaspiro[3.5]nonan-2-yl]-3-methyl-2-oxo-benzimidazol-1-yl]piperidine-2,6-dione (65 mg, 0.13 mmol, 1.0 eq), tert-butyl 7-bromo-4-(7-fluoroimidazo[1,2-a]pyridin-3-yl)-1-oxo-isoindoline-2-carboxylate (59 mg, 0.13 mmol, 1.0 eq), potassium phosphate (70 mg, 0.33 mmol, 2.5 eq), (5-diphenylphosphanyl-9,9-dimethyl-xanthen-4-yl)-diphenyl-phosphane (11 mg, 0.02 mmol, 0.1 eq) and (1E,4E)-1,5-diphenylpenta-1,4-dien-3-one palladium (12 mg, 0.02 mmol, 0.1 eq) in dioxane (1.0 mL) was degassed and purged with nitrogen for 3 times, and then the mixture was stirred at 100° C. for 3 hours under nitrogen atmosphere. LC-MS indicated 43.0% of the desired product mass was detected. The reaction was filtered and concentrated under vacuum to obtain a residue. It was purified by prep-TLC (10% methanol in dichloromethane) to afford tert-butyl 7-[[5-[2-[1-(2,6-dioxo-3-piperidyl)-3-methyl-2-oxo-benzimidazol-5-yl]-2,7-diazaspiro[3.5]nonan-7-yl]-6-methyl-2-pyridyl]amino]-4-(7-fluoroimidazo[1,2-a]pyridin-3-yl)-1-oxo-isoindoline-2-carboxylate (44 mg, 43.28 μmol, 32% yield, 84.1% purity) as a yellow solid. MS (ESI) m/z: 855.4 [M+1]⁺.

### Step 6: Preparation of 3-[5-[7-[6-[[7-(7-fluoroimidazo[1,2-a]pyridin-3-yl)-3-oxo-isoindolin-4-yl]amino]-2-methyl-3-pyridyl]-2,7-diazaspiro[3.5]nonan-2-yl]-3-methyl-2-oxo-benzimidazol-1-yl]piperidine-2,6-dione

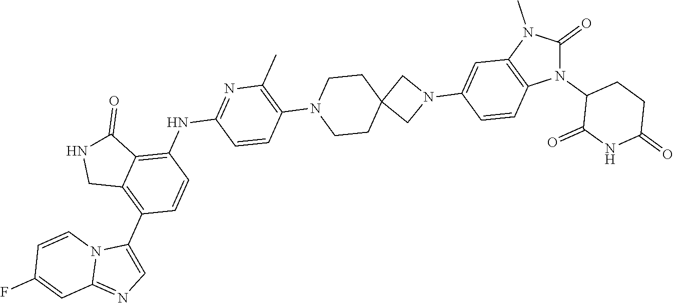 [SMILES: Cc1nc(Nc2ccc(-c3cnc4cc(F)ccn34)c3c2C(=O)NC3)ccc1N1CCC2(CC1)CN(c1ccc3c(c1)n(C)c(=O)n3C1CCC(=O)NC1=O)C2]

To a solution of tert-butyl 7-[[5-[2-[1-(2,6-dioxo-3-piperidyl)-3-methyl-2-oxo-benzimidazol-5-yl]-2,7-diazaspiro[3.5]nonan-7-yl]-6-methyl-2-pyridyl]amino]-4-(7-fluoroimidazo[1,2-a]pyridin-3-yl)-1-oxo-isoindoline-2-carboxylate (44 mg, 0.05 mmol, 1.0 eq) in dichloromethane (0.3 mL) was added trifluoroacetic acid (0.1 mL). The mixture was stirred at 25° C. for 0.5 hour. LC-MS indicated 76.2% of the desired product mass was detected. The reaction was concentrated in vacuum to obtain the residue. It was purified by Prep-HPLC (column: Welch Xtimate C18 150*25 mm*5 um; mobile phase: [water (formic acid)-acetonitrile]; gradient: 10%-40% B over 10 min) to afford 3-[5-[7-[6-[[7-(7-fluoroimidazo[1,2-a]pyridin-3-yl)-3-oxo-isoindolin-4-yl]amino]-2-methyl-3-pyridyl]-2,7-diazaspiro[3.5]nonan-2-yl]-3-methyl-2-oxo-benzimidazol-1-yl]piperidine-2,6-dione (13.6 mg, 17.59 μmol, 34% yield, 97.60% purity) as a white solid. MS (ESI) m/z: 755.5 [M+1]⁺; ¹HNMR (400 MHz, DMSO-d₆) δ 11.16-10.98 (m, 1H), 9.95 (s, 1H), 8.80 (s, 1H), 8.67 (d, J=8.4 Hz, 1H), 8.55-8.41 (m, 1H), 7.82 (s, 1H), 7.72 (d, J=8.4 Hz, 1H), 7.56-7.40 (m, 2H), 7.03-6.79 (m, 3H), 6.34 (d, J=1.6 Hz, 1H), 6.19-6.08 (m, 1H), 5.37-5.19 (m, 1H), 4.39 (s, 2H), 3.63 (s, 4H), 3.30 (s, 3H), 2.96-2.86 (m, 1H), 2.86-2.77 (m, 4H), 2.75-2.66 (m, 1H), 2.64-2.58 (m, 1H), 2.48 (s, 3H), 2.03-1.87 (m, 5H).

### Exemplification of Method J

### Example 300: Exemplary Synthesis of 3-[5-[4-[1-[6-[[7-(7-fluoroimidazo[1,2-a]pyridin-3-yl)-3-oxo-isoindolin-4-yl]amino]-2-methyl-3-pyridyl]-4-piperidyl]-1-piperidyl]-3-methyl-2-oxo-benzimidazol-1-yl]piperidine-2,6-dione

### Step 1: Preparation of tert-butyl 4-[1-[1-(2,6-dioxo-3-piperidyl)-3-methyl-2-oxo-benzimidazol-5-yl]-4-piperidyl]piperidine-1-carboxylate

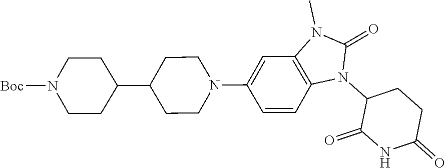 [SMILES: Cn1c(=O)n(C2CCC(=O)NC2=O)c2ccc(N3CCC(C4CCN(C(=O)OC(C)(C)C)CC4)CC3)cc21]

A mixture of 3-(5-bromo-3-methyl-2-oxo-benzimidazol-1-yl)piperidine-2,6-dione (1 g, 2.96 mmol, 1 eq) in toluene (30 mL) was added tert-butyl 4-(4-piperidyl)piperidine-1-carboxylate (873 mg, 3.25 mmol, 1.1 eq), 2-dicyclohexylphosphino-2′,6′-diisopropoxybiphenyl (69 mg, 0.14 mmol, 0.05 eq) and chloro(2-dicyclohexylphosphino-2′,6′-diisopropoxy-1,1′-biphenyl)[2-(2′-amino-1,1′-biphenyl)]palladium(II) (229 mg, 0.29 mmol, 0.1 eq) was added to the mixture. The mixture was degassed and purged with nitrogen for 3 times. Then hexamethyldisiliconyl amine lithium (1 M, 14.8 mL, 5 eq) was added to the mixture. The reaction was stirred at 100° C. for 3 h under nitrogen atmosphere. LC-MS showed Reactant 1 was consumed completely. The reaction mixture was quenched by addition formic acid till pH=6, and then diluted with aqueous sodium hydrogen carbonate 50 mL and extracted with dichloromethane (100 mL×3). The combined organic layers were dried over anhydrous sodium sulfate, filtered and concentrated under reduced pressure to give a residue. The crude product was triturated with the solution (ethyl acetate/tert-butyl methyl ether=1/1) for 30 min. Compound tert-butyl 4-[1-[1-(2,6-dioxo-3-piperidyl)-3-methyl-2-oxo-benzimidazol-5-yl]-4-piperidyl]piperidine-1-carboxylate (660 mg, 1.26 mmol, 42% yield) was obtained as a dark purple solid. MS (ESI) m/z: 526.3 [M+1]⁺; ¹HNMR (400 MHz, DMSO-d₆) δ 6.92 (d, J=8.4 Hz, 1H), 6.82 (d, J=2.0 Hz, 1H), 6.62 (dd, J=2.0, 8.4 Hz, 1H), 5.34-5.21 (m, 1H), 3.97 (d, J=11.2 Hz, 2H), 3.66-3.56 (m, 3H), 3.27 (s, 3H), 2.94-2.83 (m, 1H), 2.69-2.61 (m, 3H), 2.57 (s, 1H), 1.99 (s, 1H), 1.75 (d, J=12.0 Hz, 2H), 1.68 (d, J=12.0 Hz, 2H), 1.39 (s, 9H), 1.37 (s, 1H), 1.34-1.24 (m, 3H), 1.17 (s, 1H), 1.08-0.98 (m, 2H).

### Step 2: Preparation of 3-[3-methyl-2-oxo-5-[4-(4-piperidyl)-1-piperidyl]benzimidazol-1-yl]piperidine-2,6-dione

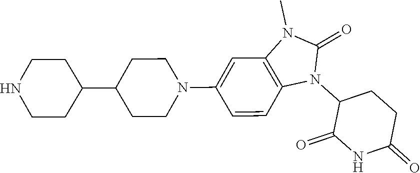 [SMILES: Cn1c(=O)n(C2CCC(=O)NC2=O)c2ccc(N3CCC(C4CCNCC4)CC3)cc21]

To a solution of tert-butyl 4-[1-[1-(2,6-dioxo-3-piperidyl)-3-methyl-2-oxo-benzimidazol-5-yl]-4-piperidyl]piperidine-1-carboxylate (200 mg, 0.38 mmol, 1 eq) in dichloromethane (2 mL) was added trifluoroacetic acid (1.54 g, 13.46 mmol, 1 mL, 35.38 eq). The mixture was stirred at 20° C. for 1 h. LC-MS showed Reactant 1 was consumed completely. The reaction was concentrated in reduced pressure at 45° C. The residue was used into the next step without further purification. Compound 3-[3-methyl-2-oxo-5-[4-(4-piperidyl)-1-piperidyl]benzimidazol-1-yl]piperidine-2,6-dione (205 mg, crude, trifluoroacetate) was obtained as a dark purple oil. MS (ESI) m/z: 426.3 [M+1]⁺.

### Step 3: Preparation of 3-[3-methyl-5-[4-[1-(2-methyl-6-nitro-3-pyridyl)-4-piperidyl]-1-piperidyl]-2-oxo-benzimidazol-1-yl]piperidine-2,6-dione

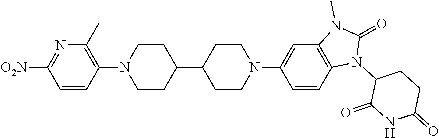 [SMILES: Cc1nc([N+](=O)[O-])ccc1N1CCC(C2CCN(c3ccc4c(c3)n(C)c(=O)n4C3CCC(=O)NC3=O)CC2)CC1]

To a solution of 3-[3-methyl-2-oxo-5-[4-(4-piperidyl)-1-piperidyl]benzimidazol-1-yl]piperidine-2,6-dione (205 mg, 0.37 mmol, 1 eq, trifluoroacetate) in dimethyl formamide (4 mL) was added potassium carbonate (157 mg, 1.14 mmol, 3 eq) and 3-fluoro-2-methyl-6-nitro-pyridine (88 mg, 0.56 mmol, 1.5 eq). The mixture was stirred at 40° C. for 12 h. LC-MS showed Reactant 1 was consumed completely. The reaction mixture was diluted with ethyl acetate 5 mL and water 10 mL, then extracted with ethyl acetate (50 mL×3). The combined organic layers were washed with brine 50 mL, dried over anhydrous sodium sulfate, filtered and concentrated under reduced pressure to give a residue. The residue was purified by flash silica gel chromatography (ISCO®; 12 g SepaFlash® Silica Flash Column, Eluent of 0˜100% Ethyl acetate/Petroleum ethergradient). Compound 3-[3-methyl-5-[4-[1-(2-methyl-6-nitro-3-pyridyl)-4-piperidyl]-1-piperidyl]-2-oxo-benzimidazol-1-yl]piperidine-2,6-dione (119 mg, 0.21 mmol, 55% yield) was obtained as a white solid. MS (ESI) m/z: 562.3 [M+1]⁺; ¹HNMR (400 MHz, CHLOROFORM-d) δ 8.14 (s, 1H), 8.09 (d, J=8.4 Hz, 1H), 7.37 (d, J=8.4 Hz, 1H), 6.71 (s, 2H), 5.20 (dd, J=5.2, 12.4 Hz, 1H), 3.63 (d, J=11.6 Hz, 2H), 3.42 (s, 3H), 3.37 (d, J=12.0 Hz, 2H), 2.97-2.89 (m, 1H), 2.88-2.78 (m, 1H), 2.76-2.67 (m, 4H), 2.62 (s, 3H), 2.24 (m, 1H), 1.92 (t, J=10.4 Hz, 4H), 1.66-1.45 (m, 6H), 1.40-1.31 (m, 2H).

### Step 4: Preparation of 3-[5-[4-[1-(6-amino-2-methyl-3-pyridyl)-4-piperidyl]-1-piperidyl]-3-methyl-2-oxo-benzimidazol-1-yl]piperidine-2,6-dione

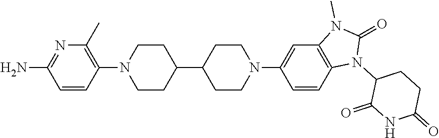 [SMILES: Cc1nc(N)ccc1N1CCC(C2CCN(c3ccc4c(c3)n(C)c(=O)n4C3CCC(=O)NC3=O)CC2)CC1]

To a solution of 3-[3-methyl-5-[4-[1-(2-methyl-6-nitro-3-pyridyl)-4-piperidyl]-1-piperidyl]-2-oxo-benzimidazol-1-yl]piperidine-2,6-dione (174 mg, 0.30 mmol, 1 eq) in tetrahydrofuran (6 mL) was added palladium on activated carbon catalyst (50 mg, 10% purity) under nitrogen. The suspension was degassed under vacuum and purged with hydrogen several times. The mixture was stirred under hydrogen (50 psi) at 25° C. for 12 h. LC-MS showed Reactant 1 was consumed completely. The reaction mixture was filtered and the filter was concentrated. The crude product was triturated with the solvent (dichloromethane/Methanol/tert-butyl methyl ether=10/1/50) for 30 min. Compound 3-[5-[4-[1-(6-amino-2-methyl-3-pyridyl)-4-piperidyl]-1-piperidyl]-3-methyl-2-oxo-benzimidazol-1-yl]piperidine-2,6-dione (100 mg, 0.18 mmol, 60% yield) was obtained as a yellow solid. MS (ESI) m/z: 532.3 [M+1]⁺; ¹HNMR (400 MHz, DMSO-d₆) δ 11.07 (s, 1H), 7.16 (d, J=8.4 Hz, 1H), 6.93 (d, J=8.4 Hz, 1H), 6.82 (d, J=2.0 Hz, 1H), 6.63 (dd, J=2.0, 8.8 Hz, 1H), 6.24 (d, J=8.4 Hz, 1H), 5.43 (s, 2H), 5.29 (dd, J=5.2, 12.8 Hz, 1H), 3.63 (d, J=11.2 Hz, 3H), 3.30 (s, 3H), 2.88 (d, J=11.2 Hz, 2H), 2.68-2.62 (m, 2H), 2.59-2.53 (m, 4H), 2.22 (s, 3H), 2.01-1.95 (m, 1H), 1.78 (t, J=14.0 Hz, 4H), 1.38-1.29 (m, 4H), 1.23-1.14 (m, 2H).

### Step 5: Preparation of tert-butyl 7-[[5-[4-[1-[1-(2,6-dioxo-3-piperidyl)-3-methyl-2-oxo-benzimidazol-5-yl]-4-piperidyl]-1-piperidyl]-6-methyl-2-pyridyl]amino]-4-(7-fluoroimidazo[1,2-a]pyridin-3-yl)-1-oxo-isoindoline-2-carboxylate

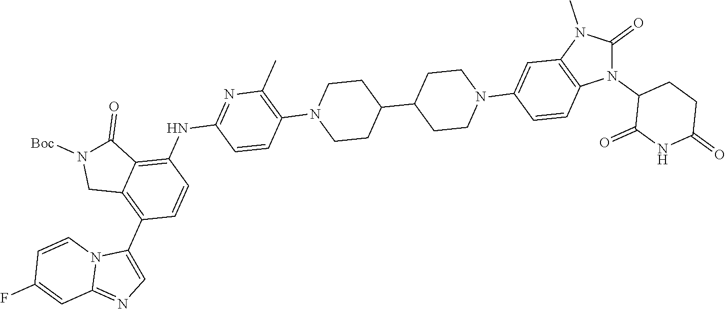 [SMILES: Cc1nc(Nc2ccc(-c3cnc4cc(F)ccn34)c3c2C(=O)N(C(=O)OC(C)(C)C)C3)ccc1N1CCC(C2CCN(c3ccc4c(c3)n(C)c(=O)n4C3CCC(=O)NC3=O)CC2)CC1]

A mixture of 3-[5-[4-[1-(6-amino-2-methyl-3-pyridyl)-4-piperidyl]-1-piperidyl]-3-methyl-2-oxo-benzimidazol-1-yl]piperidine-2,6-dione (40 mg, 0.07 mmol, 1 eq), tert-butyl 7-bromo-4-(7-fluoroimidazo[1,2-a]pyridin-3-yl)-1-oxo-isoindoline-2-carboxylate (43 mg, 0.09 mmol, 1.28 eq), potassium phosphate (40 mg, 0.18 mmol, 2.5 eq), 4,5-bis(diphenylphosphino)-9,9-dimethylxanthene (7 mg, 0.01 mmol, 0.15 eq) in dioxane (1 mL) was degassed and purged with nitrogen for 3 times, and then tris(dibenzylideneacetone)dipalladium(0) (7 mg, 0.01 mmol, 0.1 eq) was added to the mixture. The mixture was stirred at 100° C. for 18 h under nitrogen atmosphere. LC-MS showed Reactant 1 was consumed completely. The reaction mixture was filtered and the filter was concentrated. The residue was purified by prep-TLC (dichloromethane/Methanol=10/1). Compound tert-butyl 7-[[5-[4-[1-[1-(2,6-dioxo-3-piperidyl)-3-methyl-2-oxo-benzimidazol-5-yl]-4-piperidyl]-1-piperidyl]-6-methyl-2-pyridyl]amino]-4-(7-fluoroimidazo[1,2-a]pyridin-3-yl)-1-oxo-isoindoline-2-carboxylate (46 mg, 0.05 mmol, 68% yield) was obtained as a yellow solid. MS (ESI) m/z: 897.4 [M]⁺.

### Step 6: Preparation of 3-[5-[4-[1-[6-[[7-(7-fluoroimidazo[1,2-a]pyridin-3-yl)-3-oxo-isoindolin-4-yl]amino]-2-methyl-3-pyridyl]-4-piperidyl]-1-piperidyl]-3-methyl-2-oxo-benzimidazol-1-yl]piperidine-2,6-dione

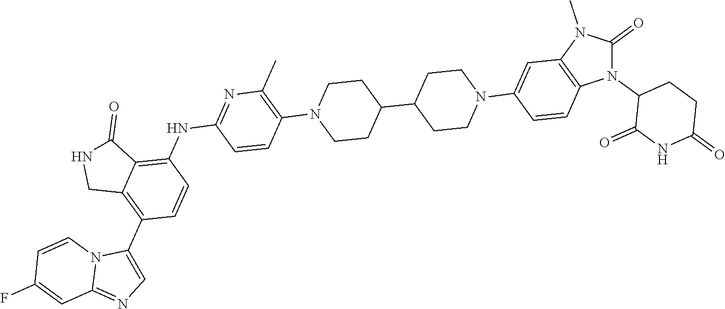 [SMILES: Cc1nc(Nc2ccc(-c3cnc4cc(F)ccn34)c3c2C(=O)NC3)ccc1N1CCC(C2CCN(c3ccc4c(c3)n(C)c(=O)n4C3CCC(=O)NC3=O)CC2)CC1]

To a solution of tert-butyl 7-[[5-[4-[1-[1-(2,6-dioxo-3-piperidyl)-3-methyl-2-oxo-benzimidazol-5-yl]-4-piperidyl]-1-piperidyl]-6-methyl-2-pyridyl]amino]-4-(7-fluoroimidazo[1,2-a]pyridin-3-yl)-1-oxo-isoindoline-2-carboxylate (46 mg, 0.05 mmol, 1 eq) in dichloromethane (1.5 mL) was added trifluoroacetic acid (767 mg, 6.73 mmol, 0.5 mL, 131.26 eq). The mixture was stirred at 25° C. for 1 h. LC-MS showed Reactant 1 was consumed completely. The reaction mixture was diluted with dichloromethane 5 mL, and aqueous sodium hydrogen carbonate was added to the mixture till pH=7. Then 15 mL water was added to the mixture, and extracted with the solution (dichloromethane/Methanol=10/1, V/V, 20 mL×3). The combined organic layers were washed with brine 40 mL, dried over anhydrous sodium sulfate, filtered and concentrated under reduced pressure to give a residue. The residue was purified by prep-HPLC (column: Welch Xtimate C18 150×25 mm×5 um; mobile phase: [water(formic acid)-acetonitrile]; gradient: 1%-30% B over 10 min). Compound 3-[5-[4-[1-[6-[[7-(7-fluoroimidazo[1,2-a]pyridin-3-yl)-3-oxo-isoindolin-4-yl]amino]-2-methyl-3-pyridyl]-4-piperidyl]-1-piperidyl]-3-methyl-2-oxo-benzimidazol-1-yl]piperidine-2,6-dione (22.1 mg, 0.02 mmol, 53% yield, 98% purity) was obtained as an off-white solid. MS (ESI) m/z: 797.5 [M+1]⁺; ¹HNMR (400 MHz, DMSO-d₆) δ 11.06 (s, 1H), 9.93 (s, 1H), 8.79 (s, 1H), 8.66 (d, J=8.4 Hz, 1H), 8.47-8.41 (m, 1H), 7.81 (s, 1H), 7.70 (d, J=8.4 Hz, 1H), 7.52 (dd, J=2.4, 10.0 Hz, 1H), 7.43 (d, J=8.4 Hz, 1H), 6.99-6.91 (m, 2H), 6.86-6.80 (m, 2H), 6.67-6.60 (m, 1H), 5.29 (dd, J=5.4, 12.8 Hz, 1H), 4.38 (s, 2H), 3.64 (d, J=11.2 Hz, 2H), 3.31 (s, 3H), 3.05 (d, J=10.4 Hz, 2H), 2.95-2.84 (m, 1H), 2.73-2.65 (m, 1H), 2.64-2.54 (m, 5H), 2.45 (s, 3H), 2.02-1.95 (m, 1H), 1.82 (d, J=11.6 Hz, 4H), 1.44-1.31 (m, 4H), 1.24 (d, J=2.0 Hz, 2H).

### Exemplification of Method K

### Example 301: Exemplary Synthesis of 3-(4-chloro-5-{4-[(4-{6-[(7-{7-fluoroimidazo[1,2-a]pyridin-3-yl}-3-oxo-2,3-dihydro-1H-isoindol-4-yl)amino]-2-methylpyridin-3-yl}-3-methyl-1H-pyrazol-1-yl)methyl]piperidin-1-yl}-1-oxo-2,3-dihydro-1H-isoindol-2-yl)piperidine-2,6-dione

### Step 1: Preparation of tert-butyl 7-[(5-bromo-6-methyl-2-pyridyl)amino]-4-(7-fluoroimidazo[1,2-a]pyridin-3-yl)-1-oxo-isoindoline-2-carboxylate

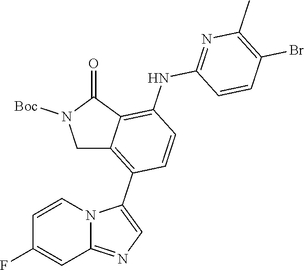 [SMILES: Cc1nc(Nc2ccc(-c3cnc4cc(F)ccn34)c3c2C(=O)N(C(=O)OC(C)(C)C)C3)ccc1Br]

A flask was charged with tert-butyl 7-amino-4-(7-fluoroimidazo[1,2-a]pyridin-3-yl)-1-oxo-isoindoline-2-carboxylate (1.47 g, 3.84 mmol, 1 eq), 3,6-dibromo-2-methyl-pyridine (965 mg, 3.84 mmol, 1 eq), tris(dibenzylideneacetone)dipalladium(0) (352 mg, 0.38 mmol, 0.1 eq), 4,5-bis(diphenylphosphino)-9,9-dimethylxanthene (334 mg, 0.58 mmol, 0.15 eq), potassium phosphate (2.45 g, 11.53 mmol, 3 eq) and dioxane (20 mL). The mixture was heated to 90° C. for 2 h under nitrogen. Reaction mixture was filtered and filtrate was concentrated. Crude product was kept in hand without purification. Tert-butyl 7-[(5-bromo-6-methyl-2-pyridyl)amino]-4-(7-fluoroimidazo[1,2-a]pyridin-3-yl)-1-oxo-isoindoline-2-carboxylate (2 g, crude) was obtained as a yellow solid.

### Step 2: Preparation of tert-butyl 4-[[3-methyl-4-(4,4,5,5-tetramethyl-1,3,2-dioxaborolan-2-yl)pyrazol-1-yl]methyl]piperidine-1-carboxylate

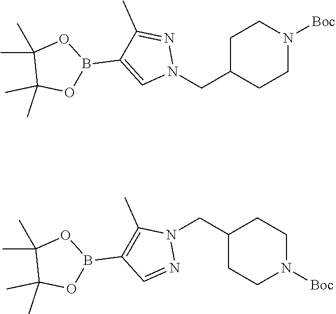 [SMILES: Cc1nn(CC2CCN(C(=O)OC(C)(C)C)CC2)cc1B1OC(C)(C)C(C)(C)O1 ; Cc1c(B2OC(C)(C)C(C)(C)O2)cnn1CC1CCN(C(=O)OC(C)(C)C)CC1]

To a solution of 3-methyl-4-(4,4,5,5-tetramethyl-1,3,2-dioxaborolan-2-yl)-1H-pyrazole (5 g, 24.03 mmol, 1 eq) and tert-butyl 4-(bromomethyl)piperidine-1-carboxylate (13 g, 46.73 mmol, 1.94 eq) in acetonitrile (40 mL) was added cesium carbonate (15.66 g, 48.06 mmol, 2 eq) and potassium iodide (798 mg, 4.81 mmol, 0.2 eq). The mixture was heated to 90° C. for 12 hr. LCMS showed reaction was complete. Reaction mixture was filtered and filtrate was concentrated in vacuum. Crude was purified by column (silica gel, ethyl acetate:petroleum ether=0:1-1:1). Tert-butyl 4-[[3-methyl-4-(4,4,5,5-tetramethyl-1,3,2-dioxaborolan-2-yl)pyrazol-1-yl]methyl]piperidine-1-carboxylate (8.9 g, 21.96 mmol, 91% yield) was obtained as a white solid. The product was a mixture of regio-isomers and ratio=1:1. ¹HNMR (400 MHz, CHLOROFORM-d) δ 7.69 (s, 0.5H), 7.52 (s, 0.5H), 3.88 (dd, J=4.8, 7.2 Hz, 2H), 2.64 (br t, J=12.0 Hz, 2H), 2.41 (s, 1.5H), 2.37 (s, 1.5H), 2.13-2.05 (m, 1H), 1.54 (d, J=12.4 Hz, 2H), 1.44 (s, 9H), 1.30 (s, 12H), 1.20-1.08 (m, 2H).

### Step 3: Preparation of tert-butyl 7-[[5-[1-[(1-tert-butoxycarbonyl-4-piperidyl)methyl]-3-methyl-pyrazol-4-yl]-6-methyl-2-pyridyl]amino]-4-(7-fluoroimidazo[1,2-a]pyridin-3-yl)-1-oxo-isoindoline-2-carboxylate

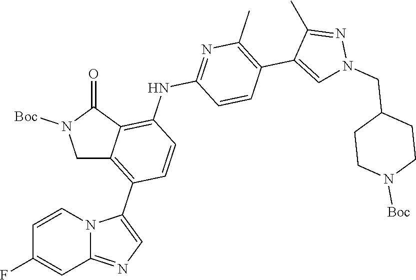 [SMILES: Cc1nc(Nc2ccc(-c3cnc4cc(F)ccn34)c3c2C(=O)N(C(=O)OC(C)(C)C)C3)ccc1-c1cn(CC2CCN(C(=O)OC(C)(C)C)CC2)nc1C]

A vial was charged with tert-butyl 7-[(5-bromo-6-methyl-2-pyridyl)amino]-4-(7-fluoroimidazo[1,2-a]pyridin-3-yl)-1-oxo-isoindoline-2-carboxylate (1.9 g, 1.82 mmol, 1 eq), tert-butyl 4-[[3-methyl-4-(4,4,5,5-tetramethyl-1,3,2-dioxaborolan-2-yl)pyrazol-1-yl]methyl]piperidine-1-carboxylate (887 mg, 2.19 mmol, 1.2 eq), [2-(2-aminophenyl) phenyl]-chloro-palladium;dicyclohexyl-[3-(2,4,6-triisopropylphenyl)phenyl]phosphane (143 mg, 0.18 mmol, 0.1 eq), potassium phosphate (774 mg, 3.65 mmol, 2 eq), dioxane (20 mL) and water (2 mL). The mixture was heated to 100° C. for 4 h under nitrogen protection. LCMS showed reaction was complete. Reaction mixture was filtered and filtrate was concentrated in vacuum. Crude was purified by column (dichloromethane:methanol=1: 0-80:1). Tert-butyl 7-[[5-[1-[(1-tert-butoxycarbonyl-4-piperidyl)methyl]-3-methyl-pyrazol-4-yl]-6-methyl-2-pyridyl]amino]-4-(7-fluoroimidazo[1,2-a]pyridin-3-yl)-1-oxo-isoindoline-2-carboxylate (0.66 g, 0.75 mmol, 41% yield, 85% purity) was obtained as a yellow solid. Tert-butyl 7-[[5-[1-[(1-tert-butoxycarbonyl-4-piperidyl)methyl]-3-methyl-pyrazol-4-yl]-6-methyl-2-pyridyl]amino]-4-(7-fluoroimidazo[1,2-a]pyridin-3-yl)-1-oxo-isoindoline-2-carboxylate (1.26 g, 0.89 mmol, 49% yield, 53% purity) was obtained as a yellow solid.

### Step 4: Preparation of tert-butyl 7-[[5-[1-[(1-tert-butoxycarbonyl-4-piperidyl)methyl]-5-methyl-pyrazol-4-yl]-6-methyl-2-pyridyl]amino]-4-(7-fluoroimidazo[1,2-a]pyridin-3-yl)-1-oxo-isoindoline-2-carboxylate & tert-butyl 7-[[5-[1-[(1-tert-butoxycarbonyl-4-piperidyl)methyl]-3-methyl-pyrazol-4-yl]-6-methyl-2-pyridyl]amino]-4-(7-fluoroimidazo[1,2-a]pyridin-3-yl)-1-oxo-isoindoline-2-carboxylate

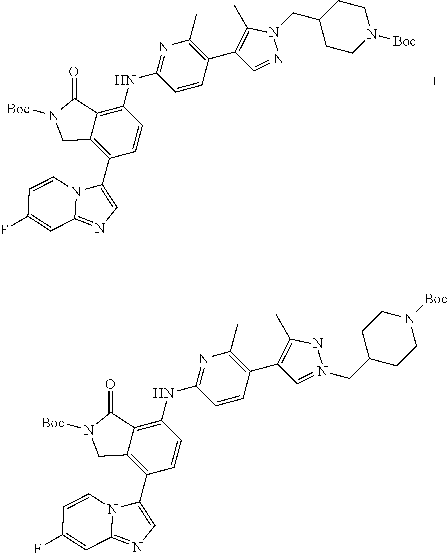

Tert-butyl 7-[[5-[1-[(1-tert-butoxycarbonyl-4-piperidyl)methyl]-3-methyl-pyrazol-4-yl]-6-methyl-2-pyridyl]amino]-4-(7-fluoroimidazo[1,2-a]pyridin-3-yl)-1-oxo-isoindoline-2-carboxylate (968 mg, 1.29 mmol, 1 eq) was separated by SFC column: DAICEL CHIRALCEL OD (250 mm*30 mm, 10 um); mobile phase: [CO2-ACN/EtOH(0.1% NH₃H2O)]; B %: 60%, isocratic elution mode. Tert-butyl 7-[[5-[1-[(1-tert-butoxycarbonyl-4-piperidyl)methyl]-5-methyl-pyrazol-4-yl]-6-methyl-2-pyridyl]amino]-4-(7-fluoroimidazo[1,2-a]pyridin-3-yl)-1-oxo-isoindoline-2-carboxylate (360 mg, 0.45 mmol, 69% yield, 93% purity) was obtained as a pale yellow solid.

¹H NMR (400 MHz, CHLOROFORM-d) δ 9.99 (s, 1H), 9.08 (d, J=8.4 Hz, 1H), 8.04 (t, J=6.4 Hz, 1H), 7.77-7.58 (m, 2H), 7.45 (s, 1H), 7.39 (d, J=8.4 Hz, 2H), 6.83-6.70 (m, 2H), 4.66 (s, 2H), 4.22-4.07 (m, 2H), 3.97 (d, J=7.2 Hz, 2H), 2.83-2.63 (m, 2H), 2.46 (s, 3H), 2.18 (s, 4H), 1.59 (s, 9H), 1.47 (s, 9H), 1.37-1.16 (m, 4H). Tert-butyl 7-[[5-[1-[(1-tert-butoxycarbonyl-4-piperidyl)methyl]-3-methyl-pyrazol-4-yl]-6-methyl-2-pyridyl]amino]-4-(7-fluoroimidazo[1,2-a]pyridin-3-yl)-1-oxo-isoindoline-2-carboxylate (500 mg, 0.54 mmol, 84% yield, 81% purity) was obtained as a pale yellow solid. ¹H NMR (400 MHz, CHLOROFORM-d) δ 10.00 (s, 1H), 9.09 (d, J=8.8 Hz, 1H), 8.06 (dd, J=5.6, 7.2 Hz, 1H), 7.75-7.64 (m, 2H), 7.43 (d, J=8.4 Hz, 2H), 7.29 (s, 1H), 6.83-6.73 (m, 2H), 4.67 (s, 2H), 4.20-4.14 (m, 2H), 3.98 (d, J=7.2 Hz, 2H), 2.80-2.65 (m, 2H), 2.48 (s, 3H), 2.21 (s, 3H), 2.16-2.10 (m, 1H), 1.61 (s, 9H), 1.48 (s, 9H), 1.44 (s, 1H), 1.29-1.13 (m, 3H).

### Step 5: Preparation of 4-(7-fluoroimidazo[1,2-a]pyridine-3-yl)-7-[[6-methyl-5-[5-methyl-1-(4-piperidylmethyl)pyrazol-4-yl]-2-pyridyl]amino]isoindolin-1-one

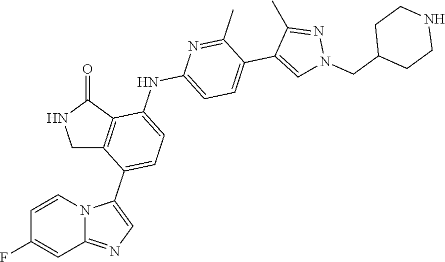 [SMILES: Cc1nc(Nc2ccc(-c3cnc4cc(F)ccn34)c3c2C(=O)NC3)ccc1-c1cn(CC2CCNCC2)nc1C]

To a solution of tert-butyl 7-[[5-[1-[(1-tert-butoxycarbonyl-4-piperidyl)methyl]-5-methyl-pyrazol-4-yl]-6-methyl-2-pyridyl]amino]-4-(7-fluoroimidazo[1,2-a]pyridin-3-yl)-1-oxo-isoindoline-2-carboxylate (360 mg, 0.48 mmol, 1 eq) in dichloromethane (5 mL) was added trifluoroacetic acid (273 mg, 2.40 mmol, 5 eq). The solution was stirred at 20° C. for 0.5 h. Solvent was removed in vacuum. Crude was purified by prep-HPLC column: C18 150×30 mm; mobile phase: [water-ACN(50% THF)]; gradient: 88%-38% B over 7 min. 4-(7-fluoroimidazo[1,2-a]pyridine-3-yl)-7-[[6-methyl-5-[5-methyl-1-(4-piperidylmethyl)pyrazol-4-yl]-2-pyridyl]amino]isoindolin-1-one (230 mg, 0.42 mmol, 87% yield, 100% purity) was obtained as a white solid. ¹H NMR (400 MHz, DMSO-d₆) δ 10.14 (s, 1H), 8.86 (s, 1H), 8.81 (d, J=8.4 Hz, 1H), 8.62 (d, J=9.2 Hz, 1H), 8.56 (dd, J=5.6, 7.2 Hz, 1H), 8.30 (d, J=10.4 Hz, 1H), 8.03 (s, 1H), 7.77 (d, J=8.4 Hz, 1H), 7.70 (dd, J=2.4, 9.6 Hz, 1H), 7.51-7.41 (m, 2H), 7.15 (dt, J=2.4, 7.2 Hz, 1H), 6.90 (d, J=8.4 Hz, 1H), 4.41 (s, 2H), 4.07-4.02 (m, 2H), 3.30 (d, J=12.4 Hz, 2H), 2.88 (q, J=11.6 Hz, 2H), 2.39 (s, 3H), 2.19 (s, 4H), 1.71 (d, J=12.8 Hz, 2H), 1.51-1.34 (m, 2H).

### Step 6: Preparation of 3-[4-chloro-5-[4-[[4-[6-[[7-(7-fluoroimidazo [1,2-a]pyridine-3-yl)-3-oxo-isoindolin-4-yl]amino]-2-methyl-3-pyridyl]-3-methyl-pyrazol-1-yl]methyl]-1-piperidyl]-1-oxo-isoindolin-2-yl]piperidine-2,6-dione

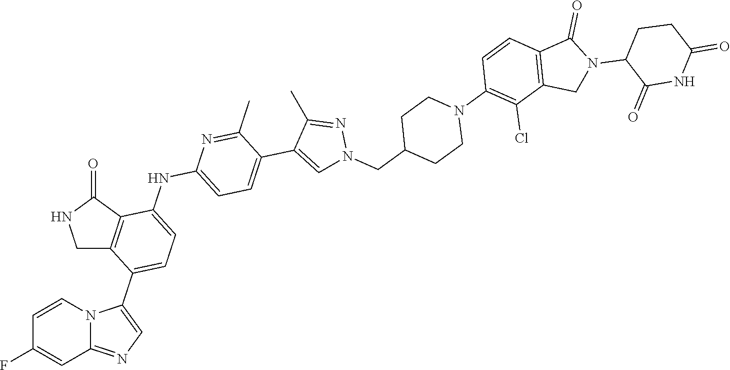 [SMILES: Cc1nc(Nc2ccc(-c3cnc4cc(F)ccn34)c3c2C(=O)NC3)ccc1-c1cn(CC2CCN(c3ccc4c(c3Cl)CN(C3CCC(=O)NC3=O)C4=O)CC2)nc1C]

A vial was charged with 4-(7-fluoroimidazo[1,2-a]pyridin-3-yl)-7-[[6-methyl-5-[3-methyl-1-(4-piperidylmethyl)pyrazol-4-yl]-2-pyridyl]amino]isoindolin-1-one (100 mg, 0.18 mmol, 1 eq), 3-(4-chloro-5-fluoro-1-oxo-isoindolin-2-yl)piperidine-2,6-dione (54 mg, 0.18 mmol, 1 eq), N,N-diisopropylethylamine (70 mg, 0.54 mmol, 3 eq) and dimethyl sulfoxide (1 mL). The solution was heated to 130° C. for 12 h. LCMS showed desired MS was detected. Reaction solution was filtered and filtrate was sent to prep-HPLC. Crude was purified by prep-HPLC column: C18 150×30 mm; mobile phase: [water(FA)-ACN]; gradient: 25%-55% B over 7 min. 3-[4-chloro-5-[4-[[4-[6-[[7-(7-fluoroimidazo[1,2-a]pyridin-3-yl)-3-oxo-isoindolin-4-yl]amino]-2-methyl-3-pyridyl]-3-methyl-pyrazol-1-yl]methyl]-1-piperidyl]-1-oxo-isoindolin-2-yl]piperidine-2,6-dione (15 mg, crude) was obtained as a pale yellow solid, which was further purified by prep-TLC (dichloromethane:methanol:ammonium hydroxide=10:1:0.05) to afford 3-[4-chloro-5-[4-[[4-[6-[[7-(7-fluoroimidazo[1,2-a]pyridin-3-yl)-3-oxo-isoindolin-4-yl]amino]-2-methyl-3-pyridyl]-3-methyl-pyrazol-1-yl]methyl]-1-piperidyl]-1-oxo-isoindolin-2-yl]piperidine-2,6-dione (8.1 mg, 0.0095 mmol, 52% yield, 97% purity) as a pale yellow solid. MS (ESI) m/z: 827.4 [M+1]⁺; ¹HNMR (400 MHz, DMSO-d6) δ 10.99 (s, 1H), 10.11 (s, 1H), 8.85 (s, 1H), 8.79 (d, J=8.4 Hz, 1H), 8.49-8.45 (m, 1H), 7.85 (s, 1H), 7.78-7.73 (m, 2H), 7.65 (d, J=8.0 Hz, 1H), 7.53 (dd, J=2.4, 10.0 Hz, 1H), 7.50 (d, J=8.4 Hz, 1H), 7.27 (d, J=8.4 Hz, 1H), 7.01-6.95 (m, 1H), 6.88 (d, J=8.4 Hz, 1H), 5.10 (dd, J=5.2, 13.2 Hz, 1H), 4.44-4.39 (m, 3H), 4.25 (d, J=17.2 Hz, 1H), 4.05 (d, J=7.2 Hz, 2H), 3.41 (d, J=6.0 Hz, 2H), 2.96-2.83 (m, 1H), 2.74 (t, J=10.8 Hz, 2H), 2.62-2.54 (m, 1H), 2.42 (s, 4H), 2.11 (s, 4H), 2.06-1.92 (m, 2H), 1.75-1.61 (m, 2H), 1.54-1.39 (m, 2H).

### Exemplification of Method L

### Example 302: Exemplary Synthesis of 3-[7-({5-[4-(2-{1-[1-(2,6-dioxopiperidin-3-yl)-3-methyl-2-oxo-2,3-dihydro-1H-1,3-benzodiazol-5-yl]piperidin-4-yl}acetyl)piperazin-1-yl]-6-methylpyridin-2-yl}amino)-1-oxo-2,3-dihydro-1H-isoindol-4-yl]imidazo[1,2-a]pyridine-7-carbonitrile

### Step 1: Preparation of 1-(2,6-bis(benzyloxy)pyridin-3-yl)-5-bromo-3-methyl-1,3-dihydro-2H-benzo[d]imidazol-2-one

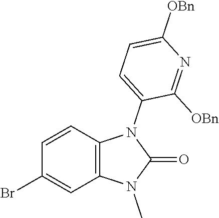 [SMILES: Cn1c(=O)n(-c2ccc(OCc3ccccc3)nc2OCc2ccccc2)c2ccc(Br)cc21]

To a solution of 6-bromo-3-(2,6-dibenzyloxy-3-pyridyl)-1H-benzimidazol-2-one (5 g, 9.95 mmol, 1 eq) in tetrahydrofuran (50 mL) was added sodium hydride (796 mg, 19.91 mmol, 60% purity, 2 eq) at 0° C. The mixture was stirred at 0° C. for 0.5 hr. Then iodomethane (2.1 g, 14.93 mmol, 1 mL, 1.5 eq) was added to the solution at 0° C. The mixture was warmed to 25° C. and stirred for 11.5 h. LC-MS showed Reactant 1 was consumed completely and one main peak with desired m/z was detected. The reaction mixture was partitioned between saturated ammonium chloride aqueous solution (100 mL) and ethyl acetate (200 mL). The organic phase was separated, washed with brine (50 mL×3), dried over anhydrous sodium sulfate, filtered and concentrated under reduced pressure to give a residue. The residue was purified by flash silica gel chromatography (ISCO®; 7 g SepaFlash® Silica Flash Column, Eluent of 0˜20% Ethyl acetate/Petroleum ethergradient @ 140 mL/min). Compound 5-bromo-1-(2,6-dibenzyloxy-3-pyridyl)-3-methyl-benzimidazol-2-one (4.7 g, 9.10 mmol, 91% yield) was obtained as an orange oil. MS (ESI) m/z: 516.0[M]; ¹H NMR (400 MHz, CHLOROFORM-d) δ 7.60 (d, J=8.4 Hz, 1H), 7.45-7.32 (m, 5H), 7.27-7.26 (m, 3H), 7.24-7.20 (m, 2H), 7.17-7.09 (m, 2H), 6.53 (t, J=7.6 Hz, 2H), 5.53-5.43 (m, 1H), 5.36 (s, 2H), 5.33-5.22 (m, 1H), 3.46 (s, 3H).

### Step 2: Preparation of tert-butyl 2-(1-(1-(2,6-bis(benzyloxy)pyridin-3-yl)-3-methyl-2-oxo-2,3-dihydro-1H-benzo[d]imidazol-5-yl) piperidin-4-yl) acetate

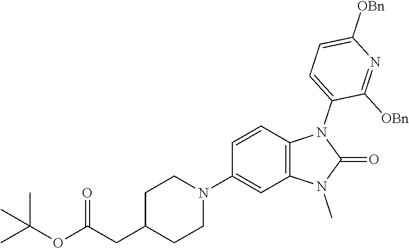 [SMILES: Cn1c(=O)n(-c2ccc(OCc3ccccc3)nc2OCc2ccccc2)c2ccc(N3CCC(CC(=O)OC(C)(C)C)CC3)cc21]

A mixture of 5-bromo-1-(2,6-dibenzyloxy-3-pyridyl)-3-methyl-benzimidazol-2-one (500 mg, 1 mmol, 1 eq), tert-butyl 2-(4-piperidyl) acetate (231 mg, 1.16 mmol, 1.2 eq), methanesulfonato(2-dicyclohexylphosphino-2,6-di-i-propoxy-1,1-biphenyl)(2-amino-1,1-biphenyl-2-yl)palladium(ii) (81 mg, 0.1 mmol, 0.1 eq) and cesium carbonate (631 mg, 1.94 mmol, 2 eq) in dioxane (10 mL) was degassed and purged with nitrogen for 3 times, and then the mixture was stirred at 100° C. for 12 hr under nitrogen atmosphere. LC-MS showed Reactant 1 was consumed completely and one main peak with desired m/z was detected. The reaction mixture was partitioned between water (50 mL) and ethyl acetate (150 mL). The organic phase was separated, washed with brine (30 mL×3), dried over anhydrous sodium sulfate, filtered and concentrated under reduced pressure to give a residue. The residue was purified by flash silica gel chromatography (ISCO®; 0.9 g SepaFlash® Silica Flash Column, Eluent of 0˜27% Ethyl acetate/Petroleum ethergradient @140 mL/min). Compound tert-butyl 2-[1-[1-(2,6-dibenzyloxy-3-pyridyl)-3-methyl-2-oxo-benzimidazol-5-yl]-4-piperidyl]acetate (456 mg, 0.7 mmol, 74% yield) was obtained as a yellow oil. MS (ESI) m/z: 635.3[M+1]⁺; ¹H NMR (400 MHz, CHLOROFORM-d) δ 7.60 (d, J=8.4 Hz, 1H), 7.45-7.33 (m, 5H), 7.24 (s, 5H), 6.69-6.62 (m, 2H), 6.61-6.56 (m, 1H), 6.50 (d, J=8.4 Hz, 1H), 5.51-5.41 (m, 1H), 5.37-5.26 (m, 3H), 3.55 (d, J=12.0 Hz, 2H), 3.45 (s, 3H), 2.74 (t, J=11.6 Hz, 2H), 2.23 (d, J=6.4 Hz, 2H), 1.94-1.79 (m, 3H), 1.48 (s, 10H).

### Step 3: Preparation of tert-butyl 2-(1-(1-(2,6-dioxopiperidin-3-yl)-3-methyl-2-oxo-2,3-dihydro-1H-benzo[d]imidazol-5-yl) piperidin-4-yl) acetate

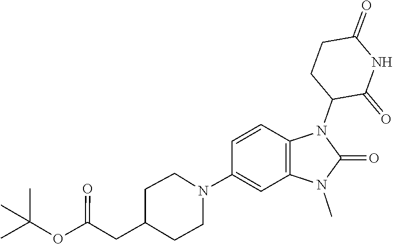 [SMILES: Cn1c(=O)n(C2CCC(=O)NC2=O)c2ccc(N3CCC(CC(=O)OC(C)(C)C)CC3)cc21]

To a solution of tert-butyl 2-[1-[1-(2,6-dibenzyloxy-3-pyridyl)-3-methyl-2-oxo-benzimidazol-5-yl]-4-piperidyl]acetate (456 mg, 0.7 mmol, 1 eq) in tetrahydrofuran (10 mL) was added palladium on activated carbon (76 mg, 0.07 mmol, 10% purity, 0.1 eq) and palladium hydroxide (101 mg, 0.07 mmol, 10% purity, 0.1 eq) under nitrogen atmosphere. The suspension was degassed and purged with hydrogen for 3 times. The mixture was stirred under hydrogen at 50° C. for 12 hr. LCMS showed Reactant 1 was consumed completely and one main peak with desired m/z was detected. The reaction mixture was concentrated under reduced pressure to remove solvent. The crude product was used into the next step without further purification. Compound tert-butyl 2-[1-[1-(2,6-dioxo-3-piperidyl)-3-methyl-2-oxo-benzimidazol-5-yl]-4-piperidyl]acetate (360 mg, crude) was obtained as a white solid. MS (ESI) m/z: 457.2[M+1]⁺.

### Step 4: Preparation of 2-(1-(1-(2,6-dioxopiperidin-3-yl)-3-methyl-2-oxo-2,3-dihydro-1H-benzo[d]imidazol-5-yl) piperidin-4-yl) acetic acid

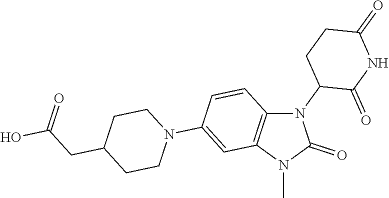 [SMILES: Cn1c(=O)n(C2CCC(=O)NC2=O)c2ccc(N3CCC(CC(=O)O)CC3)cc21]

To a solution of tert-butyl 2-[1-[1-(2,6-dioxo-3-piperidyl)-3-methyl-2-oxo-benzimidazol-5-yl]-4-piperidyl]acetate (360 mg, 0.8 mmol, 1 eq) in dichloromethane (3 mL) was added trifluoroacetic acid (2.3 g, 20.19 mmol, 25 eq). The mixture was stirred at 20° C. for 0.5 hr. LC-MS showed Reactant 1 was consumed completely and one main peak with desired m/z was detected. The reaction mixture was concentrated under reduced pressure to remove solvent. The residue was purified by prep-HPLC (column: Phenomenex luna C18 150*40 mm*15 um; mobile phase: [water (FA)-ACN]; gradient: 2%-32% B over 15 min). Compound 2-[1-[1-(2,6-dioxo-3-piperidyl)-3-methyl-2-oxo-benzimidazol-5-yl]-4-piperidyl]acetic acid (200 mg, 0.5 mmol, 63% yield) was obtained as a pink solid. MS (ESI) m/z: 401.1 [M+1]⁺; ¹H NMR (400 MHz, DMSO-d₆) δ 12.54-11.63 (m, 1H), 11.10 (s, 1H), 7.36-7.18 (m, 1H), 7.15-6.95 (m, 2H), 5.36 (dd, J=5.2, 12.8 Hz, 1H), 3.34 (s, 3H), 2.98-2.80 (m, 2H), 2.77-2.58 (m, 3H), 2.27 (d, J=6.4 Hz, 2H), 2.11-1.80 (m, 6H), 1.52 (d, J=10.0 Hz, 2H).

### Step 5: Preparation of 3-(7-((5-(4-(2-(1-(1-(2,6-dioxopiperidin-3-yl)-3-methyl-2-oxo-2,3-dihydro-1H-benzo[d]imidazol-5-yl) piperidin-4-yl) acetyl) piperazin-1-yl)-6-methylpyridin-2-yl) amino)-1-oxoisoindolin-4-yl) imidazo[1,2-a]pyridine-7-carbonitrile

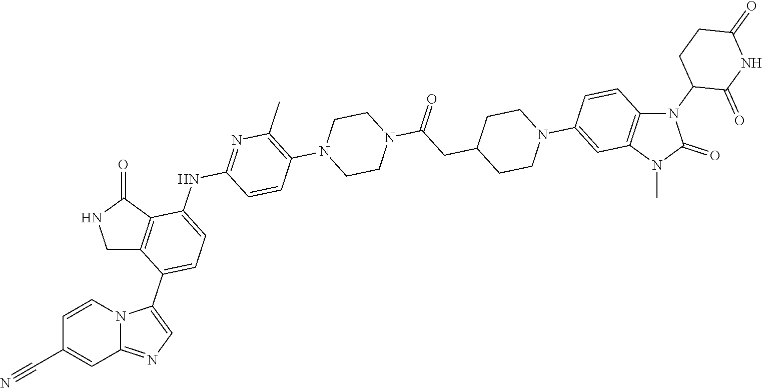 [SMILES: Cc1nc(Nc2ccc(-c3cnc4cc(C#N)ccn34)c3c2C(=O)NC3)ccc1N1CCN(C(=O)CC2CCN(c3ccc4c(c3)n(C)c(=O)n4C3CCC(=O)NC3=O)CC2)CC1]

To a solution of 3-[7-[(6-methyl-5-piperazin-1-yl-2-pyridyl) amino]-1-oxo-isoindolin-4-yl]imidazo[1,2-a]pyridine-7-carbonitrile (237 mg, 0.5 mmol, 1.2 eq) in N,N-dimethylformamide (5 mL) was added 2-[1-[1-(2,6-dioxo-3-piperidyl)-3-methyl-2-oxo-benzimidazol-5-yl]-4-piperidyl]acetic acid (170 mg, 0.4 mmol, 1 eq) and N-ethyl-N-propan-2-ylpropan-2-amine (165 mg, 1.27 mmol, 3 eq). Then O-(7-azabenzotriazol-1-yl)-N,N,N,N-tetramethyluronium hexafluorophosphate (194 mg, 0.5 mmol, 1.2 eq) was added to the solution at 0° C. The mixture was stirred at 25° C. for 2 hr. LC-MS showed Reactant 1 was consumed completely and one main peak with desired m/z was detected. The reaction mixture was partitioned between water (50 mL) and dichloromethane (100 mL). The organic phase was separated, dried over anhydrous sodium sulfate, filtered and concentrated under reduced pressure to give a residue. The residue was purified by prep-HPLC (column: Phenomenex luna C18 150*40 mm*15 um; mobile phase: [water (FA)-ACN]; gradient: 10%-40% B over 18 min). Compound 3-[7-[[5-[4-[2-[1-[1-(2,6-dioxo-3-piperidyl)-3-methyl-2-oxo-benzimidazol-5-yl]-4-piperidyl]acetyl]piperazin-1-yl]-6-methyl-2-pyridyl]amino]-1-oxo-isoindolin-4-yl]imidazo[1,2-a]pyridine-7-carbonitrile (175.3 mg, 0.2 mmol, 47% yield, 96.65% purity) was obtained as a yellow solid. MS (ESI) m/z: 847.4[M+1]⁺. ¹H NMR (400 MHz, DMSO-d₆) δ 11.06 (s, 1H), 9.99 (s, 1H), 8.82 (s, 1H), 8.69 (d, J=8.8 Hz, 1H), 8.54 (d, J=7.2 Hz, 1H), 8.43 (s, 1H), 8.14 (s, 1H), 7.76 (d, J=8.8 Hz, 1H), 7.47 (d, J=8.8 Hz, 1H), 7.20 (dd, J=1.6, 7.2 Hz, 1H), 7.00-6.91 (m, 1H), 6.91-6.77 (m, 2H), 6.65 (dd, J=3.2, 6.4 Hz, 1H), 5.35-5.18 (m, 1H), 4.41 (s, 2H), 3.70-3.54 (m, 6H), 3.31 (s, 6H), 2.94-2.74 (m, 5H), 2.73-2.55 (m, 4H), 2.46 (s, 1H), 2.38-2.31 (m, 2H), 2.04-1.94 (m, 1H), 1.92-1.73 (m, 3H), 1.45-1.30 (m, 2H).

### Exemplification of Method M

### Example 303: Exemplary Synthesis of 3-[6-(1-{6-[(7-{7-fluoroimidazo[1,2-a]pyridin-3-yl}-3-oxo-2,3-dihydro-1H-isoindol-4-yl)amino]-2-methylpyridin-3-yl}piperidin-4-yl)pyridin-3-yl]piperidine-2,6-dione

### Step 1: Preparation of tert-butyl 4-(5-bromo-2-pyridyl)-3,6-dihydro-2H-pyridine-1-carboxylate

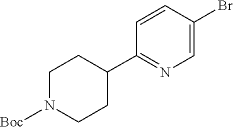 [SMILES: CC(C)(C)OC(=O)N1CCC(c2ccc(Br)cn2)CC1]

To a solution of 2,5-dibromopyridine (15 g, 63.32 mmol, 1 eq) and tert-butyl 4-(4,4,5,5-tetramethyl-1,3,2-dioxaborolan-2-yl)-3,6-dihydro-2H-pyridine-1-carboxylate (21.54 g, 69.65 mmol, 1.1 eq) in dioxane (150 mL) and water (15 mL) was added [1,1′-bis(diphenylphosphino)ferrocene]dichloropalladium(II) (2.32 g, 3.17 mmol, 0.05 eq) and sodium carbonate (20.13 g, 189.96 mmol, 3 eq). The mixture was degassed and purged with nitrogen for 3 times and stirred at 80° C. for 12 h under nitrogen atmosphere. LC-MS showed Reactant 1 was consumed completely and the desired mass was detected. The reaction mixture was filtered and the diluted with water 250 mL, then it was extracted with ethyl acetate (100 mL×3), the organic phase was combined and washed with saturated brine (100 mL), dried with anhydrous sodium sulfate, filtered and concentrated under reduced pressure to give a residue. The residue was purified by flash silica gel chromatography (ISCO®; 120 g SepaFlash® Silica Flash Column, Eluent of 0˜10% Ethyl acetate/Petroleum ethergradient @200 mL/min). Compound tert-butyl 4-(5-bromo-2-pyridyl)-3,6-dihydro-2H-pyridine-1-carboxylate (11.53 g, 33.99 mmol, 54% yield) was obtained as a yellow oil. MS (ESI) m/z: 282.9 [M−56+1]⁺. ¹HNMR (400 MHz, DMSO-d₆) δ 8.64 (d, J=2.4 Hz, 1H), 8.00 (dd, J=2.4, 8.4 Hz, 1H), 7.54 (d, J=8.4 Hz, 1H), 6.72 (s, 1H), 4.03 (s, 2H), 3.52 (t, J=5.2 Hz, 2H), 2.54 (s, 2H), 1.42 (s, 9H).

### Step 2: Preparation of tert-butyl 4-[5-(2,6-dibenzyloxy-3-pyridyl)-2-pyridyl]-3,6-dihydro-2H-pyridine-1-carboxylate

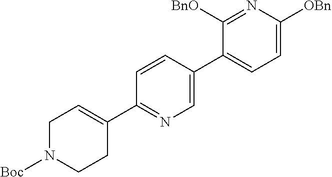 [SMILES: CC(C)(C)OC(=O)N1CC=C(c2ccc(-c3ccc(OCc4ccccc4)nc3OCc3ccccc3)cn2)CC1]

To a solution of tert-butyl 4-(5-bromo-2-pyridyl)-3,6-dihydro-2H-pyridine-1-carboxylate (11.53 g, 33.99 mmol, 1 eq) and (2,6-dibenzyloxy-3-pyridyl)boronic acid (13.67 g, 40.79 mmol, 1.2 eq) in dioxane (120 mL) was added potassium acetate (10.01 g, 101.97 mmol, 3 eq) and [1,1′-bis(diphenylphosphino)ferrocene]dichloropalladium(II) (1.24 g, 1.70 mmol, 0.05 eq). The mixture was degassed and purged with nitrogen for 3 times and stirred at 100° C. for 12 h under nitrogen atmosphere. LC-MS showed Reactant 1 was consumed completely and one main peak with desired mass was detected. The reaction mixture was filtered and the filtrate was concentrated under reduced pressure to give a residue. The residue was purified by flash silica gel chromatography (ISCO®; 120 g SepaFlash® Silica Flash Column, Eluent of 0˜15% Ethyl acetate/Petroleum ethergradient @200 mL/min). Compound tert-butyl 4-[5-(2,6-dibenzyloxy-3-pyridyl)-2-pyridyl]-3,6-dihydro-2H-pyridine-1-carboxylate (12.3 g, 22.38 mmol, 66% yield) was obtained as a yellow solid. MS (ESI) m/z: 550.4 [M+1]⁺. ¹HNMr (400 MHz, DMSO-d₆) δ 8.73 (d, J=2.0 Hz, 1H), 7.94 (dd, J=2.4, 8.4 Hz, 1H), 7.84 (d, J=8.0 Hz, 1H), 7.57 (d, J=8.4 Hz, 1H), 7.45-7.30 (m, 10H), 6.70 (s, 1H), 6.59 (d, J=8.0 Hz, 1H), 5.41 (d, J=11.6 Hz, 4H), 4.04 (d, J=6.8 Hz, 2H), 3.53 (t, J=5.6 Hz, 2H), 2.57 (s, 2H), 1.43 (s, 9H).

### Step 3: Preparation of tert-butyl 4-[5-(2,6-dioxo-3-piperidyl)-2-pyridyl]piperidine-1-carboxylate

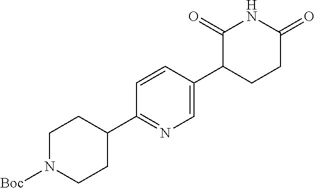 [SMILES: CC(C)(C)OC(=O)N1CCC(c2ccc(C3CCC(=O)NC3=O)cn2)CC1]

To a mixture of tert-butyl 4-[5-(2,6-dibenzyloxy-3-pyridyl)-2-pyridyl]-3,6-dihydro-2H-pyridine-1-carboxylate (12.26 g, 22.30 mmol, 1 eq) in dioxane (120 mL) was added Palladium hydroxide (2 g, 20% purity) under nitrogen. The mixture was stirred at 25° C. for 12 h under hydrogen. LCMS showed the starting material was consumed completely and desired compound was detected. The mixture was filtered and the filtrate was concentrated under reduced pressure to get a residue. The residue was purified by flash silica gel chromatography (ISCO®; 40 g SepaFlash® Silica Flash Column, Eluent of 0˜7% Methanol/Dichloromethane gradient @100 mL/min). Compound tert-butyl 4-[5-(2,6-dioxo-3-piperidyl)-2-pyridyl]piperidine-1-carboxylate (6.5 g, 16.71 mmol, 78% yield, 96% purity) was obtained as a white solid. MS (ESI) m/z: 374.1 [M+1]⁺. ¹HNMR (400 MHz, DMSO-d₆) δ 10.88 (s, 1H), 8.36 (d, J=2.0 Hz, 1H), 7.58 (dd, J=2.0, 8.0 Hz, 1H), 7.26 (d, J=8.0 Hz, 1H), 4.06 (d, J=11.6 Hz, 2H), 3.90 (dd, J=4.8, 12.4 Hz, 1H), 2.84 (ddd, J=3.6, 8.4, 11.6 Hz, 3H), 2.70 (s, 1H), 2.57-2.53 (m, 1H), 2.25 (dq, J=4.4, 12.8 Hz, 1H), 2.06-1.97 (m, 1H), 1.81 (d, J=12.0 Hz, 2H), 1.57 (dd, J=4.0, 12.4 Hz, 2H), 1.41 (s, 9H).

### Step 4: Preparation of 3-[6-(4-piperidyl)-3-pyridyl]piperidine-2,6-dione

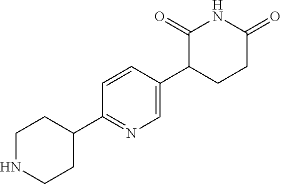 [SMILES: O=C1CCC(c2ccc(C3CCNCC3)nc2)C(=O)N1]

To a solution of tert-butyl 4-[5-(2,6-dioxo-3-piperidyl)-2-pyridyl]piperidine-1-carboxylate (6.5 g, 17.41 mmol, 1 eq) in dichloromethane (70 mL) was added trifluoroacetic acid (38.38 g, 336.55 mmol, 19.34 eq). The mixture was stirred at 25° C. for 1 h. TLC (dichloromethane:methanol=10:1) indicated Reactant 1 was consumed completely and one new spot formed. The reaction mixture was concentrated under reduced pressure to give a residue. The crude product was triturated with tert-butyl methyl ether 20 mL at 25° C. for 60 min. 3-[6-(4-piperidyl)-3-pyridyl]piperidine-2,6-dione (10.3 g, crude, 2 Trifluoroacetate) was obtained as a white solid.

¹HNMR (400 MHz, DMSO-d₆) δ 10.93 (s, 1H), 8.73 (d, J=8.4 Hz, 1H), 8.49 (d, J=2.0 Hz, 1H), 8.47-8.40 (m, 1H), 7.82 (dd, J=2.0, 8.0 Hz, 1H), 7.41 (d, J=8.0 Hz, 1H), 3.99 (dd, J=4.8, 12.8 Hz, 1H), 3.40 (d, J=12.4 Hz, 2H), 3.13-2.97 (m, 3H), 2.72 (s, 1H), 2.60-2.53 (m, 2H), 2.27 (s, 1H), 2.08-2.00 (m, 3H), 1.98-1.84 (m, 2H).

### Step 5: Preparation of 3-[6-[1-(2-methyl-6-nitro-3-pyridyl)-4-piperidyl]-3-pyridyl]piperidine-2,6-dione

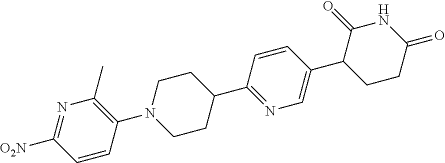 [SMILES: Cc1nc([N+](=O)[O-])ccc1N1CCC(c2ccc(C3CCC(=O)NC3=O)cn2)CC1]

To a solution of 3-[6-(4-piperidyl)-3-pyridyl]piperidine-2,6-dione (2 g, 3.99 mmol, 1 eq, 2 Trifluoroacetate) in N,N-dimethylformamide (20 mL) was added potassium carbonate (1.10 g, 7.98 mmol, 2 eq) and 3-fluoro-2-methyl-6-nitro-pyridine (750 mg, 4.80 mmol, 1.2 eq). The mixture was stirred at 40° C. for 12 h. LC-MS showed Reactant 1 was consumed completely and one main peak with desired mass was detected. The reaction mixture was filtered and poured into water 150 mL, then it was extracted with ethyl acetate (50 mL×3), the organic phase was combined and washed with saturated brine (50 mL×3), dried over anhydrous sodium sulfate, filtered and concentrated under reduced pressure to give a residue. The residue was purified by flash silica gel chromatography (ISCO @; 20 g SepaFlash® Silica Flash Column, Eluent of 0˜8% Methanol/Ethyl acetate gradient @100 mL/min). Compound 3-[6-[1-(2-methyl-6-nitro-3-pyridyl)-4-piperidyl]-3-pyridyl]piperidine-2,6-dione (1.3 g, 3.18 mmol, 80% yield) was obtained as a yellow solid. MS (ESI) m/z: 410.1 [M+1]. ¹HNMR (400 MHz, DMSO-d₆) δ 10.90 (s, 1H), 8.40 (d, J=2.0 Hz, 1H), 8.14 (d, J=8.8 Hz, 1H), 7.67-7.60 (m, 2H), 7.34 (d, J=8.0 Hz, 1H), 3.92 (dd, J=4.8, 12.4 Hz, 1H), 3.44 (d, J=12.0 Hz, 2H), 2.91 (dt, J=4.0, 11.2 Hz, 3H), 2.71 (s, 1H), 2.56 (d, J=4.0 Hz, 1H), 2.54 (s, 3H), 2.33-2.20 (m, 1H), 2.05-1.90 (m, 5H).

### Step 6: Preparation of 3-[6-[1-(6-amino-2-methyl-3-pyridyl)-4-piperidyl]-3-pyridyl]piperidine-2,6-dione

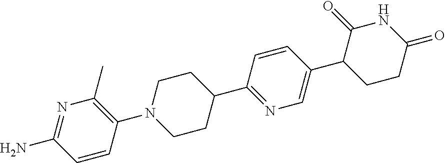 [SMILES: Cc1nc(N)ccc1N1CCC(c2ccc(C3CCC(=O)NC3=O)cn2)CC1]

To a solution of 3-[6-[1-(2-methyl-6-nitro-3-pyridyl)-4-piperidyl]-3-pyridyl]piperidine-2,6-dione (600 mg, 1.47 mmol, 1 eq) in ethanol (6 mL) and water (6 mL) was added ammonium chloride (784 mg, 14.65 mmol, 10 eq) and iron (246 mg, 4.40 mmol, 3 eq). The mixture was stirred at 50° C. for 2 h. LC-MS showed Reactant 1 was consumed completely and one main peak with desired mass was detected. The reaction was filtered and the filtrate was diluted with (dichloromethane:methanol=10:1, 50 mL), washed with water 20 mL, saturated brine 20 mL, dried over anhydrous sodium sulfate, filtered and concentrated under reduced pressure to give a residue. The crude product was triturated with tert-butyl methyl ether (5 mL) at 25° C. for 30 min. Compound 3-[6-[1-(6-amino-2-methyl-3-pyridyl)-4-piperidyl]-3-pyridyl]piperidine-2,6-dione (250 mg, 0.66 mmol, 45% yield) was obtained as a yellow solid. MS (ESI) m/z: 380.1 [M+1]⁺. ¹HNMR (400 MHz, DMSO-d₆) δ 10.89 (s, 1H), 8.38 (d, J=2.0 Hz, 1H), 7.60 (dd, J=2.0, 8.0 Hz, 1H), 7.34 (d, J=8.4 Hz, 1H), 7.30 (d, J=8.0 Hz, 1H), 6.35 (d, J=8.4 Hz, 1H), 5.97-5.56 (m, 2H), 3.91 (dd, J=4.8, 12.4 Hz, 1H), 2.99-2.93 (m, 3H), 2.76-2.65 (m, 5H), 2.56 (d, J=4.0 Hz, 1H), 2.29 (s, 3H), 2.04 (s, 1H), 1.91-1.88 (m, 3H).

### Step 7: Preparation of 3-[6-[1-[6-[[2-[(2,4-dimethoxyphenyl)methyl]-7-(7-fluoroimidazo[1,2-a]pyridin-3-yl)-3-oxo-isoindolin-4-yl]amino]-2-methyl-3-pyridyl]-4-piperidyl]-3-pyridyl]piperidine-2,6-dione

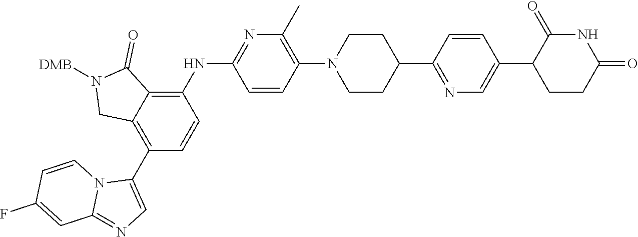

A mixture of 3-[6-[1-(6-amino-2-methyl-3-pyridyl)-4-piperidyl]-3-pyridyl]piperidine-2,6-dione (250 mg, 0.66 mmol, 1 eq), 7-chloro-2-[(2,4-dimethoxyphenyl)methyl]-4-(7-fluoroimidazo[1,2-a]pyridin-3-yl)isoindolin-1-one (330 mg, 0.73 mmol, 1.11 eq), cesium carbonate (429 mg, 1.32 mmol, 2 eq) and Methanesulfonato(2-dicyclohexylphosphino-2,6-di-i-propoxy-1,1-biphenyl)(2-amino-1,1-biphenyl-2-yl)palladium(II) (55 mg, 0.066 mmol, 0.1 eq) in dioxane (5 mL) was degassed and purged with nitrogen for 3 times, and then the mixture was stirred at 100° C. for 3 h under nitrogen atmosphere. LC-MS showed Reactant 1 was consumed completely and one main peak with desired mass was detected. The reaction mixture was filtered and the filtrate was concentrate under reduced pressure to give a residue. The residue was purified by flash silica gel chromatography (ISCO®; 12 g SepaFlash® Silica Flash Column, Eluent of 0˜8% Methanol/Dichloromethane gradient @100 mL/min). Compound 3-[6-[1-[6-[[2-[(2,4-dimethoxyphenyl)methyl]-7-(7-fluoroimidazo[1,2-a]pyridin-3-yl)-3-oxo-isoindolin-4-yl]amino]-2-methyl-3-pyridyl]-4-piperidyl]-3-pyridyl]piperidine-2,6-dione (300 mg, 0.38 mmol, 57% yield) was obtained as a yellow solid. MS (ESI) m/z: 795.2 [M+1]⁺.

### Step 8: Preparation of 3-[6-[1-[6-[[7-(7-fluoroimidazo[1,2-a]pyridin-3-yl)-3-oxo-isoindolin-4-yl]amino]-2-methyl-3-pyridyl]-4-piperidyl]-3-pyridyl]piperidine-2,6-dione

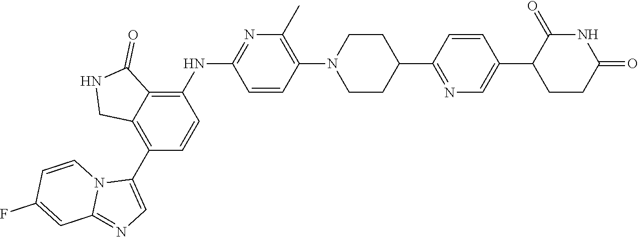 [SMILES: Cc1nc(Nc2ccc(-c3cnc4cc(F)ccn34)c3c2C(=O)NC3)ccc1N1CCC(c2ccc(C3CCC(=O)NC3=O)cn2)CC1]

To a solution of 3-[6-[1-[6-[[2-[(2,4-dimethoxyphenyl)methyl]-7-(7-fluoroimidazo[1,2-a]pyridin-3-yl)-3-oxo-isoindolin-4-yl]amino]-2-methyl-3-pyridyl]-4-piperidyl]-3-pyridyl]piperidine-2,6-dione (300 mg, 0.38 mmol, 1 eq) in trifluoroacetic acid (6 mL) was stirred at 60° C. for 3 h. LC-MS showed Reactant 1 was consumed completely and one main peak with desired mass was detected. The reaction mixture was concentrated under reduced pressure to give a residue and diluted with (dichloromethane:methanol=10:1, 20 mL) and water 10 mL, the it was adjusted the pH to 7-8 with aqueous sodium bicarbonate, separated and the aqueous phase was extracted with dichloromethane (20 mL×2), the organic phase was combined and washed with saturated brine (20 mL), dried with anhydrous sodium sulfate, filtered and concentrated under reduced pressure to give a residue. The residue was purified by prep-HPLC (column: Unisil 3-100 C18 Ultra 150*50 mm*3 um; mobile phase: [water(FA)-ACN]; gradient: 5%-35% B over 10 min). Compound 3-[6-[1-[6-[[7-(7-fluoroimidazo[1,2-a]pyridin-3-yl)-3-oxo-isoindolin-4-yl]amino]-2-methyl-3-pyridyl]-4-piperidyl]-3-pyridyl]piperidine-2,6-dione (59.9 mg, 0.089 mmol, 24% yield, 96% purity) was obtained as an off-white solid. MS (ESI) m/z: 645.4 [M+1]⁺. ¹HNMR (400 MHz, DMSO-d₆) δ 11.05 (s, 1H), 9.94 (s, 1H), 8.79 (s, 1H), 8.65 (d, J=8.4 Hz, 1H), 8.44 (dd, J=6.0, 7.2 Hz, 1H), 7.81 (s, 1H), 7.70 (d, J=8.8 Hz, 1H), 7.52 (dd, J=2.4, 10.0 Hz, 1H), 7.46 (d, J=8.4 Hz, 1H), 6.96 (dt, J=2.4, 7.6 Hz, 1H), 6.91 (d, J=8.8 Hz, 1H), 6.88 (d, J=2.0 Hz, 1H), 6.84 (d, J=8.8 Hz, 1H), 6.62 (dd, J=1.6, 8.8 Hz, 1H), 5.26 (dd, J=5.2, 12.8 Hz, 1H), 4.63-4.54 (m, 1H), 4.38 (s, 2H), 3.52 (d, J=12.0 Hz, 2H), 3.48-3.39 (m, 2H), 3.38-3.35 (m, 2H), 3.31-3.28 (m, 1H), 2.99-2.86 (m, 2H), 2.83 (s, 5H), 2.64-2.56 (m, 3H), 2.54 (s, 1H), 2.45 (s, 3H), 2.42 (s, 3H), 2.07 (s, 1H), 2.01-1.94 (m, 1H), 1.76 (d, J=12.0 Hz, 2H), 1.44 (d, J=6.8 Hz, 6H), 1.41-1.34 (m, 1H), 1.32-1.21 (m, 2H).

¹H NMR¹H NMR¹H NMRA compound of formula (I) where the variables are as defined above, can be prepared using standard reaction techniques known to one skilled in the art and following analogous procedures described as Methods A, B, or C. Generally, combining one intermediate containing a carbonyl group (aldehyde or ketone) with one amine containing intermediate under reductive amination conditions as described in Methods A, B, or C afford a compound of formula (I). Examples 5-294 and 304-457 were synthesized through analogous procedures described as Methods A, B, C, D, F, F, H, I, J, K, L, or M and the ¹H NMR data of the final compound is shown in the table below:

| Ex. | | Prep. |
|---|---|---|
| No. | NMR | Method |
| 5 | ¹H NMR (400 MHz, DMSO-d₆) δ 11.07 (s, 1H), 10.01 (s, 1H), 8.81 (s, | A |
| | 1H), 8.74 (d, J = 8.4 Hz, 1H), 8.44 (dd, J = 6.0, 7.2 Hz, 1H), 8.17 (s, | |
| | 2H), 7.82 (s, 1H), 7.72 (d, J = 8.4 Hz, 1H), 7.65 (dd, J = 1.6, 8.4 Hz, | |
| | 2H), 7.52 (dd, J = 2.4, 10.0 Hz, 1H), 7.32 (s, 1H), 7.26-7.22 (m, 1H), | |
| | 6.97 (d, J = 2.4 Hz, 1H), 6.91 (d, J = 8.8 Hz, 1H), 5.10-5.03 (m, 1H), | |
| | 4.38 (s, 2H), 4.06 (br d, J = 11.6Hz, 2H), 3.56 (s, 2H), 3.01-2.87 (m, | |
| | 7H), 2.61-2.54 (m, 5H), 2.24-2.18 (m, 7H), 2.04-1.96 (m, 3H), 1.83 | |
| | (d, J = 11.1 Hz, 2H), 1.68 (s, 2H), 1.24-1.14 (m, 2H) | |
| 6 | ¹H NMR (400 MHz, DMSO-d₆) δ 11.04 (s, 1H), 9.99 (s, 1H), 8.78 (s, | A |
| | 1H), 8.65 (d, J = 8.4 Hz, 1H), 8.39 (dd, J = 6.0, 7.2 Hz, 1H), 8.21-8.12 | |
| | (m, 2H), 7.78 (s, 1H), 7.69 (d, J = 8.4 Hz, 1H), 7.57 (d, J = 2.4 Hz, 1H), | |
| | 7.63-7.56 (m, 1H), 7.48 (dd, J = 2.4, 10.0 Hz, 1H), 7.28 (d, J = 1.6 Hz, | |
| | 1H), 7.20 (dd, J = 1.6, 8.8 Hz, 1H), 6.96-6.89 (m, 2H), 5.02 (dd, J = | |
| | 5.2, 12.8 Hz, 1H), 4.35 (s, 2H), 4.01 (d, J = 13.6 Hz, 2H), 2.98-2.88 | |
| | (m, 5H), 2.50 (s, 3H), 2.14 (d, J = 6.4 Hz, 2H), 2.02-1.94 (m, 3H), 1.78 | |
| | (d, J = 11.6 Hz, 3H), 1.74-1.67 (m, 3H), 1.66-1.57 (m, 2H) | |
| 7 | ¹H NMR (400 MHz, DMSO-d₆) δ 11.08 (s, 1H), 9.98 (s, 1H), 8.81 (s, | A |
| | 1H), 8.73 (d, J = 8.4 Hz, 1H), 8.45 (t, J = 6.8 Hz, 1H), 8.25 (s, 1H), 7.82 | |
| | (s, 1H), 7.72 (d, J = 8.8 Hz, 1H), 7.65 (d, J = 8.4 Hz, 1H), 7.59-7.49 | |
| | (m, 2H), 7.31 (s, 1H), 7.24 (d, J = 7.6 Hz, 1H), 7.00-6.92 (m, 1H), 6.84- | |
| | 6.79 (m, 1H), 5.07 (dd, J = 5.6, 12.8 Hz, 1H), 4.39 (s, 2H), 4.09-4.01 | |
| | (m, 2H), 3.04-2.81 (m, 7H), 2.71-2.55 (m, 3H), 2.19 (d, J = 7.2 Hz, | |
| | 2H), 2.06-1.98 (m, 3H), 1.86-1.78 (m, 3H), 1.71-1.60 (m, 4H), 1.22- | |
| | 1.11 (m, 2H) | |
| 8 | ¹H NMR (400 MHz, DMSO-d₆) δ 10.97 (s, 1H), 10.03 (s, 1H), 8.82 (s, | A |
| | 1H), 8.68 (d, J = 8.4 Hz, 1H), 8.45-8.41 (m, 1H), 8.24 (s, 1H), 8.20 (d, | |
| | J = 2.0 Hz, 1H), 7.82 (s, 1H), 7.72 (d, J = 8.4 Hz, 1H), 7.63 (dd, J = 2.0, | |
| | 8.4 Hz, 1H), 7.52 (dd, J = 2.4, 10.0 Hz, 1H), 7.41 (d, J = 8.4 Hz, 1H), | |
| | 7.25 (dd, J = 1.6, 8.4 Hz, 1H), 7.16 (d, J = 1.6 Hz, 1H), 6.96 (d, J = 8.4 | |
| | Hz, 2H), 5.09 (dd, J = 5.2, 13.2 Hz, 1H), 4.39-4.31 (m, 3H), 4.22- | |
| | 4.17 (m, 1H), 3.76 (d, J = 11.6 Hz, 3H), 2.97 (d, J = 10.8 Hz, 2H), 2.72 | |
| | (t, J = 11.6 Hz, 2H), 2.40-2.33 (m, 1H), 2.20 (d, J = 6.4 Hz, 2H), 2.05- | |
| | 1.95 (m, 4H), 1.85-1.67 (m, 8H), 1.29-1.21 (m, 2H) | |
| 9 | ¹H NMR (400 MHz, DMSO-d₆) δ 10.99 (s, 1H), 9.99 (s, 1H), 8.81 (s, | A |
| | 1H), 8.73 (d, J = 8.4 Hz, 1H), 8.47-8.42 (m, 1H), 8.15 (s, 1H), 7.82 (s, | |
| | 1H), 7.72 (d, J = 8.4 Hz, 1H), 7.65 (d, J = 8.0 Hz, 1H), 7.58-7.49 (m, | |
| | 2H), 7.27 (d, J = 8.4 Hz, 1H), 7.00-6.93 (m, 1H), 6.82 (d, J = 8.4 Hz, | |
| | 1H), 5.10 (dd, J = 4.8, 13.2 Hz, 1H), 4.45-4.36 (m, 3H), 4.25 (d, J = | |
| | 17.6 Hz, 1H), 3.41 (s, 2H), 3.04 (d, J = 10.4 Hz, 2H), 2.91 (s, 1H), 2.80- | |
| | 2.65 (m, 3H), 2.61 (s, 1H), 2.51 (s, 3H), 2.47 (s, 1H), 2.32 (d, J = 6.0 | |
| | Hz, 2H), 2.13 (t, J = 10.0 Hz, 2H), 1.99 (s, 1H), 1.87 (d, J = 10.8 Hz, | |
| | 2H), 1.80-1.60 (m, 5H), 1.34 (d, J = 11.2 Hz, 2H) | |
| 10 | ¹H NMR (400 MHz, DMSO-d₆) δ 11.04 (s, 1H), 9.96 (s, 1H), 8.80 (s, | A |
| | 1H), 8.72 (d, J = 8.8 Hz, 1H), 8.44 (dd, J = 5.6, 7.2 Hz, 1H), 7.80 (s, | |
| | 1H), 7.72 (d, J = 8.8 Hz, 1H), 7.56-7.48 (m, 2H), 7.00-6.92 (m, 2H), | |
| | 6.80 (d, J = 8.4 Hz, 1H), 6.64 (s, 1H), 5.00 (dd, J = 5.6, 12.8 Hz, 1H), | |
| | 4.36 (s, 2H), 4.1-4.04 (m, 2H), 3.92 (s, 3H), 3.04-2.80 (m, 6H), 2.64- | |
| | 2.52 (m, 4H), 2.48-2.40 (m, 2H), 2.24-2.12 (m, 2H), 2.08-1.92 (m, | |
| | 3H), 1.86-1.80 (m, 2H), 1.76-1.56 (m, 4H), 1.24-1.12 (m, 2H) | |
| 11 | ¹H NMR (400 MHz, DMSO-d₆) δ 11.00 (s, 1H), 10.03 (s, 1H), 8.83 (s, | A |
| | 1H), 8.69 (d, J = 8.4 Hz, 1H), 8.46-8.40 (m, 1H), 8.20 (d, J = 2.0 Hz, | |
| | 1H), 7.82 (s, 1H), 7.72 (d, J = 8.4 Hz, 1H), 7.67-7.60 (m, 2H), 7.52 | |
| | (dd, J = 2.4, 10.0 Hz, 1H), 7.27 (d, J = 8.4 Hz, 1H), 7.00-6.94 (m, 2H), | |
| | 5.10 (dd, J = 5.2, 13.2 Hz, 1H), 4.45-4.37 (m, 3H), 4.25 (d, J = 17.6 | |
| | Hz, 1H), 3.06-2.85 (m, 4H), 2.74 (t, J = 11.2 Hz, 2H), 2.64-2.53 (m, | |
| | 2H), 2.49-2.41 (m, 2H), 2.26 (d, J = 6.4 Hz, 2H), 2.07-1.97 (m, 3H), | |
| | 1.90-1.82 (m, 2H), 1.80-1.65 (m, 5H), 1.37-1.26 (m, 2H) | |
| 12 | ¹H NMR (400 MHz, DMSO-d₆) δ 11.08-10.96 (m, 1H), 10.08-9.96 | A |
| | (m, 1H), 8.80 (s, 1H), 8.64 (d, J = 8.4 Hz, 1H), 8.48-8.40 (m, 1H), 8.20 | |
| | (s, 1H), 7.80 (s, 1H), 7.72 (d, J = 8.4 Hz, 1H), 7.64-7.60 (m, 1H), 7.52 | |
| | (dd, J = 2.4, 10.4 Hz, 1H), 7.02-6.92 (m, 3H), 6.64 (s, 1H), 5.02 (dd, J = | |
| | 5.2, 12.8 Hz, 1H), 4.48-4.28 (m, 2H), 4.08 (d, J = 12.8 Hz, 2H), 3.92 | |
| | (s, 3H), 3.04-2.80 (m, 6H), 2.44-2.36 (m, 4H), 2.20 (d, J = 5.6 Hz, | |
| | 2H), 2.04-1.96 (m, 3H), 1.84-1.76 (m, 4H), 1.72-1.60 (m, 2H), | |
| | 1.24-1.12 (m, 2H) | |
| 13 | ¹H NMR (400 MHz, DMSO-d₆) δ 10.96 (s, 1H), 9.98 (s, 1H), 8.79 (s, | A |
| | 1H), 8.72 (d, J = 8.4 Hz, 1H), 8.43 (dd, J = 6.0, 7.2 Hz, 1H), 7.81 (s, | |
| | 1H), 7.71 (d, J = 8.4 Hz, 1H), 7.56 (d, J = 8.4 Hz, 1H), 7.51 (dd, J = 2.4, | |
| | 10.0 Hz, 1H), 7.42 (d, J = 8.4 Hz, 1H), 7.26 (dd, J = 2.0, 8.8 Hz, 1H), | |
| | 7.17 (d, J = 2.0 Hz, 1H), 6.96 (dt, J = 2.4, 7.6 Hz, 1H), 6.83 (d, J = 8.4 | |
| | Hz, 1H), 5.09 (dd, J = 5.2, 13.2 Hz, 1H), 4.38 (s, 2H), 4.33 (d, J = 16.8 | |
| | Hz, 1H), 4.23-4.18 (m, 1H), 3.79-3.75 (m, 2H), 2.91-2.86 (m, 2H), | |
| | 2.72 (d, J = 11.2 Hz, 5H), 2.62-2.57 (m, 2H), 2.38 (dd, J = 4.4, 12.8 | |
| | Hz, 3H), 2.27-2.16 (m, 3H), 2.04-1.93 (m, 2H), 1.87-1.81 (m, 2H), | |
| | 1.76-1.68 (m, 4H), 1.31-1.20 (m, 2H) | |
| 14 | ¹H NMR (400 MHz, DMSO-d₆) δ 11.04 (s, 1H), 10.02 (s, 1H), 8.83- | A |
| | 8.77 (m, 2H), 8.48-8.41 (m, 1H), 7.82 (s, 1H), 7.74 (d, J = 8.4 Hz, 1H), | |
| | 7.57 (d, J = 8.4 Hz, 1H), 7.52 (dd, J = 2.4, 10.0 Hz, 1H), 7.00-6.92 (m, | |
| | 2H), 6.77 (d, J = 8.4 Hz, 1H), 6.64 (s, 1H), 5.00 (dd, J = 5.6, 13.2 Hz, | |
| | 1H), 4.38 (s, 2H), 4.11-4.05 (m, 2H), 3.92 (s, 3H), 3.06-2.90 (m, 5H), | |
| | 2.89-2.80 (m, 1H), 2.77-2.70 (m, 1H), 2.64-2.58 (m, 2H), 2.20 (d, J = | |
| | 5.6 Hz, 2H), 2.09-2.01 (m, 2H), 2.00-1.93 (m, 1H), 1.89-1.80 (m, | |
| | 3H), 1.71-1.62 (m, 4H), 1.29 (d, J = 6.4 Hz, 6H), 1.21-1.13 (m, 2H) | |
| 15 | ¹H NMR (400 MHz, DMSO-d₆) δ 11.08 (s, 1H), 10.00 (s, 1H), 8.80 (s, | A |
| | 1H), 8.72 (d, J = 8.8 Hz, 1H), 8.40 (dd, J = 6.2, 7.2 Hz, 1H), 8.20 (s, | |
| | 1H), 7.60 (s, 1H), 7.68 (dd, J = 8.8, 14.4 Hz, 2H), 7.60-7.48 (m, 2H), | |
| | 7.32 (d, J = 1.6 Hz, 1H), 7.24 (dd, J = 2.0, 8.8 Hz, 1H), 7.00-6.92 (m, | |
| | 2H), 5.06 (dd, J = 5.4, 12.8 Hz, 1H), 4.36 (s, 2H), 4.04 (d, J = 12.4 Hz, | |
| | 2H), 3.56 (s, 3H), 3.44 (q, J = 7.2 Hz, 2H), 3.08-2.76 (m, 8H), 2.60 (d, | |
| | J = 2.4 Hz, 1H), 2.32 (s, 6H), 2.20 (d, J = 6.4 Hz, 2H), 2.08-1.96 (m, | |
| | 1H), 1.92-1.76 (m, 3H), 1.24-1.06 (m, 2H), 1.02 (t, J = 6.8 Hz, 1H) | |
| 16 | ¹H NMR (400 MHz, DMSO-d₆) δ 11.05 (s, 1H), 10.00 (s, 1H), 8.83- | A |
| | 8.75 (m, 2H), 8.47-8.42 (m, 1H), 8.17 (s, 1H), 7.82 (s, 1H), 7.72 (d, J = | |
| | 8.4 Hz, 1H), 7.57 (d, J = 8.4 Hz, 1H), 7.52 (dd, J = 2.4, 10.0 Hz, 1H), | |
| | 6.99-6.92 (m, 2H), 6.80 (d, J = 8.4 Hz, 1H), 6.64 (s, 1H), 5.00 (dd, J = | |
| | 5.2, 12.8 Hz, 1H), 4.38 (s, 2H), 4.08 (d, J = 13.2 Hz, 2H), 3.92 (s, 2H), | |
| | 3.96-3.89 (m, 1H), 3.04-2.95 (m, 4H), 2.90-2.77 (m, 3H), 2.61- | |
| | 2.56 (m, 2H), 2.21 (d, J = 6.4 Hz, 2H), 2.10-1.92 (m, 4H), 1.83 (d, J = | |
| | 11.2 Hz, 2H), 1.67 (d, J = 1.2 Hz, 4H), 1.30 (t, J = 7.6 Hz, 3H), 1.25- | |
| | 1.09 (m, 3H) | |
| 17 | ¹H NMR (400 MHz, DMSO-d₆) δ 10.95 (s, 1H), 9.98 (s, 1H), 8.81 (s, | A |
| | 1H), 8.73 (d, J = 8.4 Hz, 1H), 8.48-8.41 (m, 1H), 8.17 (s, 1H), 7.82 (s, | |
| | 1H), 7.73 (t, J = 8.8 Hz, 2H), 7.58-7.50 (m, 2H), 6.97 (dt, J = 2.8, 7.6 | |
| | Hz, 1H), 6.90 (d, J = 9.2 Hz, 1H), 6.82 (d, J = 8.4 Hz, 1H), 5.07 (dd, J = | |
| | 5.2, 13.2 Hz, 1H), 4.45 (d, J = 12.8 Hz, 2H), 4.39 (s, 2H), 4.27 (d, J = | |
| | 17.2 Hz, 1H), 4.09 (d, J = 17.2 Hz, 1H), 3.02-2.86 (m, 6H), 2.69-2.60 | |
| | (m, 2H), 2.56 (s, 2H), 2.40-2.32 (m, 1H), 2.21 (d, J = 6.0 Hz, 2H), 2.05 | |
| | (t, J = 10.8 Hz, 2H), 2.00-1.87 (m, 2H), 1.82 (d, J = 13.2 Hz, 2H), | |
| | 1.74-1.60 (m, 4H), 1.17-1.01 (m, 2H) | |
| 18 | ¹H NMR (400 MHz, DMSO-d₆) δ 11.04 (s, 1H), 10.02 (s, 1H), 8.81 (s, | A |
| | 1H), 8.74 (d, J = 8.8 Hz, 1H), 8.43 (dd, J = 6.0, 7.2 Hz, 1H), 8.16 (s, | |
| | 1H), 7.82 (s, 1H), 7.71 (d, J = 8.4 Hz, 1H), 7.66 (d, J = 8.4 Hz, 1H), | |
| | 7.52 (dd, J = 2.4, 10.0 Hz, 1H), 7.07-6.86 (m, 3H), 6.64 (s, 1H), 5.24- | |
| | 5.05 (m, 1H), 5.00 (dd, J = 5.2, 12.8 Hz, 1H), 4.62 (s, 2H), 4.39 (s, 2H), | |
| | 4.08 ( d, J = 13.2 Hz, 2H), 3.92 (s, 3H), 3.04-2.82 (m, 7H), 2.60-2.53 | |
| | (m, 1H), 2.22 ( d, J = 6.0 Hz, 2H), 2.07-1.94 (m, 3H), 1.91-1.77 (m, | |
| | 3H), 1.75-1.59 (m, 4H), 1.24-1.12 (m, 2H) | |
| 19 | ¹H NMR (400 MHz, DMSO-d₆) δ 10.80 (s, 1H), 9.93 (s, 1H), 8.79 (s, | A |
| | 1H), 8.65 (d, J = 8.4 Hz, 1H), 8.44 (t, J = 6.4 Hz, 1H), 8.14 (s, 1H), 7.93 | |
| | (d, J = 2.0 Hz, 1H), 7.81 (s, 1H), 7.70 (d, J = 8.4 Hz, 1H), 7.52 (dd, J = | |
| | 2.4, 10.0 Hz, 1H), 7.47 (d, J = 8.4 Hz, 1H), 7.36 (dd, J = 2.0, 8.4 Hz, | |
| | 1H), 6.96 (dt, J = 2.4, 7.2 Hz, 1H), 6.84 (d, J = 8.8 Hz, 1H), 6.79 (d, J = | |
| | 8.8 Hz, 1H), 4.38 (s, 2H), 4.26 (d, J = 12.4 Hz, 2H), 3.72 (dd, J = 4.8, | |
| | 12.0 Hz, 2H), 2.84 (s, 4H), 2.76 (d, J = 11.6 Hz, 4H), 2.67 (d, J = 3.6 | |
| | Hz, 1H), 2.46 (s, 3H), 2.22-2.22 (m, 1H), 2.28-2.09 (m, 3H), 2.04- | |
| | 1.91 (m, 2H), 1.79 (d, J = 10.4 Hz, 3H), 1.16-1.06 (m, 2H) | |
| 20 | ¹H NMR (400 MHz, DMSO-d₆) δ 10.96 (s, 1H), 9.98 (s, 1H), 8.80 (s, | A |
| | 1H), 8.73 (d, J = 8.4 Hz, 1H), 8.47-8.42 (m, 1H), 8.15 (s, 1H), 7.82 (s, | |
| | 1H), 7.71 (d, J = 8.8 Hz, 1H), 7.58-7.49 (m, 2H), 6.96 (dt, J = 2.8, 7.6 | |
| | Hz, 1H), 6.90 (d, J = 7.2 Hz, 1H), 6.82 (d, J = 8.4 Hz, 1H), 5.02 (dd, J = | |
| | 5.2, 13.2 Hz, 1H), 4.44-4.34 (m, 3H), 4.24 (d, J = 16.8 Hz, 1H), 3.57- | |
| | 3.44 (m, 3H), 3.09-2.92 (m, 3H), 2.92-2.84 (m, 1H), 2.81-2.72 (m, | |
| | 2H), 2.71-2.56 (m, 3H), 2.54 (s, 3H), 2.41 (dd, J = 4.4, 13.2 Hz, 1H), | |
| | 2.27 (d, J = 6.8 Hz, 2H), 2.09 (t, J = 10.0 Hz, 2H), 2.00-1.92 (m, 1H), | |
| | 1.84 (d, J = 12.0 Hz, 2H), 1.76-1.59 (m, 5H), 1.36-1.22 (m, 2H) | |
| 21 | ¹H NMR (400 MHz, DMSO-d₆) δ 10.98 (s, 1H), 10.01 (s, 1H), 8.82- | A |
| | 8.74 (m, 2H), 8.47-8.41 (m, 1H), 8.14 (s, 1H), 7.82 (s, 1H), 7.72 (d, J = | |
| | 8.4 Hz, 1H), 7.65 (d, J = 8.0 Hz, 1H), 7.57 (d, J = 8.4 Hz, 1H), 7.51 | |
| | (dd, J = 2.4, 10.2 Hz, 1H), 7.27 (d, J = 8.4 Hz, 1H), 6.96 (dt, J = 2.8, 7.6 | |
| | Hz, 1H), 6.81 (d, J = 8.4 Hz, 1H), 5.10 (dd, J = 4.8, 13.2 Hz, 1H), 4.45- | |
| | 4.36 (m, 3H), 4.26 (d, J = 17.2 Hz, 1H), 3.83-3.76 (m, 1H), 3.06 (d, J = | |
| | 10.4 Hz, 2H), 2.99-2.94 (m, 1H), 2.92-2.82 (m, 4H), 2.79-2.71 (m, | |
| | 4H), 2.59 (d, J = 17.6 Hz, 2H), 2.39-2.32 (m, 2H), 2.23-2.14 (m, 2H), | |
| | 2.02-1.97 (m, 1H), 1.88 (d, J = 10.8 Hz, 2H), 1.70 (s, 4H), 1.39-1.35 | |
| | (m, 1H), 1.31 (t, J = 7.2 Hz, 3H) | |
| 22 | ¹H NMR (400 MHz, DMSO-d₆) δ 11.03 (s, 1H), 10.02-9.98 (m, 1H), | |
| | 8.80 (s, 1H), 8.73 (d, J = 8.4 Hz, 1H), 8.46-8.41 (m, 1H), 8.17 (s, 1H), | |
| | 7.81 (s, 1H), 7.71 (d, J = 8.4 Hz, 1H), 7.59 (d, J = 8.4 Hz, 1H), 7.51 (dd, | |
| | J = 2.4, 10.1 Hz, 1H), 6.98-6.93 (m, 2H), 6.80 (d, J = 8.4 Hz, 1H), 6.64 | |
| | (s, 1H), 5.03-4.97 (m, 1H), 4.39-4.36 (m, 2H), 4.12-4.05 (m, 2H), | |
| | 3.92 (s, 3H), 3.05-2.95 (m, 5H), 2.93-2.78 (m, 2H), 2.69-2.67 (m, | |
| | 2H), 2.23-2.16 (m, 3H), 2.11-1.90 (m, 4H), 1.89-1.80 (m, 3H), 1.70- | |
| | 1.61 (m, 4H), 1.21-1.13 (m, 2H), 0.97 (d, J = 6.4 Hz, 6H) | |
| 23 | ¹H NMR (400 MHz, DMSO-d₆) δ 11.03 (s, 1H), 10.05 (s, 1H), 8.81 (s, | A |
| | 1H), 8.75 (d, J = 8.4 Hz, 1H), 8.44 (t, J = 6.8 Hz, 1H), 7.82 (s, 1H), 7.72 | |
| | (d, J = 8.4 Hz, 1H), 7.69 (d, J = 8.4 Hz, 1H), 7.51 (dd, J = 2.4, 10.0 Hz, | |
| | 1H), 6.99-6.92 (m, 3H), 6.64 (s, 1H), 4.99 (dd, J = 5.2, 12.9 Hz, 1H), | |
| | 4.56 (s, 2H), 4.39 (s, 2H), 4.07 (d, J = 13.2 Hz, 2H), 3.93 (s, 3H), 3.05- | |
| | 2.93 (m, 5H), 2.93-2.72 (m, 4H), 2.61-2.57 (m, 2H), 2.20 (d, J = 5.6 | |
| | Hz, 2H), 2.05-1.94 (m, 3H), 1.83 (d, J = 12.4 Hz, 3H), 1.67 (s, 4H), | |
| | 1.18 (d, J = 12.4 Hz, 2H) | |
| 24 | ¹H NMR (400 MHz, DMSO-d₆) δ 10.99 (s, 1H), 10.36 (s, 1H), 8.92 (s, | A |
| | 1H), 8.75 (d, J = 8.4 Hz, 1H), 8.50-8.44 (m, 1H), 7.99 (d, J = 8.8 Hz, | |
| | 1H), 7.84 (s, 1H), 7.79 (d, J = 8.8 Hz, 1H), 7.69-7.62 (m, 1H), 7.52 | |
| | (dd, J = 2.4, 9.6 Hz, 1H), 7.27 (dd, J = 4.8, 8.4 Hz, 2H), 6.96 (dt, J = | |
| | 2.4, 7.6 Hz, 1H), 5.10 (dd, J = 4.8, 13.6 Hz, 1H), 4.46-4.35 (m, 3H), | |
| | 4.25 (d, J = 17.2 Hz, 1H), 3.01 (d, J = 10.8 Hz, 2H), 2.93-2.85 (m, | |
| | 1H), 2.82-2.69 (m, 3H), 2.64-2.53 (m, 3H), 2.26 (d, J = 6.4 Hz, 2H), | |
| | 2.05-1.94 (m, 3H), 1.91-1.60 (m, 8H), 1.33 (d, J = 10.0 Hz, 2H) | |
| 25 | ¹H NMR (400 MHz, DMSO-d₆) δ 10.98 (s, 1H), 9.94 (s, 1H), 8.79 (s, | A |
| | 1H), 8.66 (d, J = 8.4 Hz, 1H), 8.46-8.42 (m, 1H), 7.81 (s, 1H), 7.70 (d, | |
| | J = 8.4 Hz, 1H), 7.65 (d, J = 8.0 Hz, 1H), 7.52 (dd, J = 2.4, 10.0 Hz, | |
| | 1H), 7.47 (d, J = 8.8 Hz, 1H), 7.27 (d, J = 8.4 Hz, 1H), 6.96 (dt, J = 2.4, | |
| | 7.6 Hz, 1H), 6.84 (d, J = 8.8 Hz, 1H), 5.10 (dd, J = 5.2, 13.1 Hz, 1H), | |
| | 4.46-4.35 (m, 3H), 4.25 (d, J = 17.2 Hz, 1H), 3.40 (d, J = 6.0 Hz, 3H), | |
| | 2.94-2.89 (m, 1H), 2.85 (s, 4H), 2.79-2.73 (m, 2H), 2.64-2.56 (m, | |
| | 4H), 2.46 (s, 3H), 2.43 (d, J = 4.4 Hz, 1H), 2.29 (d, J = 6.4 Hz, 2H), | |
| | 2.02-1.96 (m, 1H), 1.87 (d, J = 11.2 Hz, 2H), 1.78-1.68 (m, 1H), | |
| | 1.40-1.30 (m, 2H) | |
| 26 | ¹H NMR (400 MHz, DMSO-d₆) δ 10.99 (s, 1H), 10.03 (s, 1H), 8.85- | A |
| | 8.76 (m, 2H), 8.47-8.42 (m, 1H), 7.82 (s, 1H), 7.74 (d, J = 8.4 Hz, 1H), | |
| | 7.65 (d, J = 8.0 Hz, 1H), 7.59-7.54 (m, 1H), 7.52 (dd, J = 2.4, 10.0 Hz, | |
| | 1H), 7.28 (d, J = 8.4 Hz, 1H), 6.97 (dt, J = 2.8, 7.5 Hz, 1H), 6.79 (d, J = | |
| | 8.4 Hz, 1H), 5.14-5.06 (m, 1H), 4.45-4.36 (m, 3H), 4.26 (d, J = 17.2 | |
| | Hz, 1H), 3.42-3.38 (m, 2H), 3.09 (d, J = 1.6 Hz, 2H), 2.89 (d, J = 12.4 | |
| | Hz, 1H), 2.81-2.71 (m, 4H), 2.61 (s, 2H), 2.56 (d, J = 1.6 Hz, 2H), | |
| | 2.54 (s, 2H), 2.03-1.97 (m, 1H), 1.88 (d, J = 12.4 Hz, 2H), 1.81-1.63 | |
| | (m, 5H), 1.41-1.33 (m, 2H), 1.30 (d, J = 6.4 Hz, 6H) | |
| 27 | ¹H NMR (400 MHz, DMSO-d₆) δ 11.14-10.95 (m, 1H), 10.00 (s, 1H), | A |
| | 8.85-8.79 (m, 1H), 8.77-8.71 (m, 1H), 8.47-8.40 (m, 1H), 8.16 (s, | |
| | 2H), 7.82 (s, 1H), 7.71 (d, J = 8.4 Hz, 1H), 7.63 (d, J = 8.4 Hz, 1H), | |
| | 7.52 (dd, J = 2.4, 10.0 Hz, 1H), 7.05-6.82 (m, 3H), 6.64 (s, 1H), 5.06- | |
| | 4.92 (m, 1H), 4.38 (s, 2H), 4.09 ( d, J = 12.8 Hz, 2H), 3.92 (s, 3H), | |
| | 3.67-3.57 (m, 3H), 3.10-2.80 (m, 8H), 2.28-2.20 (m, 2H), 2.11-1.78 (m, | |
| | 7H), 1.77-1.62 (m, 4H), 1.52-1.32 (m, 6H), 1.24-1.10 (m, 2H) | |
| 28 | ¹H NMR (400 MHz, DMSO-d₆) δ 10.99 (s, 1H), 9.96 (s, 1H), 8.80 (s, | |
| | 1H), 8.53 (d, J = 8.4 Hz, 1H), 8.43 (t, J = 6.4 Hz, 1H), 8.28 (s, 1H), 7.81 | |
| | (s, 1H), 7.71 (d, J = 8.4 Hz, 1H), 7.64 (d, J = 8.0 Hz, 1H), 7.52 (d, J = | |
| | 8.8 Hz, 2H), 7.27 (d, J = 8.0 Hz, 1H), 7.01-6.93 (m, 1H), 6.70 (d, J = | |
| | 8.4 Hz, 1H), 5.10 (dd, J = 4.4, 12.8 Hz, 1H), 4.45-4.34 (m, 3H), 4.30- | |
| | 4.22 (m, 1H), 3.04-2.85 (m, 5H), 2.79-2.71 (m, 2H), 2.68-2.59 (m, | |
| | 2H), 2.35-2.22 (m, 4H), 2.08 (t, J = 10.8 Hz, 2H), 2.02-1.96 (m, 1H), | |
| | 1.86 (d, J = 10.4 Hz, 2H), 1.80-1.65 (m, 5H), 1.39-1.27 (m, 2H), 1.08 | |
| | (s, 2H), 0.99 (d, J = 5.6 Hz, 2H) | |
| 29 | ¹H NMR (400 MHz, DMSO-d₆) δ 10.99 (s, 1H), 10.35 (s, 1H), 8.93 (s, | A |
| | 1H), 8.64 (d, J = 8.4 Hz, 1H), 8.54-8.48 (m, 1H), 8.17 (s, 1H), 7.91 (d, | |
| | J = 8.8 Hz, 1H), 7.88-7.82 (m, 2H), 7.65 (d, J = 8.0 Hz, 1H), 7.53 (dd, | |
| | J = 2.4, 10.0 Hz, 1H), 7.33 (d, J = 8.8 Hz, 1H), 7.27 (d, J = 8.0 Hz, 1H), | |
| | 6.97 (dt, J = 2.4, 7.2 Hz, 1H), 5.10 (dd, J = 5.2, 13.2 Hz, 1H), 4.48- | |
| | 4.37 (m, 3H), 4.25 (d, J = 17.6 Hz, 1H), 3.45-3.38 (m, 3H), 3.03 (d, J = | |
| | 10.0 Hz, 2H), 2.95-2.80 (m, 2H), 2.75 (t, J = 11.2 Hz, 2H), 2.63-2.53 | |
| | (m, 2H), 2.47-2.42 (m, 1H), 2.27 (d, J = 6.8 Hz, 2H), 2.08-2.00 (m, | |
| | 2H), 1.87 (d, J = 11.6 Hz, 2H), 1.76 (d, J = 1.2 Hz, 4H), 1.33 (d, J = 9.2 | |
| | Hz, 2H) | |
| 30 | ¹H NMR (400 MHz, DMSO-d₆) δ 10.96 (s, 1H), 10.03 (s, 1H), 8.86- | A |
| | 8.74 (m, 2H), 8.44 (dd, J = 6.0, 7.2 Hz, 1H), 7.82 (s, 1H), 7.74 (dd, J = | |
| | 8.8, 10.8 Hz, 2H), 7.60-7.49 (m, 2H), 7.00-6.90 (m, 2H), 6.83 (d, J = | |
| | 8.4 Hz, 1H), 5.07 (dd, J = 4.8, 13.2 Hz, 1H), 4.51-4.43 (m, 2H), 4.39 | |
| | (s, 2H), 4.26 (d, J = 17.2 Hz, 1H), 4.14-4.05 (m, 1H), 3.01-2.90 (m, | |
| | 3H), 2.88-2.80 (m, 3H), 2.60 (d, J = 1.6 Hz, 1H), 2.59-2.52 (m, 5H), | |
| | 2.40-2.30 (m, 2H), 2.02-1.92 (m, 2H), 1.87-1.68 (m, 6H), 1.31 (t, | |
| | J = 7.6 Hz, 3H), 1.19-1.09 (m, 2H) | |
| 31 | ¹H NMR (400 MHz, DMSO-d₆) δ 10.98 (s, 1H), 10.01 (s, 1H), 8.92- | A |
| | 8.71 (m, 2H), 8.44 (dd, J = 6.0, 7.2 Hz, 1H), 8.16 (s, 1H), 7.82 (s, 1H), | |
| | 7.72 (d, J = 8.8 Hz, 1H), 7.57 (d, J = 8.4 Hz, 1H), 7.52 (dd, J = 2.4, 10.4 | |
| | Hz, 1H), 6.96 (dt, J = 2.4, 7.6 Hz, 1H), 6.89 (d, J = 7.6 Hz, 1H), 6.80 (d, | |
| | J = 8.4 Hz, 1H), 5.03 (dd, J = 5.2, 13.2 Hz, 1H), 4.44-4.35 (m, 3H), | |
| | 4.23 (d, J = 16.8 Hz, 1H), 3.54-3.47 (m, 4H), 3.04-2.96 (m, 2H), 2.95- | |
| | 2.88 (m, 1H), 2.86-2.72 (m, 5H), 2.60 (d, J = 2.4 Hz, 1H), 2.53 (s, | |
| | 3H), 2.40 (dd, J = 4.4, 12.8 Hz, 1H), 2.26 (d, J = 6.4 Hz, 2H), 2.12- | |
| | 2.03 (m, 2H), 1.99-1.92 (m, 1H), 1.83 (d, J = 10.4 Hz, 2H), 1.68 (s, | |
| | 5H), 1.30 (t, J = 7.6 Hz, 3H) | |
| 32 | ¹H NMR (400 MHz, DMSO-d₆) δ 10.99 (s, 1H), 9.99 (s, 1H), 8.80 (s, | A |
| | 1H), 8.73 (d, J = 8.8 Hz, 1H), 8.43 (dd, J = 6.0, 7.2 Hz, 1H), 8.18 (s, | |
| | 1H), 7.81 (s, 1H), 7.71 (d, J = 8.4 Hz, 1H), 7.64 (d, J = 8.0 Hz, 1H), | |
| | 7.56 (d, J = 8.4 Hz, 1H), 7.51 (dd, J = 2.4, 10.0 Hz, 1H), 7.25 (d, J = 8.0 | |
| | Hz, 1H), 6.96 (dt, J = 2.4, 7.6 Hz, 1H), 6.80 (d, J = 8.4 Hz, 1H), 5.10 | |
| | (dd, J = 5.2, 13.2 Hz, 1H), 4.44-4.36 (m, 3H), 4.25 (d, J = 17.6 Hz, | |
| | 1H), 3.01 (d, J = 10.4 Hz, 2H), 2.96-2.86 (m, 1H), 2.82-2.65 (m, 6H), | |
| | 2.64-2.55 (m, 2H), 2.48-2.39 (m, 1H), 2.33-2.27 (m, 2H), 2.10 (t, J = | |
| | 8.4 Hz, 2H), 2.03-1.96 (m, 1H), 1.85 (d, J = 11.6 Hz, 2H), 1.77 (td, | |
| | J = 7.2, 14.8 Hz, 3H), 1.71-1.63 (m, 4H), 1.38-1.26 (m, 2H), 0.99 | |
| | (t, J = 7.2 Hz, 3H) | |
| 33 | ¹H NMR (400 MHz, DMSO-d₆) δ 10.96 (s, 1H), 9.986 (s, 1H), 8.80 (s, | A |
| | 1H), 8.732 (d, J = 8.8 Hz, 1H), 8.48-8.40 (m, 1H), 7.80 (s, 1H), 7.72 | |
| | (d, J = 8.4 Hz, 1H), 7.56-7.48 (m, 2H), 7.36 (d, J = 8. 0 Hz, 1H), 6.96 | |
| | (dt, J = 2.8, 7.6 Hz, 1H), 6.80 (d, J = 8.4 Hz, 1H), 6.60 (t, J = 8.0 Hz, | |
| | 1H), 5.056 (dd, J = 5.2, 13.2 Hz, 1H), 4.478-4.40 (m, 1H), 4.40 (s, | |
| | 2H), 4.28 (d, J = 16.8 Hz, 1H), 4.16 (t, J = 6.8 Hz, 2H), 3.72 (d, J = 4.4 | |
| | Hz, 2H), 3.02-2.84 (m, 4H), 2.68-2.56 (m, 4H), 2.52 (s, 2H), 2.48- | |
| | 2.32 (m, 2H), 2.08 (t, J = 10.8 Hz, 2H), 2.00-1.92 (m, 1H), 1.72-1.56 | |
| | (m, 4H) | |
| 34 | ¹H NMR (400 MHz, DMSO-d₆) δ 11.11-11.04 (m, 1H), 9.98 (s, 1H), | A |
| | 8.81 (s, 1H), 8.73 (d, J = 8.4 Hz, 1H), 8.48-8.42 (m, 1H), 7.82 (s, 1H), | |
| | 7.72 (d, J = 8.4 Hz, 1H), 7.64 (d, J = 8.4 Hz, 1H), 7.58-7.50 (m, 2H), | |
| | 6.96 (dt, J = 2.8, 7.2 Hz, 1H), 6.85-6.77 (m, 2H), 6.65 (dd, J = 1.6, 8.4 | |
| | Hz, 1H), 5.06 (dd, J = 5.2, 12.8 Hz, 1H), 4.39 (s, 2H), 4.15 (t, J = 8.4 | |
| | Hz, 2H), 3.71 (dd, J = 5.2, 8.0 Hz, 2H), 3.03-2.94 (m, 3H), 2.69-2.59 | |
| | (m, 4H), 2.54 (s, 1H), 2.51 (s, 5H), 2.15-2.08 (m, 2H), 1.73-1.61 (m, | |
| | 4H) | |
| 35 | ¹H NMR (400 MHz, DMSO-d₆) δ 10.99 (s, 1H), 10.06 (s, 1H), 8.83 (s, | A |
| | 1H), 8.76 (d, J = 8.4 Hz, 1H), 8.49-8.42 (m, 1H), 8.19 (s, 1H), 7.83 (s, | |
| | 1H), 7.73 (d, J = 8.4 Hz, 1H), 7.69 (d, J = 8.4 Hz, 1H), 7.65 (d, J = 8.0 | |
| | Hz, 1H), 7.52 (dd, J = 2.4, 10.0 Hz, 1H), 7.27 (d, J = 8.0 Hz, 1H), 6.99- | |
| | 6.93 (m, 2H), 5.10 (dd, J = 4.8, 13.2 Hz, 1H), 4.56 (s, 2H), 4.45-4.36 | |
| | (m, 3H), 4.25 (d, J = 17.6 Hz, 1H), 3.35 (s, 3H), 3.00 (d, J = 10.0 Hz, | |
| | 2H), 2.96-2.85 (m, 2H), 2.81-2.71 (m, 3H), 2.64-2.55 (m, 2H), 2.49- | |
| | 2.37 (m, 2H), 2.26 (d, J = 6.4 Hz, 2H), 2.07-1.96 (m, 3H), 1.87 (d, | |
| | J = 11.2 Hz, 2H), 1.68 (s, 4H), 1.33 (d, J = 10.8 Hz, 2H) | |
| 36 | ¹H NMR (400 MHz, DMSO-d₆) δ 10.99 (s, 1H), 9.99 (s, 1H), 8.81 (s, | A |
| | 1H), 8.73 (d, J = 8.4 Hz, 1H), 8.44 (dd, J = 6.0, 7.2 Hz, 1H), 8.14 (s, | |
| | 1H), 7.82 (s, 1H), 7.72 (d, J = 8.4 Hz, 1H), 7.57-7.50 (m, 2H), 7.46- | |
| | 7.41 (m, 1H), 7.30 (d, J = 7.2 Hz, 1H), 7.16 (d, J = 7.6 Hz, 1H), 6.96 | |
| | (dt, J = 2.8, 7.2 Hz, 1H), 6.83 (d, J = 8.4 Hz, 1H), 5.12 (dd, J = 5.2, 13.2 | |
| | Hz, 1H), 4.46-4.28 (m, 4H), 3.43-3.39 (m, 2H), 3.03 (d, J = 9.6 Hz, | |
| | 2H), 2.95-2.88 (m, 1H), 2.82-2.69 (m, 3H), 2.64-2.54 (m, 2H), 2.51 | |
| | (s, 3H), 2.31 (d, J = 6.4 Hz, 2H), 2.20-2.09 (m, 2H), 2.03-1.97 (m, | |
| | 1H), 1.85 (d, J = 12.4 Hz, 2H), 1.77-1.63 (m, 5H), 1.36-1.26 (m, 2H) | |
| 37 | ¹H NMR (400 MHz, DMSO-d₆) δ 11.00 (s, 1H), 10.24 (s, 1H), 8.88 (s, | A |
| | 1H), 8.80 (d, J = 8.4 Hz, 1H), 8.52-8.40 (m, 1H), 7.92-7.80 (m, 2H), | |
| | 7.76-7.72 (m, 1H), 7.68-7.60 (m, 1H), 7.532 (dd, J = 2.8, 9.6 Hz, | |
| | 1H), 7.36-7.02 (m, 3H), 7.00-6.92 (m, 1H), 5.16-5.04 (m, 1H), 4.48- | |
| | 4.36 (m, 3H), 4.28 (d, J = 17.6 Hz, 1H), 3.06-2.88 (m, 4H), 2.80- | |
| | 2.72 (m, 2H), 2.68-2.60 (m, 1H), 2.32-2.20 (m, 2H), 2.10-1.96 (m, | |
| | 4H), 1.92-1.84 (m, 3H), 1.80-1.56 (m, 5H), 1.28-1.20 (m, 3H) | |
| 38 | ¹H NMR (400 MHz, DMSO-d₆) δ 10.94 (s, 1H), 9.98 (s, 1H), 8.81 (s, | A |
| | 1H), 8.73 (d, J = 8.4 Hz, 1H), 8.44 (dd, J = 6.0, 7.2 Hz, 1H), 8.22 (s, | |
| | 1H), 7.82 (s, 1H), 7.71 (d, J = 8.8 Hz, 1H), 7.58-7.46 (m, 3H), 6.96 (dt, | |
| | J = 2.8, 7.2 Hz, 1H), 6.82 (d, J = 8.4 Hz, 1H), 6.55-6.43 (m, 2H), 5.03 | |
| | (dd, J = 5.2, 13.2 Hz, 1H), 4.38 (s, 2H), 4.34-4.27 (m, 1H), 4.21-4.14 | |
| | (m, 1H), 4.04 (t, J = 7.6 Hz, 2H), 3.62-3.54 (m, 4H), 3.05-2.84 (m, | |
| | 5H), 2.69-2.59 (m, 4H), 2.35 (dd, J = 4.4, 13.2 Hz, 1H), 2.15-2.07 | |
| | (m, 2H), 1.99-1.91 (m, 1H), 1.73-1.58 (m, 4H) | |
| 39 | ¹H NMR (400 MHz, DMSO-d₆) δ 11.08 (s, 1H), 9.96 (s, 1H), 8.80 (s, | A |
| | 1H), 8.72 (d, J = 8.8 Hz, 1H), 8.48-8.40 (m, 1H), 8.20 (s, 1H), 7.80 (s, | |
| | 1H), 7.72 (d, J = 8.4 Hz, 1H), 7.64 (d, J = 8.4 Hz, 1H), 7.56-7.48 (m, | |
| | 2H), 6.96 (dt, J = 2.8, 7.6 Hz, 1H), 6.80 (d, J = 8.4 Hz, 1H), 6.76 (d, J = | |
| | 1.6 Hz, 1H), 6.64 (dd, J = 2.0, 8.4 Hz, 1H), 5.06 (dd, J = 5.6, 12.8 Hz, | |
| | 1H), 4.36 (s, 2H), 4.16 (t, J = 8.0 Hz, 2H), 3.72 (dd, J = 6.0, 8.0 Hz, | |
| | 3H), 3.02 (d, J = 10.8 Hz, 2H), 2.96-2.76 (m, 3H), 2.72-2.56 (m, 2H), | |
| | 2.52-2.48 (m, 2H), 2.40-2.32 (m, 2H), 2.12-1.96 (m, 3H), 1.88- | |
| | 1.76 (m, 2H), 1.72-1.56 (m, 4H) | |
| 40 | ¹H NMR (400 MHz, DMSO-d₆) δ 10.97 (s, 1H), 9.98 (s, 1H), 8.82 (s, | A |
| | 1H), 8.73 (d, J = 8.8 Hz, 1H), 8.45 (dd, J = 6.0, 7.2 Hz, 1H), 7.82 (s, | |
| | 1H), 7.72 (d, J = 8.4 Hz, 1H), 7.57-7.48 (m, 3H), 6.96 (dt, J = 2.8, 7.6 | |
| | Hz, 1H), 6.82 (d, J = 8.4 Hz, 1H), 6.64 (d, J = 8.4 Hz, 1H), 5.06 (dd, J = | |
| | 5.2, 13.2 Hz, 1H), 4.39 (s, 2H), 4.36-4.25 (m, 3H), 4.17 (d, J = 17.2 | |
| | Hz, 1H), 3.84 (t, J = 5.6 Hz, 2H), 2.99 (d, J = 10.8 Hz, 2H), 2.95-2.85 | |
| | (m, 2H), 2.68-2.65 (m, 2H), 2.62-2.53 (m, 2H), 2.51 (s, 3H), 2.45- | |
| | 2.35 (m, 1H), 2.14 (t, J = 10.8 Hz, 2H), 1.97 (d, J = 5.6 Hz, 1H), 1.74- | |
| | 1.58 (m, 4H) | |
| 41 | ¹H NMR (400 MHz, DMSO-d₆) δ 10.98 (s, 1H), 10.02 (s, 1H), 8.84 (s, | A |
| | 1H), 8.61 (d, J = 8.4 Hz, 1H), 8.43 (dd, J = 6.0, 7.2 Hz, 1H), 7.82 (s, | |
| | 1H), 7.73 (d, J = 8.4 Hz, 1H), 7.57-7.49 (m, 2H), 7.41 (d, J = 8.4 Hz, | |
| | 1H), 7.25 (dd, J = 2.0, 8.4 Hz, 1H), 7.16 (d, J = 2.0 Hz, 1H), 6.98 (dt, J = | |
| | 2.4, 7.6 Hz, 1H), 6.54 (d, J = 8.0 Hz, 1H), 5.10 (dd, J = 5.2, 13.2 Hz, | |
| | 1H), 4.39 (s, 2H), 4.36-4.29 (m, 1H), 4.23-4.16 (m, 1H), 3.99 (s, 3H), | |
| | 3.76 (d, J = 12.0 Hz, 2H), 3.00-2.89 (m, 3H), 2.75-2.66 (m, 3H), 2.62- | |
| | 2.56 (m, 1H), 2.41-2.31 (m, 1H), 2.20 (d, J = 6.8 Hz, 2H), 2.04-1.93 | |
| | (m, 3H), 1.82 (d, J = 11.2 Hz, 2H), 1.76-1.56 (m, 5H), 1.29-1.16 (m, | |
| | 2H) | |
| 42 | ¹H NMR (400 MHz, DMSO-d₆) δ 10.98 (s, 1H), 10.16 (s, 1H), 8.86 (s, | A |
| | 1H), 8.72 (d, J = 8.4 Hz, 1H), 8.48 (t, J = 6.8 Hz, 1H), 7.84 (s, 1H), 7.79 | |
| | (d, J = 8.4 Hz, 1H), 7.69 (d, J = 8.8 Hz, 1H), 7.65 (d, J = 8.4 Hz, 1H), | |
| | 7.52 (dd, J = 2.0, 10.0 Hz, 1H), 7.28 (d, J = 8.0 Hz, 1H), 7.05 (d, J = 8.8 | |
| | Hz, 1H), 6.99-6.92 (m, 1H), 5.10 (dd, J = 4.4, 13.2 Hz, 1H), 4.50 (s, | |
| | 1H), 4.45-4.37 (m, 3H), 4.26 (d, J = 17.6 Hz, 1H), 3.46-3.37 (m, 3H), | |
| | 3.15-2.98 (m, 3H), 2.89 (d, J = 13.6 Hz, 1H), 2.76 (t, J = 11.6 Hz, 2H), | |
| | 2.63-2.55 (m, 2H), 2.45-2.36 (m, 3H), 2.26-2.15 (m, 1H), 1.99 (dd, J = | |
| | 4.8, 10.0 Hz, 1H), 1.91-1.84 (m, 2H), 1.81-1.73 (m, 4H), 1.41- | |
| | 1.29 (m, 2H) | |
| 43 | ¹H NMR (400 MHz, DMSO-d₆) δ 10.97 (s, 1H), 10.02 (s, 1H), 8.84- | A |
| | 8.74 (m, 2H), 8.47-8.41 (m, 1H), 7.82 (s, 1H), 7.72 (d, J = 8.4 Hz, 1H), | |
| | 7.59-7.48 (m, 2H), 7.42 (d, J = 8.4 Hz, 1H), 7.26 (d, J = 8.4 Hz, 1H), | |
| | 7.17 (d, J = 1.6 Hz, 1H), 6.97 (dt, J = 2.8, 7.6 Hz, 1H), 6.82 (d, J = 8.4 | |
| | Hz, 1H), 5.10 (dd, J = 4.8, 13.2 Hz, 1H), 4.41-4.30 (m, 3H), 4.24- | |
| | 4.17 (m, 1H), 3.77 (d, J = 11.6 Hz, 2H), 3.15 (d, J = 8.8 Hz, 3H), 2.97- | |
| | 2.90 (m, 1H), 2.88-2.68 (m, 6H), 2.59 (d, J = 17.2 Hz, 1H), 2.45 (s, | |
| | 1H), 2.41-2.29 (m, 3H), 2.02-1.96 (m, 1H), 1.87-1.69 (m, 7H), 1.31 | |
| | (t, J = 7.2 Hz, 3H), 1.27-1.22 (m, 1H) | |
| 44 | ¹H NMR (400 MHz, DMSO-d₆) δ 10.99 (s, 1H), 10.03 (s, 1H), 8.82 (s, | A |
| | 1H), 8.74 (d, J = 8.4 Hz, 1H), 8.44 (t, J = 6.8 Hz, 1H), 8.15 (s, 1H), 7.83 | |
| | (s, 1H), 7.72 (d, J = 8.4 Hz, 1H), 7.65 (dd, J = 3.6, 8.4 Hz, 2H), 7.53 | |
| | (dd, J = 2.4, 10.0 Hz, 1H), 7.31-7.22 (m, 1H), 7.02-6.87 (m, 2H), | |
| | 5.22-5.03 (m, 2H), 4.69-4.57 (m, 2H), 4.46-4.34 (m, 3H), 4.29- | |
| | 4.21 (m, 1H), 3.45-3.39 (m, 3H), 3.01 ( d, J = 10.4 Hz, 2H), 2.94- | |
| | 2.82 (m, 2H), 2.80-2.69 (m, 2H), 2.61 ( s, 1H), 2.57-2.53 (m, 1H), | |
| | 2.34-2.26 (m, 2H), 2.11-1.95 (m, 3H), 1.88 ( d, J = 12.4 Hz, 2H), | |
| | 1.75-1.66 (m, 4H), 1.41-1.26 (m, 2H) | |
| 45 | ¹H NMR (400 MHz, DMSO-d₆) δ 11.08 (s, 1H), 9.97 (s, 1H), 8.80 (s, | A |
| | 1H), 8.72 (d, J = 8.4 Hz, 1H), 8.44 (dd, J = 6.4, 7.2 Hz, 1H), 8.16 (s, | |
| | 1H), 7.81 (s, 1H), 7.71 (d, J = 8.8 Hz, 1H), 7.58-7.48 (m, 2H), 7.00- | |
| | 6.93 (m, 2H), 6.88 (dd, J = 8.0, 13.2 Hz, 2H), 6.81 (d, J = 8.4 Hz, 1H), | |
| | 5.35 (dd, J = 5.2, 12.4 Hz, 1H), 4.38 (s, 2H), 3.63 (s, 3H), 3.12 (d, J = | |
| | 8.4 Hz, 2H), 3.01 (d, J = 9.6 Hz, 2H), 2.90-2.83 (m, 1H), 2.75-2.61 | |
| | (m, 6H), 2.59 (s, 1H), 2.54 (s, 1H), 2.28 (d, J = 5.2 Hz, 2H), 2.12-2.03 | |
| | (m, 2H), 2.02-1.96 (m, 1H), 1.85 (d, J = 11.6 Hz, 2H), 1.73-1.60 (m, | |
| | 5H), 1.42-1.26 (m, 2H) | |
| 46 | ¹H NMR (400 MHz, DMSO-d₆) δ 10.98 (s, 1H), 10.04 (s, 1H), 8.83 (s, | A |
| | 1H), 8.60 (d, J = 8.5 Hz, 1H), 8.43 (t, J = 6.4 Hz, 1H), 7.82 (s, 1H), 7.73 | |
| | (d, J = 8.4 Hz, 1H), 7.65 (d, J = 8.0 Hz, 1H), 7.51 (d, J = 8.0 Hz, 2H), | |
| | 7.27 (d, J = 8.4 Hz, 1H), 6.98 (dt, J = 2.4, 7.6 Hz, 1H), 6.57 (d, J = 8.0 | |
| | Hz, 1H), 5.10 (dd, J = 5.2, 13.2 Hz, 1H), 4.45-4.36 (m, 3H), 4.31- | |
| | 4.22 (m, 1H), 4.00 (s, 3H), 3.41 (d, J = 6.4 Hz, 2H), 3.26-3.16 (m, 2H), | |
| | 2.97-2.83 (m, 2H), 2.77 (t, J = 11.6 Hz, 2H), 2.65-2.52 (m, 4H), 2.48- | |
| | 2.39 (m, 2H), 2.03-1.97 (m, 1H), 1.91-1.72 (m, 7H), 1.38 (d, J = | |
| | 10.8 Hz, 2H) | |
| 47 | ¹H NMR (400 MHz, DMSO-d₆) δ 10.95 (s, 1H), 9.94 (s, 1H), 8.79 (s, | A |
| | 1H), 8.66 (d, J = 8.8 Hz, 1H), 8.44 (dd, J = 6.0, 7.2 Hz, 1H), 8.14 (s, | |
| | 1H), 7.81 (s, 1H), 7.72 (dd, J = 8.8, 14.0 Hz, 2H), 7.52 (dd, J = 2.4, 10.0 | |
| | Hz, 1H), 7.46 (d, J = 8.4 Hz, 1H), 6.96 (dt, J = 2.4, 7.6 Hz, 1H), 6.90 (d, | |
| | J = 9.2 Hz, 1H), 6.84 (d, J = 8.4 Hz, 1H), 5.07 (dd, J = 5.2, 13.2 Hz, | |
| | 1H), 4.44 ( d, J = 12.8 Hz, 2H), 4.38 (s, 2H), 4.26 (d, J = 17.2 Hz, 1H), | |
| | 4.09 (d, J = 17.2 Hz, 1H), 3.02-2.89 (m, 3H), 2.88-2.80 (m, 4H), 2.63- | |
| | 2.51 (m, 5H), 2.46 (s, 3H), 2.40-2.29 (m, 1H), 2.23 ( d, J = 6.8 Hz, | |
| | 2H), 2.01-1.86 (m, 2H), 1.82 ( d, J = 13.2 Hz, 2H), 1.20-1.00 (m, 2H) | |
| 48 | ¹H NMR (400 MHz, DMSO-d₆) δ 10.94 (s, 1H), 9.93 (s, 1H), 8.79 (s, | A |
| | 1H), 8.65 (d, J = 8.4 Hz, 1H), 8.44 (dd, J = 6.0, 7.2 Hz, 1H), 7.81 (s, | |
| | 1H), 7.70 (d, J = 8.4 Hz, 1H), 7.54-7.43 (m, 3H), 7.07-7.01 (m, 2H), | |
| | 6.96 (dt, J = 2.8, 7.6 Hz, 1H), 6.84 (d, J = 8.8 Hz, 1H), 5.04 (dd, J = 5.2, | |
| | 13.2 Hz, 1H), 4.38 (s, 2H), 4.35-4.27 (m, 1H), 4.23-4.15 (m, 1H), | |
| | 3.88 (d, J = 12.4 Hz, 2H), 2.91-2.78 (m, 7H), 2.64-2.52 (m, 4H), 2.45 | |
| | (s, 3H), 2.43-2.28 (m, 2H), 2.23 (d, J = 6.8 Hz, 2H), 2.00-1.90 (m, | |
| | 1H), 1.81 (d, J = 10.4 Hz, 3H), 1.30-1.10 (m, 2H) | |
| 49 | ¹H NMR (400 MHz, DMSO-d₆) δ 10.96 (s, 1H), 9.96 (s, 1H), 8.76 (s, | A |
| | 1H), 8.64 (d, J = 8.4 Hz, 1H), 8.44 (dd, J = 6.0, 7.2 Hz, 1H), 8.16 (s, | |
| | 1H), 7.80 (s, 1H), 7.72 (d, J = 8.8 Hz, 1H), 7.52 (dd, J = 2.8, 10.0 Hz, | |
| | 1H), 7.48 (d, J = 8.8 Hz, 1H), 7.40 (d, J = 8.0 Hz, 1H), 6.96 (dt, J = 2.8, | |
| | 7.6 Hz, 1H), 6.84 (d, J = 8.4 Hz, 1H), 6.60 (t, J = 8.0 Hz, 1H), 5.02 (dd, | |
| | J = 5.2, 13.2 Hz, 1H), 4.44 (d, J = 16.8 Hz, 1H), 4.36 (s, 2H), 4.28 (d, J = | |
| | 16.8 Hz, 1H), 4.20-4.12 (m, 2H), 3.76 (d, J = 4.4 Hz, 2H), 3.04-2.88 | |
| | (m, 2H), 2.88-2.80 (m, 4H), 2.72-2.68 (m, 2H), 2.64-2.56 (m, 4H), | |
| | 2.52 (s, 2H), 2.48 (s, 3H), 2.00-1.92 (m, 1H) | |
| 50 | ¹H NMR (400 MHz, DMSO-d₆) δ 10.96 (s, 1H), 9.96 (s, 1H), 8.76 (s, | A |
| | 1H), 8.64 (d, J = 8.4 Hz, 1H), 8.44 (dd, J = 6.0, 7.2 Hz, 1H), 8.12 (s, | |
| | 1H), 7.80 (s, 1H), 7.72 (d, J = 8.8 Hz, 1H), 7.52 (dd, J = 2.8, 10.0 Hz, | |
| | 1H), 7.48 (d, J = 8.8 Hz, 1H), 6.96 (dt, J = 2.8, 7.6 Hz, 1H), 6.88 (d, J = | |
| | 7.6 Hz, 1H), 6.84 (d, J = 8.4 Hz, 1H), 5.00 (dd, J = 5.2, 13.2 Hz, 1H), | |
| | 4.48-4.32 (m, 3H), 4.28-4.20 (m, 1H), 3.52 (d, J = 8.8 Hz, 3H), 2.96- | |
| | 2.88 (m, 1H), 2.84 (s, 4H), 2.76 (t, J = 11.6 Hz, 2H), 2.60-2.56 (m, | |
| | 4H), 2.52 (s, 3H), 2.48 (s, 3H), 2.40 (dd, J = 4.4, 13.2 Hz, 1H), 2.36- | |
| | 2.24 (m, 2H), 2.00-1.92 (m, 1H), 1.88-1.80 (m, 2H), 1.76-1.68 (m, | |
| | 1H), 1.36-1.20 (m, 2H) | |
| 51 | ¹H NMR (400 MHz, DMSO-d₆) δ 10.96 (s, 1H), 9.96 (s, 1H), 8.76 (s, | A |
| | 1H), 8.64 (d, J = 8.4 Hz, 1H), 8.48-8.40 (m, 1H), 8.12 (s, 1H), 7.80 (s, | |
| | 1H), 7.72 (d, J = 8.4 Hz, 1H), 7.52 (dd, J = 2.4, 10.0 Hz, 1H), 7.48- | |
| | 7.40 (m, 2H), 7.30 (d, J = 7.6 Hz, 1H), 7.16 (d, J = 8.0 Hz, 1H), 6.96 | |
| | (dt, J = 2.4, 7.6 Hz, 1H), 6.88-6.80 (m, 1H), 5.12 (dd, J = 5.2, 13.2 Hz, | |
| | 1H), 4.56-4.24 (m, 4H), 3.44-3.36 (m, 4H), 2.96-2.92 (m, 1H), 2.88 | |
| | (s, 4H), 2.76 (q, J = 10.8 Hz, 2H), 2.68-2.56 (m, 4H), 2.48 (s, 3H), | |
| | 2.40-2.28 (m, 2H), 2.04-1.96 (m, 1H), 1.84 (d, J = 12.4 Hz, 2H), | |
| | 1.80-1.68 (m, 1H), 1.36-1.20 (m, 2H) | |
| 52 | ¹H NMR (400 MHz, DMSO-d₆) δ 11.06 (s, 1H), 9.98 (s, 1H), 8.82 (s, | A |
| | 1H), 8.73 (d, J = 8.4 Hz, 1H), 8.45 (dd, J = 6.0, 7.6 Hz, 1H), 8.16 (s, | |
| | 1H), 7.82 (s, 1H), 7.72 (d, J = 8.4 Hz, 1H), 7.58-7.50 (m, 2H), 7.00- | |
| | 6.87 (m, 3H), 6.82 (d, J = 8.4 Hz, 1H), 6.63 (dd, J = 2.0, 8.4 Hz, 1H), | |
| | 5.26 (dd, J = 5.2, 12.8 Hz, 1H), 4.59 (td, J = 7.2, 14.0 Hz, 1H), 4.39 (s, | |
| | 2H), 3.56 ( d, J = 11.6 Hz, 2H), 2.99 ( d, J = 11.2 Hz, 2H), 2.92-2.83 | |
| | (m, 1H), 2.69-2.56 (m, 5H), 2.51 ( s, 3H), 2.24 ( d, J = 6.8 Hz, 2H), | |
| | 2.09-1.95 (m, 3H), 1.86-1.79 (m, 2H), 1.74-1.59 (m, 5H), 1.45 (d, | |
| | J = 6.8 Hz, 6H), 1.33-1.22 (m, 2H) | |
| 53 | ¹H NMR (400 MHz, DMSO-d₆) δ 11.00 (s, 1H), 10.02 (s, 1H), 8.82 (s, | A |
| | 1H), 8.77 (d, J = 8.8 Hz, 1H), 8.47-8.42 (m, 1H), 8.14 (d, J = 0.8 Hz, | |
| | 1H), 7.82 (s, 1H), 7.73 (d, J = 8.4 Hz, 1H), 7.57-7.50 (m, 2H), 7.46- | |
| | 7.41 (m, 1H), 7.30 (d, J = 7.2 Hz, 1H), 7.17 (d, J = 8.0 Hz, 1H), 7.00- | |
| | 6.94 (m, 1H), 6.83 (d, J = 8.4 Hz, 1H), 5.12 (dd, J = 5.2, 13.2 Hz, 1H), | |
| | 4.47-4.26 (m, 4H), 3.45-3.36 (m, 4H), 3.14 (d, J = 9.2 Hz, 2H), 2.93 | |
| | (s, 1H), 2.86-2.70 (m, 5H), 2.64-2.53 (m, 2H), 2.47 (d, J = 4.4 Hz, | |
| | 2H), 2.39-2.33 (m, 1H), 2.00 (s, 1H), 1.86 (d, J = 12.0 Hz, 2H), 1.80- | |
| | 1.71 (m, 4H), 1.37-1.26 (m, 5H) | |
| 54 | ¹H NMR (400 MHz, DMSO-d₆) δ 10.97 (s, 1H), 10.02 (s, 1H), 8.81 (s, | A |
| | 1H), 8.73 (d, J = 8.4 Hz, 1H), 8.44 (dd, J = 6.0, 7.2 Hz, 1H), 7.82 (s, | |
| | 1H), 7.72 (d, J = 8.8 Hz, 1H), 7.52 (dd, J = 2.4, 10.0 Hz, 2H), 7.03 (s, | |
| | 1H), 6.97 (dt, J = 2.4, 7.2 Hz, 1H), 6.87 (d, J = 8.4 Hz, 1H), 5.06 (dd, J = | |
| | 5.2, 13.2 Hz, 1H), 4.39 (s, 2H), 4.34 (d, J = 17.6 Hz, 1H), 4.22-4.16 | |
| | (m, 1H), 3.42 (d, J = 6.0 Hz, 3H), 2.95-2.85 (m, 2H), 2.84-2.64 (m, | |
| | 4H), 2.63-2.56 (m, 5H), 2.54 (s, 3H), 2.47-2.35 (m, 2H), 2.03-1.72 | |
| | (m, 9H), 1.49-1.35 (m, 2H) | |
| 55 | ¹H NMR (400 MHz, DMSO-d₆) δ 10.98 (s, 1H), 9.93 (s, 1H), 8.79 (s, | A |
| | 1H), 8.65 (d, J = 8.4 Hz, 1H), 8.44 (dd, J = 6.0, 7.2 Hz, 1H), 8.22 (s, | |
| | 1H), 7.81 (s, 1H), 7.70 (d, J = 8.8 Hz, 1H), 7.65 (d, J = 8.0 Hz, 1H), | |
| | 7.51 (dd, J = 2.4, 10.0 Hz, 1H), 7.45 (d, J = 8.4 Hz, 1H), 7.27 (d, J = 8.4 | |
| | Hz, 1H), 6.96 (dt, J = 2.4, 7.6 Hz, 1H), 6.84 (d, J = 8.4 Hz, 1H), 5.10 | |
| | (dd, J = 4.8, 13.2 Hz, 1H), 4.45-4.36 (m, 3H), 4.25 (d, J = 17.2 Hz, | |
| | 1H), 2.97-2.86 (m, 4H), 2.81-2.71 (m, 3H), 2.71-2.63 (m, 1H), 2.63- | |
| | 2.52 (m, 5H), 2.47 (s, 3H), 2.44-2.32 (m, 2H), 2.15-2.08 (m, 1H), | |
| | 2.03-1.92 (m, 2H), 1.80 (br d, J = 11.2 Hz, 1H), 1.69 (dd, J = 2.0, 3.6 | |
| | Hz, 1H), 1.39-1.25 (m, 2H), 1.08 (br d, J = 5.6 Hz, 3H) | |
| 56 | ¹H NMR (400 MHz, DMSO-d₆) δ 10.88 (d, J = 12.4 Hz, 1H), 9.98 (s, | A |
| | 1H), 8.80 (s, 1H), 8.73 (d, J = 8.4 Hz, 1H), 8.47-8.41 (m, 1H), 7.82 (s, | |
| | 1H), 7.74-7.66 (m, 2H), 7.58-7.49 (m, 2H), 6.96 (d, J = 2.4 Hz, 1H), | |
| | 6.87 (dd, J = 3.2, 8.8 Hz, 1H), 6.82 (d, J = 8.4 Hz, 1H), 4.69 (dd, J = | |
| | 5.2, 12.0 Hz, 1H), 4.52-4.40 (m, 3H), 4.38 (s, 2H), 3.02-2.91 (m, | |
| | 4H), 2.89-2.70 (m, 2H), 2.69-2.52 (m, 5H), 2.20 (d, J = 6.4 Hz, 2H), | |
| | 2.08-1.94 (m, 3H), 1.82 (d, J = 14.8 Hz, 3H), 1.73-1.58 (m, 4H), 1.39 | |
| | (dd, J = 6.8, 14.4 Hz, 3H), 1.16-1.04 (m, 2H) | |
| 57 | ¹H NMR (400 MHz, DMSO-d₆) δ 10.98 (s, 1H), 9.93 (s, 1H), 8.78 (s, | A |
| | 1H), 8.65 (d, J = 8.8 Hz, 1H), 8.44 (dd, J = 6.4, 7.2 Hz, 1H), 7.84-7.79 | |
| | (m, 1H), 7.70 (d, J = 8.4 Hz, 1H), 7.51 (dd, J = 2.4, 10.0 Hz, 1H), 7.47- | |
| | 7.39 (m, 2H), 7.30 (d, J = 7.2 Hz, 1H), 7.16 (d, J = 7.6 Hz, 1H), 6.99- | |
| | 6.92 (m, 1H), 6.84 (d, J = 8.8 Hz, 1H), 5.12 (dd, J = 4.8, 13.2 Hz, 1H), | |
| | 4.47-4.26 (m, 4H), 3.43-3.38 (m, 2H), 2.96-2.86 (m, 4H), 2.81- | |
| | 2.66 (m, 4H), 2.62-2.53 (m, 4H), 2.47 (s, 3H), 2.39-2.33 (m, 1H), | |
| | 2.13-2.06 (m, 1H), 2.03-1.90 (m, 2H), 1.79 (d, J = 14.0 Hz, 1H), 1.67 | |
| | (s, 1H), 1.36-1.21 (m, 2H), 1.07 (d, J = 5.6 Hz, 3H) | |
| 58 | ¹H NMR (400 MHz, DMSO-d₆) δ 10.80 (s, 1H), 10.00 (s, 1H), 8.84 (s, | A |
| | 1H), 8.72 (d, J = 8.8 Hz, 1H), 8.48-8.40 (m, 1H), 7.96-7.92 (m, 1H), | |
| | 7.834(s, 1H), 7.72 (d, J = 8.8 Hz, 1H), 7.52 (dd, J = 2.8, 10.0 Hz, 2H), | |
| | 7.40-7.36 (m, 1H), 6.96 (dt, J = 2.8, 7.6 Hz, 1H), 6.92-6.76 (m, 2H), | |
| | 4.40 (s, 2H), 4.32-4.24 (m, 2H), 3.72 (dd, J = 4.8, 11.6 Hz, 1H), 3.32- | |
| | 2.96 (m, 4H), 2.92-2.76 (m, 3H), 2.72-2.64 (m, 2H), 2.52 (d, J = 6.6 | |
| | Hz, 4H), 2.24-2.16 (m, 2H), 1.98 (dt, J = 4.4, 8.8 Hz, 2H), 1.88-1.68 | |
| | (m, 6H), 1.24-1.08 (m, 2H) | |
| 59 | ¹H NMR (400 MHz, DMSO-d₆) δ 10.99 (s, 1H), 10.22 (s, 1H), 8.89 (s, | A |
| | 1H), 8.59 (d, J = 8.8 Hz, 1H), 8.49 (t, J = 6.4 Hz, 1H), 8.43 (s, 1H), 7.87- | |
| | 7.78 (m, 2H), 7.71 (d, J = 9.2 Hz, 1H), 7.65 (d, J = 8.0 Hz, 1H), 7.56- | |
| | 7.49 (m, 1H), 7.35 (d, J = 9.2 Hz, 1H), 7.27 (d, J = 8.0 Hz, 1H), 7.00- | |
| | 6.93 (m, 1H), 5.10 (dd, J = 5.2, 13.2 Hz, 1H), 4.46-4.38 (m, 3H), 4.25 | |
| | (d, J = 17.2 Hz, 1H), 3.15 (s, 4H), 2.76 (t, J = 11.6 Hz, 3H), 2.58 (s, | |
| | 6H), 2.30 (d, J = 6.4 Hz, 2H), 2.05-1.69 (m, 5H), 1.34 (d, J = 11.2 Hz, | |
| | 2H), 1.23 (s, 1H) | |
| 60 | ¹H NMR (400 MHz, DMSO-d₆) δ 10.97 (s, 1H), 10.40 (s, 1H), 8.94 (s, | A |
| | 1H), 8.64 ( d, J = 8.4 Hz, 1H), 8.50 (s, 1H), 7.91-7.71 (m, 3H), 7.54 (d, | |
| | J = 10.0 Hz, 1H), 7.40 (s, 1H), 7.01-6.95 (m, 1H), 6.91 (d, J = 7.2 Hz, | |
| | 1H), 5.03 (d, J = 18.0 Hz, 1H), 4.50-4.36 (m, 3H), 4.26 (s, 1H), 3.53 | |
| | (s, 4H), 2.96-2.77 (m, 4H), 2.69-2.59 (m, 2H), 2.55 (s, 3H), 2.45- | |
| | 2.32 (m, 3H), 2.02-1.74 (m, 7H), 1.39 (s, 2H), 1.23 (s, 1H), 0.89-0.73 | |
| | (m, 1H) | |
| 61 | ¹H NMR (400 MHz, DMSO-d₆) δ 10.97 (s, 1H), 10.21 (s, 1H), 8.89 (s, | A |
| | 1H), 8.58 (d, J = 8.4 Hz, 1H), 8.52-8.46 (m, 1H), 7.88-7.78 (m, 2H), | |
| | 7.71 (d, J = 9.2 Hz, 1H), 7.52 (dd, J = 2.4, 10.0 Hz, 1H), 7.34 (d, J = 9.2 | |
| | Hz, 1H), 6.96 (dt, J = 2.8, 7.6 Hz, 1H), 6.89 (d, J = 7.6 Hz, 1H), 5.02 | |
| | (dd, J = 4.8, 13.2 Hz, 1H), 4.46-4.35 (m, 3H), 4.23 (d, J = 17.2 Hz, | |
| | 1H), 3.50 (d, J = 8.0 Hz, 2H), 3.14 (s, 4H), 2.96-2.85 (m, 1H), 2.77 (t, | |
| | J = 11.2 Hz, 2H), 2.63-2.52 (m, 8H), 2.43-2.38 (m, 1H), 2.27 (d, J = | |
| | 6.8 Hz, 2H), 2.00-1.92 (m, 1H), 1.88-1.69 (m, 3H), 1.35-1.21 (m, | |
| | 2H) | |
| 62 | ¹H NMR (400 MHz, DMSO-d₆) δ 10.98 (s, 1H), 9.95 (s, 1H), 8.78 (s, | |
| | 1H), 8.65 (d, J = 8.8 Hz, 1H), 8.44 (t, J = 6.8 Hz, 1H), 7.81 (s, 1H), 7.71 | |
| | (d, J = 8.8 Hz, 1H), 7.65 (d, J = 8.0 Hz, 1H), 7.52 (dd, J = 2.4, 10.0 Hz, | |
| | 1H), 7.46 (d, J = 8.8 Hz, 1H), 7.28 (d, J = 8.4 Hz, 1H), 6.96 (dt, J = 2.8, | |
| | 7.6 Hz, 1H), 6.85 (d, J = 8.8 Hz, 1H), 5.10 (dd, J = 4.8, 13.2 Hz, 1H), | |
| | 4.45-4.35 (m, 3H), 4.30-4.22 (m, 1H), 3.43 (s, 2H), 2.99-2.69 (m, | |
| | 7H), 2.64-2.53 (m, 3H), 2.48 (s, 3H), 2.45-2.28 (m, 2H), 2.21-2.05 | |
| | (m, 1H), 2.03-1.92 (m, 2H), 1.81 (d, J = 11.6 Hz, 2H), 1.50-1.01 (m, | |
| | 6H) | |
| 63 | ¹H NMR (400 MHz, DMSO-d₆) δ 11.00 (s, 1H), 9.99 (s, 1H), 8.81 (s, | A |
| | 1H), 8.74 (d, J = 8.4 Hz, 1H), 8.44 (dd, J = 6.0, 7.2 Hz, 1H), 7.82 (s, | |
| | 1H), 7.72 (d, J = 8.4 Hz, 1H), 7.65 (d, J = 8.0 Hz, 1H), 7.55-7.49 (m, | |
| | 2H), 7.28 (d, J = 8.4 Hz, 1H), 7.01-6.93 (m, 1H), 6.83 (d, J = 8.8 Hz, | |
| | 1H), 5.10 (dd, J = 4.8, 13.4 Hz, 1H), 4.47-4.34 (m, 3H), 4.25 (d, J = | |
| | 17.2 Hz, 1H), 3.45-3.38 (m, 2H), 2.93-2.71 (m, 9H), 2.63-2.51 (m, | |
| | 6H), 2.48-2.40 (m, 2H), 2.04-1.94 (m, 1H), 1.92-1.70 (m, 3H), 1.32 | |
| | (t, J = 7.6 Hz, 5H) | |
| 64 | ¹H NMR (400 MHz, DMSO-d₆) δ 11.00 (s, 1H), 10.00 (s, 1H), 8.82 (s, | A |
| | 1H), 8.71 (d, J = 8.8 Hz, 1H), 8.49-8.40 (m, 1H), 7.83 (s, 1H), 7.72 (d, | |
| | J = 8.8 Hz, 1H), 7.65 (d, J = 8.0 Hz, 1H), 7.61-7.49 (m, 2H), 7.27 (d, J = | |
| | 8.4 Hz, 1H), 6.97 (dt, J = 2.8, 7.6 Hz, 1H), 6.85 (d, J = 8.8 Hz, 1H), | |
| | 5.10 (dd, J = 5.0, 13.2 Hz, 1H), 4.46-4.35 (m, 3H), 4.25 (d, J = 17.6 | |
| | Hz, 1H), 3.46-3.38 (m, 2H), 3.18-3.07 (m, 1H), 2.97-2.82 (m, 3H), | |
| | 2.80-2.70 (m, 3H), 2.68-2.55 (m, 2H), 2.46 (s, 3H), 2.45-2.36 (m, | |
| | 1H), 2.31-2.17 (m, 3H), 2.04-1.82 (m, 4H), 1.82-1.69 (m, 1H), | |
| | 1.41-1.27 (m, 2H), 0.77 ( d, J = 6.0 Hz, 3H) | |
| 65 | ¹H NMR (400 MHz, DMSO-d₆) δ 10.82 (s, 1H), 9.99 (s, 1H), 8.83 (s, | A |
| | 1H), 8.73 (d, J = 8.4 Hz, 1H), 8.45 (t, J = 6.4 Hz, 1H), 8.14 (s, 1H), 7.91 | |
| | (d, J = 2.0 Hz, 1H), 7.83 (s, 1H), 7.72 (d, J = 8.4 Hz, 1H), 7.61-7.49 | |
| | (m, 2H), 7.37 (dd, J = 2.0, 8.4Hz, 1H), 7.02-6.91 (m, 1H), 6.83 (d, J = | |
| | 8.4 Hz, 1H), 6.36 (d, J = 8.8 Hz, 1H), 4.39 (s, 2H), 4.04 (t, J = 8.0 Hz, | |
| | 2H), 3.73 (dd, J = 4.8, 12.4 Hz, 1H), 3.63-3.59 (m, 2H), 3.05 (d, J = | |
| | 10.8 Hz, 2H), 3.02-2.97 (m, 1H), 2.76 (d, J = 6.8 Hz, 2H), 2.71-2.63 | |
| | (m, 2H), 2.51 (s, 4H), 2.33-2.23 (m, 2H), 2.21-2.11 (m, 1H), 1.99- | |
| | 1.92 (m, 1H), 1.75-1.61 (m, 4H) | |
| 66 | ¹H NMR (400 MHz, DMSO-d₆) δ 10.89 (d, J = 12.8 Hz, 1H), 9.94 (s, | A |
| | 1H), 8.79 (s, 1H), 8.66 (d, J = 8.8 Hz, 1H), 8.47-8.41 (m, 1H), 8.13 (s, | |
| | 1H), 7.81 (s, 1H), 7.74-7.66 (m, 2H), 7.55-7.44 (m, 2H), 6.96 (d, J = | |
| | 2.4 Hz, 1H), 6.91-6.81 (m, 2H), 4.76-4.64 (m, 1H), 4.53-4.40 (m, | |
| | 4H), 4.38 (s, 2H), 3.02-2.92 (m, 3H), 2.90-2.79 (m, 5H), 2.61-2.58 | |
| | (m, 2H), 2.54 (s, 3H), 2.46 (s, 3H), 2.31 (s, 1H), 1.99 (s, 2H), 1.83 (d, J = | |
| | 12.8 Hz, 2H), 1.39 (dd, J = 6.8, 14.4 Hz, 3H), 1.19-1.07 (m, 2H) | |
| 67 | ¹H NMR (400 MHz, DMSO-d₆) δ 10.80 (s, 1H), 9.94 (s, 1H), 8.79 (s, | A |
| | 1H), 8.66 (d, J = 8.8 Hz, 1H), 8.46-8.41 (m, 1H), 7.90 (s, 1H), 7.81 (s, | |
| | 1H), 7.70 (d, J = 8.4 Hz, 1H), 7.54-7.43 (m, 2H), 7.37 (dd, J = 2.4, 8.4 | |
| | Hz, 1H), 6.96 (dt, J = 2.4, 7.2 Hz, 1H), 6.84 (d, J = 8.8 Hz, 1H), 6.36 (d, | |
| | J = 8.8 Hz, 1H), 4.38 (s, 2H), 4.03 (t, J = 7.6 Hz, 2H), 3.73 (dd, J = 4.8, | |
| | 12.4 Hz, 1H), 3.63-3.55 (m, 2H), 3.35 (s, 1H), 3.00-2.91 (m, 1H), | |
| | 2.84 (s, 4H), 2.65 (d, J = 7.2 Hz, 3H), 2.58-2.52 (m, 4H), 2.46 (s, 3H), | |
| | 2.17 (dd, J = 4.4, 12.4 Hz, 1H), 1.97 (d, J = 4.8 Hz, 1H) | |
| 68 | ¹H NMR (400 MHz, DMSO-d₆) δ 10.96 (s, 1H), 9.94 (s, 1H), 8.79 (s, | A |
| | 1H), 8.66 (d, J = 8.4 Hz, 1H), 8.44 (dd, J = 6.0, 7.2 Hz, 1H), 7.81 (s, | |
| | 1H), 7.70 (d, J = 8.4 Hz, 1H), 7.54-7.45 (m, 2H), 7.38 (d, J = 8.0 Hz, | |
| | 1H), 7.09 (d, J = 8.4 Hz, 1H), 6.96 (dt, J = 2.4, 7.2 Hz, 1H), 6.85 (d, J = | |
| | 8.4 Hz, 1H), 5.06 (dd, J = 4.8, 13.2 Hz, 1H), 4.44 (d, J = 16.8 Hz, 1H), | |
| | 4.38 (s, 2H), 4.27 (d, J = 16.8 Hz, 1H), 3.87 (s, 3H), 3.63-3.52 (m, | |
| | 2H), 2.96-2.84 (m, 5H), 2.74-2.52 (m, 8H), 2.47 (s, 3H), 2.45-2.36 | |
| | (m, 2H), 2.02-1.93 (m, 1H), 1.91-1.82 (m, 2H), 1.81-1.68 (m, 1H), | |
| | 1.40-1.29 (m, 2H) | |
| 69 | ¹H NMR (400 MHz, DMSO-d₆) δ 10.96 (s, 1H), 9.97 (s, 1H), 8.81 (s, | A |
| | 1H), 8.72 (d, J = 8.4 Hz, 1H), 8.44 (dd, J = 5.6, 7.6 Hz, 1H), 8.18 (s, | |
| | 1H), 7.82 (s, 1H), 7.71 (d, J = 8.4 Hz, 1H), 7.56-7.49 (m, 2H), 7.37 (d, | |
| | J = 8.0 Hz, 1H), 6.96 (dt, J = 2.4, 7.6 Hz, 1H), 6.81 (d, J = 8.4 Hz, 1H), | |
| | 6.58 (t, J = 8.0 Hz, 1H), 5.04 (dd, J = 5.2, 13.2 Hz, 1H), 4.46-4.37 (m, | |
| | 3H), 4.25 (d, J = 16.8 Hz, 1H), 4.10-4.05 (m, 2H), 3.95 (s, 2H), 2.99- | |
| | 2.85 (m, 4H), 2.66-2.59 (m, 2H), 2.56-2.54 (m, 1H), 2.43-2.29 (m, | |
| | 7H), 2.12-2.04 (m, 2H), 1.98-1.88 (m, 3H), 1.71-1.59 (m, 4H) | |
| 70 | ¹H NMR (400 MHz, DMSO-d₆) δ 10.99 (s, 1H), 9.98 (s, 1H), 8.80 (s, | A |
| | 1H), 8.73 (d, J = 8.4 Hz, 1H), 8.44 (dd, J = 6.0, 7.2 Hz, 1H), 7.82 (s, | |
| | 1H), 7.71 (d, J = 8.4 Hz, 1H), 7.55-7.47 (m, 2H), 7.46-7.40 (m, 1H), | |
| | 7.29 (d, J = 7.2 Hz, 1H), 7.16 (d, J = 8.0 Hz, 1H), 6.96 (dt, J = 2.8, 7.6 | |
| | Hz, 1H), 6.82 (d, J = 8.4 Hz, 1H), 5.12 (dd, J = 4.8, 13.2 Hz, 1H), 4.47- | |
| | 4.26 (m, 4H), 3.48-3.35 (m, 3H), 2.97-2.71 (m, 9H), 2.63-2.52 (m, | |
| | 5H), 2.27 ( d, J = 6.8 Hz, 2H), 2.05-1.94 (m, 1H), 1.91-1.79 (m, 2H), | |
| | 1.79-1.64 (m, 1H), 1.35-1.23 (m, 5H) | |
| 71 | ¹H NMR (400 MHz, DMSO-d₆) δ 11.09 (s, 1H), 9.96 ( s, 1H), 8.80 (s, | A |
| | 1H), 8.66 (d, J = 8.4 Hz, 1H), 8.49-8.40 (m, 1H), 7.82 (s, 1H), 7.71 (d, | |
| | J = 8.4 Hz, 1H), 7.56-7.43 (m, 2H), 7.03-6.82 (m, 5H), 5.41-5.29 | |
| | (m, 1H), 4.38 (s, 2H), 3.64 (s, 3H), 3.40-3.34 (m, 2H), 3.19-3.10 (m, | |
| | 3H), 2.98-2.83 (m, 4H), 2.76-2.58 (m, 6H), 2.55-2.51 (m, 3H), 2.48 | |
| | (s, 3H), 2.03-1.96 (m, 1H), 1.91-1.83 (m, 2H), 1.50-1.31 (m, 2H) | |
| 72 | ¹H NMR (400 MHz, DMSO-d₆) δ 11.07 (s, 1H), 9.94 (s, 1H), 8.79 (s, | A |
| | 1H), 8.66 (d, J = 8.8 Hz, 1H), 8.44 (dd, J = 6.0, 7.2 Hz, 1H), 8.15 (s, | |
| | 1H), 7.81 (s, 1H), 7.70 (d, J = 8.4 Hz, 1H), 7.52 (dd, J = 2.4, 10.0 Hz, | |
| | 1H), 7.47 (d, J = 8.8 Hz, 1H), 7.00-6.93 (m, 2H), 6.89 (d, J = 7.6 Hz, | |
| | 1H), 6.86-6.80 (m, 2H), 5.68-5.56 (m, 1H), 5.29 ( dd, J = 5.2, 12.8 | |
| | Hz, 1H), 4.38 (s, 2H), 3.11 ( d, J = 11.6 Hz, 2H), 2.85 ( s, 5H), 2.75- | |
| | 2.68 (m, 2H), 2.66-2.52 (m, 6H), 2.46 (s, 3H), 2.29 ( d, J = 6.8 Hz, | |
| | 2H), 2.02-1.93 (m, 1H), 1.87 ( d, J = 12.4 Hz, 2H), 1.75-1.63 (m, | |
| | 1H), 1.47 (dd, J = 6.8, 10.8 Hz, 6H), 1.34-1.20 (m, 2H) | |
| 73 | ¹H NMR (400 MHz, DMSO-d₆) δ 11.08 (s, 1H), 10.02 (s, 1H), 8.83 (s, | A |
| | 1H), 8.74 (d, J = 8.8 Hz, 1H), 8.44 (dd, J = 6.0, 7.2 Hz, 1H), 7.83 (s, | |
| | 1H), 7.73 (d, J = 8.8 Hz, 1H), 7.54 (d, J = 2.4 Hz, 1H), 7.52 (d, J = 2.4 | |
| | Hz, 1H), 7.01-6.95 (m, 2H), 6.92-6.82 (m, 3H), 5.62 (s, 1H), 5.34- | |
| | 5.26 (m, 1H), 4.39 (s, 2H), 3.14 (d, J = 9.6 Hz, 2H), 2.94-2.82 (m, 3H), | |
| | 2.77-2.69 (m, 3H), 2.65-2.57 (m, 3H), 2.55-2.52 (m, 6H), 2.05- | |
| | 1.74 (m, 9H), 1.49 (dd, J = 6.8, 11.2 Hz, 6H), 1.42-1.29 (m, 2H) | |
| 74 | ¹H NMR (400 MHz, DMSO-d₆) δ 10.99-10.94 (m, 1H), 10.00-9.95 | A |
| | (m, 1H), 8.81 (s, 1H), 8.73 (d, J = 8.4 Hz, 1H), 8.47-8.42 (m, 1H), 7.82 | |
| | (s, 1H), 7.72 (d, J = 8.8 Hz, 1H), 7.58-7.50 (m, 2H), 7.38 (d, J = 8.0 | |
| | Hz, 1H), 7.09 (d, J = 8.0 Hz, 1H), 6.96 (dt, J = 2.4, 7.6 Hz, 1H), 6.82 (d, | |
| | J = 8.4 Hz, 1H), 5.06 (dd, J = 5.2, 13.2 Hz, 1H), 4.47-4.37 (m, 3H), | |
| | 4.26 (d, J = 16.8 Hz, 1H), 3.87 (s, 3H), 3.61-3.52 (m, 2H), 3.00 (d, J = | |
| | 9.6 Hz, 2H), 2.89 (d, J = 12.4 Hz, 1H), 2.69 (s, 1H), 2.65-2.56 (m, | |
| | 6H), 2.46-2.43 (m, 1H), 2.26 (d, J = 6.8 Hz, 2H), 2.10-2.01 (m, 2H), | |
| | 2.00-1.93 (m, 1H), 1.90-1.82 (m, 2H), 1.76-1.60 (m, 5H), 1.39- | |
| | 1.24 (m, 2H) | |
| 75 | ¹H NMR (400 MHz, DMSO-d₆) δ 10.98 (s, 1H), 9.88 (s, 1H), 8.78 (s, | A |
| | 1H), 8.49 (d, J = 8.4 Hz, 1H), 8.42 (dd, J = 6.0, 7.2 Hz, 1H), 8.17 (s, | |
| | 1H), 7.81 (s, 1H), 7.70 (d, J = 8.4 Hz, 1H), 7.64 (d, J = 8.0 Hz, 1H), | |
| | 7.51 (dd, J = 2.4, 10.0 Hz, 1H), 7.26 (dd, J = 1.6, 8.4 Hz, 2H), 6.97 (dt, | |
| | J = 2.8, 7.6 Hz, 1H), 6.55 (d, J = 8.0 Hz, 1H), 5.09 (dd, J = 5.2, 13.2 Hz, | |
| | 1H), 4.44-4.34 (m, 3H), 4.25 (d, J = 17.2 Hz, 1H), 3.99 (s, 3H), 3.83- | |
| | 3.63 (m, 2H), 3.05-2.84 (m, 6H), 2.74 (t, J = 11.2 Hz, 2H), 2.65-2.53 | |
| | (m, 3H), 2.47-2.38 (m, 2H), 2.27 (d, J = 6.8 Hz, 2H), 2.03-1.95 (m, | |
| | 1H), 1.86 (d, J = 11.6 Hz, 2H), 1.72 (d, J = 7.2 Hz, 1H), 1.40-1.26 (m, | |
| | 2H) | |
| 76 | ¹H NMR (400 MHz, DMSO-d₆) δ 11.06 (s, 1H), 9.94 (s, 1H), 8.79 (s, | A |
| | 1H), 8.65 (d, J = 8.8 Hz, 1H), 8.44 (dd, J = 6.0, 7.2 Hz, 1H), 7.81 (s, | |
| | 1H), 7.70 (d, J = 8.4 Hz, 1H), 7.52 (dd, J = 2.4, 10.0 Hz, 1H), 7.46 (d, | |
| | J = 8.8 Hz, 1H), 6.99-6.91 (m, 2H), 6.87-6.81 (m, 2H), 6.63 (dd, J = | |
| | 2.0, 8.4 Hz, 1H), 5.35-5.23 (m, 1H), 4.38 (s, 2H), 3.58 (d, J = 11.6 Hz, | |
| | 2H), 3.30 (s, 3H), 2.95-2.87 (m, 1H), 2.84 (s, 4H), 2.68-2.53 (m, 8H), | |
| | 2.46 (s, 3H), 2.42 (t, J = 6.4 Hz, 2H), 2.02-1.94 (m, 1H), 1.79 (d, J = | |
| | 12.0 Hz, 2H), 1.45 (s, 3H), 1.38-1.26 (m, 2H) | |
| 77 | ¹H NMR (400 MHz, DMSO-d₆) δ 11.06 (s, 1H), 9.98 (s, 1H), 8.81 (s, | A |
| | 1H), 8.73 (d, J = 8.8 Hz, 1H), 8.47-8.41 (m, 1H), 8.18 (s, 1H), 7.82 (s, | |
| | 1H), 7.72 (d, J = 8.4 Hz, 1H), 7.57-7.49 (m, 2H), 7.01-6.90 (m, 2H), | |
| | 6.85-6.79 (m, 2H), 6.63 (dd, J = 2.0, 8.4 Hz, 1H), 5.29 (dd, J = 5.4, | |
| | 12.8 Hz, 1H), 4.38 (s, 2H), 3.58 (br d, J = 12.1 Hz, 2H), 3.30 (s, 3H), | |
| | 3.06 (d, J = 11.6 Hz, 2H), 2.87 (d, J = 18.0 Hz, 1H), 2.72-2.57 (m, | |
| | 8H), 2.47-2.43 (m, 2H), 2.11 (t, J = 10.8 Hz, 2H), 2.01-1.94 (m, 1H), | |
| | 1.79 (d, J = 12.0 Hz, 2H), 1.74-1.61 (m, 4H), 1.52-1.40 (m, 3H), | |
| | 1.38-1.27 (m, 2H) | |
| 78 | ¹H NMR (400 MHz, DMSO-d₆) δ 10.99 (s, 1H), 9.98 (s, 1H), 8.81 (s, | A |
| | 1H), 8.73 (d, J = 8.8 Hz, 1H), 8.49-8.39 (m, 1H), 8.15 (s, 1H), 7.82 (s, | |
| | 1H), 7.72 (d, J = 8.4 Hz, 1H), 7.68 (s, 1H), 7.56 (d, J = 8.0 Hz, 1H), | |
| | 7.52 (dd, J = 2.4, 10.0 Hz, 1H), 7.36 (s, 1H), 6.96 (dt, J = 2.4, 7.2 Hz, | |
| | 1H), 6.82 (d, J = 8.4 Hz, 1H), 5.12-5.03 (m, 1H), 4.45-4.35 (m, 3H), | |
| | 4.32-4.23 (m, 1H), 3.45-3.38 (m, 2H), 3.00 ( d, J = 9.6 Hz, 2H), | |
| | 2.96-2.84 (m, 1H), 2.77-2.64 (m, 3H), 2.63-2.57 (m, 1H), 2.53-2.51 (m, | |
| | 3H), 2.38-2.31 (m, 1H), 2.27 ( d, J = 7.2 Hz, 2H), 2.12-1.96 (m, 3H), | |
| | 1.91-1.81 (m, 2H), 1.78-1.57 (m, 5H), 1.41-1.26 (m, 2H) | |
| 79 | ¹H NMR (400 MHz, DMSO-d₆) δ 10.96 (s, 1H), 9.93 (s, 1H), 8.78 (s, | A |
| | 1H), 8.65 (d, J = 8.8 Hz, 1H), 8.44 (t, J = 6.8 Hz, 1H), 7.81 (s, 1H), 7.70 | |
| | (d, J = 8.4 Hz, 1H), 7.54-7.45 (m, 2H), 7.41 (d, J = 8.4 Hz, 1H), 7.28- | |
| | 7.23 (m, 1H), 7.16 (d, J = 1.6 Hz, 1H), 6.96 (dt, J = 2.8, 7.6 Hz, 1H), | |
| | 6.84 (d, J = 8.8 Hz, 1H), 5.09 (dd, J = 4.8, 13.2 Hz, 1H), 4.40-4.28 (m, | |
| | 3H), 4.24-4.17 (m, 1H), 3.76 (d, J = 11.6 Hz, 2H), 2.96-2.89 (m, 1H), | |
| | 2.85 (s, 4H), 2.73 (t, J = 11.6 Hz, 2H), 2.61 (s, 1H), 2.55 (d, J = 6.8 Hz, | |
| | 4H), 2.46 (s, 3H), 2.41-2.35 (m, 1H), 2.25 (d, J = 6.8 Hz, 2H), 2.03- | |
| | 1.95 (m, 1H), 1.87-1.80 (m, 2H), 1.79-1.69 (m, 1H), 1.32-1.19 (m, | |
| | 2H) | |
| 80 | ¹H NMR (400 MHz, DMSO-d₆) δ 10.95 (s, 1H), 9.93 (s, 1H), 8.78 (s, | A |
| | 1H), 8.65 (d, J = 8.4 Hz, 1H), 8.46-8.41 (m, 1H), 8.17 (s, 1H), 7.81 (s, | |
| | 1H), 7.70 (d, J = 8.4 Hz, 1H), 7.51 (dd, J = 2.4, 10.0 Hz, 1H), 7.46 (d, J = | |
| | 8.8 Hz, 1H), 7.37 (d, J = 8.0 Hz, 1H), 6.96 (dt, J = 2.4, 7.6 Hz, 1H), | |
| | 6.83 (d, J = 8.8 Hz, 1H), 6.58 (t, J = 8.0 Hz, 1H), 5.04 (dd, J = 4.8, 13.2 | |
| | Hz, 1H), 4.43 (d, J = 16.8 Hz, 1H), 4.38 (s, 2H), 4.26 (d, J = 16.4 Hz, | |
| | 1H), 4.08 (s, 2H), 3.95 (s, 2H), 2.94-2.89 (m, 1H), 2.82 (s, 5H), 2.60 | |
| | (s, 1H), 2.55 (s, 2H), 2.45 (s, 4H), 2.41 (s, 3H), 2.39-2.30 (m, 3H), | |
| | 1.99-1.87 (m, 3H) | |
| 81 | ¹H NMR (400 MHz, DMSO-d₆) δ 10.98 (s, 1H), 9.94 (s, 1H), 8.79 (s, | A |
| | 1H), 8.65 (d, J = 8.8 Hz, 1H), 8.44 (dd, J = 6.0, 7.6 Hz, 1H), 7.81 (s, | |
| | 1H), 7.71 (d, J = 8.4 Hz, 1H), 7.52 (dd, J = 2.4, 10.0 Hz, 1H), 7.48- | |
| | 7.40 (m, 2H), 7.30 (d, J = 7.2 Hz, 1H), 7.17 (d, J = 7.6 Hz, 1H), 6.96 | |
| | (dt, J = 2.4, 7.6 Hz, 1H), 6.84 (d, J = 7.6 Hz, 1H), 5.12 (dd, J = 4.8, 13.2 | |
| | Hz, 1H), 4.47-4.40 (m, 1H), 4.38 (s, 2H), 4.34-4.26 (m, 1H), 3.42- | |
| | 3.35 (m, 5H), 2.93-2.82 (m, 3H), 2.73 (d, J = 9.2 Hz, 2H), 2.65-2.54 | |
| | (m, 6H), 2.27 (dd, J = 1.6, 3.6 Hz, 2H), 2.03-1.97 (m, 1H), 1.92-1.81 | |
| | (m, 2H), 1.63-1.52 (m, 1H), 1.30-1.22 (m, 2H), 1.10 (s, 6H) | |
| 82 | ¹H NMR (400 MHz, DMSO-d₆) δ 11.00 (s, 1H), 10.05 (s, 1H), 8.87- | A |
| | 8.75 (m, 2H), 8.44 (dd, J = 6.0, 7.2 Hz, 1H), 7.83 (s, 1H), 7.73 (d, J = | |
| | 8.4 Hz, 1H), 7.68-7.61 (m, 2H), 7.53 (dd, J = 2.4, 10.0 Hz, 1H), 7.28 | |
| | (d, J = 8.4 Hz, 1H), 6.97 (d, J = 2.8 Hz, 1H), 6.90 (d, J = 8.4 Hz, 1H), | |
| | 5.14-5.04 (m, 3H), 4.45-4.38 (m, 3H), 4.26 (d, J = 17.6 Hz, 1H), 3.45- | |
| | 3.38 (m, 3H), 3.05 (s, 3H), 2.94-2.87 (m, 1H), 2.78 (d, J = 12.0 Hz, | |
| | 2H), 2.61 (s, 1H), 2.58 (s, 1H), 2.54 (s, 1H), 2.43 (d, J = 4.8 Hz, 1H), | |
| | 2.32 (s, 1H), 2.17 (s, 1H), 2.00 (d, J = 5.4 Hz, 1H), 1.88 (d, J = 10.8 Hz, | |
| | 2H), 1.77-1.68 (m, 4H), 1.49 (d, J = 5.4 Hz, 3H), 1.35 (d, J = 10.8 Hz, | |
| | 2H) | |
| 83 | ¹H NMR (400 MHz, DMSO-d₆) δ 11.09 (s, 1H), 9.98 (s, 1H), 8.81 (s, | A |
| | 1H), 8.70 (d, J = 8.4 Hz, 1H), 8.42 (dd, J = 6.4, 7.2 Hz, 1H), 8.14-8.11 | |
| | (m, 1H), 7.81 (s, 1H), 7.71 (d, J = 8.4 Hz, 1H), 7.52 (dd, J = 2.4, 10.0 | |
| | Hz, 1H), 7.00-6.94 (m, 2H), 6.92-6.82 (m, 3H), 5.35 (dd, J = 5.2, | |
| | 12.4 Hz, 1H), 4.38 (s, 2H), 3.64 (s, 3H), 3.14 (d, J = 10.0 Hz, 3H), | |
| | 2.95-2.83 (m, 2H), 2.75-2.70 (m, 3H), 2.69-2.66 (m, 4H), 2.64 (s, 2H), | |
| | 2.33 (s, 3H), 1.99 (dd, J = 4.4, 9.2 Hz, 2H), 1.86 (d, J = 12.4 Hz, 2H), | |
| | 1.78 (s, 4H), 1.36 (d, J = 12.4 Hz, 2H) | |
| 84 | ¹H NMR (400 MHz, DMSO-d₆) δ 11.11-11.05 (m, 1H), 10.12 (s, 1H), | A |
| | 9.01 (d, J = 8.4 Hz, 1H), 8.78 (s, 1H), 8.48-8.41 (m, 1H), 8.26 (s, 1H), | |
| | 7.82 (s, 1H), 7.73 (d, J = 8.4 Hz, 1H), 7.52 (dd, J = 2.8, 10.0 Hz, 1H), | |
| | 7.45 (s, 1H), 7.00-6.93 (m, 2H), 6.93-6.84 (m, 2H), 5.38-5.31 (m, | |
| | 1H), 4.40 (s, 2H), 3.64 (s, 3H), 3.18-3.07 (m, 3H), 3.02-2.96 (m, 2H), | |
| | 2.91-2.83 (m, 1H), 2.74-2.58 (m, 5H), 2.54 (s, 1H), 2.31-2.22 (m, | |
| | 5H), 2.08-1.97 (m, 3H), 1.86 (d, J = 11.6 Hz, 2H), 1.75-1.58 (m, 6H), | |
| | 1.41-1.27 (m, 2H) | |
| 85 | ¹H NMR (400 MHz, DMSO-d₆) δ 11.10 (s, 1H), 10.20 (s, 1H), 8.92 (d, | A |
| | J = 8.8 Hz, 1H), 8.84 (s, 1H), 8.44 (t, J = 6.6 Hz, 1H), 8.08 (s, 1H), 7.84 | |
| | (s, 1H), 7.72 (d, J = 8.4 Hz, 1H), 7.56-7.48 (m, 2H), 7.02-6.96 (m, | |
| | 2H), 6.93-6.84 (m, 2H), 5.36 (dd, J = 5.2, 12.8 Hz, 1H), 4.40 (s, 2H), | |
| | 3.64 (s, 3H), 3.16 (d, J = 9.6 Hz, 4H), 3.00-2.80 (m, 2H), 2.76-2.56 | |
| | (m, 7H), 2.52 (s, 1H), 2.36 (s, 3H), 2.04-1.96 (m, 1H), 1.88 (d, J = 9.6 | |
| | Hz, 7H), 1.52-1.32 (m, 2H) | |
| 86 | ¹H NMR (400 MHz, DMSO-d₆) δ 10.98 (s, 1H), 9.98 (s, 1H), 8.80 (s, | A |
| | 1H), 8.71 (d, J = 8.8 Hz, 1H), 8.43 (t, J = 6.4 Hz, 1H), 8.29 (s, 1H), 7.81 | |
| | (s, 1H), 7.71 (d, J = 8.4 Hz, 1H), 7.65 (d, J = 8.4 Hz, 1H), 7.56-7.49 | |
| | (m, 2H), 7.28 (d, J = 8.0 Hz, 1H), 7.01-6.93 (m, 1H), 6.56 (d, J = 8.0 | |
| | Hz, 1H), 5.10 (dd, J = 4.8, 13.2 Hz, 1H), 4.45-4.35 (m, 3H), 4.29- | |
| | 4.22 (m, 1H), 2.99 (d, J = 10.0 Hz, 2H), 2.91 (s, 1H), 2.81 (s, 6H), 2.79- | |
| | 2.71 (m, 3H), 2.67 (s, 1H), 2.62-2.55 (m, 3H), 2.33 (s, 1H), 2.25 (d, | |
| | J = 6.4 Hz, 2H), 2.04-1.96 (m, 3H), 1.91-1.84 (m, 2H), 1.70-1.63 (m, | |
| | 4H), 1.37-1.28 (m, 2H) | |
| 87 | ¹H NMR (400 MHz, DMSO-d₆) δ 11.01-10.94 (m, 1H), 9.93 (s, 1H), | A |
| | 8.78 (s, 1H), 8.68-8.62 (m, 1H), 8.48-8.41 (m, 1H), 8.29 (d, J = 2.4 | |
| | Hz, 1H), 7.81 (s, 1H), 7.70 (d, J = 8.4 Hz, 1H), 7.51 (dd, J = 2.4, 10.0 | |
| | Hz, 1H), 7.46-7.41 (m, 2H), 7.30 (d, J = 7.2 Hz, 1H), 7.17 (d, J = 7.6 | |
| | Hz, 1H), 6.96 (dt, J = 2.4, 7.2 Hz, 1H), 6.84 (d, J = 8.4 Hz, 1H), 5.12 | |
| | (dd, J = 5.2, 13.2 Hz, 1H), 4.47-4.36 (m, 3H), 4.33-4.26 (m, 1H), | |
| | 3.42 (s, 2H), 2.98-2.83 (m, 5H), 2.81-2.66 (m, 4H), 2.57 (s, 1H), 2.47 | |
| | (s, 3H), 2.41-2.31 (m, 2H), 2.14-2.07 (m, 1H), 2.02-1.91 (m, 2H), | |
| | 1.81-1.75 (m, 1H), 1.72-1.62 (m, 1H), 1.34-1.23 (m, 2H), 1.08 (d, | |
| | J = 5.6 Hz, 3H) | |
| 88 | ¹H NMR (400 MHz, DMSO-d₆) δ 11.08 (s, 1H), 9.93 (s, 1H), 8.78 (s, | A |
| | 1H), 8.65 (d, J = 8.4 Hz, 1H), 8.44 (dd, J = 6.0, 7.2 Hz, 1H), 7.81 (s, | |
| | 1H), 7.70 (d, J = 8.4 Hz, 1H), 7.54-7.45 (m, 2H), 7.00-6.93 (m, 2H), | |
| | 6.93-6.82 (m, 3H), 5.34 (dd, J = 5.6, 12.8 Hz, 1H), 4.38 (s, 2H), 3.63 | |
| | (s, 3H), 3.10 (d, J = 10.4 Hz, 2H), 2.91-2.80 (m, 5H), 2.75-2.63 (m, | |
| | 4H), 2.57-2.52 (m, 3H), 2.48-2.38 (m, 6H), 2.04-1.94 (m, 1H), 1.80 | |
| | (d, J = 11.2 Hz, 2H), 1.54-1.33 (m, 5H) | |
| 89 | ¹H NMR (400 MHz, DMSO-d₆) δ 11.08 (s, 1H), 9.98 (s, 1H), 8.80 (s, | A |
| | 1H), 8.73 (d, J = 8.8 Hz, 1H), 8.44 (dd, J = 6.0, 7.2 Hz, 1H), 7.82 (s, | |
| | 1H), 7.71 (d, J = 8.8 Hz, 1H), 7.56 (d, J = 8.4 Hz, 1H), 7.52 (dd, J = 2.8, | |
| | 10.0 Hz, 1H), 7.00-6.93 (m, 2H), 6.93-6.80 (m, 3H), 5.35 (dd, J = | |
| | 5.2, 12.4 Hz, 1H), 4.38 (s, 2H), 3.63 (s, 3H), 3.13-3.00 (m, 5H), 2.93- | |
| | 2.84 (m, 1H), 2.75-2.53 (m, 6H), 2.41 (t, J = 6.8 Hz, 3H), 2.08-1.96 | |
| | (m, 3H), 1.80 (d, J = 10.4 Hz, 2H), 1.73-1.60 (m, 4H), 1.53-1.35 (m, | |
| | 5H) | |
| 90 | ¹H NMR (400 MHz, MeOD-d₄) δ 8.86 (d, J = 8.4 Hz, 1H), 8.31 (dd, J = | A |
| | 5.8, 7.6 Hz, 1H), 7.76-7.63 (m, 3H), 7.33 (dd, J = 2.2, 9.6 Hz, 1H), | |
| | 7.11-6.87 (m, 5H), 5.38-5.28 (m, 1H), 5.17 (s, 1H), 4.94 (br d, J = 5.2 | |
| | Hz, 3H), 4.82-4.75 (m, 1H), 4.39 (s, 2H), 3.77 (s, 3H), 3.74-3.61 (m, | |
| | 2H), 3.24 (br s, 2H), 3.16-3.06 (m, 3H), 2.95-2.77 (m, 5H), 2.20- | |
| | 2.13 (m, 1H), 2.12-2.03 (m, 4H), 2.00-1.93 (m, 2H), 1.72-1.56 (m, | |
| | 2H) | |
| 91 | ¹H NMR (400 MHz, DMSO-d₆) δ 10.97 (s, 1H), 10.02 (s, 1H), 8.81 (s, | A |
| | 1H), 8.72 (d, J = 8.4 Hz, 1H), 8.46-8.39 (m, 1H), 7.82 (s, 1H), 7.72 (d, | |
| | J = 8.4 Hz, 1H), 7.52 (dd, J = 2.4, 10.0 Hz, 2H), 7.43 (d, J = 8.4 Hz, | |
| | 1H), 7.30-7.24 (m, 1H), 7.19 (s, 1H), 6.97 (dt, J = 2.8, 7.6 Hz, 1H), | |
| | 6.62 (d, J = 8.4 Hz, 1H), 5.10 (dd, J = 5.2, 13.2 Hz, 1H), 4.42-4.30 (m, | |
| | 3H), 4.24-4.16 (m, 1H), 3.80 (d, J = 10.4 Hz, 2H), 3.00-2.86 (m, 3H), | |
| | 2.82 (s, 6H), 2.81-2.70 (m, 3H), 2.70-2.60 (m, 2H), 2.56 (d, J = 17.6 | |
| | Hz, 2H), 2.38 (dd, J = 4.0, 12.8 Hz, 2H), 2.06-1.94 (m, 2H), 1.91- | |
| | 1.76 (m, 6H), 1.38-1.26 (m, 2H) | |
| 92 | ¹H NMR (400 MHz, DMSO-d₆) δ 11.05 (s, 1H), 9.99 (s, 1H), 8.81 (s, | A |
| | 1H), 8.73 (d, J = 8.4 Hz, 1H), 8.49-8.39 (m, 1H), 7.82 (s, 1H), 7.72 (d, | |
| | J = 8.4 Hz, 1H), 7.58-7.45 (m, 2H), 6.96 (dt, J = 2.4, 7.6 Hz, 1H), 6.89 | |
| | (d, J = 8.4 Hz, 1H), 6.83 (d, J = 8.0 Hz, 1H), 6.29 (d, J = 1.6 Hz, 1H), | |
| | 6.09 (dd, J = 1.6, 8.4 Hz, 1H), 5.33-5.17 (m, 1H), 4.39 (s, 2H), 3.55- | |
| | 3.45 (m, 4H), 3.28 (s, 3H), 3.13-3.00 (m, 2H), 2.96-2.84 (m, 1H), | |
| | 2.75-2.57 (m, 3H), 2.53-2.51 (m, 3H), 2.31-2.09 (m, 3H), 2.02- | |
| | 1.94 (m, 1H), 1.90 ( d, J = 11.6 Hz, 2H), 1.72 ( s, 6H), 1.61-1.43 (m, | |
| | 3H), 1.23 ( s, 1H), 1.05-0.91 (m, 2H) | |
| 93 | ¹H NMR (400 MHz, DMSO-d₆) δ 10.99 (s, 1H), 9.92 (s, 1H), 8.77 (s, | C |
| | 1H), 8.65 (d, J = 8.8 Hz, 1H), 8.46-8.41 (m, 1H), 8.22 (s, 1H), 7.81 (s, | |
| | 1H), 7.70 (d, J = 8.8 Hz, 1H), 7.60-7.42 (m, 5H), 6.96 (dt, J = 2.4, 7.5 | |
| | Hz, 1H), 6.84 (d, J = 8.8 Hz, 1H), 5.14 (dd, J = 5.2, 13.2 Hz, 1H), 4.53 | |
| | (d, J = 17.2 Hz, 1H), 4.41-4.32 (m, 3H), 3.00 (t, J = 9.6 Hz, 5H), 2.68- | |
| | 2.56 (m, 5H), 2.45 (s, 3H), 2.25 (d, J = 7.2 Hz, 2H), 2.06-1.99 (m, 3H), | |
| | 1.84 (d, J = 12.4 Hz, 2H), 1.76 (s, 5H), 1.33-1.23 (m, 2H) | |
| 94 | ¹H NMR (400 MHz, DMSO-d₆) δ 11.04 (s, 1H), 10.00 (s, 1H), 8.80 (s, | A |
| | 1H), 8.68 (d, J = 8.4 Hz, 1H), 8.44 (dd, J = 6.0, 7.2 Hz, 1H), 7.80 (s, | |
| | 1H), 7.72 (d, J = 8.4 Hz, 1H), 7.56-7.48 (m, 2H), 7.00-6.92 (m, 2H), | |
| | 6.88-6.84 (m, 2H), 6.68-6.64 (m, 1H), 5.32 (dd, J = 5.6, 12.8 Hz, | |
| | 1H), 4.40 (s, 2H), 3.64 (d, J = 11.2 Hz, 3H), 3.32 (s, 3H), 3.28-2.80 | |
| | (m, 8H), 2.76-2.56 (m, 5H), 2.48-2.44 (m, 4H), 2.04-1.80 (m, 4H), | |
| | 1.48-1.28 (m, 2H) | |
| 95 | ¹H NMR (400 MHz, DMSO-d₆) δ 11.06 (s, 1H), 9.98 (s, 1H), 8.81 (s, | A |
| | 1H), 8.73 (d, J = 8.4 Hz, 1H), 8.45 (dd, J = 6.0, 7.2 Hz, 1H), 8.15 (s, | |
| | 1H), 7.82 (s, 1H), 7.72 (d, J = 8.4 Hz, 1H), 7.59-7.49 (m, 2H), 7.00- | |
| | 6.89 (m, 2H), 6.85-6.78 (m, 2H), 6.64 (dd, J = 2.0, 8.8 Hz, 1H), 5.34- | |
| | 5.23 (m, 1H), 4.39 (s, 2H), 3.66-3.51 (m, 2H), 3.33-3.32 (m, 3H), | |
| | 3.00 ( d, J = 10.4 Hz, 2H), 2.71-2.56 (m, 5H), 2.48-2.46 (m, 3H), | |
| | 2.28-2.20 (m, 2H), 2.11-1.92 (m, 3H), 1.88-1.78 (m, 2H), 1.75- | |
| | 1.59 (m, 5H), 1.35-1.18 (m, 2H) | |
| 96 | ¹H NMR (400 MHz, DMSO-d₆) δ 10.94 (s, 1H), 9.97 (s, 1H), 8.80 (s, | A |
| | 1H), 8.72 (d, J = 8.8 Hz, 1H), 8.47-8.41 (m, 1H), 7.82 (s, 1H), 7.71 (d, | |
| | J = 8.4 Hz, 1H), 7.58-7.48 (m, 2H), 6.96 (dt, J = 2.4, 7.6 Hz, 1H), 6.82 | |
| | (d, J = 8.4 Hz, 1H), 6.33 (d, J = 8.0 Hz, 1H), 4.99 (dd, J = 5.2, 13.2 Hz, | |
| | 1H), 4.41-4.32 (m, 3H), 4.19 (d, J = 16.8 Hz, 1H), 4.06 (s, 2H), 3.93 | |
| | (s, 2H), 2.98-2.83 (m, 4H), 2.69-2.59 (m, 4H), 2.54 (s, 3H), 2.45- | |
| | 2.27 (m, 6H), 2.10-2.00 (m, 2H), 1.98-1.85 (m, 3H), 1.73-1.54 (m, | |
| | 4H) | |
| 97 | ¹H NMR (400 MHz, MeOD-d₄) δ 8.76 (d, J = 8.4 Hz, 1H), 8.44-8.36 | A |
| | (m, 1H), 8.31 (dd, J = 5.6, 7.4 Hz, 1H), 7.71-7.63 (m, 2H), 7.37-7.29 | |
| | (m, 1H), 7.05 (td, J = 7.6, 15.2 Hz, 2H), 6.97-6.88 (m, 2H), 6.69 (s, | |
| | 1H), 5.38-5.28 (m, 1H), 4.57 (br s, 4H), 4.37 (s, 2H), 3.77 (s, 3H), | |
| | 3.73-3.66 (m, 2H), 3.27-3.24 (m, 2H), 3.18-3.04 (m, 4H), 2.97-2.73 (m, | |
| | 6H), 2.66-2.64 (m, 3H), 2.53 (br d, J = 11.2 Hz, 2H), 2.46 (s, 3H), | |
| | 2.22-2.03 (m, 2H), 1.96 (br d, J = 10.8 Hz, 4H), 1.69-1.56 (m, 2H) | |
| 98 | ¹H NMR (400 MHz, MeOD-d₄) δ 8.89 (d, J = 8.4 Hz, 1H), 8.32 (dd, J = | A |
| | 5.6, 7.4 Hz, 1H), 7.75-7.60 (m, 3H), 7.34 (dd, J = 2.4, 9.2 Hz, 1H), | |
| | 7.03-6.92 (m, 3H), 6.90 (d, J = 2.0 Hz, 1H), 6.82 (dd, J = 2.2, 8.4 Hz, | |
| | 1H), 5.29 (dd, J = 5.4, 12.4 Hz, 1H), 4.90 (s, 2H), 4.83 (s, 2H), 4.39 (s, | |
| | 2H), 3.83-3.71 (m, 2H), 3.68 (br d, J = 12.0 Hz, 2H), 3.43-3.39 (m, | |
| | 3H), 3.25-3.11 (m, 4H), 2.95-2.86 (m, 1H), 2.85-2.75 (m, 4H), | |
| | 2.21-2.03 (m, 6H), 2.01-1.93 (m, 2H), 1.65-1.50 (m, 2H) | |
| 99 | ¹H NMR (400 MHz, DMSO-d₆) δ 11.05 (s, 1H), 9.98 (s, 1H), 8.81 (s, | A |
| | 1H), 8.73 (d, J = 8.8 Hz, 1H), 8.44 (dd, J = 6.0, 7.6 Hz, 1H), 7.82 (s, | |
| | 1H), 7.72 (d, J = 8.8 Hz, 1H), 7.58-7.49 (m, 2H), 6.96 (dt, J = 2.8, 7.6 | |
| | Hz, 1H), 6.90 (d, J = 8.4 Hz, 1H), 6.83 (d, J = 8.4 Hz, 1H), 6.31 (d, J = | |
| | 2.0 Hz, 1H), 6.13-6.08 (m, 1H), 5.33-5.21 (m, 1H), 4.39 (s, 2H), 3.94 | |
| | (t, J = 7.2 Hz, 2H), 3.45 ( t, J = 6.4 Hz, 2H), 3.28 (s, 3H), 3.02-2.86 | |
| | (m, 4H), 2.70-2.60 (m, 5H), 2.52-2.51 (m, 3H), 2.21-2.09 (m, 2H), | |
| | 2.01-1.93 (m, 1H), 1.74-1.61 (m, 4H) | |
| 100 | ¹H NMR (400 MHz, DMSO-d₆) δ 11.07 (s, 1H), 9.79 (s, 1H), 8.73 (s, | A |
| | 1H), 8.56 (d, J = 8.8 Hz, 1H), 8.42 (dd, J = 6.0, 7.2 Hz, 1H), 8.16 (s, | |
| | 1H), 7.80 (s, 1H), 7.67 (d, J = 8.8 Hz, 1H), 7.51 (dd, J = 2.4, 10.0 Hz, | |
| | 1H), 7.24 (d, J = 8.8 Hz, 1H), 7.00-6.92 (m, 2H), 6.87 (dd, J = 7.6, | |
| | 13.2 Hz, 2H), 6.79 (d, J = 8.8 Hz, 1H), 5.34 (dd, J = 5.2, 12.8 Hz, 1H), | |
| | 4.36 (s, 2H), 3.96 (s, 1H), 3.61 (s, 3H), 3.50 ( s, 1H), 3.28-3.22 (m, | |
| | 4H), 3.14-3.07 (m, 2H), 2.93-2.83 (m, 2H), 2.75-2.61 (m, 5H), 2.44 | |
| | (s, 3H), 2.03-1.95 (m, 1H), 1.90-1.80 (m, 3H), 1.79-1.71 (m, 1H), | |
| | 1.54-1.40 (m, 1H), 1.39-1.23 (m, 2H) | |
| 101 | ¹H NMR (400 MHz, DMSO-d₆) δ 11.05 (s, 1H), 9.93 (s, 1H), 8.79 (s, | A |
| | 1H), 8.66 (d, J = 8.4 Hz, 1H), 8.44 (dd, J = 6.0, 7.2 Hz, 1H), 8.16 (s, | |
| | 1H), 7.81 (s, 1H), 7.70 (d, J = 8.4 Hz, 1H), 7.52 (dd, J = 2.0, 10.4 Hz, | |
| | 1H), 7.46 (d, J = 8.8 Hz, 1H), 6.96 (dt, J = 2.0, 7.6 Hz, 1H), 6.89 (d, J = | |
| | 8.0 Hz, 1H), 6.84 (d, J = 8.8Hz, 1H), 6.27 (s, 1H), 6.08 ( d, J = 7.2 Hz, | |
| | 1H), 5.31-5.22 (m, 1H), 4.38 (s, 2H), 3.82-3.66 (m, 4H), 3.27 (s, 3H), | |
| | 2.94-2.87 (m, 1H), 2.82 ( s, 4H), 2.74-2.56 (m, 3H), 2.55-2.51 (m, | |
| | 3H), 2.45 (s, 3H), 2.42 (s, 3H), 2.31 ( d, J = 10.4 Hz, 2H), 2.03-1.84 | |
| | (m, 3H) | |
| 102 | ¹H NMR (400 MHz, DMSO-d₆) δ 11.05 (s, 1H), 9.98 (s, 1H), 8.81 (s, | A |
| | 1H), 8.72 (d, J = 8.8 Hz, 1H), 8.48-8.39 (m, 1H), 7.82 (s, 1H), 7.71 (d, | |
| | J = 8.4 Hz, 1H), 7.59-7.48 (m, 2H), 6.96 (dt, J = 2.8, 7.6 Hz, 1H), 6.89 | |
| | (d, J = 8.4 Hz, 1H), 6.85-6.78 (m, 1H), 6.27 (d, J = 2.0 Hz, 1H), 6.08 | |
| | (dd, J = 2.0, 8.4 Hz, 1H), 5.26 (dd, J = 5.2, 12.8 Hz, 1H), 4.45-4.32 (m, | |
| | 2H), 3.83-3.66 (m, 4H), 3.28 (s, 3H), 2.97 (d, J = 10.4 Hz, 2H), 2.93- | |
| | 2.81 (m, 1H), 2.73-2.60 (m, 3H), 2.59-2.52 (m, 2H), 2.43 ( s, 3H), | |
| | 2.38-2.24 (m, 3H), 2.17-2.05 (m, 2H), 2.01-1.85 (m, 3H), 1.74- | |
| | 1.54 (m, 4H) | |
| 103 | ¹H NMR (400 MHz, DMSO-d₆) δ 11.07 (s, 1H), 9.95 (s, 1H), 8.79 (s, | A |
| | 1H), 8.66 (d, J = 8.4 Hz, 1H), 8.48-8.43 (m, 1H), 7.82 (s, 1H), 7.71 (d, | |
| | J = 8.4 Hz, 1H), 7.66 (d, J = 8.4 Hz, 1H), 7.55-7.47 (m, 2H), 6.97 (dt, J = | |
| | 2.4, 7.2 Hz, 1H), 6.92 (d, J = 2.0 Hz, 1H), 6.87-6.81 (m, 2H), 4.39 | |
| | (s, 2H), 3.60 (dd, J = 7.6, 10.4 Hz, 1H), 3.54-3.50 (m, 1H), 3.46-3.38 | |
| | (m, 2H), 3.37 (d, J = 1.2 Hz, 1H), 3.19 (dd, J = 7.2, 9.6 Hz, 1H), 2.92- | |
| | 2.83 (m, 5H), 2.69-2.67 (m, 1H), 2.62-2.60 (m, 2H), 2.55 (d, J = 4.8 | |
| | Hz, 3H), 2.49-2.43 (m, 5H), 2.21-2.12 (m, 1H), 2.06-1.98 (m, 1H), | |
| | 1.80 (dd, J = 8.4, 12.8 Hz, 1H) | |
| 104 | ¹H NMR (400 MHz, DMSO-d₆) δ 11.07 (s, 1H), 9.98 (s, 1H), 8.81 (s, | A |
| | 1H), 8.73 (d, J = 8.4 Hz, 1H), 8.45 (dd, J = 6.0, 7.2 Hz, 1H), 8.18 (s, | |
| | 1H), 7.82 (s, 1H), 7.72 (d, J = 8.4 Hz, 1H), 7.65 (d, J = 8.4 Hz, 1H), | |
| | 7.58 (d, J = 8.4 Hz, 1H), 7.52 (dd, J = 2.4, 10.0 Hz, 1H), 6.96 (dt, J = | |
| | 2.8, 7.6 Hz, 1H), 6.91 (s, 1H), 6.82 (d, J = 8.4 Hz, 2H), 5.06 (dd, J = | |
| | 5.2, 12.8 Hz, 1H), 4.39 (s, 2H), 3.60-3.56 (m, 1H), 3.53-3.48 (m, | |
| | 2H), 3.17 (dd, J = 6.8, 10.0 Hz, 2H), 3.11-3.07 (m, 1H), 3.04-2.99 | |
| | (m, 1H), 2.91-2.83 (m, 1H), 2.68-2.59 (m, 3H), 2.58-2.53 (m, 3H), | |
| | 2.44-2.39 (m, 2H), 2.16-1.98 (m, 4H), 1.79 (s, 1H), 1.73-1.64 (m, | |
| | 4H) | |
| 105 | ¹H NMR (400 MHz, MeOD-d₄) δ 8.83 (d, J = 8.8 Hz, 1H), 8.41-8.24 | A |
| | (m, 2H), 7.75-7.66 (m, 2H), 7.58 (d, J = 8.0 Hz, 1H), 7.34 (dd, J = 2.4, | |
| | 9.3 Hz, 1H), 7.24 (t, J = 8.0 Hz, 1H), 7.01-6.91 (m, 1H), 6.87 (d, J = | |
| | 8.4 Hz, 1H), 6.79 (d, J = 8.4 Hz, 1H), 6.65 (d, J = 8.0 Hz, 1H), 5.29- | |
| | 5.11 (m, 1H), 4.76-4.53 (m, 2H), 4.39 (s, 2H), 3.79-3.68 (m, 2H), | |
| | 3.59 (s, 1H), 3.50-3.43 (m, 2H), 3.27-3.10 (m, 5H), 2.95-2.68 (m, | |
| | 5H), 2.62 (s, 3H), 2.16-1.91 (m, 8H), 1.66-1.47 (m, 2H) | |
| 106 | ¹H NMR (400 MHz, MeOD-d₄) δ 8.70 (d, J = 8.4 Hz, 1H), 8.36 (s, 1H), | A |
| | 8.32 (dd, J = 5.4, 7.6 Hz, 1H), 7.71-7.63 (m, 2H), 7.57-7.49 (m, 1H), | |
| | 7.33 (dd, J = 2.4, 9.2 Hz, 1H), 7.26-7.18 (m, 1H), 6.98-6.90 (m, 1H), | |
| | 6.88 (d, J = 8.4 Hz, 1H), 6.78 (d, J = 8.4 Hz, 1H), 6.63 (d, J = 7.6 Hz, | |
| | 1H), 5.23-5.16 (m, 1H), 4.60 (s, 1H), 4.37 (s, 2H), 3.58 (s, 2H), 3.50- | |
| | 3.37 (m, 3H), 3.13-3.03 (m, 6H), 2.94-2.63 (m, 8H), 2.54 (s, 3H), | |
| | 2.13-2.05 (m, 1H), 2.00-1.87 (m, 3H), 1.58-1.42 (m, 2H) | |
| 107 | ¹H NMR (400 MHz, DMSO-d₆) δ 11.05 (s, 1H), 9.95 (s, 1H), 8.80 (s, | A |
| | 1H), 8.66 (d, J = 8.6 Hz, 1H), 8.44 (dd, J = 6.2, 7.3 Hz, 1H), 8.13 (s, | |
| | 1H), 7.82 (s, 1H), 7.71 (d, J = 8.6 Hz, 1H), 7.56-7.43 (m, 2H), 7.15 (t, | |
| | J = 8.1 Hz, 1H), 6.96 (dt, J = 2.6, 7.5 Hz, 1H), 6.85 (d, J = 8.6 Hz, 1H), | |
| | 6.65 (d, J = 8.0 Hz, 1H), 6.60-6.50 (m, 1H), 5.28-5.14 (m, 1H), 4.38 | |
| | (s, 2H), 3.55 (br s, 2H), 3.43-3.36 (m, 3H), 2.98-2.79 (m, 6H), 2.70- | |
| | 2.54 (m, 8H), 2.47 (s, 3H), 1.99-1.86 (m, 1H), 1.83-1.74 (m, 2H), | |
| | 1.60-1.42 (m, 3H), 1.36-1.26 (m, 2H) | |
| 108 | ¹H NMR (400 MHz, DMSO-d₆) δ 11.08 (s, 1H), 9.92 (s, 1H), 8.78 (s, | C |
| | 1H), 8.65 (d, J = 8.4 Hz, 1H), 8.48-8.40 (m, 1H), 8.21 (s, 1H), 7.81 (s, | |
| | 1H), 7.70 (d, J = 8.8 Hz, 1H), 7.52 (dd, J = 2.4, 10.0 Hz, 1H), 7.44 (d, | |
| | J = 8.4 Hz, 1H), 7.11 (s, 1H), 7.02-6.90 (m, 3H), 6.84 (d, J = 8.4 Hz, | |
| | 1H), 5.38-5.29 (m, 1H), 4.43-4.32 (m, 2H), 3.33 (s, 3H), 3.05-2.95 | |
| | (m, 4H), 2.75-2.57 (m, 5H), 2.52-2.51 (m, 3H), 2.45 (s, 3H), 2.28- | |
| | 2.21 (m, 2H), 2.06-1.95 (m, 3H), 1.84 (dd, J = 1.6, 11.2 Hz, 2H), 1.75 | |
| | d, J = 4.4 Hz, 3H), 1.37-1.26 (m, 2H) | |
| 109 | ¹H NMR (400 MHz, DMSO-d₆) δ 10.98 (s, 1H), 10.19 (s, 1H), 8.89 (s, | A |
| | 1H), 8.53 (d, J = 8.4 Hz, 1H), 8.46 (dd, J = 6.0, 7.2 Hz, 1H), 8.13 (s, | |
| | 1H), 7.84 (s, 1H), 7.81-7.76 (m, 2H), 7.65 (d, J = 8.0 Hz, 1H), 7.53 | |
| | (dd, J = 2.4, 10.0 Hz, 1H), 7.27 (d, J = 8.4 Hz, 1H), 7.00-6.95 (m, 1H), | |
| | 6.95-6.91 (m, 1H), 5.09 (dd, J = 5.2, 13.2 Hz, 1H), 4.44-4.38 (m, | |
| | 3H), 4.25 (d, J = 17.2 Hz, 1H), 3.46-3.37 (m, 3H), 3.16-2.99 (m, 2H), | |
| | 2.98-2.81 (m, 2H), 2.81-2.70 (m, 3H), 2.04-1.94 (m, 2H), 1.91- | |
| | 1.82 (m, 3H), 1.82-1.66 (m, 6H), 1.43-1.26 (m, 3H) | |
| 110 | ¹H NMR (400 MHz, DMSO-d₆) δ 11.10 (s, 1H), 9.99 (s, 1H), 8.82 (s, | A |
| | 1H), 8.74 (d, J = 8.4 Hz, 1H), 8.51-8.39 (m, 1H), 8.23 (s, 1H), 7.83 (s, | |
| | 1H), 7.73 (d, J = 8.8 Hz, 1H), 7.61-7.48 (m, 2H), 7.03-6.80 (m, 5H), | |
| | 5.36 (dd, J = 5.2, 12.8 Hz, 1H), 4.39 (s, 2H), 3.64 (s, 3H), 3.14 (d, J = | |
| | 9.6 Hz, 3H), 2.99-2.80 (m, 4H), 2.75-2.65 (m, 4H), 2.60 (s, 1H), 2.46 | |
| | (s, 3H), 2.37-2.28 (m, 2H), 2.01 (dd, J = 4.8, 10.4 Hz, 1H), 1.89 (d, J = | |
| | 12.4 Hz, 2H), 1.66 (d, J = 9.6 Hz, 1H), 1.57-1.44 (m, 3H), 1.41-1.28 | |
| | (m, 2H) | |
| 111 | ¹H NMR (400 MHz, MeOD-d₄) δ 8.64 (d, J = 8.4 Hz, 1H), 8.30 (dd, J = | A |
| | 5.6, 7.5 Hz, 1H), 7.71-7.62 (m, 2H), 7.45 (d, J = 8.8 Hz, 1H), 7.32 (dd, | |
| | J = 2.4, 9.4 Hz, 1H), 7.10-6.99 (m, 2H), 6.96-6.83 (m, 3H), 5.32 (dd, | |
| | J = 5.2, 12.4 Hz, 1H), 4.58 (br s, 2H), 4.51-4.44 (m, 1H), 4.36 (s, 2H), | |
| | 4.30-4.23 (m, 1H), 3.76 (s, 3H), 3.70-3.61 (m, 1H), 3.50-3.45 (m, | |
| | 1H), 3.27-3.23 (m, 2H), 2.93-2.84 (m, 2H), 2.83-2.77 (m, 2H), 2.65 | |
| | (s, 4H), 2.54 (s, 3H), 2.37-2.27 (m, 2H), 2.20-2.10 (m, 1H), 1.98 (br | |
| | d, J = 10.6 Hz, 2H), 1.73-1.54 (m, 2H) | |
| 112 | ¹H NMR (400 MHz, CDCl₃) δ 10.99 (s, 1H), 10.09 (s, 1H), 8.90-8.73 | A |
| | (m, 2H), 8.63 (dd, J = 5.6, 6.8 Hz, 1H), 8.34-8.14 (m, 2H), 7.86-7.64 | |
| | (m, 3H), 7.50 (d, J = 8.8 Hz, 1H), 7.35-7.27 (m, 2H), 6.98-6.90 (m, | |
| | 1H), 5.17-5.04 (m, 1H), 4.48-4.36 (m, 3H), 4.33-4.21 (m, 1H), 3.61 | |
| | (br s, 1H), 2.91 (br t, J = 5.2 Hz, 1H), 2.85-2.72 (m, 4H), 2.62 (br d, J = | |
| | 2.4 Hz, 1H), 2.57 (s, 3H), 2.43 (br d, J = 2.4 Hz, 2H), 2.17-2.10 (m, | |
| | 1H), 2.08-1.94 (m, 5H), 1.93-1.79 (m, 4H), 1.56-1.51 (m, 1H), 1.46 | |
| | (br d, J = 3.2 Hz, 6H), 1.31-1.23 (m, 2H) | |
| 113 | ¹H NMR (400 MHz, DMSO-d₆) δ 8 11.00 (s, 1H), 9.94 (s, 1H), 8.80 (s, | B |
| | 1H), 8.66 (d, J = 8.8 Hz, 1H), 8.45 (dd, J = 6.0, 7.6 Hz, 1H), 7.82 (s, | |
| | 1H), 7.71 (d, J = 8.4 Hz, 1H), 7.65 (d, J = 8.0 Hz, 1H), 7.52 (dd, J = 2.4, | |
| | 10.0 Hz, 1H), 7.47 (d, J = 8.8 Hz, 1H), 7.29 (d, J = 8.4 Hz, 1H), 6.96 | |
| | (dt, J = 2.8, 7.6 Hz, 1H), 6.84 (d, J = 8.4 Hz, 1H), 5.10 (dd, J = 4.8, 13.2 | |
| | Hz, 1H), 4.47-4.34 (m, 3H), 4.26 (d, J = 17.6 Hz, 1H), 3.52-3.42 (m, | |
| | 2H), 2.97-2.69 (m, 11H), 2.64-2.53 (m, 2H), 2.47 (s, 3H), 2.45-2.39 | |
| | (m, 1H), 2.04-1.90 (m, 3H), 1.74-1.61 (m, 2H) | |
| 114 | ¹H NMR (400 MHz, DMSO-d₆) δ 10.99 (br s, 1H), 10.13 (s, 1H), 8.85 | A |
| | (s, 1H), 8.77 (d, J = 8.8 Hz, 1H), 8.51-8.40 (m, 1H), 8.18 (s, 1H), 7.83 | |
| | (s, 1H), 7.78-7.69 (m, 2H), 7.65 (d, J = 8.0 Hz, 1H), 7.52 (dd, J = 2.6, | |
| | 10.4 Hz, 1H), 7.28 (d, J = 8.0 Hz, 1H), 7.07-7.02 (m, 1H), 6.97 (dt, J = | |
| | 2.8, 7.2 Hz, 1H), 5.68-5.44 (m, 2H), 5.10 (dd, J = 4.9, 13.2 Hz, 1H), | |
| | 4.45-4.21 (m, 4H), 3.00 (br d, J = 11.2 Hz, 2H), 2.90 (br dd, J = 3.4, 14 | |
| | Hz, 2H), 2.82-2.72 (m, 3H), 2.59-2.55 (m, 1H), 2.41 (br s, 1H), 2.30- | |
| | 2.24 (m, 2H), 2.11-1.94 (m, 4H), 1.92-1.85 (m, 2H), 1.74-1.67 (m, | |
| | 4H), 1.36-1.23 (m, 3H) | |
| 115 | ¹H NMR (400 MHz, DMSO-d₆) δ 11.08 (s, 1H), 9.90 (s, 1H), 8.74 (s, | A |
| | 1H), 8.61 (d, J = 8.8 Hz, 1H), 7.53 (dd, J = 2.4, 8.4 Hz, 2H), 7.05 (s, | |
| | 1H), 7.01-6.94 (m, 1H), 6.88 (dd, J = 8.0, 14.0 Hz, 2H), 6.79 (d, J = | |
| | 8.4 Hz, 1H), 5.35 (dd, J = 5.2, 13.2 Hz, 1H), 4.47 (d, J = 18.0 Hz, 1H), | |
| | 4.27 (d, J = 18.4 Hz, 1H), 3.93-3.85 (m, 2H), 3.63 (s, 3H), 3.16-3.01 | |
| | (m, 5H), 2.98-2.84 (m, 3H), 2.77-2.58 (m, 6H), 2.42-2.30 (m, 3H), | |
| | 2.22-2.10 (m, 2H), 2.05-1.92 (m, 3H), 1.86 (d, J = 11.6 Hz, 2H), 1.76- | |
| | 1.63 (m, 5H), 1.60-1.48 (m, 1H), 1.42-1.29 (m, 2H), 1.09 (d, J = 6.4 | |
| | Hz, 3H) | |
| 116 | ¹H NMR (400 MHz, DMSO-d₆) δ 11.08 (s, 1H), 9.99 (s, 1H), 8.80 (s, | A |
| | 1H), 8.75-8.69 (m, 1H), 8.47-8.40 (m, 1H), 8.14 (s, 1H), 7.81 (s, 1H), | |
| | 7.71 (d, J = 8.4 Hz, 1H), 7.52 (dd, J = 2.4, 9.6 Hz, 2H), 6.99-6.88 (m, | |
| | 2H), 6.84 (d, J = 8.0 Hz, 1H), 6.55 (t, J = 6.8 Hz, 1H), 6.42 (t, J = 7.8 | |
| | Hz, 1H), 5.31 (dd, J = 4.8, 12.4 Hz, 1H), 4.38 (s, 2H), 3.83 (d, J = 5.6 | |
| | Hz, 2H), 3.70 (d, J = 8.4 Hz, 2H), 3.05 (s, 2H), 2.99 (s, 2H), 2.66-2.58 | |
| | (m, 6H), 2.54 (s, 3H), 2.08-1.93 (m, 5H), 1.78-1.59 (m, 8H) | |
| 117 | ¹H NMR (400 MHz, DMSO-d₆) δ 11.08 (s, 1H), 9.95 (s, 1H), 8.79 (s, | A |
| | 1H), 8.65 (d, J = 8.4 Hz, 1H), 8.47-8.39 (m, 1H), 7.81 (s, 1H), 7.70 (d, | |
| | J = 8.4 Hz, 1H), 7.51 (dd, J = 2.4, 10.0 Hz, 1H), 7.48-7.44 (m, 1H), | |
| | 6.99-6.88 (m, 2H), 6.85 (d, J = 8.8 Hz, 1H), 6.54 (t, J = 7.2 Hz, 1H), | |
| | 6.47-6.38 (m, 1H), 5.35-5.29 (m, 1H), 4.38 (s, 2H), 3.83 (d, J = 5.2 | |
| | Hz, 2H), 3.70 (d, J = 8.0 Hz, 2H), 3.05 (s, 2H), 2.99 (s, 2H), 2.90 (d, J = | |
| | 2.8 Hz, 8H), 2.67 (d, J = 2.0 Hz, 2H), 2.54 (s, 5H), 2.45 (s, 3H), 2.08- | |
| | 1.93 (m, 2H), 1.74-1.57 (m, 1H) | |
| 118 | ¹H NMR (400 MHz, DMSO-d₆) δ 10.99 (s, 1H), 10.25 (s, 1H), 8.91 (s, | A |
| | 1H), 8.58 (d, J = 8.4 Hz, 1H), 8.51-8.43 (m, 1H), 7.85 (s, 1H), 7.80 (d, | |
| | J = 8.4 Hz, 1H), 7.75-7.69 (m, 1H), 7.66 (d, J = 8.0 Hz, 1H), 7.53 (dd, | |
| | J = 2.4, 10.0 Hz, 1H), 7.28 (d, J = 8.0 Hz, 1H), 7.07 (d, J = 8.4 Hz, 1H), | |
| | 6.97 (dt, J = 2.4, 7.6 Hz, 1H), 5.10 (dd, J = 4.8, 13.2 Hz, 1H), 4.45- | |
| | 4.37 (m, 3H), 4.26 (d, J = 17.6 Hz, 1H), 3.49-3.36 (m, 3H), 2.97-2.86 | |
| | (m, 2H), 2.77 (t, J = 10.8 Hz, 2H), 2.65-2.52 (m, 3H), 2.47 (d, J = 5.2 | |
| | Hz, 1H), 2.45-2.38 (m, 1H), 2.04-1.94 (m, 2H), 1.94-1.65 (m, 7H), | |
| | 1.46-1.32 (m, 2H) | |
| 119 | ¹H NMR (400 MHz, DMSO-d₆) δ 10.93 (s, 1H), 9.99 (s, 1H), 8.80 (s, | A |
| | 1H), 8.73 (d, J = 8.4 Hz, 1H), 8.44 (dd, J = 5.9, 7.2 Hz, 1H), 8.13 (s, | |
| | 1H), 7.82 (s, 1H), 7.72 (d, J = 8.4 Hz, 1H), 7.56-7.43 (m, 3H), 6.96 (dt, | |
| | J = 2.8, 7.4 Hz, 1H), 6.84 (br d, J = 8.0 Hz, 1H), 6.54-6.43 (m, 2H), | |
| | 5.03 (dd, J = 5.0, 13.2 Hz, 1H), 4.39 (s, 2H), 4.34-4.27 (m, 1H), 4.23- | |
| | 4.13 (m, 1H), 3.64 (s, 2H), 3.59 (s, 2H), 3.37 (br d, J = 1.2 Hz, 1H), | |
| | 3.19-3.02 (m, 2H), 2.94-2.86 (m, 1H), 2.74 (br s, 1H), 2.61-2.54 (m, | |
| | 4H), 2.40-2.35 (m, 1H), 2.02-1.87 (m, 4H), 1.74 (br s, 8H), 1.60- | |
| | 1.46 (m, 3H), 1.09-0.91 (m, 2H) | |
| 120 | ¹H NMR (400 MHz, DMSO-d₆) δ 10.93 (s, 1H), 9.98 (s, 1H), 8.79 (s, | A |
| | 1H), 8.72 (d, J = 8.5 Hz, 1H), 8.47-8.39 (m, 1H), 8.15 (s, 1H), 7.81 (s, | |
| | 1H), 7.71 (d, J = 8.6 Hz, 1H), 7.56-7.45 (m, 3H), 7.08-7.00 (m, 2H), | |
| | 6.96 (dt, J = 2.5, 7.5 Hz, 1H), 6.83 (d, J = 8.6 Hz, 1H), 5.03 (dd, J = 5.0, | |
| | 13.3 Hz, 1H), 4.38 (s, 2H), 4.34-4.27 (m, 1H), 4.22-4.14 (m, 1H), | |
| | 3.20 (br d, J = 2.9 Hz, 4H), 3.13-3.02 (m, 3H), 2.94-2.83 (m, 2H), | |
| | 2.64-2.53 (m, 6H), 2.41-2.33 (m, 2H), 2.09-1.87 (m, 4H), 1.75- | |
| | 1.62 (m, 6H), 1.59-1.46 (m, 4H) | |
| 121 | ¹H NMR (400 MHz, DMSO-d₆) δ 10.95 (s, 1H), 10.08 (s, 1H), 9.44- | A |
| | 9.29 (m, 1H), 8.87 (s, 1H), 8.77 (d, J = 8.4 Hz, 1H), 8.68 (dd, J = 5.4, | |
| | 7.2 Hz, 1H), 8.27 (s, 1H), 7.94 (dd, J = 2.3, 8.8 Hz, 1H), 7.77 (d, J = 8.8 | |
| | Hz, 1H), 7.49 (d, J = 8.4 Hz, 1H), 7.40 (dt, J = 2.3, 7.6 Hz, 1H), 7.30 (d, | |
| | J = 8.0 Hz, 1H), 6.93 (d, J = 8.4 Hz, 1H), 6.51 (d, J = 8.4 Hz, 1H), 5.04 | |
| | (dd, J = 5.1, 13.2 Hz, 1H), 4.48-4.26 (m, 4H), 4.06 (s, 2H), 3.90 (s, | |
| | 2H), 3.83 (br d, J = 4.8 Hz, 1H), 3.74 (s, 3H), 3.19 (br d, J = 4.8 Hz, | |
| | 3H), 3.11 (br d, J = 11.2 Hz, 4H), 2.93-2.86 (m, 1H), 2.65-2.59 (m, | |
| | 1H), 2.56-2.53 (m, 3H), 2.43 (br d, J = 5.6 Hz, 2H), 2.13-2.03 (m, | |
| | 2H), 2.01-1.75 (m, 6H) | |
| 122 | ¹H NMR (400 MHz, DMSO-d₆) δ 10.94 (s, 1H), 9.94 (s, 1H), 8.78 (s, | A |
| | 1H), 8.65 (d, J = 8.4 Hz, 1H), 8.49-8.36 (m, 1H), 8.13 (s, 1H), 7.81 (s, | |
| | 1H), 7.70 (d, J = 8.5 Hz, 1H), 7.57-7.40 (m, 2H), 7.28 (d, J = 8.1 Hz, | |
| | 1H), 6.96 (dt, J = 2.7, 7.5 Hz, 1H), 6.84 (d, J = 8.5 Hz, 1H), 6.56-6.42 | |
| | (m, 1H), 5.03 (dd, J = 5.1, 13.4 Hz, 1H), 4.50-4.23 (m, 4H), 4.02 (s, | |
| | 2H), 3.88 (s, 2H), 3.79-3.61 (m, 4H), 3.26-3.20 (m, 2H), 2.93-2.81 | |
| | (m, 5H), 2.64-2.56 (m, 5H), 2.47-2.35 (m, 6H), 2.04-1.82 (m, 3H) | |
| 123 | ¹H NMR (400 MHz, DMSO-d₆) δ 10.97 (s, 1H), 9.97 (s, 1H), 8.79 (s, | A |
| | 1H), 8.73 (d, J = 8.8 Hz, 1H), 8.47-8.39 (m, 1H), 7.81 (s, 1H), 7.71 (d, | |
| | J = 8.4 Hz, 1H), 7.56-7.48 (m, 2H), 7.41 (d, J = 8.4 Hz, 1H), 7.26 (dd, | |
| | J = 2.0, 8.4 Hz, 1H), 7.16 (d, J = 2.4 Hz, 1H), 6.97 (dt, J = 2.8, 7.6 Hz, | |
| | 1H), 6.84 (d, J = 8.4 Hz, 1H), 5.08 (dd, J = 5.2, 13.2 Hz, 1H), 4.38 (s, | |
| | 2H), 4.33 (d, J = 16.4 Hz, 1H), 4.23-4.17 (m, 1H), 3.75 (d, J = 12.4 | |
| | Hz, 2H), 2.95-2.85 (m, 2H), 2.77-2.66 (m, 4H), 2.63-2.56 (m, 3H), | |
| | 2.42-2.32 (m, 4H), 1.99 (td, J = 5.2, 10.4 Hz, 1H), 1.84-1.63 (m, 7H), | |
| | 1.51 (s, 3H), 1.36-1.21 (m, 3H) | |
| 124 | ¹H NMR (400 MHz, DMSO-d₆) δ 11.08 (s, 1H), 10.03-9.90 (m, 1H), | A |
| | 8.80 (s, 1H), 8.72 (d, J = 8.4 Hz, 1H), 8.44 (dd, J = 6.0, 7.2 Hz, 1H), | |
| | 7.82 (s, 1H), 7.71 (d, J = 8.4 Hz, 1H), 7.59-7.47 (m, 2H), 7.00-6.90 | |
| | (m, 2H), 6.82 (d, J = 8.4 Hz, 1H), 6.71 (d, J = 8.0 Hz, 1H), 6.65 (d, J = | |
| | 8.0 Hz, 1H), 5.36-5.26 (m, 1H), 4.38 (s, 2H), 3.58 ( s, 1H), 3.57 (s, | |
| | 3H), 3.54-3.51 (m, 2H), 3.02-2.81 (m, 4H), 2.70-2.60 (m, 3H), 2.51 | |
| | ( s, 3H), 2.15 ( d, J = 6.8 Hz, 2H), 2.05-1.92 (m, 5H), 1.77-1.60 (m, | |
| | 6H), 1.55-1.42 (m, 3H), 0.97 ( d, J = 12.0 Hz, 2H) | |
| 125 | ¹H NMR (400 MHz, DMSO-d₆) δ 11.08 (s, 1H), 9.99 (s, 1H), 8.80 (s, | A |
| | 1H), 8.64 (d, J = 8.4 Hz, 1H), 8.40 (dd, J = 6.2, 7.2 Hz, 1H), 7.81 (s, | |
| | 1H), 7.66 (t, J = 9.2 Hz, 2H), 7.51 (dd, J = 2.6, 10.0 Hz, 1H), 7.04-6.82 | |
| | (m, 5H), 5.34 (br dd, J = 5.2, 12.8 Hz, 1H), 4.45-4.29 (m, 2H), 3.98 (s, | |
| | 2H), 3.63 (s, 3H), 3.18-2.94 (m, 6H), 2.94-2.77 (m, 3H), 2.75-2.63 | |
| | (m, 4H), 2.29-2.19 (m, 2H), 2.09-1.95 (m, 3H), 1.91-1.79 (m, 2H), | |
| | 1.76-1.60 (m, 5H), 1.41-1.28 (m, 2H) | |
| 126 | ¹H NMR (400 MHz, DMSO-d₆) δ 10.96 (s, 1H), 9.84 (s, 1H), 8.76 (s, | A |
| | 1H), 8.60 (d, J = 8.8 Hz, 1H), 8.44 (dd, J = 6.0, 7.2 Hz, 1H), 8.16 (s, | |
| | 1H), 7.80 (s, 1H), 7.68 (d, J = 8.8 Hz, 1H), 7.52 (dd, J = 2.8, 10.0 Hz, | |
| | 1H), 7.36 (d, J = 8.0 Hz, 1H), 7.32 (d, J = 8.8 Hz, 1H), 6.96 (dt, J = 2.8, | |
| | 7.6 Hz, 1H), 6.80 (d, J = 8.4 Hz, 1H), 6.60 (t, J = 8.0 Hz, 1H), 5.04 (dd, | |
| | J = 5.2, 13.2 Hz, 1H), 4.48-4.32 (m, 3H), 4.24 (d, J = 16.8 Hz, 1H), | |
| | 4.16-4.04 (m, 3H), 3.96 (s, 2H), 3.40-3.36 (m, 4H), 3.12-3.06 (m, | |
| | 2H), 2.96-2.84 (m, 2H), 2.56 (d, J = 15.6 Hz, 1H), 2.44 (s, 3H), 2.40- | |
| | 2.32 (m, 4H), 2.08-1.88 (m, 5H) | |
| 127 | ¹H NMR (400 MHz, DMSO-d₆) δ 8 10.96 (s, 1H), 10.03 (s, 1H), 8.81 (s, | A |
| | 1H), 8.74 (d, J = 8.4 Hz, 1H), 8.42 (dd, J = 6.0, 7.2 Hz, 1H), 7.82 (s, | |
| | 1H), 7.71 (d, J = 8.4 Hz, 1H), 7.63 (d, J = 8.0 Hz, 1H), 7.52 (dd, J = 2.4, | |
| | 10.0 Hz, 1H), 7.38 (d, J = 8.0 Hz, 1H), 7.02-6.88 (m, 2H), 6.63-6.52 | |
| | (m, 1H), 5.18 (t, J = 5.2 Hz, 1H), 5.03 (dd, J = 5.0, 13.2 Hz, 1H), 4.63 | |
| | (d, J = 4.8 Hz, 2H), 4.47-4.36 (m, 3H), 4.26 (d, J = 16.4 Hz, 1H), 4.09 | |
| | (s, 2H), 3.96 (s, 2H), 3.11 (d, J = 8.4 Hz, 2H), 2.97-2.84 (m, 3H), | |
| | 2.62-2.53 (m, 4H), 2.43-2.34 (m, 4H), 2.01-1.92 (m, 3H), 1.75 (s, 4H) | |
| 128 | ¹H NMR (400 MHz, DMSO-d₆) δ 10.81 (s, 1H), 10.03 (s, 1H), 8.84- | A |
| | 8.80 (m, 1H), 8.79-8.72 (m, 1H), 8.53-8.47 (m, 1H), 7.96-7.92 (m, | |
| | 2H), 7.73 (d, J = 8.4 Hz, 1H), 7.64 (dd, J = 2.4, 9.6 Hz, 1H), 7.46 (dd, | |
| | J = 8.4, 12.8 Hz, 2H), 7.13-7.06 (m, 1H), 6.90 (d, J = 8.4 Hz, 2H), 4.39 | |
| | (s, 2H), 4.29-4.24 (m, 2H), 3.78-3.73 (m, 4H), 3.08 (s, 1H), 2.84 (t, J = | |
| | 12.4 Hz, 3H), 2.68-2.63 (m, 2H), 2.54 (s, 3H), 2.19 (dd, J = 4.4, 12.8 | |
| | Hz, 1H), 2.02-1.94 (m, 2H), 1.90-1.83 (m, 2H), 1.81-1.76 (m, 2H), | |
| | 1.68-1.57 (m, 4H), 1.54 (d, J = 7.2 Hz, 1H), 1.41 (d, J = 6.8 Hz, 2H), | |
| | 1.28-1.13 (m, 3H) | |
| 129 | ¹H NMR (400 MHz, DMSO-d₆) δ 11.02-10.91 (m, 1H), 9.78 (s, 1H), | A |
| | 8.75 (s, 1H), 8.56 (d, J = 8.4 Hz, 1H), 8.42 (dd, J = 6.0, 7.4 Hz, 1H), | |
| | 8.28 (s, 1H), 7.80 (s, 1H), 7.67 (d, J = 8.4 Hz, 1H), 7.51 (dd, J = 2.4, | |
| | 10.0 Hz, 1H), 7.35 (d, J = 8.0 Hz, 1H), 7.21 (d, J = 8.8 Hz, 1H), 6.96 | |
| | (dt, J = 2.4, 7.6 Hz, 1H), 6.78 (d, J = 8.4 Hz, 1H), 6.57 (t, J = 8.0 Hz, | |
| | 1H), 5.03 (dd, J = 4.8, 13.2 Hz, 1H), 4.45-4.34 (m, 3H), 4.28-4.20 | |
| | (m, 1H), 4.05 (s, 2H), 3.92 (s, 3H), 3.22 (d, J = 2.0 Hz, 3H), 2.98-2.79 | |
| | (m, 3H), 2.67-2.54 (m, 3H), 2.43 (s, 3H), 2.40-2.35 (m, 1H), 2.30- | |
| | 2.21 (m, 3H), 1.98-1.84 (m, 3H), 1.80-1.70 (m, 2H) | |
| 130 | ¹H NMR (400 MHz, DMSO-d₆) δ 10.99-10.89 (m, 1H), 9.89 (s, 1H), | A |
| | 8.74 (s, 1H), 8.61 (d, J = 8.4 Hz, 1H), 8.16 (s, 1H), 7.52 (d, J = 8.8 Hz, | |
| | 2H), 7.37 (d, J = 8.0 Hz, 1H), 7.04 (s, 1H), 6.78 (d, J = 8.4 Hz, 1H), | |
| | 6.64-6.49 (m, 1H), 5.04 (dd, J = 4.8, 13.2 Hz, 1H), 4.51-4.39 (m, | |
| | 2H), 4.30-4.21 (m, 2H), 4.08 (s, 2H), 3.97-3.83 (m, 4H), 3.01-2.85 | |
| | (m, 4H), 2.68-2.53 (m, 3H), 2.49-2.48 (m, 3H), 2.45-2.31 (m, 7H), | |
| | 2.15-2.06 (m, 2H), 1.99-1.87 (m, 4H), 1.72-1.49 (m, 5H), 1.09 (d, | |
| | J = 6.4 Hz, 3H) | |
| 131 | ¹H NMR (400 MHz, DMSO-d₆) δ 11.05 (s, 1H), 9.90 (s, 1H), 8.74 (s, | A |
| | 1H), 8.61 (d, J = 8.8 Hz, 1H), 8.17 (s, 1H), 7.59-7.45 (m, 2H), 7.04 (s, | |
| | 1H), 6.93 (d, J = 8.4 Hz, 1H), 6.85-6.73 (m, 2H), 6.64 (dd, J = 2.0, 8.8 | |
| | Hz, 1H), 5.35-5.18 (m, 1H), 4.47 ( d, J = 18.0 Hz, 1H), 4.27 ( d, J = | |
| | 18.8 Hz, 1H), 3.93-3.85 (m, 2H), 3.59 ( d, J = 11.6 Hz, 2H), 3.31 ( s, | |
| | 3H), 3.03-2.85 (m, 4H), 2.70-2.58 (m, 5H), 2.51-2.51 (m, 3H), 2.39- | |
| | 2.33 (m, 1H), 2.24 ( d, J = 6.8 Hz, 2H), 2.10-1.92 (m, 5H), 1.83 ( d, | |
| | J = 12.0 Hz, 2H), 1.73-1.60 (m, 5H), 1.58-1.49 (m, 1H), 1.34-1.22 (m, | |
| | 2H), 1.09 ( d, J = 6.4 Hz, 3H) | |
| 132 | ¹H NMR (400 MHz, DMSO-d₆) δ 10.96 (s, 1H), 9.92 (s, 1H), 8.80 (s, | A |
| | 1H), 8.76 (d, J = 8.8 Hz, 1H), 8.44 (dd, J = 6.0, 7.6 Hz, 1H), 8.16 (s, | |
| | 1H), 7.80 (s, 1H), 7.72 (d, J = 8.4 Hz, 1H), 7.56 (dd, J = 2.8, 10.0 Hz, | |
| | 1H), 7.40-7.32 (m, 1H), 6.96 (dt, J = 2.8, 7.6 Hz, 1H), 6.68 (s, 1H), | |
| | 6.60 (t, J = 8.0 Hz, 1H), 5.02 (dd, J = 5.2, 13.2 Hz, 1H), 4.48-4.36 (m, | |
| | 3H), 4.28 (d, J = 16.8 Hz, 1H), 4.16-4.04 (m, 2H), 4.00-3.92 (m, 2H), | |
| | 3.20 (d, J = 8.8 Hz, 3H), 3.06-2.84 (m, 3H), 2.80 (s, 2H), 2.60-2.52 | |
| | (m, 6H), 2.44-2.40 (m, 2H), 2.36 (s, 3H), 2.24-2.16 (m, 2H), 2.04- | |
| | 1.96 (m, 2H), 1.96-1.92 (m, 1H), 1.68 (d, J = 12.4 Hz, 2H) | |
| 133 | ¹H NMR (400 MHz, DMSO-d₆) δ 11.08 (s, 1H), 9.97 (s, 1H), 8.82 (s, | B |
| | 1H), 8.68 (d, J = 8.4 Hz, 1H), 8.47-8.41 (m, 1H), 8.13 (s, 1H), 7.82 (s, | |
| | 1H), 7.71 (d, J = 8.4 Hz, 1H), 7.55-7.46 (m, 2H), 7.01-6.92 (m, 2H), | |
| | 6.90-6.82 (m, 2H), 6.67 (dd, J = 2.0, 8.4 Hz, 1H), 5.35-5.26 (m, 1H), | |
| | 4.39 (s, 2H), 3.79-3.67 (m, 2H), 3.06-2.85 (m, 7H), 2.74-2.58 (m, | |
| | 5H), 2.48 (s, 7H), 2.44 (s, 1H), 2.06-1.94 (m, 3H), 1.75-1.63 (m, 2H) | |
| 134 | ¹H NMR (400 MHz, DMSO-d₆) δ 11.09 (s, 1H), 9.96 (s, 1H), 8.82- | B |
| | 8.76 (m, 1H), 8.67 (d, J = 8.4 Hz, 1H), 8.44 (dd, J = 6.0, 7.4 Hz, 1H), | |
| | 7.82 (s, 1H), 7.71 (d, J = 8.8 Hz, 1H), 7.57-7.42 (m, 2H), 7.02-6.83 | |
| | (m, 5H), 5.41-5.29 (m, 1H), 4.38 (s, 2H), 3.65 (s, 3H), 3.25-3.14 (m, | |
| | 3H), 2.90 ( d, J = 3.6 Hz, 6H), 2.82-2.57 (m, 7H), 2.48 (s, 3H), 2.08- | |
| | 1.90 (m, 3H), 1.84-1.63 (m, 2H) | |
| 135 | ¹H NMR (400 MHz, DMSO-d₆) δ 11.10 (s, 1H), 10.08 (s, 1H), 8.88 (s, | A |
| | 1H), 8.84-8.76 (m, 1H), 8.72 (d, J = 8.4 Hz, 1H), 8.36 (dd, J = 6.0, 7.2 | |
| | Hz, 1H), 7.88 (s, 1H), 7.68-7.60 (m, 2H), 7.48 (d, J = 8.4 Hz, 1H), | |
| | 7.06 (dt, J = 2.4, 7.6 Hz, 1H), 7.02-6.96 (m, 1H), 6.96-6.88 (m, 3H), | |
| | 5.36 (dd, J = 5.6, 12.8 Hz, 1H), 4.80 (q, J = 6.4 Hz, 1H), 3.64 (s, 3H), | |
| | 3.24-3.08 (m, 7H), 3.06-3.00 (m, 1H), 2.92-2.84 (m, 1H), 2.76- | |
| | 2.72 (m, 2H), 2.68-2.64 (m, 1H), 2.56-2.52 (m, 4H), 2.10-1.84 (m, | |
| | 8H), 1.56-1.40 (m, 2H), 0.80 (d, J = 6.8 Hz, 3H) | |
| 136 | ¹H NMR (400 MHz, DMSO-d₆) δ 10.95 (s, 1H), 10.00 (s, 1H), 8.80 (s, | A |
| | 1H), 8.77-8.69 (m, 1H), 8.43 (t, J = 6.4 Hz, 1H), 7.81 (s, 1H), 7.72 (d, | |
| | J = 8.4 Hz, 1H), 7.51 (dd, J = 2.0, 9.6 Hz, 2H), 7.38 (d, J = 8.0 Hz, 1H), | |
| | 6.97 (dt, J = 2.8, 7.2 Hz, 1H), 6.86 (d, J = 5.2 Hz, 1H), 6.63-6.57 (m, | |
| | 1H), 5.06-5.00 (m, 1H), 4.48-4.35 (m, 4H), 4.26 (d, J = 16.4 Hz, 2H), | |
| | 4.11 (s, 2H), 3.96 (d, J = 1.2 Hz, 2H), 2.61 (d, J = 2.4 Hz, 2H), 2.52 (s, | |
| | 5H), 2.45-2.36 (m, 4H), 2.07-1.93 (m, 4H), 1.85-1.67 (m, 3H), | |
| | 1.33-1.18 (m, 5H) | |
| 137 | ¹H NMR (400 MHz, DMSO-d₆) δ 10.81 (s, 1H), 10.00 (s, 1H), 8.81 (s, | A |
| | 1H), 8.68 (d, J = 8.4 Hz, 1H), 8.48-8.39 (m, 1H), 8.13 (s, 1H), 7.94 (d, | |
| | J = 2.4 Hz, 1H), 7.82 (s, 1H), 7.71 (d, J = 8.4 Hz, 1H), 7.57-7.47 (m, | |
| | 2H), 7.38 (dd, J = 2.4, 8.8 Hz, 1H), 6.97 (dt, J = 2.6, 7.6 Hz, 1H), 6.88 | |
| | (d, J = 8.8 Hz, 1H), 6.82 (d, J = 8.8 Hz, 1H), 4.39 (s, 2H), 4.28 (br d, J = | |
| | 11.8 Hz, 2H), 3.73 (dd, J = 4.9, 12.4 Hz, 1H), 3.54-3.45 (m, 1H), | |
| | 3.26-2.88 (m, 8H), 2.84-2.70 (m, 3H), 2.69-2.63 (m, 1H), 2.56-2.51 (m, | |
| | 2H), 2.21-2.12 (m, 1H), 2.02-1.92 (m, 1H), 1.77 (br d, J = 12.0 Hz, | |
| | 2H), 1.71-1.56 (m, 3H), 1.36 (br d, J = 2.0 Hz, 3H), 1.29-1.10 (m, | |
| | 3H | |
| 138 | ¹H NMR (400 MHz, DMSO-d₆) δ 10.96 (s, 1H), 10.09 (s, 1H), 8.85 (s, | A |
| | 1H), 8.83-8.76 (m, 1H), 8.67 (dd, J = 5.6, 7.6 Hz, 1H), 7.99 (d, J = 8.4 | |
| | Hz, 1H), 7.75 (dd, J = 2.0, 9.2 Hz, 1H), 7.56 ( d, J = 8.4 Hz, 1H), 7.37 | |
| | (d, J = 8.0 Hz, 1H), 7.07 (dt, J = 2.4, 7.6 Hz, 1H), 6.89-6.81 (m, 1H), | |
| | 6.59 (t, J = 8.0 Hz, 1H), 5.09-4.98 (m, 1H), 4.54 (s, 2H), 4.43 (d, J = | |
| | 17.2 Hz, 1H), 4.25 (d, J = 17.2 Hz, 1H), 4.09 (s, 2H), 3.99-3.91 (m, | |
| | 2H), 3.13-3.02 (m, 3H), 2.98-2.84 (m, 2H), 2.60 ( d, J = 2.8 Hz, 2H), | |
| | 2.53 ( s, 3H), 2.37-2.31 (m, 3H), 2.03-1.91 (m, 4H), 1.78-1.64 (m, | |
| | 4H), 1.27-1.21 (m, 2H) | |
| 139 | ¹H NMR (400 MHz, DMSO-d₆) δ 11.08 (s, 1H), 10.08 (s, 1H), 8.86- | A |
| | 8.76 (m, 2H), 8.68 (dd, J = 5.6, 7.6 Hz, 1H), 7.99 (d, J = 8.8 Hz, 1H), | |
| | 7.71 (s, 1H), 7.58 (d, J = 8.4 Hz, 1H), 7.10-7.03 (m, 1H), 7.01-6.95 | |
| | (m, 1H), 6.93-6.81 (m, 3H), 5.39-5.30 (m, 1H), 4.54 (s, 2H), 3.64 (s, | |
| | 3H), 3.16-3.08 (m, 3H), 3.01 ( d, J = 10.4 Hz, 2H), 2.75-2.62 (m, | |
| | 5H), 2.53 (s, 3H), 2.31-2.24 (m, 2H), 2.12-1.97 (m, 3H), 1.85 ( d, J = | |
| | 11.6 Hz, 2H), 1.75-1.62 (m, 5H), 1.42-1.28 (m, 2H) | |
| 140 | ¹H NMR (400 MHz, DMSO-d₆) δ 11.05 (s, 1H), 10.08 (s, 1H), 8.87- | A |
| | 8.77 (m, 2H), 8.68 (dd, J = 5.6, 7.6 Hz, 1H), 8.15 (s, 1H), 7.99 (d, J = | |
| | 8.8 Hz, 1H), 7.75 (dd, J = 2.4, 9.6 Hz, 1H), 7.58 (d, J = 8.4 Hz, 1H), | |
| | 7.07 (dt, J = 2.4, 7.6 Hz, 1H), 6.93 (d, J = 8.4 Hz, 1H), 6.88-6.78 (m, | |
| | 2H), 6.64 (dd, J = 2.0, 8.8 Hz, 1H), 5.28 (dd, J = 5.2, 13.2 Hz, 1H), | |
| | 4.61-4.46 (m, 2H), 3.59 ( d, J = 12.0 Hz, 3H), 3.31 ( s, 3H), 3.00 ( d, J = | |
| | 10.4 Hz, 2H), 2.93-2.84 (m, 1H), 2.70-2.62 (m, 4H), 2.53 (s, 3H), | |
| | 2.25 ( d, J = 6.8 Hz, 2H), 2.11-1.95 (m, 3H), 1.83 ( d, J = 11.6 Hz, | |
| | 2H), 1.75-1.62 (m, 5H), 1.34-1.22 (m, 2H) | |
| 141 | ¹H NMR (400 MHz, DMSO-d₆) δ 10.96 (s, 1H), 9.94 (s, 1H), 8.80 (s, | A |
| | 1H), 8.72 (d, J = 8.8 Hz, 1H), 8.47-8.39 (m, 1H), 7.81 (s, 1H), 7.70 (d, | |
| | J = 8.4 Hz, 1H), 7.52 (dd, J = 2.4, 10.0 Hz, 1H), 7.38 (d, J = 8.0 Hz, | |
| | 1H), 6.96 (dt, J = 2.4, 7.6 Hz, 1H), 6.68 (s, 1H), 6.59 (t, J = 8.0 Hz, 1H), | |
| | 5.04 (dd, J = 5.2, 13.2 Hz, 1H), 4.47-4.33 (m, 3H), 4.26 (d, J = 16.8 | |
| | Hz, 1H), 4.09 (s, 2H), 3.96 (s, 2H), 3.06 (d, J = 3.2 Hz, 4H), 2.95-2.84 | |
| | (m, 2H), 2.72-2.65 (m, 2H), 2.58 (dd, J = 2.0, 16.0 Hz, 3H), 2.47 (s, | |
| | 5H), 2.42-2.33 (m, 3H), 2.29 (s, 3H), 2.01-1.92 (m, 3H) | |
| 142 | ¹H NMR (400 MHz, DMSO-d₆) δ 10.95 (s, 1H), 10.30 (d, J = 2.0 Hz, | A |
| | 1H), 8.89-8.81 (m, 2H), 8.47 (dd, J = 6.0, 7.2 Hz, 1H), 7.84 (s, 1H), | |
| | 7.75 (dd, J = 5.2, 8.8 Hz, 2H), 7.59-7.49 (m, 2H), 6.96 (m, J = 2.8, 7.6 | |
| | Hz, 1H), 6.91 (d, J = 8.8 Hz, 1H), 5.07 (dd, J = 4.8, 13.2 Hz, 1H), 4.51- | |
| | 4.37 (m, 4H), 4.26 (d, J = 17.2 Hz, 1H), 4.09 (d, J = 17.2 Hz, 1H), | |
| | 3.03-2.81 (m, 8H), 2.65-2.53 (m, 4H), 2.47-2.40 (m, 4H), 2.36 (dd, J = | |
| | 4.4, 12.8 Hz, 1H), 2.33-2.28 (m, 1H), 2.00-1.89 (m, 2H), 1.82 (d, J = | |
| | 12.0 Hz, 2H), 1.12 (d, J = 11.2 Hz, 2H) | |
| 143 | ¹H NMR (400 MHz, DMSO-d₆) δ 11.09 (s, 1H), 10.36 (d, J = 2.4 Hz, | A |
| | 1H), 8.94-8.84 (m, 2H), 8.50-8.43 (m, 1H), 7.84 (s, 1H), 7.77 (d, J = | |
| | 8.4 Hz, 1H), 7.60-7.50 (m, 2H), 7.00-6.94 (m, 2H), 6.88 (dd, J = 7.6, | |
| | 14.4 Hz, 2H), 5.35 (dd, J = 5.2, 12.8 Hz, 1H), 4.42 (s, 2H), 3.63 (s, 3H), | |
| | 3.13 (d, J = 10.4 Hz, 2H), 2.99 (d, J = 9.6 Hz, 2H), 2.74-2.58 (m, 7H), | |
| | 2.25 (d, J = 3.6 Hz, 2H), 2.08-1.95 (m, 4H), 1.88-1.82 (m, 2H), 1.75- | |
| | 1.59 (m, 6H), 1.33 (d, J = 12.0 Hz, 2H) | |
| 144 | ¹H NMR (400 MHz, DMSO-d₆) δ 10.96 (s, 1H), 9.96 (s, 1H), 8.80 (s, | A |
| | 1H), 8.60 (d, J = 8.8 Hz, 1H), 8.48-8.36 (m, 1H), 7.80 (s, 1H), 7.72 (d, | |
| | J = 8.8 Hz, 1H), 7.68 (d, J = 8.4 Hz, 1H), 7.64 (d, J = 8.0 Hz, 1H), 7.52 | |
| | (dd, J = 2.8, 10.0 Hz, 1H), 7.28 (d, J = 8.4 Hz, 1H), 6.96 (dt, J = 2.8, 7.6 | |
| | Hz, 1H), 6.88 (d, J = 8.4 Hz, 1H), 5.28 (s, 1H), 5.16-5.02 (m, 1H), | |
| | 4.44-4.36 (m, 3H), 4.28 (d, J = 17.6 Hz, 1H), 3.76-3.68 (m, 1H), 2.96 | |
| | (d, J = 9.6 Hz, 2H), 2.88 (td, J = 4.8, 12.8 Hz, 1H), 2.76 (t, J = 11.2 Hz, | |
| | 2H), 2.56 (s, 4H), 2.28 (d, J = 7.2 Hz, 2H), 2.04-1.96 (m, 3H), 1.88 (d, | |
| | J = 11.2 Hz, 2H), 1.68 (d, J = 0.8 Hz, 5H), 1.60 (s, 6H), 1.40-1.28 (m, | |
| | 2H) | |
| 145 | ¹H NMR (400 MHz, DMSO-d₆) δ 11.09 (s, 1H), 10.04 (s, 1H), 8.82 (s, | A |
| | 1H), 8.68 (d, J = 8.4 Hz, 1H), 8.43 (dd, J = 6.0, 7.2 Hz, 1H), 8.21 (d, J = | |
| | 2.0 Hz, 1H), 8.14 (s, 1H), 7.82 (s, 1H), 7.72 (d, J = 8.8 Hz, 1H), 7.62 | |
| | (dd, J = 2.4, 8.4 Hz, 1H), 7.52 (dd, J = 2.4, 10.0 Hz, 1H), 7.00-6.94 (m, | |
| | 3H), 6.88 (dd, J = 8.0, 12.4 Hz, 2H), 5.39-5.30 (m, 1H), 4.39 (s, 2H), | |
| | 3.64 (s, 3H), 3.12 (s, 4H), 2.95-2.83 (m, 2H), 2.74-2.66 (m, 3H), 2.65- | |
| | 2.55 (m, 3H), 2.31-2.15 (m, 2H), 2.02-1.96 (m, 1H), 1.88-1.70 (m, | |
| | 7H), 1.44-1.28 (m, 2H) | |
| 146 | ¹H NMR (400 MHz, DMSO-d₆) δ 10.95 (s, 1H), 10.05 (s, 1H), 8.82 (s, | A |
| | 1H), 8.68 (d, J = 8.4 Hz, 1H), 8.42 (t, J = 6.8 Hz, 1H), 8.20 (d, J = 1.6 | |
| | Hz, 1H), 7.82 (s, 1H), 7.72 (d, J = 8.8 Hz, 1H), 7.65-7.58 (m, 1H), | |
| | 7.52 (dd, J = 2.4, 10.0 Hz, 1H), 7.38 (d, J = 8.0 Hz, 1H), 7.02-6.94 (m, | |
| | 2H), 6.59 (t, J = 8.0 Hz, 1H), 5.04 (dd, J = 5.2, 13.2 Hz, 1H), 4.47-4.36 | |
| | (m, 3H), 4.26 (d, J = 16.8 Hz, 1H), 4.10 (s, 2H), 3.96 (s, 2H), 3.16 (s, | |
| | 3H), 2.99-2.83 (m, 2H), 2.69-2.53 (m, 4H), 2.43-2.30 (m, 4H), 1.97 | |
| | (d, J = 8.0 Hz, 3H), 1.90-1.68 (m, 4H) | |
| 147 | ¹H NMR (400 MHz, DMSO-d₆) δ 11.08 (s, 1H), 9.96 (s, 1H), 8.80 (s, | A |
| | 1H), 8.68 (d, J = 8.4 Hz, 1H), 8.52-8.40 (m, 1H), 7.80 (s, 1H), 7.72 (d, | |
| | J = 8.4 Hz, 1H), 7.64 (d, J = 8.4 Hz, 1H), 7.52 (dd, J = 2.8, 10.0 Hz, | |
| | 1H), 7.48 (d, J = 8.8 Hz, 1H), 6.96 (dt, J = 2.8, 7.6 Hz, 1H), 6.84 (d, J = | |
| | 8.8 Hz, 1H), 6.76 (d, J = 1.6 Hz, 1H), 6.64 (dd, J = 2.0, 8.4 Hz, 1H), | |
| | 5.06 (dd, J = 5.6, 12.8 Hz, 1H), 4.40 (s, 2H), 4.16 (t, J = 8.0 Hz, 2H), | |
| | 3.76-3.68 (m, 2H), 2.92-2.76 (m, 6H), 2.56 (d, J = 6.8 Hz, 6H), 2.48 | |
| | (s, 3H), 2.36 (t, J = 6.8 Hz, 2H), 2.04-1.96 (m, 1H), 1.88-1.76 (m, | |
| | 2H) | |
| 148 | ¹H NMR (400 MHz, DMSO-d₆) δ 11.07 (s, 1H), 10.06 (s, 1H), 8.83 (s, | A |
| | 1H), 8.72-8.65 (m, 1H), 8.46-8.38 (m, 1H), 8.21 (s, 1H), 7.82 (s, 1H), | |
| | 7.73 (d, J = 8.4 Hz, 1H), 7.62 (d, J = 8.8 Hz, 1H), 7.52 (dd, J = 2.4, 10.0 | |
| | Hz, 1H), 7.02-6.91 (m, 3H), 6.83 (s, 1H), 6.64 (d, J = 8.4 Hz, 1H), | |
| | 5.29 (dd, J = 5.2, 12.8 Hz, 1H), 4.39 (s, 2H), 3.62 (d, J = 11.6 Hz, 4H), | |
| | 3.31 (s, 3H), 2.94-2.85 (m, 2H), 2.70-2.58 (m, 8H), 2.01-1.96 (m, | |
| | 1H), 1.92-1.79 (m, 7H), 1.40-1.28 (m, 2H) | |
| 149 | ¹H NMR (400 MHz, DMSO-d₆) δ 10.94 (s, 1H), 9.86 (s, 1H), 8.73 (s, | A |
| | 1H), 8.54 (d, J = 8.4 Hz, 1H), 8.14 (s, 1H), 7.74 (d, J = 8.8 Hz, 1H), | |
| | 7.51 (d, J = 8.4 Hz, 1H), 7.45 (d, J = 8.4 Hz, 1H), 7.04 (s, 1H), 6.90 (d, | |
| | J = 9.2 Hz, 1H), 6.80 (d, J = 8.8 Hz, 1H), 5.06 (dd, J = 5.2, 13.2 Hz, | |
| | 1H), 4.53-4.40 (m, 3H), 4.26 (d, J = 17.6 Hz, 2H), 4.09 (d, J = 17.2 | |
| | Hz, 1H), 3.93-3.85 (m, 2H), 3.01-2.88 (m, 5H), 2.83 ( s, 4H), 2.63- | |
| | 2.51 (m, 4H), 2.44 (s, 3H), 2.42-2.30 (m, 3H), 2.23 ( d, J = 7.2 Hz, | |
| | 2H), 2.06-1.87 (m, 4H), 1.82 ( d, J = 13.2 Hz, 2H), 1.61-1.45 (m, | |
| | 1H), 1.17-1.10 (m, 1H), 1.08 (d, J = 6.4 Hz, 3H) | |
| 150 | ¹H NMR (400 MHz, DMSO-d₆) δ 11.09 (s, 1H), 10.03 (s, 1H), 8.82 (s, | B |
| | 1H), 8.74 (d, J = 8.4 Hz, 1H), 8.44 (dd, J = 6.0, 7.6 Hz, 1H), 7.82 (s, | |
| | 1H), 7.72 (d, J = 8.4 Hz, 1H), 7.52 (dd, J = 2.8, 10.0 Hz, 2H), 7.04- | |
| | 6.86 (m, 5H), 5.36 ( dd, J = 5.6, 12.8 Hz, 1H), 4.39 (s, 2H), 3.65 (s, 3H), | |
| | 3.53-3.45 (m, 2H), 3.24 ( s, 3H), 3.08-2.92 (m, 3H), 2.91-2.76 (m, | |
| | 3H), 2.73-2.60 (m, 2H), 2.55 (s, 3H), 2.16-2.05 (m, 2H), 2.03-1.96 | |
| | (m, 1H), 1.96-1.76 (m, 6H) | |
| 151 | ¹H NMR (400 MHz, DMSO-d₆) δ 10.96 (s, 1H), 8.88 (s, 1H), 8.77 (s, | A |
| | 1H), 8.53 (dd, J = 6.0, 7.2 Hz, 1H), 7.90 (s, 1H), 7.80-7.70 (m, 2H), | |
| | 7.56 (dd, J = 2.4, 10.0 Hz, 1H), 7.44 (d, J = 8.8 Hz, 1H), 7.02 (dt, J = | |
| | 2.8, 7.6 Hz, 1H), 6.92 (d, J = 8.8 Hz, 1H), 6.78 (8dd, J = 2.8, 8.4 Hz, | |
| | 1H), 5.07 (dd, J = 5.2, 13.2 Hz, 1H), 4.45 (d, J = 10.8 Hz, 2H), 4.38 (s, | |
| | 2H), 4.26 (d, J = 17.2 Hz, 1H), 4.09 (d, J = 17.2 Hz, 1H), 3.31-3.21 | |
| | (m, 2H), 3.08-2.77 (m, 8H), 2.58 (d, J = 17.6 Hz, 2H), 2.44-2.25 (m, | |
| | 5H), 2.06-1.91 (m, 2H), 1.83 (d, J = 11.6 Hz, 2H), 1.25-1.04 (m, 2H) | |
| 152 | ¹H NMR (400 MHz, DMSO-d₆) δ 11.02-10.92 (m, 1H), 9.96 (s, 1H), | A |
| | 8.88-8.76 (m, 1H), 8.72-8.60 (m, 1H), 8.44 (dd, J = 6.0, 7.6 Hz, 1H), | |
| | 7.84-7.80 (m, 1H), 7.72 (d, J = 8.4 Hz, 1H), 7.52 (dd, J = 2.8, 9.6 Hz, | |
| | 1H), 7.48 (d, J = 8.8 Hz, 1H), 7.36 (d, J = 8.0 Hz, 1H), 6.96 (dt, J = 2.8, | |
| | 7.6 Hz, 1H), 6.88-6.80 (m, 1H), 6.60 (t, J = 8.0 Hz, 1H), 5.02 (dd, J = | |
| | 4.8, 13.2 Hz, 1H), 4.44 (d, J = 16.8 Hz, 1H), 4.40 (s, 2H), 4.28 (d, J = | |
| | 16.4 Hz, 1H), 4.16 (t, J = 7.2 Hz, 2H), 3.76-3.68 (m, 2H), 3.40-3.36 | |
| | (m, 2H), 3.32-3.28 (m, 2H), 2.84 (s, 4H), 2.60 (d, J = 2.0 Hz, 1H), 2.56 | |
| | (s, 2H), 2.44 (s, 3H), 2.40-2.32 (m, 3H), 2.00-1.92 (m, 1H), 1.88- | |
| | 1.76 (m, 2H) | |
| 153 | ¹H NMR (400 MHz, DMSO-d₆) δ 10.99 (s, 1H), 10.30 (d, J = 2.4 Hz, | B |
| | 1H), 8.93-8.79 (m, 2H), 8.47 (dd, J = 6.0, 7.2 Hz, 1H), 7.84 (s, 1H), | |
| | 7.76 (d, J = 8.4 Hz, 1H), 7.65 (d, J = 8.0 Hz, 1H), 7.58-7.49 (m, 2H), | |
| | 7.29 (d, J = 8.4 Hz, 1H), 6.97 (dt, J = 2.8, 7.6 Hz, 1H), 5.10 (dd, J = 5.2, | |
| | 13.2 Hz, 1H), 4.47-4.35 (m, 3H), 4.26 (d, J = 17.6 Hz, 1H), 3.50-3.44 | |
| | (m, 2H), 2.93-2.83 (m, 5H), 2.82-2.74 (m, 2H), 2.74-2.65 (m, 4H), | |
| | 2.61-2.55 (m, 1H), 2.48-2.42 (m, 5H), 2.02-1.89 (m, 3H), 1.73- | |
| | 1.61 (m, 2H) | |
| 154 | ¹H NMR (400 MHz, DMSO-d₆) δ 10.96 (s, 1H), 9.93 (s, 1H), 8.80 (s, | A |
| | 1H), 8.65 (d, J = 8.4 Hz, 1H), 8.44 (dd, J = 6.0, 7.2 Hz, 1H), 7.81 (s, | |
| | 1H), 7.71 (dd, J = 8.8, 12.8 Hz, 2H), 7.51 (dd, J = 2.8, 10.0 Hz, 1H), | |
| | 7.44 (d, J = 8.8 Hz, 1H), 6.96 (dt, J = 2.8, 7.2 Hz, 1H), 6.83 (d, J = 8.4 | |
| | Hz, 1H), 6.34 (d, J = 8.4 Hz, 1H), 5.06 (dd, J = 5.2, 13.2 Hz, 1H), 4.38 | |
| | (s, 2H), 4.24 (d, J = 17.6 Hz, 1H), 4.11-4.05 (m, 3H), 3.95 (s, 2H), | |
| | 2.96-2.76 (m, 6H), 2.63-2.53 (m, 3H), 2.46-2.37 (m, 7H), 2.36- | |
| | 2.29 (m, 3H), 1.99-1.88 (m, 3H) | |
| 155 | ¹H NMR (400 MHz, DMSO-d₆) δ 10.97 (s, 1H), 9.98 (d, J = 0.8 Hz, | A |
| | 1H), 8.82 (s, 1H), 8.73 (d, J = 8.4 Hz, 1H), 8.45 (t, J = 6.8 Hz, 1H), 7.82 | |
| | (s, 1H), 7.72 (d, J = 8.4 Hz, 1H), 7.53 (dd, J = 2.4, 10.0 Hz, 2H), 7.37 | |
| | (d, J = 8.0 Hz, 1H), 7.02-6.92 (m, 1H), 6.82 (s, 1H), 6.64-6.54 (m, | |
| | 1H), 5.04 (dd, J = 5.2, 13.2 Hz, 1H), 4.46-4.34 (m, 3H), 4.25 (d, J = | |
| | 16.8 Hz, 1H), 4.07 (s, 2H), 3.97 (s, 2H), 2.96-2.86 (m, 3H), 2.59 (d, J = | |
| | 5.6 Hz, 2H), 2.54 (s, 3H), 2.42-2.36 (m, 2H), 2.33 (s, 2H), 1.99- | |
| | 1.88 (m, 3H), 1.73-1.58 (m, 3H), 1.56-1.39 (m, 3H), 1.27-1.10 (m, | |
| | 6H) | |
| 156 | ¹H NMR (400 MHz, DMSO-d₆) δ 10.67 (s, 1H), 10.06 (s, 1H), 8.85 (s, | A |
| | 1H), 8.75 (d, J = 8.4 Hz, 1H), 8.54-8.44 (m, 1H), 7.94-7.85 (m, 2H), | |
| | 7.74 (d, J = 8.4 Hz, 1H), 7.61 (dd, J = 2.4, 9.6 Hz, 1H), 7.54-7.44 (m, | |
| | 2H), 7.05 (d, J = 2.4 Hz, 1H), 6.95-6.81 (m, 2H), 6.60 (d, J = 6.8 Hz, | |
| | 1H), 4.40 (s, 2H), 4.35-4.24 (m, 2H), 3.77 (s, 2H), 3.70-3.62 (m, 2H), | |
| | 3.12 (s, 4H), 3.04 (s, 1H), 2.87-2.75 (m, 4H), 2.56 (s, 3H), 2.54 (s, | |
| | 1H), 2.09 (s, 1H), 2.00-1.89 (m, 5H), 1.55-1.41 (m, 2H) | |
| 157 | ¹H NMR (400 MHz, DMSO-d₆) δ 9.02 (d, J = 8.4 Hz, 1H), 8.38 (s, 1H), | B |
| | 8.34-8.28 (m, 1H), 7.72-7.68 (m, 3H), 7.50 (d, J = 12.4 Hz, 1H), 7.34- | |
| | 7.30 (m, 2H), 6.97-6.88 (m, 2H), 4.46 (d, J = 11.6 Hz, 2H), 4.39 (s, | |
| | 2H), 3.71 (t, J = 12.4 Hz, 6H), 3.26-3.19 (m, 2H), 2.96-2.87 (m, 3H), | |
| | 2.82-2.76 (m, 1H), 2.66 (s, 1H), 2.59 (s, 3H), 2.54-2.46 (m, 1H), 2.33 | |
| | (d, J = 12.0 Hz, 2H), 2.21-2.02 (m, 8H) | |
| 158 | ¹H NMR (400 MHz, DMSO-d₆) δ 10.976 (s, 1H), 10.00-9.92 (m, 1H), | B |
| | 8.80 (s, 1H), 8.68 (d, J = 8.4 Hz, 1H), 8.48-8.40 (m, 1H), 7.80 (s, 1H), | |
| | 7.72 (d, J = 8.8 Hz, 1H), 7.56-7.52 (m, 1H), 7.48 (d, J = 8.8 Hz, 1H), | |
| | 7.40 (d, J = 8.0 Hz, 1H), 7.12-7.06 (m, 1H), 6.96 (dt, J = 2.8, 7.6 Hz, | |
| | 1H), 6.84 (d, J = 8.8 Hz, 1H), 5.06 (dd, J = 5.2, 13.6 Hz, 1H), 4.44 | |
| | (d, J = 17.2 Hz, 1H), 4.36 (s, 2H), 4.32-4.24 (m, 1H), 3.88 (s, 3H), 3.72- | |
| | 3.56 (m, 2H), 3.06-2.84 (m, 6H), 2.84-2.76 (m, 2H), 2.72-2.64 (m, | |
| | 3H), 2.60-2.52 (m, 2H), 2.48 (s, 3H), 2.44-2.40 (m, 1H), 2.08-1.92 | |
| | (m, 3H), 1.76-1.60 (m, 2H) | |
| 159 | ¹H NMR (400 MHz, DMSO-d₆) δ 10.98-10.91 (m, 1H), 9.99 (s, 1H), | B |
| | 8.81 (s, 1H), 8.67 (d, J = 8.4 Hz, 1H), 8.43 (t, J = 6.4 Hz, 1H), 7.84- | |
| | 7.78 (m, 2H), 7.71 (d, J = 8.4 Hz, 1H), 7.55-7.46 (m, 2H), 7.04-6.94 | |
| | (m, 2H), 6.88 (d, J = 8.4 Hz, 1H), 5.08 (dd, J = 5.2, 13.2 Hz, 1H), 4.70- | |
| | 4.54 (m, 2H), 4.38 (s, 2H), 4.29 (d, J = 17.2 Hz, 1H), 4.12 (d, J = 17.2 | |
| | Hz, 1H), 3.60-3.52 (m, 2H), 3.10 (s, 2H), 3.04-2.86 (m, 5H), 2.68- | |
| | 2.53 (m, 4H), 2.48 (s, 3H), 2.41-2.29 (m, 2H), 1.98 (dd, J = 4.8, 10.5 | |
| | Hz, 2H), 1.73-1.55 (m, 2H) | |
| 160 | ¹H NMR (400 MHz, DMSO-d₆) δ 11.10 (s, 1H), 9.87 (s, 1H), 8.78 (s, | A |
| | 1H), 8.57 (d, J = 8.8 Hz, 1H), 8.41 (dd, J = 6.0, 7.2 Hz, 1H), 8.13 (s, | |
| | 1H), 8.02 (s, 1H), 7.81 (s, 1H), 7.69 (d, J = 8.8 Hz, 1H), 7.56-7.44 (m, | |
| | 2H), 7.01-6.94 (m, 3H), 6.89 (dd, J = 8.0, 12.0 Hz, 2H), 5.35 (dd, J = | |
| | 5.6, 12.0 Hz, 1H), 4.38 (s, 2H), 3.63 (s, 3H), 3.25-3.02 (m, 8H), 2.93- | |
| | 2.81 (m, 2H), 2.81-2.65 (m, 5H), 2.65-2.56 (m, 3H), 2.02-1.95 (m, | |
| | 1H), 1.86 (d, J = 12.0 Hz, 2H), 1.46-1.32 (m, 2H) | |
| 161 | ¹H NMR (400 MHz, DMSO-d₆) δ 11.00 (s, 1H), 9.91 (s, 1H), 8.85- | A |
| | 8.76 (m, 1H), 8.59 (d, J = 8.8 Hz, 1H), 8.41 (dd, J = 6.0, 7.2 Hz, 1H), | |
| | 8.11-8.00 (m, 1H), 7.82 (s, 1H), 7.74-7.62 (m, 2H), 7.59-7.44 (m, | |
| | 2H), 7.34-7.23 (m, 1H), 7.07-6.91 (m, 2H), 5.10 (dd, J = 4.8, 13.2 | |
| | Hz, 1H), 4.45-4.35 (m, 3H), 4.26 (d, J = 17.6 Hz, 1H), 3.93-3.58 (m, | |
| | 3H), 3.13 (dt, J = 2.4, 3.6 Hz, 3H), 3.00-2.85 (m, 2H), 2.84-2.73 (m, | |
| | 3H), 2.69-2.56 (m, 3H), 2.54 (s, 2H), 2.45-2.38 (m, 1H), 2.05-1.83 | |
| | (m, 4H), 1.53-1.34 (m, 2H) | |
| 162 | ¹H NMR (400 MHz, DMSO-d₆) δ 10.96 (s, 1H), 9.96-9.89 (m, 1H), | A |
| | 9.39-9.25 (m, 1H), 8.81 (s, 1H), 8.60 (d, J = 8.4 Hz, 1H), 8.43 (t, J = | |
| | 6.4 Hz, 1H), 8.07 (s, 1H), 7.86 (s, 1H), 7.78 (d, J = 8.8 Hz, 1H), 7.70 (d, | |
| | J = 8.4 Hz, 1H), 7.61-7.49 (m, 2H), 7.07-7.00 (m, 2H), 6.99-6.92 | |
| | (m, 1H), 5.08 (dd, J = 4.8, 12.8 Hz, 1H), 4.53-4.42 (m, 2H), 4.38 (s, | |
| | 2H), 4.26 (d, J = 17.6 Hz, 1H), 4.13-4.06 (m, 1H), 3.83-3.71 (m, 2H), | |
| | 3.68-3.58 (m, 2H), 3.12 (s, 2H), 3.00 (d, J = 2.0 Hz, 1H), 2.95-2.84 | |
| | (m, 2H), 2.59 (d, J = 17.6 Hz, 2H), 2.54 (d, J = 1.2 Hz, 2H), 2.36-2.31 | |
| | (m, 1H), 2.26-2.16 (m, 1H), 1.99-1.94 (m, 1H), 1.88-1.80 (m, 2H), | |
| | 1.27-1.17 (m, 2H) | |
| 163 | ¹H NMR (400 MHz, DMSO-d₆) δ 11.12 (s, 1H), 9.98 (s, 1H), 8.83 (s, | A |
| | 1H), 8.68 (d, J = 8.4 Hz, 1H), 8.45 (dd, J = 6.0, 7.2 Hz, 1H), 7.83 (s, | |
| | 1H), 7.72 (d, J = 8.4 Hz, 1H), 7.59-7.43 (m, 2H), 6.98 (dt, J = 2.4, 7.6 | |
| | Hz, 1H), 6.88 (d, J = 8.4 Hz, 2H), 6.82-6.75 (m, 1H), 5.34 (dd, J = 4.4, | |
| | 13.2 Hz, 1H), 4.83-4.72 (m, 1H), 4.40 (s, 2H), 3.25 (d, J = 10.4 Hz, | |
| | 3H), 3.17 (dd, J = 4.0, 6.8 Hz, 1H), 3.08-2.84 (m, 6H), 2.80-2.75 (m, | |
| | 1H), 2.74-2.63 (m, 5H), 2.59 (d, J = 10.4 Hz, 1H), 2.49 (s, 3H), 2.05- | |
| | 1.99 (m, 1H), 1.87 (d, J = 10.4 Hz, 2H), 1.48-1.35 (m, 9H) | |
| 164 | ¹H NMR (400 MHz, DMSO-d₆) δ 11.07 (s, 1H), 10.01 (s, 1H), 8.82 (s, | B |
| | 1H), 8.74 (d, J = 8.4 Hz, 1H), 8.45 (dd, J = 6.0, 7.2 Hz, 1H), 7.83 (s, | |
| | 1H), 7.73 (d, J = 8.4 Hz, 1H), 7.53 (dd, J = 2.4, 10.0 Hz, 2H), 7.01- | |
| | 6.93 (m, 2H), 6.87 (s, 2H), 6.67 (d, J = 8.0 Hz, 1H), 5.30 (d, J = 12.8 | |
| | Hz, 1H), 4.39 (s, 2H), 3.72 (s, 2H), 3.05 (s, 3H), 2.96-2.83 (m, 2H), | |
| | 2.75-2.58 (m, 6H), 2.55-2.53 (m, 3H), 2.33 (s, 3H), 2.10-1.93 (m, | |
| | 3H), 1.92-1.54 (m, 6H) | |
| 165 | ¹H NMR (400 MHz, DMSO-d₆) δ 11.00 (s, 1H), 9.99 (s, 1H), 8.82 (s, | B |
| | 1H), 8.73 (d, J = 8.4 Hz, 1H), 8.45 (dd, J = 6.0, 7.2 Hz, 1H), 8.19 (s, | |
| | 1H), 7.82 (s, 1H), 7.72 (d, J = 8.4 Hz, 1H), 7.64 (d, J = 8.0 Hz, 1H), | |
| | 7.56-7.48 (m, 2H), 7.30 (d, J = 8.4 Hz, 1H), 6.97 (dt, J = 2.8, 7.6 Hz, | |
| | 1H), 6.83 (d, J = 8.4 Hz, 1H), 5.10 (dd, J = 5.2, 13.2 Hz, 1H), 4.45- | |
| | 4.36 (m, 3H), 4.25 (d, J = 17.6 Hz, 1H), 3.12 (d, J = 10.0 Hz, 3H), 3.04 | |
| | (d, J = 0.8 Hz, 3H), 2.97-2.84 (m, 2H), 2.78-2.64 (m, 2H), 2.63-2.52 | |
| | (m, 4H), 2.48-2.38 (m, 2H), 2.02-1.95 (m, 1H), 1.84 (d, J = 12.0 Hz, | |
| | 2H), 1.78-1.64 (m, 8H), 1.57-1.46 (m, 4H), 1.22-1.12 (m, 2H) | |
| 166 | ¹H NMR (400 MHz, DMSO-d₆) δ 10.90 (d, J = 12.8 Hz, 1H), 9.95 (s, | B |
| | 1H), 8.81 (s, 1H), 8.66 (d, J = 8.4 Hz, 1H), 8.48-8.40 (m, 1H), 7.82 (s, | |
| | 1H), 7.74-7.68 (m, 2H), 7.52 (dd, J = 2.4, 10.1 Hz, 1H), 7.46 (d, J = | |
| | 8.4 Hz, 1H), 7.00-6.90 (m, 2H), 6.84 (d, J = 8.4 Hz, 1H), 4.77-4.66 | |
| | (m, 1H), 4.62-4.41 (m, 3H), 4.38 (s, 2H), 3.04-2.70 (m, 12H), 2.59 (t, | |
| | J = 9.2 Hz, 2H), 2.46 (s, 3H), 2.05-1.90 (m, 3H), 1.56-1.44 (m, 2H), | |
| | 1.40 (dd, J = 6.8, 14.2 Hz, 3H) | |
| 167 | ¹H NMR (400 MHz, DMSO-d₆) δ 10.97 (s, 1H), 9.84 (s, 1H), 8.77 (s, | A |
| | 1H), 8.56 (d, J = 8.4 Hz, 1H), 8.44-8.37 (m, 1H), 7.98 (d, J = 3.2 Hz, | |
| | 1H), 7.81 (s, 1H), 7.68 (d, J = 8.4 Hz, 1H), 7.52 (dd, J = 2.4, 10.0 Hz, | |
| | 1H), 7.43 (dd, J = 2.8, 9.2 Hz, 1H), 7.37 (d, J = 8.0 Hz, 1H), 7.01-6.93 | |
| | (m, 2H), 6.58 (t, J = 7.6 Hz, 1H), 5.09-4.99 (m, 1H), 4.43 (d, J = 16.8 | |
| | Hz, 1H), 4.39-4.33 (m, 2H), 4.25 (d, J = 16.8 Hz, 1H), 4.08 (s, 1H), | |
| | 3.95 (s, 1H), 3.36 (s, 2H), 3.32-3.30 (m, 2H), 3.09 (s, 4H), 2.94-2.87 | |
| | (m, 1H), 2.69-2.54 (m, 3H), 2.41-2.35 (m, 4H), 2.34-2.30 (m, 2H), | |
| | 1.98-1.87 (m, 3H) | |
| 168 | ¹H NMR (400 MHz, DMSO-d₆) δ 11.00 (s, 1H), 10.00 (s, 1H), 8.84 (s, | B |
| | 1H), 8.72 (d, J = 8.4 Hz, 1H), 8.44 (dd, J = 6.0, 7.2 Hz, 1H), 7.84 (s, | |
| | 1H), 7.72 (d, J = 8.8 Hz, 1H), 7.64 (d, J = 8.0 Hz, 1H), 7.56-7.48 (m, | |
| | 2H), 7.28 (d, J = 8.0 Hz, 1H), 6.96 (dt, J = 2.8, 7.56 Hz, 1H), 6.88 (d, J = | |
| | 8.4 Hz, 1H), 5.12 (dd, J = 4.8, 13.0 Hz, 1H), 4.44-4.36 (m, 3H), 4.24 | |
| | (d, J = 17.6 Hz, 1H), 3.28-3.12 (m, 3H), 3.06-2.84 (m, 6H), 2.60- | |
| | 2.56 (m, 1H), 2.52-2.48 (m, 3H), 2.48-2.36 (m, 2H), 2.26-1.92 (m, | |
| | 4H), 1.92-1.56 (m, 10H) | |
| 169 | ¹H NMR (400 MHz, DMSO-d₆) δ 11.00 (s, 1H), 9.92 (s, 1H), 8.80 (s, | B |
| | 1H), 8.68 (d, J = 8.4 Hz, 1H), 8.44 (dd, J = 6.0, 7.6 Hz, 1H), 7.80 (s, | |
| | 1H), 7.72 (d, J = 8.8 Hz, 1H), 7.64 (d, J = 8.0 Hz, 1H), 7.52 (dd, J = 2.8, | |
| | 10.0 Hz, 1H), 7.48 (d, J = 8.8 Hz, 1H), 7.28 (d, J = 8.4 Hz, 1H), 6.96 | |
| | (dt, J = 2.8, 7.6 Hz, 1H), 6.84 (d, J = 8.4 Hz, 1H), 5.12 (dd, J = 4.8, 13.2 | |
| | Hz, 1H), 4.44-4.36 (m, 3H), 4.24 (d, J = 17.2Hz, 1H), 3.02 (s, 2H), | |
| | 3.00-2.88 (m, 3H), 2.84 (s, 5H), 2.60-2.52 (m, 3H), 2.48-2.36 (m, | |
| | 6H), 2.08-1.96 (m, 3H), 1.76 (s, 2H), 1.72-1.60 (m, 4H) | |
| 170 | ¹H NMR (400 MHz, DMSO-d₆) δ 11.00 (s, 1H), 9.95 (s, 1H), 8.81 (s, | B |
| | 1H), 8.66 (d, J = 8.8 Hz, 1H), 8.44 (dd, J = 6.0, 7.2 Hz, 1H), 7.82 (s, | |
| | 1H), 7.71 (d, J = 8.4 Hz, 1H), 7.64 (d, J = 8.0 Hz, 1H), 7.52 (dd, J = 2.4, | |
| | 10.0 Hz, 1H), 7.46 (d, J = 8.4 Hz, 1H), 7.30 (d, J = 8.4 Hz, 1H), 6.96 | |
| | (m, J = 2.4, 7.2 Hz, 1H), 6.85 (d, J = 8.4 Hz, 1H), 5.10 (dd, J = 5.2, 13.2 | |
| | Hz, 1H), 4.44-4.35 (m, 3H), 4.25 (d, J = 17.2 Hz, 1H), 3.11-3.00 (m, | |
| | 5H), 2.89 (s, 8H), 2.63-2.54 (m, 2H), 2.46 (s, 3H), 2.44-2.38 (m, 1H), | |
| | 1.99 (d, J = 5.6 Hz, 1H), 1.84 (d, J = 12.0 Hz, 2H), 1.78-1.64 (m, 4H), | |
| | 1.50 (d, J = 6.4 Hz, 4H), 1.23-1.12 (m, 2H) | |
| 171 | ¹H NMR (400 MHz, DMSO-d₆) δ 10.96 (s, 1H), 10.16-10.04 (m, 1H), | A |
| | 8.84 (s, 1H), 8.72 (d, J = 8.8 Hz, 1H), 8.68 (dd, J = 5.6, 7.56 Hz, 1H), | |
| | 7.96 (d, J = 8.8 Hz, 1H), 7.76 (dd, J = 2.4, 9.6 Hz, 2H), 7.56-7.48 (m, | |
| | 1H), 7.06 (dt, J = 2.4, 7.42 Hz, 1H), 6.96-6.84 (m, 2H), 5.06 (dd, J = | |
| | 4.8, 13.12 Hz, 1H), 4.56 (s, 2H), 4.48 (dd, J = 1.6, 7.6 Hz, 2H), 4.28 (d, | |
| | J = 17.6 Hz, 1H), 4.102 (d, J = 17.2 Hz, 1H), 3.72-3.52 (m, 1H), 3.28- | |
| | 3.06 (m, 4H), 3.06-2.72 (m, 6H), 2.68-2.52 (m, 3H), 2.48-2.12 (m, | |
| | 4H), 1.88 (s, 4H), 1.28-1.12 (m, 2H) | |
| 172 | ¹H NMR (400 MHz, DMSO-d₆) δ 11.11 (s, 1H), 9.98-9.89 (m, 1H), | B |
| | 8.81 (s, 1H), 8.67 (d, J = 8.4 Hz, 1H), 8.49-8.41 (m, 1H), 7.82 (s, 1H), | |
| | 7.71 (d, J = 8.8 Hz, 1H), 7.57-7.42 (m, 2H), 7.00-6.93 (m, 1H), 6.90- | |
| | 6.76 (m, 3H), 5.38-5.29 (m, 1H), 4.84-4.72 (m, 1H), 4.39 (s, 2H), | |
| | 3.32-3.25 (m, 3H), 2.86 ( s, 4H), 2.74-2.64 (m, 6H), 2.61-2.52 (m, | |
| | 1H), 2.47 ( s, 3H), 2.45-2.35 (m, 2H), 2.04-1.97 (m, 1H), 1.96-1.87 | |
| | (m, 2H), 1.74-1.59 (m, 2H), 1.42 ( d, J = 6.8 Hz, 6H) | |
| 173 | ¹H NMR (400 MHz, DMSO-d₆) δ 11.08 (s, 1H), 9.94 (s, 1H), 8.81 (s, | B |
| | 1H), 8.67 (d, J = 8.8 Hz, 1H), 8.48-8.41 (m, 1H), 7.82 (s, 1H), 7.71 (d, | |
| | J = 8.8 Hz, 1H), 7.55-7.44 (m, 2H), 7.00-6.93 (m, 2H), 6.93-6.89 | |
| | (m, 1H), 6.85 (d, J = 8.4 Hz, 2H), 5.67-5.56 (m, 1H), 5.30 (dd, J = 5.2, | |
| | 12.0 Hz, 1H), 4.39 (s, 2H), 3.15 (d, J = 11.2 Hz, 2H), 2.86 (s, 5H), 2.71 | |
| | (s, 6H), 2.63 (s, 2H), 2.47 (s, 3H), 2.38 (s, 1H), 2.03-1.93 (m, 3H), | |
| | 1.63-1.54 (m, 2H), 1.49 (dd, J = 6.8, 10.4 Hz, 6H) | |
| 174 | ¹H NMR (400 MHz, DMSO-d₆) δ 11.08 (s, 1H), 10.02-9.86 (m, 1H), | A |
| | 8.80 (s, 1H), 8.68 (d, J = 8.0 Hz, 1H), 8.52-8.40 (m, 1H), 7.82 (s, 1H), | |
| | 7.72 (d, J = 8.68Hz, 1H), 7.68-7.60 (m, 1H), 7.56-7.52 (m, 1H), 7.52- | |
| | 7.44 (m, 1H), 6.96 (dt, J = 2.8, 7.6 Hz, 1H), 6.84 (d, J = 7.6 Hz, 1H), | |
| | 6.80 (s, 1H), 6.68 (d, J = 8.0 Hz, 1H), 5.12-5.02 (m, 1H), 4.40 (s, 2H), | |
| | 4.24-4.08 (m, 2H), 3.84-3.68 (m, 2H), 3.12-2.96 (m, 2H), 2.92- | |
| | 2.80 (m, 4H), 2.56 (s, 4H), 2.52 (s, 3H), 2.48 (s, 4H), 2.04-1.96 (m, | |
| | 1H) | |
| 175 | ¹H NMR (400 MHz, DMSO-d₆) δ 10.61 (s, 1H), 9.99 (s, 1H), 8.81 (s, | A |
| | 1H), 8.73 (d, J = 8.4 Hz, 1H), 8.44 (dd, J = 6.0, 7.2 Hz, 1H), 8.06 (d, J = | |
| | 7.6 Hz, 1H), 7.82 (s, 1H), 7.72 (d, J = 8.8 Hz, 1H), 7.59-7.48 (m, 2H), | |
| | 7.30 (s, 1H), 6.96 (dt, J = 2.8, 7.6 Hz, 1H), 6.90 (d, J = 7.2 Hz, 1H), | |
| | 6.84 (d, J = 8.4 Hz, 1H), 6.68 (d, J = 1.6 Hz, 1H), 4.38 (s, 2H), 3.83 (d, | |
| | J = 13.2 Hz, 2H), 3.75 (t, J = 6.8 Hz, 2H), 3.14-3.09 (m, 2H), 2.84- | |
| | 2.72 (m, 5H), 2.53-2.51 (m, 3H), 2.44-2.35 (m, 2H), 2.34-2.21 (m, | |
| | 2H), 1.88-1.78 (m, 3H), 1.73 (d, J = 1.2 Hz, 4H), 1.32-1.16 (m, 2H) | |
| 176 | ¹H NMR (400 MHz, DMSO-d₆) δ 10.96 (s, 1H), 10.12-10.00 (m, 1H), | B |
| | 8.92-8.80 (m, 1H), 8.72 (d, J = 8.8 Hz, 1H), 8.72-8.64 (m, 1H), 8.00 | |
| | (d, J = 8.8 Hz, 1H), 7.92-7.72 (m, 2H), 7.56-7.44 (m, 1H), 7.12-7.02 | |
| | (m, 1H), 7.02-6.84 (m, 2H), 5.10 (dd, J = 4.8, 13.2 Hz, 1H), 4.56 (s, | |
| | 2H), 4.52-4.44 (m, 1H), 4.28 (d, J = 17.2 Hz, 1H), 4.16-4.08 (m, 1H), | |
| | 3.32-3.24 (m, 1H), 3.06-2.80 (m, 7H), 2.76-2.56 (m, 4H), 2.56- | |
| | 2.52 (m, 2H), 2.48 (s, 3H), 2.44-2.32 (m, 2H), 2.04-1.92 (m, 2H), | |
| | 1.80-1.36 (m, 2H) | |
| 177 | ¹H NMR (400 MHz, DMSO-d₆) δ 11.00 (s, 1H), 9.85 (s, 1H), 8.78 (s, | B |
| | 1H), 8.61-8.50 (m, 1H), 8.46-8.37 (m, 1H), 8.00 (d, J = 2.4 Hz, 1H), | |
| | 7.81 (s, 1H), 7.67 (dd, J = 8.4, 14.4 Hz, 2H), 7.52 (dd, J = 2.4, 10.4 Hz, | |
| | 1H), 7.48-7.39 (m, 1H), 7.34-7.23 (m, 1H), 7.02-6.91 (m, 2H), 5.15- | |
| | 5.04 (m, 1H), 4.50-4.33 (m, 3H), 4.26 (d, J = 17.6 Hz, 1H), 3.47 (d, J = | |
| | 1.6 Hz, 2H), 3.39-3.35 (m, 1H), 3.13 (s, 4H), 2.89 (dd, J = 4.4, 13.2 | |
| | Hz, 1H), 2.82-2.66 (m, 6H), 2.60 (d, J = 2.4 Hz, 1H), 2.07 (s, 1H), | |
| | 2.03-1.88 (m, 3H), 1.73-1.58 (m, 2H) | |
| 178 | ¹H NMR (400 MHz, DMSO-d₆) δ 10.96 (s, 1H), 10.12-10.08 (m, 1H), | A |
| | 9.26-9.12 (m, 1H), 8.96 (s, 1H), 8.68 (d, J = 8.8 Hz, 1H), 8.64-8.56 | |
| | (m, 1H), 8.32-8.20 (m, 1H), 8.00-7.92 (m, 1H), 7.84-7.76 (m, 1H), | |
| | 7.68 (d, J = 8.8 Hz, 1H), 7.56 (d, J = 8.8 Hz, 1H), 7.48-7.36 (m, 1H), | |
| | 7.02-6.88 (m, 2H), 5.06 (dd, J = 5.2, 13.2 Hz, 1H), 4.80 (q, J = 6.10 | |
| | Hz, 1H), 4.56-4.44 (m, 2H), 4.28 (d, J = 17.6 Hz, 1H), 4.12 (d, J = | |
| | 17.2 Hz, 1H), 3.64 (d, J = 9.6 Hz, 2H), 3.24 (d, J = 9.6 Hz, 2H), 3.16 (s, | |
| | 3H), 3.12-3.00 (m, 4H), 2.96-2.88 (m, 1H), 2.56-2.52 (m, 3H), 2.48- | |
| | 2.32 (m, 2H), 2.24-2.16 (m, 1H), 2.00-1.92 (m, 1H), 1.92-1.80 (m, | |
| | 2H), 1.32-1.16 (m, 2H), 0.92 (d, J = 6.4 Hz, 3H) | |
| 179 | ¹H NMR (400 MHz, DMSO-d₆) δ 10.92-10.76 (m, 1H), 9.96 (s, 1H), | B |
| | 8.88-8.76 (m, 1H), 8.68 (d, J = 8.4 Hz, 1H), 8.48-8.40 (m, 1H), 7.96 | |
| | (s, 1H), 7.84 (s, 1H), 7.72 (d, J = 8.4 Hz, 1H), 7.56-7.52 (m, 1H), 7.48 | |
| | (d, J = 8.4 Hz, 1H), 7.40 (d, J = 7.6 Hz, 1H), 7.00-6.92 (m, 1H), 6.84 | |
| | (d, J = 8.0 Hz, 2H), 4.52-4.24 (m, 4H), 3.80-3.68 (m, 1H), 3.40-3.24 | |
| | (m, 2H), 3.20-2.76 (m, 9H), 2.72-2.60 (m, 2H), 2.48 (s, 3H), 2.24- | |
| | 2.16 (m, 1H), 2.12-1.88 (m, 3H), 1.60-1.40 (m, 2H) | |
| 180 | ¹H NMR (400 MHz, DMSO-d₆) δ 0.83-1.18 (m, 5H), 1.39-1.59 (m, | A |
| | 2H), 1.82-2.06 (m, 4H), 2.55-2.97 (m, 7H), 3.01-3.31 (m, 10H), 3.58- | |
| | 3.68 (m, 1H), 4.39 (s, 2H), 5.29 (dd, J=12, 4.75 Hz, 1H), 6.76-7.04 (m, | |
| | 5H), 7.44-7.60 (m, 2H), 7.72 (d, J=8.64 Hz, 1H), 7.83 (s, 1H), 8.37-8.48 | |
| | (m, 1H), 8.68 (d, J-8.76 Hz, 1H), 8.82 (s, 1H), 9.16-9.38 (m, 1H), 9.88- | |
| | 10.08 (m, 1H), 11.08 (s, 1H) | |
| 181 | ¹H NMR (400 MHz, DMSO-d₆) δ 10.96 (s, 1H), 10.00-9.92 (m, 1H), | B |
| | 8.84 (s, 1H), 8.60 (d, J = 8.4 Hz, 1H), 8.28 (dd, J = 6.0, 7.2 Hz, 1H), | |
| | 7.80-7.72 (m, 2H), 7.60 (d, J = 8.4 Hz, 1H), 7.52 (dd, J = 2.8, 10.4 Hz, | |
| | 1H), 7.44 (d, J = 8.4 Hz, 1H), 6.96-6.92 (m, 2H), 6.84 (d, J = 8.8 Hz, | |
| | 1H), 5.12-5.02 (m, 1H), 4.80-4.72 (m, 1H), 4.52-4.44 (m, 2H), 4.32- | |
| | 4.22 (m, 1H), 4.16-4.04 (m, 1H), 3.06-2.84 (m, 4H), 2.84 (s, 3H), | |
| | 2.72-2.56 (m, 6H), 2.44 (s, 3H), 2.36-2.32 (m, 1H), 2.00-1.88 (m, | |
| | 3H), 1.44 (q, J = 10.0 Hz, 2H), 0.76 (d, J = 6.8 Hz, 3H) | |
| 182 | ¹H NMR (400 MHz, DMSO-d₆) δ 11.06 (s, 1H), 9.94 (s, 1H), 8.81 (s, | B |
| | 1H), 8.66 (d, J = 8.4 Hz, 1H), 8.44 (dd, J = 6.0, 7.2 Hz, 1H), 7.82 (s, | |
| | 1H), 7.71 (d, J = 8.4 Hz, 1H), 7.52 (dd, J = 2.4, 10.0 Hz, 1H), 7.46 (d, | |
| | J = 8.4 Hz, 1H), 6.99-6.89 (m, 2H), 6.87-6.81 (m, 2H), 6.67 (dd, J = | |
| | 1.6, 8.4 Hz, 1H), 5.25 (d, J = 12.4 Hz, 1H), 4.38 (s, 2H), 3.62 (d, J = | |
| | 11.2 Hz, 2H), 2.91-2.81 (m, 6H), 2.73-2.57 (m, 8H), 2.46 (s, 3H), | |
| | 2.39 (s, 1H), 2.00-1.88 (m, 3H), 1.62 (dd, J = 1.6, 10.7 Hz, 2H), 1.07- | |
| | 1.01 (m, 2H), 0.90-0.83 (m, 2H) | |
| 183 | ¹H NMR (400 MHz, DMSO-d₆) δ 10.78 (s, 1H), 10.16-9.91 (m, 2H), | B |
| | 8.89 (s, 1H), 8.76-8.66 (m, 2H), 8.31 (s, 1H), 8.17 (br d, J = 6.0 Hz, | |
| | 1H), 7.98 (br d, J = 7.0 Hz, 1H), 7.87 (br s, 1H), 7.77 (d, J = 8.5 Hz, | |
| | 1H), 7.54 (d, J = 8.8 Hz, 1H), 7.46-7.39 (m, 1H), 7.12 (br d, J = 6.3 | |
| | Hz, 1H), 7.03-6.89 (m, 2H), 4.40 (s, 2H), 4.21-4.12 (m, 2H), 3.81 (br | |
| | s, 2H), 3.68 (br d, J = 9.9 Hz, 2H), 3.56 (br s, 1H), 3.33-3.17 (m, 4H), | |
| | 3.12-3.03 (m, 2H), 2.91-2.74 (m, 4H), 2.52 (br s, 3H), 2.28 (br d, J = | |
| | 10.4 Hz, 2H), 2.05-1.84 (m, 2H) | |
| 184 | ¹H NMR (400 MHz, DMSO-d₆) δ 10.96 (s, 1H), 10.04 (s, 1H), 8.84 (s, | A |
| | 1H), 8.72 (d, J = 8.8 Hz, 1H), 8.68 (dd, J = 5.6, 7.6 Hz, 1H), 7.96 (d, J = | |
| | 8.8 Hz, 1H), 7.76 (dd, J = 2.4, 9.6 Hz, 1H), 7.52-7.48 (m, 1H), 7.36 (d, | |
| | J = 8.0 Hz, 1H), 7.12-7.02 (m, 1H), 6.88 (d, J = 8.8 Hz, 1H), 6.60 (t, | |
| | J = 8.0 Hz, 1H), 5.02 (dd, J = 5.2, 13.4 Hz, 1H), 4.52 (s, 2H), 4.48-4.36 | |
| | (m, 1H), 4.24 (d, J = 16.8 Hz, 1H), 4.08 (s, 2H), 4.00-3.92 (m, 2H), | |
| | 2.96-2.80 (m, 5H), 2.76-2.52 (m, 6H), 2.48 (s, 3H), 2.48-2.28 (m, | |
| | 5H), 2.00-1.88 (m, 3H) | |
| 185 | ¹H NMR (400 MHz, DMSO-d₆) δ 11.00 (s, 1H), 9.96 (s, 1H), 8.80 (s, | C |
| | 1H), 8.68 (d, J = 8.8 Hz, 1H), 8.48-8.40 (m, 1H), 7.80 (s, 1H), 7.76- | |
| | 7.68 (m, 2H), 7.52 (d, J = 10.0 Hz, 1H), 7.48 (d, J = 8.8 Hz, 1H), 7.32 | |
| | (d, J = 8.0 Hz, 1H), 6.96 (m, 7.6 Hz, 1H), 6.84 (d, J = 8.4 Hz, 1H), 5.12 | |
| | (dd, J = 5.2, 13.2 Hz, 1H), 4.48-4.36 (m, 3H), 4.28 (d, J = 17.6 Hz, | |
| | 1H), 3.24 (s, 7H), 3.12 (d, J = 9.6 Hz, 4H), 2.96-2.84 (m, 2H), 2.72- | |
| | 2.60 (m, 3H), 2.48 (s, 3H), 2.16-1.96 (m, 3H), 1.76 (d, J = 8.4 Hz, 2H) | |
| 186 | ¹H NMR (400 MHz, DMSO-d₆) δ 10.36 (s, 1H), 9.94 (s, 1H), 8.81 (s, | B |
| | 1H), 8.66 (d, J = 8.4 Hz, 1H), 8.48-8.41 (m, 1H), 8.06 (d, J = 2.4 Hz, | |
| | 1H), 7.82 (s, 1H), 7.70 (d, J = 8.8 Hz, 1H), 7.55-7.43 (m, 3H), 6.96 (dt, | |
| | J = 2.4, 7.6 Hz, 1H), 6.84 (d, J = 8.8 Hz, 2H), 4.44-4.27 (m, 4H), 3.70 | |
| | (t, J = 6.8 Hz, 2H), 3.31 (s, 2H), 2.85 (d, J = 11.2 Hz, 6H), 2.70 (t, J = | |
| | 6.4 Hz, 4H), 2.54 (s, 1H), 2.46 (s, 3H), 2.01-1.80 (m, 2H), 1.55-1.35 | |
| | (m, 2H) | |
| 187 | ¹H NMR (400 MHz, DMSO-d₆) δ 8 10.96 (s, 1H), 10.03 (s, 1H), 8.81 (s, | A |
| | 1H), 8.79-8.72 (m, 1H), 8.42 (dd, J = 6.0, 7.4 Hz, 1H), 7.82 (s, 1H), | |
| | 7.69 (d, J = 8.8 Hz, 1H), 7.63-7.48 (m, 2H), 7.38 (d, J = 8.0 Hz, 1H), | |
| | 6.97 (dt, J = 2.8, 7.6 Hz, 2H), 6.60 (t, J = 8.0 Hz, 1H), 5.04 (dd, J = 5.2, | |
| | 13.2 Hz, 2H), 4.63 (d, J = 5.6 Hz, 2H), 4.47-4.36 (m, 3H), 4.26 (d, J = | |
| | 16.8 Hz, 1H), 4.10 (s, 2H), 3.97 (s, 2H), 3.16-2.78 (m, 6H), 2.68-2.54 | |
| | (m, 4H), 2.47-2.28 (m, 5H), 2.19-1.81 (m, 4H) | |
| 188 | ¹H NMR (400 MHz, DMSO-d₆) δ 10.96 (s, 1H), 9.96 (s, 1H), 8.80 (s, | A |
| | 1H), 8.68-8.56 (m, 2H), 8.04 (s, 1H), 7.956 (d, J = 8.8 Hz, 1H), 7.76 | |
| | (dd, J = 2.0, 9.6 Hz, 1H), 7.52-7.44 (m, 1H), 7.36 (d, J = 8.0 Hz, 1H), | |
| | 7.06 (dt, J = 2.4, 7.6 Hz, 1H), 7.02-6.96 (m, 1H), 6.60 (t, J = 8.0 Hz, | |
| | 1H), 4.96 (s, 1H), 4.52 (s, 2H), 4.48-4.40 (m, 1H), 4.24 (d, J = 16.8 | |
| | Hz, 1H), 4.16-4.08 (m, 2H), 3.96 (s, 2H), 3.24-2.96 (m, 4H), 2.96- | |
| | 2.84 (m, 2H), 2.64-2.52 (m, 6H), 2.48-2.32 (m, 4H), 2.08-1.88 (m, | |
| | 3H) | |
| 189 | ¹H NMR (400 MHz, DMSO-d₆) δ 10.96 (s, 1H), 9.99 (s, 1H), 8.82 (s, | A |
| | 1H), 8.73 (d, J = 8.4 Hz, 1H), 8.45 (dd, J = 6.0, 7.4 Hz, 1H), 8.16 (s, | |
| | 1H), 7.82 (s, 1H), 7.72 (d, J = 8.4 Hz, 1H), 7.58-7.47 (m, 3H), 7.07- | |
| | 7.02 (m, 2H), 6.97 (dt, J = 2.4, 7.5 Hz, 1H), 6.82 (d, J = 8.4 Hz, 1H), | |
| | 5.05 (dd, J = 5.2, 13.3 Hz, 1H), 4.43-4.28 (m, 3H), 4.24-4.15 (m, | |
| | 1H), 3.88 (d, J = 12.4 Hz, 2H), 3.01 (d, J = 10.4 Hz, 2H), 2.97-2.75 | |
| | (m, 4H), 2.70-2.62 (m, 1H), 2.52-2.51 (m, 3H), 2.40-2.33 (m, 1H), | |
| | 2.29-2.20 (m, 2H), 2.08 (t, J = 10.0 Hz, 2H), 2.00-1.91 (m, 1H), 1.81 | |
| | (d, J = 10.0 Hz, 3H), 1.74-1.61 (m, 4H), 1.25-1.13 (m, 2H) | |
| 190 | ¹H NMR (400 MHz, DMSO-d₆) δ 10.34 (s, 1H), 9.94 (s, 1H), 8.79 (s, | A |
| | 1H), 8.66 (d, J = 8.4 Hz, 1H), 8.47-8.42 (m, 1H), 8.01 (d, J = 2.4 Hz, | |
| | 1H), 7.82 (s, 1H), 7.71 (d, J = 8.8 Hz, 1H), 7.55-7.50 (m, 1H), 7.50- | |
| | 7.45 (m, 2H), 7.00-6.93 (m, 1H), 6.84 (d, J = 8.4 Hz, 1H), 6.40 (d, J = | |
| | 8.8 Hz, 1H), 4.38 (s, 2H), 4.06 (t, J = 8.0 Hz, 2H), 3.69 (t, J = 6.8 Hz, | |
| | 2H), 3.64-3.60 (m, 2H), 2.99-2.94 (m, 1H), 2.84 (s, 4H), 2.72-2.64 | |
| | (m, 5H), 2.56 (s, 3H), 2.46 (s, 3H) | |
| 191 | ¹H NMR (400 MHz, DMSO-d₆) δ 11.00 (s, 1H), 9.93 (s, 1H), 8.81 (s, | B |
| | 1H), 8.76-8.68 (m, 1H), 8.48-8.40 (m, 1H), 8.30 (s, 1H), 7.82 (s, 1H), | |
| | 7.71 (d, J = 8.4 Hz, 1H), 7.65 (d, J = 8.0 Hz, 1H), 7.52 (dd, J = 2.4, 10.4 | |
| | Hz, 1H), 7.28 (d, J = 8.4 Hz, 1H), 6.97 (dt, J = 2.4, 7.6 Hz, 1H), 6.67 (s, | |
| | 1H), 5.10 (dd, J = 5.2, 13.2 Hz, 1H), 4.45-4.35 (m, 3H), 4.26 (d, J = | |
| | 17.6 Hz, 1H), 3.01 (s, 4H), 2.95-2.90 (m, 1H), 2.78 (s, 3H), 2.66 (s, | |
| | 5H), 2.54 (s, 1H), 2.48 (s, 3H), 2.44 (d, J = 7.2 Hz, 2H), 2.30 (s, 3H), | |
| | 2.03-1.89 (m, 3H), 1.74-1.61 (m, 2H) | |
| 192 | ¹H NMR (400 MHz, DMSO-d₆) δ 10.57 (s, 1H), 9.94 (s, 1H), 8.83- | A |
| | 8.75 (m, 2H), 8.70-8.62 (m, 1H), 8.49-8.40 (m, 1H), 8.03 (d, J = 2.4 | |
| | Hz, 1H), 7.82 (s, 1H), 7.73-7.68 (m, 1H), 7.55-7.45 (m, 2H), 7.23 (s, | |
| | 1H), 6.99-6.92 (m, 2H), 6.87-6.81 (m, 1H), 4.38 (s, 2H), 3.96-3.88 | |
| | (m, 4H), 2.87 (s, 4H), 2.78 (d, J = 7.2 Hz, 2H), 2.72 (d, J = 10.8 Hz, | |
| | 2H), 2.63-2.55 (m, 3H), 2.47 (s, 3H), 2.44 (s, 3H), 2.40-2.30 (m, 2H), | |
| | 1.92-1.83 (m, 2H), 1.81-1.70 (m, 1H), 1.53-1.38 (m, 2H) | |
| 193 | ¹H NMR (400 MHz, DMSO-d₆) δ 11.01 (s, 1H), 9.93 (s, 1H), 8.83- | C |
| | 8.77 (m, 1H), 8.66 (d, J = 8.8 Hz, 1H), 8.48-8.41 (m, 1H), 7.82 (s, 1H), | |
| | 7.70 (dd, J = 6.4, 8.0 Hz, 2H), 7.60 (d, J = 7.6 Hz, 1H), 7.52 (dd, J = | |
| | 2.8, 10.0 Hz, 1H), 7.45 (d, J = 9.2 Hz, 1H), 6.96 (dt, J = 3.2, 7.6 Hz, | |
| | 1H), 6.84 (d, J = 8.4 Hz, 1H), 5.17-5.08 (m, 1H), 4.52-4.42 (m, 1H), | |
| | 4.38 (s, 2H), 4.34-4.27 (m, 1H), 3.07 ( dd, J = 3.6, 7.2 Hz, 4H), 2.97- | |
| | 2.87 (m, 1H), 2.70-2.60 (m, 3H), 2.59-2.54 (m, 1H), 2.46 (s, 3H), | |
| | 2.45-2.39 (m, 2H), 2.39 ( s, 2H), 2.06-1.98 (m, 1H), 1.92-1.85 (m, | |
| | 2H), 1.85-1.77 (m, 2H), 1.77-1.64 (m, 4H) | |
| 194 | ¹H NMR (400 MHz, DMSO-d₆) δ 10.64 (s, 1H), 10.00 (bs, 1H), 8.81 (s, | A |
| | 1H), 8.68 (d, J = 8.8 Hz, 1H), 8.44 (dd, J = 6.0, 7.2 Hz, 1H), 7.86-7.81 | |
| | (m, 2H), 7.71 (d, J = 8.8 Hz, 1H), 7.55-7.45 (m, 3H), 6.97 (dt, J = 2.8, | |
| | 7.6 Hz, 1H), 6.91-6.81 (m, 2H), 6.57-6.51 (m, 1H), 4.39 (s, 2H), 4.36- | |
| | 4.28 (m, 2H), 3.76 (t, J = 6.0 Hz, 2H), 3.71-3.57 (m, 1H), 3.38-3.25 | |
| | (m, 8H), 3.14-3.01 (m, 3H), 2.90-2.72 (m, 5H), 2.20-1.98 (m, 1H), | |
| | 1.91 (d, J = 11.6 Hz, 2H), 1.55-1.36 (m, 2H) | |
| 195 | ¹H NMR (400 MHz, DMSO-d₆) δ 10.92 (s, 1H), 9.97-9.87 (m, 1H), | A |
| | 8.78 (s, 1H), 8.65 (d, J = 8.4 Hz, 1H), 8.47-8.40 (m, 1H), 8.13 (s, 1H), | |
| | 7.81 (s, 1H), 7.70 (d, J = 8.8 Hz, 1H), 7.51 (dd, J = 2.8, 10.0 Hz, 1H), | |
| | 7.49-7.45 (m, 2H), 6.96 (dt, J = 2.8, 7.6 Hz, 1H), 6.84 (d, J = 8.8 Hz, | |
| | 1H), 6.54-6.41 (m, 2H), 5.06-4.97 (m, 1H), 4.38 (s, 2H), 4.33-4.25 | |
| | (m, 1H), 4.21-4.12 (m, 1H), 3.69-3.54 (m, 4H), 2.95-2.88 (m, 1H), | |
| | 2.85 ( s, 4H), 2.63-2.52 (m, 4H), 2.46 (s, 3H), 2.38-2.33 (m, 1H), | |
| | 2.31-2.26 (m, 1H), 2.26-2.15 (m, 2H), 2.01-1.85 (m, 3H), 1.79- | |
| | 1.68 (m, 2H), 1.58-1.43 (m, 3H), 1.05-0.88 (m, 2H) | |
| 196 | ¹H NMR (400 MHz, DMSO-d₆) δ 10.98 (s, 1H), 9.93 (s, 1H), 8.79 (s, | A |
| | 1H), 8.65 (d, J = 8.4 Hz, 1H), 8.44 (dd, J = 6.0, 7.2 Hz, 1H), 7.81 (s, | |
| | 1H), 7.70 (d, J = 8.4 Hz, 1H), 7.51 (dd, J = 2.4, 10.4 Hz, 1H), 7.46 (d, J = | |
| | 8.4 Hz, 2H), 7.17 (t, J = 8.0 Hz, 1H), 6.96 (dt, J = 2.4, 7.4 Hz, 1H), | |
| | 6.83 (d, J = 8.4 Hz, 1H), 5.07 (dd, J = 5.2, 13.2 Hz, 1H), 4.47 (d, J = | |
| | 17.2 Hz, 1H), 4.38 (s, 2H), 4.30 (d, J = 16.8 Hz, 1H), 3.10 (s, 4H), | |
| | 2.97-2.77 (m, 6H), 2.69-2.53 (m, 3H), 2.53-2.51 (m, 1H), 2.45 (s, 3H), | |
| | 2.43-2.39 (m, 1H), 2.20 (d, J = 6.4 Hz, 2H), 2.01-1.93 (m, 1H), 1.75- | |
| | 1.68 (m, 2H), 1.62 (d, J = 5.6 Hz, 4H), 1.47 (d, J = 0.8 Hz, 3H), 1.11 (d, | |
| | J = 9.6 Hz, 4H) | |
| 197 | ¹H NMR (400 MHz, DMSO-d₆) δ 10.83 (s, 1H), 10.03 (s, 1H), 8.84 (s, | B |
| | 1H), 8.70 (d, J = 8.4 Hz, 1H), 8.55 (d, J = 8.0 Hz, 2H), 8.10 (s, 1H), | |
| | 7.91 (s, 1H), 7.74 (d, J = 8.4 Hz, 2H), 7.51 (d, J = 8.8 Hz, 1H), 7.43- | |
| | 7.36 (m, 1H), 7.23 (d, J = 4.4 Hz, 1H), 7.03-6.85 (m, 2H), 4.38 (s, 2H), | |
| | 4.30 (d, J = 12.4 Hz, 2H), 3.83 (s, 4H), 3.32-3.18 (m, 5H), 3.15-3.00 | |
| | (m, 5H), 2.84 (s, 2H), 2.58-2.54 (m, 2H), 2.25 (d, J = 10.4 Hz, 2H), | |
| | 1.74 (d, J = 9.6 Hz, 2H) | |
| 198 | ¹H NMR (400 MHz, DMSO-d₆) δ 11.01 (s, 1H), 10.05 (s, 1H), 8.87 (s, | B |
| | 1H), 8.71 (d, J = 8.4 Hz, 1H), 8.67-8.62 (m, 1H), 8.28-8.19 (m, 1H), | |
| | 7.91-7.83 (m, 1H), 7.76 (d, J = 8.4 Hz, 1H), 7.55-7.49 (m, 1H), 7.46 | |
| | (d, J = 7.6 Hz, 1H), 7.39-7.29 (m, 2H), 7.25-7.18 (m, 1H), 6.97-6.87 | |
| | (m, 1H), 5.18-5.09 (m, 1H), 4.53-4.45 (m, 1H), 4.43-4.37 (m, 2H), | |
| | 4.36-4.28 (m, 1H), 3.60 (d, J = 9.2 Hz, 4H), 3.27-3.19 (m, 5H), 3.00- | |
| | 2.75 (m, 5H), 2.67-2.55 (m, 3H), 2.35-2.20 (m, 3H), 2.09-1.84 (m, | |
| | 4H | |
| 199 | ¹H NMR (400 MHz, DMSO-d₆) δ 11.00 (s, 1H), 10.02 (s, 1H), 8.84 (s, | B |
| | 1H), 8.55 (d, J = 8.4 Hz, 1H), 8.41 (t, J = 6.7 Hz, 1H), 8.14 (s, 1H), 7.83 | |
| | (s, 1H), 7.68 (dd, J = 8.4, 18.8 Hz, 3H), 7.53 (dd, J = 2.4, 10.0 Hz, 1H), | |
| | 7.29 (d, J = 8.0 Hz, 1H), 7.07 (d, J = 8.4 Hz, 1H), 6.99 (dt, J = 2.4, 7.2 | |
| | Hz, 1H), 5.10 (dd, J = 4.8, 13.2 Hz, 1H), 4.50-4.34 (m, 3H), 4.26 (d, | |
| | J = 17.6 Hz, 1H), 4.05 (s, 2H), 3.48 (s, 3H), 2.99-2.86 (m, 5H), 2.81- | |
| | 2.72 (m, 5H), 2.64 (s, 6H), 2.57 (d, J = 0.8 Hz, 1H), 2.48-2.41 (m, 1H), | |
| | 2.03-1.89 (m, 3H), 1.77-1.62 (m, 2H) | |
| 200 | ¹H NMR (400 MHz, DMSO-d₆) δ 10.95 (s, 1H), 9.94 (s, 1H), 8.79 (s, | A |
| | 1H), 8.66 (d, J = 8.4 Hz, 1H), 8.40 (d, J = 6.8 Hz, 1H), 7.84 (s, 1H), | |
| | 7.72 (t, J = 9.2 Hz, 2H), 7.65 (d, J = 9.2 Hz, 1H), 7.46 (d, J = 8.8 Hz, | |
| | 1H), 7.33-7.26 (m, 1H), 6.97-6.87 (m, 2H), 6.84 (d, J = 8.4 Hz, 1H), | |
| | 5.07 (dd, J = 4.8, 13.2 Hz, 1H), 4.49-4.34 (m, 4H), 4.26 (d, J = 17.2 | |
| | Hz, 1H), 4.09 (d, J = 17.2 Hz, 1H), 2.95 (s, 3H), 2.85 (s, 4H), 2.64- | |
| | 2.52 (m, 5H), 2.46 (s, 3H), 2.36 (dd, J = 4.2, 13.2 Hz, 1H), 2.25 (d, J = | |
| | 5.6 Hz, 2H), 2.00-1.86 (m, 2H), 1.82 (d, J = 12.8 Hz, 2H), 1.10 (d, J = | |
| | 11.6 Hz, 2H) | |
| 201 | ¹H NMR (400 MHz, DMSO-d₆) δ 11.00 (s, 1H), 10.01-9.91 (m, 1H), | B |
| | 8.80 (s, 1H), 8.67 (d, J = 8.4 Hz, 1H), 8.40 (d, J = 6.8 Hz, 1H), 7.84 (s, | |
| | 1H), 7.72 (d, J = 8.4 Hz, 1H), 7.66 (d, J = 8.4 Hz, 2H), 7.49 (d, J = 8.0 | |
| | Hz, 1H), 7.33-7.25 (m, 2H), 6.94 (t, J = 6.8 Hz, 1H), 6.89-6.82 (m, | |
| | 1H), 5.11 (dd, J = 4.8, 13.2 Hz, 1H), 4.39 (s, 3H), 4.30-4.23 (m, 1H), | |
| | 3.54-3.45 (m, 4H), 3.37 (s, 2H), 2.98-2.85 (m, 5H), 2.85-2.73 (m, | |
| | 5H), 2.61 (s, 2H), 2.56 (d, J = 13.2 Hz, 2H), 2.04-1.93 (m, 2H), 1.72 | |
| | (d, J = 2.4 Hz, 2H) | |
| 202 | ¹H NMR (400 MHz, DMSO-d₆) δ 10.94 (s, 1H), 9.92 (s, 1H), 8.79 (s, | A |
| | 1H), 8.72 (d, J = 8.4 Hz, 1H), 8.46-8.39 (m, 1H), 7.81 (s, 1H), 7.72 | |
| | (dd, J = 8.8, 16.0 Hz, 2H), 7.51 (dd, J = 2.4, 10.0 Hz, 1H), 6.96 (d, J = | |
| | 2.4 Hz, 1H), 6.89 (d, J = 9.2 Hz, 1H), 6.66 (s, 1H), 5.07 (dd, J = 5.2, | |
| | 13.2 Hz, 1H), 4.48-4.33 (m, 4H), 4.26 (d, J = 17.2 Hz, 1H), 4.09 (d, | |
| | J = 17.2 Hz, 1H), 2.99 (s, 4H), 2.96-2.86 (m, 3H), 2.64-2.53 (m, 2H), | |
| | 2.51 (s, 1H), 2.46 (s, 5H), 2.36 (dd, J = 4.8, 13.2 Hz, 1H), 2.28 (s, 3H), | |
| | 2.21 (d, J = 6.4 Hz, 2H), 2.00-1.86 (m, 2H), 1.82 (d, J = 12.8 Hz, 2H), | |
| | 1.09 (d, J = 10.4 Hz, 2H) | |
| 203 | ¹H NMR (400 MHz, DMSO-d₆) δ 10.65-10.58 (m, 1H), 10.05 (s, 1H), | A |
| | 9.12-8.95 (m, 1H), 8.82 (s, 1H), 8.78-8.69 (m, 1H), 8.41 (s, 1H), 7.85 | |
| | (s, 1H), 7.76-7.66 (m, 2H), 7.59-7.45 (m, 2H), 7.43 (s, 1H), 6.99 (dt, | |
| | J = 2.8, 7.5 Hz, 1H), 6.92 (d, J = 8.4 Hz, 1H), 6.42 (br s, 1H), 4.50- | |
| | 4.24 (m, 4H), 3.83-3.60 (m, 4H), 3.23-2.99 (m, 6H), 2.88-2.71 (m, | |
| | 4H), 2.56 (s, 3H), 2.26 (s, 3H), 2.05-1.85 (m, 6H), 1.53-1.40 (m, 2H) | |
| 204 | ¹H NMR (400 MHz, DMSO-d₆) δ 10.83 (s, 1H), 9.94 (s, 1H), 8.80 (s, | A |
| | 1H), 8.66 (d, J = 8.4 Hz, 1H), 8.49-8.40 (m, 1H), 7.95 (d, J = 2.0 Hz, | |
| | 1H), 7.82 (s, 1H), 7.70 (d, J = 8.4 Hz, 1H), 7.55-7.41 (m, 3H), 7.01- | |
| | 6.93 (m, 1H), 6.84 (d, J = 8.4 Hz, 1H), 6.49 (d, J = 8.4 Hz, 1H), 4.38 (s, | |
| | 2H), 4.13 (dd, J = 9.6, 18.8 Hz, 2H), 4.06-3.96 (m, 2H), 3.76 (dd, J = | |
| | 4.8, 12.4 Hz, 1H), 2.96 (s, 2H), 2.84 (d, J = 4.2 Hz, 4H), 2.73-2.63 (m, | |
| | 5H), 2.54 (d, J = 2.4 Hz, 1H), 2.46 (s, 3H), 2.26-2.13 (m, 1H), 1.98 (d, | |
| | J = 3.6 Hz, 1H) | |
| 205 | ¹H NMR (400 MHz, DMSO-d₆) δ 10.63 (s, 1H), 10.04-9.96 (m, 1H), | A |
| | 9.44-9.25 (m, 1H), 8.83-8.78 (m, 1H), 8.72-8.64 (m, 1H), 8.49- | |
| | 8.41 (m, 1H), 7.89-7.81 (m, 1H), 7.72 (br d, J = 8.8 Hz, 2H), 7.61- | |
| | 7.49 (m, 2H), 7.48-7.39 (m, 1H), 7.00 (dt, J = 2.4, 7.6 Hz, 1H), 6.90 | |
| | (d, J = 8.8 Hz, 1H), 6.50-6.37 (m, 1H), 4.51-4.17 (m, 4H), 3.79-3.57 | |
| | (m, 4H), 3.26-3.04 (m, 9H), 2.89-2.72 (m, 4H), 2.27 (s, 3H), 2.14- | |
| | 2.04 (m, 1H), 1.96-1.86 (m, 2H), 1.37 (br d, J = 3.6 Hz, 2H) | |
| 206 | ¹H NMR (400 MHz, DMSO-d₆) δ 11.05 (s, 1H), 9.93 (s, 1H), 8.80 (s, | A |
| | 1H), 8.72 (d, J = 8.4 Hz, 1H), 8.44 (t, J = 6.4 Hz, 1H), 7.81 (s, 1H), 7.70 | |
| | (d, J = 8.4 Hz, 1H), 7.55-7.47 (m, 1H), 7.00-6.85 (m, 3H), 6.69-6.59 | |
| | (m, 2H), 5.26 (dd, J = 4.8, 12.4 Hz, 1H), 4.59 (m, 1H), 4.38 (s, 2H), | |
| | 3.56 (d, J = 10.4 Hz, 2H), 3.01 (s, 4H), 2.93-2.82 (m, 1H), 2.69-2.56 | |
| | (m, 4H), 2.47 (s, 7H), 2.31-2.23 (m, 5H), 2.03-1.93 (m, 1H), 1.84 (d, | |
| | J = 10.4 Hz, 2H), 1.72-1.61 (m, 1H), 1.45 (d, J = 6.8 Hz, 6H), 1.35- | |
| | 1.21 (m, 2H) | |
| 207 | ¹H NMR (400 MHz, DMSO-d₆) δ 11.00 (s, 1H), 9.98 (s, 1H), 8.81 (s, | A |
| | 1H), 8.67 (d, J = 8.4 Hz, 1H), 8.44 (dd, J = 6.0, 7.3 Hz, 1H), 7.82 (s, | |
| | 1H), 7.71 (d, J = 8.4 Hz, 1H), 7.56-7.45 (m, 2H), 7.32 (t, J = 7.6 Hz, | |
| | 1H), 7.06 (d, J = 7.2 Hz, 1H), 6.97 (dt, J = 2.4, 7.5 Hz, 1H), 6.87 (d, J = | |
| | 8.4 Hz, 1H), 6.54 (d, J = 8.0 Hz, 1H), 5.11 (dd, J = 5.0, 13.3 Hz, 1H), | |
| | 4.47-4.37 (m, 3H), 4.28 (d, J = 16.8 Hz, 1H), 4.08-4.01 (m, 2H), 3.94- | |
| | 3.87 (m, 2H), 3.15-2.79 (m, 10H), 2.65-2.52 (m, 3H), 2.48 (s, 3H), | |
| | 2.44-2.35 (m, 3H), 2.00 (d, J = 8.0 Hz, 3H) | |
| 208 | ¹H NMR (400 MHz, DMSO-d₆) δ 10.73 (s, 1H), 9.93 (s, 1H), 9.63 (s, | B |
| | 1H), 8.79 (s, 1H), 8.65 (d, J = 8.8 Hz, 1H), 8.44 (dd, J = 6.0, 7.2 Hz, | |
| | 1H), 7.81 (s, 1H), 7.70 (d, J = 8.4 Hz, 1H), 7.52 (dd, J = 2.4, 10.0 Hz, | |
| | 1H), 7.48-7.42 (m, 2H), 6.99-6.92 (m, 1H), 6.86-6.78 (m, 2H), 6.72 | |
| | (dd, J = 2.0, 9.2 Hz, 1H), 4.38 (s, 2H), 3.74 (d, J = 12.0 Hz, 2H), 3.26- | |
| | 3.19 (m, 2H), 2.83 (s, 4H), 2.74 (d, J = 5.2 Hz, 1H), 2.71-2.63 (m, 7H), | |
| | 2.71-2.63 (m, 1H), 2.60 (d, J = 7.2 Hz, 1H), 2.45 (s, 3H), 1.87 (d, J = | |
| | 10.0 Hz, 2H), 1.60-1.45 (m, 2H) | |
| 209 | ¹H NMR (400 MHz, DMSO-d₆) δ 11.09 (s, 1H), 9.93 (s, 1H), 8.81 (s, | A |
| | 1H), 8.73 (d, J = 8.4 Hz, 1H), 8.44 (dd, J = 6.4, 7.2 Hz, 1H), 7.81 (s, | |
| | 1H), 7.70 (d, J = 8.4 Hz, 1H), 7.52 (dd, J = 2.8, 9.6 Hz, 1H), 7.00-6.93 | |
| | (m, 2H), 6.88 (dd, J = 8.4, 12.4 Hz, 2H), 6.67 (s, 1H), 5.35 (dd, J = 5.2, | |
| | 12.4 Hz, 1H), 4.38 (s, 2H), 3.64 (s, 3H), 3.16-3.10 (m, 2H), 3.01 (s, | |
| | 4H), 2.76-2.57 (m, 6H), 2.54 (s, 3H), 2.47 (s, 3H), 2.29 (s, 5H), 2.03- | |
| | 1.96 (m, 1H), 1.86 (d, J = 11.2 Hz, 2H), 1.75-1.64 (m, 1H), 1.41-1.28 | |
| | (m, 2H) | |
| 210 | ¹H NMR (400 MHz, DMSO-d₆) δ 10.81 (s, 1H), 9.93 (s, 1H), 8.80 (s, | A |
| | 1H), 8.72 (d, J = 8.4 Hz, 1H), 8.44 (t, J = 6.0 Hz, 1H), 8.16 (s, 1H), 7.90 | |
| | (s, 1H), 7.81 (s, 1H), 7.69 (d, J = 8.4 Hz, 1H), 7.51 (d, J = 9.6 Hz, 1H), | |
| | 7.36 (d, J = 8.4 Hz, 1H), 7.00-6.91 (m, 1H), 6.65 (s, 1H), 6.35 (d, J = | |
| | 8.4 Hz, 1H), 4.37 (s, 2H), 4.02 (t, J = 7.2 Hz, 3H), 3.72 (dd, J = 4.4, | |
| | 11.6 Hz, 2H), 3.62-3.56 (m, 2H), 2.99 (s, 6H), 2.65 (d, J = 7.2 Hz, | |
| | 3H), 2.55-2.52 (m, 2H), 2.46 (s, 3H), 2.28 (s, 3H), 2.16 (d, J = 9.2 Hz, | |
| | 1H), 1.96 (dd, J = 4.0, 7.6 Hz, 1H) | |
| 211 | ¹H NMR (400 MHz, DMSO-d₆) δ 11.09 (s, 1H), 9.99 (s, 1H), 8.82 (s, | A |
| | 1H), 8.68 (d, J = 8.4 Hz, 1H), 8.48-8.40 (m, 1H), 7.82 (s, 1H), 7.71 (d, | |
| | J = 8.8 Hz, 1H), 7.56-7.45 (m, 2H), 6.97 (dt, J = 2.4, 7.4 Hz, 1H), 6.87 | |
| | (d, J = 8.4 Hz, 1H), 6.79 (d, J = 8.4 Hz, 1H), 6.23 (t, J = 8.4 Hz, 1H), | |
| | 5.29 (dd, J = 5.2, 12.8 Hz, 1H), 4.74 (td, J = 6.4, 13.2 Hz, 1H), 4.39 (s, | |
| | 2H), 3.91 (s, 2H), 3.81-3.75 (m, 2H), 3.16-2.81 (m, 8H), 2.70-2.55 | |
| | (m, 4H), 2.48 (s, 3H), 2.39 (s, 3H), 2.18-1.84 (m, 4H), 1.40 (d, J = 6.8 | |
| | Hz, 6H) | |
| 212 | ¹H NMR (400 MHz, DMSO-d₆) δ 11.07 (s, 1H), 9.98 (s, 1H), 8.82 (s, | B |
| | 1H), 8.73 (d, J = 8.4 Hz, 1H), 8.48-8.40 (m, 1H), 8.20-8.15 (m, 1H), | |
| | 7.83 (s, 1H), 7.72 (d, J = 8.4 Hz, 1H), 7.58-7.49 (m, 2H), 7.00-6.89 | |
| | (m, 2H), 6.86-6.80 (m, 2H), 6.67 (dd, J = 2.0, 8.4 Hz, 1H), 5.26 (dd, J = | |
| | 5.2, 12.4 Hz, 1H), 4.39 (s, 2H), 3.63 (d, J = 8.0 Hz, 2H), 3.06 (d, J = | |
| | 8.0 Hz, 2H), 2.86 (td, J = 3.2, 6.8 Hz, 2H), 2.70-2.57 (m, 6H), 2.43- | |
| | 2.33 (m, 3H), 1.89 (d, J = 11.6 Hz, 4H), 1.78-1.58 (m, 7H), 1.08-1.00 | |
| | (m, 2H), 0.87 (d, J = 1.6 Hz, 2H) | |
| 213 | ¹H NMR (400 MHz, DMSO-d₆) δ 11.10 (s, 1H), 9.94 (s, 1H), 8.79 (s, | A |
| | 1H), 8.66 (d, J = 8.4 Hz, 1H), 8.40 (d, J = 6.8 Hz, 1H), 7.84 (s, 1H), | |
| | 7.72 (d, J = 9.2 Hz, 1H), 7.66 (d, J = 9.2 Hz, 1H), 7.47 (d, J = 8.0 Hz, | |
| | 1H), 7.30 (t, J = 7.6 Hz, 1H), 7.00-6.82 (m, 5H), 5.35 (dd, J = 4.8, 12.4 | |
| | Hz, 1H), 4.39 (s, 2H), 3.64 (s, 3H), 3.13 (d, J = 10.4 Hz, 2H), 2.85 (s, | |
| | 5H), 2.76-2.62 (m, 5H), 2.61-2.54 (m, 3H), 2.47 (s, 3H), 2.29 (d, J = | |
| | 4.8 Hz, 2H), 1.99 (dd, J = 5.6, 7.2 Hz, 1H), 1.85 (d, J = 11.6 Hz, 2H), | |
| | 1.68 (d, J = 2.4 Hz, 1H), 1.43-1.27 (m, 2H) | |
| 214 | ¹H NMR (400 MHz, DMSO-d₆) δ 11.08 (s, 1H), 9.99 (s, 1H), 8.83 (s, | A |
| | 1H), 8.68 (d, J = 8.4 Hz, 1H), 8.41 (d, J = 6.8 Hz, 1H), 7.87 (s, 1H), | |
| | 7.73 (d, J = 8.4 Hz, 1H), 7.67 (d, J = 9.2 Hz, 1H), 7.50 (d, J = 8.8 Hz, | |
| | 1H), 7.36-7.28 (m, 1H), 6.99-6.85 (m, 4H), 6.65 (d, J = 8.8 Hz, 1H), | |
| | 5.28 (dd, J = 5.2, 12.8 Hz, 1H), 4.66-4.53 (m, 1H), 4.40 (s, 2H), 3.60 | |
| | (d, J = 11.2 Hz, 2H), 3.34 (s, 5H), 3.09-2.97 (m, 4H), 2.95-2.83 (m, | |
| | 2H), 2.74-2.53 (m, 5H), 2.49 (s, 3H), 2.03-1.93 (m, 1H), 1.86 (d, J = | |
| | 11.2 Hz, 2H), 1.45 (d, J = 6.8 Hz, 6H), 1.42-1.31 (m, 2H) | |
| 215 | ¹H NMR (400 MHz, DMSO-d₆) δ 11.00 (s, 1H), 9.91 (s, 1H), 8.88- | B |
| | 8.68 (m, 2H), 8.44 (dd, J = 7.2, 6.0 Hz, 1H), 7.82 (s, 1H), 7.75-7.61 | |
| | (m, 2H), 7.52 (dd, J = 10.2, 2.4 Hz, 1H), 7.29 (d, J = 8.4 Hz, 1H), 7.02- | |
| | 6.91 (m, 1H), 6.66 (s, 1H), 5.10 (dd, J = 13.2, 5.2 Hz, 1H), 4.47-4.33 | |
| | (m, 3H), 4.26 (d, J = 17.4 Hz, 1H), 3.89-3.74 (m, 1H), 3.54-3.45 (m, | |
| | 2H), 3.19-3.07 (m, 2H), 3.08-2.89 (m, 3H), 2.84-2.73 (m, 2H), 2.66- | |
| | 2.61 (m, 1H), 2.59-2.56 (m, 3H), 2.47-2.41 (m, 2H), 2.37 (s, 3H), | |
| | 2.16-2.06 (m, 2H), 2.02-1.88 (m, 3H), 1.80-1.57 (m, 4H). | |
| 216 | ¹H NMR (400 MHz, DMSO-d₆) δ 1.52 (d, J = 9.76 Hz, 2H), 1.79-1.98 | B |
| | (m, 3H), 2.08-2.21 (m, 1H), 2.31-2.43 (m, 1H), 2.46 (s, 3H), 2.57-2.94 | |
| | (m, 12H), 3.72-3.92 (m, 3H), 4.38 (s, 2H), 6.65-6.88 (m, 3H), 6.90-7.14 | |
| | (m, 2H), 7.41-7.56 (m, 2H), 7.70 (d, J = 8.52 Hz, 1H), 7.81 (s, 1H), 8.44 | |
| | (dd, J = 7.44, 6.07 Hz, 1H), 8.66 (d, J-8.64 Hz, 1H), 8.79 (s, 1H), 9.93 | |
| | (s, 1H), 10.81 (s, 1H) | |
| 217 | ¹H NMR (400 MHz, DMSO-d₆) δ 1.45-2.01 (m, 10H), 2.11-2.21 (m, | B |
| | 1H), 2.60-2.84 (m, 6H), 3.11-3.27 (m, 6H), 3.78-3.95 (m, 3H), 4.39 (s, | |
| | 2H), 6.71-7.13 (m, 5H), 7.52 (d, J = 8.00 Hz, 2H), 7.66-7.89 (m, 2H), | |
| | 8.44 (t, J-6.56 Hz, 1H), 8.68-8.88 (m, 2H), 9.99 (s, 1H), 10.82 (s, 1H) | |
| 218 | ¹H NMR (400 MHz, DMSO-d₆) δ 11.09-10.95 (m, 1H), 10.05-9.87 | B |
| | (m, 1H), 8.88-8.74 (m, 1H), 8.72-8.59 (m, 1H), 8.48-8.38 (m, 1H), | |
| | 7.82 (s, 1H), 7.74-7.65 (m, 1H), 7.56-7.39 (m, 2H), 7.30-7.14 (m, | |
| | 2H), 7.00-6.76 (m, 2H), 5.16-5.04 (m, 1H), 4.51-4.20 (m, 4H), 2.97- | |
| | 2.59 (m, 18H), 2.08-1.80 (m, 4H), 1.72-1.47 (m, 2H) | |
| 219 | ¹H NMR (400 MHz, DMSO-d₆) δ 10.80 (s, 1H), 9.91 (s, 1H), 8.80- | A |
| | 8.75 (m, 2H), 8.44-8.19 (m, 1H), 8.51-8.38 (m, 1H), 7.90 (d, J = 2.0 | |
| | Hz, 1H), 7.81 (s, 1H), 7.70 (d, J = 8.8 Hz, 1H), 7.52 (dd, J = 8.4, 2.4 Hz, | |
| | 1H), 7.37 (dd, J = 8.8, 2.4 Hz, 1H), 7.02-6.91 (m, 1H), 6.65 (s, 1H), | |
| | 6.36 (d, J = 8.4 Hz, 1H), 4.38 (s, 2H), 4.03 (t, J = 7.6 Hz, 2H), 3.77- | |
| | 3.67 (m, 1H), 3.67-3.55 (m, 2H), 3.05-2.84 (m, 4H), 2.72-2.61 (m, | |
| | 3H), 2.58-2.55 (m, 3H), 2.54-2.52 (m, 1H), 2.38-2.31 (m, 3H), | |
| | 2.21-1.96 (m, 6H), 1.64-1.52 (m, 2H) | |
| 220 | ¹H NMR (400 MHz, DMSO-d₆) δ 11.09 (s, 1H), 9.93 (s, 1H), 8.79 (s, | C |
| | 1H), 8.65 (d, J = 8.4 Hz, 1H), 8.47-8.40 (m, 1H), 7.81 (s, 1H), 7.70 (d, | |
| | J = 8.4 Hz, 1H), 7.52 (dd, J = 2.4, 10.0 Hz, 1H), 7.46 (d, J = 8.8 Hz, | |
| | 1H), 7.02-6.93 (m, 3H), 6.89 (d, J = 7.6 Hz, 1H), 6.84 (d, J = 8.8 Hz, | |
| | 1H), 5.36 (dd, J = 5.2, 12.4 Hz, 1H), 4.38 (s, 2H), 3.64 (s, 3H), 3.14- | |
| | 2.82 (m, 9H), 2.76-2.57 (m, 6H), 2.56-2.53 (m, 1H), 2.47 (s, 3H), | |
| | 2.03-1.90 (m, 3H), 1.72-1.60 (m, 2H) | |
| 221 | ¹H NMR (400 MHz, DMSO-d₆) δ 10.97 (s, 1H), 10.01 (s, 1H), 8.83 (s, | A |
| | 1H), 8.69 (d, J = 8.8 Hz, 1H), 8.41 (d, J = 7.2 Hz, 1H), 7.88 (s, 1H), | |
| | 7.73 (d, J = 8.4 Hz, 1H), 7.69 (d, J = 9.2 Hz, 1H), 7.50 (d, J = 8.8 Hz, | |
| | 1H), 7.39 (d, J = 8.0 Hz, 1H), 7.37-7.31 (m, 1H), 6.97 (t, J = 6.8 Hz, | |
| | 1H), 6.89 (d, J = 8.4 Hz, 1H), 6.61 (t, J = 8.0 Hz, 1H), 5.10-4.99 (m, | |
| | 1H), 4.51-4.34 (m, 3H), 4.26 (d, J = 16.4 Hz, 1H), 4.12 (s, 2H), 3.98 | |
| | (s, 2H), 3.60-3.38 (m, 2H), 3.28-3.09 (m, 5H), 3.05-2.83 (m, 3H), | |
| | 2.70-2.53 (m, 3H), 2.51 ( s, 3H), 2.47-2.30 (m, 3H), 2.15-2.03 (m, | |
| | 2H), 2.01-1.90 (m, 1H) | |
| 222 | ¹H NMR (400 MHz, DMSO-d₆) δ 11.00-10.92 (m, 1H), 9.94 (s, 1H), | B |
| | 8.79 (s, 1H), 8.66 (d, J = 8.4 Hz, 1H), 8.44 (dd, J = 6.0, 7.2 Hz, 1H), | |
| | 7.81 (s, 1H), 7.70 (d, J = 8.4 Hz, 1H), 7.52 (dd, J = 2.4, 10.0 Hz, 1H), | |
| | 7.46 (d, J = 8.4 Hz, 1H), 6.99-6.93 (m, 1H), 6.91 ( d, J = 7.2 Hz, 1H), | |
| | 6.84 (d, J = 8.4 Hz, 1H), 5.07-4.98 (m, 1H), 4.45-4.33 (m, 3H), 4.24 | |
| | (d, J = 16.8 Hz, 1H), 3.61-3.52 (m, 2H), 2.96-2.74 (m, 8H), 2.73- | |
| | 2.66 (m, 4H), 2.63-2.56 (m, 2H), 2.54 (s, 3H), 2.47 ( s, 3H), 2.00- | |
| | 1.86 (m, 3H), 1.70-1.54 (m, 2H) | |
| 223 | ¹H NMR (400 MHz, DMSO-d₆) δ 10.98 (s, 1H), 10.02-9.94 (m, 1H), | B |
| | 8.82 (s, 1H), 8.73 (d, J = 8.8 Hz, 1H), 8.45 (dd, J = 6.0, 7.6 Hz, 1H), | |
| | 7.82 (s, 1H), 7.72 (d, J = 8.4 Hz, 1H), 7.60-7.49 (m, 2H), 6.97 (dt, J = | |
| | 2.8, 7.6 Hz, 1H), 6.91 (d, J = 7.6 Hz, 1H), 6.82 (d, J = 8.6 Hz, 1H), 5.03 | |
| | (dd, J = 5.5, 13.2 Hz, 1H), 4.47-4.35 (m, 3H), 4.24 (d, J = 16.8 Hz, | |
| | 1H), 3.62-3.51 (m, 2H), 3.04 ( d, J = 10.0 Hz, 2H), 2.96-2.85 (m, | |
| | 1H), 2.83-2.73 (m, 2H), 2.66-2.55 (m, 3H), 2.54 (s, 3H), 2.49 ( s, | |
| | 3H), 2.47-2.33 (m, 3H), 2.00-1.85 (m, 3H), 1.77-1.55 (m, 6H) | |
| 224 | ¹H NMR (400 MHz, DMSO-d₆) δ 11.05 (s, 1H), 9.97 (s, 1H), 9.14 (d, | A |
| | J = 1.2 Hz, 1H), 8.82 (s, 1H), 8.69 (d, J = 8.8 Hz, 1H), 8.48 (dd, J = 1.2, | |
| | 4.8 Hz, 1H), 8.15 (s, 1H), 8.11 (s, 1H), 7.90 (d, J = 4.4 Hz, 1H), 7.78 (d, | |
| | J = 8.8 Hz, 1H), 7.48 (d, J = 8.8 Hz, 1H), 6.94-6.88 (m, 2H), 6.85 (d, J = | |
| | 8.4 Hz, 1H), 6.63 (d, J = 8.4 Hz, 1H), 5.30-5.22 (m, 1H), 4.59 (t, J = | |
| | 7.2 Hz, 1H), 4.44 (s, 2H), 3.82-3.74 (m, 1H), 3.59-3.53 (m, 2H), 2.85 | |
| | (s, 6H), 2.73 (s, 1H), 2.67-2.61 (m, 4H), 2.55-2.54 (m, 1H), 2.47 (s, | |
| | 3H), 2.26 (d, J = 7.2 Hz, 2H), 2.00-1.95 (m, 1H), 1.83 (d, J = 12.0 Hz, | |
| | 2H), 1.71-1.64 (m, 1H), 1.45 (d, J = 6.8 Hz, 6H), 1.34-1.25 (m, 2H) | |
| 225 | ¹H NMR (400 MHz, DMSO-d₆) δ 11.07 (s, 1H), 10.02 (s, 1H), 9.15 (d, | B |
| | J = 1.2 Hz, 1H), 8.85 (s, 1H), 8.71 (d, J = 8.4 Hz, 1H), 8.48 (dd, J = 1.6, | |
| | 4.8 Hz, 1H), 8.14-8.11 (m, 1H), 7.91 (d, J = 4.8 Hz, 1H), 7.79 (d, J = | |
| | 8.4 Hz, 1H), 7.50 (d, J = 8.8 Hz, 1H), 6.94 (d, J = 8.4 Hz, 1H), 6.91- | |
| | 6.85 (m, 2H), 6.70 (d, J = 7.6 Hz, 1H), 5.27 (dd, J = 5.2, 12.8 Hz, 1H), | |
| | 4.45 (s, 2H), 3.77-3.66 (m, 2H), 3.06 (d, J = 2.8 Hz, 7H), 2.95-2.80 | |
| | (m, 3H), 2.77-2.57 (m, 5H), 2.54 (s, 3H), 2.20-2.05 (m, 2H), 1.98 | |
| | (dd, J = 4.4, 10.4 Hz, 1H), 1.88-1.70 (m, 2H), 1.10-1.01 (m, 2H), | |
| | 0.93-0.85 (m, 2H) | |
| 226 | ¹H NMR (400 MHz, DMSO-d₆) δ 11.05 (s, 1H), 9.86 (s, 1H), 8.73 (s, | A |
| | 1H), 8.55 (d, J = 8.4 Hz, 1H), 8.17 (s, 1H), 7.50 (d, J = 8.8 Hz, 1H), | |
| | 7.45 (d, J = 8.8 Hz, 1H), 7.04 (s, 1H), 6.92 (d, J = 8.4 Hz, 1H), 6.88 (s, | |
| | 1H), 6.80 (d, J = 8.4 Hz, 1H), 6.69-6.57 (m, 1H), 5.26 (dd, J = 12.8, | |
| | 5.2 Hz, 1H), 4.69-4.50 (m, 1H), 4.43-4.30 (m, 2H), 3.93-3.82 (m, | |
| | 2H), 3.56 (d, J = 12.0 Hz, 2H), 2.93-2.77 (m, 7H), 2.71-2.52 (m, 8H), | |
| | 2.45 (s, 3H), 2.26 (d, J = 6.8 Hz, 2H), 2.01-1.94 (m, 1H), 1.90-1.78 | |
| | (m, 6H), 1.73-1.60 (m, 1H), 1.45 (d, J = 6.8 Hz, 6H), 1.34-1.23 (m, | |
| | 2H) | |
| 227 | ¹H NMR (400 MHz, DMSO-d₆) δ 11.06 (s, 1H), 9.97 (s, 1H), 9.13 (d, | B |
| | J = 1.2 Hz, 1H), 8.83 (s, 1H), 8.69 (d, J = 8.8 Hz, 1H), 8.47 (dd, J = 1.2, | |
| | 4.8 Hz, 1H), 8.11 (s, 1H), 7.90 (d, J = 4.8 Hz, 1H), 7.77 (d, J = 8.8 Hz, | |
| | 1H), 7.45 (d, J = 8.8 Hz, 1H), 6.91 (s, 2H), 6.84 (d, J = 8.4 Hz, 1H), | |
| | 6.63 (dd, J = 1.2, 8.4 Hz, 1H), 5.26 (dd, J = 5.2, 12.4 Hz, 1H), 4.64- | |
| | 4.54 (m, 1H), 4.43 (s, 2H), 3.62 (d, J = 10.4 Hz, 2H), 2.85 (s, 5H), | |
| | 2.74-2.57 (m, 9H), 2.46 (s, 3H), 2.00-1.87 (m, 3H), 1.61 (d, J = 9.2 Hz, | |
| | 2H), 1.45 (d, J = 6.8 Hz, 6H) | |
| 228 | ¹H NMR (400 MHz, DMSO-d₆) δ 11.07 (s, 1H), 9.94 (s, 1H), 8.81 (s, | B |
| | 1H), 8.67 (d, J = 8.4 Hz, 1H), 8.48-8.41 (m, 1H), 7.82 (s, 1H), 7.71 (d, | |
| | J = 8.4 Hz, 1H), 7.53 (dd, J1 = 2.4, J2 = 10.0 Hz, 1H), 7.47 (d, J = 8.4 | |
| | Hz, 1H), 7.01-6.89 (m, 3H), 6.85 (d, J = 8.4 Hz, 1H), 6.65 (dd, J1 = | |
| | 2.0, J2 = 8.8 Hz, 1H), 5.32-5.22 (m, 1H), 4.65-4.55 (m, 1H), 4.39 (s, | |
| | 2H), 3.64 (d, J = 11.2 Hz, 2H), 2.91-2.81 (m, 5H), 2.74-2.58 (m, 8H), | |
| | 2.47 (s, 3H), 2.41-2.34 (m, 1H), 2.03-1.96 (m, 1H), 1.92 (d, J = 11.2 | |
| | Hz, 2H), 1.71-1.55 (m, 2H), 1.47 (s, 3H), 1.45 (s, 3H) | |
| 229 | ¹H NMR (400 MHz, DMSO-d₆) δ 10.63 (s, 1H), 9.94 (s, 1H), 8.79 (s, | A |
| | 1H), 8.66 (d, J = 8.4 Hz, 1H), 8.47-8.41 (m, 1H), 8.12 (s, 1H), 7.82 (s, | |
| | 1H), 7.71 (d, J = 8.4 Hz, 1H), 7.54-7.46 (m, 3H), 6.96 (dt, J = 2.4, 7.6 | |
| | Hz, 1H), 6.84 (d, J = 8.8 Hz, 1H), 6.46 (s, 1H), 4.45-4.34 (m, 4H), | |
| | 3.76 ( t, J = 6.0 Hz, 2H), 2.88-2.78 (m, 8H), 2.55 (s, 4H), 2.46 (s, 3H), | |
| | 2.27 (d, J = 6.4 Hz, 2H), 1.85 (d, J = 12.4 Hz, 3H), 1.36-1.24 (m, 2H) | |
| 230 | ¹H NMR (400 MHz, DMSO-d₆) δ 11.06 (s, 1H), 9.95 (s, 1H), 8.81 (s, | A |
| | 1H), 8.67 (d, J = 8.4 Hz, 1H), 8.42 (d, J = 7.2 Hz, 1H), 7.91-7.81 (m, | |
| | 2H), 7.71 (d, J = 8.4 Hz, 1H), 7.47 (d, J = 8.4 Hz, 1H), 6.97 (dd, J = 2.0, | |
| | 7.2 Hz, 1H), 6.92 (d, J = 8.4 Hz, 1H), 6.89 (d, J = 1.6 Hz, 1H), 6.84 (d, | |
| | J = 8.4 Hz, 1H), 6.63 (dd, J = 1.2, 8.8 Hz, 1H), 5.32-5.21 (m, 1H), 4.59 | |
| | (m, 1H), 4.39 (s, 2H), 3.56 (d, J = 12.0 Hz, 2H), 3.33 (s, 4H), 2.85 (s, | |
| | 4H), 2.69-2.59 (m, 4H), 2.55-2.53 (m, 2H), 2.46 (s, 3H), 2.26 (d, J = | |
| | 6.8 Hz, 2H), 2.02-1.94 (m, 1H), 1.83 (d, J = 12.0 Hz, 2H), 1.67 (s, | |
| | 1H), 1.45 (d, J = 6.8 Hz, 6H), 1.34-1.23 (m, 2H) | |
| 231 | ¹H NMR (400 MHz, DMSO-d₆) δ 11.05 (s, 1H), 9.90 (s, 1H), 8.99 (s, | A |
| | 1H), 8.73 (s, 1H), 8.54 (d, J = 8.4 Hz, 1H), 7.88 (s, 1H), 7.67 (s, 1H), | |
| | 7.57 (d, J = 8.4 Hz, 1H), 7.47 (d, J = 8.8 Hz, 1H), 6.95-6.80 (m, 3H), | |
| | 6.63 (dd, J = 2.0, 8.8 Hz, 1H), 5.26 (dd, J = 5.2, 12.8 Hz, 1H), 4.66- | |
| | 4.52 (m, 1H), 4.38-4.23 (m, 1H), 4.22-4.06 (m, 1H), 3.63-3.51 (m, | |
| | 2H), 2.93-2.79 (m, 5H), 2.73-2.53 (m, 8H), 2.46 (s, 3H), 2.34-2.25 | |
| | (m, 2H), 2.15 (s, 3H), 2.04-1.92 (m, 1H), 1.83 ( d, J = 11.2 Hz, 2H), | |
| | 1.74-1.62 (m, 1H), 1.45 (d, J = 6.8 Hz, 6H), 1.36-1.21 (m, 2H) | |
| 232 | ¹H NMR (400 MHz, DMSO-d₆) δ 11.06 (s, 1H), 9.96 (s, 1H), 8.80 (s, | A |
| | 1H), 8.68 (d, J = 8.4 Hz, 1H), 8.56 (d, J = 7.2 Hz, 1H), 8.44 (s, 1H), | |
| | 8.16-8.12 (m, 1H), 7.76 (d, J = 8.4 Hz, 1H), 7.48 (d, J = 8.4 Hz, 1H), | |
| | 7.20 (dd, J = 1.6, 7.2 Hz, 1H), 6.96-6.84 (m, 3H), 6.60 (dd, J = 1.6, 8.8 | |
| | Hz, 1H), 5.28 (dd, J = 5.2, 12.8 Hz, 1H), 4.60 (td, J = 6.8, 13.6 Hz, 1H), | |
| | 4.40 (s, 2H), 3.56 (d, J = 12.0 Hz, 2H), 2.92-2.80 (m, 5H), 2.72-2.56 | |
| | (m, 8H), 2.48 (s, 3H), 2.28 (d, J = 6.8 Hz, 2H), 2.00-1.92 (m, 1H), 1.88- | |
| | 1.80 (m, 2H), 1.72-1.60 (m, 1H), 1.44 (d, J = 6.8 Hz, 6H), 1.36-1.20 | |
| | (m, 2H) | |
| 233 | ¹H NMR (400 MHz, DMSO-d₆) δ 11.05 (s, 1H), 9.86 (s, 1H), 8.73 (s, | B |
| | 1H), 8.55 (d, J = 8.4 Hz, 1H), 8.16 (s, 1H), 7.50 (d, J = 8.8 Hz, 1H), | |
| | 7.45 (d, J = 8.8 Hz, 1H), 7.04 (s, 1H), 6.92 (d, J = 8.4 Hz, 1H), 6.88 (s, | |
| | 1H), 6.80 (d, J = 8.4 Hz, 1H), 6.69-6.57 (m, 1H), 5.25 (dd, J = 12.8, | |
| | 5.2 Hz, 1H), 4.37 (s, 1H), 3.87 (s, 2H), 3.61 (d, J = 12.0 Hz, 2H), 2.87- | |
| | 2.80 (m, 7H), 2.69-2.63 (m, 8H), 2.45 (s, 3H), 2.40-2.36 (m, 1H), | |
| | 1.93-1.86 (m, 7H), 1.65-1.62 (m, 1H), 1.05-1.04 (m, 2H), 0.89-0.87 | |
| | (m, 2H) | |
| 234 | ¹H NMR (400 MHz, DMSO-d₆) δ 11.08 (s, 1H), 9.98 (s, 1H), 9.14 (s, | B |
| | 1H), 8.84 (s, 1H), 8.70 (d, J = 8.4 Hz, 1H), 8.49 (d, J = 4.8 Hz, 1H), | |
| | 8.12 (s, 1H), 7.91 (d, J = 4.6 Hz, 1H), 7.78 (d, J = 8.6 Hz, 1H), 7.48 (br | |
| | d, J = 8.6 Hz, 1H), 7.03-6.80 (m, 4H), 5.70-5.54 (m, 1H), 5.30 ( dd, J = | |
| | 4.4, 12.4 Hz, 1H), 4.45 (s, 2H), 3.16 ( d, J = 9.2 Hz, 2H), 2.87 ( s, | |
| | 5H), 2.72 ( s, 5H), 2.61 ( d, J = 18.0 Hz, 2H), 2.48-2.48 (m, 3H), 2.44- | |
| | 2.36 (m, 2H), 2.05-1.94 (m, 3H), 1.67-1.54 (m, 2H), 1.49 ( dd, J = | |
| | 6.8, 10.4 Hz, 6H) | |
| 235 | ¹H NMR (400 MHz, DMSO-d₆) δ 11.09 (s, 1H), 10.00-9.87 (m, 1H), | A |
| | 9.03-8.97 (m, 1H), 8.81-8.72 (m, 1H), 8.57 (d, J = 4.4 Hz, 1H), 7.89 | |
| | (s, 1H), 7.68 (s, 1H), 7.58 (d, J = 8.0 Hz, 1H), 7.55-7.43 (m, 1H), | |
| | 7.05-6.78 (m, 4H), 5.69-5.53 (m, 1H), 5.36-5.24 (m, 1H), 4.39 (d, J = 3.2 | |
| | Hz, 1H), 4.22-4.08 (m, 1H), 3.76-3.62 (m, 1H), 3.29-3.03 (m, 4H), | |
| | 2.96-2.82 (m, 3H), 2.80-2.68 (m, 4H), 2.65-2.56 (m, 2H), 2.55- | |
| | 2.52 (m, 2H), 2.49-2.46 (m, 3H), 2.32-2.24 (m, 1H), 2.16 (s, 3H), | |
| | 2.03-1.94 (m, 2H), 1.91-1.79 (m, 1H), 1.66-1.55 (m, 1H), 1.50 (s, | |
| | 6H) | |
| 236 | 1H NMR (400 MHz, DMSO-d6) 8 11.06 (s, 1H), 9.91-9.82 (m, 1H), | A |
| | 8.81-8.71 (m, 1H), 8.63 (d, J = 8.8 Hz, 1H), 7.57-7.38 (m, 1H), 7.18- | |
| | 7.07 (m, 1H), 6.98-6.82 (m, 2H), 6.70-6.57 (m, 2H), 5.33-5.20 (m, | |
| | 1H), 4.68-4.50 (m, 1H), 4.44-4.28 (m, 2H), 3.95-3.79 (m, 2H), 3.71- | |
| | 3.51 (m, 2H), 3.17-2.92 (m, 4H), 2.90-2.79 (m, 3H), 2.77-2.53 (m, | |
| | 9H), 2.46 (s, 3H), 2.29 (s, 3H), 2.02-1.79 (m, 8H), 1.45 (d, J = 6.8 Hz, | |
| | 6H), 1.37-1.21 (m, 3H) | |
| 237 | ¹H NMR (400 MHz, DMSO-d₆) δ 11.00-10.96 (m, 1H), 10.00-9.92 | A |
| | (m, 1H), 8.80 (s, 1H), 8.68 (d, J = 8.8 Hz, 1H), 8.56 (d, J = 7.2 Hz, 1H), | |
| | 8.48-8.40 (m, 1H), 8.16 (s, 1H), 7.76 (d, J = 8.4 Hz, 1H), 7.64 (d, J = | |
| | 8.0 Hz, 1H), 7.48 (d, J = 8.8 Hz, 1H), 7.28 (d, J = 8.4 Hz, 1H), 7.20- | |
| | 7.16 (m, 1H), 6.88 (d, J = 8.8 Hz, 1H), 5.16-5.02 (m, 1H), 4.48-4.36 | |
| | (m, 3H), 4.32-4.20 (m, 1H), 3.52-3.44 (m, 2H), 2.88 (s, 4H), 2.84- | |
| | 2.76 (m, 2H), 2.72-2.68 (m, 4H), 2.48 (s, 3H), 2.44 (d, J = 4.4 Hz, 4H), | |
| | 2.04-1.96 (m, 1H), 1.92 (d, J = 12.10Hz, 2H), 1.76-1.64 (m, 2H) | |
| 238 | ¹H NMR (400 MHz, DMSO-d₆) δ 10.55 (s, 1H), 9.96 (s, 1H), 8.81 (s, | A |
| | 1H), 8.67 (d, J = 8.8 Hz, 1H), 8.63 (d, J = 2.4 Hz, 1H), 8.44 (dd, J = 6.0, | |
| | 7.2 Hz, 1H), 8.13 (s, 1H), 8.00 (d, J = 2.4 Hz, 1H), 7.84-7.80 (m, 2H), | |
| | 7.71 (d, J = 8.4 Hz, 1H), 7.58 (dd, J = 2.4, 9.2 Hz, 1H), 7.52 (dd, J = | |
| | 2.4, 10.0 Hz, 1H), 7.48 (d, J = 8.4 Hz, 1H), 7.16 (d, J = 2.4 Hz, 1H), | |
| | 6.96 (m, 1H), 6.86 (d, J = 8.8 Hz, 1H), 4.39 (s, 2H), 3.95-3.90 (m, 3H), | |
| | 3.88 (s, 1H), 2.90 (s, 4H), 2.83 (d, J = 12.4 Hz, 2H), 2.78 (t, J = 6.4 Hz, | |
| | 3H), 2.55-2.51 (m, 3H), 2.48 (s, 2H), 2.47 (s, 3H), 1.87 (d, J = 11.6 | |
| | Hz, 3H), 1.37-1.22 (m, 2H) | |
| 239 | ¹H NMR (400 MHz, DMSO-d₆) δ 10.52 (s, 1H), 9.96 (s, 1H), 8.80 (s, | A |
| | 1H), 8.68 (d, J = 8.4 Hz, 1H), 8.64 (d, J = 2.4 Hz, 1H), 8.44 (dd, J = 6.0, | |
| | 7.6 Hz, 1H), 7.96 (d, J = 2.4 Hz, 1H), 7.80 (s, 1H), 7.72 (d, J = 8.8 Hz, | |
| | 1H), 7.52 (dd, J = 2.8, 10.0 Hz, 1H), 7.48-7.44 (m, 2H), 7.00-6.92 | |
| | (m, 2H), 6.84 (d, J = 8.8 Hz, 1H), 4.36 (s, 2H), 3.96-3.84 (m, 4H), 2.84 | |
| | (s, 4H), 2.80-2.76 (m, 4H), 2.64 (s, 3H), 2.52 (d, J = 6.8 Hz, 4H), 2.48 | |
| | (s, 3H), 2.28-2.20 (m, 2H), 1.84 (d, J = 12.4 Hz, 2H), 1.80-1.72 (m, | |
| | 1H), 1.32-1.20 (m, 2H) | |
| 240 | ¹H NMR (400 MHz, DMSO-d₆) δ 11.00 (s, 1H), 9.93 (s, 1H), 8.79 (s, | C |
| | 1H), 8.66 (d, J = 8.8 Hz, 1H), 8.51-8.33 (m, 1H), 7.81 (s, 1H), 7.70 (d, | |
| | J = 8.4 Hz, 1H), 7.58-7.48 (m, 3H), 7.45 (d, J = 8.8 Hz, 1H), 6.96 (dt, J = | |
| | 7.6, 2.4 Hz, 1H), 6.84 (d, J = 8.4 Hz, 1H), 5.11 (dd, J = 13.2, 4.8 Hz, | |
| | 1H), 4.55 (d, J = 17.6 Hz, 1H), 4.46-4.26 (m, 3H), 3.16-3.04 (m, 4H), | |
| | 2.99-2.84 (m, 2H), 2.73-2.62 (m, 2H), 2.60-2.58 (m, 1H), 2.54 (s, | |
| | 2H), 2.46 (s, 3H), 2.42-2.35 (s, 2H), 2.06-1.96 (m, 1H), 1.95-1.85 | |
| | (m, 2H), 1.81 (d, J = 5.2 Hz, 4H), 1.73-1.62 (m, 2H) | |
| 241 | ¹H NMR (400 MHz, DMSO-d₆) δ 11.00 (s, 1H), 9.84 (s, 1H), 8.75 (s, | C |
| | 1H), 8.55 (d, J = 8.4 Hz, 1H), 8.48-8.36 (m, 1H), 8.01 (d, J = 2.8 Hz, | |
| | 1H), 7.80 (s, 1H), 7.68 (d, J = 8.4 Hz, 1H), 7.57-7.49 (m, 3H), 7.45 | |
| | (dd, J = 8.8, 2.8 Hz, 1H), 7.03-6.87 (m, 2H), 5.11 (dd, J = 13.2, 4.8 Hz, | |
| | 1H), 4.61-4.49 (m, 1H), 4.46-4.30 (m, 3H), 3.79-3.58 (m, 2H), 3.06 | |
| | (d, J = 10.4 Hz, 2H), 2.99-2.84 (m, 2H), 2.73-2.65 (m, 1H), 2.65- | |
| | 2.54 (m, 2H), 2.46-2.34 (m, 4H), 2.05-1.96 (m, 1H), 1.94-1.85 (m, | |
| | 2H), 1.83-1.70 (m, 4H), 1.69-1.53 (m, 2H) | |
| 242 | ¹H NMR (400 MHz, DMSO-d₆) δ 10.56 (s, 1H), 9.96 (s, 1H), 9.34 (s, | A |
| | 1H), 8.83-8.77 (m, 1H), 8.69-8.64 (m, 1H), 8.62-8.54 (m, 1H), 8.47- | |
| | 8.42 (m, 1H), 8.14-7.92 (m, 2H), 7.84-7.81 (m, 1H), 7.70 (d, J = 8.4 | |
| | Hz, 1H), 7.56-7.46 (m, 2H), 7.02-6.95 (m, 1H), 6.90-6.83 (m, 1H), | |
| | 4.38 (s, 2H), 3.96 (d, J = 4.4 Hz, 1H), 3.77-3.71 (m, 1H), 3.10-2.88 | |
| | (m, 6H), 2.85-2.59 (m, 7H), 2.47 (s, 3H) | |
| 243 | ¹H NMR (400 MHz, DMSO-d₆) δ 10.62 (s, 1H), 9.94 (s, 1H), 8.80 (s, | A |
| | 1H), 8.66 (d, J = 8.8 Hz, 1H), 8.44 (t, J = 6.0 Hz, 1H), 7.82 (d, J = 1.2 | |
| | Hz, 1H), 7.71 (d, J = 8.4 Hz, 1H), 7.51 (s, 5H), 7.28 (d, J = 9.6 Hz, 1H), | |
| | 7.00-6.92 (m, 1H), 6.84 (d, J = 8.0 Hz, 1H), 4.38 (s, 2H), 3.78 (t, J = | |
| | 6.0 Hz, 2H), 3.56 (d, J = 9.6 Hz, 3H), 2.84 (s, 6H), 2.63-2.53 (m, 5H), | |
| | 2.46 (s, 3H), 2.27 (d, J = 6.4 Hz, 2H), 1.91-1.79 (m, 2H), 1.69 (s, 1H), | |
| | 1.29 (d, J = 11.2 Hz, 2H) | |
| 244 | ¹H NMR (400 MHz, DMSO-d₆) δ 10.63 (s, 1H), 9.93 (s, 1H), 8.80 (s, | B |
| | 1H), 8.66 (d, J = 8.4 Hz, 1H), 8.48-8.39 (m, 1H), 8.17 (s, 1H), 7.82 (s, | |
| | 1H), 7.70 (d, J = 8.4 Hz, 1H), 7.66 (s, 1H), 7.52 (dd, J = 2.4, 9.6 Hz, | |
| | 1H), 7.46 (d, J = 8.8 Hz, 1H), 7.40 (s, 1H), 6.96 (dt, J = 2.4, 7.2 Hz, | |
| | 1H), 6.84 (d, J = 8.4 Hz, 1H), 6.38 (s, 1H), 4.38 (s, 3H), 3.74 (s, 2H), | |
| | 2.85 (s, 6H), 2.71 (s, 8H), 2.47 (s, 3H), 2.25 (s, 3H), 1.93 (d, J = 9.2 Hz, | |
| | 2H), 1.64 (d, J = 8.4 Hz, 2H) | |
| 245 | ¹H NMR (400 MHz, DMSO-d₆) δ 11.04 (s, 1H), 9.92 (s, 1H), 8.80 (s, | C |
| | 1H), 8.68 (d, J = 8.4 Hz, 1H), 8.48-8.40 (m, 1H), 7.80 (s, 1H), 7.72 (d, | |
| | J = 8.8 Hz, 1H), 7.52 (dd, J = 2.8, 10.0 Hz, 1H), 7.44 (d, J = 8.8 Hz, | |
| | 1H), 7.00-6.88 (m, 3H), 6.84 (d, J = 8.4 Hz, 1H), 6.68-6.60 (m, 1H), | |
| | 5.28 (dd, J = 5.2, 12.8 Hz, 1H), 4.60 (m, J = 6.8 Hz, 1H), 4.36 (s, 2H), | |
| | 3.16-3.02 (m, 6H), 2.96-2.84 (m, 1H), 2.76-2.60 (m, 8H), 2.48 (s, | |
| | 3H), 2.40-2.32 (m, 1H), 2.04-1.88 (m, 3H), 1.68-1.56 (m, 2H), 1.44 | |
| | (d, J = 6.8 Hz, 6H) | |
| 246 | ¹H NMR (400 MHz, DMSO-d₆) δ 11.06-10.94 (m, 1H), 9.91-9.80 | B |
| | (m, 1H), 8.75 (s, 1H), 8.62 (d, J = 8.4 Hz, 1H), 7.69-7.61 (m, 1H), 7.50 | |
| | (d, J = 8.8 Hz, 1H), 7.33 (s, 1H), 7.04 (s, 1H), 6.63 (s, 1H), 5.14-5.04 | |
| | (m, 1H), 4.46-4.34 (m, 3H), 4.26 (d, J = 17.6 Hz, 1H), 3.93-3.77 (m, | |
| | 2H), 3.54 (d, J = 5.2 Hz, 2H), 3.06-2.95 (m, 4H), 2.83-2.72 (m, 4H), | |
| | 2.69-2.62 (m, 4H), 2.46 (s, 6H), 2.35-2.17 (m, 4H), 2.03-1.90 (m, | |
| | 3H), 1.89-1.81 (m, 4H), 1.73-1.61 (m, 2H) | |
| 247 | ¹H NMR (400 MHz, DMSO-d₆) δ 11.06 (s, 1H), 9.94 (s, 1H), 8.79 (s, | A |
| | 1H), 8.66 (d, J = 8.4 Hz, 1H), 8.44 (dd, J = 6.0, 7.6 Hz, 1H), 7.81 (s, | |
| | 1H), 7.70 (d, J = 8.4 Hz, 1H), 7.52 (dd, J = 2.4, 10.0 Hz, 1H), 7.46 (d, J = | |
| | 8.4 Hz, 1H), 6.96 (dt, J = 2.4, 7.6 Hz, 1H), 6.91 (d, J = 8.4 Hz, 1H), | |
| | 6.88-6.80 (m, 2H), 6.66 (dd, J = 2.0, 8.8 Hz, 1H), 5.25 ( dd, J = 5.2, | |
| | 12.4 Hz, 1H), 4.38 (s, 2H), 3.55 ( d, J = 11.4 Hz, 2H), 2.96-2.78 (m, | |
| | 6H), 2.73-2.60 (m, 4H), 2.57 ( d, J = 3.6 Hz, 4H), 2.46 (s, 3H), 2.26 | |
| | (d, J = 7.2 Hz, 2H), 2.03-1.92 (m, 1H), 1.84 ( d, J = 12.0 Hz, 2H), | |
| | 1.67 ( d, J = 3.6 Hz, 1H), 1.39-1.20 (m, 2H), 1.10-1.00 (m, 2H), | |
| | 0.91-0.80 (m, 2H) | |
| 248 | ¹H NMR (400 MHz, DMSO-d₆) δ 11.08 (s, 1H), 10.35 (s, 1H), 8.93 (s, | A |
| | 1H), 8.72 (d, J = 8.4 Hz, 1H), 8.48 (dd, J = 6.0, 7.2 Hz, 1H), 8.14 (s, | |
| | 1H), 8.01 (d, J = 9.2 Hz, 1H), 7.85 (s, 1H), 7.78 (d, J = 8.4 Hz, 1H), | |
| | 7.53 (dd, J = 2.4, 10.0 Hz, 1H), 7.32 (d, J = 8.8 Hz, 1H), 6.99-6.86 (m, | |
| | 3H), 6.63 (dd, J = 1.6, 8.4 Hz, 1H), 5.32-5.21 (m, 1H), 4.64-4.54 (m, | |
| | 1H), 4.43 (s, 2H), 3.56 (d, J = 11.6 Hz, 2H), 3.35 (s, 3H), 2.89 (s, 5H), | |
| | 2.69-2.57 (m, 5H), 2.34-2.24 (m, 2H), 2.02-1.94 (m, 1H), 1.83 (d, | |
| | J = 11.6 Hz, 2H), 1.72-1.60 (m, 1H), 1.45 (d, J = 6.8 Hz, 6H), | |
| | 1.34-1.20 (m, 2H) | |
| 249 | ¹H NMR (400 MHz, DMSO-d₆) δ 11.07 (s, 1H), 9.95 (s, 1H), 8.80 (s, | A |
| | 1H), 8.66 (d, J = 8.4 Hz, 1H), 8.52 (dd, J = 2.4, 4.4 Hz, 1H), 7.90 (s, | |
| | 1H), 7.78-7.70 (m, 2H), 7.47 (d, J = 8.6 Hz, 1H), 7.38 (ddd, J = 2.4, | |
| | 7.6, 9.9 Hz, 1H), 6.96-6.81 (m, 3H), 6.63 (dd, J = 1.6, 8.8 Hz, 1H), | |
| | 5.27 (dd, J = 5.2, 12.8 Hz, 1H), 4.65-4.52 (m, 1H), 4.39 (s, 2H), 3.61- | |
| | 3.52 (m, 2H), 3.34 (s, 6H), 2.92-2.80 (m, 5H), 2.67-2.61 (m, 3H), | |
| | 2.46 (s, 3H), 2.26 (d, J = 6.4 Hz, 2H), 2.01-1.94 (m, 1H), 1.83 (d, J = | |
| | 11.2 Hz, 2H), 1.73-1.62 (m, 1H), 1.45 (d, J = 6.4 Hz, 6H), 1.35-1.23 | |
| | (m, 2H) | |
| 250 | ¹H NMR (400 MHz, DMSO-d₆) δ 11.08 (s, 1H), 10.22 (s, 1H), 8.91 (s, | A |
| | 1H), 8.59 (d, J = 8.4 Hz, 1H), 8.50 (dd, J = 6.0, 7.2 Hz, 1H), 7.87-7.79 | |
| | (m, 2H), 7.70 (d, J = 9.2 Hz, 1H), 7.53 (dd, J = 2.6, 10.0 Hz, 1H), 7.34 | |
| | (d, J = 9.2 Hz, 1H), 6.99-6.87 (m, 3H), 6.66-6.60 (m, 1H), 5.27 (dd, J = | |
| | 5.2, 12.8 Hz, 1H), 4.59 (td, J = 6.8, 13.8 Hz, 1H), 4.43 (s, 2H), 3.56 | |
| | (d, J = 11.6 Hz, 2H), 3.14 (s, 4H), 2.92-2.84 (m, 1H), 2.66-2.53 (m, | |
| | 8H), 2.26 (d, J = 6.8 Hz, 2H), 2.02-1.94 (m, 1H), 1.82 (d, J = 11.2 Hz, | |
| | 2H), 1.69 (d, J = 10.4 Hz, 1H), 1.45 (d, J = 6.8 Hz, 6H), 1.34-1.23 (m, | |
| | 2H) | |
| 251 | ¹H NMR (400 MHz, DMSO-d₆) δ 10.65 (s, 1H), 9.94 (s, 1H), 8.81 (s, | B |
| | 1H), 8.67 (d, J = 8.8 Hz, 1H), 8.44 (d, J = 6.0 Hz, 1H), 8.14 (s, 1H), | |
| | 7.82 (s, 1H), 7.71 (d, J = 8.0 Hz, 1H), 7.51 (s, 2H), 7.46 (d, J = 8.8 Hz, | |
| | 1H), 6.99-6.93 (m, 1H), 6.84 (d, J = 8.0 Hz, 1H), 6.49 (s, 1H), 4.47 (d, | |
| | J = 6.0 Hz, 2H), 4.39 (s, 2H), 3.76 (d, J = 5.6 Hz, 2H), 2.84 (d, J = 0.8 | |
| | Hz, 8H), 2.71 (s, 5H), 2.47 (s, 3H), 1.98-1.90 (m, 2H), 1.68-1.55 (m, | |
| | 2H) | |
| 252 | ¹H NMR (400 MHz, DMSO-d₆) δ 11.06 (s, 1H), 9.94 (s, 1H), 8.78 (s, | A |
| | 1H), 8.67 (d, J = 8.4 Hz, 1H), 8.18 (s, 1H), 7.76 (s, 1H), 7.70 (d, J = 8.4 | |
| | Hz, 1H), 7.56 (d, J = 9.2 Hz, 1H), 7.46 (d, J = 8.8 Hz, 1H), 7.15 (dd, J = | |
| | 1.6, 9.2 Hz, 1H), 7.00-6.79 (m, 3H), 6.63 (dd, J = 1.6, 8.8 Hz, 1H), | |
| | 5.27 (dd, J = 5.6, 12.4 Hz, 1H), 4.59 (td, J = 7.2, 13.6 Hz, 1H), 4.44- | |
| | 4.26 (m, 2H), 3.56 (d, J = 11.6 Hz, 2H), 3.34 (s, 6H), 2.85 (br s, 4H), | |
| | 2.66-2.61 (m, 3H), 2.46 (s, 3H), 2.32-2.21 (m, 5H), 2.04-1.92 (m, | |
| | 1H), 1.83 (d, J = 12.8 Hz, 2H), 1.72-1.62 (m, 1H), 1.45 (d, J = 6.8 Hz, | |
| | 6H), 1.35-1.21 (m, 2H) | |
| 253 | ¹H NMR (400 MHz, DMSO-d₆) δ 11.08 (s, 1H), 9.93 (s, 1H), 8.79 (s, | C |
| | 1H), 8.66 (d, J = 8.4 Hz, 1H), 8.44 (dd, J = 6.0, 7.6 Hz, 1H), 7.81 (s, | |
| | 1H), 7.70 (d, J = 8.4 Hz, 1H), 7.51 (dd, J = 2.4, 10.0 Hz, 1H), 7.44 (d, | |
| | J = 8.8 Hz, 1H), 7.19 (s, 1H), 7.01-6.93 (m, 2H), 6.90 (d, J = 9.2 Hz, | |
| | 1H), 6.83 (d, J = 8.8 Hz, 1H), 5.31 (dd, J = 5.2, 12.4 Hz, 1H), 4.61 (t, | |
| | J = 7.2 Hz, 1H), 4.38 (s, 2H), 3.08-3.01 (m, 4H), 2.92-2.83 (m, 1H), | |
| | 2.71-2.59 (m, 4H), 2.45 (s, 3H), 2.35-2.24 (m, 3H), 2.01-1.94 (m, | |
| | 1H), 1.90-1.60 (m, 9H), 1.45 (d, J = 6.8 Hz, 6H) | |
| 254 | ¹H NMR (400 MHz, DMSO-d₆) δ 11.05 (s, 1H), 10.00 (s, 1H), 8.85- | A |
| | 8.67 (m, 2H), 8.42 (dd, J = 6.0, 7.2 Hz, 1H), 8.14 (s, 1H), 7.82 (s, 1H), | |
| | 7.68 (d, J = 8.4 Hz, 1H), 7.63-7.47 (m, 2H), 7.07-6.85 (m, 4H), 6.63 | |
| | (dd, J = 1.2, 8.8 Hz, 1H), 5.26 (dd, J = 5.6, 12.6 Hz, 1H), 5.14-4.95 (m, | |
| | 1H), 4.69-4.53 (m, 3H), 4.39 (s, 2H), 3.67-3.48 (m, 3H), 2.97-2.76 | |
| | (m, 6H), 2.72-2.57 (m, 6H), 2.27 (br d, J = 7.2 Hz, 2H), 2.04-1.95 (m, | |
| | 1H), 1.83 (d, J = 11.8 Hz, 2H), 1.72-1.63 (m, 1H), 1.45 (d, J = 6.8 Hz, | |
| | 6H), 1.36-1.23 (m, 2H) | |
| 255 | ¹H NMR (400 MHz, DMSO-d₆) δ 11.06 (s, 1H), 9.88 (s, 1H), 8.77 (m, | A |
| | 1H), 8.72 (d, J = 8.0 Hz, 1H), 7.80 (s, 1H), 7.53 (dd, J = 8.4, 14.2 Hz, | |
| | 2H), 7.31-7.22 (m, 2H), 6.95-6.87 (m, 2H), 6.84 (d, J = 8.4 Hz, 1H), | |
| | 6.63 ( d, J = 8.8 Hz, 1H), 6.31 (d, J = 7.2 Hz, 1H), 5.26 ( dd, J = 5.2, | |
| | 12.7 Hz, 1H), 4.59 (td, J = 7.2, 13.6 Hz, 1H), 4.23 (s, 2H), 3.76 (s, 3H), | |
| | 3.56 ( d, J = 11.6 Hz, 2H), 2.92-2.80 (m, 5H), 2.74-2.52 (m, 8H), | |
| | 2.45 (s, 3H), 2.26 ( d, J = 6.8 Hz, 2H), 2.02-1.94 (m, 1H), 1.83 ( d, J = | |
| | 11.6 Hz, 2H), 1.74-1.61 (m, 1H), 1.45 (d, J = 6.8 Hz, 6H), 1.34-1.23 | |
| | (m, 2H) | |
| 256 | ¹H NMR (400 MHz, DMSO-d₆) δ 11.06 (s, 1H), 9.97 (s, 1H), 8.79 (s, | A |
| | 1H), 8.70-8.65 (m, 2H), 8.02 (s, 1H), 7.87 (d, J = 9.6 Hz, 1H), 7.80 (d, | |
| | J = 8.4 Hz, 1H), 7.56-7.46 (m, 2H), 6.94-6.85 (m, 3H), 6.64 (dd, J = | |
| | 1.6, 8.7 Hz, 1H), 5.27 (dd, J = 5.2, 12.8 Hz, 1H), 4.59 (td, J = 6.8, 13.9 | |
| | Hz, 1H), 4.40 (s, 2H), 3.57 (d, J = 11.2 Hz, 2H), 2.97-2.83 (m, 6H), | |
| | 2.69-2.58 (m, 6H), 2.50-2.49 (m, 3H), 2.47 (s, 3H), 2.01-1.95 (m, | |
| | 1H), 1.83 (d, J = 11.2 Hz, 2H), 1.73 (s, 1H), 1.45 (d, J = 6.8 Hz, 6H), | |
| | 1.35-1.26 (m, 2H) | |
| 257 | ¹H NMR (400 MHz, DMSO-d₆) δ 11.07 (s, 1H), 9.95 (s, 1H), 8.79 (s, | A |
| | 1H), 8.67 (d, J = 8.4 Hz, 1H), 8.49 (s, 1H), 7.90 (s, 1H), 7.74 (dd, J = | |
| | 9.2, 13.6 Hz, 2H), 7.48 (d, J = 8.8 Hz, 1H), 7.35 (dd, J = 1.6, 9.6 Hz, | |
| | 1H), 6.98-6.80 (m, 3H), 6.64 (d, J = 8.4 Hz, 1H), 5.27 (dd, J = 5.2, | |
| | 12.8 Hz, 1H), 4.70-4.50 (m, 1H), 4.38 (s, 2H), 3.57 (d, J = 12.0 Hz, | |
| | 2H), 2.93-2.77 (m, 5H), 2.63 (s, 4H), 2.55 (s, 4H), 2.47 (s, 3H), 2.27 | |
| | (d, J = 7.2 Hz, 2H), 2.04-1.93 (m, 1H), 1.88-1.78 (m, 2H), 1.75-1.60 | |
| | (m, 1H), 1.46 (s, 3H), 1.45 (s, 3H), 1.36-1.22 (m, 2H) | |
| 258 | ¹H NMR (400 MHz, DMSO-d₆) δ 11.06 (s, 1H), 9.99 (s, 1H), 8.78 (s, | A |
| | 1H), 8.62 (d, J = 8.4 Hz, 1H), 8.30 (d, J = 4.8 Hz, 1H), 7.73 (d, J = 8.4 | |
| | Hz, 1H), 7.57 (d, J = 3.6 Hz, 1H), 7.47 (d, J = 8.4 Hz, 1H), 7.25 (d, J = | |
| | 4.8 Hz, 1H), 6.96-6.83 (m, 3H), 6.63 (dd, J1 = 1.6, J2 = 8.4 Hz, 1H), | |
| | 6.47 (d, J = 3.6 Hz, 1H), 5.26 (dd, J1 = 5.2, J2 =12.4 Hz, 1H), 4.64- | |
| | 4.53 (m, 1H), 4.45 (s, 2H), 3.86 (s, 3H), 3.56 (d, J = 11.6 Hz, 2H), 2.90- | |
| | 2.79 (m, 5H), 2.72-2.56 (m, 6H), 2.54 (s, 2H), 2.46 (s, 3H), 2.26 (d, J = | |
| | 6.8 Hz, 2H), 2.01-1.94 (m, 1H), 1.83 (d, J = 11.6 Hz, 2H), 1.73- | |
| | 1.58 (m, 1H), 1.46 (s, 3H), 1.44 (s, 3H), 1.33-1.22 (m, 2H) | |
| 259 | ¹H NMR (400 MHz, DMSO-d₆) δ 11.07 (s, 1H), 10.00 (s, 1H), 8.78 (s, | B |
| | 1H), 8.63 (d, J = 8.4 Hz, 1H), 8.31 (d, J = 4.8 Hz, 1H), 7.73 (d, J = 8.4 | |
| | Hz, 1H), 7.58 (d, J = 3.6 Hz, 1H), 7.47 (d, J = 8.4 Hz, 1H), 7.26 (d, J = | |
| | 4.8 Hz, 1H), 6.96-6.89 (m, 2H), 6.86 (d, J = 8.4 Hz, 1H), 6.65 (d, J = | |
| | 8.4 Hz, 1H), 6.48 (d, J = 3.6 Hz, 1H), 5.27 (dd, J1 = 4.8, J2 = 12.4 Hz, | |
| | 1H), 4.65-4.55 (m, 1H), 4.45 (s, 2H), 3.87 (s, 3H), 3.63 (d, J = 10.4 | |
| | Hz, 2H), 2.85 (s, 5H), 2.74-2.57 (m, 8H), 2.48 (s, 3H), 2.42-2.35 (m, | |
| | 1H), 2.04-1.86 (m, 3H), 1.69-1.54 (m, 2H), 1.47 (s, 3H), 1.45 (s, 3H) | |
| 260 | ¹H NMR (400 MHz, DMSO-d₆) δ 11.06 (s, 1H), 9.85 (s, 1H), 8.70 ( d, | A |
| | J = 3.2 Hz, 1H), 8.46 (d, J = 8.4 Hz, 1H), 7.61 (s, 1H), 7.44 (dd, J = 8.4, | |
| | 14.2 Hz, 2H), 7.31-7.22 (m, 2H), 6.95-6.87 (m, 2H), 6.84 (d, J = 8.4 | |
| | Hz, 1H), 6.63 ( d, J = 8.8 Hz, 1H), 6.31 (d, J = 7.2 Hz, 1H), 5.26 ( dd, J = | |
| | 5.2, 12.7 Hz, 1H), 4.59 (td, J = 7.2, 13.6 Hz, 1H), 4.23 (s, 2H), 3.76 | |
| | (s, 3H), 3.56 ( d, J = 11.6 Hz, 2H), 2.92-2.80 (m, 5H), 2.74-2.52 (m, | |
| | 8H), 2.45 (s, 3H), 2.26 ( d, J = 6.8 Hz, 2H), 2.02-1.94 (m, 1H), 1.83 | |
| | (d, J = 11.6 Hz, 2H), 1.74-1.61 (m, 1H), 1.45 (d, J = 6.8 Hz, 6H), | |
| | 1.34-1.23 (m, 2H) | |
| 261 | ¹H NMR (400 MHz, DMSO-d₆) δ 11.05 (s, 1H), 9.94 (s, 1H), 8.79 (s, | A |
| | 1H), 8.65 (d, J = 8.6 Hz, 1H), 8.19-8.14 (m, 1H), 7.79 (s, 1H), 7.68 (d, | |
| | J = 8.4 Hz, 1H), 7.45 (d, J = 8.4 Hz, 1H), 6.95-6.87 (m, 2H), 6.86- | |
| | 6.79 (m, 2H), 6.63 (d, J = 8.4 Hz, 1H), 5.26 (dd, J = 5.2, 12.8 Hz, 1H), | |
| | 4.59 (td, J = 6.8, 13.8 Hz, 1H), 4.38 (s, 2H), 3.55 (d, J = 11.6 Hz, 4H), | |
| | 2.95-2.74 (m, 6H), 2.69-2.56 (m, 5H), 2.45 (s, 3H),2.32 (d, J = 1.6 | |
| | Hz, 3H), 2.25 (d, J = 6.8 Hz, 2H), 2.02-1.94 (m, 1H), 1.82 (d, J = 11.2 | |
| | Hz, 2H), 1.66 (s, 1H), 1.45 (d, J = 6.8 Hz, 6H), 1.34-1.22 (m, 2H) | |
| 262 | ¹H NMR (400 MHz, DMSO-d₆) δ 11.12 (s, 1H), 9.94 (s, 1H), 8.79 (s, | A |
| | 1H), 8.66 (d, J = 8.4 Hz, 1H), 8.44 (t, J = 6.4 Hz, 1H), 7.82 (s, 1H), 7.71 | |
| | (d, J = 8.4 Hz, 1H), 7.57-7.41 (m, 2H), 7.00-6.90 (m, 1H), 6.85 (d, | |
| | J = 8.4 Hz, 1H), 6.70-6.60 (m, 1H), 5.63-5.32 (m, 1H), 4.39 (s, 2H), | |
| | 3.51 (s, 5H), 3.09-2.92 (m, 2H), 2.85 (s, 4H), 2.70-2.60 (m, 3H), 2.59- | |
| | 2.53 (m, 3H), 2.47 (s, 3H), 2.33-2.17 (m, 3H), 2.16-2.04 (m, 1H), | |
| | 1.83 (d, J = 12.0 Hz, 2H), 1.70-1.60 (m, 1H), 1.40-1.20 (m, 2H) | |
| 263 | ¹H NMR (400 MHz, DMSO-d₆) δ 11.06 (s, 1H), 10.05 (s, 1H), 8.86 (s, | A |
| | 1H), 8.53 (d, J = 8.8 Hz, 1H), 8.49-8.40 (m, 1H), 8.05 (s, 1H), 7.83 (s, | |
| | 1H), 7.73 (d, J = 8.4 Hz, 1H), 7.53 (dd, J = 2.4, 10.0 Hz, 1H), 7.01- | |
| | 6.85 (m, 3H), 6.64 (d, J = 8.8 Hz, 1H), 5.30-5.24 (m, 1H), 4.62-4.56 | |
| | (m, 1H), 4.41 (s, 2H), 3.61-3.56 (m, 2H), 3.29-3.24 (m, 1H), 3.13 (s, | |
| | 4H), 2.96-2.81 (m, 2H), 2.71-2.53 (m, 6H), 2.52-2.51 (m, 3H), 2.49- | |
| | 2.44 (m, 2H), 2.03-1.95 (m, 1H), 1.84 (d, J = 12.0 Hz, 3H), 1.45 | |
| | (d, J = 6.8 Hz, 6H), 1.37-1.30 (m, 2H) | |
| 264 | ¹H NMR (400 MHz, DMSO-d₆) δ 11.07 (s, 1H), 9.96 (s, 1H), 8.83 (s, | A |
| | 1H), 8.76 (d, J = 8.4 Hz, 1H), 8.49-8.41 (m, 1H), 7.82 (s, 1H), 7.71 (d, | |
| | J = 8.4 Hz, 1H), 7.52 (dd, J = 2.4, 10.1 Hz, 1H), 6.99-6.87 (m, 3H), | |
| | 6.68-6.59 (m, 2H), 5.27 (dd, J = 5.2, 12.6 Hz, 1H), 4.59 (td, J = 6.8, | |
| | 13.8 Hz, 1H), 4.38 (s, 2H), 3.45-3.40 (m, 3H), 3.16 (t, J = 7.6 Hz, 2H), | |
| | 2.89-2.79 (m, 3H), 2.61 (t, J = 10.4 Hz, 6H), 2.48 (s, 3H), 2.33 (s, 2H), | |
| | 2.24 (d, J = 6.8 Hz, 2H), 2.01-1.94 (m, 1H), 1.83 (d, J = 11.2 Hz, 2H), | |
| | 1.66 (s, 1H), 1.45 (d, J = 6.8 Hz, 6H), 1.27 (d, J = 10.4 Hz, 2H), 1.19 (d, | |
| | J = 6.8 Hz, 6H) | |
| 265 | ¹H NMR (400 MHz, DMSO-d₆) δ 11.06 (s, 1H), 10.36 (s, 1H), 8.87 (s, | A |
| | 1H), 8.76 (d, J = 8.4 Hz, 1H), 8.50-8.42 (m, 1H), 8.32 (s, 1H), 8.14 (s, | |
| | 1H), 7.83 (s, 1H), 7.77 (d, J = 8.4 Hz, 1H), 7.53 (dd, J = 2.4, 10.0 Hz, | |
| | 1H), 7.01-6.84 (m, 3H), 6.67-6.61 (m, 1H), 5.26 (dd, J = 5.2, 12.6 | |
| | Hz, 1H), 4.59 (td, J = 7.0, 14.0 Hz, 1H), 4.40 (s, 2H), 3.56 (d, J = 11.6 | |
| | Hz, 3H), 3.02-2.84 (m, 6H), 2.73-2.60 (m, 5H), 2.45 (s, 3H), 2.35- | |
| | 2.27 (m, 2H), 1.98 (dd, J = 5.2, 10.8 Hz, 1H), 1.83 (d, J = 11.0 Hz, 2H), | |
| | 1.72-1.64 (m, 1H), 1.45 (d, J = 6.8 Hz, 6H), 1.34-1.23 (m, 2H) | |
| 266 | ¹H NMR (400 MHz, DMSO-d₆) δ 11.06 (s, 1H), 9.86 (s, 1H), 8.68 (s, | A |
| | 1H), 8.47 (d, J = 8.4 Hz, 1H), 7.67 (s, 1H), 7.45 (dd, J = 4.8, 8.4 Hz, | |
| | 2H), 7.20 (dd, J = 8.4, 10.8 Hz, 1H), 6.97-6.81 (m, 3H), 6.63 (dd, J = | |
| | 1.6, 8.8 Hz, 1H), 6.20 (dd, J = 3.2, 8.4 Hz, 1H), 5.26 (dd, J = 5.6, 12.8 | |
| | Hz, 1H), 4.59 (quin, J = 6.8 Hz, 1H), 4.24 (s, 2H), 3.74 (s, 3H), 3.56 (br | |
| | d, J = 11.6 Hz, 2H), 2.96-2.77 (m, 5H), 2.72-2.56 (m, 5H), 2.45 (s, | |
| | 3H), 2.26 (br d, J = 6.8 Hz, 2H), 2.05-1.93 (m, 1H), 1.83 (br d, J = | |
| | 11.6 Hz, 2H), 1.75-1.58 (m, 1H), 1.45 (d, J = 6.8 Hz, 6H), 1.36-1.18 | |
| | (m, 2H) | |
| 267 | ¹H NMR (400 MHz, DMSO-d₆) δ 10.59 (s, 1H), 9.94 (s, 1H), 8.79 (s, | A |
| | 1H), 8.65 (d, J = 8.8 Hz, 1H), 8.44 (t, J = 6.4 Hz, 1H), 8.16 (s, 1H), 8.03 | |
| | (d, J = 7.6 Hz, 1H), 7.81 (s, 1H), 7.70 (d, J = 8.8 Hz, 1H), 7.54-7.49 | |
| | (m, 1H), 7.47 (d, J = 8.4 Hz, 1H), 7.27 (s, 1H), 6.96 (dt, J = 2.8, 7.2 Hz, | |
| | 1H), 6.90-6.81 (m, 2H), 6.67 (s, 1H), 4.38 (s, 2H), 3.84-3.79 (m, 2H), | |
| | 3.75 (t, J = 6.8 Hz, 2H), 2.89-2.71 (m, 10H), 2.54 (d, J = 1.2 Hz, 2H), | |
| | 2.46 (s, 3H), 2.25 (d, J = 5.6 Hz, 2H), 1.87-1.74 (m, 3H), 1.30-1.18 | |
| | (m, 2H) | |
| 268 | ¹H NMR (400 MHz, DMSO-d₆) δ 10.62 (s, 1 H), 9.95 (s, 1 H), 8.81 (s, | A |
| | 1 H), 8.67 (d, J=8.4 Hz, 1 H), 8.45 (t, J-6.4 Hz, 1 H), 8.15 (s, 1 H), 8.08 | |
| | (s, 1 H), 7.83 (s, 1 H), 7.72 (d, J=8.4 Hz, 1 H), 7.45-7.55 (m, 2 H), | |
| | 7.35 (s, 1 H), 6.93-7.00 (m, 2 H), 6.85 (d, J=8.8 Hz, 1 H), 4.39 (s, 2 | |
| | H), 3.76 (t, J-6.4 Hz, 2 H), 3.23 (s, 2 H), 3.20 (d, J=2.0 Hz, 2 H), 2.87 | |
| | (s, 4 H), 2.83 (s, 2 H), 2.61-2.70 (m, 3 H), 2.47 (s, 3 H), 2.31 (d, J=7.2 | |
| | Hz, 3 H), 2.25 (s, 3 H), 1.88 (d, J=11.6 Hz, 2 H), 1.71-1.79 (m, 1 H), | |
| | 1.28-1.38 (m, 2 H) | |
| 269 | ¹H NMR (400 MHz, DMSO-d₆) δ 11.05 (s, 1H), 9.95 (s, 1H), 8.80 (s, | A |
| | 1H), 8.66 (d, J = 8.4 Hz, 1H), 8.28 (dd, J = 4.4, 7.2 Hz, 1H), 7.88 (s, | |
| | 1H), 7.70 (d, J = 8.4 Hz, 1H), 7.47 (d, J = 8.8 Hz, 1H), 7.14 (d, J = 8.8 | |
| | Hz, 1H), 6.92 (d, J = 8.4 Hz, 1H), 6.89 (d, J = 2.0 Hz, 1H), 6.85 (d, J = | |
| | 8.4 Hz, 1H), 6.63 (dd, J = 2.0, 8.8 Hz, 1H), 5.26 (dd, J = 5.2, 12.8 Hz, | |
| | 1H), 4.63-4.54 (m, 1H), 4.38 (s, 2H), 3.56 (d, J = 11.6 Hz, 2H), 2.92- | |
| | 2.81 (m, 5H), 2.66-2.51 (m, 8H), 2.46 (s, 3H), 2.26 (d, J = 6.8 Hz, 2H), | |
| | 2.02-1.94 (m, 1H), 1.83 (d, J = 10.8 Hz, 2H), 1.67 (s, 1H), 1.45 (d, J = | |
| | 6.8 Hz, 6H), 1.34-1.23 (m, 2H) | |
| 270 | ¹H NMR (400 MHz, DMSO-d₆) δ 11.05 (d, J = 5.6 Hz, 1H), 9.87 (d, J = | A |
| | 5.6 Hz, 1H), 8.79 (d, J = 5.2 Hz, 1H), 8.60-8.55 (m, 1H), 8.53 (d, J = | |
| | 6.4 Hz, 1H), 8.43 (d, J = 5.2 Hz, 1H), 8.18-8.10 (m, 2H), 8.04-7.97 | |
| | (m, 1H), 7.76-7.69 (m, 1H), 7.45 (dd, J = 3.2, 9.2 Hz, 1H), 7.25-7.16 | |
| | (m, 1H), 6.97 (dd, J = 6.0, 8.8 Hz, 1H), 6.94-6.86 (m, 2H), 6.63 (d, J = | |
| | 6.4 Hz, 1H), 5.30-5.22 (m, 1H), 4.62-4.55 (m, 1H), 4.43-4.37 (m, | |
| | 2H), 3.58-3.54 (m, 2H), 3.12 (s, 4H), 2.90-2.84 (m, 1H), 2.64 (d, J = | |
| | 12.0 Hz, 5H), 2.54-2.53 (m, 3H), 2.25 (d, J = 6.4 Hz, 2H), 2.01-1.95 | |
| | (m, 1H), 1.82 (td, J = 1.6, 9.2 Hz, 2H), 1.69 (dd, J = 3.2, 6.8 Hz, 1H), | |
| | 1.48-1.42 (m, 6H), 1.28 (dt, J = 1.2, 4.8 Hz, 2H) | |
| 271 | ¹H NMR (400 MHz, DMSO-d₆) δ 10.96 (s, 1H), 9.87 (s, 1H), 8.79 (s, | A |
| | 1H), 8.57 (d, J = 8.8 Hz, 1H), 8.52 (d, J = 7.2 Hz, 1H), 8.43 (s, 1H), | |
| | 8.13 (s, 1H), 7.99 (d, J = 2.8 Hz, 1H), 7.72 (d, J = 8.4 Hz, 1H), 7.44 (dd, | |
| | J = 2.8, 9.2 Hz, 1H), 7.37 (d, J = 8.0 Hz, 1H), 7.20 (dd, J = 1.6, 7.2 Hz, | |
| | 1H), 6.96 (d, J = 9.2 Hz, 1H), 6.58 (t, J = 8.0 Hz, 1H), 5.04 (dd, J = 5.2, | |
| | 13.3 Hz, 1H), 4.46-4.37 (m, 3H), 4.25 (d, J = 16.8 Hz, 1H), 4.08 (s, | |
| | 2H), 3.95 (s, 2H), 3.11 (s, 4H), 2.91-2.85 (m, 1H), 2.54 (s, 4H), 2.43 | |
| | (s, 4H), 2.35 (d, J = 12.4 Hz, 3H), 1.99-1.99 (m, 1H), 1.95-1.89 (m, | |
| | 2H) | |
| 272 | ¹H NMR (400 MHz, DMSO-d₆) δ 11.05 (s, 1H), 9.88 (s, 1H), 8.61 (s, | A |
| | 1H), 8.50 (d, J = 8.4 Hz, 1H), 7.95 (d, J = 2.0 Hz, 1H), 7.86 (d, J = 2.4 | |
| | Hz, 1H), 7.46 (d, J = 8.4 Hz, 2H), 7.06 (s, 1H), 6.97-6.79 (m, 3H), | |
| | 6.63 (dd, J = 1.6, 8.4 Hz, 1H), 5.26 (dd, J = 5.2, 12.4 Hz, 1H), 4.58 (td, | |
| | J = 6.8, 14.0 Hz, 1H), 4.41 (d, J = 18.8 Hz, 1H), 4.14-3.99 (m, 1H), | |
| | 3.83 (s, 3H), 3.63-3.45 (m, 2H), 2.92-2.76 (m, 5H), 2.73-2.53 (m, | |
| | 8H), 2.46 (s, 3H), 2.29-2.22 (m, 2H), 2.02-1.94 (m, 1H), 1.89-1.79 | |
| | (m, 2H), 1.72-1.62 (m, 1H), 1.45 (d, J = 6.8 Hz, 6H), 1.32-1.23 (m, | |
| | 2H) | |
| 273 | ¹H NMR (400 MHz, DMSO-d₆) δ 11.06 (s, 1H), 9.98 (s, 1H), 8.83 (s, | A |
| | 1H), 8.69 (d, J = 8.8 Hz, 1H), 8.40 (d, J = 7.2 Hz, 1H), 8.17 (s, 1H), | |
| | 7.73 (d, J = 8.4 Hz, 1H), 7.47 (d, J = 8.8 Hz, 1H), 7.22 (dd, J = 6.0, 7.2 | |
| | Hz, 1H), 6.92 (d, J = 8.8 Hz, 1H), 6.90-6.83 (m, 2H), 6.63 (dd, J = 1.6, | |
| | 8.4 Hz, 1H), 5.26 (dd, J = 5.2, 12.8 Hz, 1H), 4.63-4.54 (m, 1H), 4.40 | |
| | (s, 2H), 3.56 (d, J = 11.6 Hz, 2H), 2.85 (s, 5H), 2.62 (s, 4H), 2.59-2.52 | |
| | (m, 4H), 2.46 (s, 3H), 2.26 (d, J = 7.2 Hz, 2H), 2.01-1.93 (m, 1H), 1.83 | |
| | (d, J = 12.4 Hz, 2H), 1.71-1.62 (m, 1H), 1.45 (d, J = 6.9 Hz, 6H), 1.28 | |
| | (d, J = 10.4 Hz, 2H | |
| 274 | ¹H NMR (400 MHz, DMSO-d₆) δ 11.06 (s, 1H), 9.94 (s, 1H), 8.80 (s, | A |
| | 1H), 8.66 (d, J = 8.4 Hz, 1H), 8.44 (dd, J = 6.4, 7.2 Hz, 1H), 7.82 (s, | |
| | 1H), 7.71 (d, J = 8.4 Hz, 1H), 7.52 (dd, J = 2.4, 10.0 Hz, 1H), 7.47 (d, J = | |
| | 8.4 Hz, 1H), 7.11 (d, J = 12.0 Hz, 1H), 6.99-6.93 (m, 2H), 6.84 (d, J = | |
| | 8.8 Hz, 1H), 5.28 (dd, J = 5.2, 12.8 Hz, 1H), 4.63-4.54 (m, 1H), 4.38 | |
| | (s, 2H), 3.25 (d, J = 10.8 Hz, 4H), 2.92-2.80 (m, 5H), 2.69 (t, J = 10.4 | |
| | Hz, 3H), 2.63-2.56 (m, 3H), 2.46 (s, 3H), 2.36-2.26 (m, 2H), 2.02- | |
| | 1.93 (m, 1H), 1.84 (d, J = 11.2 Hz, 2H), 1.74-1.64 (m, 1H), 1.44 (d, J = | |
| | 6.8 Hz, 6H), 1.38-1.27 (m, 2H) | |
| 275 | ¹H NMR (400 MHz, DMSO-d₆) δ 11.05 (s, 1H), 9.88 (s, 1H), 8.73 (s, | A |
| | 1H), 8.53 (d, J = 8.4 Hz, 1H), 7.83 (s, 1H), 7.61 (dd, J = 4.4, 8.5 Hz, | |
| | 1H), 7.46 (d, J = 8.8 Hz, 1H), 7.27 (ddd, J = 4.4, 8.4, 10.4 Hz, 1H), 6.94- | |
| | 6.82 (m, 3H), 6.76-6.71 (m, 1H), 6.63 (d, J = 8.4 Hz, 1H), 5.26 (dd, J = | |
| | 5.2, 12.7 Hz, 1H), 4.58 (td, J = 7.2, 13.9 Hz, 1H), 4.35 (s, 2H), 3.56 | |
| | (d, J = 11.6 Hz, 3H), 2.93-2.78 (m, 6H), 2.69-2.55 (m, 6H), 2.45 (s, | |
| | 3H), 2.25 (d, J = 6.8 Hz, 2H), 2.01-1.95 (m, 1H), 1.82 (d, J = 11.6 Hz, | |
| | 2H), 1.69-1.62 (m, 1H), 1.45 (d, J = 6.8 Hz, 6H), 1.32-1.23 (m, 2H) | |
| 276 | ¹H NMR (400 MHz, DMSO-d₆) δ 11.06 (s, 1H), 9.97 (s, 1H), 8.82 (s, | A |
| | 1H), 8.68 (d, J = 8.4 Hz, 1H), 8.55 (d, J = 7.2 Hz, 1H), 8.43 (d, J = 0.8 | |
| | Hz, 1H), 8.14 (s, 1H), 7.75 (d, J = 8.4 Hz, 1H), 7.46 (d, J = 8.4 Hz, 1H), | |
| | 7.20 (dd, J = 1.6, 7.2 Hz, 1H), 6.92 (d, J = 8.4 Hz, 1H), 6.87-6.80 (m, | |
| | 2H), 6.63 (dd, J = 2.0, 8.4 Hz, 1H), 5.28 (dd, J = 5.2, 12.8 Hz, 1H), 4.40 | |
| | (s, 2H), 3.57 (d, J = 12.0 Hz, 2H), 3.30 (s, 3H), 2.95-2.88 (m, 1H), | |
| | 2.84 (s, 4H), 2.73-2.65 (m, 2H), 2.63-2.57 (m, 4H), 2.54 (s, 1H), 2.46 | |
| | (s, 3H), 2.44-2.38 (m, 3H), 2.03-1.94 (m, 1H), 1.79 (d, J = 11.6 Hz, | |
| | 2H), 1.50-1.40 (m, 3H), 1.37-1.26 (m, 2H) | |
| 277 | ¹H NMR (400 MHz, DMSO-d₆) δ 11.09 (s, 1H), 9.97 (s, 1H), 8.82 (s, | A |
| | 1H), 8.68 (d, J = 8.4 Hz, 1H), 8.55 (d, J = 7.2 Hz, 1H), 8.44 (s, 1H), | |
| | 8.15 (s, 1H), 7.75 (d, J = 8.4 Hz, 1H), 7.47 (d, J = 8.8 Hz, 1H), 7.20 (dd, | |
| | J = 1.6, 7.2 Hz, 1H), 7.00-6.94 (m, 1H), 6.92 (s, 1H), 6.86 (dd, J = 4.4, | |
| | 8.0 Hz, 2H), 5.35 (dd, J = 5.2, 12.4 Hz, 1H), 4.41 (s, 2H), 3.63 (s, 3H), | |
| | 3.10 (d, J = 9.6 Hz, 2H), 2.85 (s, 5H), 2.72-2.62 (m, 4H), 2.61-2.53 | |
| | (m, 4H), 2.46 (s, 3H), 2.42 (s, 2H), 2.04-1.95 (m, 1H), 1.80 (d, J = | |
| | 10.4 Hz, 2H), 1.53-1.35 (m, 5H) | |
| 278 | ¹H NMR (400 MHz, DMSO-d₆) δ 10.99 (s, 1H), 9.93 (s, 1H), 8.79 (s, | A |
| | 1H), 8.65 (d, J = 8.4 Hz, 1H), 8.44 (dd, J = 6.0, 7.2 Hz, 1H), 8.15 (s, | |
| | 1H), 7.81 (s, 1H), 7.70 (d, J = 8.6 Hz, 1H), 7.58-7.39 (m, 3H), 7.15 (t, | |
| | J = 7.9Hz, 1H), 6.96 (dt, J = 2.8, 7.6 Hz, 1H), 6.83 (d, J = 8.6 Hz, 1H), | |
| | 5.07 (dd, J = 5.2, 13.2 Hz, 1H), 4.54-4.23 (m, 4H), 3.60 (d, J = 7.6 Hz, | |
| | 1H), 3.42-3.29 (m, 4H), 2.98-2.87 (m, 3H), 2.82 (s, 4H), 2.64-2.52 | |
| | (m, 2H), 2.44 (s, 3H), 2.43-2.31 (m, 2H), 2.14 (d,J = 6.8 Hz, 2H), 2.04- | |
| | 1.86 (m, 5H), 1.79 (d, J = 11.2 Hz, 2H), 1.64-1.41 (m, 3H), 1.28- | |
| | 1.06 (m, 2H), 1.02-0.77 (m, 2H) | |
| 279 | ¹H NMR (400 MHz, DMSO-d₆) δ 11.09 (s, 1H), 9.93 (s, 1H), 8.79 (s, | A |
| | 1H), 8.65 (d, J = 8.6 Hz, 1H), 8.49-8.39 (m, 1H), 8.16 (s, 1H), 7.81 (s, | |
| | 1H), 7.70 (d, J = 8.6 Hz, 1H), 7.51 (dd, J = 2.4, 10.0 Hz, 1H), 7.45 (d, | |
| | J = 8.6Hz, 1H), 7.00-6.92 (m, 2H), 6.91-6.78 (m, 3H), 5.35 (dd, J = | |
| | 5.2, 12.6 Hz, 1H), 4.38 (s, 2H), 3.91-3.70 (m, 1H), 3.62 (s, 3H), 3.59- | |
| | 3.19 (m, 5H), 3.17-2.56 (m, 12H), 2.44 (s, 3H), 2.14 (d, J = 6.8 Hz, | |
| | 2H), 2.01-1.90 (m, 4H), 1.80 (d, J = 11.2 Hz, 2H), 1.59-1.41 (m, 2H), | |
| | 1.16 (d, J = 10.8 Hz, 2H), 0.91-0.88 (m, 2H) | |
| 280 | ¹H NMR (400 MHz, DMSO-d₆) δ 11.06 (s, 1H), 9.85 (s, 1H), 8.76 (s, | A |
| | 1H), 8.56 (d, J = 8.4 Hz, 1H), 8.41 (dd, J = 6.0, 7.2 Hz, 1H), 8.13 (s, | |
| | 1H), 8.01 (d, J = 2.8 Hz, 1H), 7.80 (s, 1H), 7.68 (d, J = 8.8 Hz, 1H), | |
| | 7.52 (dd, J = 2.4, 10.0 Hz, 1H), 7.45 (dd, J = 2.8, 9.2 Hz, 1H), 7.00- | |
| | 6.90 (m, 3H), 6.83 (d, J = 2.0 Hz, 1H), 6.64 (dd, J = 2.0, 8.8 Hz, 1H), | |
| | 5.29 (dd, J = 5.2, 12.8 Hz, 1H), 4.37 (s, 2H), 3.60 (d, J = 12.0 Hz, 2H), | |
| | 3.31 (s, 3H), 3.16 (s, 4H), 2.94-2.85 (m, 1H), 2.72-2.58 (m, 8H), 2.42- | |
| | 2.29 (m,2H), 2.02-1.94 (m, 1H), 1.83 (d, J = 12.4 Hz, 2H), 1.73 (d, | |
| | J = 1.6 Hz, 1H), 1.36-1.22 (m, 2H) | |
| 281 | ¹H NMR (400 MHz, DMSO-d₆) δ 11.06 (s, 1H), 9.88 (s, 1H), 8.79 (s, | A |
| | 1H), 8.61-8.49 (m, 2H), 8.43 (s, 1H), 8.13 (d, J = 1.6 Hz, 1H), 8.01 (s, | |
| | 1H), 7.73 (d, J = 8.4 Hz, 1H), 7.51-7.41 (m, 1H), 7.20 (d, J = 7.2 Hz, | |
| | 1H), 6.95 (dd, J = 8.0, 19.6 Hz, 2H), 6.82 (s, 1H), 6.71-6.58 (m, 1H), | |
| | 5.33-5.24 (m, 1H), 4.39 (s, 2H), 3.59 (d, J = 9.6 Hz, 2H), 3.31 (s, 3H), | |
| | 3.15 (s, 4H), 2.92-2.84 (m, 2H), 2.66-2.60 (m, 4H), 2.56-2.53 (m, | |
| | 3H), 2.32 (d, J = 4.0 Hz, 2H), 2.02-1.95 (m, 1H), 1.83 (d, J = 11.2 Hz, | |
| | 2H), 1.72 (s, 1H), 1.34-1.22 (m, 2H) | |
| 282 | ¹H NMR (400 MHz, DMSO-d₆) δ 11.09 (s, 1H), 9.88 (s, 1H), 8.79 (s, | A |
| | 1H), 8.58 (d, J = 8.4 Hz, 1H), 8.55-8.50 (m, 1H), 8.45-8.42 (m, 1H), | |
| | 8.14 (s, 1H), 8.01 (d, J = 2.8 Hz, 1H), 7.73 (d, J = 8.8 Hz, 1H), 7.45 (dd, | |
| | J = 2.8, 9.2 Hz, 1H), 7.20 (dd, J = 1.6, 7.2 Hz, 1H), 6.97 (dd, J = 2.0, 8.4 | |
| | Hz, 2H), 6.90 (d, J = 7.6 Hz, 1H), 6.87 (d, J = 8.0 Hz, 1H), 5.35 (dd, J = | |
| | 5.2, 12.4 Hz, 1H), 4.40 (s, 2H), 3.64 (s, 3H), 3.13 (s, 4H), 2.93-2.83 | |
| | (m, 2H), 2.72-2.64 (m, 4H), 2.53 (dd, J = 2.0, 3.6 Hz, 3H), 2.46 (d, J = | |
| | 2.0 Hz, 2H), 2.28 (d, J = 6.0 Hz, 2H), 2.03-1.96 (m, 1H), 1.85 (d, J = | |
| | 11.6 Hz, 2H), 1.76-1.62 (m, 1H), 1.42-1.28 (m, 2H) | |
| 283 | ¹H NMR (400 MHz, DMSO-d₆) δ 11.07 (s, 1H), 9.97 (s, 1H), 8.82 (s, | A |
| | 1H), 8.68 (d, J = 8.4 Hz, 1H), 8.55 (d, J = 7.2 Hz, 1H), 8.43 (s, 1H), | |
| | 8.14 (s, 1H), 7.75 (d, J = 8.8 Hz, 1H), 7.47 (d, J = 8.4 Hz, 1H), 7.20 (dd, | |
| | J = 1.6, 7.2 Hz, 1H), 6.93 (d, J = 8.8 Hz, 1H), 6.87-6.80 (m, 2H), 6.63 | |
| | (dd, J = 2.0, 8.8 Hz, 1H), 5.28 (dd, J = 5.2, 12.8 Hz, 1H), 4.40 (s, 2H), | |
| | 3.59 (d, J = 12.0 Hz, 2H), 3.30 (s, 3H), 2.90-2.81 (m, 5H), 2.69-2.58 | |
| | (m, 5H), 2.52 (s, 3H), 2.46 (s, 3H), 2.25 (d, J = 7.2 Hz, 2H), 2.02-1.95 | |
| | (m, 1H), 1.82 (d, J = 12.0 Hz, 2H), 1.68 (d, J = 2.0 Hz, 1H), 1.34-1.20 | |
| | (m, 2H) | |
| 284 | ¹H NMR (400 MHz, DMSO-d₆) δ 10.59 (s, 1H), 9.84 (s, 1H), 8.76 (s, | A |
| | 1H), 8.56 (d, J = 8.8 Hz, 1H), 8.41 (dd, J = 6.0, 7.6 Hz, 1H), 8.19 (s, | |
| | 1H), 8.07-7.97 (m, 2H), 7.80 (s, 1H), 7.68 (d, J = 8.8 Hz, 1H), 7.55- | |
| | 7.39 (m, 2H), 7.27 (s, 1H), 7.00-6.93 (m, 2H), 6.87 (dd, J = 2.0, 7.6 | |
| | Hz, 1H), 6.69-6.62 (m, 1H), 4.37 (s, 2H), 3.86-3.72 (m, 5H), 3.12 (s, | |
| | 6H), 2.87-2.69 (m, 5H), 2.23 (d, J = 6.0 Hz, 2H), 1.86-1.76 (m, 3H), | |
| | 1.29-1.17 (m, 2H) | |
| 285 | ¹H NMR (400 MHz, DMSO-d₆) δ 11.05 (s, 1H), 10.77 (s, 1H), 9.95 (s, | A |
| | 1H), 8.80 (s, 1H), 8.66 (d, J = 8.4 Hz, 1H), 8.49-8.35 (m, 1H), 8.17- | |
| | 8.09 (m, 1H), 7.81 (s, 1H), 7.70 (d, J = 8.4 Hz, 1H), 7.56-7.41 (m, 2H), | |
| | 7.02-6.92 (m, 1H), 6.92-6.78 (m, 2H), 6.65-6.54 (m, 2H), 5.22 (dd, J = | |
| | 5.2, 12.8 Hz, 1H), 4.38 (s, 2H), 3.53-3.48 (m, 4H), 2.89 (s, 5H), 2.80- | |
| | 2.66 (m, 4H), 2.65-2.56 (m, 4H), 2.46 (s, 3H), 2.02-1.93 (m, 1H), | |
| | 1.82 (d, J = 12.0 Hz, 2H), 1.76-1.65 (m, 1H), 1.35-1.21 (m, 2H) | |
| 286 | ¹H NMR (400 MHz, DMSO-d₆) δ 10.56 (s, 1H), 9.94 (s, 1H), 9.32 (s, | A |
| | 1H), 8.79 (s, 1H), 8.69-8.61 (m, 1H), 8.50-8.39 (m, 2H), 7.81 (s, 1H), | |
| | 7.78-7.64 (m, 3H), 7.58-7.40 (m, 2H), 6.96 (dt, J = 2.4, 7.6 Hz, 1H), | |
| | 6.84 (d, J = 8.8 Hz, 1H), 4.38 (s, 2H), 3.98-3.86 (m, 1H), 3.70 (td, J = | |
| | 6.0, 12.0 Hz, 1H), 3.61-3.55 (m, 2H), 2.93 (dd, J = 6.0, 10.0 Hz, 3H), | |
| | 2.86 (s, 4H), 2.80-2.66 (m, 2H), 2.60-2.54 (m, 3H), 2.46 (s, 3H), 2.29 | |
| | (d, J = 6.8 Hz, 2H), 1.88 (d, J = 12.4 Hz, 2H), 1.82-1.70 (m, 1H), | |
| | 1.44-1.28 (m, 2H) | |
| 287 | ¹H NMR (400 MHz, DMSO-d₆) δ 10.53 (s, 1H), 9.94 (s, 1H), 9.40 (s, | A |
| | 1H), 8.79 (s, 1H), 8.66 (d, J = 8.4 Hz, 1H), 8.52 (s, 1H), 8.44 (t, J = 6.4 | |
| | Hz, 1H), 7.82 (s, 1H), 7.71 (d, J = 8.0 Hz, 2H), 7.59 (d, J = 8.4 Hz, 1H), | |
| | 7.54-7.46 (m, 2H), 7.26 (d, J = 7.2 Hz, 1H), 6.99-6.93 (m, 1H), 6.85 | |
| | (d, J = 8.4 Hz, 1H), 4.38 (s, 2H), 3.93-3.87 (m, 1H), 3.70 (td, J = 5.6, | |
| | 11.9 Hz, 2H), 2.87 (s, 5H), 2.84-2.73 (m, 3H), 2.58 (s, 5H), 2.47 (s, | |
| | 3H), 2.36 (d, J = 6.4 Hz, 2H), 1.94 (d, J = 11.6 Hz, 2H), 1.84-1.74 (m, | |
| | 1H), 1.61-1.48 (m, 2H) | |
| 288 | ¹H NMR (400 MHz, DMSO-d₆) δ 10.63 (s, 1H), 9.93 (s, 1H), 8.79 (s, | A |
| | 1H), 8.66 (d, J = 8.4 Hz, 1H), 8.44 (t, J = 6.8 Hz, 1H), 8.16 (s, 1H), 8.05 | |
| | (dd, J = 2.0, 3.6 Hz, 1H), 7.81 (s, 1H), 7.70 (d, J = 8.8 Hz, 1H), 7.54- | |
| | 7.45 (m, 3H), 6.96 (dt, J = 2.4, 7.6 Hz, 1H), 6.84 (d, J = 8.8 Hz, 1H), | |
| | 6.53 (dd, J = 2.0, 11.6 Hz, 1H), 4.50-4.41 (m, 2H), 4.38 (s, 2H), 3.81- | |
| | 3.66 (m, 3H), 2.93-2.74 (m, 9H), 2.56-2.53 (m, 2H), 2.46 (s, 3H), | |
| | 2.27 (d, J = 6.0 Hz, 2H), 1.89-1.78 (m, 3H), 1.36-1.22 (m, 2H) | |
| 289 | ¹H NMR (400 MHz, DMSO-d₆) δ 11.05 (s, 1H), 9.98 (s, 1H), 8.82 (s, | A |
| | 1H), 8.68 (d, J = 8.4 Hz, 1H), 8.52 (d, J = 7.2 Hz, 1H), 8.22-8.16 (m, | |
| | 1H), 7.74 (d, J = 8.4 Hz, 1H), 7.48 (d, J = 8.8 Hz, 1H), 7.29 (d, J = 7.2 | |
| | Hz, 1H), 6.98-6.82 (m, 3H), 6.69-6.58 (m, 1H), 5.26 (dd, J = 5.2, | |
| | 12.8 Hz, 1H), 4.59 (quin, J = 6.8 Hz, 1H), 4.40 (s, 2H), 3.56 (d, J = 11.2 | |
| | Hz, 2H), 2.91-2.79 (m, 5H), 2.71-2.52 (m, 8H), 2.46 (s, 3H), 2.26 (d, | |
| | J = 6.4 Hz, 2H), 2.03-1.93 (m, 1H), 1.83 (d, J = 11.6 Hz, 2H), 1.75- | |
| | 1.59 (m, 1H), 1.45 (d, J = 6.8 Hz, 6H), 1.35-1.20 (m, 2H) | |
| 290 | ¹H NMR (400 MHz, DMSO-d₆) δ 10.60 (s, 1H), 9.96 (s, 1H), 8.80 (s, | A |
| | 1H), 8.64 (d, J = 8.4 Hz, 1H), 8.48-8.40 (m, 1H), 8.16 (s, 1H), 8.08 (d, | |
| | J = 7.6 Hz, 1H), 7.80 (s, 1H), 7.72 (d, J = 8.8 Hz, 1H), 7.56-7.44 (m, | |
| | 2H), 7.40 (s, 1H), 6.96 (m, J = 2.8, 7.6 Hz, 1H), 6.84 (t, J = 8.8 Hz, 2H), | |
| | 4.40 (s, 2H), 3.76 (t, J = 6.8 Hz, 2H), 3.12 (d, J = 11.2 Hz, 2H), 2.88- | |
| | 2.72 (m, 8H), 2.56 (d, J = 7.2 Hz, 3H), 2.48 (s, 3H), 2.40 (s, 3H), 2.32 | |
| | (d, J = 6.8 Hz, 2H), 1.88 (d, J = 12.0 Hz, 2H), 1.72 (s, 1H), 1.40-1.28 | |
| | (m, 2H) | |
| 291 | ¹H NMR (400 MHz, DMSO-d₆) δ 10.45 (s, 1H), 9.94 (s, 1H), 9.00 (s, | A |
| | 1H), 8.79 (s, 1H), 8.66 (d, J = 8.4 Hz, 1H), 8.50-8.37 (m, 1H), 7.87 (s, | |
| | 1H), 7.83-7.76 (m, 2H), 7.71 (d, J = 8.8 Hz, 1H), 7.67-7.62 (m, 1H), | |
| | 7.54-7.45 (m, 2H), 7.32 (s, 1H), 6.96 (dt, J = 2.8, 7.2 Hz, 1H), 6.85 (d, | |
| | J = 8.8 Hz, 1H), 4.38 (s, 2H), 4.05 (t, J = 6.4 Hz, 2H), 3.88 (d, J = 12.8 | |
| | Hz, 2H), 2.87-2.76 (m, 6H), 2.75-2.65 (m, 3H), 2.54 (d, J = 1.6 Hz, | |
| | 2H), 2.46 (s, 3H), 2.35-2.19 (m, 3H), 1.87 (d, J = 12.4 Hz, 2H), 1.83- | |
| | 1.72 (m, 1H), 1.34-1.22 (m, 2H) | |
| 292 | ¹H NMR (400 MHz, DMSO-d₆) δ 10.53 (s, 1H), 9.93 (s, 1H), 9.27 (s, | A |
| | 1H), 8.79 (s, 1H), 8.66 (d, J = 8.4 Hz, 1H), 8.47-8.39 (m, 1H), 8.04 (s, | |
| | 1H), 7.81 (s, 1H), 7.70 (d, J = 8.4 Hz, 1H), 7.66-7.60 (m, 1H), 7.56- | |
| | 7.45 (m, 3H), 7.11 (d, J = 7.6 Hz, 1H), 6.96 (dt, J = 2.4, 7.2 Hz, 1H), | |
| | 6.84 (d, J = 8.8 Hz, 1H), 4.38(s, 2H), 4.14 (t, J = 6.4 Hz, 2H), 2.92- | |
| | 2.70 (m, 9H), 2.58 ( s, 5H), 2.47 (s, 3H), 2.37-2.32 (m, 2H), 1.93 ( d, | |
| | J = 10.4 Hz, 2H), 1.85-1.73 (m, 1H), 1.59-1.45 (m, 2H) | |
| 293 | ¹H NMR (400 MHz, DMSO-d₆) δ 11.06 (s, 1H), 9.97 (s, 1H), 8.82 (s, | A |
| | 1H), 8.68 (d, J = 8.4 Hz, 1H), 8.55 (d, J = 7.2 Hz, 1H), 8.44 (d, J = 0.8 | |
| | Hz, 1H), 8.15 (s, 1H), 7.75 (d, J = 8.8 Hz, 1H), 7.47 (d, J = 8.8 Hz, 1H), | |
| | 7.20 (dd, J = 1.6, 7.2 Hz, 1H), 6.97-6.80 (m, 3H), 6.63 (dd, J = 2.0, 8.4 | |
| | Hz, 1H), 5.26 (dd, J = 5.2, 12.8 Hz, 1H), 4.58 (td, J = 6.8, 14.0 Hz, 1H), | |
| | 4.41 (s, 2H), 3.54 (d, J = 12.0 Hz, 3H), 2.96-2.79 (m, 6H), 2.67 (d, J = | |
| | 4.0 Hz, 1H), 2.59 (d, J = 11.2 Hz, 5H), 2.46 (s, 3H), 2.41 (d, J = 6.4 Hz, | |
| | 2H), 2.02-1.93 (m, 1H), 1.79 (d, J = 12.0 Hz, 2H), 1.45 (d, J = 6.8 Hz, | |
| | 9H), 1.37-1.25 (m, 2H) | |
| 294 | ¹H NMR (400 MHz, DMSO-d₆) δ 11.07 (s, 1H), 9.92 (s, 1H), 8.78 (s, | A |
| | 1H), 8.65 (d, J = 8.4 Hz, 1H), 8.43 (dd, J = 6.0, 7.2 Hz, 1H), 7.81 (s, | |
| | 1H), 7.70 (d, J = 8.4 Hz, 1H), 7.57-7.40 (m, 2H), 7.05-6.91 (m, 2H), | |
| | 6.84 (d, J = 8.4 Hz, 1H), 6.79 (br s, 1H), 6.69 (dd, J = 1.6, 8.4 Hz, 1H), | |
| | 5.33 (dd, J = 5.2, 12.4 Hz, 1H), 4.37 (s, 2H), 3.66-3.48 (m, 5H), 3.27 | |
| | (s, 3H), 2.90-2.57 (m, 10H), 2.45 (s, 3H), 2.25 (br d, J = 7.2 Hz, 2H), | |
| | 1.99 (br d, J = 5.2 Hz, 1H), 1.82 (br d, J = 12.4 Hz, 2H), 1.73-1.54 (m, | |
| | 1H), 1.26 (br d, J = 12.4 Hz, 2H) | |
| 304 | ¹H NMR (400 MHz, DMSO-d6) δ 11.06 (s, 1H), 9.89 (s, 1H), 8.65 (s, | A |
| | 1H), 8.59 (d, J = 8.4 Hz, 1H), 7.96 (d, J = 8.4 Hz, 1H), 7.81 (dd, J = 2.4, | |
| | 10.0 Hz, 1H), 7.70 (dd, J = 6.0, 9.2 Hz, 1H), 7.62 (t, J = 7.6 Hz, 1H), | |
| | 7.52 (d, J = 6.8 Hz, 1H), 7.46 (dd, J = 2.4, 8.4 Hz, 2H), 7.38 (dt, J = 2.4, | |
| | 8.8 Hz, 1H), 6.93 (d, J = 8.4 Hz, 1H), 6.86 (d, J = 8.4 Hz, 1H), 6.82 (d, J = | |
| | 1.6 Hz, 1H), 6.63 (dd, J = 1.6, 8.4 Hz, 1H), 5.29 (dd, J = 5.2, 12.8 Hz, | |
| | 1H), 4.23 (s, 1H), 3.94 (s, 1H), 3.59 (d, J = 11.8 Hz, 2H), 3.36 (s, 4H), | |
| | 2.96-2.81 (m, 5H), 2.70-2.54 (m, 7H), 2.45 (s, 3H), 2.28 (d, J = 6.8 | |
| | Hz, 2H), 2.03-1.93 (m, 1H), 1.83 (d, J = 12.0 Hz, 2H), 1.69 (d, J = 10.8 | |
| | Hz, 1H), 1.35-1.21 (m, 2H) | |
| 305 | ¹H NMR (400 MHz, DMSO-d6) δ11.05 (s, 1H), 9.97 (s, 1H), 9.09 (d, J | A |
| | = 4.4 Hz, 1H), 8.74 (s, 1H), 8.66 (d, J = 8.4 Hz, 1H), 8.23 (d, J = 7.2 Hz, | |
| | 1H), 8.07 (d, J = 8.4 Hz, 1H), 7.77 (d, J = 4.4 Hz, 1H), 7.71 (t, J = 8.0 | |
| | Hz, 1H), 7.55 (d, J = 8.4 Hz, 1H), 7.47 (d, J = 8.4 Hz, 1H), 6.94-6.85 | |
| | (m, 3H), 6.66-6.60 (m, 1H), 5.30-5.23 (m, 1H), 4.65-4.52 (m, 1H), | |
| | 4.23 (s, 2H), 3.55 (d, J = 11.6 Hz, 3H), 2.94-2.81 (m, 5H), 2.73-2.52 | |
| | (m, 7H), 2.46 (s, 3H), 2.25 (d, J = 7.2 Hz, 2H), 2.03-1.93 (m, 1H), 1.82 | |
| | (d, J = 11.2 Hz, 2H), 1.73-1.59 (m, 1H), 1.45 (d, J = 6.8 Hz, 6H), 1.28 | |
| | (d, J = 10.0 Hz, 2H) | |
| 306 | ¹H NMR (400 MHz, DMSO-d6) δ11.07 (s, 1H), 9.88 (s, 1H), 8.63 (s, | A |
| | 1H), 8.58 (d, J = 8.4 Hz, 1H), 7.99 (dd, J = 8.0, 16.4 Hz, 2H), 7.63 (s, | |
| | 1H), 7.61-7.52 (m, 3H), 7.51-7.44 (m, 3H), 6.93 (d, J = 8.4 Hz, 1H), | |
| | 6.89-6.80 (m, 2H), 6.64 (dd, J = 1.6, 8.4 Hz, 1H), 5.29 (dd, J = 5.6, | |
| | 12.8 Hz, 1H), 4.33-4.17 (m, 1H), 4.00-3.86 (m, 1H), 3.59 (d, J = 11.6 | |
| | Hz, 3H), 3.30 (s, 3H), 2.85 (s, 4H), 2.73-2.61 (m, 4H), 2.61-2.52 (m, | |
| | 4H), 2.46 (s, 3H), 2.26 (d, J = 6.8 Hz, 2H), 2.02-1.94 (m, 1H), 1.83 (d, | |
| | J = 12.8 Hz, 2H), 1.67 (dd, J = 2.4, 4.4 Hz, 1H), 1.27 (q, J = 11.6 Hz, | |
| | 2H) | |
| 307 | ¹H NMR (400 MHz, DMSO-d6) δ11.05 (s, 1H), 9.96 (s, 1H), 8.99 (d, J | A |
| | = 4.4 Hz, 1H), 8.73 (s, 1H), 8.64 (d, J = 8.4 Hz, 1H), 7.70 (d, J = 4.4 Hz, | |
| | 1H), 7.66-7.53 (m, 4H), 7.47 (d, J = 8.4 Hz, 1H), 6.94-6.84 (m, 3H), | |
| | 6.63 (dd, J = 1.6, 8.4 Hz, 1H), 5.26 (dd, J = 5.6, 12.8 Hz, 1H), 4.64- | |
| | 4.53 (m, 1H), 4.36-4.05 (m, 2H), 3.56 (d, J = 11.6 Hz, 2H), 2.92-2.82 | |
| | (m, 5H), 2.73-2.52 (m, 8H), 2.46 (s, 3H), 2.26 (d, J = 7.2 Hz, 2H), 2.02- | |
| | 1.93 (m, 1H), 1.83 (d, J = 11.8 Hz, 2H), 1.67 (d, J = 2.4 Hz, 1H), 1.45 | |
| | (d, J = 6.8 Hz, 6H), 1.35-1.22 (m, 2H) | |
| 308 | ¹H NMR (400 MHz, DMSO-d6) δ11.06 (s, 1H), 9.95 (s, 1H), 8.96 (d, J = | A |
| | 4.4 Hz, 1H), 8.74 (s, 1H), 8.64 (d, J = 8.4 Hz, 1H), 7.88-7.82 (m, | |
| | 2H), 7.60 (d, J = 4.4 Hz, 1H), 7.56-7.45 (m, 3H), 6.95-6.84 (m, 3H), | |
| | 6.63 (d, J = 7.6 Hz, 1H), 5.26 (dd, J = 5.2, 12.4 Hz, 1H), 4.65-4.53 (m, | |
| | 1H), 4.37-4.09 (m, 2H), 3.56 (d, J = 11.2 Hz, 3H), 2.85 (s, 5H), 2.69- | |
| | 2.61 (m, 4H), 2.56-2.53 (m, 3H), 2.46 (s, 3H), 2.26 (d, J = 7.2 Hz, 2H), | |
| | 1.98 (dd, J = 4.4, 10.4 Hz, 1H), 1.83 (d, J = 12.4 Hz, 2H), 1.71-1.62 | |
| | (m, 1H), 1.45 (d, J = 6.8 Hz, 6H), 1.28 (d, J = 9.6 Hz, 2H) | |
| 309 | ¹H NMR (400 MHz, DMSO-d6) δ11.05 (s, 1H), 9.99 (s, 1H), 9.47 (s, | A |
| | 1H), 9.10 (d, J = 4.4 Hz, 1H), 8.76 (s, 1H), 8.66 (d, J = 8.4 Hz, 1H), 8.60 | |
| | (d, J = 6.0 Hz, 1H), 7.89 (d, J = 4.4 Hz, 1H), 7.69 (d, J = 6.0 Hz, 1H), | |
| | 7.58 (d, J = 8.4 Hz, 1H), 7.48 (d, J = 8.4 Hz, 1H), 6.95-6.84 (m, 3H), | |
| | 6.63 (d, J = 8.8 Hz, 1H), 5.30-5.22 (m, 1H), 4.64-4.54 (m, 1H), 4.28 | |
| | (s, 2H), 3.56 (d, J = 11.6 Hz, 2H), 2.95-2.80 (m, 5H), 2.73-2.51 (m, | |
| | 8H), 2.46 (s, 3H), 2.26 (d, J = 6.8 Hz, 2H), 1.98 (d, J = 11.2 Hz, 1H), | |
| | 1.83 (d, J = 12.0 Hz, 2H), 1.73-1.59 (m, 1H), 1.45 (d, J = 6.8 Hz, 6H), | |
| | 1.28 (d, J = 10.0 Hz, 2H) | |
| 310 | ¹H NMR (400 MHz, DMSO-d6) δ 10.65 (s, 1H), 9.92 (s, 1H), 8.77 (s, | A |
| | 1H), 8.64 (d, J = 8.4 Hz, 1H), 8.43 (t, J = 6.4 Hz, 1H), 8.14 (s, 1H), 8.02 | |
| | (d, J = 7.2 Hz, 1H), 7.80 (s, 1H), 7.70 (d, J = 8.8 Hz, 1H), 7.52-7.44 | |
| | (m, 2H), 7.43 (s, 1H), 6.99-6.93 (m, 1H), 6.89-6.81 (m, 2H), 4.37 (s, | |
| | 2H), 3.76 (d, J = 6.4 Hz, 2H), 2.93-2.79 (m, 10H), 2.54 (d, J = 3.6 Hz, | |
| | 4H), 2.46 (s, 3H), 2.27 (d, J = 6.8 Hz, 2H), 1.83 (d, J = 12.4 Hz, 2H), | |
| | 1.75 (dd, J = 4.4, 5.6 Hz, 1H), 1.35-1.24 (m, 2H) | |
| 311 | ¹H NMR (400 MHz, DMSO-d6) δ 13.53 (s, 1H), 11.05 (s, 1H), 9.93 (s, | A |
| | 1H), 8.95 (d, J = 8.8 Hz, 1H), 8.80 (s, 1H), 8.64 (d, J = 1.2 Hz, 1H), 8.49 | |
| | (d, J = 8.4 Hz, 1H), 8.11-7.89 (m, 2H), 7.45 (d, J = 7.6 Hz, 1H), 7.02- | |
| | 6.95 (m, 1H), 6.95-6.86 (m, 2H), 6.64 (d, J = 8.0 Hz, 1H), 5.26 (dd, J = | |
| | 5.2, 12.8 Hz, 1H), 4.71 (s, 2H), 4.63-4.55 (m, 1H), 3.57 (d, J = 9.6 Hz, | |
| | 3H), 3.13 (s, 4H), 2.95-2.83 (m, 2H), 2.71-2.56 (m, 7H), 2.25 (s, 1H), | |
| | 2.02-1.95 (m, 1H), 1.84 (d, J = 11.6 Hz, 2H), 1.74-1.60 (m, 1H), 1.45 | |
| | (d, J = 6.8 Hz, 6H), 1.29 (dd, J = 2.4, 6.4 Hz, 2H) | |
| 312 | ¹H NMR (400 MHz, DMSO-d6) δ13.57 (s, 1H), 11.05 (s, 1H), 10.03 (s, | A |
| | 1H), 9.01 (d, J = 8.4 Hz, 1H), 8.84 (s, 1H), 8.67-8.60 (m, 2H), 7.98 | |
| | (dd, J = 2.4, 9.2 Hz, 1H), 7.48 (d, J = 8.4 Hz, 1H), 6.97-6.83 (m, 3H), | |
| | 6.64 (d, J = 8.8 Hz, 1H), 5.31-5.23 (m, 1H), 4.71 (s, 2H), 4.59 (s, 1H), | |
| | 3.59 (d, J = 11.2 Hz, 2H), 3.40 (s, 4H), 3.25-2.95 (m, 5H), 2.89 (s, 2H), | |
| | 2.73-2.56 (m, 5H), 2.50 (s, 3H), 2.02-1.95 (m, 1H), 1.89 (dd, J = 1.6, | |
| | 8.0 Hz, 2H), 1.45 (d, J = 6.8 Hz, 6H), 1.42-1.27 (m, 2H) | |
| 313 | ¹H NMR (400 MHz, DMSO-d6) δ11.06 (s, 1H), 9.68 (s, 1H), 8.57 (s, | A |
| | 1H), 8.41 (s, 1H), 8.02 (d, J = 2.8 Hz, 1H), 7.93 (dd, J = 6.0, 7.2 Hz, | |
| | 1H), 7.69 (s, 1H), 7.52 (dd, J = 2.4, 10.0 Hz, 1H), 7.44 (dd, J = 2.8, 9.2 | |
| | Hz, 1H), 7.02-6.86 (m, 4H), 6.63 (dd, J = 1.6, 8.8 Hz, 1H), 5.26 (dd, J = | |
| | 5.2, 12.8 Hz, 1H), 4.64-4.51 (m, 1H), 4.26 (d, J = 18.4 Hz, 1H), 3.89 | |
| | (d, J = 18.0 Hz, 1H), 3.56 (d, J = 12.0 Hz, 2H), 3.15-3.10 (m, 4H), | |
| | 2.91-2.82 (m, 1H), 2.72-2.60 (m, 4H), 2.55-2.52 (m, 4H), 2.24 (d, J = 6.8 | |
| | Hz, 2H), 2.07 (s, 3H), 2.03-1.93 (m, 1H), 1.89-1.78 (m, 2H), 1.75- | |
| | 1.64 (m, 1H), 1.44 (d, J = 6.8 Hz, 6H), 1.35-1.22 (m, 2H) | |
| 314 | ¹H NMR (400 MHz, DMSO-d6) δ11.05 (s, 1H), 10.05 (s, 1H), 8.80 (s, | A |
| | 1H), 8.70 (d, J = 8.4 Hz, 1H), 8.30 (t, J = 7.2 Hz, 2H), 8.05 (d, J = 8.4 | |
| | Hz, 1H), 7.71-7.64 (m, 1H), 7.61-7.54 (m, 1H), 7.48 (d, J = 8.4 Hz, | |
| | 1H), 6.95-6.84 (m, 3H), 6.63 (dd, J = 1.2, 8.8 Hz, 1H), 5.26 (dd, J = | |
| | 5.2, 12.8 Hz, 1H), 4.64-4.54 (m, 3H), 3.56 d, J = 11.6 Hz, 2H), 2.86 (s, | |
| | 5H), 2.72-2.52 (m, 8H), 2.48 (s, 3H), 2.26 (d, J = 7.2 Hz, 2H), 2.02- | |
| | 1.94 (m, 1H), 1.83 (d, J = 12.0 Hz, 2H), 1.72-1.61 (m, 1H), 1.45 (d, J = | |
| | 6.8 Hz, 6H), 1.28 (d, J = 10.4 Hz, 2H) | |
| 315 | ¹H NMR (400 MHz, DMSO-d6) δ11.05 (s, 1H), 9.98 (s, 1H), 8.85 (s, | A |
| | 1H), 8.72 (d, J = 2.4 Hz, 1H), 8.61 (d, J = 8.4 Hz, 1H), 8.52 (d, J = 2.4 | |
| | Hz, 1H), 8.14-8.08 (m, 2H), 7.46 (d, J = 8.4 Hz, 1H), 6.95-6.82 (m, | |
| | 3H), 6.63 (dd, J = 1.2, 8.4 Hz, 1H), 5.25 (d, J = 12.8 Hz, 1H), 4.64- | |
| | 4.55 (m, 1H), 4.53 (s, 2H), 3.56 (d, J = 12.0 Hz, 2H), 2.93-2.80 (m, | |
| | 5H), 2.72-2.51 (m, 8H), 2.46 (s, 3H), 2.26 (d, J = 7.2 Hz, 2H), 2.02- | |
| | 1.93 (m, 1H), 1.83 (d, J = 11.6 Hz, 2H), 1.73-1.60 (m, 1H), 1.45 (d, J = | |
| | 6.8 Hz, 6H), 1.28 (d, J = 9.6 Hz, 2H) | |
| 316 | ¹H NMR (400 MHz, DMSO-d6) δ11.08 (s, 1H), 9.56 (s, 1H), 8.96 (s, | A |
| | 1H), 8.44 (s, 1H), 8.40 (s, 1H), 8.36 (s, 1H), 8.16 (s, 1H), 8.08 (s, 1H), | |
| | 8.00 (d, J = 2.8 Hz, 1H), 7.44 (dd, J = 2.8, 9.2 Hz, 1H), 6.96-6.84 (m, | |
| | 3H), 6.60 (d, J = 8.8 Hz, 1H), 5.28 (dd, J = 5.2, 12.8 Hz, 1H), 4.56 (td, | |
| | J = 6.8, 13.6 Hz, 1H), 4.08-4.04 (m, 1H), 4.00 (s, 3H), 3.96-3.88 (m, | |
| | 1H), 3.56-3.52 (m, 2H), 3.12 (s, 4H), 2.96-2.84 (m, 1H), 2.72-2.52 | |
| | (m, 8H), 2.40 (t, J = 6.0 Hz, 2H), 2.00-1.92 (m, 1H), 1.80 (d, J = 11.6 | |
| | Hz, 2H), 1.76-1.68 (m, 1H), 1.44 (d, J = 6.8 Hz, 9H), 1.36-1.24 (m, | |
| | 2H), 0.84-0.76 (m, 1H), 0.72-0.64 (m, 2H), 0.64-0.56 (m, 1H) | |
| 317 | ¹H NMR (400 MHz, DMSO-d6) δ11.16-10.96 (m, 1H), 9.64 (s, 1H), | A |
| | 8.96 (s, 1H), 8.44 (s, 1H), 8.40 (s, 1H), 8.36 (s, 1H), 8.28 (s, 1H), 7.44 | |
| | (d, J = 8.6 Hz, 1H), 6.96-6.88 (m, 2H), 6.80 (d, J = 8.8 Hz, 1H), 6.64 | |
| | (dd, J = 1.6, 8.8 Hz, 1H), 5.28 (dd, J = 5.2, 12.8 Hz, 1H), 4.56 (quin, J = | |
| | 6.8 Hz, 1H), 4.08-4.00 (m, 1H), 3.96 (s, 3H), 3.92-3.88 (m, 1H), 3.56 | |
| | (d, J = 11.6 Hz, 2H), 2.94-2.80 (m, 5H), 2.76-2.52 (m, 8H), 2.48 (s, | |
| | 3H), 2.44-2.36 (m, 2H), 2.00-1.96 (m, 1H), 1.80 (d, J = 12.0 Hz, 2H), | |
| | 1.76-1.68 (m, 1H), 1.44 (d, J = 6.8 Hz, 9H), 1.36-1.28 (m, 2H), 0.88- | |
| | 0.80 (m, 1H), 0.80-0.72 (m, 2H), 0.68-0.60 (m, 1H) | |
| 318 | ¹H NMR (400 MHz, DMSO-d6) δ11.06 (s, 1H), 9.90 (s, 1H), 9.28 (s, | A |
| | 1H), 9.04 (s, 1H), 8.89 (s, 1H), 8.39 (s, 1H), 8.25 (s, 1H), 7.54 (d, J = | |
| | 8.4 Hz, 1H), 6.96-6.88 (m, 3H), 6.65 (d, J = 8.4 Hz, 1H), 5.26 (dd, J = | |
| | 5.4, 12.8 Hz, 1H), 4.63-4.54 (m, 1H), 3.99 (s, 3H), 3.93 (d, J = 2.4 Hz, | |
| | 2H), 3.59 (d, J = 12.4 Hz, 4H), 3.43 (d, J = 4.0 Hz, 6H), 3.33 (s, 2H), | |
| | 2.92-2.82 (m, 2H), 2.71-2.56 (m, 6H), 2.01-1.83 (m, 4H), 1.45 (d, J = | |
| | 6.8 Hz, 6H), 1.41-1.26 (m, 2H) | |
| 319 | ¹H NMR (400 MHz, DMSO-d6) δ 11.06 (s, 1H), 9.65 (s, 1H), 8.46 (s, | A |
| | 1H), 8.35 (s, 1H), 8.18 (s, 1H), 8.15 (s, 1H), 7.50 (dd, J = 2.4, 8.8 Hz, | |
| | 1H), 7.46 (d, J = 8.8 Hz, 1H), 7.08 (dd, J = 2.4, 10.4 Hz, 1H), 6.92 (d, J = | |
| | 8.8 Hz, 1H), 6.90-6.84 (m, 2H), 6.63 (d, J = 8.8 Hz, 1H), 5.26 (dd, J = | |
| | 5.2, 12.8 Hz, 1H), 4.65-4.53 (m, 1H), 4.09-3.95 (m, 2H), 3.85 (s, | |
| | 3H), 3.56 (d, J = 11.2 Hz, 4H), 2.92 (d, J = 5.6 Hz, 1H), 2.84 (s, 4H), | |
| | 2.73-2.60 (m, 4H), 2.59 (s, 1H), 2.54 (s, 1H), 2.45 (s, 3H), 2.26 (d, J = | |
| | 6.8 Hz, 2H), 2.17 (s, 3H), 1.98 (dd, J = 5.2, 10.8 Hz, 1H), 1.83 (d, J = | |
| | 12.0 Hz, 2H), 1.67 (s, 1H), 1.45 (d, J = 6.8 Hz, 6H), 1.34-1.22 (m, 2H) | |
| 320 | ¹H NMR (400 MHz, DMSO-d6) δ 11.05 (s, 1H), 9.98 (s, 1H), 8.84 (s, | A |
| | 1H), 8.68-8.53 (m, 2H), 8.24-8.20 (m, 1H), 8.19 (s, 1H), 8.16 (d, J = | |
| | 8.8 Hz, 1H), 8.10 (s, 1H), 7.46 (d, J = 8.8 Hz, 1H), 7.34-7.25 (m, 1H), | |
| | 6.98-6.81 (m, 3H), 6.69-6.58 (m, 1H), 5.34-5.20 (m, 1H), 4.64-4.50 | |
| | (m, 3H), 3.61-3.52 (m, 4H), 2.91-2.81 (m, 5H), 2.71-2.60 (m, 4H), | |
| | 2.54 (d, J = 2.4 Hz, 2H), 2.47 (s, 3H), 2.30-2.22 (m, 2H), 2.02-1.94 | |
| | (m, 1H), 1.87-1.78 (m, 2H), 1.72-1.62 (m, 1H), 1.45 (d, J = 6.8 Hz, | |
| | 6H), 1.34-1.23 (m, 2H) | |
| 321 | ¹H NMR (400 MHz, DMSO-d6) δ11.13-10.99 (m, 1H), 10.02 (s, 1H), | A |
| | 8.83 (s, 1H), 8.62 (d, J = 14.4 Hz, 1H), 8.23-8.18 (m, 1H), 7.89 (s, 1H), | |
| | 7.56 (dd, J = 2.4, 10.0 Hz, 1H), 7.49 (d, J = 8.8 Hz, 1H), 7.01 (dt, J = | |
| | 2.8, 7.6 Hz, 1H), 6.94-6.85 (m, 3H), 6.66-6.61 (m, 1H), 5.27 (dd, J = | |
| | 5.6, 12.8 Hz, 1H), 4.64-4.54 (m, 1H), 4.40 (s, 2H), 3.55 (s, 4H), 2.86 | |
| | (s, 4H), 2.67-2.54 (m, 7H), 2.48 (s, 3H), 2.26 (d, J = 6.8 Hz, 2H), 1.99 | |
| | (dd, J = 4.8, 10.4 Hz, 1H), 1.83 (d, J = 11.2 Hz, 2H), 1.73-1.62 (m, | |
| | 1H), 1.45 (d, J = 6.8 Hz, 6H), 1.33-1.23 (m, 2H) | |
| 322 | ¹H NMR (400 MHz, DMSO-d6) δ 11.06 (s, 1H), 9.67 (s, 1H), 8.99 (s, | A |
| | 1H), 8.47 (s, 1H), 8.39 (s, 2H), 8.32 (s, 1H), 8.15 (s, 1H), 7.46 (d, J = | |
| | 8.8 Hz, 1H), 6.89 (t, J = 8.0 Hz, 3H), 6.63 (dd, J = 1.6, 8.8 Hz, 1H), 5.26 | |
| | (dd, J = 5.2, 12.8 Hz, 1H), 4.59 (td, J = 6.8, 13.6 Hz, 1H), 4.06 (s, 1H), | |
| | 4.02-3.96 (m, 4H), 3.56 (d, J = 11.2 Hz, 4H), 2.85 (s, 5H), 2.63 (s, 3H), | |
| | 2.60-2.53 (m, 3H), 2.45 (s, 3H), 2.27 (d, J = 6.8 Hz, 2H), 2.18 (s, 3H), | |
| | 2.03-1.94 (m, 1H), 1.83 (d, J = 11.8 Hz, 2H), 1.73-1.60 (m, 1H), 1.45 | |
| | (d, J = 6.8 Hz, 6H), 1.35-1.22 (m, 2H) | |
| 323 | ¹H NMR (400 MHz, DMSO-d6) δ 11.07 (s, 1H), 9.69 (s, 1H), 8.58 (s, | A |
| | 1H), 8.30 (s, 1H), 8.22 (s, 1H), 8.02-7.94 (m, 1H), 7.76 (s, 1H), 7.53 | |
| | (dd, J = 2.8, 10.0 Hz, 1H), 7.46 (d, J = 8.4 Hz, 1H), 7.01-6.85 (m, 3H), | |
| | 6.80 (d, J = 8.4 Hz, 1H), 6.62 (dd, J = 1.6, 8.4 Hz, 1H), 5.26 (dd, J = 5.2, | |
| | 12.8 Hz, 1H), 4.58 (td, J = 6.8, 13.6 Hz, 1H), 4.32 (d, J = 18.4 Hz, 1H), | |
| | 3.88 (d, J = 18.4 Hz, 1H), 3.54 (d, J = 11.2 Hz, 3H), 2.92-2.78 (m, 5H), | |
| | 2.73-2.55 (m, 6H), 2.45 (s, 3H), 2.41 (d, J = 6.4 Hz, 2H), 2.01-1.92 | |
| | (m, 1H), 1.79 (d, J = 11.6 Hz, 2H), 1.55-1.24 (m, 12H), 1.01-0.68 (m, | |
| | 4H) | |
| 324 | ¹H NMR (400 MHz, DMSO-d6) δ11.04 (s, 1H), 9.56 (s, 1H), 9.00 (s, | A |
| | 1H), 8.44 (s, 1H), 8.40 (s, 1H), 8.34 (s, 1H), 8.16 (s, 1H), 8.08 (s, 1H), | |
| | 8.00 (d, J = 2.8 Hz, 1H), 7.44 (dd, J = 2.8, 9.2 Hz, 1H), 6.96-6.84 (m, | |
| | 3H), 6.64 (dd, J = 1.6, 8.8 Hz, 1H), 5.28 (dd, J = 5.2, 12.8 Hz, 1H), 4.56 | |
| | (td, J = 6.8, 13.6 Hz, 1H), 4.08-3.88 (m, 5H), 3.56 (d, J = 11.6 Hz, 2H), | |
| | 3.12 (s, 4H), 2.96-2.84 (m, 1H), 2.72-2.60 (m, 4H), 2.52 (d, J = 4.0 | |
| | Hz, 4H), 2.24 (d, J = 6.8 Hz, 2H), 2.04-1.96 (m, 1H), 1.84 (d, J = 12.0 | |
| | Hz, 2H), 1.76-1.60 (m, 2H), 1.44 (d, J = 6.8 Hz, 6H), 1.36-1.20 (m, | |
| | 2H), 0.84-0.76 (m, 1H), 0.72-0.64 (m, 2H), 0.60-0.56 (m, 1H) | |
| 325 | ¹H NMR (400 MHz, DMSO-d6) δ 11.06 (s, 1H), 9.72 (s, 1H), 8.79 (s, | A |
| | 1H), 8.56 (s, 1H), 8.01-7.86 (m, 1H), 7.66 (s, 1H), 7.57-7.41 (m, 2H), | |
| | 7.07-6.71 (m, 4H), 6.70-6.55 (m, 1H), 5.26 (br dd, J = 5.2, 12.4 Hz, | |
| | 1H), 4.59 (quin, J = 6.8 Hz, 1H), 4.11 (d, J = 18.0 Hz, 1H), 3.80 (d, J = | |
| | 18.0 Hz, 1H), 3.56 (d, J = 11.6 Hz, 2H), 2.97-2.77 (m, 5H), 2.73-2.51 | |
| | (m, 8H), 2.47 (s, 4H), 2.26 (d, J = 6.8 Hz, 2H), 2.03-1.93 (m, 1H), 1.83 | |
| | (d, J = 11.6 Hz, 2H), 1.67 (d, J = 4.0 Hz, 1H), 1.45 (d, J = 6.8 Hz, 6H), | |
| | 1.28 (q, J = 10.8 Hz, 2H), 1.17 (d, J = 6.8 Hz, 3H), 1.13-1.05 (m, 3H) | |
| 326 | ¹H NMR (400 MHz, DMSO-d6) δ 11.06 (s, 1H), 9.69 (s, 1H), 8.57 (s, | A |
| | 1H), 8.30 (s, 1H), 7.98 (dd, J = 6.4, 7.2 Hz, 1H), 7.75 (s, 1H), 7.52 (dd, J = | |
| | 2.4, 10.0 Hz, 1H), 7.46 (d, J = 8.4 Hz, 1H), 7.02-6.85 (m, 3H), 6.80 | |
| | (d, J = 8.4 Hz, 1H), 6.63 (dd, J = 1.6, 8.4 Hz, 1H), 5.27 (dd, J = 5.2, 12.8 | |
| | Hz, 1H), 4.59 (quin, J = 6.8 Hz, 1H), 4.31 (d, J = 18.0 Hz, 1H), 3.88 (d, | |
| | J = 18.0 Hz, 1H), 3.56 (d, J = 11.6 Hz, 2H), 2.94-2.78 (m, 5H), 2.74- | |
| | 2.52 (m, 7H), 2.45 (s, 3H), 2.25 (d, J = 7.2 Hz, 2H), 2.03-1.91 (m, 1H), | |
| | 1.82 (d, J = 11.6 Hz, 2H), 1.73-1.60 (m, 1H), 1.45 (d, J = 6.8 Hz, 6H), | |
| | 1.40-1.22 (m, 3H), 0.99-0.67 (m, 4H) | |
| 327 | ¹H NMR (400 MHz, DMSO-d6) δ 11.06 (s, 1H), 8.64 (s, 1H), 8.34 (dd, | A |
| | J = 6.0, 7.2 Hz, 1H), 8.19 (s, 1H), 7.76 (s, 1H), 7.55-7.47 (m, 2H), 6.96- | |
| | 6.87 (m, 3H), 6.82 (d, J = 12.0 Hz, 2H), 6.63 (dd, J = 1.6, 8.8 Hz, 1H), | |
| | 6.42 (d, J = 8.4 Hz, 1H), 5.27 (dd, J = 5.2, 12.8 Hz, 1H), 4.63-4.54 (m, | |
| | 1H), 4.35 (s, 2H), 3.56 (d, J = 12.0 Hz, 2H), 3.32 (d, J = 9.6 Hz, 4H), | |
| | 2.88 (s, 1H), 2.72-2.52 (m, 8H), 2.30 (s, 2H), 2.02-1.93 (m, 1H), 1.82 | |
| | (d, J = 12.0 Hz, 2H), 1.75-1.63 (m, 1H), 1.45 (d, J = 6.8 Hz, 6H), 1.29 | |
| | (d, J = 9.6 Hz, 2H) | |
| 328 | ¹H NMR (400 MHz, DMSO-d6) δ 11.06 (s, 1H), 9.93 (s, 1H), 8.79 (s, | A |
| | 1H), 8.65 (d, J = 8.4 Hz, 1H), 8.44 (dd, J = 6.0, 7.2 Hz, 1H), 7.81 (s, | |
| | 1H), 7.70 (d, J = 8.4 Hz, 1H), 7.51 (dd, J = 2.4, 10.0 Hz, 1H), 7.46 (d, J = | |
| | 8.8 Hz, 1H), 7.15 (d, J = 8.8 Hz, 1H), 6.96 (dt, J = 2.4, 7.6 Hz, 1H), | |
| | 6.84 (d, J = 8.8 Hz, 1H), 6.78 (s, 1H), 6.65 (dd, J = 1.6, 8.8 Hz, 1H), | |
| | 5.31 (dd, J = 5.6, 12.4 Hz, 1H), 4.58-4.49 (m, 1H), 4.38 (s, 2H), 2.84 | |
| | (s, 5H), 2.73 (dd, J = 4.4, 12.8 Hz, 2H), 2.66-2.52 (m, 8H), 2.45 (s, | |
| | 3H), 2.26 (d, J = 6.8 Hz, 2H), 1.99 (d, J = 5.6 Hz, 1H), 1.82 (d, J = 11.6 | |
| | Hz, 2H), 1.71-1.60 (m, 1H), 1.41 (d, J = 6.8 Hz, 6H), 1.26 (d, J = 12.4 | |
| | Hz, 2H) | |
| 329 | ¹H NMR (400 MHz, DMSO-d6) δ 11.06 (s, 1H), 10.08 (s, 1H), 8.84 (s, | A |
| | 1H), 8.51 (d, J = 8.4 Hz, 1H), 8.46 (dd, J = 6.0, 7.2 Hz, 1H), 7.83 (s, | |
| | 1H), 7.73 (d, J = 8.8 Hz, 1H), 7.60 (d, J = 8.8 Hz, 1H), 7.52 (dd, J = 2.8, | |
| | 10.0 Hz, 1H), 7.05 (d, J = 8.8 Hz, 1H), 6.97-6.85 (m, 3H), 6.63 (dd, J = | |
| | 1.6, 8.8 Hz, 1H), 5.27 (dd, J = 5.2, 12.8 Hz, 1H), 4.63-4.54 (m, 1H), | |
| | 4.40 (s, 2H), 3.55 (d, J = 11.6 Hz, 2H), 3.34 (s, 4H), 3.03 (s, 3H), 2.95- | |
| | 2.83 (m, 4H), 2.81 (s, 3H), 2.65-2.58 (m, 3H), 2.47 (dd, J = 1.6, 3.2 | |
| | Hz, 2H), 2.23 (d, J = 6.0 Hz, 2H), 2.02-1.93 (m, 1H), 1.81 (d, J = 11.6 | |
| | Hz, 2H), 1.66 (s, 1H), 1.45 (d, J = 6.8 Hz, 6H), 1.33-1.20 (m, 2H) | |
| 330 | ¹H NMR (400 MHz, DMSO-d6) δ 11.05 (s, 1H), 9.94 (s, 1H), 8.80 (s, | A |
| | 1H), 8.66 (d, J = 8.4 Hz, 1H), 8.48-8.42 (m, 1H), 7.82 (s, 1H), 7.71 (d, | |
| | J = 8.8 Hz, 1H), 7.61 (s, 1H), 7.53 (dd, J = 2.4, 10.0 Hz, 1H), 7.48 (d, J = | |
| | 8.4 Hz, 1H), 7.37 (d, J = 2.0 Hz, 1H), 6.97 (dt, J = 2.4, 7.2 Hz, 1H), | |
| | 6.85 (d, J = 8.8 Hz, 1H), 5.30 (dd, J = 5.6, 12.4 Hz, 1H), 4.66-4.58 (m, | |
| | 1H), 4.39 (s, 2H), 3.63-3.56 (m, 2H), 3.00-2.89 (m, 2H), 2.86 (s, 5H), | |
| | 2.69-2.63 (m, 4H), 2.57-2.55 (m, 2H), 2.47 (s, 3H), 2.27 (d, J = 7.2 | |
| | Hz, 2H), 2.07-2.01 (m, 1H), 1.84 (d, J = 11.2 Hz, 2H), 1.73-1.65 (m, | |
| | 1H), 1.47 (d, J = 6.8 Hz, 6H), 1.35-1.24 (m, 2H) | |
| 331 | ¹H NMR (400 MHz, DMSO-d6) δ 11.05 (s, 1H), 9.99 (s, 1H), 8.80 (s, | A |
| | 1H), 8.71 (d, J = 8.5 Hz, 1H), 8.41 (dd, J = 5.9, 7.3 Hz, 1H), 8.18 (s, | |
| | 1H), 7.80 (s, 1H), 7.68 (d, J = 8.5 Hz, 1H), 7.55 (d, J = 8.8 Hz, 1H), 7.51 | |
| | (dd, J = 2.5, 10.0 Hz, 1H), 6.98-6.89 (m, 3H), 6.88 (d, J = 1.8 Hz, 1H), | |
| | 6.62 (dd, J = 1.8, 8.8 Hz, 1H), 5.25 (dd, J = 5.3, 12.7 Hz, 1H), 4.62- | |
| | 4.53 (m, 1H), 4.36 (s, 2H), 3.65-3.50 (m, 6H), 2.97 (s, 4H), 2.89-2.82 | |
| | (m, 1H), 2.68-2.56 (m, 6H), 2.33 (s, 6H), 2.25 (d, J = 7.0 Hz, 2H), 2.01- | |
| | 1.93 (m, 1H), 1.82 (d, J = 10.9 Hz, 2H), 1.71-1.61 (m, 1H), 1.44 (d, | |
| | J = 6.8 Hz, 6H), 1.33-1.21 (m, 2H) | |
| 332 | ¹H NMR (400 MHz, DMSO-d6) δ 11.10 (s, 1H), 9.95 (s, 1H), 8.81 (s, | A |
| | 1H), 8.67 (d, J = 8.4 Hz, 1H), 8.45 (dd, J = 6.0, 7.2 Hz, 1H), 7.83 (d, J = | |
| | 4.4 Hz, 2H), 7.71 (d, J = 8.8 Hz, 1H), 7.53 (dd, J = 2.8, 10.0 Hz, 1H), | |
| | 7.48 (d, J = 8.4 Hz, 1H), 7.00-6.94 (m, 1H), 6.85 (d, J = 8.4 Hz, 1H), | |
| | 6.74 (s, 1H), 5.29 (dd, J = 5.2, 12.8 Hz, 1H), 4.63-4.55 (m, 1H), 4.39 | |
| | (s, 2H), 4.24 (d, J = 11.6 Hz, 2H), 3.33-3.28 (m, 2H), 2.87 (s, 5H), 2.79- | |
| | 2.58 (m, 7H), 2.47 (s, 3H), 2.33-2.25 (m, 1H), 2.03 (s, 1H), 1.79 (d, J = | |
| | 10.8 Hz, 3H), 1.46 (d, J = 6.8 Hz, 6H), 1.22-1.10 (m, 2H) | |
| 333 | ¹H NMR (400 MHz, DMSO-d6) δ10.37 (s, 1H), 9.94 (s, 1H), 8.79 (s, | A |
| | 1H), 8.68-8.62 (m, 1H), 8.47-8.40 (m, 1H), 7.81 (d, J = 4.0 Hz, 1H), | |
| | 7.73-7.66 (m, 1H), 7.55-7.44 (m, 2H), 7.27-7.20 (m, 1H), 7.03-6.90 | |
| | (m, 3H), 6.84 (d, J = 8.0 Hz, 1H), 4.38 (s, 2H), 3.76 (d, J = 9.6 Hz, 2H), | |
| | 3.65-3.50 (m, 3H), 2.84 (s, 5H), 2.79-2.66 (m, 6H), 2.46 (s, 3H), 2.23 | |
| | (d, J = 1.6 Hz, 2H), 1.85-1.71 (m, 3H), 1.25-1.11 (m, 2H) | |
| 334 | ¹H NMR (400 MHz, DMSO-d6) δ 11.18 (s, 1H), 10.18-9.76 (m, 2H), | A |
| | 8.88 (s, 1H), 8.77-8.64 (m, 2H), 8.27 (s, 1H), 7.95 (dd, J = 2.4, 8.6 Hz, | |
| | 1H), 7.77 (d, J = 8.8 Hz, 1H), 7.54 (d, J = 8.8 Hz, 1H), 7.48-7.30 (m, | |
| | 2H), 6.94 (d, J = 8.8 Hz, 1H), 6.79-6.64 (m, 1H), 5.33 (dd, J = 4.8, 13.2 | |
| | Hz, 1H), 4.58 (quin, J = 7.2 Hz, 1H), 4.41 (s, 2H), 3.79-3.41 (m, 5H), | |
| | 3.33-3.06 (m, 8H), 3.00-2.58 (m, 2H), 2.47-2.39 (m, 2H), 2.37-2.11 | |
| | (m, 3H), 2.10-1.70 (m, 6H), 1.46 (d, J = 6.8 Hz, 6H) | |
| 335 | ¹H NMR (400 MHz, DMSO-d6) δ 11.10 (s, 1H), 9.94 (s, 1H), 8.79 (s, | A |
| | 1H), 8.66 (d, J = 8.4 Hz, 1H), 8.44 (dd, J = 6.0, 7.0 Hz, 1H), 8.16 (s, | |
| | 1H), 7.81 (s, 1H), 7.70 (d, J = 8.8 Hz, 1H), 7.59-7.42 (m, 3H), 6.96 (dt, | |
| | J = 2.4, 7.2 Hz, 1H), 6.84 (d, J = 8.8 Hz, 1H), 5.31 (dd, J = 5.6, 12.8 Hz, | |
| | 1H), 4.61 (td, J = 6.8, 13.6 Hz, 1H), 4.38 (s, 2H), 4.12 (d, J = 12.4 Hz, | |
| | 2H), 2.99-2.72 (m, 9H), 2.70-2.58 (m, 3H), 2.46 (s, 3H), 2.29-2.20 | |
| | (m, 2H), 2.16-2.06 (m, 1H), 1.81 (d, J = 10.0 Hz, 3H), 1.51 (dd, J = | |
| | 1.6, 6.8 Hz, 6H), 1.27-1.10 (m, 2H) | |
| 336 | ¹H NMR (400 MHz, DMSO-d6) δ 11.11 (s, 1H), 9.94 (s, 1H), 8.80 (s, | A |
| | 1H), 8.66 (d, J = 8.4 Hz, 1H), 8.44 (dd, J = 6.0, 7.2 Hz, 1H), 7.82 (s, | |
| | 1H), 7.70 (d, J = 8.8 Hz, 1H), 7.52 (dd, J = 2.8, 10.0 Hz, 1H), 7.46 (d, J = | |
| | 8.8 Hz, 1H), 6.96 (dt, J = 2.8, 7.6 Hz, 1H), 6.84 (dd, J = 3.2, 8.4 Hz, | |
| | 2H), 6.73 (t, J = 8.0 Hz, 1H), 5.33 (d, J = 12.8 Hz, 1H), 4.38 (s, 2H), | |
| | 3.47 (d, J = 1.6 Hz, 3H), 3.34 (s, 4H), 3.24 (d, J = 11.2 Hz, 3H), 2.87 (s, | |
| | 4H), 2.68-2.60 (m, 5H), 2.46 (s, 3H), 2.37 (s, 1H), 2.06-1.95 (m, 1H), | |
| | 1.83 (d, J = 11.2 Hz, 2H), 1.69 (s, 1H), 1.32 (d, J = 10.4 Hz, 2H) | |
| 337 | ¹H NMR (400 MHz, DMSO-d6) δ 11.11 (s, 1H), 9.94 (s, 1H), 8.80 (s, | A |
| | 1H), 8.66 (d, J = 8.4 Hz, 1H), 8.50-8.40 (m, 1H), 7.99 (s, 1H), 7.82 (s, | |
| | 1H), 7.71 (s, 1H), 7.58-7.40 (m, 2H), 6.96 (dt, J = 2.4, 7.6 Hz, 1H), | |
| | 6.84 (d, J = 8.8 Hz, 1H), 5.30 (dd, J = 5.2, 12.8 Hz, 1H), 4.66-4.50 (m, | |
| | 3H), 4.38 (s, 2H), 2.98-2.77 (m, 7H), 2.68-2.56 (m, 4H), 2.49-2.42 | |
| | (m, 5H), 2.21 (d, J = 6.4 Hz, 2H), 2.06 (dd, J = 5.2, 10.4 Hz, 1H), 1.78 | |
| | (d, J = 12.4 Hz, 3H), 1.49 (d, J = 6.8 Hz, 6H), 1.14-0.98 (m, 2H) | |
| 338 | ¹H NMR (400 MHz, DMSO-d6) δ 11.08 (s, 1H), 9.94 (s, 1H), 8.80 (s, | A |
| | 1H), 8.71-8.62 (m, 1H), 8.49-8.41 (m, 1H), 8.13 (s, 1H), 7.82 (s, 1H), | |
| | 7.71 (d, J = 8.4 Hz, 1H), 7.57-7.43 (m, 2H), 7.33-7.24 (m, 1H), 7.00- | |
| | 6.92 (m, 1H), 6.89-6.81 (m, 1H), 6.39 (s, 1H), 5.35-5.23 (m, 1H), | |
| | 4.67-4.54 (m, 1H), 4.38 (s, 2H), 4.16 (d, J = 12.4 Hz, 2H), 2.95-2.73 | |
| | (m, 8H), 2.67-2.55 (m, 6H), 2.46 (s, 3H), 2.27 (d, J = 4.0 Hz, 1H), 2.07- | |
| | 1.98 (m, 1H), 1.79 (d, J = 11.2 Hz, 3H), 1.50 (d, J = 6.8 Hz, 6H), 1.23- | |
| | 1.11 (m, 2H) | |
| 339 | ¹H NMR (400 MHz, DMSO-d6) δ 10.52 (s, 1H), 9.94 (s, 1H), 8.79 (s, | A |
| | 1H), 8.65 (d, J = 8.4 Hz, 1H), 8.48-8.39 (m, 1H), 7.81 (s, 1H), 7.70 (d, | |
| | J = 8.4 Hz, 1H), 7.64 (d, J = 8.0 Hz, 1H), 7.56 (d, J = 2.0 Hz, 1H), 7.52 | |
| | (dd, J = 2.4, 10.0 Hz, 1H), 7.46 (d, J = 8.8 Hz, 1H), 7.39 (dd, J = 2.0, 8.0 | |
| | Hz, 1H), 6.96 (dt, J = 2.4, 7.6 Hz, 1H), 6.84 (d, J = 8.4 Hz, 1H), 4.51- | |
| | 4.42 (m, 1H), 4.38 (s, 2H), 3.82-3.72 (m, 1H), 3.67-3.54 (m, 2H), | |
| | 3.14-3.00 (m, 1H), 2.84 (s, 4H), 2.81-2.72 (m, 3H), 2.56-2.52 (m, | |
| | 5H), 2.45 (s, 3H), 2.25 (s, 1H), 1.92-1.79 (m, 2H), 1.74-1.63 (m, 1H), | |
| | 1.18-1.04 (m, 2H) | |
| 340 | ¹H NMR (400 MHz, DMSO-d6) δ 10.82 (s, 1H), 9.95 (s, 1H), 8.80 (s, | A |
| | 1H), 8.67 (d, J = 8.4 Hz, 1H), 8.45 (dd, J = 6.0, 7.6 Hz, 1H), 8.15 (s, | |
| | 1H), 7.82 (s, 1H), 7.71 (d, J = 8.4 Hz, 1H), 7.57-7.42 (m, 2H), 7.08- | |
| | 6.92 (m, 4H), 6.85 (d, J = 8.4 Hz, 1H), 6.55 (s, 1H), 4.39 (s, 2H), 3.80 | |
| | (dd, J = 4.8, 11.6 Hz, 1H), 3.37-3.35 (m, 2H), 2.86 (s, 4H), 2.73-2.55 | |
| | (m, 6H), 2.47 (s, 3H), 2.33-2.13 (m, 3H), 2.00 (td, J = 4.4, 8.8 Hz, 1H), | |
| | 1.83 (d, J = 11.6 Hz, 2H), 1.69 (d, J = 6.8 Hz, 1H), 1.40-1.20 (m, 2H) | |
| 341 | ¹H NMR (400 MHz, DMSO-d6) δ 10.99-10.80 (m, 1H), 9.94 (s, 1H), | A |
| | 8.79 (s, 1H), 8.66 (d, J = 8.4 Hz, 1H), 8.44 (t, J = 6.8 Hz, 1H), 8.14 (s, | |
| | 1H), 7.82 (s, 1H), 7.71 (d, J = 8.8 Hz, 1H), 7.58-7.43 (m, 2H), 7.40- | |
| | 7.11 (m, 3H), 6.97 (dt, J = 2.4, 7.6 Hz, 1H), 6.84 (d, J = 8.4 Hz, 1H), | |
| | 4.58-4.46 (m, 1H), 4.39 (s, 2H), 3.97 (dd, J = 4.8, 12.0 Hz, 1H), 3.44 | |
| | (s, 2H), 3.13-3.02 (m, 1H), 2.89-2.76 (m, 5H), 2.76-2.64 (m, 2H), | |
| | 2.56 (d, J = 4.0 Hz, 2H), 2.46 (s, 3H), 2.32-2.19 (m, 3H), 2.06 (td, J = | |
| | 4.0, 8.4 Hz, 1H), 1.93-1.80 (m, 2H), 1.71 (d, J = 10.8 Hz, 1H), 1.19- | |
| | 0.97 (m, 2H) | |
| 342 | ¹H NMR (400 MHz, DMSO-d6) δ 10.94 (s, 1H), 9.94 (s, 1H), 8.80 (s, | A |
| | 1H), 8.66 (d, J = 8.4 Hz, 1H), 8.45 (dd, J = 6.0, 7.6 Hz, 1H), 8.17 (s, | |
| | 1H), 7.82 (s, 1H), 7.71 (d, J = 8.4 Hz, 1H), 7.53 (dd, J = 2.8, 10.0 Hz, | |
| | 1H), 7.49-7.36 (m, 2H), 7.28-7.16 (m, 2H), 6.97 (dt, J = 2.8, 7.6 Hz, | |
| | 1H), 6.84 (d, J = 8.4 Hz, 1H), 4.53-4.34 (m, 3H), 4.12 (dd, J = 4.8, 12.4 | |
| | Hz, 1H), 3.63-3.53 (m, 1H), 3.20-2.97 (m, 2H), 2.87-2.72 (m, 6H), | |
| | 2.58 (d, J = 3.6 Hz, 1H), 2.55-2.52 (m, 3H), 2.46 (s, 3H), 2.31-2.18 | |
| | (m, 3H), 2.09-1.97 (m, 1H), 1.95-1.61 (m, 3H), 1.20-1.03 (m, 2H) | |
| 343 | ¹H NMR (400 MHz, DMSO-d6) δ 10.53 (s, 1H), 9.94 (s, 1H), 8.79 (s, | A |
| | 1H), 8.66 (d, J = 8.4 Hz, 1H), 8.49-8.41 (m, 1H), 8.18 (s, 1H), 7.82 (s, | |
| | 1H), 7.71 (d, J = 8.4 Hz, 1H), 7.60-7.50 (m, 3H), 7.49-7.39 (m, 2H), | |
| | 6.97 (dt, J = 2.4, 7.6 Hz, 1H), 6.84 (d, J = 8.4 Hz, 1H), 4.56-4.31 (m, | |
| | 3H), 3.85-3.58 (m, 4H), 3.15-3.04 (m, 2H), 2.84 (s, 5H), 2.78-2.70 | |
| | (m, 3H), 2.46 (s, 3H), 2.25 (d, J = 5.2 Hz, 2H), 1.95-1.44 (m, 4H), | |
| | 1.22-1.05 (m, 2H) | |
| 344 | ¹H NMR (400 MHz, DMSO-d6) δ 11.12-10.98 (m, 1H), 9.93 (s, 1H), | A |
| | 8.78 (s, 1H), 8.64 (d, J = 8.4 Hz, 1H), 7.87 (dd, J = 4.4, 7.2 Hz, 1H), | |
| | 7.75 (s, 1H), 7.68 (d, J = 8.4 Hz, 1H), 7.47 (d, J = 8.8 Hz, 1H), 7.35- | |
| | 7.03 (m, 1H), 7.02-6.81 (m, 4H), 6.63 (dd, J = 1.6, 8.8 Hz, 1H), 5.26 | |
| | (d, J = 12.4 Hz, 1H), 4.65-4.53 (m, 1H), 4.37 (s, 2H), 3.56 (d, J = | |
| | 12.0Hz, 2H), 2.95-2.80 (m, 5H), 2.65-2.53 (m, 8H), 2.46 (s, 3H), 2.26 | |
| | (d, J = 7.2 Hz, 2H), 2.02-1.93 (m, 1H), 1.83 (d, J = 11.2 Hz, 2H), 1.71- | |
| | 1.63 (m, 1H), 1.45 (d, J = 6.8 Hz, 6H), 1.35-1.22 (m, 2H) | |
| 345 | ¹H NMR (400 MHz, DMSO-d6) δ 10.82 (s, 1H), 9.94 (s, 1H), 8.80 (s, | A |
| | 1H), 8.66 (d, J = 8.8 Hz, 1H), 8.49-8.40 (m, 1H), 7.82 (s, 1H), 7.70 (d, | |
| | J = 8.8 Hz, 1H), 7.52 (dd, J = 2.4, 10.1 Hz, 1H), 7.46 (d, J = 8.8 Hz, | |
| | 1H), 7.06 (t, J = 8.8 Hz, 1H), 6.96 (dt, J = 2.8, 7.5 Hz, 1H), 6.84 (d, J = | |
| | 8.4 Hz, 1H), 6.72 (s, 1H), 6.69 (s, 1H), 4.38 (s, 2H), 3.87 (dd, J = 4.8, | |
| | 12.3 Hz, 1H), 3.72 (d, J = 12.4 Hz, 2H), 2.85 (s, 4H), 2.77-2.63 (m, | |
| | 4H), 2.63-2.52 (m, 4H), 2.45 (s, 3H), 2.28 (s, 2H), 2.14 (dq, J = 4.0, | |
| | 12.7 Hz, 1H), 1.99-1.91 (m, 1H), 1.79 (d, J = 12.4 Hz, 2H), 1.73 (d, J = | |
| | 2.0 Hz, 1H), 1.26-1.12 (m, 2H) | |
| 346 | ¹H NMR (400 MHz, DMSO-d6) δ 10.56 (s, 1H), 9.96 (s, 1H), 9.33 (s, | A |
| | 1H), 8.82 (s, 1H), 8.68 (d, J = 8.8 Hz, 1H), 8.58 (s, 1H), 8.46 (dd, J = | |
| | 5.6, 7.6 Hz, 1H), 8.14 (s, 1H), 7.83 (s, 1H), 7.77-7.65 (m, 2H), 7.58- | |
| | 7.43 (m, 2H), 7.19 (d, J = 1.6 Hz, 1H), 6.97 (dt, J = 2.8, 7.6 Hz, 1H), | |
| | 6.86 (d, J = 8.4 Hz, 1H), 4.40 (s, 2H), 4.01-3.85 (m, 1H), 3.79-3.66 | |
| | (m, 1H), 3.53-3.40 (m, 2H), 3.06-2.84 (m, 7H), 2.77-2.66 (m, 2H), | |
| | 2.65-2.57 (m, 2H), 2.48 (s, 3H), 2.45-2.35 (m, 2H), 1.95 (d, J = 12.4 | |
| | Hz, 2H), 1.89-1.76 (m, 1H), 1.63-1.47 (m, 2H) | |
| 347 | ¹H NMR (400 MHz, DMSO-d6) δ 10.56-10.49 (m, 1H), 9.98-9.89 | A |
| | (m, 1H), 9.27 (s, 1H), 8.85-8.75 (m, 1H), 8.70 (s, 1H), 8.58 (d, J = 3.4 | |
| | Hz, 1H), 8.44 (dd, J = 6.2, 7.2 Hz, 1H), 7.87-7.77 (m, 1H), 7.75-7.66 | |
| | (m, 1H), 7.56-7.44 (m, 2H), 7.37 (dd, J = 1.7, 9.8 Hz, 1H), 7.15-7.05 | |
| | (m, 1H), 7.00 (s, 1H), 6.91-6.73 (m, 1H), 4.38 (s, 2H), 3.97-3.84 (m, | |
| | 1H), 3.75-3.64 (m, 1H), 3.54-3.37 (m, 3H), 3.05-2.96 (m, 1H), 2.92- | |
| | 2.79 (m, 6H), 2.79-2.64 (m, 2H), 2.57 (s, 3H), 2.47 (s, 3H), 2.36- | |
| | 2.34 (m, 1H), 1.94 (d, J = 10.5 Hz, 2H), 1.87-1.73 (m, 1H), 1.62-1.45 | |
| | (m, 2H) | |
| 348 | ¹H NMR (400 MHz, DMSO-d6) δ 11.08 (s, 1H), 9.90 (s, 1H), 8.76 (s, | A |
| | 1H), 8.55 (d, J = 8.8 Hz, 1H), 7.85 (s, 1H), 7.62 (dd, J = 4.8, 8.8 Hz, | |
| | 1H), 7.46 (d, J = 8.8 Hz, 1H), 7.33-7.24 (m, 1H), 6.92 (d, J = 8.4 Hz, | |
| | 1H), 6.88-6.81 (m, 2H), 6.75 (ddd, J = 3.2, 6.4, 8.4 Hz, 1H), 6.66 (dd, J = | |
| | 2.0, 8.4 Hz, 1H), 5.26 (dd, J = 5.2, 12.8 Hz, 1H), 4.37 (s, 2H), 3.56 (d, | |
| | J = 11.2 Hz, 3H), 2.92-2.80 (m, 6H), 2.70-2.53 (m, 7H), 2.46 (s, 3H), | |
| | 2.26 (d, J = 6.8 Hz, 2H), 2.02-1.92 (m, 1H), 1.84 (d, J = 11.6 Hz, 2H), | |
| | 1.73-1.62 (m, 1H), 1.29 (d, J = 10.0 Hz, 2H), 1.09-1.01 (m, 2H), | |
| | 0.91-0.84 (m, 2H) | |
| 349 | ¹H NMR (400 MHz, DMSO-d6) δ 11.11 (s, 1H), 9.90 (s, 1H), 8.76 (s, | A |
| | 1H), 8.55 (d, J = 8.8 Hz, 1H), 7.85 (s, 1H), 7.62 (dd, J = 4.4, 8.4 Hz, | |
| | 1H), 7.46 (d, J = 8.8 Hz, 1H), 7.32-7.24 (m, 1H), 6.97 (d, J = 8.0 Hz, | |
| | 1H), 6.87 (dd, J = 8.4, 18.8 Hz, 3H), 6.78-6.72 (m, 1H), 5.40-5.33 (m, | |
| | 1H), 4.37 (s, 2H), 3.64 (s, 3H), 3.13 (d, J = 9.2 Hz, 2H), 2.95-2.82 (m, | |
| | 5H), 2.76-2.53 (m, 8H), 2.46 (s, 3H), 2.30 (d, J = 3.2 Hz, 2H), 2.00 (s, | |
| | 1H), 1.85 (d, J = 11.2 Hz, 2H), 1.68 (s, 1H), 1.34 (d, J = 10.8 Hz, 2H) | |
| 350 | ¹H NMR (400 MHz, DMSO-d6) δ 11.08 (s, 1H), 9.91 (s, 1H), 8.76 (s, | A |
| | 1H), 8.55 (d, J = 8.8 Hz, 1H), 7.84 (s, 1H), 7.62 (dd, J = 4.8, 8.8 Hz, | |
| | 1H), 7.47 (d, J = 8.8 Hz, 1H), 7.28 (ddd, J = 4.4, 8.4, 10.2 Hz, 1H), 6.93 | |
| | (d, J = 8.4 Hz, 1H), 6.88-6.81 (m, 2H), 6.74 (ddd, J = 3.2, 6.4, 8.4 Hz, | |
| | 1H), 6.64 (dd, J = 2.0, 8.4 Hz, 1H), 5.29 (dd, J = 5.2, 12.8 Hz, 1H), 4.36 | |
| | (s, 2H), 3.60 (d, J = 11.6 Hz, 2H), 3.42-3.35 (m, 3H), 3.31 (s, 3H), | |
| | 2.93-2.84 (m, 5H), 2.75-2.58 (m, 7H), 2.46 (s, 3H), 2.04-1.93 (m, 1H), | |
| | 1.83 (d, J = 11.6 Hz, 2H), 1.79-1.67 (m, 1H), 1.30 (d, J = 10.4 Hz, 2H) | |
| 351 | ¹H NMR (400 MHz, DMSO-d6) δ 11.05 (s, 1H), 9.85 (s, 1H), 8.77 (s, | A |
| | 1H), 8.56 (d, J = 8.4 Hz, 1H), 8.14 (d, J = 7.2 Hz, 1H), 8.00 (d, J = 3.2 | |
| | Hz, 1H), 7.79 (s, 1H), 7.67 (d, J = 8.4 Hz, 1H), 7.45 (dd, J = 3.2, 8.8 Hz, | |
| | 1H), 6.99-6.87 (m, 3H), 6.83 (t, J = 6.8 Hz, 1H), 6.64 (dd, J = 1.6, 8.8 | |
| | Hz, 1H), 5.26 (dd, J = 5.2, 13.2 Hz, 1H), 4.59 (m, 1H), 4.37 (s, 2H), | |
| | 3.57 (d, J = 12.0 Hz, 2H), 3.13 (s, 4H), 2.92-2.83 (m, 1H), 2.69-2.56 | |
| | (m, 8H), 2.33 (d, J = 2.0 Hz, 3H), 2.25 (d, J = 6.8 Hz, 2H), 2.03-1.94 | |
| | (m, 1H), 1.88-1.79 (m, 2H), 1.73-1.64 (m, 1H), 1.45 (d, J = 6.8 Hz, | |
| | 6H), 1.34-1.22 (m, 2H) | |
| 352 | ¹H NMR (400 MHz, DMSO-d6) δ 11.08 (s, 1H), 10.26 (s, 1H), 8.91 (s, | A |
| | 1H), 8.67 (d, J = 8.4 Hz, 1H), 8.57 (s, 1H), 8.46 (dd, J = 6.0, 7.2 Hz, | |
| | 1H), 7.85 (s, 1H), 7.77 (d, J = 8.4 Hz, 1H), 7.54 (dd, J = 2.4, 10.0 Hz, | |
| | 1H), 7.30 (s, 1H), 6.99 (dt, J = 2.8, 7.6 Hz, 1H), 6.93 (d, J = 8.8 Hz, | |
| | 1H), 6.90 (d, J = 2.0 Hz, 1H), 6.64 (dd, J = 2.0 8.8 Hz, 1H), 5.31-5.23 | |
| | (m, 1H), 4.64-4.55 (m, 1H), 4.42 (s, 2H), 3.57 (d, J = 11.6 Hz, 2H), | |
| | 3.36 (s, 4H), 2.97 (s, 4H), 2.89 (s, 1H), 2.69-2.58 (m, 4H), 2.25 (d, J = | |
| | 7.2 Hz, 2H), 1.99 (d, J = 5.6 Hz, 1H), 1.84 (d, J = 11.2 Hz, 2H), 1.73- | |
| | 1.61 (m, 1H), 1.45 (d, J = 6.8 Hz, 6H), 1.29 (d, J = 11.2 Hz, 2H) | |
| 353 | ¹H NMR (400 MHz, DMSO-d6) δ 11.16-11.00 (m, 1H), 9.96 (s, 1H), | A |
| | 8.80 (s, 1H), 8.68 (d, J = 8.4 Hz, 1H), 8.44 (dd, J = 6.0, 7.2 Hz, 1H), | |
| | 7.80 (s, 1H), 7.72 (d, J = 8.8 Hz, 1H), 7.52 (dd, J = 2.8, 10.0 Hz, 1H), | |
| | 7.48 (d, J = 8.8 Hz, 1H), 6.96 (dt, J = 2.8, 7.6 Hz, 1H), 6.84 (d, J = 8.4 | |
| | Hz, 1H), 6.72 (s, 1H), 6.56-6.48 (m, 1H), 5.76 (s, 1H), 5.40 (dd, J = | |
| | 5.2, 12.8 Hz, 1H), 4.72-4.48 (m, 1H), 4.40 (s, 2H), 3.64 (d, J = 12.0 | |
| | Hz, 2H), 3.06-2.92 (m, 1H), 2.84 (s, 4H), 2.72-2.52 (m, 6H), 2.44 (s, | |
| | 3H), 2.32-2.16 (m, 3H), 2.12-2.00 (m, 1H), 1.80 (d, J = 12.0 Hz, 2H), | |
| | 1.68 (s, 1H), 1.52-1.40 (m, 6H), 1.32-1.20 (m, 2H) | |
| 354 | ¹H NMR (400 MHz, DMSO-d6) δ 11.17 (s, 1H), 9.94 (s, 1H), 8.79 (s, | A |
| | 1H), 8.66 (d, J = 8.4 Hz, 1H), 8.48-8.40 (m, 1H), 8.30-7.94 (m, 2H), | |
| | 7.82 (s, 1H), 7.71 (d, J = 8.4 Hz, 1H), 7.52 (dd, J = 2.4, 10.0 Hz, 1H), | |
| | 7.47 (d, J = 8.8 Hz, 1H), 7.21-7.14 (m, 1H), 7.07 (d, J = 8.0 Hz, 1H), | |
| | 7.01 (d, J = 8.0 Hz, 1H), 6.96 (dt, J = 2.8, 7.2 Hz, 1H), 6.84 (d, J = 8.4 | |
| | Hz, 1H), 5.41 (dd, J = 4.8, 12.4 Hz, 1H), 4.38 (s, 2H), 3.06 (d, J = 9.6 | |
| | Hz, 2H), 2.86 (s, 4H), 2.77-2.53 (m, 9H), 2.47 (s, 3H), 2.33-2.26 (m, | |
| | 2H), 2.12-2.04 (m, 1H), 1.85 (d, J = 10.4 Hz, 2H), 1.75-1.61 (m, 1H), | |
| | 1.41-1.25 (m, 2H) | |
| 355 | ¹H NMR (400 MHz, DMSO-d6) δ 11.15 ( s, 1H), 9.94 (s, 1H), 8.79 (s, | A |
| | 1H), 8.65 (d, J = 8.8 Hz, 1H), 8.44 (dd, J = 6.0, 7.2 Hz, 1H), 7.81 (s, | |
| | 1H), 7.70 (d, J = 8.4 Hz, 1H), 7.69-7.49 (m, 2H), 7.47 (d, J = 8.4 Hz, | |
| | 1H), 7.11 (d, J = 8.8 Hz, 1H), 6.96 (dt, J = 2.8, 7.6 Hz, 1H), 6.92 (s, | |
| | 1H), 6.84 ( d, J = 8.4 Hz, 2H), 5.36 (dd, J = 5.6, 12.8 Hz, 1H), 4.38 (s, | |
| | 2H), 3.57 ( d, J = 11.2 Hz, 2H), 2.98-2.88 (m, 1H), 2.85 ( s, 4H), 2.72- | |
| | 2.58 (m, 5H), 2.46 (s, 3H), 2.25 ( d, J = 7.2 Hz, 2H), 2.11-2.04 (m, | |
| | 1H), 1.83 ( d, J = 11.6 Hz, 2H), 1.72-1.64 (m, 1H), 1.32-1.23 (m, 3H), | |
| | 1.22-1.09 (m, 2H) | |
| 356 | ¹H NMR (400 MHz, DMSO-d6) δ 11.11 (s, 1H), 9.95 (s, 1H), 8.80 (s, | A |
| | 1H), 8.66 (d, J = 8.8 Hz, 1H), 8.44 (dd, J = 6.0, 7.2 Hz, 1H), 7.82 (s, | |
| | 1H), 7.71 (d, J = 8.8 Hz, 1H), 7.57-7.43 (m, 2H), 7.08-6.92 (m, 3H), | |
| | 6.85 (d, J = 8.8 Hz, 1H), 6.70 (dd, J = 1.6, 8.8 Hz, 1H), 5.33 (dd, J = 5.2, | |
| | 12.8 Hz, 1H), 4.87-4.72 (m, 2H), 4.38 (s, 2H), 3.60 (d, J = 11.2 Hz, | |
| | 2H), 3.34 (s, 2H), 2.90 (s, 5H), 2.78-2.59 (m, 7H), 2.46 (s, 3H), 2.41 | |
| | (s, 1H), 2.07-2.00 (m, 1H), 1.93-1.64 (m, 3H), 1.36-1.23 (m, 2H) | |
| 357 | ¹H NMR (400 MHz, DMSO-d6) δ 10.53 (s, 1H), 9.84 (s, 1H), 9.39 (s, | A |
| | 1H), 8.77 (s, 1H), 8.56 (d, J = 8.4 Hz, 1H), 8.52 (s, 1H), 8.41 (dd, J = | |
| | 5.6, 7.2 Hz, 1H), 8.00 (d, J = 2.8 Hz, 1H), 7.80 (s, 1H), 7.74-7.66 (m, | |
| | 2H), 7.59 (d, J = 8.4 Hz, 1H), 7.52 (dd, J = 2.4, 10.0 Hz, 1H), 7.45 (dd, | |
| | J = 2.8, 8.8 Hz, 1H), 7.25 (d, J = 7.2 Hz, 1H), 7.00-6.94 (m, 2H), 4.37 (s, | |
| | 2H), 3.90 (m, 1H), 3.70 (m, 1H), 3.14 (s, 4H), 3.01-2.71 (m, 5H), 2.57 | |
| | s, 5H), 2.34 (d, J = 6.8 Hz, 2H), 1.99-1.89 (m, 2H), 1.86-1.73 (m, | |
| | 1H), 1.61-1.45 (m, 2H) | |
| 358 | ¹H NMR (400 MHz, DMSO-d6) δ 10.99 (s, 1H), 10.06-9.84 (m, 1H), | A |
| | 9.40 (s, 1H), 8.82 (s, 1H), 8.68 (d, J = 8.4 Hz, 1H), 8.49-8.42 (m, 1H), | |
| | 8.39 (s, 1H), 7.83 (s, 1H), 7.75-7.59 (m, 3H), 7.57-7.45 (m, 2H), 7.25 | |
| | (d, J = 7.2 Hz, 1H), 7.02-6.80 (m, 2H), 4.65-4.54 (m, 1H), 4.40 (s, | |
| | 2H), 3.74-3.56 (m, 1H), 3.50-3.36 (m, 3H), 3.31-3.08 (m, 4H), 3.02- | |
| | 2.76 (m, 6H), 2.66-2.55 (m, 3H), 2.50-2.47 (m, 3H), 2.43-2.36 (m, | |
| | 1H), 2.15-2.09 (m, 1H), 2.05-1.92 (m, 2H), 1.69-1.47 (m, 2H) | |
| 359 | ¹H NMR (400 MHz, DMSO-d6) δ 11.08 (s, 1H), 10.00 (s, 1H), 8.84 (s, | A |
| | 1H), 8.68 (d, J = 8.8 Hz, 1H), 8.54 (dd, J = 0.8, 7.2 Hz, 1H), 8.44 (dd, J = | |
| | 0.8, 1.6 Hz, 1H), 8.12 (s, 1H), 7.76 (d, J = 8.4 Hz, 1H), 7.48 (d, J = | |
| | 8.8 Hz, 1H), 7.20 (dd, J = 2.0, 7.2 Hz, 1H), 6.92 (d, J = 8.8 Hz, 1H), | |
| | 6.88 (d, J = 8.4 Hz, 1H), 6.84 (d, J = 2.0 Hz, 1H), 6.60 (dd, J = 2.0, 8.8 | |
| | Hz, 1H), 5.28 (dd, J = 5.2, 12.8 Hz, 1H), 4.40 (s, 2H), 3.92 (d, J = 10.4 | |
| | Hz, 1H), 3.84 (d, J = 7.2 Hz, 1H), 3.72 (t, J = 10.0 Hz, 1H), 3.32 (s, 3H), | |
| | 3.06 (s, 4H), 3.02 (d, J = 10.8 Hz, 1H), 2.96-2.84 (m, 2H), 2.80-2.72 | |
| | (m, 1H), 2.72-2.60 (m, 4H), 2.60-2.52 (m, 8H), 2.00-1.96 (m, 1H) | |
| 360 | ¹H NMR (400 MHz, DMSO-d6) δ 11.05 (s, 1H), 9.93 (s, 1H), 8.78 (s, | A |
| | 1H), 8.65 (d, J = 8.8 Hz, 1H), 8.44 (dd, J = 6.0, 7.2 Hz, 1H), 7.81 (s, | |
| | 1H), 7.70 (d, J = 8.4 Hz, 1H), 7.55-7.42 (m, 2H), 7.01-6.89 (m, 2H), | |
| | 6.89-6.80 (m, 2H), 6.63 (dd, J = 2.0, 8.4 Hz, 1H), 5.28 (dd, J = 5.2, | |
| | 12.8 Hz, 1H), 4.38 (s, 2H), 3.91-3.77 (m, 2H), 3.59 (d, J = 11.6 Hz, | |
| | 3H), 2.93-2.83 (m, 5H), 2.73-2.53 (m, 10H), 2.30-2.22 (m, 2H), 2.05- | |
| | 1.93 (m, 1H), 1.83 (d, J = 11.2 Hz, 2H), 1.74-1.61 (m, 1H), 1.35- | |
| | 1.24 (m, 2H), 1.20 (t, J = 7.2 Hz, 3H) | |
| 361 | ¹H NMR (400 MHz, DMSO-d6) δ 11.08 (s, 1H), 9.93 (s, 1H), 8.78 (s, | A |
| | 1H), 8.65 (d, J = 8.4 Hz, 1H), 8.48-8.40 (m, 1H), 7.81 (s, 1H), 7.70 (d, | |
| | J = 8.4 Hz, 1H), 7.54-7.43 (m, 2H), 7.02-6.93 (m, 3H), 6.89 (d, J = | |
| | 6.0 Hz, 1H), 6.84 (d, J = 8.8 Hz, 1H), 5.34 (dd, J = 5.2, 12.4 Hz, 1H), | |
| | 4.38 (s, 2H), 4.19-4.06 (m, 2H), 3.06 (d, J = 10.4 Hz, 2H), 2.85 (s, 5H), | |
| | 2.80-2.61 (m, 5H), 2.58-2.53 (m, 3H), 2.46 (s, 3H), 2.28 (d, J = 6.8 | |
| | Hz, 2H), 2.05-1.96 (m, 1H), 1.91-1.82 (m, 2H), 1.74-1.63 (m, 1H), | |
| | 1.30 (d, J = 10.8 Hz, 2H), 1.21 (t, J = 7.2 Hz, 3H) | |
| 362 | ¹H NMR (400 MHz, DMSO-d6) δ 11.05 (s, 1H), 10.02 (s, 1H), 8.88 (s, | A |
| | 1H), 8.64 (d, J = 8.8 Hz, 1H), 8.48-8.36 (m, 2H), 8.09 (s, 1H), 7.65 (d, | |
| | J = 8.8 Hz, 1H), 7.50 (d, J = 8.8 Hz, 1H), 7.20 (dd, J1 = 1.6 Hz, J2 = 7.2 | |
| | Hz, 1H), 6.99-6.81 (m, 3H), 6.65 (d, J = 8.0 Hz, 1H), 5.26 (dd, J1 = 5.2 | |
| | Hz, J2 = 12.4 Hz, 1H), 4.82 (q, J = 6.4 Hz, 1H), 4.59 (quin, J = 7.2 Hz, | |
| | 1H), 3.59 (d, J = 10.4 Hz, 2H), 3.24-2.78 (m, 8H), 2.75-2.54 (m, 6H), | |
| | 2.47 (s, 3H), 2.33-2.14 (m, 1H), 2.02-1.94 (m, 1H), 1.85 (d, J = 11.6 | |
| | Hz, 3H), 1.46-1.44 (m, 6H), 1.40-1.25 (m, 2H), 0.76 (d, J = 6.8 Hz, | |
| | 3H | |
| 363 | ¹H NMR (400 MHz, DMSO-d6) δ 11.06 (s, 1H), 9.98 (s, 1H), 8.85 (s, | A |
| | 1H), 8.62 (d, J = 8.4 Hz, 1H), 8.29 (t, J = 6.2 Hz, 1H), 8.13 (s, 1H), 7.75 | |
| | (s, 1H), 7.60 (d, J = 8.4 Hz, 1H), 7.55-7.45 (m, 2H), 7.00-6.83 (m, | |
| | 4H), 6.68-6.58 (m, 1H), 5.26 (dd, J1 = 5.2 Hz, J2 = 12.8 Hz, 1H), 4.78 | |
| | (d, J = 6.8 Hz, 1H), 4.65-4.51 (m, 1H), 3.58 (d, J = 10.0 Hz, 2H), 3.06- | |
| | 2.79 (m, 6H), 2.76-2.54 (m, 8H), 2.46 (s, 3H), 2.31-2.15 (m, 1H), | |
| | 2.03-1.94 (m, 1H), 1.89-1.65 (m, 3H), 1.45 (d, J = 6.8 Hz, 6H), 1.38- | |
| | 1.25 (m, 2H), 0.79 (d, J = 6.8 Hz, 3H) | |
| 364 | ¹H NMR (400 MHz, DMSO-d6) δ 11.05 (s, 1H), 10.29 (s, 1H), 8.91 (s, | A |
| | 1H), 8.75 (d, J = 8.8 Hz, 1H), 8.56 (d, J = 7.2 Hz, 1H), 8.44 (s, 1H), 8.16 | |
| | (s, 1H), 7.80 (d, J = 8.4 Hz, 1H), 7.21 (dd, J = 1.6, 7.2 Hz, 1H), 7.10 (s, | |
| | 1H), 6.92 (d, J = 8.8 Hz, 1H), 6.88 (d, J = 2.0 Hz, 1H), 6.63 (dd, J = 1.6, | |
| | 8.8 Hz, 1H), 5.26 (dd, J = 5.2, 12.8 Hz, 1H), 4.63-4.54 (m, 1H), 4.43 | |
| | (s, 2H), 3.56 (d, J = 12.0 Hz, 2H), 3.28-3.22 (m, 3H), 2.88 (s, 1H), 2.74 | |
| | (s, 4H), 2.68-2.61 (m, 3H), 2.58 (s, 3H), 2.28-2.14 (m, 4H), 2.03-1.94 | |
| | (m, 1H), 1.83 (d, J = 10.8 Hz, 2H), 1.66 (s, 1H), 1.45 (d, J = 6.8 | |
| | Hz, 6H), 1.28 (d, J = 10.8 Hz, 2H) | |
| 365 | ¹H NMR (400 MHz, DMSO-d6) δ 11.06 (s, 1H), 10.05 (s, 1H), 8.86 (s, | A |
| | 1H), 8.66-8.56 (m, 3H), 7.74 (d, J = 8.8 Hz, 1H), 7.64-7.58 (m, 2H), | |
| | 7.46 (d, J = 8.8 Hz, 1H), 6.95-6.80 (m, 3H), 6.63 (dd, J = 2.0 Hz, 8.8 | |
| | Hz, 1H), 5.26 (dd, J = 5.2 Hz, 12.4 Hz, 1H), 4.66-4.52 (m, 3H), 3.56 | |
| | (d, J = 11.2 Hz, 2H), 3.40-3.35 (m, 1H), 3.30 (s, 1H), 2.92-2.81 (m, | |
| | 5H), 2.72-2.60 (m, 4H), 2.59-2.55 (m, 2H), 2.47-2.45 (m, 3H), 2.26 | |
| | (d, J = 6.8 Hz, 2H), 2.02-1.93 (m, 1H), 1.83 (d, J = 12.0 Hz, 2H), 1.72- | |
| | 1.61 (m, 1H), 1.45 (d, J = 6.8 Hz, 6H), 1.34-1.22 (m, 2H) | |
| 366 | ¹H NMR (400 MHz, DMSO-d6) δ 10.61 (s, 1H), 9.94 (s, 1H), 8.80 (s, | A |
| | 1H), 8.66 (d, J = 8.4 Hz, 1H), 8.53 (d, J = 7.6 Hz, 1H), 8.44 (dd, J = 6.0, | |
| | 7.2 Hz, 1H), 7.82 (s, 1H), 7.71 (d, J = 8.4 Hz, 1H), 7.55-7.45 (m, 2H), | |
| | 7.41 (s, 1H), 7.00-6.92 (m, 2H), 6.85 (d, J = 8.4 Hz, 1H), 4.38 (s, 2H), | |
| | 3.75 (t, J = 6.8 Hz, 2H), 3.47 (d, J = 10.8 Hz, 2H), 2.86 (s, 4H), 2.83- | |
| | 2.78 (m, 2H), 2.77-2.65 (m, 3H), 2.63-2.54 (m, 3H), 2.46 (s, 3H), | |
| | 2.35-2.27 (m, 2H), 1.87 (d, J = 11.6 Hz, 2H), 1.80-1.70 (m, 1H), | |
| | 1.39-1.27 (m, 2H) | |
| 367 | ¹H NMR (400 MHz, DMSO-d6) δ 11.12-10.96 (m, 1H), 9.76 (s, 1H), | A |
| | 8.72 (s, 1H), 8.52 (d, J = 8.4 Hz, 1H), 8.40 (dd, J = 6.0, 7.2 Hz, 1H), | |
| | 7.80 (s, 1H), 7.68 (d, J = 8.4 Hz, 1H), 7.52 (dd, J = 2.8, 10.0 Hz, 1H), | |
| | 6.96-6.92 (m, 2H), 6.92 (d, J = 8.8 Hz, 1H), 6.88 (d, J = 1.6 Hz, 1H), | |
| | 6.80 (d, J = 8.4 Hz, 1H), 6.60 (dd, J = 2.0, 8.8 Hz, 1H), 5.28 (dd, J = 5.2, | |
| | 12.8 Hz, 1H), 4.64-4.52 (m, 1H), 4.36 (s, 2H), 4.00-3.92 (m, 2H), | |
| | 3.60-3.52 (m, 4H), 3.24-3.16 (m, 2H), 2.92-2.80 (m, 1H), 2.72-2.52 | |
| | (m, 5H), 2.44-2.28 (m, 9H), 2.16 (d, J = 7.2 Hz, 2H), 2.04-1.92 (m, | |
| | 1H), 1.76 (d, J = 10.8 Hz, 2H), 1.68-1.56 (m, 1H), 1.44 (d, J = 6.8 Hz, | |
| | 6H), 1.32-1.16 (m, 2H) | |
| 368 | ¹H NMR (400 MHz, DMSO-d6) δ 10.37 (s, 1H), 9.93 (s, 1H), 8.78 (s, | A |
| | 1H), 8.65 (d, J = 8.4 Hz, 1H), 8.44 (t, J = 6.8 Hz, 1H), 7.81 (s, 1H), 7.70 | |
| | (d, J = 8.4 Hz, 1H), 7.56-7.49 (m, 4H), 7.49-7.44 (m, 2H), 7.44-7.38 | |
| | (m, 1H), 7.22 (d, J = 7.2 Hz, 1H), 7.02 (d, J = 8.8 Hz, 2H), 6.98-6.94 | |
| | (m, 1H), 6.84 (d, J = 8.4 Hz, 1H), 4.38 (s, 2H), 3.85 (t, J = 6.4 Hz, 2H), | |
| | 3.77 (d, J = 12.0 Hz, 2H), 2.85 (s, 4H), 2.77-2.69 (m, 4H), 2.57-2.51 | |
| | (m, 4H), 2.46 (s, 3H), 2.25 (d, J = 6.8 Hz, 2H), 1.83 (d, J = 12.0 Hz, | |
| | 2H), 1.76-1.68 (m, 1H), 1.32-1.15 (m, 2H) | |
| 369 | ¹H NMR (400 MHz, DMSO-d6) δ 11.06 (s, 1H), 9.93 (s, 1H), 8.79 (s, | A |
| | 1H), 8.65 (d, J = 8.4 Hz, 1H), 8.44 (dd, J = 6.0, 7.2 Hz, 1H), 7.81 (s, | |
| | 1H), 7.70 (d, J = 8.4 Hz, 1H), 7.52 (dd, J = 2.4, 10.0 Hz, 1H), 7.45 (d, | |
| | J = 8.4 Hz, 1H), 6.98-6.89 (m, 2H), 6.88-6.79 (m, 2H), 6.62 (dd, J = | |
| | 2.0, 8.8 Hz, 1H), 5.28 (dd, J = 5.6, 12.8 Hz, 1H), 4.38 (s, 2H), 3.60- | |
| | 3.53 (m, 2H), 3.30 (s, 6H), 2.95-2.75 (m, 8H), 2.73-2.57 (m, 3H), | |
| | 2.47-2.40 (m, 4H), 2.14 (d, J = 6.8 Hz, 2H), 2.01-1.85 (m, 5H), 1.80 | |
| | (d, J = 12.0 Hz, 2H), 1.59-1.42 (m, 3H), 1.21-1.09 (m, 2H), 0.96- | |
| | 0.83 (m, 2H) | |
| 370 | ¹H NMR (400 MHz, DMSO-d6) δ 11.04 (s, 1H), 9.96 (s, 1H), 8.82 (s, | A |
| | 1H), 8.67 (d, J = 8.4 Hz, 1H), 8.57-8.52 (m, 1H), 8.43 (s, 1H), 8.14 (s, | |
| | 1H), 7.74 (d, J = 8.4 Hz, 1H), 7.45 (d, J = 8.8 Hz, 1H), 7.19 (dd, J = 1.6, | |
| | 7.2 Hz, 1H), 6.86 (dd, J = 8.4, 15.6 Hz, 2H), 6.33 (d, J = 2.0 Hz, 1H), | |
| | 6.07 (dd, J = 2.0, 8.4 Hz, 1H), 5.24 (dd, J = 5.2, 12.8 Hz, 1H), 4.59- | |
| | 4.50 (m, 1H), 4.40 (s, 2H), 3.80 (s, 2H), 3.68 (s, 2H), 3.45 (d, J = 4.4 | |
| | Hz, 7H), 2.82 (s, 4H), 2.67-2.57 (m, 2H), 2.45 (s, 3H), 2.41 (s, 2H), | |
| | 2.34-2.30 (m, 1H), 1.97 (s, 1H), 1.92-1.84 (m, 2H), 1.43 (d, J = 6.8 | |
| | Hz, 6H) | |
| 371 | ¹H NMR (400 MHz, DMSO-d6) δ 11.04 (s, 1H), 9.96 (s, 1H), 8.82 (s, | A |
| | 1H), 8.67 (d, J = 8.4 Hz, 1H), 8.57-8.52 (m, 1H), 8.43 (s, 1H), 8.14 (s, | |
| | 1H), 7.74 (d, J = 8.4 Hz, 1H), 7.45 (d, J = 8.8 Hz, 1H), 7.19 (dd, J = 1.6, | |
| | 7.2 Hz, 1H), 6.86 (dd, J = 8.4, 15.6 Hz, 2H), 6.33 (d, J = 2.0 Hz, 1H), | |
| | 6.07 (dd, J = 2.0, 8.4 Hz, 1H), 5.24 (dd, J = 5.2, 12.8 Hz, 1H), 4.59- | |
| | 4.50 (m, 1H), 4.40 (s, 2H), 3.80 (s, 2H), 3.68 (s, 2H), 3.45 (d, J = 4.4 | |
| | Hz, 7H), 2.82 (s, 4H), 2.67-2.57 (m, 2H), 2.45 (s, 3H), 2.41 (s, 2H), | |
| | 2.34-2.30 (m, 1H), 1.97 (s, 1H), 1.92-1.84 (m, 2H), 1.43 (d, J = 6.8 | |
| | Hz, 6H) | |
| 372 | ¹H NMR (400 MHz, DMSO-d6) δ 10.96 (s, 1H), 10.07 (s, 1H), 9.93 (s, | A |
| | 1H), 8.79 (s, 1H), 8.65 (d, J = 8.6 Hz, 1H), 8.44 (dd, J = 7.2, 6.0 Hz, | |
| | 1H), 7.88 (t, J = 8.4 Hz, 1H), 7.81 (s, 1H), 7.70 (d, J = 8.6 Hz, 1H), 7.56- | |
| | 7.41 (m, 2H), 7.23 (d, J = 11.8 Hz, 1H), 7.15 (d, J = 8.4 Hz, 1H), 6.96 | |
| | (dt, J = 7.4, 2.6 Hz, 1H), 6.83 (d, J = 8.6 Hz, 1H), 4.38 (s, 2H), 3.84 (t, | |
| | J = 7.2 Hz, 1H), 3.54 (s, 2H), 2.92-2.77 (m, 4H), 2.59-2.52 (m, 6H), | |
| | 2.45 (s, 3H), 2.21-2.10 (m, 2H) | |
| 373 | ¹H NMR (400 MHz, DMSO-d6) δ 11.00 (s, 1H), 10.00-9.92 (m, 1H), | A |
| | 8.80 (s, 1H), 8.68-8.64 (m, 1H), 8.48-8.40 (m, 1H), 7.80 (s, 1H), 7.72 | |
| | (d, J = 8.4 Hz, 2H), 7.52 (dd, J = 2.8, 10.0 Hz, 1H), 7.48 (d, J = 6.8 Hz, | |
| | 1H), 7.36-7.28 (m, 1H), 6.96 (dt, J = 2.8, 7.6 Hz, 1H), 6.88-6.80 (m, | |
| | 1H), 5.12 (dd, J = 4.8, 12.8 Hz, 1H), 4.48-4.40 (m, 1H), 4.36 (s, 2H), | |
| | 4.32-4.24 (m, 1H), 4.00-3.72 (m, 3H), 3.52-3.40 (m, 1H), 3.32-3.24 | |
| | (m, 2H), 2.96-2.76 (m, 6H), 2.72-2.52 (m, 8H), 2.48 (s, 3H), 2.04- | |
| | 1.96 (m, 1H) | |
| 374 | ¹H NMR (400 MHz, DMSO-d6) δ 10.96 (s, 1H), 9.94 (s, 1H), 8.80 (s, | A |
| | 1H), 8.66 (d, J = 8.4 Hz, 1H), 8.47-8.41 (m, 1H), 7.82 (s, 1H), 7.70 (d, | |
| | J = 8.4 Hz, 1H), 7.54-7.45 (m, 2H), 7.37 (d, J = 8.4 Hz, 1H), 6.96 (dt, | |
| | J = 2.8, 7.5 Hz, 1H), 6.88-6.77 (m, 2H), 5.05 (dd, J = 5.2, 13.3 Hz, 1H), | |
| | 4.47-4.35 (m, 3H), 4.27 (d, J = 16.4 Hz, 1H), 3.67-3.46 (m, 4H), 2.99- | |
| | 2.79 (m, 7H), 2.64-2.54 (m, 6H), 2.46 (s, 3H), 2.17-2.02 (m, 2H), | |
| | 2.01-1.89 (m, 1H), 1.77-1.67 (m, 1H) | |
| 375 | ¹H NMR (400 MHz, DMSO-d6) δ 10.61 (s, 1H), 10.03-9.95 (m, 1H), | A |
| | 8.81 (s, 1H), 8.68 (d, J = 8.8 Hz, 1H), 8.44 (dd, J = 6.4, 7.4 Hz, 1H), | |
| | 7.82 (s, 1H), 7.72 (d, J = 8.4 Hz, 1H), 7.68-7.59 (m, 1H), 7.57-7.43 | |
| | (m, 2H), 7.42-7.34 (m, 1H), 6.97 (dt, J = 2.4, 7.5 Hz, 1H), 6.92-6.85 | |
| | (m, 1H), 6.40-6.31 (m, 1H), 4.49-4.11 (m, 5H), 3.93-3.54 (m, 6H), | |
| | 3.04 (br s, 4H), 2.89-2.61 (m, 7H), 2.24 (d, J = 0.8 Hz, 3H), 1.80 (br d, | |
| | J = 12.0 Hz, 4H), 1.49-1.29 (m, 2H) | |
| 376 | ¹H NMR (400 MHz, DMSO-d6) δ 10.56 (s, 1H), 9.98 (s, 1H), 8.85- | A |
| | 8.79 (m, 1H), 8.77 (d, J = 2.4 Hz, 1H), 8.72-8.64 (m, 1H), 8.50-8.40 | |
| | (m, 1H), 8.03 (d, J = 2.4 Hz, 1H), 7.82 (s, 1H), 7.75-7.68 (m, 1H), 7.55 | |
| | -7.49 (m, 2H), 7.22 (s, 1H), 7.00-6.90 (m, 2H), 6.90-6.86 (m, 1H), | |
| | 4.39 (s, 2H), 3.93 (t, J = 6.4 Hz, 2H), 3.87 (br d, J = 11.2 Hz, 2H), 3.72 | |
| | (br d, J = 1.6 Hz, 2H), 3.64-3.49 (m, 2H), 3.29-3.19 (m, 3H), 2.98 (br | |
| | s, 2H), 2.85-2.73 (m, 5H), 2.73-2.63 (m, 3H), 2.62-2.53 (m, 2H), | |
| | 2.43 (s, 3H), 1.80 (br d, J = 10.8 Hz, 2H), 1.61-1.51 (m, 1H), 1.50- | |
| | 1.38 (m, 2H) | |
| 377 | ¹H NMR (400 MHz, DMSO-d6) δ 10.95 (s, 1H), 10.00 (s, 1H), 8.82 (s, | A |
| | 1H), 8.67 (d, J = 8.4 Hz, 1H), 8.44 (dd, J = 6.0, 7.2 Hz, 1H), 7.85-7.75 | |
| | (m, 2H), 7.74-7.69 (m, 1H), 7.56-7.48 (m, 2H), 7.03-6.93 (m, 2H), | |
| | 6.89 (d, J = 8.8 Hz, 1H), 5.11-5.04 (m, 1H), 4.66-4.43 (m, 1H), 4.39 | |
| | (s, 2H), 4.27 (d, J = 17.2 Hz, 2H), 4.17-4.06 (m, 2H), 3.99 (t, J = 6.0 | |
| | Hz, 1H), 3.85-3.68 (m, 2H), 3.17-2.98 (m, 4H), 2.98-2.74 (m, 5H), | |
| | 2.69-2.52 (m, 3H), 2.48-2.28 (m, 4H), 2.02-1.92 (m, 2H), 1.35-1.21 | |
| | (m, 2H) | |
| 378 | ¹H NMR (400 MHz, DMSO-d6) δ 10.98 (s, 1H), 9.98 (s, 1H), 8.80 (s, | A |
| | 1H), 8.68 (d, J = 8.4 Hz, 1H), 8.45 (dd, J = 6.0, 7.2 Hz, 1H), 8.17 (s, | |
| | 1H), 7.82 (s, 1H), 7.72 (d, J = 8.8 Hz, 1H), 7.63 (d, J = 8.0 Hz, 1H), 7.55- | |
| | 7.49 (m, 2H), 7.27 (d, J = 8.4 Hz, 1H), 6.96 (dt, J = 2.4, 7.6 Hz, 1H), | |
| | 6.87 (d, J = 8.8 Hz, 1H), 5.09 (dd, J = 4.8, 13.2 Hz, 1H), 4.44-4.36 (m, | |
| | 3H), 4.24 (d, J = 17.6 Hz, 1H), 3.71 (s, 2H), 3.43 (d, J = 6.8 Hz, 4H), | |
| | 2.98-2.77 (m, 10H), 2.68-2.54 (m, 3H), 2.47-2.38 (m, 1H), 2.34- | |
| | 2.27 (m, 1H), 2.02-1.94 (m, 1H), 1.88-1.76 (m, 2H), 1.46-1.36 (m, | |
| | 2H) | |
| 379 | ¹H NMR (400 MHz, DMSO-d6) δ 11.03-10.88 (m, 1H), 10.02-9.92 | A |
| | (m, 1H), 8.81 (s, 1H), 8.73 (d, J = 8.8 Hz, 1H), 8.48-8.40 (m, 1H), 8.30 | |
| | (s, 1H), 7.82 (s, 1H), 7.72 (d, J = 8.4 Hz, 1H), 7.57 (d, J = 8.4 Hz, 1H), | |
| | 7.54-7.49 (m, 1H), 7.37 (d, J = 8.4 Hz, 1H), 6.96 (dt, J = 2.8, 7.6 Hz, | |
| | 1H), 6.85-6.77 (m, 2H), 5.04 (dd, J = 5.2, 13.2 Hz, 1H), 4.48-4.35 (m, | |
| | 3H), 4.31-4.23 (m, 1H), 3.10-2.98 (m, 4H), 2.86 ( s, 2H), 2.60 ( d, J = | |
| | 3.6 Hz, 1H), 2.56 ( d, J = 6.8 Hz, 2H), 2.51 ( s, 3H), 2.39 ( d, J = 7.6 Hz, | |
| | 3H), 2.12-1.91 (m, 5H), 1.74-1.59 (m, 5H) | |
| 380 | ¹H NMR (400 MHz, DMSO-d6) δ 10.96 (s, 1H), 9.99 (s, 1H), 8.81 (s, | A |
| | 1H), 8.73 (d, J = 8.4 Hz, 1H), 8.44 (dd, J = 6.0, 7.2 Hz, 1H), 7.82 (s, | |
| | 1H), 7.72 (d, J = 8.4 Hz, 1H), 7.61-7.47 (m, 2H), 7.39 (d, J = 8.0 Hz, | |
| | 1H), 6.97 (dt, J = 2.4, 7.6 Hz, 1H), 6.84 (s, 1H), 6.61 (t, J = 8.0 Hz, 1H), | |
| | 5.04 (dd, J = 4.8, 13.2 Hz, 1H), 4.48-4.34 (m, 3H), 4.27 (d, J = 16.8 | |
| | Hz, 1H), 4.17-4.01 (m, 4H), 3.02 (s, 2H), 2.96-2.81 (m, 2H), 2.58 (dd, | |
| | J = 3.6, 19.2 Hz, 6H), 2.54 (s, 3H), 2.40 (dd, J = 4.4, 13.6 Hz, 2H), 2.27 | |
| | (s, 2H), 2.01-1.90 (m, 2H), 1.75-1.58 (m, 3H) | |
| 381 | ¹H NMR (400 MHz, DMSO-d6) δ 11.06 (s, 1H), 9.93 (s, 1H), 8.79 (s, | A |
| | 1H), 8.66 (d, J = 8.4 Hz, 1H), 8.44 (dd, J = 6.0, 7.2 Hz, 1H), 7.81 (s, | |
| | 1H), 7.70 (d, J = 8.8 Hz, 1H), 7.64 (d, J = 8.8 Hz, 1H), 7.52 (dd, J = 2.4, | |
| | 10.0 Hz, 1H), 7.46 (d, J = 8.8 Hz, 1H), 6.99-6.94 (m, 1H), 6.93-6.89 | |
| | (m, 1H), 6.87-6.78 (m, 2H), 5.05 (d, J = 4.0, 12.8 Hz, 1H), 4.38 (s, | |
| | 2H), 3.69-3.58 (m, 1H), 3.56-3.47 (m, 1H), 3.43-3.37 (m, 2H), 3.11- | |
| | 3.02 (m, 1H), 2.93-2.79 (m, 5H), 2.63-2.53 (m, 5H), 2.47-2.42 (m, | |
| | 5H), 2.37-2.31 (m, 1H), 2.26-2.15 (m, 1H), 2.07-1.95 (m, 1H), | |
| | 1.76-1.61 (m, 3H) | |
| 382 | ¹H NMR (400 MHz, DMSO-d6) δ 10.80 (s, 1H), 9.92 (s, 1H), 8.80- | A |
| | 8.76 (m, 1H), 8.68 (d, J = 8.4 Hz, 1H), 8.44 (dd, J = 6.0, 7.6 Hz, 1H), | |
| | 7.96 (d, J = 2.4 Hz, 1H), 7.80 (s, 1H), 7.72 (d, J = 8.4 Hz, 1H), 7.52 (dd, | |
| | J = 2.4, 10.0 Hz, 1H), 7.48-7.44 (m, 1H), 7.40 (dd, J = 2.4, 8.8 Hz, | |
| | 1H), 6.96 (dt, J = 2.4, 7.6 Hz, 1H), 6.80 (dd, J = 8.8, 13.6 Hz, 2H), 4.36 | |
| | (s, 2H), 3.76-3.72 (m, 1H), 3.48 (s, 4H), 2.84 (d, J = 4.4 Hz, 4H), | |
| | 2.72-2.60 (m, 6H), 2.56-2.52 (m, 8H), 2.48 (s, 3H), 2.24-2.12 (m, 1H), | |
| | 2.00-1.92 (m, 1H) | |
| 383 | ¹H NMR (400 MHz, DMSO-d6) δ 11.07 (s, 1H), 10.03 (s, 1H), 8.82 (s, | A |
| | 1H), 8.71 (d, J = 7.6 Hz, 1H), 8.43 (dd, J = 6.4, 7.2 Hz, 1H), 8.14 (s, | |
| | 1H), 7.82 (s, 1H), 7.72 (d, J = 8.8 Hz, 1H), 7.67-7.60 (m, 2H), 7.52 | |
| | (dd, J = 2.4, 10.1 Hz, 1H), 7.33 (d, J = 2.0 Hz, 1H), 7.25 (dd, J = 2.0, 8.8 | |
| | Hz, 1H), 7.00-6.93 (m, 2H), 5.06 (dd, J = 5.6, 12.8 Hz, 1H), 4.38 (s, | |
| | 2H), 3.63 (d, J =2.8 Hz, 1H), 3.47 (d, J = 5.6 Hz, 2H), 3.37 (s, 4H), | |
| | 3.17-3.09 (m, 2H), 3.03-2.83 (m, 3H), 2.63-2.53 (m, 5H), 2.28 (s, 6H), | |
| | 2.05-1.99 (m, 3H), 1.75 (s, 3H), 1.68 (s, 3H), 1.58-1.51 (m, 4H) | |
| 384 | ¹H NMR (400 MHz, DMSO-d6) δ 11.08 (s, 1H), 9.96 (s, 1H), 8.80 (s, | A |
| | 1H), 8.68 (d, J = 8.8 Hz, 1H), 8.56 (d, J = 7.2 Hz, 1H), 8.44 (s, 1H), 8.16 | |
| | (s, 1H), 7.76 (d, J = 8.4 Hz, 1H), 7.48 (d, J = 8.8 Hz, 1H), 7.20 (dd, J = | |
| | 1.6, 7.2 Hz, 1H), 7.02-6.96 (m, 1H), 6.92-6.84 (m, 3H), 5.36 (dd, J = | |
| | 5.2, 12.8 Hz, 1H), 4.40 (s, 2H), 3.64 (s, 3H), 3.12 (d, J = 8.8 Hz, 2H), | |
| | 2.96-2.80 (m, 5H), 2.76-2.52 (m, 8H), 2.48 (s, 3H), 2.36-2.24 (m, | |
| | 2H), 2.04-1.96 (m, 1H), 1.92-1.80 (m, 2H), 1.68 (d, J = 2.4 Hz, 1H), | |
| | 1.40-1.28 (m, 2H) | |
| 385 | ¹H NMR (400 MHz, DMSO-d6) δ 11.06 (s, 1H), 9.97 (s, 1H), 8.85- | A |
| | 8.79 (m, 1H), 8.68 (d, J = 8.4 Hz, 1H), 8.57-8.52 (m, 1H), 8.45-8.42 | |
| | (m, 1H), 8.14 (s, 1H), 7.75 (d, J = 8.4 Hz, 1H), 7.47 (d, J = 8.4 Hz, 1H), | |
| | 7.20 (dd, J = 1.6, 7.1 Hz, 1H), 6.91 (d, J = 8.4 Hz, 1H), 6.87-6.82 (m, | |
| | 2H), 6.65 (dd, J = 2.0, 8.6 Hz, 1H), 5.25 (dd, J = 5.2, 12.6 Hz, 1H), 4.40 | |
| | (s, 2H), 3.55 (d, J = 11.2 Hz, 4H), 2.89-2.81 (m, 6H), 2.67-2.59 (m, | |
| | 4H), 2.56-2.53 (m, 2H), 2.46 (s, 3H), 2.25 (d, J = 6.8 Hz, 2H), 2.00- | |
| | 1.93 (m, 1H), 1.84 (d, J = 11.2 Hz, 2H), 1.67 (s, 1H), 1.28 (q, J = 10.8 | |
| | Hz, 2H), 1.08-1.01 (m, 2H), 0.91-0.83 (m, 2H) | |
| 386 | ¹H NMR (400 MHz, DMSO-d6) δ 11.05 (s, 1H), 9.99 (s, 1H), 8.84 (s, | A |
| | 1H), 8.63 (d, J = 8.4 Hz, 1H), 8.53 (dd, J = 0.8, 7.2 Hz, 1H), 8.44 (dd, J = | |
| | 0.8, 1.6 Hz, 1H), 8.15 (s, 1H), 8.03 (d, J = 11.2 Hz, 1H), 7.76 (d, J = | |
| | 8.4 Hz, 1H), 7.21 (dd, J = 1.6, 7.2 Hz, 1H), 7.03 (d, J = 13.6 Hz, 1H), | |
| | 6.96-6.84 (m, 2H), 6.63 (dd, J = 2.0, 8.8 Hz, 1H), 5.26 (dd, J = 5.2, | |
| | 12.8 Hz, 1H), 4.68-4.52 (m, 1H), 4.41 (s, 2H), 3.56 (d, J = 11.6 Hz, | |
| | 2H), 3.04 (s, 4H), 2.92-2.83 (m, 1H), 2.73-2.53 (m, 8H), 2.25 (d, J = | |
| | 6.8 Hz, 2H), 2.01-1.93 (m, 1H), 1.83 (d, J = 11.6 Hz, 2H), 1.71-1.59 | |
| | (m, 1H), 1.45 (d, J = 6.8 Hz, 6H), 1.36-1.21 (m, 2H) | |
| 387 | ¹H NMR (400 MHz, DMSO-d6) δ 10.94 (d, J = 2.4 Hz, 1H), 9.99 (s, | A |
| | 1H), 8.82 (s, 1H), 8.74 (d, J = 8.4 Hz, 1H), 8.48-8.40 (m, 1H), 7.82 (s, | |
| | 1H), 7.72 (d, J = 8.4 Hz, 1H), 7.57 (d, J = 8.4 Hz, 1H), 7.55-7.47 (m, | |
| | 2H), 6.97 (dt, J = 2.4, 7.2 Hz, 1H), 6.83 (d, J = 8.4 Hz, 1H), 6.66-6.61 | |
| | (m, 2H), 5.08-4.99 (m, 1H), 4.39 (s, 2H), 4.34-4.27 (m, 1H), 4.23- | |
| | 4.15 (m, 1H), 3.48 (s, 2H), 3.11-2.97 (m, 4H), 2.95-2.84 (m, 1H), | |
| | 2.71-2.59 (m, 4H), 2.71-2.59 (m, 1H), 2.44-2.29 (m, 4H), 2.17-2.04 | |
| | (m, 3H), 1.99-1.93 (m, 1H), 1.77-1.61 (m, 5H) | |
| 388 | ¹H NMR (400 MHz, DMSO-d6) δ 10.89 (d, J = 12.4 Hz, 1H), 9.97 (s, | A |
| | 1H), 8.81 (s, 1H), 8.73 (d, J = 8.4 Hz, 1H), 8.47-8.42 (m, 1H), 7.82 (s, | |
| | 1H), 7.72 (d, J = 8.8 Hz, 2H), 7.58-7.48 (m, 2H), 7.00-6.93 (m, 1H), | |
| | 6.88 (d, J = 8.8 Hz, 1H), 6.82 (d, J = 8.4 Hz, 1H), 4.70 (dd, J = 4.8, 12.4 | |
| | Hz, 1H), 4.53-4.42 (m, 1H), 4.38 (s, 2H), 3.66 (s, 4H), 3.31 (s, 10H), | |
| | 3.03 (d, J = 10.4 Hz, 3H), 2.83-2.72 (m, 3H), 2.64 (s, 1H), 2.09 (t, J = | |
| | 10.4 Hz, 2H), 1.98 (s, 1H), 1.71-1.59 (m, 4H), 1.39 (dd, J = 6.8, 14.0 | |
| | Hz, 3H) | |
| 389 | ¹H NMR (400 MHz, DMSO-d6) δ 10.82 (s, 1H), 9.98 (s, 1H), 8.82 (s, | A |
| | 1H), 8.73 (d, J = 8.4 Hz, 1H), 8.47-8.42 (m, 1H), 8.18 (s, 1H), 7.96 (d, | |
| | J = 2.0 Hz, 1H), 7.82 (s, 1H), 7.72 (d, J = 8.4 Hz, 1H), 7.57-7.50 (m, | |
| | 2H), 7.39 (dd, J = 2.0, 8.8 Hz, 1H), 6.96 (d, J = 2.4 Hz, 1H), 6.85-6.77 | |
| | (m, 2H), 4.39 (s, 2H), 3.74 (dd, J = 4.8, 12.4 Hz, 1H), 3.45 (s, 4H), 3.07 | |
| | (d, J = 10.8 Hz, 3H), 2.73-2.60 (m, 3H), 2.60-2.51 (m, 10H), 2.22- | |
| | 2.12 (m, 3H), 2.01-1.93 (m, 1H), 1.73-1.59 (m, 4H) | |
| 390 | ¹H NMR (400 MHz, DMSO-d6) δ11.04 (s, 1H), 9.64 (s, 1H), 9.00 (s, | A |
| | 1H), 8.44 (s, 1H), 8.40 (s, 1H), 8.36 (s, 1H), 8.28 (s, 1H), 8.20 (s, 1H), | |
| | 7.48 (d, J = 8.8 Hz, 1H), 6.92 (d, J = 8.8 Hz, 1H), 6.88 (d, J = 2.0 Hz, | |
| | 1H), 6.80 (d, J = 8.8 Hz, 1H), 6.64 (dd, J = 2.0, 8.8 Hz, 1H), 5.28 (dd, | |
| | J = 5.2, 12.8 Hz, 1H), 4.64-4.52 (m, 1H), 4.08-4.04 (m, 1H), 4.00 (s, | |
| | 3H), 3.96-3.92 (m, 1H), 3.56 (d, J = 12.0 Hz, 3H), 2.92-2.80 (m, 5H), | |
| | 2.72-2.56 (m, 6H), 2.48 (s, 3H), 2.28 (d, J = 6.8 Hz, 2H), 2.04-1.96 | |
| | (m, 1H), 1.84 (d, J = 12.0 Hz, 2H), 1.76-1.60 (m, 2H), 1.44 (d, J = 6.8 | |
| | Hz, 6H), 1.36-1.20 (m, 2H), 0.88-0.84 (m, 1H), 0.80-0.72 (m, 2H), | |
| | 0.68-0.60 (m, 1H) | |
| 391 | ¹H NMR (400 MHz, DMSO-d6) δ 11.05 (s, 1H), 9.80 (s, 1H), 8.61 (s, | A |
| | 1H), 8.51 (s, 1H), 7.96-7.90 (m, 1H), 7.71 (s, 1H), 7.56-7.48 (m, 2H), | |
| | 6.97-6.86 (m, 4H), 6.65 (d, J = 7.6 Hz, 1H), 5.27 (dd, J = 5.4, 12.4 Hz, | |
| | 1H), 4.63-4.55 (m, 1H), 4.27 (d, J = 18.4 Hz, 1H), 3.90 (d, J = 18.4 Hz, | |
| | 1H), 3.57 (d, J = 11.2 Hz, 2H), 3.37-3.35 (m, 2H), 3.24-3.01 (m, 8H), | |
| | 2.92-2.84 (m, 1H), 2.72-2.57 (m, 5H), 2.55-2.51 (m, 3H), 2.09 (s, | |
| | 3H), 2.02-1.94 (m, 1H), 1.80 (d, J = 11.6 Hz, 2H), 1.75-1.66 (m, 2H), | |
| | 1.45 (d, J = 6.8 Hz, 6H), 1.36 (d, J = 10.4 Hz, 2H) | |
| 392 | ¹H NMR (400 MHz, DMSO-d6) δ 11.06 (s, 1H), 10.29 (s, 1H), 8.91 (s, | A |
| | 1H), 8.68 (d, J = 8.4 Hz, 1H), 8.58-8.54 (m, 2H), 8.45 (s, 1H), 8.17 (s, | |
| | 1H), 7.81 (d, J = 8.4 Hz, 1H), 7.31 (s, 1H), 7.22 (dd, J = 1.6, 7.2 Hz, | |
| | 1H), 6.93 (d, J = 8.8 Hz, 1H), 6.89 (d, J = 2.0 Hz, 1H), 6.64 (dd, J = 2.0, | |
| | 8.8 Hz, 1H), 5.27 (dd, J = 5.2, 12.8 Hz, 1H), 4.64-4.55 (m, 1H), 4.44 | |
| | (s, 2H), 3.57 (d, J = 11.6 Hz, 2H), 2.98 (s, 4H), 2.89 (s, 1H), 2.74-2.53 | |
| | (m, 8H), 2.29 (d, J = 6.4 Hz, 2H), 1.99 (d, J = 10.4 Hz, 1H), 1.84 (d, J = | |
| | 11.2 Hz, 2H), 1.68 (s, 1H), 1.46 (d, J = 6.8 Hz, 6H), 1.29 (d, J = 10.0 | |
| | Hz, 2H) | |
| 393 | ¹H NMR (400 MHz, DMSO-d6) δ 11.06 (s, 1H), 9.95-9.84 (m, 1H), | A |
| | 8.78 ( s, 1H), 8.58 ( d, J = 8.4 Hz, 1H), 8.41 (dd, J = 6.0, 7.6 Hz, 1H), | |
| | 8.12-7.96 (m, 1H), 7.81 (s, 1H), 7.69 (d, J = 8.4 Hz, 1H), 7.58-7.40 | |
| | (m, 2H), 7.09-6.87 (m, 4H), 6.65 ( d, J = 8.8 Hz, 1H), 5.27 ( dd, J = | |
| | 5.6, 12.4 Hz, 1H), 4.59 (td, J = 6.8, 13.6 Hz, 1H), 4.38 (s, 2H), 3.84- | |
| | 3.69 (m, 1H), 3.60 ( dd, J = 0.8, 5.6 Hz, 2H), 3.22-3.09 (m, 4H), 2.92- | |
| | 2.83 (m, 1H), 2.73-2.51 (m, 8H), 2.31-2.15 (m, 1H), 2.05-1.76 (m, | |
| | 4H), 1.45 (d, J = 6.8 Hz, 6H), 1.41-1.20 (m, 2H) | |
| 394 | ¹H NMR (400 MHz, DMSO-d6) δ 11.11 (s, 1H), 9.88 (s, 1H), 8.79 (s, | A |
| | 1H), 8.58 (d, J = 8.8 Hz, 1H), 8.41 (dd, J = 6.0, 7.2 Hz, 1H), 8.03 (s, | |
| | 1H), 7.81 (s, 1H), 7.69 (d, J = 8.4 Hz, 1H), 7.52 (dd, J = 2.4, 10.0 Hz, | |
| | 1H), 7.48 (d, J = 9.2 Hz, 1H), 7.02-6.95 (m, 2H), 6.86 (d, J = 8.4 Hz, | |
| | 1H), 6.74 (t, J = 8.0 Hz, 1H), 5.34 (dd, J = 5.2, 12.8 Hz, 1H), 4.38 (s, | |
| | 2H), 3.47 (d, J = 1.6 Hz, 3H), 3.34 (s, 5H), 3.25 (d, J = 10.8 Hz, 3H), | |
| | 3.18-3.02 (m, 3H), 2.88 (dd, J = 4.4, 17.2 Hz, 2H), 2.72-2.58 (m, 5H), | |
| | 2.04-1.96 (m, 1H), 1.85 (d, J = 11.6 Hz, 2H), 1.46-1.29 (m, 2H) | |
| 395 | ¹H NMR (400 MHz, DMSO-d6) δ 11.07 (s, 1H), 9.74 (s, 1H), 8.60 (s, | A |
| | 1H), 8.49 (s, 1H), 8.15 (s, 2H), 7.97-7.91 (m, 1H), 7.70 (s, 1H), 7.53 | |
| | (dd, J = 2.4, 10.0 Hz, 1H), 7.47 (d, J = 8.8 Hz, 1H), 6.98-6.80 (m, 4H), | |
| | 6.63 ( d, J = 7.2 Hz, 1H), 5.27 ( dd, J = 5.2, 12.8 Hz, 1H), 4.66-4.51 | |
| | (m, 1H), 4.27 ( d, J = 18.0 Hz, 1H), 3.90 ( d, J = 18.0 Hz, 1H), 3.62- | |
| | 3.49 (m, 2H), 2.96-2.76 (m, 5H), 2.75-2.51 (m, 8H), 2.46 (s, 3H), | |
| | 2.35-2.23 (m, 2H), 2.09 (s, 3H), 2.03-1.92 (m, 1H), 1.83 ( d, J = 11.6 | |
| | Hz, 2H), 1.68 ( d, J = 3.2 Hz, 1H), 1.45 (d, J = 6.8 Hz, 6H), 1.33-1.22 | |
| | (m, 2H) | |
| 396 | ¹H NMR (400 MHz, DMSO-d6) δ 10.94 (d, J = 4.0 Hz, 1H), 9.93 (s, | A |
| | 1H), 8.79 (s, 1H), 8.65 (d, J = 8.4 Hz, 1H), 8.47-8.40 (m, 1H), 8.13 (s, | |
| | 1H), 7.81 (s, 1H), 7.70 (d, J = 8.8 Hz, 1H), 7.52 (J = 2.4, 10.0 Hz, 1H), | |
| | 7.45 (d, J = 8.8 Hz, 1H), 7.24-7.17 (m, 1H), 7.15-7.05 (m, 1H), 6.96 | |
| | (J = 2.4, 7.6 Hz, 1H), 6.83 (d, J = 8.4 Hz, 1H), 5.04 (dd, J = 4.4, 13.2 | |
| | Hz, 1H), 4.50-4.41 (m, 2H), 4.38 (s, 2H), 4.33-4.20 (m, 2H), 4.16- | |
| | 3.94 (m, 3H), 3.12 (s, 2H), 2.97-2.76 (m, 7H), 2.70-2.53 (m, 7H), | |
| | 2.45 (s, 3H), 2.43-2.36 (m, 1H), 2.01-1.88 (m, 1H) | |
| 397 | ¹H NMR (400 MHz, DMSO-d6) δ 1.55-1.78 (m, 2 H) 1.89-2.11 (m, 3 | B |
| | H) 2.57-2.78 (m, 6 H) 2.79-2.97 (m, 3 H) 2.97-3.23 (m, 5 H) 3.32 (s, | |
| | 3 H) 3.65-3.80 (m, 2 H) 4.39 (s, 2 H) 5.31 (br dd, J=12.82, 5.32 Hz, 1 | |
| | H) 6.62-6.80 (m, 2 H) 6.86-7.00 (m, 3 H) 7.30 (ddd, J=10.32, 8.44, | |
| | 4.25 Hz, 1 H) 7.53-7.73 (m, 2 H) 7.86 (s, 1 H) 8.34 (d, J=8.63 Hz, 1 H) | |
| | 8.83 (s, 1 H) 10.03 (br s, 1 H) 11.09 (s, 1 H) | |
| 398 | ¹H NMR (400 MHz, DMSO-d6) δ 11.08 (s, 1H), 9.97 (s, 1H), 8.83 (s, | B |
| | 1H), 8.68 (d, J = 8.4 Hz, 1H), 8.57-8.53 (m, 1H), 8.45-8.42 (m, 1H), | |
| | 8.16 (s, 1H), 8.14 (s, 1H), 7.75 (d, J = 8.4 Hz, 1H), 7.45 (d, J = 8.8 Hz, | |
| | 1H), 7.20 (dd, J = 1.6, 7.2 Hz, 1H), 6.93 (d, J = 8.4 Hz, 1H), 6.85 (dd, J = | |
| | 3.2, 5.2 Hz, 2H), 6.64 (dd, J = 2.0, 8.8 Hz, 1H), 5.29 (dd, J = 5.2, 12.8 | |
| | Hz, 1H), 4.41 (s, 2H), 3.65 (d, J = 11.6 Hz, 3H), 3.31 (s, 3H), 2.93- | |
| | 2.87 (m, 1H), 2.84 (s, 4H), 2.70-2.58 (m, 7H), 2.46 (s, 3H), 2.42-2.36 | |
| | (m, 1H), 2.03-1.95 (m, 1H), 1.90 (d, J = 11.2 Hz, 2H), 1.67-1.53 (m, | |
| | 2H) | |
| 399 | ¹H NMR (400 MHz, DMSO-d6) δ 11.06 (s, 1H), 10.26 (s, 1H), 8.89 (s, | B |
| | 1H), 8.74 (d, J = 8.4 Hz, 1H), 8.46 (dd, J = 6.0, 7.2 Hz, 1H), 8.15 (s, | |
| | 1H), 7.84 (s, 1H), 7.77 (d, J = 8.4 Hz, 1H), 7.52 (dd, J = 2.4, 10.0 Hz, | |
| | 1H), 7.09 (s, 1H), 6.97 (dt, J = 2.8, 7.6 Hz, 1H), 6.91 (d, J = 8.4 Hz, | |
| | 1H), 6.84 (d, J = 2.0 Hz, 1H), 6.66 (dd, J = 2.0, 8.8 Hz, 1H), 5.25 (dd, J = | |
| | 5.2, 12.4 Hz, 1H), 4.41 (s, 2H), 3.61 (d, J = 11.2 Hz, 2H), 3.23 (d, J = | |
| | 9.2 Hz, 3H), 2.86-2.81 (m, 3H), 2.77 (d, J = 10.0 Hz, 2H), 2.69-2.61 | |
| | (m, 4H), 2.57 (s, 3H), 2.45-2.37 (m, 3H), 2.01-1.94 (m, 1H), 1.93- | |
| | 1.86 (m, 2H), 1.66-1.56 (m, 2H), 1.08-1.01 (m, 2H), 0.91-0.84 (m, | |
| | 2H) | |
| 400 | ¹H NMR (400 MHz, DMSO-d6) δ δ: 11.06 (s, 1H), 10.26 (s, 1H), 8.89 | B |
| | (s, 1H), 8.73 (d, J = 8.4 Hz, 1H), 8.46 (dd, J = 6.0, 7.2 Hz, 1H), 7.84 (s, | |
| | 1H), 7.76 (d, J = 8.4 Hz, 1H), 7.52 (dd, J = 2.4, 10.0 Hz, 1H), 7.08 (s, | |
| | 1H), 6.97 (dt, J = 2.4, 7.4 Hz, 1H), 6.92 (d, J = 8.4 Hz, 1H), 6.88 (d, J = | |
| | 1.6 Hz, 1H), 6.62 (dd, J = 1.6, 8.7 Hz, 1H), 5.26 (dd, J = 5.2, 12.8 Hz, | |
| | 1H), 4.63-4.54 (m, 1H), 4.41 (s, 2H), 3.55 (d, J = 11.6 Hz, 4H), 3.28- | |
| | 3.23 (m, 2H), 2.91-2.83 (m, 1H), 2.73 (d, J = 8.4 Hz, 3H), 2.68-2.61 | |
| | (m, 3H), 2.58-2.55 (m, 3H), 2.27-2.13 (m, 4H), 2.02-1.93 (m, 1H), | |
| | 1.83 (d, J = 11.2 Hz, 2H), 1.65 (s, 1H), 1.45 (d, J = 6.8 Hz, 6H), 1.34- | |
| | 1.21 (m, 2H) | |
| 401 | ¹H NMR (400 MHz, DMSO-d6) δ 11.05 (s, 1H), 9.98 (s, 1H), 8.81 (s, | B |
| | 1H), 8.68 (d, J = 8.5 Hz, 1H), 8.49-8.39 (m,1H), 7.82 (s, 1H), 7.71 (d, | |
| | J = 8.5 Hz, 1H), 7.56-7.45 (m, 2H), 7.04-6.81 (m, 4H), 6.67-6.56 (m, | |
| | 1H), 5.25 (br dd,J = 5.3, 12.6 Hz, 1H), 4.57 (quin, J = 6.8 Hz, 1H), 4.39 | |
| | (s, 2H), 3.70 (br s, 2H), 3.43 (br d, J = 9.6 Hz, 4H), 3.02-2.59(m, 11H), | |
| | 2.25-2.17 (m, 1H), 2.00-1.93 (m, 1H), 1.82-1.73 (m, 2H), 1.44 (d, | |
| | J = 6.8 Hz, 6H), 1.37-1.21 (m, 3H) | |
| 402 | ¹H NMR (400 MHz, DMSO-d6) δ 11.08 (s, 1H), 9.99 (s, 1H), 8.81 (s, | B |
| | 1H), 8.68 (d, J = 8.8 Hz, 1H), 8.45 (dd, J = 6.0, 7.2 Hz, 1H), 7.82 (s, | |
| | 1H), 7.72 (d, J = 8.4 Hz, 1H), 7.60-7.45 (m, 2H), 7.04-6.78 (m, 5H), | |
| | 5.34 (dd, J = 5.6, 12.4 Hz, 1H), 4.39 (s, 2H), 3.71 (s, 2H), 3.60 (s, 3H), | |
| | 3.48-3.37 (m, 2H), 3.18-3.04 (m, 2H), 3.01-2.77 (m, 8H), 2.76-2.58 | |
| | (m, 5H), 2.23-2.11 (m, 1H), 2.05-1.93 (m, 1H), 1.91-1.75 (m, 2H), | |
| | 1.44-1.29 (m, 2H) | |
| 403 | ¹H NMR (400 MHz, DMSO-d6) δ 10.96 (s, 1H), 9.94 (s, 1H), 8.82- | B |
| | 8.73 (m, 1H), 8.70-8.63 (m, 1H), 8.50-8.38 (m, 1H), 7.81 (s, 1H), | |
| | 7.75-7.64 (m, 1H), 7.56-7.44 (m, 2H), 7.40 (d, J = 8.0 Hz, 1H), 6.96 | |
| | (dt, J = 2.4, 7.6 Hz, 1H), 6.88-6.80 (m, 1H), 6.69-6.61 (m, 1H), 5.01- | |
| | 5.01 (m, 1H), 5.05 (dd, J = 4.8, 13.2 Hz, 1H), 4.48-4.24 (m, 4H), 4.21- | |
| | 4.11 (m, 2H), 3.90 (br d, J = 2.0 Hz, 2H), 3.45-3.36 (m, 2H), 2.93- | |
| | 2.81 (m, 5H), 2.55 (br dd, J = 1.6, 5.2 Hz, 3H), 2.47 (s, 3H), 2.40 (br dd, | |
| | J = 4.0, 12.4 Hz, 1H), 2.00-1.93 (m, 1H) | |
| 404 | ¹H NMR (400 MHz, DMSO-d6) δ 11.06 (s, 1H), 9.80 (s, 1H), 8.70 (s, | B |
| | 1H), 8.46 (d, J = 8.4 Hz, 1H), 8.00 (d, J = 2.4 Hz, 1H), 7.83 (s, 1H), 7.60 | |
| | (dd, J = 4.4, 8.8 Hz, 1H), 7.44 (dd, J = 2.8, 9.2 Hz, 1H), 7.27 (m, 1H), | |
| | 7.00-6.90 (m, 2H), 6.85 (d, J = 2.0 Hz, 1H), 6.74 (m, 1H), 6.65 (dd, J = | |
| | 1.6, 8.8 Hz, 1H), 5.36-5.22 (m, 1H), 4.35 (s, 2H), 3.65 (d, J = 12.0 Hz, | |
| | 2H), 3.11 (s, 4H), 2.95-2.83 (m, 1H), 2.75-2.57 (m, 9H), 2.54 (d, J = | |
| | 3.2 Hz, 2H), 2.41-2.34 (m, 1H), 2.02-1.86 (m, 3H), 1.68-1.52 (m, | |
| | 2H) | |
| 405 | ¹H NMR (400 MHz, DMSO-d6) δ 11.00 (s, 1H), 10.04 (s, 1H), 8.84 (s, | B |
| | 1H), 8.74 (d, J = 8.4 Hz, 1H), 8.43 (t, J = 6.4 Hz, 1H), 8.20 (s, 1H), 7.82 | |
| | (s, 1H), 7.70 (d, J = 8.4 Hz, 1H), 7.61 (dd, J = 8.4, 11.6 Hz, 2H), 7.52 | |
| | (dd, J = 2.4, 9.6 Hz, 1H), 7.25 (d, J = 8.0 Hz, 1H), 7.01-6.92 (m, 2H), | |
| | 5.09 (dd, J = 4.8, 13.2 Hz, 1H), 4.45-4.33 (m, 3H), 4.24 (d, J = 17.6 | |
| | Hz, 1H), 3.74-3.59 (m, 4H), 3.47 (d, J = 6.4 Hz, 2H), 3.31 (d, J = 4.0 | |
| | Hz, 2H), 3.11 (s, 2H), 2.95 (d, J = 7.6 Hz, 4H), 2.79 (s, 2H), 2.63-2.52 | |
| | (m, 2H), 2.46-2.41 (m, 1H), 2.36 (s, 7H), 2.02-1.94 (m, 1H), 1.83 (d, | |
| | J = 9.2 Hz, 2H), 1.39 (d, J = 9.6 Hz, 2H) | |
| 406 | ¹H NMR (400 MHz, DMSO-d6) δ 11.07 (s, 1H), 9.84 (s, 1H), 8.76 (s, | C |
| | 1H), 8.55 (d, J = 8.8 Hz, 1H), 8.41 (dd, J = 6.0, 7.2 Hz, 1H), 8.17 (s, | |
| | 1H), 8.01 (d, J = 3.2 Hz, 1H), 7.81 (s, 1H), 7.68 (d, J = 8.4 Hz, 1H), | |
| | 7.55-7.42 (m, 2H), 7.02-6.91 (m, 3H), 6.84 (d, J = 2.0 Hz, 1H), 6.63 (dd, J = | |
| | 2.0, 8.8 Hz, 1H), 5.29 (dd, J = 5.2, 12.8 Hz, 1H), 4.37 (s, 2H), 3.68- | |
| | 3.65 (m, 2H), 3.31 (s, 3H), 3.10 (s, 4H), 2.90-2.85 (m, 1H), 2.73-2.57 | |
| | (m, 8H), 2.43-2.36 (m, 1H), 2.02-1.87 (m, 3H), 1.67-1.50 (m, 2H) | |
| 407 | ¹H NMR (400 MHz, DMSO-d6) δ 11.05-10.96 (m, 1H), 9.98-9.90 | C |
| | (m, 1H), 8.82-8.77 (m, 1H), 8.70-8.64 (m, 1H), 8.48-8.41 (m, 1H), | |
| | 7.85-7.80 (m, 1H), 7.74-7.69 (m, 1H), 7.65-7.58 (m, 1H), 7.56-7.43 | |
| | (m, 3H), 7.01-6.93 (m, 1H), 6.88-6.81 (m, 1H), 5.19-5.07 (m, 1H), | |
| | 4.63-4.50 (m, 1H), 4.43-4.36 (m, 3H), 4.35-4.25 (m, 1H), 3.60-3.46 | |
| | (m, 4H), 3.06-2.96 (m, 3H), 2.96-2.84 (m, 2H), 2.64 (m, 4H), 2.59 (s, | |
| | 2H), 2.47 (s, 3H), 2.44-2.38 (m, 1H), 2.32-2.18 (m, 2H), 2.08-1.98 | |
| | (m, 4H), 1.98-1.88 (m, 3H), 1.68-1.59 (m, 2H) | |
| 408 | ¹H NMR (400 MHz, DMSO-d6) δ 11.11 (s, 1H), 9.93 (s, 1H), 8.78 (s, | C |
| | 1H), 8.66 (d, J = 8.4 Hz, 1H), 8.43 (dd, J = 6.0, 7.6 Hz, 1H), 7.81 (s, | |
| | 1H), 7.78-7.73 (m, 1H), 7.70 (d, J = 8.4 Hz, 2H), 7.52 (dd, J = 2.4, 10.0 | |
| | Hz, 1H), 7.45 (d, J = 8.4 Hz, 1H), 6.99-6.63 (m, 1H), 6.83 (d, J = 8.4 | |
| | Hz, 1H), 5.15-5.07 (m, 1H), 4.38 (s, 2H), 4.25 (s, 1H), 3.60-3.43 (m, | |
| | 4H), 3.25-3.04 (m, 4H), 3.01-2.95 (m, 2H), 2.91-2.84 (m, 1H), 2.70 | |
| | (s, 3H), 2.68-2.61 (m, 2H), 2.60-2.54 (m, 2H), 2.46 (s, 3H), 2.44- | |
| | 2.37 (m, 1H), 2.29-2.13 (m, 2H), 2.07-2.01 (m, 1H), 1.99-1.85 (m, | |
| | 4H), 1.79 (d, J = 10.0 Hz, 2H), 1.69-1.55 (m, 2H) | |
| 409 | ¹H NMR (400 MHz, DMSO-d6) δ 11.11 (s, 1H), 9.97 (s, 1H), 8.82 (s, | C |
| | 1H), 8.68 (d, J = 8.4 Hz, 1H), 8.49-8.41 (m, 1H), 8.26 (s, 1H), 7.82 (s, | |
| | 1H), 7.72 (d, J = 8.4 Hz, 1H), 7.56-7.45 (m, 2H), 7.06-6.93 (m, 3H), | |
| | 6.89-6.76 (m, 2H), 5.66 (s, 1H), 5.38 (dd, J = 5.2, 12.4 Hz, 1H), 4.39 | |
| | (s, 2H), 3.95-3.90 (m, 1H), 3.88-3.83 (m, 1H), 3.76-3.68 (m, 2H), | |
| | 3.20-3.11 (m, 3H), 3.02 (d, J = 11.2 Hz, 1H), 2.88 (d, J = 11.6 Hz, 2H), | |
| | 2.82-2.62 (m, 7H), 2.60-2.53 (m, 5H), 2.40 (s, 2H), 2.03-1.97 (m, | |
| | 1H) | |
| 410 | ¹H NMR (400 MHz, DMSO-d6) δ 11.01 (s, 1 H), 9.98 (s, 1 H), 8.82 (s, | C |
| | 1 H), 8.69 (d, J = 8.4 Hz, 1 H), 8.45 (m, 1 H), 7.83 (s, 1 H), 7.72 (d, J = 8.4 | |
| | Hz, 1 H), 7.48-7.58 (m, 4 H), 6.98 (m, 1 H), 6.88 (d, J = 8.4 Hz, 1 H), | |
| | 5.11 (m, 1 H), 4.55 (d, J = 8.4 Hz, 1 H), 4.35-4.43 (m, 3 H), 3.99 (d, | |
| | J = 2.0 Hz, 2 H), 3.70-3.78 (m, 1 H), 3.09-3.23 (m, 2 H), 2.82-3.05 | |
| | (m, 5 H), 2.75-2.81 (m, 1 H), 2.59-2.75 (m, 3 H), 2.55 (s, 5 H), 2.37- | |
| | 2.47 (m, 2 H), 1.98-2.04 (m, 1 H), 1.81 (s, 3 H) | |
| 411 | ¹H NMR (400 MHz, DMSO-d6) δ 10.60 (s, 1H), 9.92 (s, 1H), 8.80 (s, | C |
| | 1H), 8.65 (d, J = 8.4 Hz, 1H), 8.44 (t, J = 6.8 Hz, 1H), 8.06 (d, J = 7.6 | |
| | Hz, 1H), 7.82 (s, 1H), 7.70 (d, J = 8.8 Hz, 1H), 7.52 (dd, J = 2.0, 9.6 Hz, | |
| | 1H), 7.44 (d, J = 8.4 Hz, 1H), 7.29 (s, 1H), 6.96 (dt, J = 2.0, 7.6 Hz, | |
| | 1H), 6.90 (d, J = 6.8 Hz, 1H), 6.84 (d, J = 8.4 Hz, 1H), 6.68 (s, 1H), 4.38 | |
| | (s, 2H), 3.75 (t, J = 6.4 Hz, 2H), 3.23 (s, 4H), 3.02 (d, J = 10.4 Hz, 2H), | |
| | 2.80 (t, J = 6.4 Hz, 2H), 2.69-2.58 (m, 3H), 2.53 (s, 3H), 2.45 (s, 3H), | |
| | 2.27 (d, J = 6.8 Hz, 2H), 1.84 (d, J = 11.6 Hz, 2H), 1.73-1.63 (m, 1H), | |
| | 1.38-1.25 (m, 2H) | |
| 412 | ¹H NMR (400 MHz, DMSO-d6) δ 11.08 (s, 1H), 9.96 (s, 1H), 8.80 (s, | C |
| | 1H), 8.68 (d, J = 8.4 Hz, 1H), 8.48-8.40 (m, 1H), 7.80 (s, 1H), 7.72 (d, | |
| | J = 8.4 Hz, 1H), 7.56-7.44 (m, 2H), 7.02-6.92 (m, 3H), 6.92-6.84 | |
| | (m, 2H), 5.40-5.32 (m, 1H), 4.40 (s, 2H), 3.92 (d, J = 11.6 Hz, 1H), | |
| | 3.88-3.76 (m, 1H), 3.72-3.68 (m, 1H), 3.64-3.60 (m, 3H), 3.06-2.80 | |
| | (m, 10H), 2.76-2.68 (m, 2H), 2.64-2.52 (m, 4H), 2.44-2.32 (m, 4H), | |
| | 2.02-1.94 (m, 1H) | |
| 413 | ¹H NMR (400 MHz, DMSO-d6) δ 11.08 (s, 1H), 9.96 (s, 1H), 8.80 (s, | C |
| | 1H), 8.68 (d, J = 8.8 Hz, 1H), 8.44 (t, J = 6.8 Hz, 1H), 8.20 (s, 1H), 7.80 | |
| | (s, 1H), 7.72 (d, J = 8.8 Hz, 1H), 7.56-7.44 (m, 2H), 7.16 (s, 1H), 7.02- | |
| | 6.92 (m, 2H), 6.92-6.84 (m, 2H), 5.36-5.24 (m, 1H), 4.60 (m, J = 7.2 | |
| | Hz, 1H), 4.36 (s, 2H), 3.92 (d, J = 11.0 Hz, 1H), 3.84-3.76 (m, 1H), | |
| | 3.72 (t, J = 10.4 Hz, 1H), 3.10-2.92 (m, 4H), 2.88 (d, J = 11.6 Hz, 2H), | |
| | 2.80-2.60 (m, 4H), 2.56-2.52 (m, 2H), 2.48-2.36 (m, 3H), 2.20-2.04 | |
| | (m, 2H), 2.00-1.92 (m, 1H), 1.76-1.64 (m, 4H), 1.44 (d, J = 6.8 Hz, | |
| | 6H) | |
| 414 | ¹H NMR (400 MHz, DMSO-d6) δ 10.96 (s, 1H), 9.96 (s, 1H), 8.80- | C |
| | 8.76 (m, 1H), 8.68 (d, J = 8.4 Hz, 1H), 8.48-8.40 (m, 1H), 7.80 (s, 1H), | |
| | 7.76 (d, J = 8.8 Hz, 1H), 7.72 (d, J = 8.4 Hz, 1H), 7.56-7.52 (m, 1H), | |
| | 7.48 (d, J = 8.4 Hz, 1H), 6.96-6.92 (m, 1H), 6.92 (d, J = 9.2 Hz, 1H), | |
| | 6.88 (d, J = 8.8 Hz, 1H), 5.12-5.02 (m, 1H), 4.36 (s, 2H), 4.28 (d, J = | |
| | 17.2 Hz, 1H), 4.12 (d, J = 17.2 Hz, 1H), 3.92 (d, J = 11.2 Hz, 1H), 3.88- | |
| | 3.76 (m, 1H), 3.72 (d, J = 9.2 Hz, 1H), 3.68-3.60 (m, 4H), 3.40-3.32 | |
| | (m, 2H), 3.02 (d, J = 10.4 Hz, 1H), 2.92-2.84 (m, 2H), 2.76-2.72 (m, | |
| | 1H), 2.72-2.68 (m, 1H), 2.56 (d, J = 4.0 Hz, 4H), 2.52 (s, 4H), 2.40- | |
| | 2.32 (m, 1H), 2.00-1.92 (m, 1H) | |
| 415 | ¹H NMR (400 MHz, DMSO-d6) δ 11.02-10.96 (m, 1H), 9.96 (s, 1H), | C |
| | 8.80 (s, 1H), 8.68 (d, J = 8.4 Hz, 1H), 8.48-8.40 (m, 1H), 8.16 (s, 1H), | |
| | 7.80 (s, 1H), 7.72 (d, J = 8.8 Hz, 1H), 7.64 (d, J = 8.0 Hz, 1H), 7.52 (dd, | |
| | J = 2.4, 10.0 Hz, 1H), 7.48 (d, J = 8.8 Hz, 1H), 7.28 (d, J = 8.4 Hz, 1H), | |
| | 6.96 (m, 7.6 Hz, 1H), 6.88 (d, J = 8.4 Hz, 1H), 5.12 (dd, J = 5.2, 13.2 | |
| | Hz, 1H), 4.48-4.36 (m, 3H), 4.28-4.20 (m, 1H), 3.92 (d, J = 10.8 Hz, | |
| | 1H), 3.88-3.80 (m, 1H), 3.76-3.68 (m, 1H), 3.56 (t, J = 4.8 Hz, 2H), | |
| | 3.06 (s, 3H), 3.02 (d, J = 11.2 Hz, 1H), 2.96-2.84 (m, 2H), 2.80-2.76 | |
| | (m, 1H), 2.72-2.68 (m, 2H), 2.64-2.56 (m, 4H), 2.48-2.36 (m, 2H), | |
| | 2.32-2.24 (m, 2H), 2.16 (s, 1H), 2.00-1.96 (m, 1H) | |
| 416 | ¹H NMR (400 MHz, DMSO-d6) δ 11.11 (s, 1H), 9.96 (s, 1H), 8.82 (s, | C |
| | 1H), 8.68 (d, J = 8.8 Hz, 1H), 8.45 (dd, J = 6.0, 7.2 Hz, 1H), 7.82 (s, | |
| | 1H), 7.71 (d, J = 8.8 Hz, 1H), 7.57-7.42 (m, 2H), 6.96 (dt, J = 2.4, 7.6 | |
| | Hz, 1H), 6.91-6.83 (m, 2H), 6.82-6.74 (m, 1H), 5.43-5.26 (m, 1H), | |
| | 4.76 (td, J = 6.4, 13.6 Hz, 1H), 4.39 (s, 2H), 3.91 (d, J = 11.2 Hz, 1H), | |
| | 3.74-3.60 (m, 2H), 3.40-3.36 (m, 3H), 2.97 (d, J = 9.2 Hz, 5H), 2.86 | |
| | (d, J = 11.2 Hz, 2H), 2.80-2.55 (m, 7H), 2.54 (s, 3H), 2.05-1.95 (m, | |
| | 1H), 1.80-1.63 (m, 2H), 1.41 (d, J = 6.8 Hz, 6H) | |
| 417 | ¹H NMR (400 MHz, DMSO-d6) δ 11.12 (s, 1H), 9.97 (s, 1H), 8.82 (s, | C |
| | 1H), 8.68 (d, J = 8.8 Hz, 1H), 8.44 (dd, J = 6.4, 7.2 Hz, 1H), 7.82 (s, | |
| | 1H), 7.71 (d, J = 8.5 Hz, 1H), 7.57-7.45 (m, 2H), 6.97 (dt, J = 2.6, 7.4 | |
| | Hz, 1H), 6.92-6.76 (m, 3H), 5.41-5.27 (m, 1H), 4.83-4.72 (m, 1H), | |
| | 4.39 (s, 2H), 3.92 (d, J = 10.0 Hz, 1H), 3.73-3.64 (m, 2H), 3.40-3.37 | |
| | (m, 3H), 3.11-2.94 (m, 5H), 2.92-2.81 (m, 3H), 2.80-2.71 (m, 2H), | |
| | 2.70-2.58 (m, 4H), 2.54 (s, 3H), 2.04-1.96 (m, 1H), 1.88-1.65 (m, | |
| | 2H), 1.41 (d, J = 6.8 Hz, 6H) | |
| 418 | ¹H NMR (400 MHz, DMSO-d6) δ 0.91 (s, 2H), 1.01-1.20 (m, 2H), | C |
| | 1.63-2.09 (m, 4H), 2.51-3.12 (m, 15H), 3.14-3.27 (m, 3H), 3.69 (d, | |
| | J = 8.24 Hz, 4H), 3.86-4.00 (m, 1H), 4.39 (s, 2H), 5.30 (dd, J = 12.24, 4.88 | |
| | Hz, 1H), 6.80-7.09 (m, 5H), 7.43-7.57 (m, 2H), 7.71 (d, J = 8.63 Hz, 1H), | |
| | 7.82 (s, 1H), 8.44 (dd, J = 7.24, 6.13 Hz, 1H), 8.68 (d, J = 8.48 Hz, 1H), | |
| | 8.82 (s, 1H), 9.98 (s, 1H), 11.09 (s, 1H) | |
| 419 | ¹H NMR (400 MHz, DMSO-d6) δ 10.96 (s, 1H), 10.07-9.91 (m, 1H), | C |
| | 8.81 (s, 1H), 8.67 (d, J = 8.4 Hz, 1H), 8.50-8.33 (m, 1H), 7.95-7.78 | |
| | (m, 2H), 7.71 (d, J = 8.4 Hz, 1H), 7.60-7.41 (m, 2H), 7.11-6.93 (m, | |
| | 2H), 6.92-6.84 (m, 1H), 5.15-5.03 (m, 1H), 4.63-4.53 (m, 1H), 4.42- | |
| | 4.36 (m, 2H), 4.35-4.25 (m, 1H), 4.19-4.10 (m, 1H), 3.96-3.89 (m, | |
| | 1H), 3.74-3.52 (m, 5H), 3.19-3.03 (m, 2H), 3.02-2.82 (m, 4H), 2.80- | |
| | 2.55 (m, 8H), 2.45-2.28 (m, 2H), 2.06-1.77 (m, 3H) | |
| 420 | ¹H NMR (400 MHz, DMSO-d6) δ 0.88-0.96 (m, 2H), 0.99-1.15 (m, | C |
| | 2H), 1.74-2.06 (m, 3H), 2.55-3.12 (m, 13H), 3.15-3.32 (m, 5H), 3.35- | |
| | 3.39 (m, 3H), 3.63-3.76 (m, 2H), 3.86-3.97 (m, 1H), 4.31-4.47 (m, 2H), | |
| | 5.30 (dd, J = 12.88, 4.88 Hz, 1H), 6.83-7.07 (m, 5H), 7.41-7.58 (m, 2H), | |
| | 7.71 (d, J = 8.56 Hz, 1H), 7.82 (s, 1H), 8.35-8.48 (m, 1H), 8.68 (d, J-8.64 | |
| | Hz, 1H), 8.82 (s, 1H), 9.97 (s, 1H), 11.08 (s, 1H) | |
| 421 | ¹H NMR (400 MHz, DMSO-d6) δ 10.97 (s, 1H), 9.98 (s, 1H), 8.82 (s, | C |
| | 1H), 8.68 (d, J = 8.8 Hz, 1H), 8.49-8.39 (m, 1H), 7.95-7.77 (m, 2H), | |
| | 7.71 (d, J = 8.8 Hz, 1H), 7.58-7.44 (m, 2H), 7.10-6.92 (m, 2H), 6.87 | |
| | (d, J = 8.4 Hz, 1H), 5.09 (dd, J = 5.2, 13.2 Hz, 1H), 4.69-4.47 (m, 1H), | |
| | 4.44-4.24 (m, 3H), 4.13 (d, J = 17.6 Hz, 1H), 3.92 (d, J = 11.2 Hz, 1H), | |
| | 3.81-3.36 (m, 6H), 3.24-2.83 (m, 6H), 2.81-2.64 (m, 2H), 2.63-2.51 | |
| | (m, 4H), 2.47-2.29 (m, 3H), 2.03-1.69 (m, 3H) | |
| 422 | ¹H NMR (400 MHz, DMSO-d6) δ 10.96 (s, 1H), 9.96 (s, 1H), 8.80 (s, | C |
| | 1H), 8.68 (d, J = 8.4 Hz, 1H), 8.44 (dd, J = 6.4, 7.2 Hz, 1H), 7.80 (s, | |
| | 1H), 7.72-7.68 (m, 1H), 7.64-7.60 (m, 1H), 7.52 (dd, J = 2.8, 10.0 Hz, | |
| | 1H), 7.48 (d, J = 8.8 Hz, 1H), 7.28 (d, J = 8.4 Hz, 1H), 6.96 (dt, J = 2.8, | |
| | 7.6 Hz, 1H), 6.84 (d, J = 8.4 Hz, 1H), 5.16-5.02 (m, 1H), 4.44-4.36 | |
| | (m, 3H), 4.32-4.20 (m, 1H), 3.92 (d, J = 10.8 Hz, 1H), 3.72-3.60 (m, | |
| | 2H), 3.36-3.32 (m, 2H), 3.20-3.06 (m, 4H), 3.02-2.64 (m, 5H), 2.60 | |
| | -2.56 (m, 4H), 2.52 (d, J = 5.2 Hz, 2H), 2.44 (d, J = 4.0 Hz, 2H), 2.00- | |
| | 1.96 (m, 1H), 1.678 (q, J = 6.8 Hz, 2H), 1.24 (s, 1H) | |
| 423 | ¹H NMR (400 MHz, DMSO-d6) δ 10.98 (s, 1H), 9.97 (s, 1H), 8.80 (s, | C |
| | 1H), 8.74 (d, J = 8.4Hz, 1H), 8.48-8.40 (m, 1H), 7.82 (s, 1H), 7.71 (d, J = | |
| | 8.4 Hz, 1H), 7.66 (d, J = 8.0 Hz, 1H), 7.56-7.49 (m, 2H), 7.29 (d, J = | |
| | 8.0 Hz, 1H), 7.00-6.92 (m, 1H), 6.82 (d, J = 8.4 Hz, 1H), 5.10 (dd, J = | |
| | 4.8, 13.2 Hz, 1H), 4.45-4.35 (m, 3H), 4.26 (d, J = 17.6 Hz, 1H), 3.29 | |
| | (s, 3H), 3.12 (s, 4H), 2.63-2.54 (m, 5H), 2.45-2.38 (m, 2H), 2.29- | |
| | 2.18 (m, 2H), 2.03-1.90 (m, 3H), 1.83-1.73 (m, 2H), 1.71-1.57 (m, | |
| | 1H), 1.50-1.39 (m, 2H), 1.23 (s, 1H), 1.17-1.00 (m, 2H) | |
| 424 | ¹H NMR (400 MHz, DMSO-d6) δ 10.99 (s, 1H), 10.05 (s, 1H), 8.82 (s, | C |
| | 1H), 8.76 (d, J = 8.4 Hz, 1H), 8.48-8.43 (m, 1H), 7.82 (s, 1H), 7.73 (d, | |
| | J = 8.4 Hz, 1H), 7.66 (d, J = 8.0 Hz, 1H), 7.52 (dd, J = 2.8, 10.0 Hz, | |
| | 1H), 7.39 (d, J = 8.4 Hz, 1H), 7.30 (d, J = 8.0 Hz, 1H), 7.00-6.93 (m, | |
| | 1H), 6.80 (d, J = 8.4 Hz, 1H), 5.61 (s, 1H), 5.10 (dd, J = 4.8, 13.2 Hz, | |
| | 1H), 4.46-4.37 (m, 3H), 4.30-4.22 (m, 1H), 3.13 (s, 4H), 2.91 (s, 1H), | |
| | 2.61 (s, 5H), 2.45 (s, 3H), 2.37-2.25 (m, 5H), 2.04-1.80 (m, 5H), | |
| | 1.47-1.34 (m, 1H) | |
| 425 | ¹H NMR (400 MHz, DMSO-d6) δ 10.98 (s, 1H), 9.91 (s, 1H), 8.77 (s, | C |
| | 1H), 8.64 (d, J = 8.8 Hz, 1H), 8.46-8.39 (m, 1H), 8.19 (s, 1H), 7.80 (s, | |
| | 1H), 7.70 (d, J = 8.4 Hz, 1H), 7.51 (dd, J = 2.4, 10.0 Hz, 1H), 7.47- | |
| | 7.41 (m, 2H), 7.31 (d, J = 7.2 Hz, 1H), 7.17 (d, J = 8.0 Hz, 1H), 6.96 (dt, | |
| | J = 2.4, 7.2 Hz, 1H), 6.84 (d, J = 8.8 Hz, 1H), 5.11 (dd, J = 5.2, 13.2 Hz, | |
| | 1H), 4.49-4.42 (m, 1H), 4.37 (s, 2H), 4.35-4.27 (m, 1H), 3.15-3.05 | |
| | (m, 5H), 3.01 (d, J = 11.2 Hz, 3H), 2.93-2.87 (m, 1H), 2.64-2.58 (m, | |
| | 3H), 2.54 (s, 4H), 2.45 (s, 3H), 2.28 (d, J = 7.2 Hz, 2H), 2.04-1.97 (m, | |
| | 1H), 1.84 (d, J = 11.6 Hz, 2H), 1.72-1.61 (m, 1H), 1.31 (q, J = 10.4 Hz, | |
| | 2H) | |
| 426 | ¹H NMR (400 MHz, DMSO-d6) δ 10.96 (s, 1H), 10.21 (s, 1H), 8.90 (s, | C |
| | 1H), 8.59 (d, J = 8.8 Hz, 1H), 8.41 (dd, J = 5.6, 7.2 Hz, 1H), 7.89 (s, | |
| | 1H), 7.82 (t, J = 7.6 Hz, 1H), 7.73 (d, J = 8.4 Hz, 1H), 7.59 (dd, J = 2.0, | |
| | 9.6 Hz, 1H), 7.17-7.10 (m, 2H), 7.05 (dt, J = 2.8, 7.2 Hz, 1H), 6.85 (d, | |
| | J = 7.6 Hz, 1H), 5.02 (dd, J = 5.2, 13.2 Hz, 1H), 4.42-4.35 (m, 4H), | |
| | 4.22 (d, J = 17.2 Hz, 1H), 3.59 (d, J = 10.4 Hz, 2H), 3.35-3.28 (m, 2H), | |
| | 3.16-3.05 (m, 2H), 2.93-2.85 (m, 1H), 2.75-2.67 (m, 2H), 2.64-2.55 | |
| | (m, 2H), 2.53 (s, 3H), 2.46-2.31 (m, 2H), 1.98-1.86 (m, 3H), 1.76- | |
| | 1.70 (m, 2H), 1.51-1.35 (m, 3H), 1.30-1.15 (m, 4H) | |
| 427 | ¹H NMR (400 MHz, DMSO-d6) δ 10.82 (s, 1H), 9.93 (s, 1H), 8.79 (s, | C |
| | 1H), 8.65 (d, J = 8.4 Hz, 1H), 8.47-8.41 (m, 1H), 8.13 (s, 1H), 7.98 (s, | |
| | 1H), 7.82 (s, 1H), 7.70 (d, J = 8.4 Hz, 1H), 7.52 (dd, J = 2.4, 10.0 Hz, | |
| | 1H), 7.45 (d, J = 8.4 Hz, 2H), 6.96 (dt, J = 2.4, 7.2 Hz, 1H), 6.85 (d, J = | |
| | 8.4 Hz, 2H), 4.38 (s, 2H), 3.75 (dd, J = 4.8, 11.6 Hz, 1H), 3.57-3.37 | |
| | (m, 3H), 3.03 (d, J = 10.8 Hz, 3H), 2.71-2.60 (m, 4H), 2.54 (s, 5H), | |
| | 2.45 (s, 3H), 2.27-2.12 (m, 2H), 1.99-1.94 (m, 1H), 1.86 (d, J = 10.8 | |
| | Hz, 2H), 1.38-1.21 (m, 3H) | |
| 428 | ¹H NMR (400 MHz, DMSO-d6) δ 11.00 (s, 1H), 9.98 (s, 1H), 8.81 (s, | C |
| | 1H), 8.68 (d, J = 8.4 Hz, 1H), 8.44 (dd, J = 6.0, 7.2 Hz, 1H), 7.82 (s, | |
| | 1H), 7.71 (d, J = 8.8 Hz, 1H), 7.62-7.56 (m, 1H), 7.52 (dd, J = 2.8, 10.4 | |
| | Hz, 2H), 7.48 (s, 1H), 7.00-6.94 (m, 1H), 6.88 (d, J = 8.4 Hz, 1H), 5.11 | |
| | (dd, J = 5.2, 13.2 Hz, 1H), 4.55 (d, J = 17.6 Hz, 1H), 4.39 (s, 2H), 4.35 | |
| | (s, 1H), 3.96 (d, J = 10.8 Hz, 2H), 3.80-3.73 (m, 1H), 3.50-3.37 (m, | |
| | 4H), 3.11-3.04 (m, 1H), 3.03-2.94 (m, 2H), 2.92-2.85 (m, 2H), 2.83- | |
| | 2.71 (m, 2H), 2.60 (d, J = 17.6 Hz, 2H), 2.56-2.52 (m, 2H), 2.48- | |
| | 2.36 (m, 2H), 2.05-1.93 (m, 2H), 1.91-1.76 (m, 3H) | |
| 429 | ¹H NMR (400 MHz, DMSO-d6) δ 11.07 (s, 1H), 9.92 (s, 1H), 8.79 (s, | C |
| | 1H), 8.65 (d, J = 8.4 Hz, 1H), 8.48-8.40 (m, 1H), 7.81 (s, 1H), 7.70 (d, | |
| | J = 8.4 Hz, 1H), 7.52 (dd, J = 2.4, 10.0 Hz, 1H), 7.44 (d, J = 8.4 Hz, | |
| | 1H), 6.99-6.91 (m, 2H), 6.88-6.78 (m, 2H), 6.68-6.58 (m, 1H), 5.29 | |
| | (dd, J = 5.2, 12.4 Hz, 1H), 4.38 (s, 2H), 3.32-3.30 (m, 4H), 3.10 (s, | |
| | 3H), 3.01 (d, J = 10.8 Hz, 2H), 2.89 (s, 1H), 2.71-2.61 (m, 3H), 2.60- | |
| | 2.53 (m, 4H), 2.45 (s, 3H), 2.27 (d, J = 6.4 Hz, 1H), 2.02-1.95 (m, 1H), | |
| | 1.84 (d, J = 11.2 Hz, 2H), 1.73-1.63 (m, 1H), 1.39-1.21 (m, 4H) | |
| 430 | ¹H NMR (400 MHz, DMSO-d6) δ 11.04-10.96 (m, 1H), 10.00-9.92 | C |
| | (m, 1H), 8.80 (s, 1H), 8.68 (d, J = 8.8 Hz, 1H), 8.44 (dd, J = 6.0, 7.2 Hz, | |
| | 1H), 8.20 (s, 1H), 8.08-8.00 (m, 1H), 7.80 (s, 1H), 7.72 (d, J = 8.8 Hz, | |
| | 1H), 7.52 (dd, J = 2.4, 10.1 Hz, 1H), 7.48 (t, J = 8.8 Hz, 2H), 7.00-6.92 | |
| | (m, 1H), 6.88 (d, J = 8.4 Hz, 1H), 5.16 (dd, J = 5.2, 13.2 Hz, 1H), 4.48 | |
| | (d, J = 17.6 Hz, 1H), 4.40 (s, 2H), 4.32-4.28 (m, 1H), 3.92 (d, J = 10.8 | |
| | Hz, 1H), 3.84-3.76 (m, 1H), 3.72-3.68 (m, 1H), 3.06 (d, J = 9.6 Hz, | |
| | 1H), 3.02-2.96 (m, 2H), 2.92-2.84 (m, 2H), 2.80-2.72 (m, 2H), 2.64- | |
| | 2.52 (m, 4H), 2.48 (s, 3H), 2.44-2.36 (m, 1H), 2.24-2.08 (m, 2H), | |
| | 2.04-1.96 (m, 1H), 1.88-1.68 (m, 4H) | |
| 431 | ¹H NMR (400 MHz, DMSO-d6) δ 11.08 (s, 1H), 9.96 (s, 1H), 8.84 (s, | C |
| | 1H), 8.68 (d, J = 8.4 Hz, 1H), 8.56 (d, J = 7.2 Hz, 1H), 8.44 (s, 1H), 8.16 | |
| | (s, 1H), 7.76 (d, J = 8.8 Hz, 1H), 7.44 (d, J = 8.8 Hz, 1H), 7.20 (dd, J = | |
| | 1.6, 7.2 Hz, 1H), 6.96 (d, J = 8.8 Hz, 1H), 6.88-6.80 (m, 2H), 6.64 (d, J = | |
| | 8.4 Hz, 1H), 5.36-5.24 (m, 1H), 4.40 (s, 2H), 3.32 (s, 3H), 3.16- | |
| | 3.02 (m, 6H), 2.96-2.84 (m, 1H), 2.76-2.60 (m, 8H), 2.48 (s, 3H), | |
| | 2.44-2.36 (m, 1H), 2.04-1.88 (m, 3H), 1.72-1.56 (m, 2H) | |
| 432 | ¹H NMR (400 MHz, DMSO-d6) δ 10.98 (s, 1H), 9.92 (s, 1H), 8.78 (s, | C |
| | 1H), 8.65 (d, J = 8.4 Hz, 1H), 8.47-8.41 (m, 1H), 7.81 (s, 1H), 7.68 | |
| | (dd, J = 8.4, 14.8 Hz, 2H), 7.51 (dd, J = 2.4, 10.0 Hz, 1H), 7.44 (d, J = | |
| | 8.8 Hz, 1H), 7.29 (d, J = 8.0 Hz, 1H), 6.96 (dt, J = 2.4, 7.6 Hz, 1H), 6.84 | |
| | (d, J = 8.4 Hz, 1H), 5.10 (dd, J = 5.2, 13.2 Hz, 1H), 4.45-4.39 (m, 1H), | |
| | 4.38 (s, 2H), 4.30-4.23 (m, 1H), 3.12 (s, 4H), 3.02 (d, J = 11.2 Hz, 2H), | |
| | 2.96-2.86 (m, 1H), 2.68-2.53 (m, 7H), 2.45 (s, 4H), 2.36-2.22 (m, | |
| | 2H), 2.04-1.95 (m, 1H), 1.90-1.80 (m, 2H), 1.76-1.62 (m, 1H), | |
| | 1.38-1.27 (m, 2H) | |
| 433 | ¹H NMR (400 MHz, DMSO-d6) δ 11.06 (s, 1H), 9.96 (s, 1H), 8.80 (s, | C |
| | 1H), 8.68 (d, J = 8.4 Hz, 1H), 8.44 (dd, J = 6.0, 7.6 Hz, 1H), 7.80 (s, | |
| | 1H), 7.72 (d, J = 8.4 Hz, 1H), 7.56-7.44 (m, 2H), 7.00-6.84 (m, 4H), | |
| | 6.60 (dd, J = 2.0, 8.8 Hz, 1H), 5.28 (dd, J = 5.6, 12.8 Hz, 1H), 4.56 (m, | |
| | J = 6.8 Hz, 1H), 4.36 (s, 2H), 3.96-3.88 (m, 1H), 3.84-3.80 (m, 1H), | |
| | 3.76-3.64 (m, 1H), 3.06 (s, 4H), 3.02 (d, J = 11.2 Hz, 1H), 2.92-2.84 | |
| | (m, 2H), 2.80-2.68 (m, 2H), 2.64-2.56 (m, 6H), 2.52-2.48 (m, 2H), | |
| | 2.40 (s, 3H), 2.00-1.92 (m, 1H), 1.44 (d, J = 6.8 Hz, 6H) | |
| 434 | ¹H NMR (400 MHz, DMSO-d6) δ 11.04-10.88 (m, 1H), 9.96 (s, 1H), | C |
| | 8.80 (s, 1H), 8.64 (d, J = 8.4 Hz, 1H), 8.44 (dd, J = 6.0, 7.2 Hz, 1H), | |
| | 7.80 (s, 1H), 7.72-7.68 (m, 1H), 7.64 (d, J = 8.0 Hz, 1H), 7.52 (dd, J = | |
| | 2.4, 10.0 Hz, 1H), 7.48-7.44 (m, 1H), 7.28 (d, J = 8.0 Hz, 1H), 6.96 (dt, | |
| | J = 2.8, 7.6 Hz, 1H), 6.88 (d, J = 8.8 Hz, 1H), 5.06 (dd, J = 5.2, 13.2 Hz, | |
| | 1H), 4.48-4.32 (m, 3H), 4.28-4.20 (m, 1H), 3.92 (d, J = 11.2 Hz, 1H), | |
| | 3.72-3.64 (m, 2H), 3.36-3.32 (m, 2H), 3.16-3.06 (m, 4H), 3.02-2.68 | |
| | (m, 5H), 2.64-2.52 (m, 6H), 2.48 (s, 3H), 2.04-1.92 (m, 1H), 1.72- | |
| | 1.60 (m, 2H) | |
| 435 | ¹H NMR (400 MHz, DMSO-d6) δ 10.97 (s, 1H), 9.96 (s, 1H), 8.81 (s, | D |
| | 1H), 8.71 (d, J = 8.4 Hz, 1H), 8.48-8.41 (m, 1H), 7.82 (s, 1H), 7.71 (d, | |
| | J = 8.4 Hz, 1H), 7.55-7.48 (m, 2H), 7.37 (d, J = 8.0 Hz, 1H), 6.96 (dt, J = | |
| | 2.4, 7.2 Hz, 1H), 6.79 (d, J = 8.4 Hz, 1H), 6.58 (t, J = 8.0 Hz, 1H), | |
| | 5.04 (dd, J = 4.8, 13.2 Hz, 1H), 4.47-4.34 (m, 3H), 4.25 (d, J = 16.8 | |
| | Hz, 1H), 4.13 (s, 2H), 3.86 (s, 2H), 3.00-2.81 (m, 5H), 2.67-2.57 (m, | |
| | 4H), 2.44-2.32 (m, 2H), 2.23-2.14 (m, 2H), 1.99-1.91 (m, 1H), 1.77- | |
| | 1.61 (m, 4H), 1.51-1.38 (m, 2H), 0.51 (s, 2H), 0.41 (s, 2H) | |
| 436 | ¹H NMR (400 MHz, DMSO-d6) δ 10.98 (s, 1H), 10.08 (s, 1H), 8.88- | E |
| | 8.73 (m, 2H), 8.45 (dd, J = 5.8, 7.2 Hz, 1H), 8.13 (s, 1H), 7.83 (s, 1H), | |
| | 7.74 (d, J = 8.4 Hz, 1H), 7.62 (d, J = 8.4 Hz, 1H), 7.53 (dd, J = 2.6, 10.0 | |
| | Hz, 1H), 7.46-7.39 (m, 1H), 7.30 (d, J = 7.2 Hz, 1H), 7.16 (d, J = 8.0 | |
| | Hz, 1H), 6.97 (dt, J = 2.7, 7.6 Hz, 1H), 6.83 (d, J = 8.4 Hz, 1H), 5.12 | |
| | (dd, J = 5.1, 13.2 Hz, 1H), 4.51-4.38 (m, 5H), 4.32-4.25 (m, 1H), 3.37 | |
| | (br d, J = 14.8 Hz, 4H), 3.01-2.81 (m, 3H), 2.75 (br d, J = 10.8 Hz, | |
| | 2H), 2.67-2.54 (m, 2H), 2.47-2.36 (m, 3H), 2.04-1.93 (m, 3H), 1.90- | |
| | 1.72 (m, 4H), 1.70-1.48 (m, 2H), 1.41-1.19 (m, 3H) | |
| 437 | ¹H NMR (400 MHz, DMSO-d6) δ 11.06 (s, 1H), 10.08 (s, 1H), 8.83 (s, | E |
| | 1H), 8.76 (d, J = 8.5 Hz, 1H), 8.48-8.40 (m, 1H), 8.13 (s, 1H), 7.83 (s, | |
| | 1H), 7.74 (d, J = 8.6 Hz, 1H), 7.62 (br d, J = 8.3 Hz, 1H), 7.52 (dd, J = | |
| | 2.7, 10.1 Hz, 1H), 7.01-6.89 (m, 2H), 6.87-6.77 (m, 2H), 6.64 (dd, J = | |
| | 2.1, 8.6 Hz, 1H), 5.33-5.25 (m, 1H), 4.49 (s, 2H), 4.40 (s, 2H), 3.66-3.55 | |
| | (m, 3H), 3.49-3.42 (m, 3H), 3.19 (br dd, J = 5.1, 7.8 Hz, 3H), | |
| | 2.94-2.78 (m, 3H), 2.65-2.57 (m, 4H), 2.43-2.38 (m, 3H), 2.08-1.92 (m, | |
| | 4H), 1.86-1.60 (m, 5H), 1.37-1.19 (m, 4H) | |
| 438 | ¹H NMR (400 MHz, DMSO-d6) δ 11.07 (s, 1H), 9.93 (s, 1H), 8.79 (s, | H |
| | 1H), 8.66 (d, J = 8.4 Hz, 1H), 7.81 (s, 1H), 7.70 (d, J = 8.8 Hz, 1H), 7.52 | |
| | (dd, J = 2.4, 10.0 Hz, 1H), 7.44 (d, J = 8.8 Hz, 1H), 7.05-6.90 (m, 2H), | |
| | 6.87-6.77 (m, 2H), 6.64 (dd, J = 1.6, 8.6 Hz, 1H), 5.29 (dd, J = 5.2, | |
| | 12.8 Hz, 1H), 4.38 (s, 2H), 3.63 (s, 2H), 3.45 (d, J = 12.4 Hz, 2H), 3.31 | |
| | (s, 3H), 3.03-2.94 (m, 2H), 2.92-2.81 (m, 3H), 2.78-2.55 (m, 5H), | |
| | 2.46 (s, 3H), 2.04-1.89 (m, 5H), 1.70-1.54 (m, 4H) | |
| 439 | ¹H NMR (400 MHz, DMSO-d6) δ 11.09 (s, 1H), 9.93 (s, 1H), 8.79 (s, | H |
| | 1H), 8.66 (d, J = 8.8 Hz, 1H), 8.45 (t, J = 6.4 Hz, 1H), 7.82 (s, 1H), 7.71 | |
| | (d, J = 8.4 Hz, 1H), 7.52 (dd, J = 2.4, 10.0 Hz, 1H), 7.45 (d, J = 8.8 Hz, | |
| | 1H), 7.01-6.93 (m, 2H), 6.93-6.80 (m, 3H), 5.35 (dd, J = 4.8, 12.4 Hz, | |
| | 1H), 4.38 (s, 2H), 3.64 (s, 5H), 3.10 (s, 2H), 3.00 (d, J = 10.4 Hz, 2H), | |
| | 2.95-2.83 (m, 2H), 2.81-2.75 (m, 1H), 2.74-2.66 (m, 4H), 2.65-2.57 | |
| | (m, 2H), 2.09-1.92 (m, 5H), 1.90-1.81 (m, 1H), 1.64 (d, J = 5.2 Hz, | |
| | 4H) | |
| 440 | ¹H NMR (400 MHz, DMSO-d6) δ 11.16-11.02 (m, 1H), 9.76 (s, 1H), | I |
| | 8.76 (s, 1H), 8.52 (d, J = 8.8 Hz, 1H), 8.44 (dd, J = 6.0, 7.6 Hz, 1H), | |
| | 7.80 (s, 1H), 7.68 (d, J = 8.4 Hz, 1H), 7.52 (dd, J = 2.4, 10.0 Hz, 1H), | |
| | 7.00-6.92 (m, 3H), 6.88 (d, J = 2.0 Hz, 1H), 6.80 (d, J = 8.4 Hz, 1H), | |
| | 6.68 (dd, J = 2.0, 8.4 Hz, 1H), 5.36-5.28 (m, 1H), 4.36 (s, 2H), 3.68 (s, | |
| | 4H), 3.32 (s, 3H), 3.12 (s, 4H), 2.96-2.84 (m, 1H), 2.68-2.56 (m, 2H), | |
| | 2.40 (s, 3H), 2.00-1.96 (m, 1H), 1.92 (s, 4H) | |
| 441 | ¹H NMR (400 MHz, DMSO-d6) δ 10.93 (s, 1H), 9.97 (s, 1H), 9.90- | I |
| | 9.75 (m, 1H), 8.86-8.76 (m, 1H), 8.73-8.63 (m, 1H), 8.45 (t, J = 6.8 | |
| | Hz, 1H), 7.82 (s, 1H), 7.72 (d, J = 8.4 Hz, 1H), 7.61 (t, J = 8.8 Hz, 1H), | |
| | 7.56-7.49 (m, 2H), 7.01-6.76 (m, 4H), 4.39 (s, 2H), 3.75 (t, J = 7.2 | |
| | Hz, 1H), 3.32-3.28 (m, 4H), 2.98 (s, 4H), 2.60-2.55 (m, 2H), 2.52- | |
| | 2.51 (m, 3H), 2.20-2.10 (m, 2H) | |
| 442 | ¹H NMR (400 MHz, DMSO-d6) δ 10.84 (s, 1H), 9.96 (s, 1H), 8.80 (s, | I |
| | 1H), 8.68 (d, J = 8.4 Hz, 1H), 8.48-8.40 (m, 1H), 8.00 (d, J = 2.4 Hz, | |
| | 1H), 7.80 (s, 1H), 7.72 (d, J = 8.8 Hz, 1H), 7.56-7.48 (m, 2H), 7.44- | |
| | 7.40 (m, 1H), 6.96 (dt, J = 2.8, 7.6 Hz, 1H), 6.92-6.80 (m, 2H), 4.44- | |
| | 4.36 (m, 2H), 3.76 (dd, J = 4.8, 12.0 Hz, 1H), 3.64 (s, 4H), 3.02-2.84 | |
| | (m, 4H), 2.76-2.64 (m, 1H), 2.56 (s, 1H), 2.52 (s, 3H), 2.20 (dq, J = | |
| | 4.0, 12.4 Hz, 1H), 2.04-1.96 (m, 1H) | |
| 443 | ¹H NMR (400 MHz, DMSO-d6) δ 11.11 (d, J = 2.4 Hz, 1H), 9.99 (s, | I |
| | 1H), 8.81 (s, 1H), 8.70 (d, J = 8.8 Hz, 1H), 8.49-8.43 (m, 1H), 7.82 (s, | |
| | 1H), 7.72 (d, J = 8.4 Hz, 1H), 7.60-7.49 (m, 2H), 7.03 (d, J = 4.4 Hz, | |
| | 2H), 7.00-6.91 (m, 2H), 6.88 (d, J = 8.4 Hz, 1H), 5.37 (dd, J = 4.8, 12.4 | |
| | Hz, 1H), 4.39 (s, 2H), 3.70 (s, 3H), 3.12 (s, 3H), 3.05 (s, 4H), 2.94- | |
| | 2.85 (m, 1H), 2.73-2.68 (m, 1H), 2.65-2.59 (m, 2H), 2.53 (s, 3H), | |
| | 2.05-1.97 (m, 1H) | |
| 444 | ¹H NMR (400 MHz, DMSO-d6) δ 11.08 (s, 1H), 9.97 (s, 1H), 8.81 (s, | I |
| | 1H), 8.69 (d, J = 8.4 Hz, 1H), 8.47-8.42 (m, 1H), 7.82 (s, 1H), 7.72 (d, | |
| | J = 8.4 Hz, 1H), 7.55-7.50 (m, 2H), 7.00-6.92 (m, 3H), 6.88 (d, J = | |
| | 8.4 Hz, 1H), 6.71 (dd, J = 2.0, 8.8 Hz, 1H), 5.31 (dd, J = 5.2, 12.8 Hz, | |
| | 1H), 4.39 (s, 2H), 3.38-3.35 (m, 1H), 3.30 (s, 1H), 3.27 (s, 4H), 3.02 | |
| | (d, J = 4.2 Hz, 4H), 2.95-2.85 (m, 1H), 2.76-2.58 (m, 3H), 2.51 (s, | |
| | 3H), 2.04-1.97 (m, 1H) | |
| 445 | ¹H NMR (400 MHz, DMSO-d6) δ 10.64 (s, 1H), 9.92 (s, 1H), 8.80 (s, | J |
| | 1H), 8.64 (d, J = 8.4 Hz, 1H), 8.44 (dd, J = 6.0, 7.6 Hz, 1H), 7.88-7.76 | |
| | (m, 2H), 7.72 (d, J = 8.8 Hz, 1H), 7.52 (dd, J = 2.4, 10.0 Hz, 1H), 7.48- | |
| | 7.40 (m, 2H), 6.96 (dt, J = 2.8, 7.6 Hz, 1H), 6.84-6.76 (m, 2H), 6.52 (d, | |
| | J = 7.6 Hz, 1H), 4.36 (s, 2H), 4.04-3.92 (m, 2H), 3.76 (t, J = 6.8 Hz, | |
| | 2H), 3.64-3.56 (m, 1H), 3.56-3.52 (m, 1H), 3.44 (s, 2H), 3.12-3.06 | |
| | (m, 2H), 3.02-2.92 (m, 2H), 2.88-2.76 (m, 2H), 2.68 (t, J = 9.6 Hz, | |
| | 2H), 2.44 (s, 3H), 1.92 (d, J = 7.2 Hz, 8H), 1.64-1.52 (m, 4H), 1.32- | |
| | 1.24 (m, 4H) | |
| 446 | ¹H NMR (400 MHz, DMSO-d6) δ 11.05 (s, 1H), 9.92 (s, 1H), 8.79 (s, | J |
| | 1H), 8.65 (d, J = 8.4 Hz, 1H), 8.48-8.37 (m, 1H), 7.81 (s, 1H), 7.70 (d, | |
| | J = 8.4 Hz, 1H), 7.52 (dd, J = 2.4, 10.0 Hz, 1H), 7.44 (d, J = 8.8 Hz, | |
| | 1H), 7.00-6.91 (m, 2H), 6.84 (d, J = 8.4 Hz, 1H), 6.64 (s, 1H), 5.00 | |
| | (dd, J = 5.2, 12.8 Hz, 1H), 4.38 (s, 2H), 4.09 (d, J = 12.4 Hz, 2H), 3.92 | |
| | (s, 3H), 3.04-2.94 (m, 4H), 2.90-2.81 (m, 2H), 2.65-2.61 (m, 2H), | |
| | 2.45 (s, 3H), 2.00-1.92 (m, 1H), 1.82-1.66 (m, 5H), 1.58-1.44 (m, | |
| | 1H), 1.39-1.13 (m, 7H) | |
| 447 | ¹H NMR (400 MHz, DMSO-d6) δ 11.04 (s, 1H), 9.98 (s, 1H), 8.79 (s, | J |
| | 1H), 8.73 (d, J = 8.8 Hz, 1H), 8.42 (t, J = 6.8 Hz, 1H), 8.26 (s, 1H), 7.81 | |
| | (s, 1H), 7.69 (d, J = 8.8 Hz, 1H), 7.56-7.48 (m, 2H), 7.02-6.89 (m, | |
| | 3H), 6.64 (s, 1H), 5.00 (dd, J = 5.2, 12.8 Hz, 1H), 4.37 (s, 2H), 4.08 (d, J = | |
| | 12.4 Hz, 2H), 3.92 (s, 3H), 3.54 (s, 2H), 3.21 (d, J = 10.4 Hz, 2H), | |
| | 2.98 (t, J = 12.0 Hz, 2H), 2.90-2.84 (m, 1H), 2.63 (d, J = 11.2 Hz, 2H), | |
| | 2.32 (s, 6H), 1.99-1.92 (m, 1H), 1.81-1.68 (m, 5H), 1.55-1.45 (m, | |
| | 1H), 1.38-1.09 (m, 7H) | |
| 448 | ¹H NMR (400 MHz, DMSO-d6) δ 10.99 (s, 1H), 10.12 (s, 1H), 8.85 (s, | K |
| | 1H), 8.80 (d, J = 8.8 Hz, 1H), 8.50-8.42 (m, 1H), 7.84 (s, 1H), 7.75 (d, | |
| | J = 8.4 Hz, 1H), 7.65 (d, J = 8.0 Hz, 1H), 7.53 (dd, J = 2.4, 10.0 Hz, | |
| | 1H), 7.49-7.44 (m, 2H), 7.28 (d, J = 8.4 Hz, 1H), 6.97 (dt, J = 2.4, 7.2 | |
| | Hz, 1H), 6.88 (d, J = 8.4 Hz, 1H), 5.10 (dd, J = 5.2, 13.2 Hz, 1H), 4.48- | |
| | 4.33 (m, 4H), 4.26 (d, J = 17.2 Hz, 1H), 4.08 (d, J = 6.8 Hz, 2H), 3.45- | |
| | 3.39 (m, 2H), 2.95-2.86 (m, 1H), 2.78-2.70 (m, 2H), 2.62-2.56 (m, | |
| | 2H), 2.40 (s, 3H), 2.21 (s, 3H), 1.99 (d, J = 6.4 Hz, 2H), 1.69 (d, J = | |
| | 11.2 Hz, 2H), 1.55-1.47 (m, 2H) | |
| 449 | ¹H NMR (400 MHz, DMSO-d6) δ 10.98 (s, 1H), 10.13 (s, 1H), 8.90- | K |
| | 8.72 (m, 2H), 8.46 (t, J = 6.4 Hz, 1H), 7.84 (s, 1H), 7.75 (d, J = 8.5 Hz, | |
| | 1H), 7.65 (d, J = 8.1 Hz, 1H), 7.55-7.49 (m, 1H), 7.41 (d, J = 8.1 Hz, | |
| | 1H), 7.28 (br d, J = 7.5 Hz, 1H), 7.01-6.93 (m, 1H), 6.88 (d, J = 8.4 Hz, | |
| | 1H), 5.10 (dd, J = 5.0, 13.4 Hz, 1H), 4.45-4.36 (m, 3H), 4.31-4.18 (m, | |
| | 1H), 3.99 (br d, J = 6.4 Hz, 2H), 3.43 (br d, J = 3.1 Hz, 3H), 2.96-2.54 | |
| | (m, 6H), 2.28 (s, 3H), 2.10 (s, 3H), 1.99 (s, 3H), 1.73-1.64 (m, 2H), | |
| | 1.54-1.45 (m, 2H) | |
| 450 | ¹H NMR (400 MHz, DMSO-d6) δ 10.87 (d, J = 12.0 Hz, 1H), 9.92 (s, | K |
| | 1H), 8.77 (s, 1H), 8.64 (d, J = 8.4 Hz, 1H), 8.46-8.40 (m, 1H), 7.81 (s, | |
| | 1H), 7.72-7.66 (m, 2H), 7.51 (dd, J = 2.4, 10.0 Hz, 1H), 7.44 (d, J = 8.8 | |
| | Hz, 1H), 6.96 (dt, J = 2.8, 7.6 Hz, 1H), 6.89-6.82 (m, 2H), 4.73-4.64 | |
| | (m, 1H), 4.53-4.40 (m, 3H), 4.37 (s, 2H), 3.03-2.91 (m, 4H), 2.68- | |
| | 2.58 (m, 4H), 2.54 (s, 1H), 2.45 (s, 3H), 2.02-1.95 (m, 1H), 1.77 (d, J = | |
| | 13.2 Hz, 5H), 1.55-1.47 (m, 1H), 1.39 (dd, J = 6.8, 14.4 Hz, 3H), 1.30 | |
| | (d, J = 9.2 Hz, 2H), 1.25-1.21 (m, 2H), 1.13-1.05 (m, 2H) | |
| 451 | ¹H NMR (400 MHz, DMSO-d6) δ 10.99 (s, 1H), 10.15 (s, 1H), 8.92 (s, | K |
| | 1H), 8.86 (d, J = 8.5 Hz, 1H), 8.76 (dd, J = 5.4, 7.3 Hz, 1H), 8.37 (s, | |
| | 1H), 8.07 (s, 1H), 8.03-7.94 (m, 1H), 7.81 (d, J = 8.6 Hz, 1H), 7.78- | |
| | 7.72 (m, 2H), 7.65 (d, J = 8.1 Hz, 1H), 7.52-7.44 (m, 1H), 7.27 (d, J = | |
| | 8.3 Hz, 1H), 6.94 (d, J = 8.5 Hz, 1H), 5.10 (dd, J = 4.9, 13.3 Hz, 1H), | |
| | 4.46-4.36 (m, 3H), 4.25 (d, J = 17.4 Hz, 1H), 4.15 (br d, J = 6.8 Hz, | |
| | 2H), 3.47-3.36 (m, 3H), 2.91 (br s, 1H), 2.78-2.70 (m, 2H), 2.62 (s, | |
| | 3H), 2.14-1.91 (m, 2H), 1.75-1.62 (m, 2H), 1.56-1.40 (m, 2H) | |
| 452 | ¹H NMR (400 MHz, DMSO-d6) δ 10.94 (s, 1H), 9.95 (s, 1H), 8.81 (s, | K |
| | 1H), 8.67 (d, J = 8.8 Hz, 1H), 8.62-8.55 (m, 1H), 8.08 (s, 1H), 7.80- | |
| | 7.68 (m, 3H), 7.45 (d, J = 8.4 Hz, 1H), 7.27-7.16 (m, 1H), 6.88 (dd, J = | |
| | 8.8, 15.8 Hz, 2H), 5.13-5.02 (m, 1H), 4.46-4.37 (m, 3H), 4.31-4.21 | |
| | (m, 1H), 4.09 (d, J = 17.2 Hz, 1H), 3.05-2.86 (m, 6H), 2.64-2.58 (m, | |
| | 2H), 2.45 (s, 3H), 2.40-2.28 (m, 2H), 1.99-1.90 (m, 1H), 1.78-1.70 | |
| | (m, 4H), 1.54-1.46 (m, 1H), 1.33-1.03 (m, 7H) | |
| 453 | ¹H NMR (400 MHz, DMSO-d6) δ 10.95 (s, 1H), 9.96 (s, 1H), 8.79 (s, | L |
| | 1H), 8.67 (d, J = 8.4 Hz, 1H), 8.44 (dd, J = 6.0, 7.2 Hz, 1H), 7.81 (s, | |
| | 1H), 7.71 (d, J = 8.4 Hz, 1H), 7.51 (dd, J = 2.4, 10.0 Hz, 1H), 7.46 (d, J = | |
| | 8.8 Hz, 1H), 7.37 (d, J = 8.0 Hz, 1H), 6.96 (dt, J = 2.8, 7.6 Hz, 1H), | |
| | 6.85 (d, J = 8.8 Hz, 1H), 6.59 (t, J = 8.0 Hz, 1H), 5.04 (dd, J = 5.2, 13.2 | |
| | Hz, 1H), 4.47-4.40 (m, 1H), 4.38 (s, 2H), 4.26 (d, J = 16.8 Hz, 1H), | |
| | 4.10 (s, 2H), 3.95 (s, 2H), 3.62 (s, 2H), 3.50 (s, 2H), 2.92-2.85 (m, | |
| | 1H), 2.79 (s, 4H), 2.63-2.53 (m, 2H), 2.51-2.50 (m, 3H), 2.43 (d, J = | |
| | 8.4 Hz, 5H), 2.00-1.91 (m, 1H) | |
| 454 | ¹H NMR (400 MHz, DMSO-d6) δ 10.96 (s, 1H), 9.88 (s, 1H), 8.78 (s, | L |
| | 1H), 8.57 (d, J = 8.5 Hz, 1H), 8.47-8.36 (m, 1H), 8.02 (d, J = 2.6 Hz, | |
| | 1H), 7.81 (s, 1H), 7.69 (d, J = 8.8 Hz, 1H), 7.50 (ddd, J = 2.7, 9.5, 17.4 | |
| | Hz, 2H), 7.37 (d, J = 8.3 Hz, 1H), 7.04-6.92 (m, 2H), 6.59 (t, J = 8.2 | |
| | Hz, 1H), 5.04 (dd, J = 5.0, 13.1 Hz, 1H), 4.49-4.19 (m, 4H), 4.11 (s, | |
| | 2H), 3.94 (s, 2H), 3.62 (br d, J = 1.4 Hz, 2H), 3.51 (br d, J = 2.1 Hz, | |
| | 2H), 3.39-3.34 (m, 1H), 3.07 (br s, 4H), 2.97-2.81 (m, 1H), 2.63- | |
| | 2.52 (m, 2H), 2.42 (br d, J = 8.4 Hz, 4H), 2.00-1.91 (m, 1H) | |
| 455 | ¹H NMR (400 MHz, DMSO-d6) δ 9.99 (s, 1H), 8.81 (s, 1H), 8.75 (d, J = | L |
| | 8.8 Hz, 1H), 8.57-8.44 (m, 3H), 7.88 (s, 1H), 7.72 (d, J = 8.8 Hz, 1H), | |
| | 7.57 (d, J = 9.6 Hz, 1H), 7.51 (d, J = 8.4 Hz, 1H), 7.02 (4t, J = 7.6 Hz, | |
| | 1H), 6.82 (d, J = 8.4 Hz, 1H), 4.51 (d, J = 13.2 Hz, 1H), 4.38 (s, 2H), | |
| | 4.00 (t, J = 6.0 Hz, 2H), 3.88 (t, J = 6.0 Hz, 2H), 3.79 (d, J = 12.4 Hz, | |
| | 1H), 3.23 (d, J = 8.4 Hz, 2H), 3.09 (t, J = 12.8 Hz, 1H), 3.00-2.90 (m, | |
| | 1H), 2.69-2.61 (m, 1H), 2.54 (s, 3H), 2.43-2.32 (m, 4H), 1.73 (s, 2H), | |
| | 1.52-1.33 (m, 2H) | |
| 456 | ¹H NMR (400 MHz, DMSO-d6) δ 10.95 (br s, 1H), 10.06-9.99 (m, | M |
| | 1H), 9.94 (s, 1H), 8.79 (s, 1H), 8.67 (d, J = 8.4 Hz, 1H), 8.44 (dd, J = | |
| | 6.0, 7.4 Hz, 1H), 7.87-7.77 (m, 2H), 7.71 (d, J = 8.5 Hz, 1H), 7.55- | |
| | 7.45 (m, 2H), 7.24 (d, J = 12.3 Hz, 1H), 7.14 (d, J = 8.6 Hz, 1H), 6.96 | |
| | (dt, J = 2.4, 7.6 Hz, 1H), 6.86 (d, J = 8.4 Hz, 1H), 4.38 (s, 2H), 3.82 (t, | |
| | J = 7.1 Hz, 1H), 3.11 (br d, J = 11.5 Hz, 3H), 2.80-2.72 (m, 2H), 2.69- | |
| | 2.64 (m, 2H), 2.58-2.56 (m, 1H), 2.35-2.30 (m, 1H), 2.21-2.10 (m, | |
| | 2H), 2.04-1.95 (m, 1H), 1.94-1.76 (m, 4H) | |
| 457 | ¹H NMR (400 MHz, DMSO-d6) δ 11.06 (s, 1H), 9.97 (s, 1H), 8.81 (s, | A |
| | 1H), 8.73 (d, J = 8.8 Hz, 1H), 8.49-8.40 (m, 1H), 7.82 (s, 1H), 7.71 (d, | |
| | J = 8.4 Hz, 1H), 7.65 (d, J = 8.4 Hz, 1H), 7.58-7.48 (m, 2H), 7.02- | |
| | 6.88 (m, 2H), 6.85-6.76 (m, 2H), 5.05 (dd, J = 5.2, 13.2 Hz, 1H), 4.38 | |
| | (s, 2H), 3.68-3.59 (m, 1H), 3.55-3.46 (m, 1H), 3.43-3.37 (m, 2H), | |
| | 3.12-3.01 (m, 3H), 2.94-2.84 (m, 1H), 2.73-2.55 (m, 3H), 2.46-2.29 | |
| | (m, 3H), 2.24-2.16 (m, 1H), 2.11-1.95 (m, 3H), 1.75-1.59 (m, 7H), | |
| | 1.35-1.16 (m, 2H) | |

Comparator “Compound A”, whose structure is shown below, was also synthesized according to known methods and tested in the biological assays disclosed below.

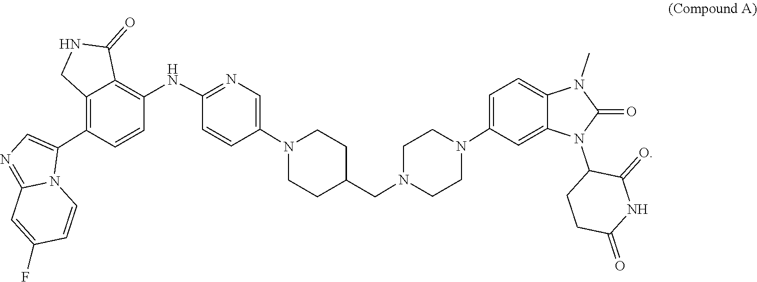 [SMILES: Cn1c(=O)n(C2CCC(=O)NC2=O)c2cc(N3CCN(CC4CCN(c5ccc(Nc6ccc(-c7cnc8cc(F)ccn78)c7c6C(=O)NC7)nc5)CC4)CC3)ccc21]

### Biological Assays

### Reagents

Jurkat cells were purchased from ATCC. Jurkat cells were cultured in GIBCO RPMI-1640 (ThermoFisher Scientific, catalog #61870-036, with Glutamax, no HEPES, no Na Pyruvate) supplemented with 10% FBS (ThermoFisher Scientific, catalog #2060357) and 1× Penicillin-Streptomycin (ThermoFisher Scientific, catalog #15140122, 10000 U/ml, 100×). For degradation assays, 384 well plates were used. Alamar blue was purchased from ThermoFisher Scientific (A50101) and used according to manufacturer instructions. The Nano-Glo® HiBiT Lytic Detection System and Nano-Glo® HiBiT Blotting System were purchased from Promega (#N3050 and #N2410) and used according to manufacturer instructions. MAP4K1 (HPK1) HiBit tagged Jurkat cell line was purchased from Promega Corporation and used as directed.

### HiBiT Degradation Assays

Jurkat HiBiT-HPK1 cells were plated at 10,000 cells/well in a 384 well plate or 50,000 cells/well in a 96 well plate. Compounds of the present disclosure and positive controls were diluted in the appropriate cell media and applied to the plated cells resulting in a final concentration titration of 1 μM to 17 pM in 0.5% DMSO. A reference compound was used as a positive control for cytotoxicity and HPK1 degradation and titrated from 1 μM to 17 pM. All assays were performed in triplicate. Cells were treated with compounds for 24 hr or 48 hr at 37° C. in an incubator containing 5% CO₂. Following treatment, alamar blue was used to determine if any compounds led to a loss in cell viability after 24 or 48 hr. Alamar blue reagent was added to assay plates at a 1:7 ratio. Assay Plates were returned to the incubator for 2 hrs and then stored at room temperature for 2 hrs. For alamar blue assay plates, fluorescence was measured using a Perkin-Elmer EnVision. HiBiT assay plates were allowed to equilibrate to room temperature. HiBiT detection reagent was prepared and added to each assay plate. Following incubation at room temperature for 45 min, the luminescence was read using the Perkin Elmer Envision instrument.

### Data Analysis

Fluorescence values from the alamar blue treatment were normalized to the DMSO-only control for each compound titration. HiBiT luminescence was normalized to the DMSO-only control and the fractional HiBiT signal was plotted versus the log of the PROTAC concentration and fit to a 4-parameter dose-response model to obtain the concentration of the compound that leads to half maximal degradation (DC₅₀) as well as the maximum degradation observed (Dₘₐₓ, conventionally expressed as a percentage of control).

HPK1 Target Engagement (pSLP76) Assay

Jurkat cells were plated at 200,000 cells/well in a 96 well plate (Corning #3628). Compounds of the present disclosure and positive controls were diluted in the appropriate cell media and applied to the plated cells resulting in a final concentration titration of 1 pM to 244 pM in 0.5% DMSO. A reference compound was used as a positive control for pSLP76 target engagement. All assays were performed in triplicate. Cells were treated with compounds for 24 hr at 37° C. in an incubator containing 5% CO₂. Following treatment, cells were transferred to a second 96 well plate that had been coated overnight at 4° C. with 5 ug/ml anti-CD3 antibody and soluble anti-CD28 was added to the culture at 1 ug/ml. Cells incubated on stimulation for 60 min at 37° C. in an incubator containing 5% CO₂. Cells were then collected, lysed, and assessed for pSLP76 levels following the manufacturer's protocol in the AlphaLISA SureFire Ultra p-SLP-76 (Ser376) Assay Kit. Total SLP76 was also assessed for normalization following AlphaLISA SureFire Ultra Total SLP-76 Assay Kit. Degradation data (Deg.) and pSLP78 Engagement Data is presented in the table below.

As can be seen, the compounds of the instant disclosure have significantly increased HPK1 degradation when compared to comparator Compound A.

| | | | Deg. | Deg. | Deg. | Deg. | Deg. | Deg. |
|---|---|---|---|---|---|---|---|---|
| | pSLP76 | pSLP76 | 48 hr | 48 hr | 48 hr | 48 hr | 24 hr | 24 hr |
| | EC₅₀ | Eₘₐₓ | DC₅₀ | Dₘₐₓ | DC₅₀ | Dₘₐₓ | DC₅₀ | Dmax |
| Ex. No. | (nM) | (%) | (nM) | (%) | (nM) | (%) | (nM) | (%) |
| 1 | B | A | A | B | B | B | B | B |
| 2 | B | A | NT | NT | NT | NT | A | B |
| 3 | A | A | B | A | B | A | C | B |
| 4 | A | A | NT | NT | NT | NT | B | B |
| 5 | C | A | D | A | NT | NT | NT | NT |
| 6 | A | A | A | A | NT | NT | NT | NT |
| 7 | B | A | B | A | NT | NT | NT | NT |
| 8 | A | A | B | A | NT | NT | NT | NT |
| 9 | B | A | B | B | B | A | A | A |
| 10 | B | B | A | B | NT | NT | NT | NT |
| 11 | B | A | A | C | NT | NT | NT | NT |
| 12 | B | A | A | A | NT | NT | NT | NT |
| 13 | B | A | A | A | B | A | B | A |
| 14 | D | C | D | NT | NT | NT | NT | NT |
| 15 | D | A | A | B | NT | NT | NT | NT |
| 16 | B | B | A | B | B | B | NT | NT |
| 17 | B | A | A | A | A | A | A | A |
| 18 | B | A | A | A | NT | NT | NT | NT |
| 19 | C | A | B | A | NT | NT | NT | NT |
| 20 | B | A | B | A | NT | NT | NT | NT |
| 21 | C | A | B | A | NT | NT | NT | NT |
| 22 | D | NT | D | NT | NT | NT | NT | NT |
| 23 | C | A | C | B | NT | NT | NT | NT |
| 24 | D | NT | D | NT | NT | NT | NT | NT |
| 25 | C | A | B | A | NT | NT | NT | NT |
| 26 | D | B | D | NT | A | B | B | C |
| 27 | D | A | D | B | C | A | NT | NT |
| 28 | C | B | B | C | NT | NT | NT | NT |
| 29 | B | A | A | B | NT | NT | NT | NT |
| 30 | B | A | B | B | NT | NT | NT | NT |
| 31 | C | A | B | B | NT | NT | NT | NT |
| 32 | C | A | C | A | NT | NT | NT | NT |
| 33 | B | A | A | B | A | A | A | B |
| 34 | B | A | B | B | NT | NT | NT | NT |
| 35 | B | A | B | B | NT | NT | NT | NT |
| 36 | B | A | A | A | NT | NT | NT | NT |
| 37 | B | B | B | B | B | A | C | B |
| 38 | B | A | A | A | A | A | A | B |
| 39 | B | A | B | A | C | A | B | A |
| 40 | C | A | A | B | NT | NT | NT | NT |
| 41 | B | A | C | A | A | A | A | A |
| 42 | C | B | C | B | NT | NT | NT | NT |
| 43 | B | A | B | A | NT | NT | NT | NT |
| 44 | A | A | A | A | A | A | A | A |
| 45 | B | A | B | B | B | A | A | B |
| 46 | B | A | A | A | B | A | B | B |
| 47 | B | A | B | A | A | A | A | A |
| 48 | B | A | B | B | A | A | A | B |
| 49 | B | A | C | A | A | A | A | B |
| 50 | C | A | C | A | NT | NT | NT | NT |
| 51 | C | A | C | A | A | A | A | A |
| 52 | B | A | B | B | A | B | A | B |
| 53 | B | A | C | A | NT | NT | NT | NT |
| 54 | B | A | B | A | NT | NT | NT | NT |
| 55 | C | A | C | A | NT | NT | NT | NT |
| 56 | B | A | A | A | NT | NT | NT | NT |
| 57 | B | A | B | A | NT | NT | NT | NT |
| 58 | B | A | B | A | NT | NT | NT | NT |
| 59 | D | A | B | B | NT | NT | NT | NT |
| 60 | B | A | B | A | NT | NT | NT | NT |
| 61 | C | A | C | B | NT | NT | NT | NT |
| 62 | C | A | C | A | NT | NT | NT | NT |
| 63 | C | A | C | A | NT | NT | NT | NT |
| 64 | C | A | C | A | B | A | NT | NT |
| 65 | B | A | B | A | NT | NT | NT | NT |
| 66 | C | A | B | B | B | A | B | B |
| 67 | C | A | B | A | A | A | B | A |
| 68 | C | A | B | B | NT | NT | NT | NT |
| 69 | B | A | B | A | A | A | A | A |
| 70 | C | A | C | A | NT | NT | NT | NT |
| 71 | B | A | C | A | B | A | B | A |
| 72 | C | A | C | A | B | A | C | A |
| 73 | B | A | D | NT | B | A | B | A |
| 74 | B | A | A | A | B | B | A | B |
| 75 | B | A | C | A | B | A | B | B |
| 76 | B | A | B | B | A | A | A | B |
| 77 | B | A | B | B | NT | NT | NT | NT |
| 78 | B | A | B | A | NT | NT | NT | NT |
| 79 | B | A | B | A | NT | NT | NT | NT |
| 80 | B | A | B | A | A | A | A | A |
| 81 | B | A | C | B | B | A | B | B |
| 82 | B | A | B | A | NT | NT | NT | NT |
| 83 | A | A | A | B | NT | NT | NT | NT |
| 84 | D | NT | D | NT | NT | NT | NT | NT |
| 85 | B | A | B | B | B | A | B | B |
| 86 | A | A | NT | NT | NT | NT | NT | NT |
| 87 | B | A | B | A | B | A | B | A |
| 88 | B | A | B | A | NT | NT | NT | NT |
| 89 | B | A | A | A | NT | NT | NT | NT |
| 90 | B | A | A | A | NT | NT | NT | NT |
| 91 | B | A | B | B | NT | NT | NT | NT |
| 92 | B | A | B | B | NT | NT | NT | NT |
| 93 | C | A | C | A | NT | NT | NT | NT |
| 94 | C | A | B | B | B | B | A | B |
| 95 | B | A | A | B | A | A | A | B |
| 96 | A | A | B | A | B | A | NT | NT |
| 97 | A | A | A | A | C | A | B | B |
| 98 | A | A | A | B | NT | NT | NT | NT |
| 99 | B | A | C | B | B | B | B | B |
| 100 | A | A | B | A | B | A | NT | NT |
| 101 | C | A | A | C | B | B | A | B |
| 102 | B | A | B | C | B | B | B | B |
| 103 | C | A | B | A | B | A | C | A |
| 104 | B | A | B | A | B | A | B | A |
| 105 | B | A | A | A | NT | NT | NT | NT |
| 106 | B | A | C | A | C | A | B | A |
| 107 | A | A | B | A | NT | NT | NT | NT |
| 108 | C | A | C | C | B | B | B | B |
| 109 | A | A | B | B | B | A | C | B |
| 110 | B | A | A | A | A | A | A | A |
| 111 | B | A | B | B | B | A | B | B |
| 112 | B | A | NT | NT | B | A | B | B |
| 113 | B | A | NT | NT | B | A | B | A |
| 114 | B | A | NT | NT | B | A | NT | NT |
| 115 | D | A | NT | NT | C | B | C | B |
| 116 | C | A | NT | NT | B | B | C | B |
| 117 | C | A | NT | NT | B | B | C | B |
| 118 | C | A | NT | NT | C | A | NT | NT |
| 119 | A | A | NT | NT | B | A | B | A |
| 120 | B | A | NT | NT | B | A | B | B |
| 121 | A | A | NT | NT | B | A | B | B |
| 122 | A | A | NT | NT | B | A | NT | NT |
| 123 | D | A | NT | NT | B | A | B | A |
| 124 | B | A | NT | NT | B | A | NT | NT |
| 125 | B | A | NT | NT | B | A | B | A |
| 126 | B | A | NT | NT | B | A | B | B |
| 127 | B | A | NT | NT | A | A | NT | NT |
| 128 | C | A | NT | NT | B | A | B | A |
| 129 | NT | NT | NT | NT | B | A | B | B |
| 130 | C | B | NT | NT | C | B | C | B |
| 131 | D | B | NT | NT | D | A | NT | NT |
| 132 | B | A | NT | NT | A | A | B | B |
| 133 | B | A | NT | NT | B | B | B | B |
| 134 | B | A | NT | NT | B | B | NT | NT |
| 135 | NT | NT | NT | NT | B | B | C | B |
| 136 | C | A | NT | NT | A | A | B | A |
| 137 | NT | NT | NT | NT | B | A | B | A |
| 138 | C | A | NT | NT | A | B | B | B |
| 139 | NT | NT | NT | NT | A | B | NT | NT |
| 140 | NT | NT | NT | NT | B | B | NT | NT |
| 141 | NT | NT | NT | NT | B | A | B | B |
| 142 | C | A | NT | NT | B | B | NT | NT |
| 143 | C | A | NT | NT | B | B | NT | NT |
| 144 | NT | NT | NT | NT | A | A | NT | NT |
| 145 | NT | NT | NT | NT | A | A | NT | NT |
| 146 | NT | NT | NT | NT | A | A | B | A |
| 147 | NT | NT | NT | NT | B | A | NT | NT |
| 148 | NT | NT | NT | NT | A | B | A | B |
| 149 | NT | NT | NT | NT | C | A | NT | NT |
| 150 | NT | NT | NT | NT | A | A | B | B |
| 151 | NT | NT | NT | NT | D | NT | NT | NT |
| 152 | NT | NT | NT | NT | C | B | C | B |
| 153 | D | NT | NT | NT | B | B | NT | NT |
| 154 | NT | NT | NT | NT | B | A | B | A |
| 155 | B | A | NT | NT | B | A | B | B |
| 156 | NT | NT | NT | NT | B | A | NT | NT |
| 157 | D | A | NT | NT | NT | NT | NT | NT |
| 158 | NT | NT | NT | NT | B | A | B | A |
| 159 | NT | NT | NT | NT | B | B | B | B |
| 160 | B | A | NT | NT | A | A | B | A |
| 161 | B | A | NT | NT | B | A | NT | NT |
| 162 | B | A | NT | NT | B | A | NT | NT |
| 163 | D | A | NT | NT | C | B | NT | NT |
| 164 | NT | NT | NT | NT | A | B | A | B |
| 165 | NT | NT | NT | NT | C | A | B | A |
| 166 | NT | NT | NT | NT | B | A | B | B |
| 167 | B | A | NT | NT | B | A | B | A |
| 168 | NT | NT | NT | NT | B | A | B | B |
| 169 | NT | NT | NT | NT | B | A | NT | NT |
| 170 | NT | NT | NT | NT | C | A | NT | NT |
| 171 | NT | NT | NT | NT | B | A | B | B |
| 172 | NT | NT | NT | NT | B | A | NT | NT |
| 173 | NT | NT | NT | NT | C | A | NT | NT |
| 174 | NT | NT | NT | NT | C | A | C | A |
| 175 | NT | NT | NT | NT | B | A | B | A |
| 176 | NT | NT | NT | NT | C | B | C | B |
| 177 | NT | NT | NT | NT | A | A | NT | NT |
| 178 | NT | NT | NT | NT | C | A | C | B |
| 179 | B | A | NT | NT | B | A | B | A |
| 180 | NT | NT | NT | NT | C | A | C | A |
| 181 | NT | NT | NT | NT | C | B | C | B |
| 182 | B | A | NT | NT | NT | NT | NT | NT |
| 183 | NT | NT | NT | NT | B | A | C | B |
| 184 | NT | NT | NT | NT | NT | NT | NT | NT |
| 185 | NT | NT | NT | NT | B | A | B | A |
| 186 | NT | NT | NT | NT | C | B | NT | NT |
| 187 | NT | NT | NT | NT | B | A | A | A |
| 188 | NT | NT | NT | NT | B | A | A | B |
| 189 | NT | NT | NT | NT | B | A | B | B |
| 190 | NT | NT | NT | NT | C | A | B | A |
| 191 | NT | NT | NT | NT | NT | NT | NT | NT |
| 192 | NT | NT | NT | NT | C | C | C | B |
| 193 | NT | NT | NT | NT | NT | NT | NT | NT |
| 194 | NT | NT | NT | NT | A | A | A | A |
| 195 | NT | NT | NT | NT | C | A | B | A |
| 196 | NT | NT | NT | NT | C | A | C | A |
| 197 | B | A | NT | NT | C | A | B | A |
| 198 | NT | NT | NT | NT | D | B | D | B |
| 199 | NT | NT | NT | NT | NT | NT | B | A |
| 200 | B | A | NT | NT | NT | NT | B | A |
| 201 | B | A | NT | NT | NT | NT | B | A |
| 202 | NT | NT | NT | NT | NT | NT | B | A |
| 203 | NT | NT | NT | NT | NT | NT | B | A |
| 204 | NT | NT | NT | NT | NT | NT | C | B |
| 205 | NT | NT | NT | NT | NT | NT | A | A |
| 206 | A | A | NT | NT | NT | NT | B | B |
| 207 | C | A | NT | NT | NT | NT | C | B |
| 208 | C | A | NT | NT | NT | NT | C | B |
| 209 | A | A | NT | NT | NT | NT | C | B |
| 210 | A | A | NT | NT | NT | NT | B | A |
| 211 | NT | NT | NT | NT | NT | NT | C | B |
| 212 | NT | NT | NT | NT | NT | NT | B | B |
| 213 | NT | NT | NT | NT | NT | NT | NT | NT |
| 214 | B | A | NT | NT | NT | NT | A | B |
| 215 | NT | NT | NT | NT | NT | NT | B | B |
| 216 | NT | NT | NT | NT | NT | NT | NT | NT |
| 217 | NT | NT | NT | NT | NT | NT | B | A |
| 218 | NT | NT | NT | NT | NT | NT | D | B |
| 219 | A | A | NT | NT | NT | NT | A | A |
| 220 | A | A | NT | NT | NT | NT | B | A |
| 221 | NT | NT | NT | NT | NT | NT | B | A |
| 222 | NT | NT | NT | NT | NT | NT | B | A |
| 223 | NT | NT | NT | NT | NT | NT | B | A |
| 224 | B | A | NT | NT | NT | NT | A | B |
| 225 | B | A | NT | NT | NT | NT | A | B |
| 226 | C | A | NT | NT | NT | NT | C | B |
| 227 | NT | NT | NT | NT | NT | NT | A | B |
| 228 | A | A | NT | NT | NT | NT | B | B |
| 229 | NT | NT | NT | NT | NT | NT | B | A |
| 230 | NT | NT | NT | NT | NT | NT | C | B |
| 231 | NT | NT | NT | NT | NT | NT | D | C |
| 232 | A | A | NT | NT | NT | NT | B | B |
| 233 | NT | NT | NT | NT | NT | NT | NT | NT |
| 234 | NT | NT | NT | NT | NT | NT | B | A |
| 235 | NT | NT | NT | NT | NT | NT | C | B |
| 236 | NT | NT | NT | NT | NT | NT | C | B |
| 237 | A | A | NT | NT | NT | NT | NT | NT |
| 238 | NT | NT | NT | NT | NT | NT | B | A |
| 239 | NT | NT | NT | NT | NT | NT | B | A |
| 240 | B | A | NT | NT | NT | NT | NT | NT |
| 241 | B | A | NT | NT | NT | NT | A | A |
| 242 | D | A | NT | NT | NT | NT | D | B |
| 243 | NT | NT | NT | NT | NT | NT | B | B |
| 244 | NT | NT | NT | NT | NT | NT | B | B |
| 245 | B | A | NT | NT | NT | NT | B | B |
| 246 | NT | NT | NT | NT | NT | NT | NT | NT |
| 247 | A | A | NT | NT | NT | NT | B | B |
| 248 | NT | NT | NT | NT | NT | NT | D | C |
| 249 | D | A | NT | NT | NT | NT | C | C |
| 250 | NT | NT | NT | NT | NT | NT | C | B |
| 251 | NT | NT | NT | NT | NT | NT | B | A |
| 252 | C | A | NT | NT | NT | NT | C | B |
| 253 | NT | NT | NT | NT | NT | NT | NT | NT |
| 254 | A | A | NT | NT | NT | NT | A | B |
| 255 | A | A | NT | NT | NT | NT | A | A |
| 256 | D | NT | NT | NT | NT | NT | D | NT |
| 257 | D | A | NT | NT | NT | NT | C | C |
| 258 | A | A | NT | NT | NT | NT | C | B |
| 259 | B | B | NT | NT | NT | NT | A | B |
| 260 | A | A | NT | NT | NT | NT | A | B |
| 261 | D | A | NT | NT | NT | NT | C | B |
| 262 | B | A | NT | NT | NT | NT | B | B |
| 263 | NT | NT | NT | NT | NT | NT | D | B |
| 264 | D | A | NT | NT | NT | NT | D | C |
| 265 | D | B | NT | NT | NT | NT | D | C |
| 266 | NT | NT | NT | NT | NT | NT | B | B |
| 267 | B | A | NT | NT | NT | NT | B | A |
| 268 | C | A | NT | NT | NT | NT | B | B |
| 269 | C | A | NT | NT | NT | NT | C | B |
| 270 | A | A | NT | NT | NT | NT | A | B |
| 271 | A | A | NT | NT | NT | NT | A | A |
| 272 | NT | NT | NT | NT | NT | NT | D | NT |
| 273 | NT | NT | NT | NT | NT | NT | D | A |
| 274 | B | A | NT | NT | NT | NT | B | B |
| 275 | B | A | NT | NT | NT | NT | B | B |
| 276 | B | A | NT | NT | NT | NT | A | B |
| 277 | B | A | NT | NT | NT | NT | B | A |
| 278 | NT | NT | NT | NT | NT | NT | C | A |
| 279 | NT | NT | NT | NT | NT | NT | C | A |
| 280 | A | A | NT | NT | NT | NT | A | B |
| 281 | A | A | NT | NT | NT | NT | A | B |
| 282 | A | A | NT | NT | NT | NT | A | A |
| 283 | B | A | NT | NT | NT | NT | A | B |
| 284 | A | A | NT | NT | NT | NT | A | A |
| 285 | B | A | NT | NT | NT | NT | A | B |
| 286 | B | A | NT | NT | NT | NT | A | A |
| 287 | B | A | NT | NT | NT | NT | B | A |
| 288 | NT | NT | NT | NT | NT | NT | B | A |
| 289 | NT | NT | NT | NT | NT | NT | C | B |
| 290 | B | A | NT | NT | NT | NT | A | A |
| 291 | D | C | NT | NT | NT | NT | NT | NT |
| 292 | D | NT | NT | NT | NT | NT | D | NT |
| 293 | B | A | NT | NT | NT | NT | A | B |
| 294 | C | A | NT | NT | NT | NT | C | B |
| 295 | D | A | NT | NT | NT | NT | D | C |
| 296 | B | A | NT | NT | A | A | B | B |
| 297 | B | A | NT | NT | B | A | B | A |
| 298 | NT | NT | NT | NT | NT | NT | C | B |
| 299 | NT | NT | NT | NT | NT | NT | D | B |
| 300 | D | C | NT | NT | NT | NT | NT | NT |
| 301 | C | A | C | A | NT | NT | NT | NT |
| 302 | C | A | NT | NT | NT | NT | C | B |
| 303 | NT | NT | NT | NT | NT | NT | D | C |
| 304 | NT | NT | NT | NT | NT | NT | NT | NT |
| 305 | NT | NT | NT | NT | D | NT | D | NT |
| 306 | NT | NT | NT | NT | D | NT | D | NT |
| 307 | NT | NT | NT | NT | C | C | D | NT |
| 308 | NT | NT | NT | NT | D | NT | D | NT |
| 309 | NT | NT | NT | NT | D | NT | D | C |
| 310 | B | A | NT | NT | NT | NT | B | A |
| 311 | NT | NT | NT | NT | NT | NT | NT | NT |
| 312 | NT | NT | NT | NT | NT | NT | NT | NT |
| 313 | NT | NT | NT | NT | B | B | B | B |
| 314 | NT | NT | NT | NT | B | B | C | C |
| 315 | NT | NT | NT | NT | D | C | D | C |
| 316 | NT | NT | NT | NT | B | B | B | B |
| 317 | NT | NT | NT | NT | B | B | B | B |
| 318 | NT | NT | NT | NT | D | NT | D | NT |
| 319 | NT | NT | NT | NT | C | C | C | C |
| 320 | NT | NT | NT | NT | NT | NT | A | B |
| 321 | NT | NT | NT | NT | NT | NT | C | B |
| 322 | NT | NT | NT | NT | B | B | B | C |
| 323 | NT | NT | NT | NT | NT | NT | C | B |
| 324 | NT | NT | NT | NT | A | B | A | B |
| 325 | NT | NT | NT | NT | NT | NT | C | C |
| 326 | C | A | NT | NT | NT | NT | C | C |
| 327 | NT | NT | NT | NT | NT | NT | D | NT |
| 328 | D | A | NT | NT | NT | NT | D | C |
| 329 | NT | NT | NT | NT | NT | NT | C | C |
| 330 | NT | NT | NT | NT | NT | NT | C | B |
| 331 | NT | NT | NT | NT | NT | NT | A | B |
| 332 | B | A | NT | NT | NT | NT | C | B |
| 333 | NT | NT | NT | NT | NT | NT | C | B |
| 334 | NT | NT | NT | NT | NT | NT | C | B |
| 335 | NT | NT | NT | NT | NT | NT | D | B |
| 336 | NT | NT | NT | NT | NT | NT | B | B |
| 337 | NT | NT | NT | NT | NT | NT | C | B |
| 338 | NT | NT | NT | NT | NT | NT | C | B |
| 339 | NT | NT | NT | NT | NT | NT | C | C |
| 340 | NT | NT | NT | NT | NT | NT | C | B |
| 341 | NT | NT | NT | NT | NT | NT | C | B |
| 342 | NT | NT | NT | NT | NT | NT | C | B |
| 343 | NT | NT | NT | NT | NT | NT | D | B |
| 344 | NT | NT | NT | NT | NT | NT | C | C |
| 345 | B | A | NT | NT | NT | NT | C | A |
| 346 | B | A | NT | NT | NT | NT | C | A |
| 347 | NT | NT | NT | NT | NT | NT | C | A |
| 348 | B | A | NT | NT | NT | NT | B | B |
| 349 | B | A | NT | NT | NT | NT | C | A |
| 350 | B | A | NT | NT | NT | NT | A | B |
| 351 | C | A | NT | NT | NT | NT | A | B |
| 352 | C | A | NT | NT | NT | NT | C | B |
| 353 | C | A | NT | NT | NT | NT | C | B |
| 354 | C | A | NT | NT | NT | NT | C | A |
| 355 | C | A | NT | NT | NT | NT | C | B |
| 356 | C | A | NT | NT | NT | NT | C | B |
| 357 | C | A | NT | NT | NT | NT | NT | NT |
| 358 | B | A | NT | NT | NT | NT | B | A |
| 359 | A | A | NT | NT | NT | NT | A | B |
| 360 | A | A | NT | NT | NT | NT | B | B |
| 361 | B | A | NT | NT | NT | NT | C | A |
| 362 | B | A | NT | NT | NT | NT | C | B |
| 363 | D | A | NT | NT | NT | NT | C | C |
| 364 | B | A | NT | NT | NT | NT | B | B |
| 365 | D | B | NT | NT | NT | NT | C | C |
| 366 | B | A | NT | NT | NT | NT | B | B |
| 367 | NT | NT | NT | NT | NT | NT | A | B |
| 368 | C | A | NT | NT | NT | NT | C | A |
| 369 | NT | NT | NT | NT | NT | NT | D | B |
| 370 | C | A | NT | NT | NT | NT | B | B |
| 371 | A | A | NT | NT | NT | NT | B | B |
| 372 | D | A | NT | NT | NT | NT | D | B |
| 373 | A | A | NT | NT | NT | NT | B | A |
| 374 | NT | NT | NT | NT | NT | NT | B | A |
| 375 | B | A | NT | NT | NT | NT | B | A |
| 376 | C | A | NT | NT | NT | NT | C | A |
| 377 | NT | NT | NT | NT | B | B | B | B |
| 378 | NT | NT | NT | NT | B | A | B | A |
| 379 | NT | NT | NT | NT | B | A | B | B |
| 380 | NT | NT | NT | NT | B | A | C | A |
| 381 | B | A | C | A | NT | NT | NT | NT |
| 382 | B | A | B | B | A | A | A | A |
| 383 | D | A | D | A | NT | NT | NT | NT |
| 384 | B | A | NT | NT | NT | NT | B | A |
| 385 | A | A | NT | NT | NT | NT | A | B |
| 386 | A | A | NT | NT | NT | NT | B | B |
| 387 | NT | NT | NT | NT | B | A | B | B |
| 388 | B | A | A | A | NT | NT | NT | NT |
| 389 | B | A | A | A | B | A | B | A |
| 390 | NT | NT | NT | NT | A | B | A | C |
| 391 | NT | NT | NT | NT | B | B | B | B |
| 392 | B | A | NT | NT | NT | NT | B | B |
| 393 | A | A | NT | NT | NT | NT | A | B |
| 394 | NT | NT | NT | NT | NT | NT | A | B |
| 395 | C | A | NT | NT | B | B | B | B |
| 396 | NT | NT | NT | NT | NT | NT | B | B |
| 397 | B | A | NT | NT | NT | NT | A | B |
| 398 | B | A | NT | NT | NT | NT | A | B |
| 399 | B | A | NT | NT | NT | NT | B | B |
| 400 | C | A | NT | NT | NT | NT | C | B |
| 401 | NT | NT | NT | NT | NT | NT | B | B |
| 402 | A | A | NT | NT | NT | NT | B | A |
| 403 | NT | NT | NT | NT | NT | NT | C | B |
| 404 | A | A | NT | NT | NT | NT | A | B |
| 405 | NT | NT | NT | NT | B | A | B | A |
| 406 | B | A | NT | NT | NT | NT | NT | NT |
| 407 | NT | NT | NT | NT | NT | NT | B | A |
| 408 | C | A | NT | NT | NT | NT | C | A |
| 409 | A | A | NT | NT | NT | NT | B | A |
| 410 | NT | NT | NT | NT | NT | NT | B | A |
| 411 | B | A | NT | NT | NT | NT | B | B |
| 412 | NT | NT | NT | NT | NT | NT | B | B |
| 413 | NT | NT | NT | NT | NT | NT | A | B |
| 414 | NT | NT | NT | NT | NT | NT | B | B |
| 415 | B | A | NT | NT | NT | NT | B | A |
| 416 | NT | NT | NT | NT | C | B | C | B |
| 417 | NT | NT | NT | NT | C | B | C | B |
| 418 | NT | NT | NT | NT | B | A | B | A |
| 419 | C | A | NT | NT | B | A | B | B |
| 420 | NT | NT | NT | NT | B | A | B | A |
| 421 | NT | NT | NT | NT | C | A | C | B |
| 422 | B | A | C | A | B | A | C | A |
| 423 | C | A | C | B | NT | NT | NT | NT |
| 424 | C | A | C | A | NT | NT | NT | NT |
| 425 | C | A | C | A | NT | NT | NT | NT |
| 426 | D | NT | D | NT | NT | NT | NT | NT |
| 427 | C | A | C | A | NT | NT | NT | NT |
| 428 | B | A | NT | NT | NT | NT | B | A |
| 429 | NT | NT | NT | NT | NT | NT | B | B |
| 430 | B | A | NT | NT | NT | NT | B | A |
| 431 | B | A | NT | NT | NT | NT | A | B |
| 432 | C | A | C | A | NT | NT | NT | NT |
| 433 | A | A | NT | NT | NT | NT | A | B |
| 434 | B | A | C | A | B | A | B | A |
| 435 | C | A | NT | NT | B | A | B | A |
| 436 | A | A | NT | NT | C | B | C | B |
| 437 | D | A | NT | NT | A | B | B | B |
| 438 | B | A | NT | NT | NT | NT | B | B |
| 439 | NT | NT | NT | NT | NT | NT | C | B |
| 440 | NT | NT | NT | NT | NT | NT | D | B |
| 441 | D | A | NT | NT | NT | NT | NT | NT |
| 442 | NT | NT | NT | NT | NT | NT | D | B |
| 443 | NT | NT | NT | NT | NT | NT | D | C |
| 444 | C | A | NT | NT | C | B | B | B |
| 445 | B | C | NT | NT | NT | NT | C | C |
| 446 | NT | NT | D | NT | NT | NT | NT | NT |
| 447 | C | A | C | A | NT | NT | NT | NT |
| 448 | D | A | C | B | NT | NT | NT | NT |
| 449 | D | NT | C | B | B | B | NT | NT |
| 450 | D | A | D | NT | D | A | NT | NT |
| 451 | C | B | C | B | NT | NT | NT | NT |
| 452 | D | NT | D | B | NT | NT | NT | NT |
| 453 | NT | NT | NT | NT | B | B | B | B |
| 454 | NT | NT | NT | NT | B | A | B | A |
| 455 | NT | NT | NT | NT | A | B | B | B |
| 456 | NT | NT | NT | NT | NT | NT | D | NT |
| 457 | B | A | B | B | C | A | C | A |
| Cmp | C | A | NT | NT | D | D | D | D |
| A | | | | | | | | |

For pSLP76 data:
A = Eₘₐₓ ≥ 75%; B = Eₘₐₓ < 75% and Eₘₐₓ ≥ 50%; C = Eₘₐₓ < 50% and A = EC₅₀ < 5 nM; B = EC₅₀ ≥ 5 nM and EC₅₀ < 20 nM; C = EC₅₀ ≥ 20 nM and EC₅₀ < 100 nM; D = EC₅₀ ≥ 100 nM
For Degradation data:
A = Dₘₐₓ ≥ 75%; B = Dₘₐₓ < 75% and Dₘₐₓ ≥ 50%; C = Dₘₐₓ < 50% and A = DC₅₀ <5 nM; B = DC₅₀ ≥ 5 nM and DC₅₀ < 20 nM; C = DC₅₀ ≥ 20 nM and DC₅₀ < 100 nM; D = DC₅₀ ≥ 100 nM
NT = Not Tested

### PK Assays

Single dose IV PK experiments, dosed at 1 mg/kg, were conducted using a formulation composed of either (a) 10% HP-β-CD (w/v) in water containing 40 mM sodium acetate and 40 mM NaCl (pH 4.5) or (b) 30% SBE-β-CD in 10 mM citrate (pH 3.0). Single dose PO (oral gavage) PK experiments, administered at 10 mg/kg, were conducted using a formulation composed of either (a) 30% SBE-P-CD in 10 mM citrate (pH 3.0), (b) 10% DMSO and 90% [2% Tween80/PEG400], or (c) 20% HP-β-CD in 10 mM citrate buffer (pH>3.0). Male CD-1 mice, female Balb/c mice, or male Wistar Han rats were fasted overnight prior to dosing and food was returned 4 hours after dosing. For PO dosed studies, water was removed from one hour prior to dosing through one hour post compound administration. Otherwise, water was available ad libitum for all studies. IV samples were collected at timepoints 0.033, 0.083, 0.25, 0.5, 1, 2, 4, 8 and 24 hours post IV administration; PO samples were collected at timepoints 0.25, 0.5, 1, 2, 4, 8 and 24 hours post oral gavage. Approximately 0.2 mL blood was collected at each time point from the from the jugular vein, then put on wet ice, centrifuged at 2000 g for 5 minutes at 4° C. within 30 minutes of collection. Concentrations of compound in the plasma samples were analyzed using LC-MS/MS method and using an internal standard. Clearance (IV) and F % (PO) were calculated by WinNonlin (Phoenix™, version 6.1 or later).

As shown in the tables below, in some instances it was discovered an isopropyl or cylcopropyl at the R^{E1} position and, optionally, a methyl at the R³ position led to compounds with significantly higher oral bioavailability.

| | Winstar Han | pSLP76 | pSLP76 | Deg. 24 hr | Deg. 24 hr |
|---|---|---|---|---|---|
| Ex. | Rat | EC₅₀ | Eₘₐₓ | 384 well DC₅₀ | 384 well Dₘₐₓ |
| No. | PO F (%) | (nM) | (%) | (nM) | (%) |
| 1 | B | B | A | B | B |
| 4 | D | B | A | B | B |
| 45 | C | B | A | B | B |
| 73 | B | B | A | B | B |
| 94 | D | C | A | B | B |
| 134 | D | B | A | B | B |
| 182 | C | B | A | NT | NT |
| 228 | D | A | A | B | B |
| 245 | C | B | A | B | B |
| 247 | C | C | A | B | B |
| 280 | D | A | A | A | B |
| 393 | C | C | A | C | B |
| 395 | A | C | A | B | B |

For pSLP76 data: A = Eₘₐₓ ≥75%; B = Eₘₐₓ <75% and Eₘₐₓ ≥50%; C = Eₘₐₓ <50% and A = EC₅₀ <5 nM; B = EC₅₀ ≥5 nM and EC₅₀ <20 nM; C = EC₅₀ ≥20 nM and EC₅₀ <100 nM; D = EC₅₀ ≥100 nM
For Degradation data: A = Dₘₐₓ ≥75%; B = Dₘₐₓ <75% and Dₘₐₓ ≥50%; C = Dₘₐₓ <50% and A = DC₅₀ <5 nM; B = DC₅₀ ≥5 nM and DC₅₀ <20 nM; C = DC₅₀ ≥20 nM and DC₅₀ <100 nM; D = DC₅₀ ≥100 nM
For PO F (%): A = ≥75%; B = <75% and ≥50%; C = <50% and ≥25%; D = <25%
NT = Not Tested

### In-Vivo Assay

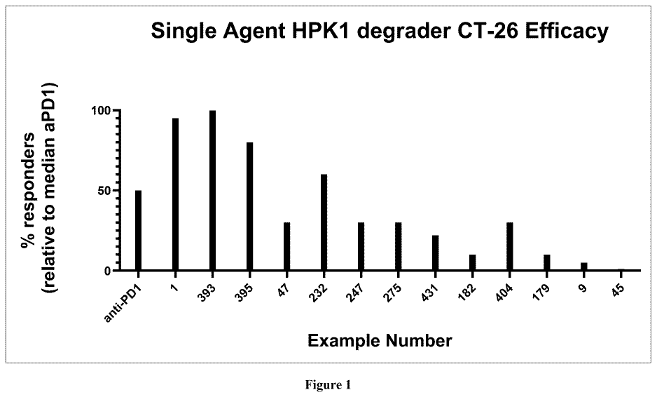

A CT-26 murine syngeneic tumor cell line was implanted subcutaneously into the flank of female BALB/c mice. After allowing the tumors to establish and grow to measurable size, mice were randomized into different arms (n=10 mice per arm) so that average tumor volume was consistent across groups. Mice were then treated with either vehicle or 30-60 mg/kg QD of HPK1 PROTAC compound via PO route of administration. As a benchmark control for each experiment, a 10 mg/kg dose of anti-PD1 antibody via IP injection was administered to one of the arms. Tumors were measured twice a week and the number of “responding tumors” (i.e. tumor volume less than the median of anti-PD1 Ab response) was assessed at the end of the study interval (14-21 days of treatment). The result of this study is shown in FIG. 1.

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

What is claimed is:

1. A compound of Formula (V): 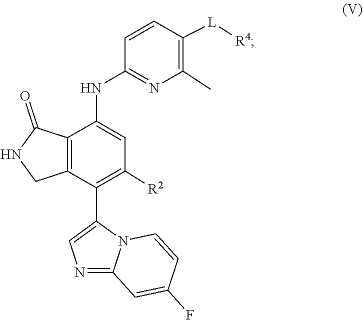
or a pharmaceutically acceptable salt thereof, wherein:
R² is H or C₁₋₄alkyl;
L is 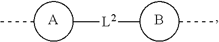
wherein:
Rings A and B are each independently 6-membered monocyclic heterocyclyl;
L² is absent or CH₂;
R⁴ is 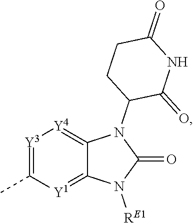
wherein:
R^{E1} is C₁₋₃alkyl or C₃₋₄cycloalkyl;
Y¹ is N or CR^{Y};
Y³ and Y⁴ are each independently CR^{Y};
each R^{Y} is independently hydrogen or halo.

2. The compound of claim 1, wherein R² is H or CH₃.

3. The compound of claim 1, wherein the compound is of Formula (III): 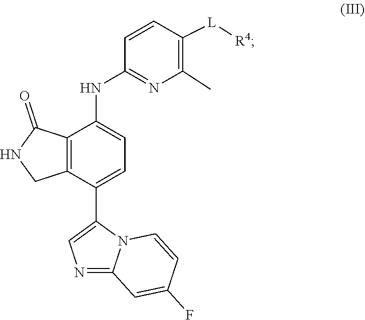
or a pharmaceutically acceptable salt thereof, wherein:
L is 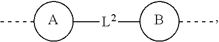
wherein:
Rings A and B are each independently 6-membered monocyclic heterocyclyl;
L² is absent or CH₂;
R⁴ is 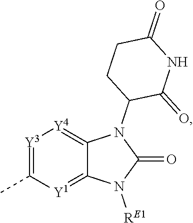
wherein:
R^{E1} is C₁₋₃alkyl or C₃₋₄cycloalkyl;
Y¹ is N or CR^{Y};
Y³ and Y⁴ are each independently CR^{Y};
each R^{Y} is independently hydrogen or halo.

4. The compound of claim 1, or a pharmaceutically acceptable salt thereof, wherein Rings A and B are each independently piperazinyl or piperidinyl.

5. The compound of claim 4, or a pharmaceutically acceptable salt thereof, wherein Ring A is piperazinyl and Ring B is piperdinyl.

6. The compound of claim 1, or a pharmaceutically acceptable salt thereof, wherein L² is —CH₂—.

7. The compound of claim 1, or a pharmaceutically acceptable salt thereof, wherein L² is absent.

8. The compound of claim 1, or a pharmaceutically acceptable salt thereof, wherein R⁴ is

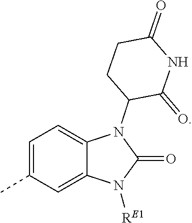

9. The compound claim 8, or a pharmaceutically acceptable salt thereof, wherein R^{E1} is —CH(CH₃)₂.

10. The compound of claim 8, or a pharmaceutically acceptable salt thereof, wherein R^{E1} is cyclopropyl.

11. The compound of claim 3, wherein the compound is: 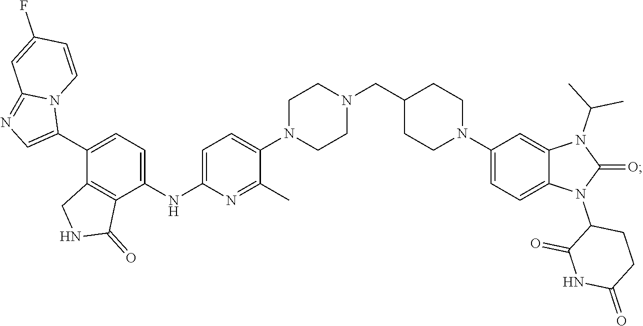 [SMILES: Cc1nc(Nc2ccc(-c3cnc4cc(F)ccn34)c3c2C(=O)NC3)ccc1N1CCN(CC2CCN(c3ccc4c(c3)n(C(C)C)c(=O)n4C3CCC(=O)NC3=O)CC2)CC1]
or a pharmaceutically acceptable salt thereof.

12. The compound of claim 3, wherein the compound is: 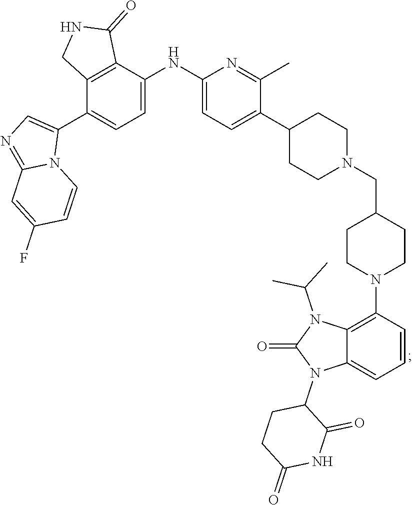 [SMILES: Cc1nc(Nc2ccc(-c3cnc4cc(F)ccn34)c3c2C(=O)NC3)ccc1C1CCN(CC2CCN(c3cccc4c3n(C(C)C)c(=O)n4C3CCC(=O)NC3=O)CC2)CC1]
or a pharmaceutically acceptable salt thereof.

13. The compound of claim 3, wherein the compound is: 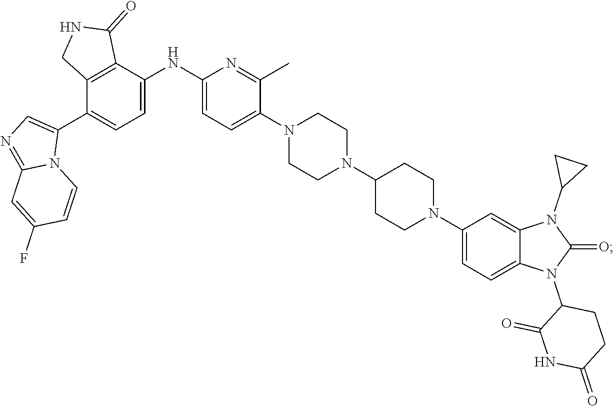 [SMILES: Cc1nc(Nc2ccc(-c3cnc4cc(F)ccn34)c3c2C(=O)NC3)ccc1N1CCN(C2CCN(c3ccc4c(c3)n(C3CC3)c(=O)n4C3CCC(=O)NC3=O)CC2)CC1]
or a pharmaceutically acceptable salt thereof.

14. The compound of claim 3, wherein the compound is: 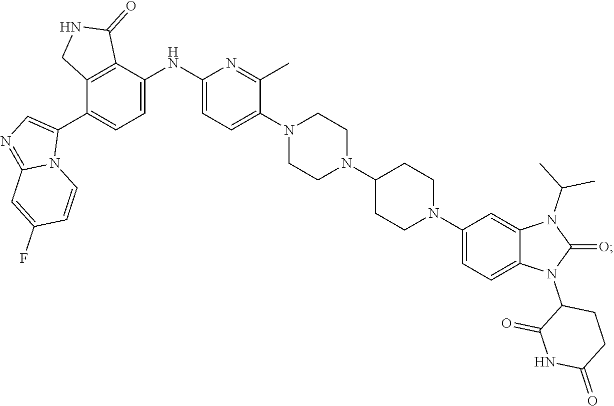 [SMILES: Cc1nc(Nc2ccc(-c3cnc4cc(F)ccn34)c3c2C(=O)NC3)ccc1N1CCN(C2CCN(c3ccc4c(c3)n(C(C)C)c(=O)n4C3CCC(=O)NC3=O)CC2)CC1]
or a pharmaceutically acceptable salt thereof.

15. The compound of claim 3, wherein the compound is: 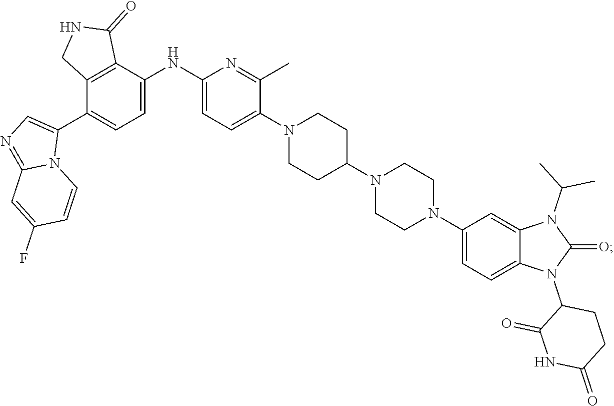 [SMILES: Cc1nc(Nc2ccc(-c3cnc4cc(F)ccn34)c3c2C(=O)NC3)ccc1N1CCC(N2CCN(c3ccc4c(c3)n(C(C)C)c(=O)n4C3CCC(=O)NC3=O)CC2)CC1]
or a pharmaceutically acceptable salt thereof.

16. The compound of claim 3, wherein the compound is: 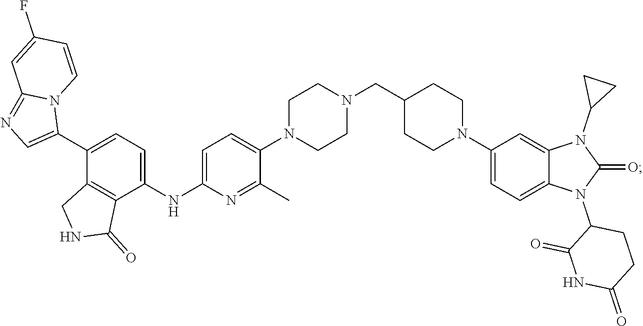 [SMILES: Cc1nc(Nc2ccc(-c3cnc4cc(F)ccn34)c3c2C(=O)NC3)ccc1N1CCN(CC2CCN(c3ccc4c(c3)n(C3CC3)c(=O)n4C3CCC(=O)NC3=O)CC2)CC1]
or a pharmaceutically acceptable salt thereof.

17. The compound of claim 1, wherein the compound is: 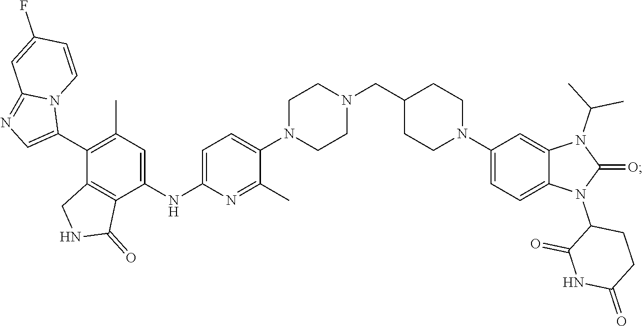 [SMILES: Cc1cc(Nc2ccc(N3CCN(CC4CCN(c5ccc6c(c5)n(C(C)C)c(=O)n6C5CCC(=O)NC5=O)CC4)CC3)c(C)n2)c2c(c1-c1cnc3cc(F)ccn13)CNC2=O]
or a pharmaceutically acceptable salt thereof.

18. A compound, wherein the compound is: 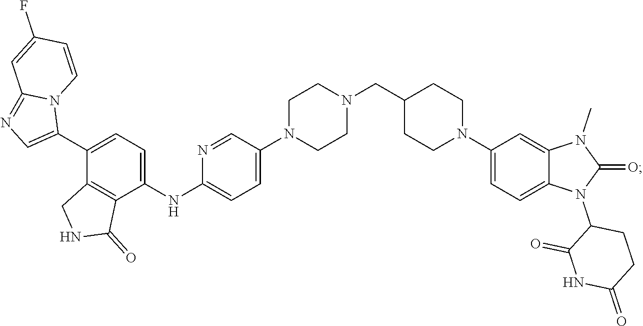 [SMILES: Cn1c(=O)n(C2CCC(=O)NC2=O)c2ccc(N3CCC(CN4CCN(c5ccc(Nc6ccc(-c7cnc8cc(F)ccn78)c7c6C(=O)NC7)nc5)CC4)CC3)cc21]
or a pharmaceutically acceptable salt thereof.

19. A pharmaceutical composition comprising a compound of claim 1, or a pharmaceutically acceptable salt thereof, and a pharmaceutically acceptable carrier.

20. A pharmaceutical composition comprising a compound of claim 18, or a pharmaceutically acceptable salt thereof, and a pharmaceutically acceptable carrier.
